(19) United States
(12) Patent Application Publication
(10) Pub. No.: US 2026/0008787 A1
(43) Pub. Date: Jan. 8, 2026

(54) FUSED RING KRAS INHIBITORS FOR TREATING DISEASE

(71) Applicant: BLOSSOMHILL THERAPEUTICS, INC., San Diego, CA (US)

(72) Inventors: Jingrong Jean CUI, San Diego, CA (US); Eugene Yuanjin RUI, San Diego, CA (US); Evan W. ROGERS, San Diego, CA (US)

(21) Appl. No.: 18/992,359

(22) PCT Filed: Jul. 7, 2023
(86) PCT No.: PCT/US2023/027125

§ 371 Date: Jan. 8, 2025

Related U.S. Application Data
(60) Provisional application No. 63/359,817, filed on Jul. 9, 2022.
(60) Provisional application No. 63/417,684, filed on Oct. 19, 2022.
(60) Provisional application No. 63/502,047, filed on May 12, 2023.
(60) Provisional application No. 63/469,290, filed on May 26, 2023.
(60) Provisional application No. 63/521,487, filed on Jun. 16, 2023.

(51) Int. Cl. C07D 519/00; A61K 31/519; A61K 31/55; A61K 31/553

(52) U.S. Cl. CPC C07D 519/00; A61K 31/519; A61K 31/55; A61K 31/553

(57) ABSTRACT

The present disclosure relates to fused ring compounds targeting KRAS, pharmaceutical compositions containing the compounds, and methods of using such compounds to treat disease, such as cancer.

### RELATED APPLICATIONS

This application claims the benefit of U.S. Provisional Application No. 63/359,817, filed Jul. 9, 2022, U.S. Provisional Application No. 63/417,684, filed Oct. 19, 2022, U.S. Provisional Application No. 63/502,047, filed May 12, 2023, U.S. Provisional Application No. 63/469,290, filed May 26, 2023, and U.S. Provisional Application No. 63/521,487, filed Jun. 16, 2023, the entire disclosures of all of which are incorporated herein by reference.

### TECHNICAL FIELD

The present disclosure relates to fused ring compounds targeting KRAS, pharmaceutical compositions containing the compounds, and methods of using such compounds to treat disease, such as cancer.

### BACKGROUND

Ras is a GTP-binding protein and regulates many important physiologic processes within a cell, such as cell cycle progression, survival, apoptosis, etc. H-Ras, K-Ras, and N-Ras are the main members of Ras superfamily, which are tightly regulated by factors that switch on/off the GTPase activity. Somatic mutations at codons 12, 13 and 61 in the RAS genes are associated with about 16% of all human cancers and KRAS is the most frequently mutated RAS isoform, accounting for 85% of all RAS-related cancers (Prior I. A. et al, A comprehensive survey of Ras mutations in cancer. Cancer Res. 2012, 72, 2457-2467), including 86-96% in pancreatic cancers, 40-50% in colorectal cancers, and 27-39% in lung adenocarcinomas (Kessler D. et al. Drugging an undruggable pocket on KRAS Proc Nat Acad Sci USA. 2019, 116(32):15823-15829). Mutated RAS is locked in the constitutively activated GTP bound state and facilitates enhanced Ras signaling in cancer cells.

Direct targeting of mutant KRAS has previously proven challenging because of its high affinity for nucleotide and the lack of tractable binding pockets for small-molecule inhibitors. Recent successful inhibition of the KRAS G12C mutant by covalent chemical modifiers sotorasib and adagrasib (Stower K, KRAS inhibitors at last, Nature Medicine 2020, 26, 1804) in KRAS G12C mutated lung cancer patients has shed lights on targeting KRAS mutants for therapeutic invention. However, inhibitors targeting KRAS mutants without covalent formation at KRAS G12C are still absent. The increased understanding of structural elements of the KRAS switch II pocket made it possible to design KRAS inhibitors selective for different mutant variants. MRTX1133 has been reported as potent and highly selective noncovalent KRAS G12D inhibitor (Wang X. et al, Identification of MRTX1133, a noncovalent, potent, and selective KRAS^{G12D} inhibitor, J. Med. Chem. 2022, 65: 3123-3133). However, intraperitoneal injection of MRTX1133 was required to achieve sufficient plasma exposure and demonstrate drug efficacy in mice. This suggests MRTX1133 may have poor bioavailability.

Therefore, there is unmet medical need to develop new noncovalent KRAS inhibitors that can selectively target KRAS mutants such as KRAS G12C, KRAS G12D, KRAS G12V, KRAS G12R, KRAS G12S, KRAS G13C, KRAS G13D with good in vivo efficacy, safety, and predicted human oral pharmacokinetic profile for treating patients with KRAS mutant cancers.

### SUMMARY

In one aspect, the disclosure relates to a compound of the formula I, or a pharmaceutically acceptable salt thereof,

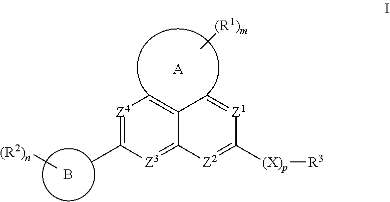

wherein
X is a —O—, —S—, or —NR⁴—;
Z¹ is N or C(R⁵);
Z² is N or C(R⁶)
Z³ is N or C(R⁷);
Z⁴ is N or C(R⁸);
provided that at least two of Z¹-Z⁴ are N;
ring A is a 5- to 8-membered heterocycloalkyl or a C₅-C₈cycloalkyl;
ring B is a 5- to 10-membered heterocycloalkyl, C₆-C₁₀ aryl, or 5- to 10-membered heteroaryl;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{e}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{c}R^{f}, —PR^{c}R^{f}, —P(O)R^{c}R^{f}, —P(O)₂R^{c}R^{f}, —P(O)NR^{c}R^{f}, —P(O)₂NR^{c}R^{f}, —P(O)OR^{c}, —P(O)₂OR^{e}, —CN, or —NO₂;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is —C₁-C₆ alkyl, —C₂-C₆ alkenyl, —C₂-C₆ alkynyl, —C₃-C₆ cycloalkyl, 4- to 10-membered heterocycloalkyl, —C₁-C₆ alkylene-(4- to 10-membered heterocycloalkyl), —C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NRCC(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;

R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NRCS(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each R^{a}, R^{b}, R^{c}, R^{d}, R^{c}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R^{f} taken together with the carbon atom to which they are attached form an oxo groups or an alkenyl;
m is 0, 1, 2, 3, 4, 5, 6, or 7;
n is 0, 1, 2, 3, 4, 5, 6, or 7; and
p is 0 or 1.

In another aspect, the disclosure relates to a compound of the formula 1, or a pharmaceutically acceptable salt thereof,

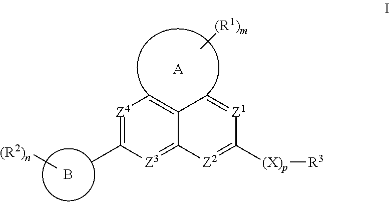

wherein
X is a —O—, —S—, or —NR⁴—;
Z¹ is N or C(R⁵);
Z² is N or C(R⁶);
Z³ is N or C(R⁷);
Z⁴ is N or C(R⁸);
provided that at least two of Z¹-Z⁴ are N;
ring A is a 5- to 8-membered heterocycloalkyl or a C₅-C₅ cycloalkyl;
ring B is a C₆-C₁₀ aryl or 5- to 10-membered heteroaryl;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is —C₁-C₆ alkyl, —C₂-C₆ alkenyl, —C₂-C₆ alkynyl, —C₃-C₆ cycloalkyl, 4- to 10-membered heterocycloalkyl, —C₁-C₆ alkylene-(4- to 10-membered heterocycloalkyl), —C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R¹ and R¹⁰ taken together with the atom or atoms to which they are attached combine to form a monocyclic 4- to 10-membered heterocycloalkyl, a fused bicyclic 5- to 10-membered heterocycloalkyl, or a bridged bicyclic 6- to 10-membered heterocycloalkyl, wherein each hydrogen atom in the monocyclic 4- to 10-membered heterocycloalkyl, fused bicyclic 5- to 10-membered heterocycloalkyl, or a bridged bicyclic 6- to 10-membered heterocycloalkyl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or two hydrogen atoms on a single carbon atom of the monocyclic 4- to 10-membered heterocycloalkyl, fused bicyclic 5- to 10-membered heterocycloalkyl, or bridged bicyclic 6- to 10-membered heterocycloalkyl combine to form an oxo group or an alkenyl group;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)₀C₁-C₆ alkyl, —P(O)₂OC₁-C₆ alkyl, —CN, or —NO₂, or —R^{e} and —R^{f} taken together with the carbon atom to which they are attached form an oxo groups or an alkenyl;
m is 0, 1, 2, 3, 4, 5, 6, or 7;
n is 0, 1, 2, 3, 4, 5, 6, or 7; and
p is 0 or 1.

In some embodiments, at least one hydrogen atom in the compound of the formula I is substituted by a deuterium. In some embodiments, at least one hydrogen atom in R¹, R², or R³ in the compound of the formula I is substituted by a deuterium.

In another aspect, the disclosure provides a compound of the formula II, or a pharmaceutically acceptable salt thereof,

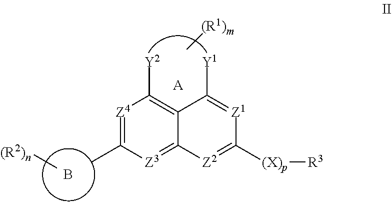

wherein
X is a —O—, —S—, or —NR⁴—;
Y¹ is —O—, —S—, —S(O)—, —S(O)₂—, or —NR^{g}— or —CR¹¹R¹²—;
Y² is —O—, —S—, —S(O)—, —S(O)₂—, —NR¹⁰—, or —CR¹³R¹⁴—;
Z¹ is N or C(R⁵);
Z² is N or C(R⁶)
Z³ is N or C(R⁷);
Z⁴ is N or C(R⁸);
provided that at least two of Z¹-Z⁴ are N;
ring A is a 5- to 8-membered heterocycloalkyl or a C₅-C₈ cycloalkyl;
ring B is a 5- to 10-membered heterocycloalkyl, C₆-C₁₀ aryl or 5- to 10-membered heteroaryl;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{c}, —OS(O)NR^{c}R^{f}, —OS(O)₂NR^{c}R^{f}, —SR^{c}, —S(O)R^{c}, —S(O)₂R^{c}, —S(O)NR^{c}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR'S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is —C₁-C₆ alkyl, —C₂-C₆ alkenyl, —C₂-C₆ alkynyl, —C₃-C₆ cycloalkyl, 4- to 10-membered heterocycloalkyl, —C₁-C₆ alkylene-(4- to 10-membered heterocycloalkyl), —C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{c}S(O)R^{f}, —NR^{c}S(O)₂R^{f}, —NR'S(O)NR^{c}R^{f}, —NR^{c}S(O)₂NR^{c}R^{f}, —C(O)R^{c}, —C(O)OR^{c}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{c}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R¹ and R¹⁰ taken together with the atom or atoms to which they are attached combine to form a monocyclic 4- to 10-membered heterocycloalkyl, a fused bicyclic 5- to 10-membered heterocycloalkyl, or a bridged bicyclic 6- to 10-membered heterocycloalkyl, wherein each hydrogen atom in the monocyclic 4- to 10-membered heterocycloalkyl, fused bicyclic 5- to 10-membered heterocycloalkyl, or a bridged bicyclic 6- to 10-membered heterocycloalkyl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or two hydrogen atoms on a single carbon atom of the monocyclic 4- to 10-membered heterocycloalkyl, fused bicyclic 5- to 10-membered heterocycloalkyl, or bridged bicyclic 6- to 10-membered heterocycloalkyl combine to form an oxo group or an alkenyl group;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R⁸, —S(O)₂R⁸, —S(O)NR^{g}R^{h}, —S(O)₂NR^{g}R^{h}, —OS(O)NR^{g}R^{h}, —OS(O)₂NR⁸R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each R⁹ and R¹⁰ is independently H, deuterium, —C(O)R^{g}, —C(O)NR^{g}R^{h}, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R^{g} and a R^{f} or R¹⁰ and a R^{f}, taken together with the atoms to which they are attached, combine to form a 4- to 10-membered heterocycloalkyl, wherein each hydrogen atom in the 4- to 10-membered heterocycloalkyl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or two hydrogen atoms on a single carbon atom of the 4- to 10-membered heterocycloalkyl combine to form an oxo group or an alkenyl group;
each of R¹¹, R¹², R¹³, and R¹⁴ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or a R¹ and R¹¹, a R¹ and R¹², a R¹ and R¹³, a R¹ and R¹⁴, two of R¹¹ and R¹², or two of R¹³ and R¹⁴ taken together with the carbon or carbons to which they are attached, combine to form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, or two of R¹¹ and R¹² or R¹³ and R¹⁴ taken together with the carbon atom to which they are attached form an oxo group or an C₂-C₆ alkenyl group;
each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{c} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R^{f} taken together with the carbon atom to which they are attached form an oxo groups or an alkenyl;
m is 0, 1, 2, 3, 4, 5, 6, or 7;
n is 0, 1, 2, 3, 4, 5, 6, or 7; and
p is 0 or 1.

In another aspect, the disclosure provides a compound of the formula II, or a pharmaceutically acceptable salt thereof,

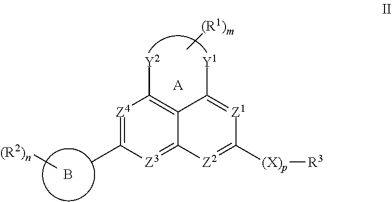

wherein
X is a —O—, —S—, or —NR⁴—;
Y¹ is —O—, —S—, —S(O)—, —S(O)₂—, —NR^{g}— or —CR¹¹R¹²—;
Y² is —O—, —S—, —S(O)—, —S(O)₂—, —NR¹⁰—, or —CR¹³R¹⁴—;
Z¹ is N or C(R⁵);
Z² is N or C(R⁶)
Z³ is N or C(R⁷);
Z⁴ is N or C(R⁸)
provided that at least two of Z¹-Z⁴ are N;
ring A is a 5- to 8-membered heterocycloalkyl or a C₅-C₈ cycloalkyl;
ring B is a C₆-C₁₀ aryl or 5- to 10-membered heteroaryl;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR'S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R¹, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is —C₁-C₆ alkyl, —C₂-C₆ alkenyl, —C₂-C₆ alkynyl, —C₃-C₆ cycloalkyl, 4- to 10-membered heterocycloalkyl, —C₁-C₆ alkylene-(4- to 10-membered heterocycloalkyl), —C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆alkyl, C₁-C₆haloalkyl, —OR^{c}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R¹ and R¹⁰ taken together with the atom or atoms to which they are attached combine to form a monocyclic 4- to 10-membered heterocycloalkyl, a fused bicyclic 5- to 10-membered heterocycloalkyl, or a bridged bicyclic 6- to 10-membered heterocycloalkyl, wherein each hydrogen atom in the monocyclic 4- to 10-membered heterocycloalkyl, fused bicyclic 5- to 10-membered heterocycloalkyl, or a bridged bicyclic 6- to 10-membered heterocycloalkyl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₇-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or two hydrogen atoms on a single carbon atom of the monocyclic 4- to 10-membered heterocycloalkyl, fused bicyclic 5- to 10-membered heterocycloalkyl, or bridged bicyclic 6- to 10-membered heterocycloalkyl combine to form an oxo group or an alkenyl group;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{b}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each R⁹ and R¹⁰ is independently H, deuterium, —C(O)R^{g}, —C(O)NR^{g}R^{h}, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R^{g} and a R¹ or R¹⁰ and a R¹, taken together with the atoms to which they are attached, combine to form a 4- to 10-membered heterocycloalkyl, wherein each hydrogen atom in the 4- to 10-membered heterocycloalkyl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or two hydrogen atoms on a single carbon atom of the 4- to 10-membered heterocycloalkyl combine to form an oxo group or an alkenyl group;
each of R¹¹, R¹², R¹³, and R¹⁴ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or a R¹ and R¹¹, a R¹ and R¹², a R¹ and R¹³, a R¹ and R¹⁴, two of R¹¹ and R¹², or two of R¹³ and R¹⁴ taken together with the carbon or carbons to which they are attached, combine to form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, or two of R¹¹ and R¹² or R¹³ and R¹⁴ taken together with the carbon atom to which they are attached form an oxo group or an C₂-C₆ alkenyl group;
each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆alkyl, C₁-C₆haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R^{f} taken together with the carbon atom to which they are attached form an oxo groups or an alkenyl;
m is 0, 1, 2, 3, 4, 5, 6, or 7;
n is 0, 1, 2, 3, 4, 5, 6, or 7; and
p is 0 or 1.

In some embodiments, at least one hydrogen atom in the compound of the formula II is substituted by a deuterium. In some embodiments, at least one hydrogen atom in R¹, R², or R³ in the compound of the formula II is substituted by a deuterium.

In some embodiments, the disclosure provides a compound of the formula III, or a pharmaceutically acceptable salt thereof,

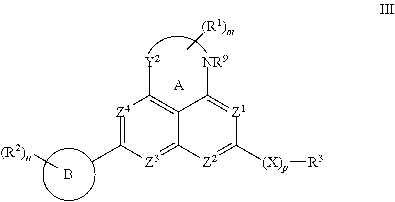

wherein R¹, R², R³, R⁹, A, B, X, Y², Z¹, Z², Z³, Z⁴, m, n, and p are as described herein.

In some embodiments, the disclosure provides a compound of the formula IV, or a pharmaceutically acceptable salt thereof,

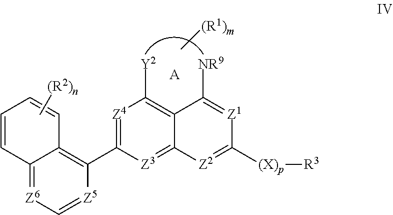

wherein R¹, R², R³, R⁹, A, X, Y², Z¹, Z², Z³, Z⁴, Z⁵, Z⁶, m, n, and p are as described herein.

In some embodiments, the disclosure provides a compound of the formula IV, or a pharmaceutically acceptable salt thereof,

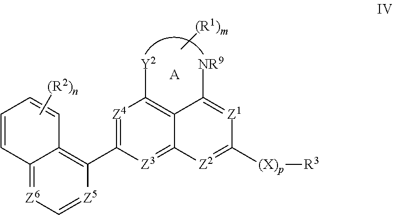

wherein R¹, R², R³, R⁹, A, X, Y², Z¹, Z², Z³, Z⁴, m, n, and p are as described herein, and Z⁵ is N or C(R¹⁵), and Z⁶ is N or C(R⁶).

In some embodiments, the disclosure provides a compound of the formula V, or a pharmaceutically acceptable salt thereof,

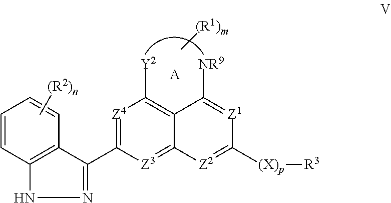

wherein R¹, R², R³, R⁹, A, X, Y², Z¹, Z², Z³, Z⁴, m, n, and p are as described herein.

In some embodiments, the disclosure provides a compound of the formula VI, or a pharmaceutically acceptable salt thereof,

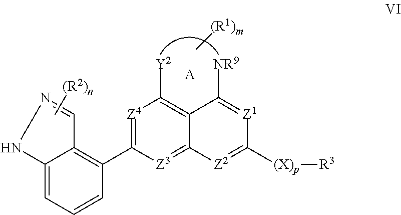

wherein R¹, R², R³, R⁹, A, X, Y², Z¹, Z², Z³, Z⁴, m, n, and p are as described herein.

In some embodiments, the disclosure provides a compound of the formula VII, or a pharmaceutically acceptable salt thereof,

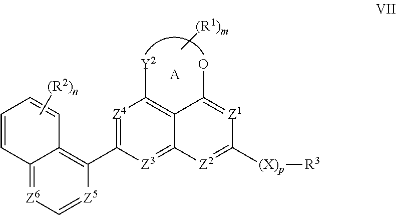

wherein R¹, R², R³, A, X, Y², Z¹, Z², Z³, Z⁴, Z⁵, Z⁶, m, n, and p are as described herein.

In some embodiments, the disclosure provides a compound of the formula VII, or a pharmaceutically acceptable salt thereof,

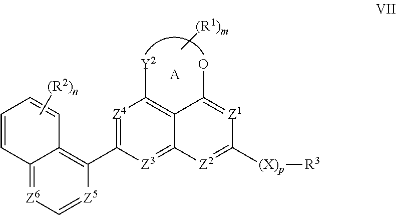

wherein R¹, R², R³, A, X, Y², Z¹, Z², Z³, Z⁴, m, n, and p are as described herein, and Z⁵ is N or C(R¹⁵), and Z⁶ is N or C(R¹⁶).

In some embodiments, the disclosure provides a compound of the formula VIII, or a pharmaceutically acceptable salt thereof,

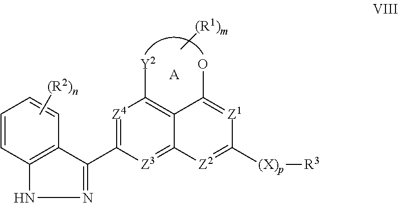

wherein R¹, R², R³, A, X, Y², Z¹, Z², Z³, Z⁴, m, n, and p are as described herein.

In some embodiments, the disclosure provides a compound of the formula IX, or a pharmaceutically acceptable salt thereof,

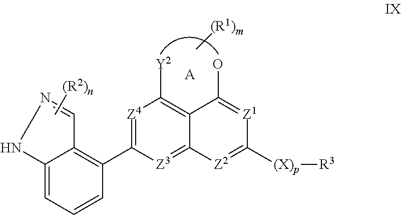

wherein R¹, R², R³, A, X, Y², Z¹, Z², Z³, Z⁴, m, n, and p are as described herein.

In some embodiments, the disclosure provides a compound of the formula X, or a pharmaceutically acceptable salt thereof,

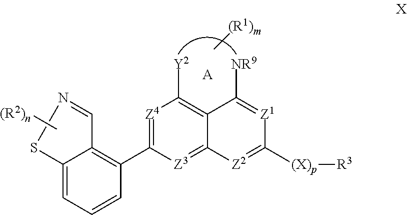

wherein R¹, R², R³, R⁹, A, X, Y², Z¹, Z², Z³, Z⁴, m, n, and p are as described herein.

In some embodiments, the disclosure provides a compound of the formula XI, or a pharmaceutically acceptable salt thereof,

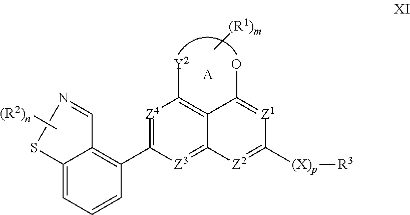

wherein R¹, R², R³, A, X, Y², Z¹, Z², Z³, Z⁴, m, n, and p are as described herein.

In some embodiments, the disclosure provides a compound of the formula XII, or a pharmaceutically acceptable salt thereof,

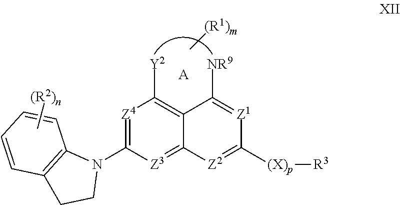

wherein R¹, R², R³, R⁹, A, X, Y², Z¹, Z², Z³, Z⁴, m, n, and p are as described herein.

In some embodiments, the disclosure provides a compound of the formula XIII, or a pharmaceutically acceptable salt thereof,

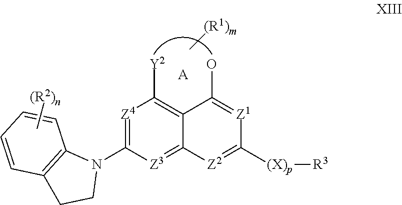

wherein R¹, R², R³, A, X, Y², Z¹, Z², Z³, Z⁴, m, n, and p are as described herein.

In some embodiments, the disclosure provides a compound of the formula XIV, or a pharmaceutically acceptable salt thereof,

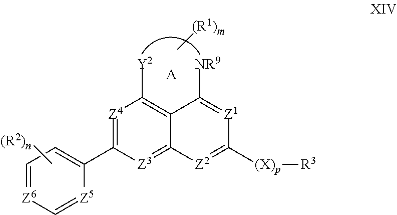

wherein R¹, R², R³, R⁹, A, X, Y², Z¹, Z², Z³, Z⁴, Z⁵, Z⁶, m, n, and p are as described herein.

In some embodiments, the disclosure provides a compound of the formula XIV, or a pharmaceutically acceptable salt thereof,

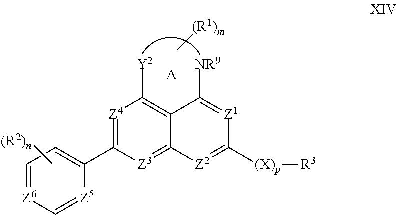

wherein R¹, R², R³, R⁹, A, X, Y², Z¹, Z², Z³, Z⁴, m, n, and p are as described herein; Z⁵ is N or C(R¹⁵); and Z⁶ is N or C(R¹⁶).

In some embodiments, the disclosure provides a compound of the formula XV, or a pharmaceutically acceptable salt thereof,

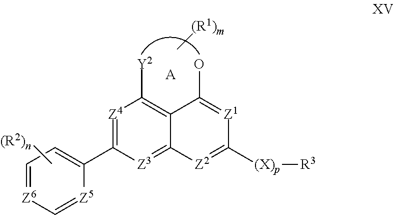

wherein R¹, R², R³, R⁹, A, X, Y², Z¹, Z², Z³, Z⁴, Z⁵, Z⁶, m, n, and p are as described herein.

In some embodiments, the disclosure provides a compound of the formula XV, or a pharmaceutically acceptable salt thereof,

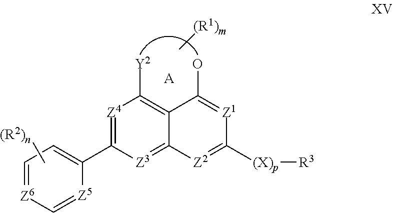

wherein R¹, R², R³, R⁹, A, X, Y², Z¹, Z², Z³, Z⁴, m, n, and p are as described herein; Z⁵ is N or C(R¹⁵); and Z⁶ is N or C(R¹⁶).

In some embodiments, the disclosure provides a compound of the formula XVI, or a pharmaceutically acceptable salt thereof,

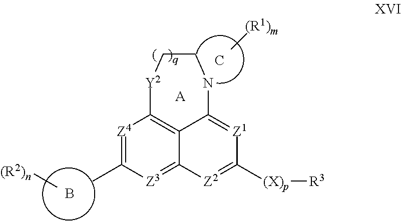

wherein R¹, R², R³, A, B, X, Y², Z¹, Z², Z³, Z⁴, Z⁵, Z⁶, m, n, and p are as described herein, and ring C is a 4- to 8-membered heterocycloalkyl, and q is 0, 1, or 2.

In some embodiments, the disclosure provides a compound of the formula XVII, or a pharmaceutically acceptable salt thereof,

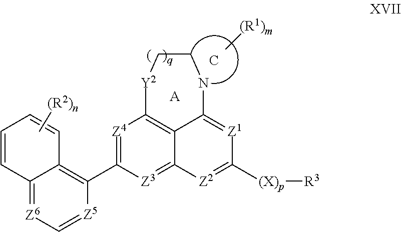

wherein R¹, R², R³, A, X, Y², Z¹, Z², Z³, Z⁴, Z⁵, Z⁶, m, n, and p are as described herein, and ring C is a 4- to 8-membered heterocycloalkyl, and q is 0, 1, or 2.

In some embodiments, the disclosure provides a compound of the formula XVII, or a pharmaceutically acceptable salt thereof,

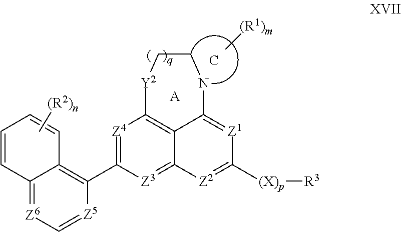

wherein R¹, R², R³, A, X, Y², Z¹, Z², Z³, Z⁴, m, n, and p are as described herein; ring C is a 4- to 8-membered heterocycloalkyl; q is 0, 1, or 2; Z⁵ is N or C(R¹⁵); and Z⁶ is N or C(R¹⁶)

In some embodiments, the disclosure provides a compound of the formula XVIII, or a pharmaceutically acceptable salt thereof,

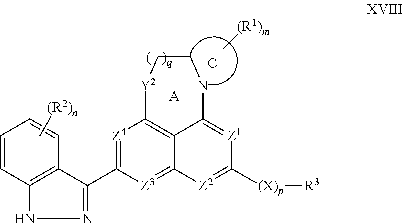

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XIX, or a pharmaceutically acceptable salt thereof,

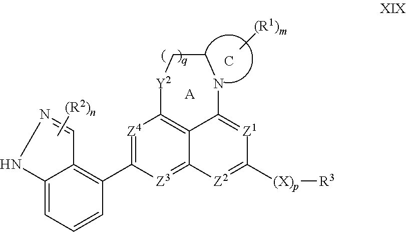

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XX, or a pharmaceutically acceptable salt thereof,

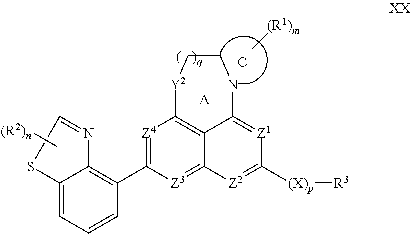

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XXI, or a pharmaceutically acceptable salt thereof,

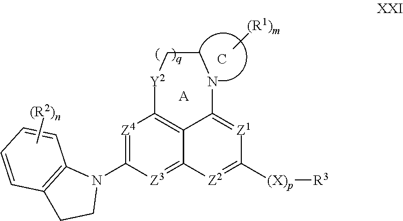

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XXII, or a pharmaceutically acceptable salt thereof,

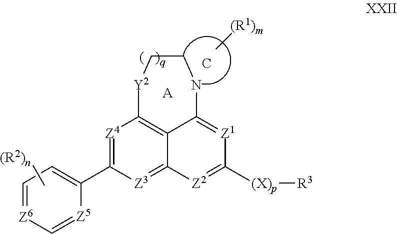

wherein R¹, R², R³, A, X, Y², Z¹, Z², Z³, Z⁴, Z⁵, Z⁶, m, n, and p are as described herein, and ring C is a 4- to 8-membered heterocycloalkyl, and q is 0, 1, or 2.

In some embodiments, the disclosure provides a compound of the formula XXII, or a pharmaceutically acceptable salt thereof,

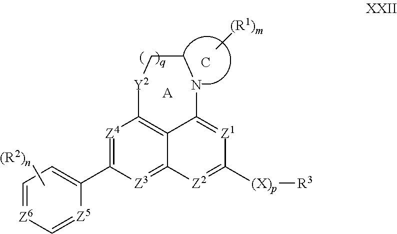

wherein R¹, R², R³, A, X, Y², Z¹, Z², Z³, Z⁴, m, n, and p are as described herein; ring C is a 4- to 8-membered heterocycloalkyl; q is 0, 1, or 2; Z⁵ is N or C(R¹⁵); and Z⁶ is N or C(R¹⁶)

In some embodiments, the disclosure provides a compound of the formula XXIII, or a pharmaceutically acceptable salt thereof,

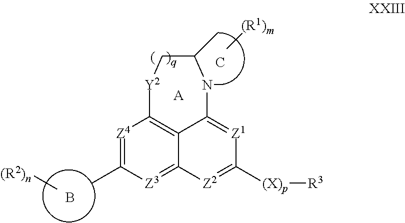

wherein R¹, R², R³, A, B, X, Y², Z¹, Z², Z³, Z⁴, Z⁵, Z⁶, m, n, and p are as described herein, and ring C is a 4- to 8-membered heterocycloalkyl, and q is 0, 1, or 2.

In some embodiments, the disclosure provides a compound of the formula XXIV, or a pharmaceutically acceptable salt thereof,

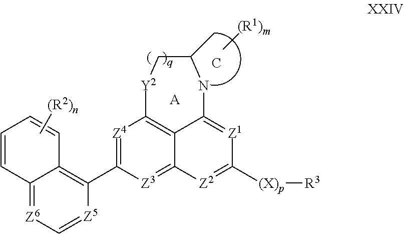

wherein R¹, R², R³, A, X, Y², Z¹, Z², Z³, Z⁴, Z⁵, Z⁶, m, n, and p are as described herein, and ring C is a 4- to 8-membered heterocycloalkyl, and q is 0, 1, or 2.

In some embodiments, the disclosure provides a compound of the formula XXIV, or a pharmaceutically acceptable salt thereof,

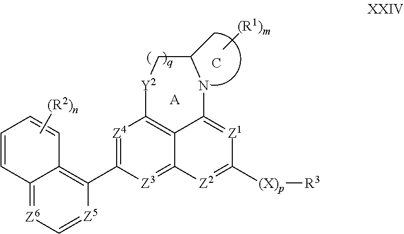

wherein R¹, R², R³, A, X, Y², Z¹, Z², Z³, Z⁴, m, n, and p are as described herein; ring C is a 4- to 8-membered heterocycloalkyl; q is 0, 1, or 2; Z⁵ is N or C(R¹⁵); and Z⁶ is N or C(R¹⁶)

In some embodiments, the disclosure provides a compound of the formula XXV, or a pharmaceutically acceptable salt thereof,

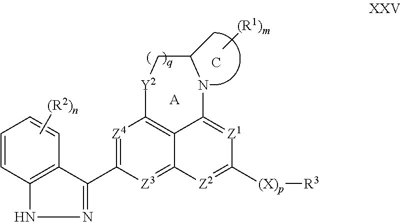

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XXVI, or a pharmaceutically acceptable salt thereof,

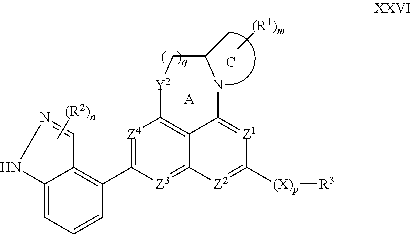

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XXVII, or a pharmaceutically acceptable salt thereof,

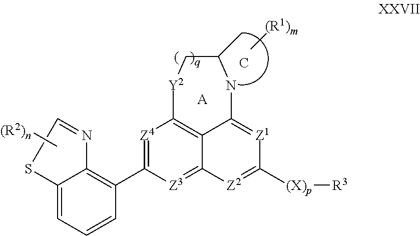

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XXVIII, or a pharmaceutically acceptable salt thereof,

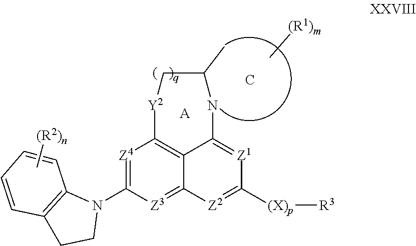

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XXIX, or a pharmaceutically acceptable salt thereof,

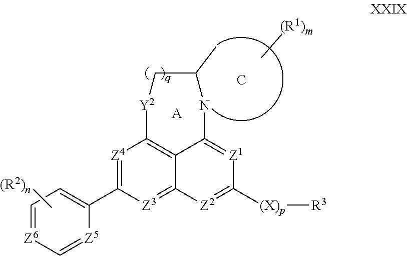

wherein R¹, R², R³, A, X, Y², Z¹, Z², Z³, Z⁴, Z⁵, Z⁶, m, n, and p are as described herein, and ring C is a 4- to 8-membered heterocycloalkyl, and q is 0, 1, or 2.

In some embodiments, the disclosure provides a compound of the formula XXIX, or a pharmaceutically acceptable salt thereof,

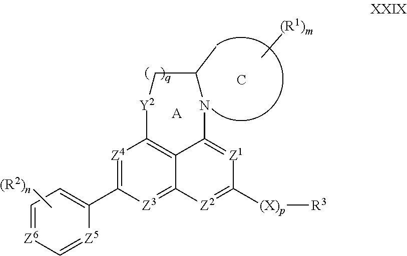

wherein R¹, R², R³, A, X, Y², Z¹, Z², Z³, Z⁴, m, n, and p are as described herein; ring C is a 4- to 8-membered heterocycloalkyl; q is 0, 1, or 2; Z⁵ is N or C(R¹⁵); and Z⁶ is N or C(R¹⁶).

In some embodiments, the disclosure provides a compound of the formula XXX, or a pharmaceutically acceptable salt thereof,

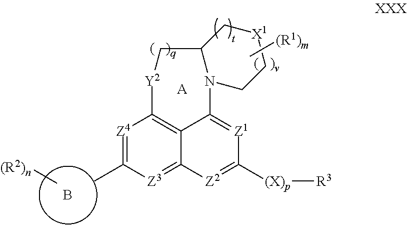

wherein R¹, R², R³, A, B, X, Y², Z¹, Z², Z³, Z⁴, Z⁵, Z⁶, m, n, and p are as described herein, and X¹ is —O—, —NH—, or —CH₂—, q is 0, 1, or 2, t is 1 or 2, and v is 1 or 2.

In some embodiments, the disclosure provides a compound of the formula XXXI, or a pharmaceutically acceptable salt thereof,

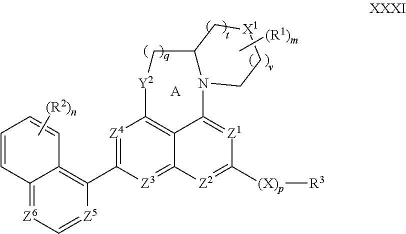

wherein R¹, R², R³, A, X, Y², Z¹, Z², Z³, Z⁴, Z⁵, Z⁶, m, n, and p are as described herein, and X¹ is —O— or —CH₂—, q is 0, 1, or 2, t is 1 or 2, and v is 1 or 2.

In some embodiments, the disclosure provides a compound of the formula XXXI, or a pharmaceutically acceptable salt thereof,

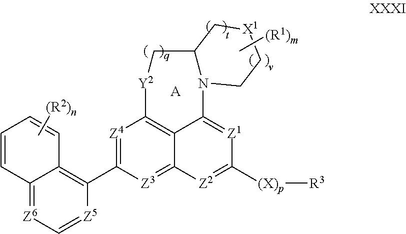

wherein R¹, R², R³, A, X, Y², Z¹, Z², Z³, Z⁴, m, n, and p are as described herein, and X¹ is —O— or —CH₂—, q is 0, 1, or 2, t is 1 or 2, v is 1 or 2, Z⁵ is N or C(R¹⁵), and Z⁶ is N or C(R¹⁶)

In some embodiments, the disclosure provides a compound of the formula XXXII, or a pharmaceutically acceptable salt thereof,

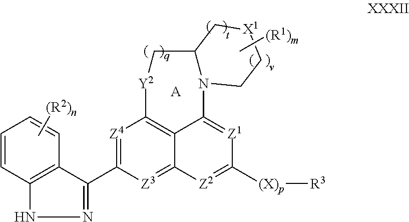

wherein R¹, R², R³, A, X, Y², Z¹, Z², Z³, Z⁴, m, n, and p are as described herein, and X¹ is —O— or —CH₂—, q is 0, 1, or 2, t is 1 or 2, and v is 1 or 2.

In some embodiments, the disclosure provides a compound of the formula XXXIII, or a pharmaceutically acceptable salt thereof,

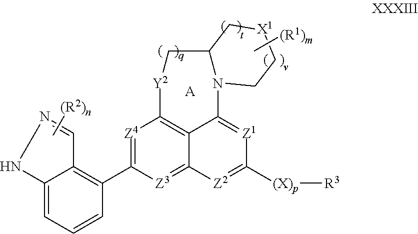

wherein R¹, R², R³, A, X, Y², Z¹, Z², Z³, Z⁴, m, n, and p are as described herein, and X¹ is —O—, —NH—, or —CH₂—, q is 0, 1, or 2, t is 1 or 2, and v is 1 or 2.

In some embodiments, the disclosure provides a compound of the formula XXXIV, or a pharmaceutically acceptable salt thereof,

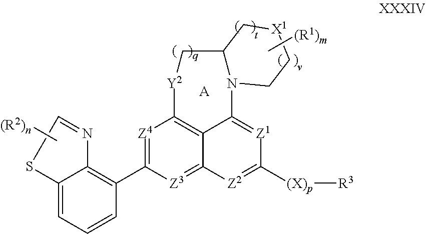

wherein R¹, R², R³, A, X, Y², Z¹, Z², Z³, Z⁴, m, n, and p are as described herein, and X¹ is —O— or —CH₂—, q is 0, 1, or 2, t is 1 or 2, and v is 1 or 2.

In some embodiments, the disclosure provides a compound of the formula XXXV, or a pharmaceutically acceptable salt thereof,

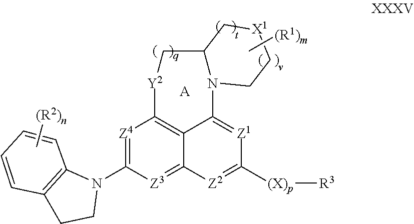

wherein R¹, R², R³, A, X, Y², Z¹, Z², Z³, Z⁴, m, n, and p are as described herein, and X¹ is —O— or —CH₂—, q is 0, 1, or 2, t is 1 or 2, and v is 1 or 2.

In some embodiments, the disclosure provides a compound of the formula XXXVI, or a pharmaceutically acceptable salt thereof,

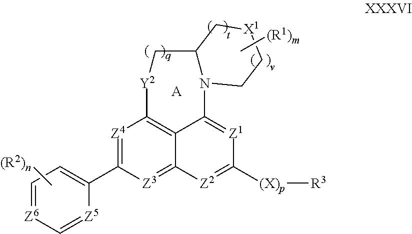

wherein R¹, R², R³, A, X, Y², Z¹, Z², Z³, Z⁴, Z⁵, Z⁶, m, n, and p are as described herein, and X¹ is —O— or —CH₂—, q is 0, 1, or 2, t is 1 or 2, and v is 1 or 2.

In some embodiments, the disclosure provides a compound of the formula XXXVI, or a pharmaceutically acceptable salt thereof,

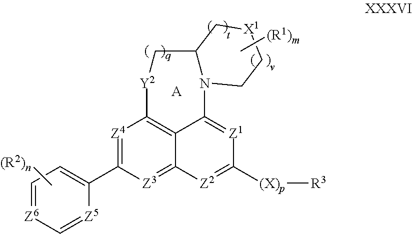

wherein R¹, R², R³, A, X, Y², Z¹, Z², Z³, Z⁴, m, n, and p are as described herein, and X¹ is —O— or —CH₂—, q is 0, 1, or 2, t is 1 or 2, v is 1 or 2, Z⁵ is N or C(R¹⁵), and Z⁶ is N or C(R¹⁶)

In some embodiments, the disclosure provides a compound of the formula XXXVII, or a pharmaceutically acceptable salt thereof,

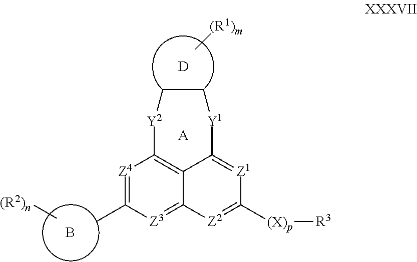

wherein R¹, R², R³, A, B, X, Y², Y², Z¹, Z², Z³, Z⁴, Z⁵, Z⁶, m, n, and p are as described herein, and ring D is a C₃-C₆ cycloalkyl or a 4- to 8-membered heterocycloalkyl.

In some embodiments, the disclosure provides a compound of the formula XXXVIII, or a pharmaceutically acceptable salt thereof,

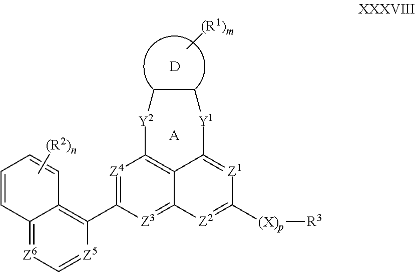

wherein R¹, R², R³, A, X, Y¹, Y², Z¹, Z², Z³, Z⁴, Z⁵, Z⁶, m, n, and p are as described herein, and ring D is a C₃-C₆ cycloalkyl or a 4- to 8-membered heterocycloalkyl.

In some embodiments, the disclosure provides a compound of the formula XXXVIII, or a pharmaceutically acceptable salt thereof,

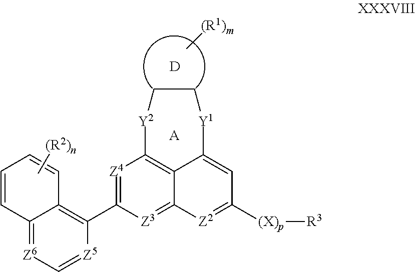

wherein R¹, R², R³, A, X, Y¹, Y², Z¹, Z², Z³, Z⁴, m, n, and p are as described herein, and ring D is a C₃-C₆ cycloalkyl or a 4- to 8-membered heterocycloalkyl, Z⁵ is N or C(R¹⁵), and Z⁶ is N or C(R¹⁶)

In some embodiments, the disclosure provides a compound of the formula XXXIX, or a pharmaceutically acceptable salt thereof,

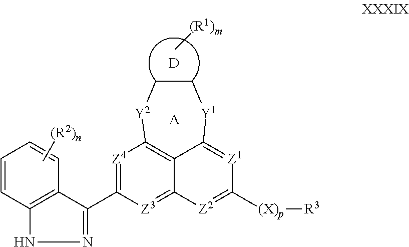

wherein R¹, R², R³, A, X, Y¹, Y², Z¹, Z², Z³, Z⁴, m, n, and p are as described herein, and ring D is a C₃-C₆ cycloalkyl or a 4- to 8-membered heterocycloalkyl.

In some embodiments, the disclosure provides a compound of the formula XXXX, or a pharmaceutically acceptable salt thereof,

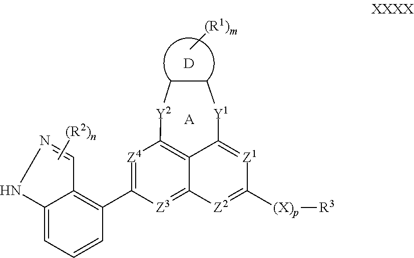

wherein R¹, R², R³, A, X, Y¹, Y², Z¹, Z², Z³, Z⁴, m, n, and p are as described herein, and ring D is a C₃-C₆ cycloalkyl or a 4- to 8-membered heterocycloalkyl.

In some embodiments, the disclosure provides a compound of the formula XXXXI, or a pharmaceutically acceptable salt thereof,

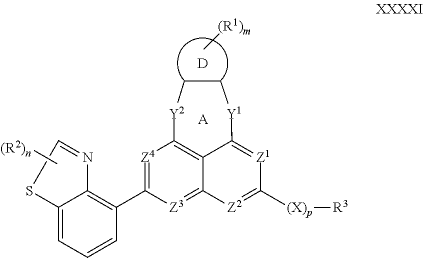

wherein R¹, R², R³, A, X, Y¹, Y², Z¹, Z², Z³, Z⁴, m, n, and p are as described herein, and ring D is a C₃-C₆ cycloalkyl or a 4- to 8-membered heterocycloalkyl.

In some embodiments, the disclosure provides a compound of the formula XXXXII, or a pharmaceutically acceptable salt thereof,

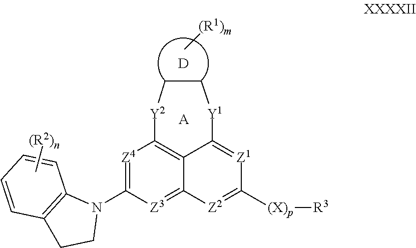

wherein R¹, R², R³, A, X, Y¹, Y², Z¹, Z², Z³, Z⁴, m, n, and p are as described herein, and ring D is a C₃-C₆ cycloalkyl or a 4- to 8-membered heterocycloalkyl.

In some embodiments, the disclosure provides a compound of the formula XXXXIII, or a pharmaceutically acceptable salt thereof,

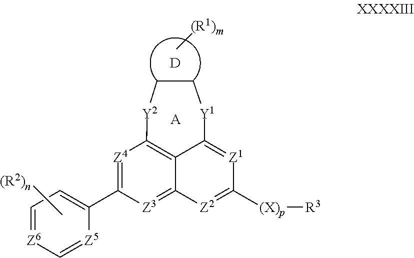

wherein R¹, R², R³, R⁹, A, X, Y¹, Y², Z¹, Z², Z³, Z⁴, Z⁵, Z⁶, m, n, and p are as described herein, and ring D is a C₃-C₆ cycloalkyl or a 4- to 8-membered heterocycloalkyl.

In some embodiments, the disclosure provides a compound of the formula XXXXIII, or a pharmaceutically acceptable salt thereof,

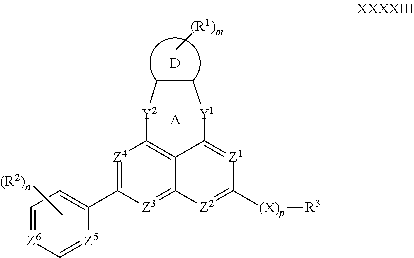

wherein R¹, R², R³, R⁹, A, X, Y¹, Y², Z¹, Z², Z³, Z⁴, m, n, and p are as described herein, and ring D is a C₃-C₆ cycloalkyl or a 4- to 8-membered heterocycloalkyl, Z⁵ is N or C(R¹⁵), and Z⁶ is N or C(R¹⁶)

In some embodiments, the disclosure provides a compound of the formula XXXXIV, or a pharmaceutically acceptable salt thereof,

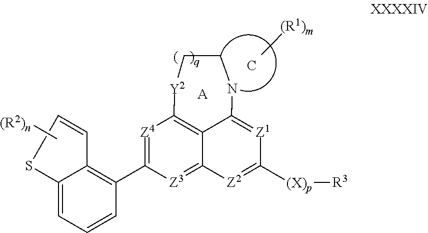

wherein R¹, R², R³, A, X, Y², Z¹, Z², Z³, Z⁴, m, n, and p are as described herein, and wherein ring C is a 4- to 8-membered heterocycloalkyl; and q is 0, 1, or 2.

In some embodiments, the disclosure provides a compound of the formula XXXXV, or a pharmaceutically acceptable salt thereof,

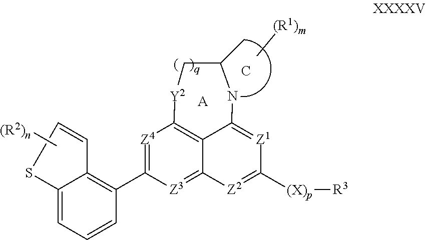

wherein R¹, R², R³, A, X, Y², Z¹, Z², Z³, Z⁴, m, n, and p are as described herein, and wherein ring C is a 4- to 8-membered heterocycloalkyl; and q is 0, 1, or 2.

In some embodiments, the disclosure provides a compound of the formula XXXXVI, or a pharmaceutically acceptable salt thereof,

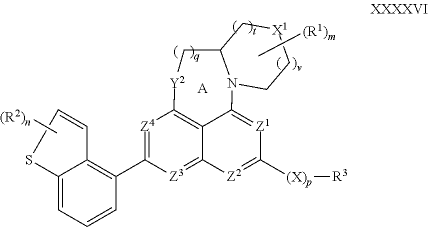

wherein R¹, R², R³, A, X, Y², Z¹, Z², Z³, Z⁴, m, n, and p are as described herein, and wherein ring C is a 4- to 8-membered heterocycloalkyl; X¹ is —O—, —NH— or —CH₂—; q is 0, 1, or 2; t is 1 or 2, and v is 1 or 2.

In certain embodiments of the above aspects, the compound of Formula (I)—(XXXXVI) is a compound selected from those species described or exemplified in the detailed description below.

In further aspects, the disclosure relates to a pharmaceutical composition comprising at least one compound of Formula (I)—(XXXXVI) or a pharmaceutically acceptable salt thereof. Pharmaceutical compositions according to the disclosure may further comprise a pharmaceutically acceptable excipient.

In further aspects, the disclosure relates to a compound of Formula (I)—(XXXXVI), or a pharmaceutically acceptable salt thereof, for use as a medicament.

In further aspects, the disclosure relates to a method of treating disease, such as cancer comprising administering to a subject in need of such treatment an effective amount of at least one compound of Formula (I)—(XXXXVI), or a pharmaceutically acceptable salt thereof.

In further aspects, the disclosure relates to use of a compound of Formula (I)—(XXXXVI), or a pharmaceutically acceptable salt thereof, in the preparation of a medicament for the treatment of disease, such as cancer, and the use of such compounds and salts for treatment of such diseases.

In further aspects, the disclosure relates to a method of inhibiting a Ras, such as K-Ras, comprising contacting a cell comprising one or more of Ras with an effective amount of at least one compound of Formula (I)—(XXXXVI), or a pharmaceutically acceptable salt thereof, and/or with at least one pharmaceutical composition of the disclosure, wherein the contacting is in vitro, ex vivo, or in vivo.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A compound of the formula I, or a pharmaceutically acceptable salt thereof,

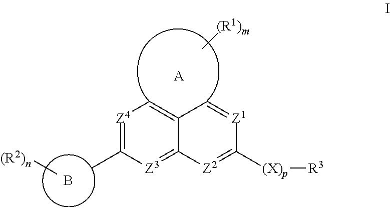

wherein
X is a —O—, —S—, or —NR⁴—;
Z¹ is N or C(R⁵);
Z² is N or C(R⁶)
Z³ is N or C(R⁷);
Z⁴ is N or C(R⁸);
provided that at least two of Z¹-Z⁴ are N;
ring A is a 5- to 8-membered heterocycloalkyl or a C₅-C₈ cycloalkyl;
ring B is a C₆-C₁₀ aryl or 5- to 10-membered heteroaryl;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{e}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{e}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is —C₁-C₆ alkyl, —C₂-C₆ alkenyl, —C₂-C₆ alkynyl, —C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆alkyl, —C₁-C₆alkylene-O—C₁-C₆alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{c}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁—C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{e} and —R^{f} taken together with the carbon atom to which they are attached form an oxo groups or an alkenyl;
m is 0, 1, 2, 3, 4, 5, 6, or 7;
n is 0, 1, 2, 3, 4, 5, 6, or 7; and
p is 0 or 1.

2. The compound of clause 1, or a pharmaceutically acceptable salt thereof, wherein the compound is of the formula II,

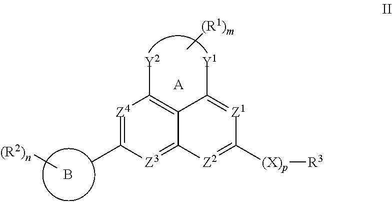

wherein
Y¹ is —O—, —S—, —S(O)—, —S(O)₂—, —NR⁹— or —CR¹¹R¹²—;
Y² is —O—, —S—, —S(O)—, —S(O)₂—, —NR¹⁰—, or —CR¹³R¹⁴—;
each R⁹ and R¹⁰ is independently H, deuterium, —C(O)R^{g}, —C(O)NR^{g}R^{h}, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e′}, —CN, or —NO₂; or R⁹ and a R¹ or R¹⁰ and a R¹, taken together with the atoms to which they are attached, combine to form a 4- to 10-membered heterocycloalkyl, wherein each hydrogen atom in the 4- to 10-membered heterocycloalkyl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or two hydrogen atoms on a single carbon atom of the 4- to 10-membered heterocycloalkyl combine to form an oxo group or an alkenyl group; and
each of R¹¹, R¹², R¹³, and R¹⁴ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or a R¹ and R¹¹, a R¹ and R¹², a R¹ and R¹³, a R¹ and R¹⁴, two of R¹¹ and R¹², or two of R¹³ and R¹⁴ taken together with the carbon or carbons to which they are attached, combine to form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}(O)OR^{f}, —NR^{e}C(O)NR^{e}R^{f}, —NR^{e}S(O)R^{f}. —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, or two of R¹¹ and R¹² or R¹³ and R¹⁴ taken together with the carbon atom to which they are attached form an oxo group or an C₂-C₆ alkenyl group.

3. The compound of clause 1, or a pharmaceutically acceptable salt thereof, wherein the compound is of the formula XXXVII, XXXVIII, XXXIX, XXXX, XXXXI, XXXXII, or XXXXIII

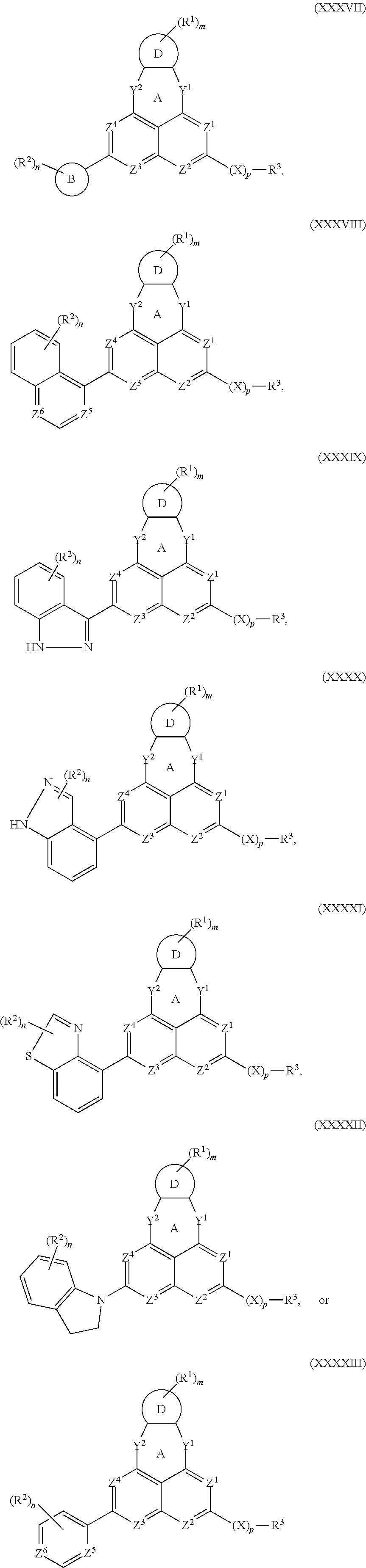

wherein
Y¹ is —O—, —S—, —S(O)—, —S(O)₂—, —NR⁹— or —CR¹¹R¹²—;
Y² is —O—, —S—, —S(O)—, —S(O)₂—, —NR¹⁰—, or —CR¹³R¹⁴—;
Z⁵ is N or C(R¹⁵);
Z⁶ is N or C(R¹⁶)
ring D is a C₃-C₆ cycloalkyl or a 4- to 8-membered heterocycloalkyl;
each R⁹ and R¹⁰ is independently H, deuterium, —C(O)R^{g}, —C(O)NR^{g}R^{h}, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{c}, —CN, or —NO₂; or R⁹ and a R¹ or R¹⁰ and a R¹, taken together with the atoms to which they are attached, combine to form a 4- to 10-membered heterocycloalkyl, wherein each hydrogen atom in the 4- to 10-membered heterocycloalkyl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or two hydrogen atoms on a single carbon atom of the 4- to 10-membered heterocycloalkyl combine to form an oxo group or a C₂-C₆ alkenyl group;
each of R¹¹, R¹², R¹³, and R¹⁴ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or a R¹ and R¹¹, a R¹ and R¹², a R¹ and R¹³, a R¹ and R¹⁴, two of R¹¹ and R¹², or two of R³ and R¹⁴ taken together with the carbon or carbons to which they are attached, combine to form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, or two of R¹¹ and R¹² or R¹³ and R¹⁴ taken together with the carbon atom to which they are attached form an oxo group or an C₂-C₆ alkenyl group; and
each of R¹⁵ and R¹⁶ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂.

4. The compound of clause 1 or 2, wherein the compound is of the formula III

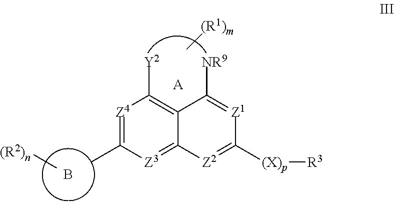

or a pharmaceutically acceptable salt thereof.

5. The compound of clause 1 or 2, or a pharmaceutically acceptable salt thereof, wherein the compound is of the formula XVI, XXIII, or XXX,

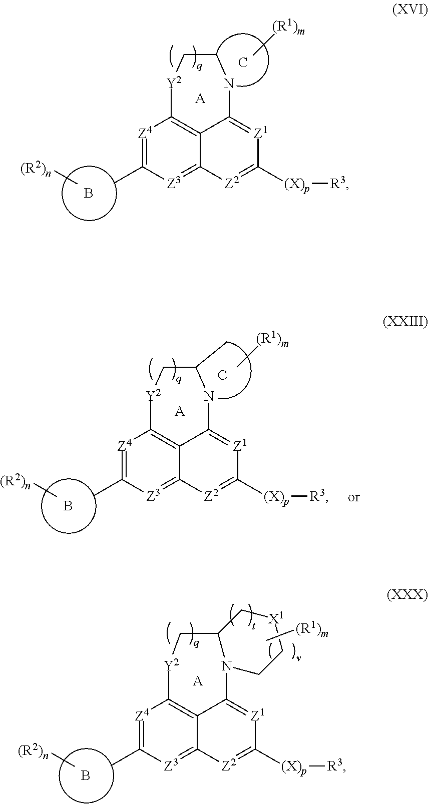

wherein ring C is a 4- to 8-membered heterocycloalkyl, X¹ is —NH—, —O— or —CH₂—, q is 0, 1, or 2, t is 1 or 2, and v is 1 or 2.

6. The compound of any one of clauses 1 to 5, or a pharmaceutically acceptable salt thereof, wherein the compound is of the formula IV, XIV, XVII, XXII, XXIV, XXIX, XXXI, or XXXVI,

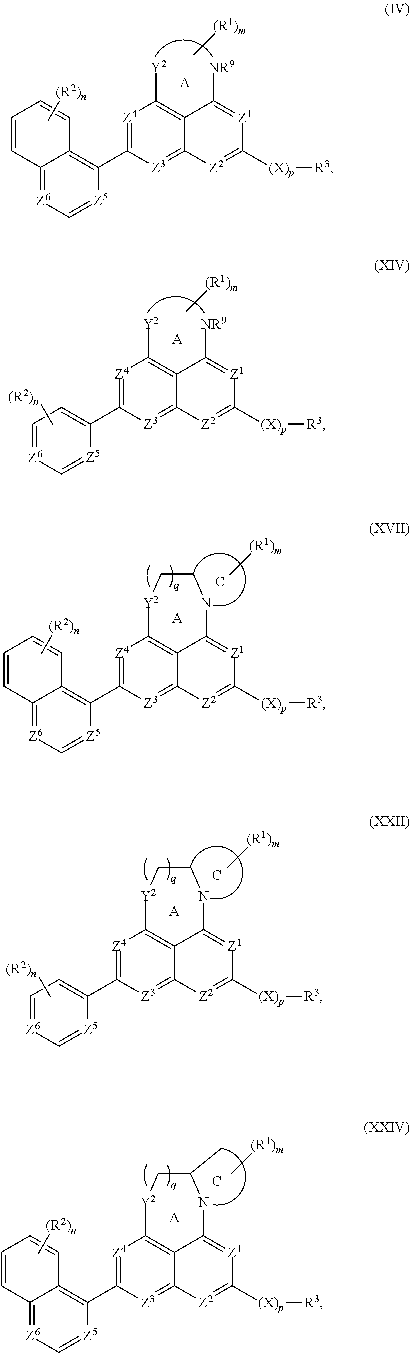

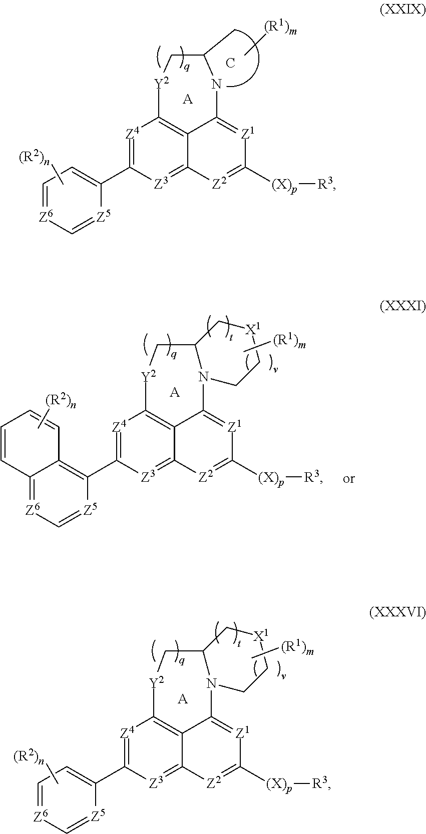

Z⁵ is N or C(R¹⁵);
Z⁶ is N or C(R¹⁶);
each of R¹⁵ and R¹⁶ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
ring C is a 4- to 8-membered heterocycloalkyl;
X¹ is —O—, —NH—, or —CH₂—;
q is 0, 1, or 2;
t is 1 or 2, and
v is 1 or 2.

7. The compound of any one of clauses 1 to 5, or a pharmaceutically acceptable salt thereof, wherein the compound is of the formula V, XI, XVIII, XXI, XXV, XXVIII, XXXII, or XXXV,

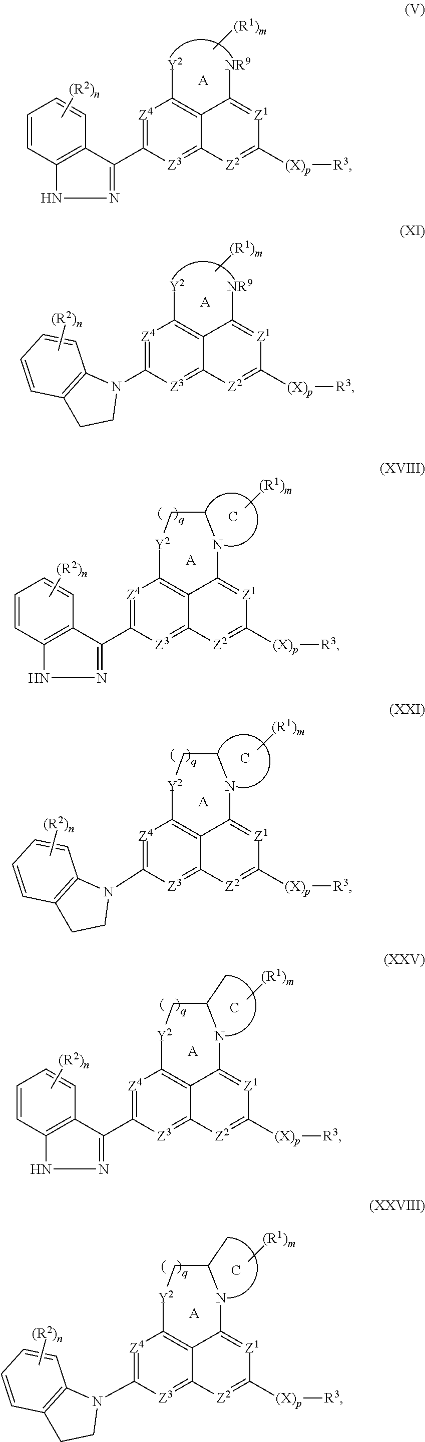

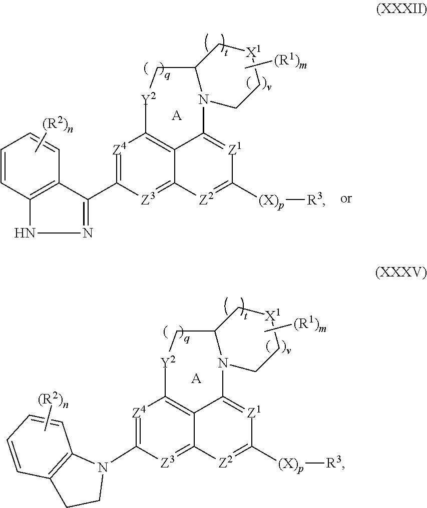

wherein ring C is a 4- to 8-membered heterocycloalkyl; X¹ is —O—, —NH—, or —CH₂—; q is 0, 1, or 2; t is 1 or 2; and v is 1 or 2.

8. The compound of any one of clauses 1 to 5, or a pharmaceutically acceptable salt thereof, wherein the compound is of the formula VI, X, XIX, XX, XXVI, XXVII, XXXIII, XXXIV, XXXXIII, XXXXIV, XXXXV, or XXXXVI,

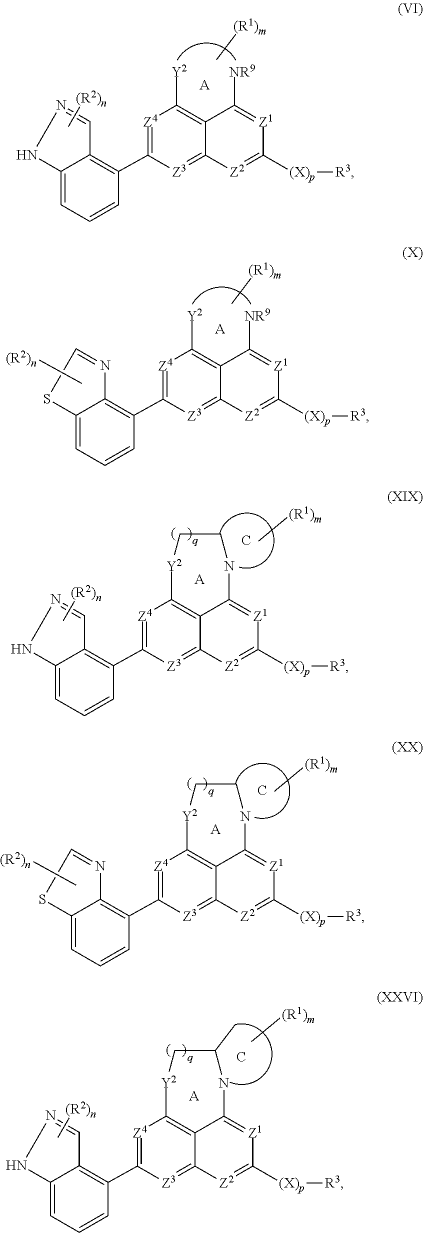

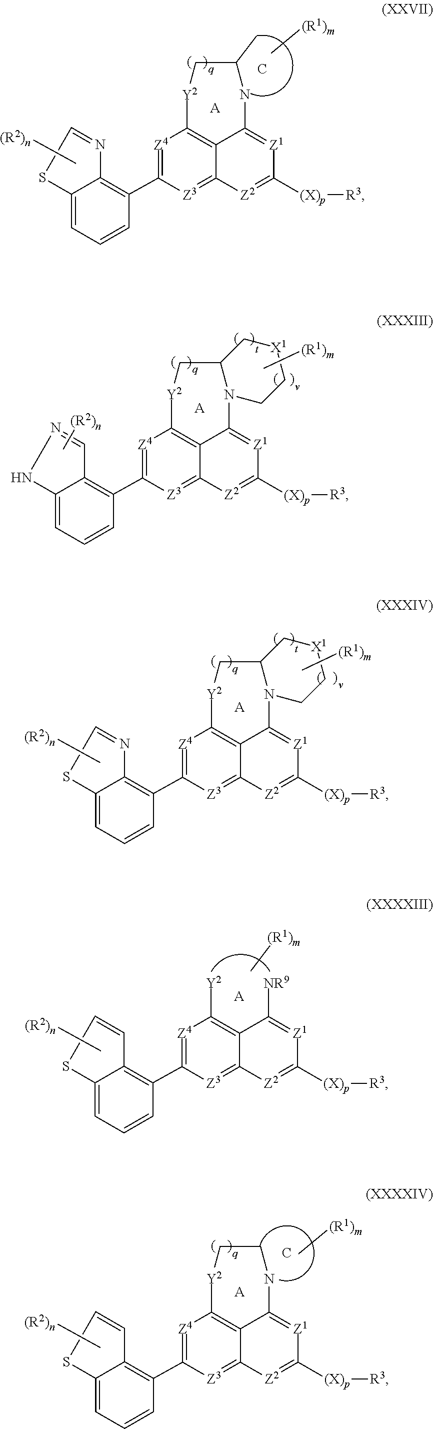

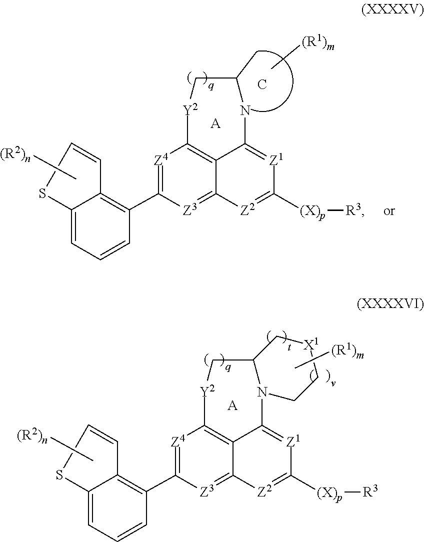

wherein ring C is a 4- to 8-membered heterocycloalkyl; X¹ is —O—, —NH— or —CH₂—; q is 0, 1, or 2; t is 1 or 2; and v is 1 or 2.

9. The compound of any one of clauses 1 to 5, or a pharmaceutically acceptable salt thereof, wherein the compound is of the formula VII or XV,

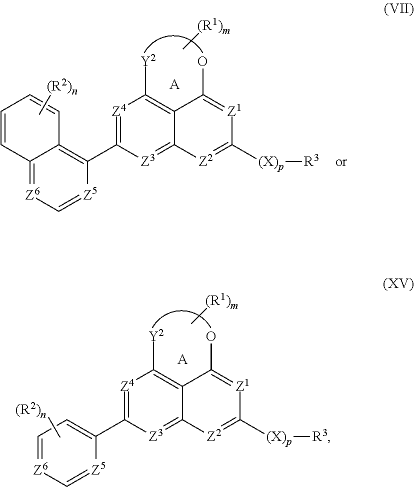

Z⁵ is N or C(R¹⁵);
Z⁶ is N or C(R¹⁶); and
each of R¹⁵ and R¹⁶ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂.

10. The compound of any one of clauses 1 to 5, or a pharmaceutically acceptable salt thereof, wherein the compound is of the formula VIII or XIII

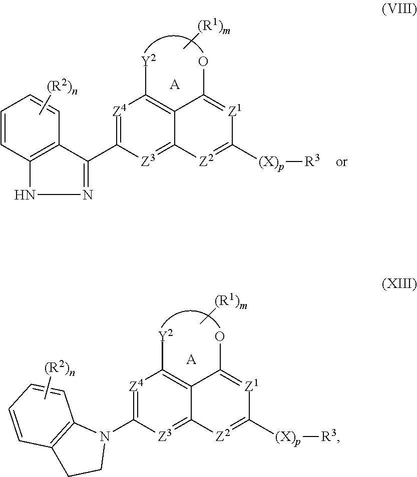

or a pharmaceutically acceptable salt thereof.

11. The compound of any one of clauses 1 to 5, or a pharmaceutically acceptable salt thereof, wherein the compound is of the formula IX or XII

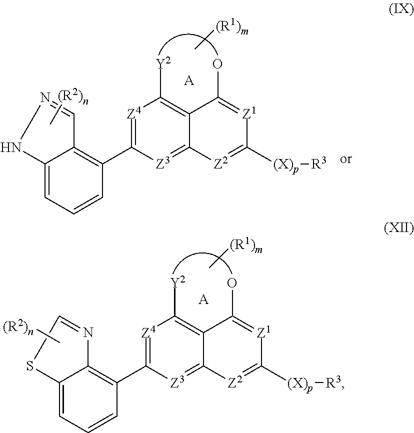

or a pharmaceutically acceptable salt thereof.

12. The compound of any one of the preceding clauses, or a pharmaceutically acceptable salt thereof, wherein R¹ is deuterium, C₁-C₆ alkyl, C₂-C₆ alkenyl, or C₂-C₆ alkynyl, wherein each hydrogen atom in C₁-C₆ alkyl, C₂-C₆ alkenyl, or C₂-C₆ alkynyl is independently optionally substituted by deuterium, halogen, —R^{e}, —R^{f}, C₁-C₆alkyl, C₁-C₆haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; R⁹ and a R¹ or R¹⁰ and a R¹, taken together with the atoms to which they are attached, combine to form a 4- to 10-membered heterocycloalkyl, wherein each hydrogen atom in the 4- to 10-membered heterocycloalkyl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂; or two hydrogen atoms on a single carbon atom of the 4- to 10-membered heterocycloalkyl combine to form an oxo group or an C₂-C₆ alkenyl group; or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

13. The compound of any one of the preceding clauses, or a pharmaceutically acceptable salt thereof, wherein ring A is optionally taken together with ring C or ring D to form a core of the compound of the formula I, the core having the formula

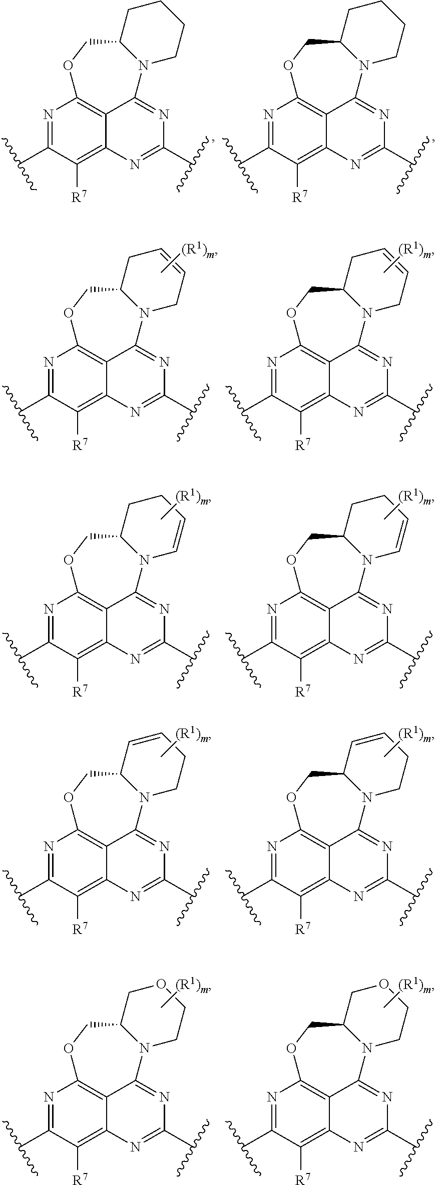

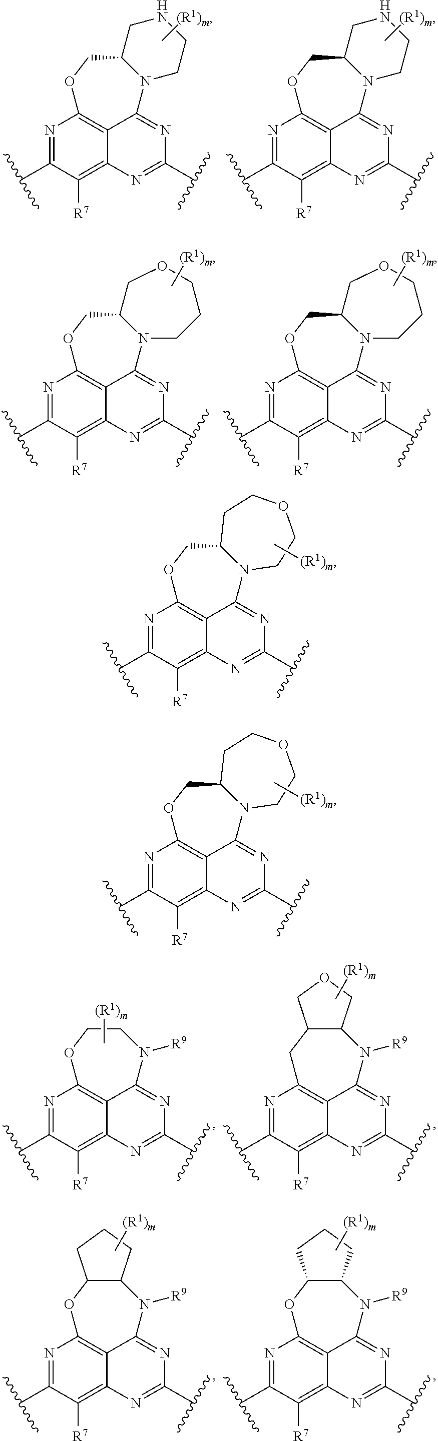

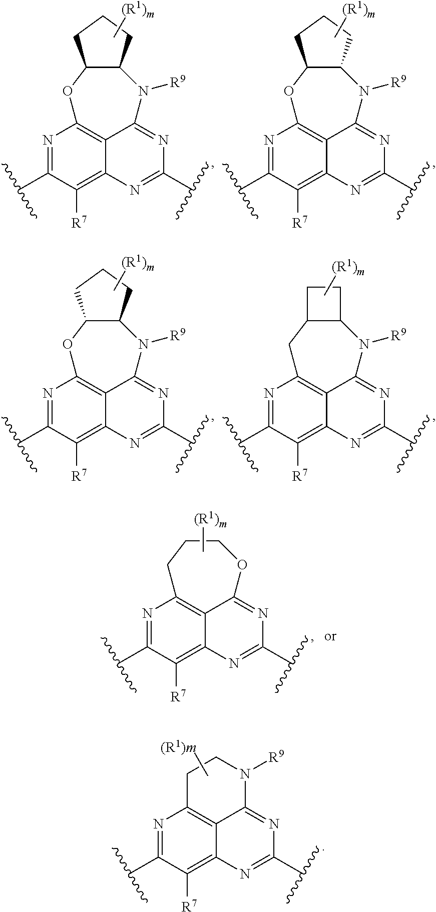

wherein each “ [symbol] ” is a point of covalent attachment to either ring B or —(X)ₚ—R³, and each hydrogen atom in ring A, ring C, if present, and ring D, if present, is independently optionally substituted with an R¹ selected from the group consisting of deuterium, halogen, C₁-C₆ alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{c}R^{f}, —OS(O)₂NR^{c}R^{f}, —SR^{c}, —S(O)R^{c}, —S(O)₂R^{c}, —S(O)NR^{c}R^{f}, —S(O)₂NR^{c}R^{f}, —NR^{c}R^{f}, —NR^{e}C(O)R^{f}, —NR^{e}C(O)OR^{f}, —NR^{e}C(O)NR^{e}R^{f}, —NR^{c}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

14. The compound of any one of the preceding clauses, or a pharmaceutically acceptable salt thereof, wherein ring A is optionally taken together with ring C or ring D to form core of the compound of the formula I, the core having the formula

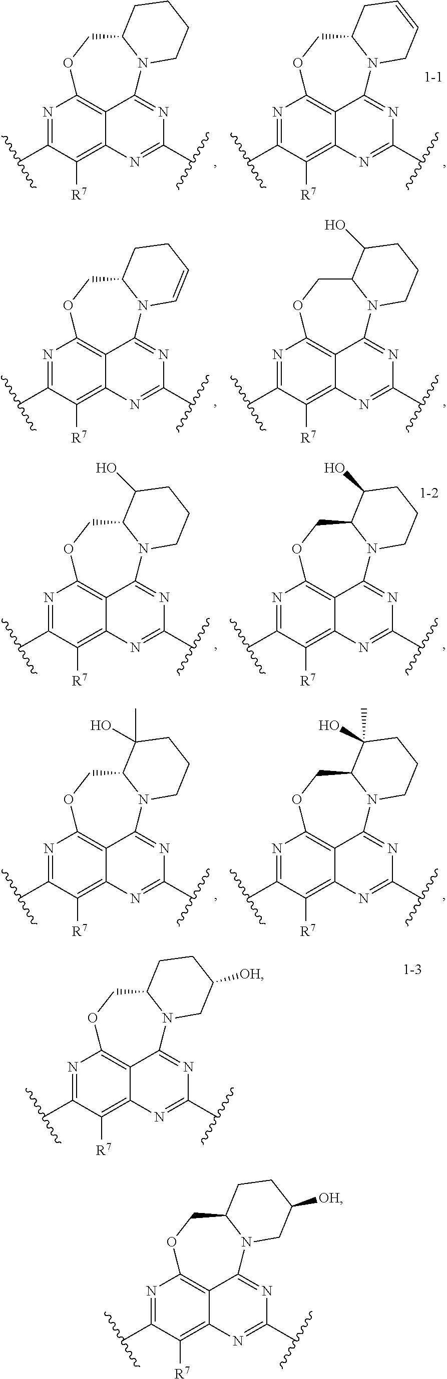

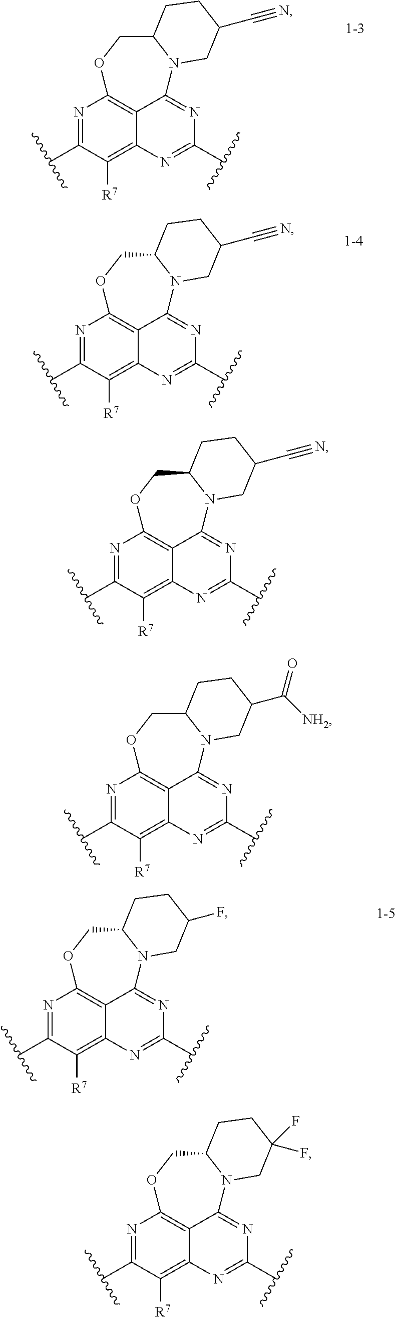

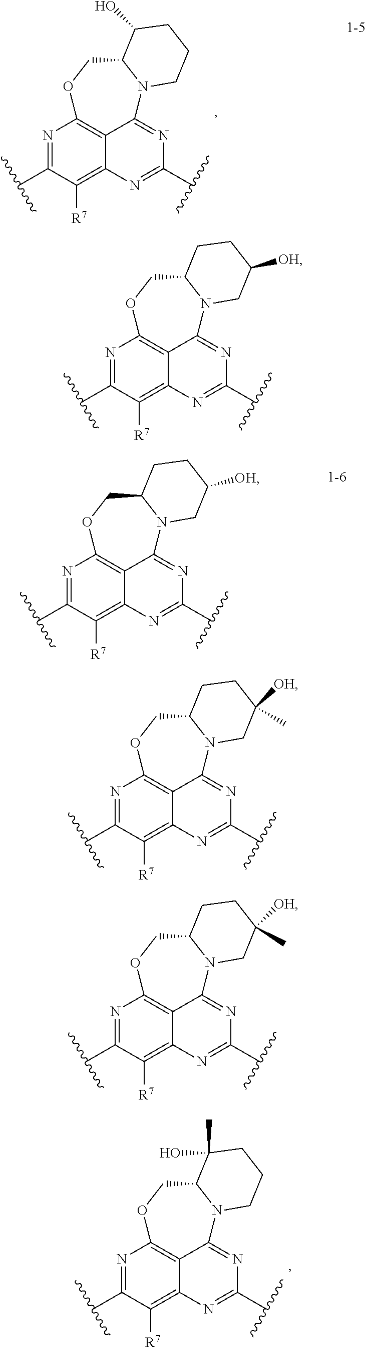

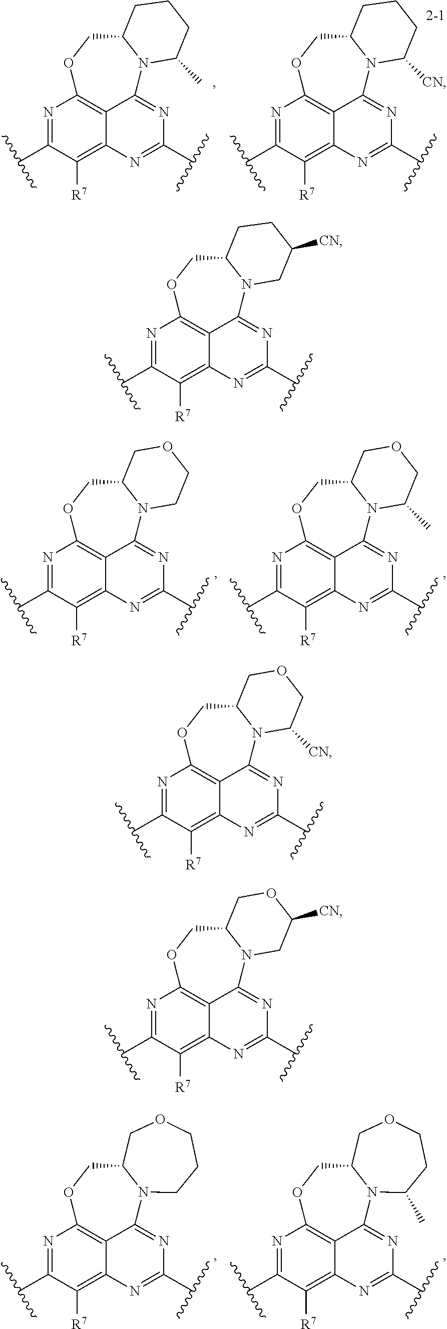

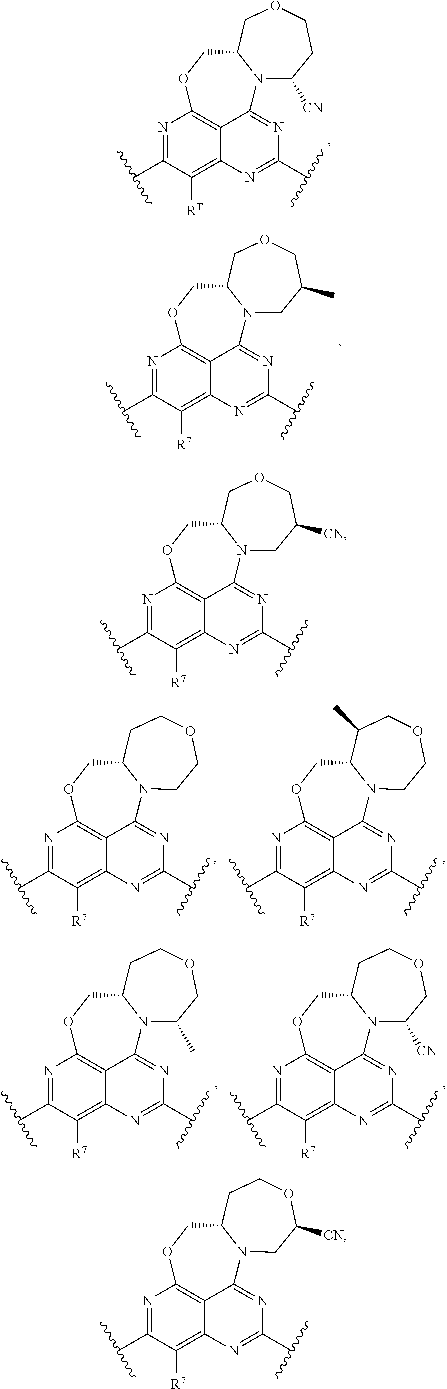

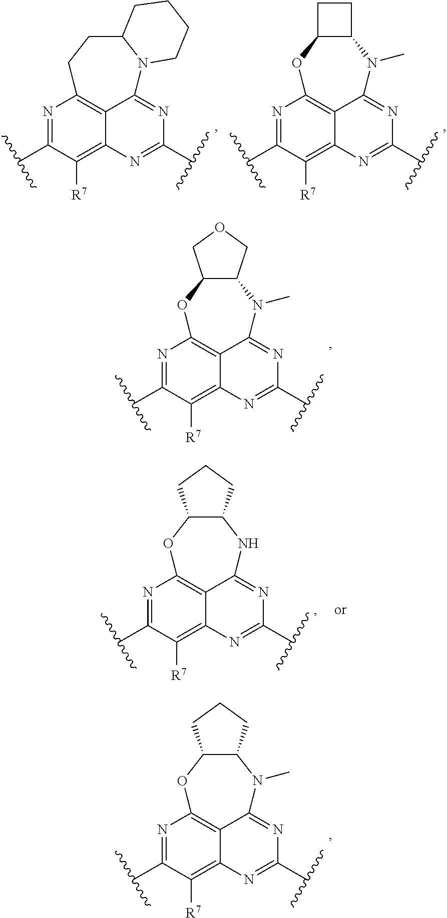

wherein each wherein “ [symbol] ” is a point of covalent attachment to either ring B or —Xₚ—R³.

15. The compound of any one of clauses 1 to 13, or a pharmaceutically acceptable salt thereof, wherein ring A is a fused 5- to 8-membered heterocycloalkyl that forms a core of the compound of the formula I, the core having the formula

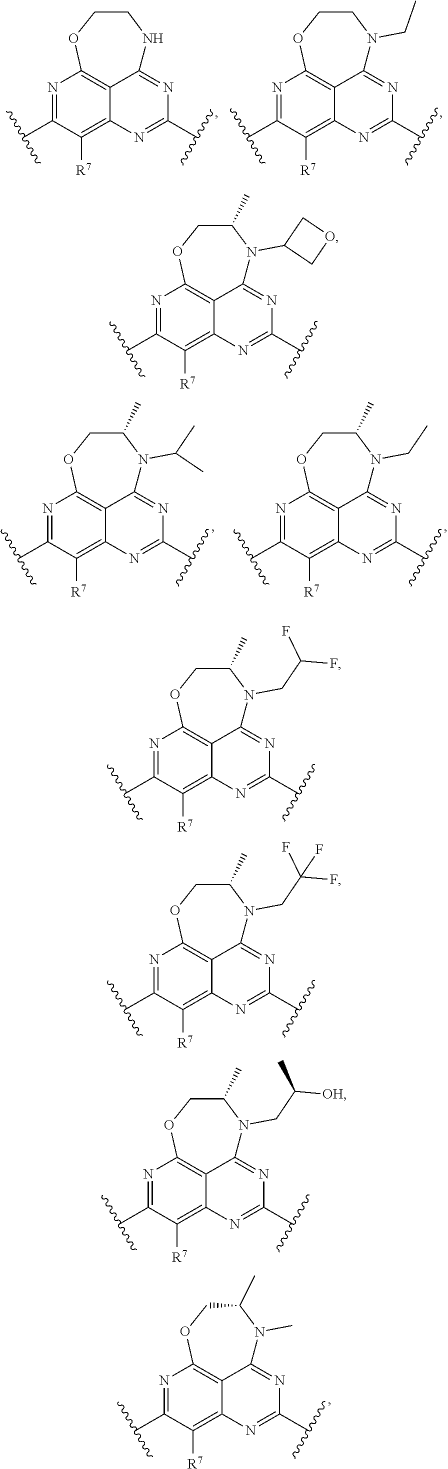

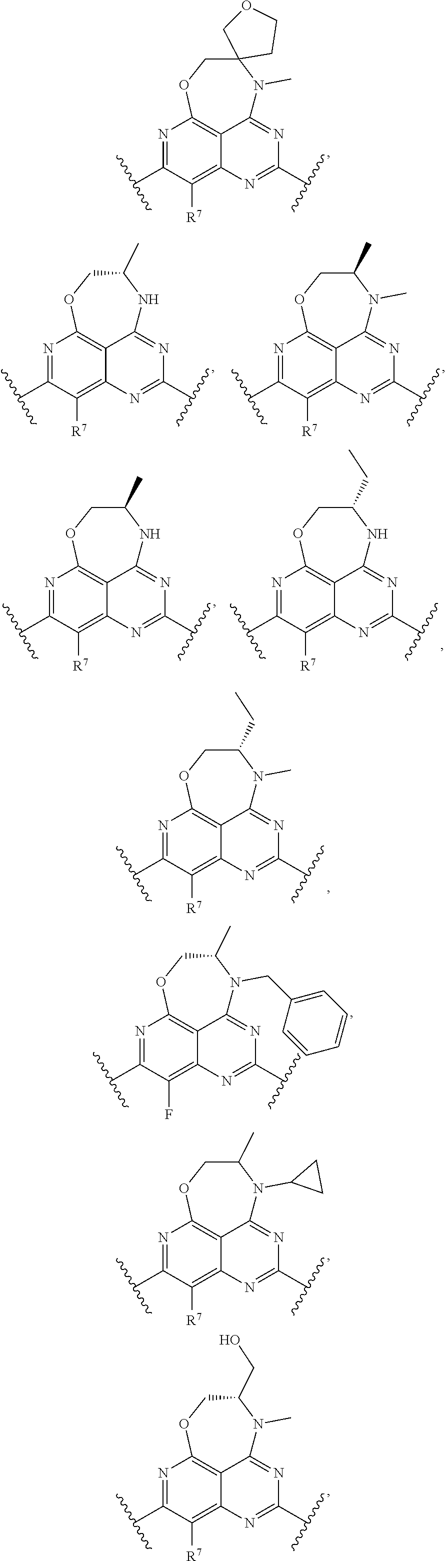

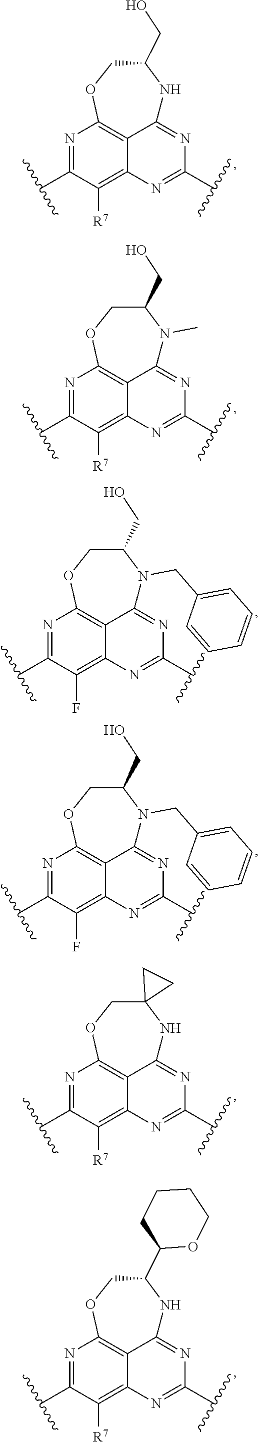

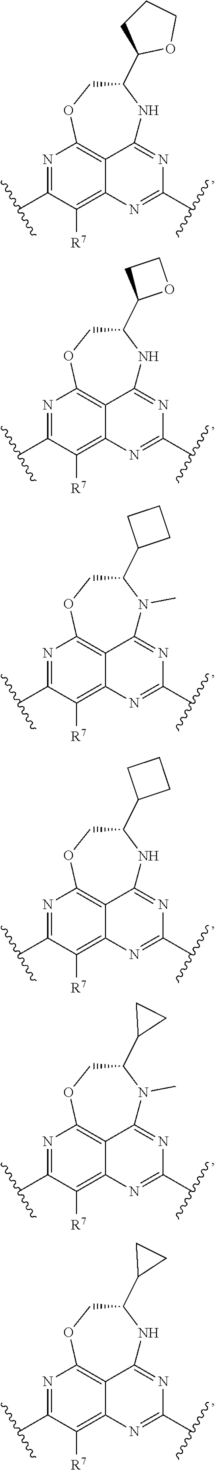

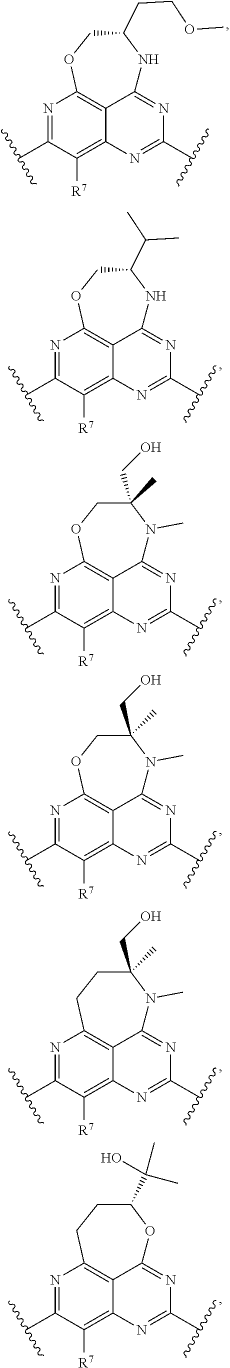

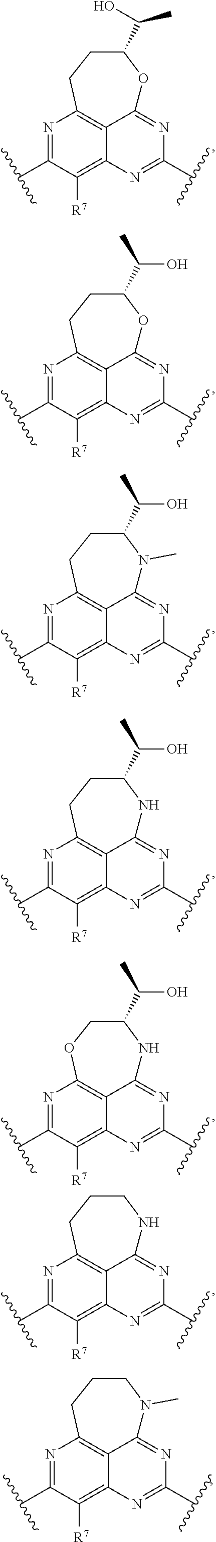

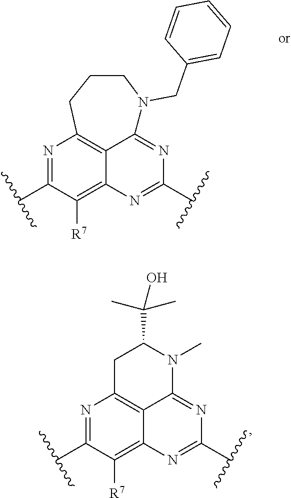

wherein each wherein “ [symbol] ” is a point of covalent attachment to either ring B or —(X)ₚ—R³.

16. The compound of any one of the preceding clauses, or a pharmaceutically acceptable salt thereof, wherein Y² is —O—.

17. The compound of any one of the preceding clauses, or a pharmaceutically acceptable salt thereof, wherein Y² is —CH₂—.

18. The compound of any one of the preceding clauses, or a pharmaceutically acceptable salt thereof, wherein R³ is —C₁-C₆ alkyl, 4- to 10-membered heterocycloalkyl, or —C₁-C₆ alkylene-(4- to 10-membered heterocycloalkyl), wherein each hydrogen atom in —C₁-C₆ alkyl, 4- to 10-membered heterocycloalkyl, or —C₁-C₆ alkylene-(4- to 10-membered heterocycloalkyl), is independently optionally substituted by deuterium, halogen, C₁-C₆ alkyl, —C₁-C₆alkylene-O—C₁-C₆ alkyl, —OC₁-C₆ alkylene-O—C₁-C₆alkyl, —C₁-C₆alkylene-O—R^{a}, C₆-C₁₀ aryl, —C₁-C₆alkylene-(C₆-C₁₀ aryl), haloalkyl, C₃-C₆cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

19. The compound of any one of the preceding clauses, or a pharmaceutically acceptable salt thereof, wherein R³ is —C₁-C₆ alkylene-(4- to 10-membered heterocycloalkyl), wherein each hydrogen atom in —C₁-C₆ alkylene-(4- to 10-membered heterocycloalkyl), is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

20. The compound of any one of the preceding clauses, or a pharmaceutically acceptable salt thereof, wherein R³ is

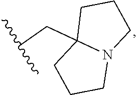 [SMILES: *CC12CCCN1CCC2]

wherein each hydrogen atom is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, and “ [symbol] ” is a point of covalent attachment.

21. The compound of any one of the preceding clauses, or a pharmaceutically acceptable salt thereof, wherein R³ is

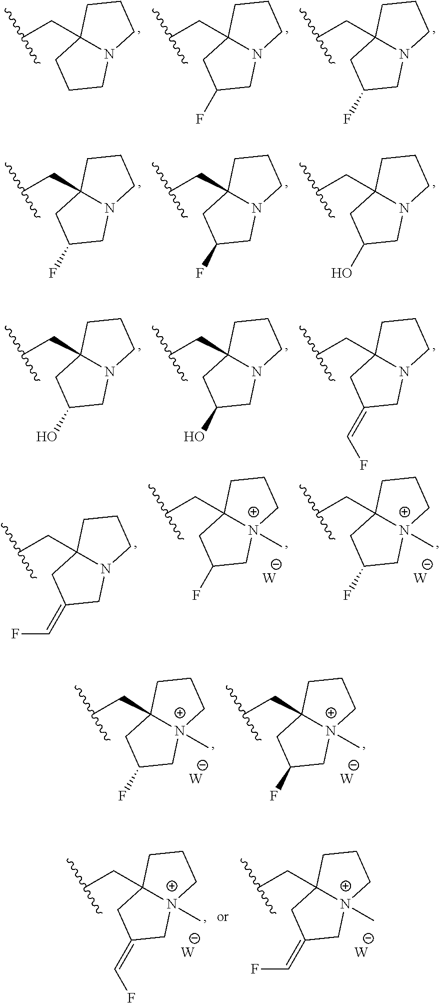

wherein W^{⊖} is an inorganic counter ion or an organic counter ion.

22. The compound of any one of clauses 1 to 18, or a pharmaceutically acceptable salt thereof, wherein R³ is 4- to 10-membered heterocycloalkyl, wherein each hydrogen atom in 4- to 10-membered heterocycloalkyl is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{c}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

23. The compound of any one of clauses 1 to 18, or 22, or a pharmaceutically acceptable salt thereof, wherein R³ is

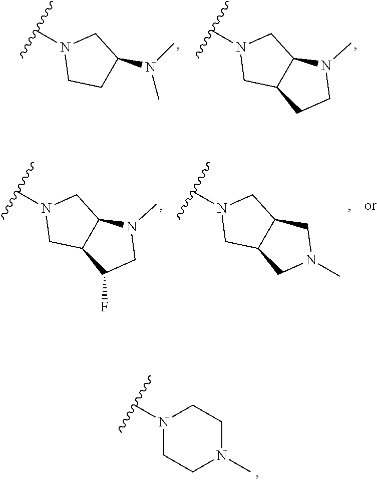

wherein “ [symbol] ” is a point of covalent attachment.

24. The compound of any one of the preceding clauses, or a pharmaceutically acceptable salt thereof, wherein m is 1, 2, 3, or 4.

25. The compound of any one of the preceding clauses, or a pharmaceutically acceptable salt thereof, wherein n is 0, 1, 2, or 3.

26. The compound of any one of the preceding clauses, or a pharmaceutically acceptable salt thereof, wherein Z¹ is N.

27. The compound of any one of the preceding clauses, or a pharmaceutically acceptable salt thereof, wherein Z² is N.

28. The compound of any one of the preceding clauses, or a pharmaceutically acceptable salt thereof, wherein Z³ is CR⁷ and Z⁴ is N.

29. The compound of any one of clauses 1 to 27, or a pharmaceutically acceptable salt thereof, wherein Z³ is CR⁷ and Z⁴ is CR⁸.

30. The compound of any one of clauses 1 to 27, or a pharmaceutically acceptable salt thereof, wherein Z³ is N and Z⁴ is CR⁸.

31. The compound of any one of clauses 1 to 27, or a pharmaceutically acceptable salt thereof, wherein Z³ is N and Z⁴ is N.

32. The compound of any one of clauses 1 to 27, or a pharmaceutically acceptable salt thereof, wherein Z³ is N, Z⁴ is CR⁸, Z⁵ is CR¹⁵, and Z⁶ is CR¹⁶.

33. The compound of any one of clauses 1 to 27, or a pharmaceutically acceptable salt thereof, wherein Z³ is CR⁷, Z⁴ is N, Z⁵ is CR¹⁵, and Z⁶ is CR¹⁶.

34. The compound of any one of clauses 1 to 27, or a pharmaceutically acceptable salt thereof, wherein Z³ is N, Z⁴ is N, Z⁵ is CR¹⁵, and Z⁶ is CR¹⁶.

35. The compound of any one of the preceding clauses, or a pharmaceutically acceptable salt thereof, wherein p is 0.

36. The compound of any one of clauses 1 to 34, or a pharmaceutically acceptable salt thereof, wherein p is 1.

37. The compound of any one of clauses 1 to 34, or 36, or a pharmaceutically acceptable salt thereof, wherein X is —O—.

38. The compound of any one of clauses 1 to 34, or 36, or a pharmaceutically acceptable salt thereof, wherein X is —NR⁴—.

39. The compound of any one of clauses 1 to 34, or 36, or a pharmaceutically acceptable salt thereof, wherein X is —S—.

40. The compound of any one of the preceding clauses, or a pharmaceutically acceptable salt thereof, wherein each R² is independently deuterium, halogen, C₁-C₆ alkyl, C₂-C₆ alkenyl, C₂-C₆ alkynyl, —OR^{c}, —C(O)R^{C}, —NR^{c}R^{d}, or —CN, wherein each hydrogen atom in C₁-C₆ alkyl, C₂-C₆ alkenyl, and C₂-C₆ alkynyl is independently optionally substituted with a deuterium or halogen.

41. The compound of any one of the preceding clauses, or a pharmaceutically acceptable salt thereof, wherein Ring B is

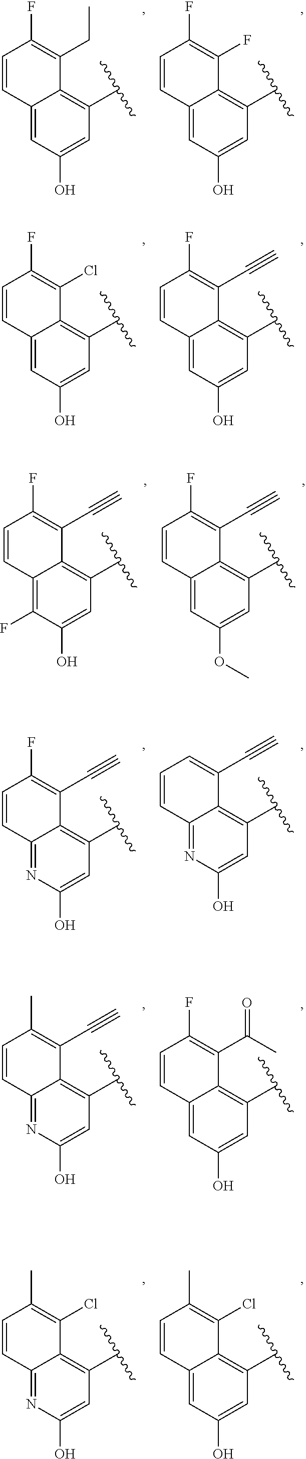

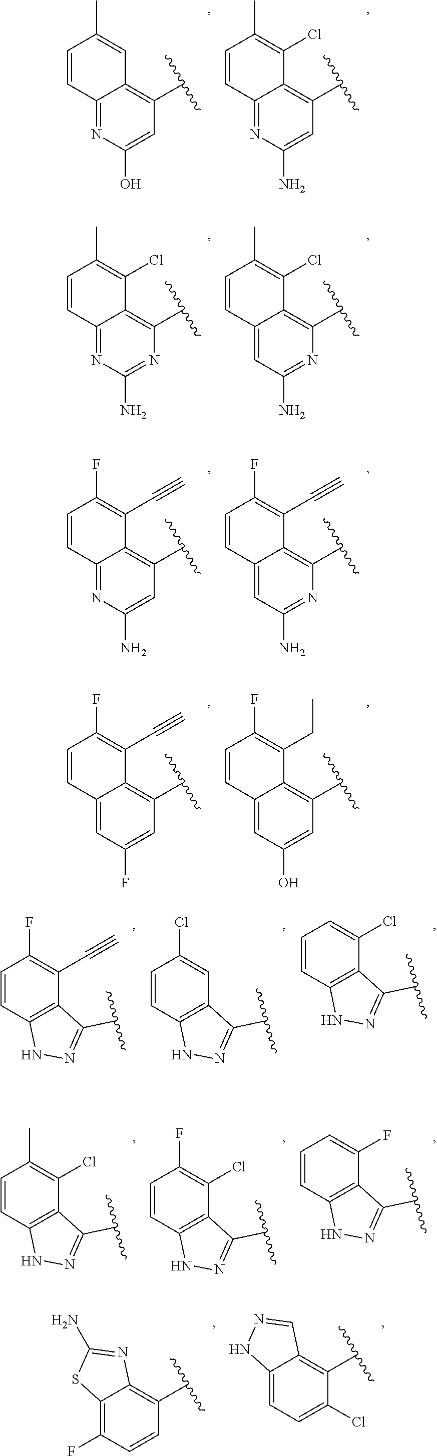

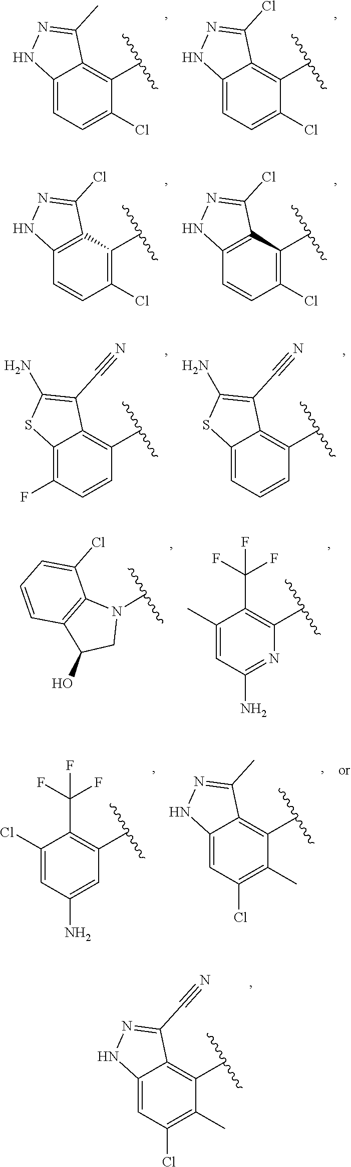

wherein “ [symbol] ” is a point of covalent attachment.

42. The compound of any one of the preceding clauses, or a pharmaceutically acceptable salt thereof, wherein R⁴, when present, is H or methyl.

43. The compound of any one of the preceding clauses, or a pharmaceutically acceptable salt thereof, wherein R⁵, when present, is H.

44. The compound of any one of the preceding clauses, or a pharmaceutically acceptable salt thereof, wherein R⁶, when present, is H.

45. The compound of any one of the preceding clauses, or a pharmaceutically acceptable salt thereof, wherein R⁷, when present, is H or F.

46. The compound of any one of the preceding clauses, or a pharmaceutically acceptable salt thereof, wherein R⁸, when present, is H.

47. The compound of any one of the preceding clauses, or a pharmaceutically acceptable salt thereof, wherein R⁹, when present, is H or methyl.

48. The compound of any one of the preceding clauses, or a pharmaceutically acceptable salt thereof, wherein R¹⁰, when present, is H or methyl.

49. The compound of any one of the preceding clauses, or a pharmaceutically acceptable salt thereof, wherein R¹¹, when present, is H.

50. The compound of any one of the preceding clauses, or a pharmaceutically acceptable salt thereof, wherein R¹², when present, is H.

51. The compound of any one of the preceding clauses, or a pharmaceutically acceptable salt thereof, wherein R¹³, when present, is H.

52. The compound of any one of the preceding clauses, or a pharmaceutically acceptable salt thereof, wherein R¹⁴, when present, is H.

53. The compound of any one of the preceding clauses, or a pharmaceutically acceptable salt thereof, wherein Z⁵, when present, is N.

54. The compound of any one of the preceding clauses, or a pharmaceutically acceptable salt thereof, wherein Z⁶, when present, is N.

55. The compound of any one of clauses 1 to 51, or a pharmaceutically acceptable salt thereof, wherein Z⁵, when present, is CR¹⁵.

56. The compound of any one of the preceding clauses, or a pharmaceutically acceptable salt thereof, wherein R¹⁵, when present, is H.

57. The compound of any one of clauses 1 to 53, or a pharmaceutically acceptable salt thereof, wherein Z⁶, when present, is CR¹⁶.

58. The compound of any one of the preceding clauses, or a pharmaceutically acceptable salt thereof, wherein R¹⁶, when present, is H.

59. The compound of clause 1, selected from the group consisting of 5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
5-ethyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
(8aR,9R)-5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7-oxa-1,3,6,12a-tetraazabenzo[4,5]cyclohepta[1,2,3-de]naphthalen-9-ol;
(8aR,9R)-5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7H-1,3,6,12a-tetraazabenzo[4,5]cyclohepta[1,2,3-de]naphthalen-9-ol;
(8aR,9R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol;
(8aR,9R)-5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol;
(8aS,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-11-ol;
(8aS,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-11-methyl-8,8a,9,10,11,12-hexahydro-7-oxa-1,3,6,12a-tetraazabenzo[4,5]cyclohepta[1,2,3-de]naphthalen-11-ol;
5-ethyl-6-fluoro-4-[(8S,8aS)-4-fluoro-2-{1[(2R,7aS)-2-fluorotetrahydro-1H-pyrrolizin-7a(5H)-yl]methoxy}-8-methyl-8,8a,9,10,11,12-hexahydro-7-oxa-1,3,6,12a-tetraazabenzo[4,5]cyclohepta[1,2,3-de]naphthalen-5-yl]naphthalen-2-ol;
7-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-1,3-benzothiazol-2-amine;
5-ethyl-6-fluoro-4-[(8aS)-4-fluoro-2-{[(2Z,7aS)-2-(fluoromethylidene)tetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
(3S)-7-chloro-1-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-2,3-dihydro-1H-indol-3-ol;
6-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-4-methyl-5-(trifluoromethyl)pyridin-2-amine;
3-chloro-5-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-4-(trifluoromethyl)aniline;
5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydropyrido[2′,1′:3,4][1,4]oxazepino[5,6,7-de]quinazolin-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-(propan-2-yl)-9,10-dihydro-8H-7-oxa-1,3,6,10-tetraazacyclohepta[1,2,3-de]naphthalen-5-yl]naphthalen-2-ol;
5-ethynyl-6-fluoro-4-[(8aS)-4-fluoro-2-{[(2Z,7aS)-2-(fluoromethylidene)tetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
(8aR,9R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7-oxa-1,3,6,12a-tetraazabenzo[4,5]cyclohepta[1,2,3-de]naphthalen-9-ol;
5-ethynyl-6-fluoro-4-[(7aR,10aS)-4-fluoro-2-{[(2R,7aS)-2-fluorotetrahydro-1H-pyrrolizin-7a(5H)-yl]methoxy}-11-methyl-7a,8,10a,11-tetrahydro-10H-7,9-dioxa-1,3,6,11-tetraazanaphtho[1,8-fg]azulen-5-yl]naphthalen-2-ol;
5-ethynyl-6-fluoro-4-[(7aS,9aS)-4-fluoro-2-{[(2R,7aS)-2-fluorotetrahydro-1H-pyrrolizin-7a(5H)-yl]methoxy}-10-methyl-8,9,9a,10-tetrahydro-7aH-7-oxa-1,3,6,10-tetraazacyclobuta[5,6]cyclohepta[1,2,3-de]naphthalen-5-yl]naphthalen-2-ol;
5-ethynyl-6-fluoro-4-[(8aS)-4-fluoro-2-{[(2R,7aS)-2-fluorotetrahydro-1H-pyrrolizin-7a(5H)-yl]methoxy}-8a,9,11,12-tetrahydro-8H-7,10-dioxa-1,3,6,12a-tetraazabenzo[4,5]cyclohepta[1,2,3-de]naphthalen-5-yl]naphthalen-2-ol;
5-ethynyl-6-fluoro-4-[(8aS,12S)-4-fluoro-2-{[(2R,7aS)-2-fluorotetrahydro-1H-pyrrolizin-7a(5H)-yl]methoxy}-12-methyl-8a,9,11,12-tetrahydro-8H-7,10-dioxa-1,3,6,12a-tetraazabenzo[4,5]cyclohepta[1,2,3-de]naphthalen-5-yl]naphthalen-2-ol;
(8aS,12S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8a,9,11,12-tetrahydro-8H-7,10-dioxa-1,3,6,12a-tetraazabenzo[4,5]cyclohepta[1,2,3-de]naphthalene-12-carbonitrile;
(8aS,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8a,9,11,12-tetrahydro-8H-7,10-dioxa-1,3,6,12a-tetraazabenzo[4,5]cyclohepta[1,2,3-de]naphthalene-11-carbonitrile;
(8aS,12R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-12-carbonitrile;
(8aS,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-11-carbonitrile;
5-ethynyl-6-fluoro-4-[(8aS)-4-fluoro-2-{[(2R,7aS)-2-fluorotetrahydro-1H-pyrrolizin-7a(5H)-yl]methoxy}-8a,9,12,13-tetrahydro-8H,11H-7,10-dioxa-1,3,6,13a-tetraazanaphtho[1,8-ab]heptalen-5-yl]naphthalen-2-ol;
5-ethynyl-6-fluoro-4-[(8aS,13S)-4-fluoro-2-{[(2R,7aS)-2-fluorotetrahydro-1H-pyrrolizin-7a(5H)-yl]methoxy}-13-methyl-8a,9,12,13-tetrahydro-8H,11H-7,10-dioxa-1,3,6,13a-tetraazanaphtho[1,8-ab]heptalen-5-yl]naphthalen-2-ol;
(8aS,13R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8a,9,12,13-tetrahydro-8H,11H-7,10-dioxa-1,3,6,13a-tetraazanaphtho[1,8-ab]heptalene-13-carbonitrile;
5-ethynyl-6-fluoro-4-[(8aS,12S)-4-fluoro-2-{1[(2R,7aS)-2-fluorotetrahydro-1H-pyrrolizin-7a(5H)-yl]methoxy}-12-methyl-8a,9,12,13-tetrahydro-8H,11H-7,10-dioxa-1,3,6,13a-tetraazanaphtho[1,8-ab]heptalen-5-yl]naphthalen-2-ol;
(8aS,12R)-5-(8-ethynyl-7-fluoro-3-hydroxynaphthalen-t-yl)-4-fluoro-2-{[(2R,7aS)-2-fluorotetrahydro-1H-pyrrolizin-7a(5H)-yl]methoxy}-8a,9,12,13-tetrahydro-8H,11H-7,10-dioxa-1,3,6,13a-tetraazanaphtho[1,8-ab]heptalene-12-carbonitrile;
5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10,12,13-hexahydro-7,11-dioxa-1,3,6,13a-tetraazanaphtho[1,8-ab]heptalen-5-yl]naphthalen-2-ol;
5-ethynyl-6-fluoro-4-[(8aS,9R)-4-fluoro-2-{[(2R,7aS)-2-fluorotetrahydro-1H-pyrrolizin-7a(5H)-yl]methoxy}-9-methyl-8,8a,9,10,12,13-hexahydro-7,11-dioxa-1,3,6,13a-tetraazanaphtho[1,8-ab]heptalen-5-yl]naphthalen-2-ol;
5-ethynyl-6-fluoro-4-[(8aS,13S)-4-fluoro-2-{[(2R,7aS)-2-fluorotetrahydro-1H-pyrrolizin-7a(5H)-yl]methoxy}-13-methyl-8,8a,9,10,12,13-hexahydro-7,11-dioxa-1,3,6,13a-tetraazanaphtho[1,8-ab]heptalen-5-yl]naphthalen-2-ol;
(8aS,13S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,12,13-hexahydro-7,11-dioxa-1,3,6,13a-tetraazanaphtho[1,8-ab]heptalene-13-carbonitrile;
(8aS,12R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,12,13-hexahydro-7,11-dioxa-1,3,6,13a-tetraazanaphtho[1,8-ab]heptalene-12-carbonitrile;
(8aS)-5-(5-chloro-1H-indazol-4-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene;
5-ethynyl-6-fluoro-4-[(8aS)-4-fluoro-2-{[(2E)-2-(fluoromethylidene)tetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
(5P,8aS)-5-(3,5-dichloro-1H-indazol-4-yl)-4-fluoro-2-{1[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene;
(5M,8aS)-5-(3,5-dichloro-1H-indazol-4-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene;
2-amino-7-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-1-benzothiophene-3-carbonitrile;
(8aS,11S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-11-ol;
(8aR,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-11-ol;
6-((S)-1-fluoro-11-(((2R,7aS)-2-fluorotetrahydro-1H-pyrrolizin-7a(5H)-yl)methoxy)-5,5a,6,7,8,9-hexahydro-4-oxa-3,9a,10,12-tetraazabenzo[4,5]cyclohepta[1,2,3-de]naphthalen-2-yl)-4-methyl-5-(trifluoromethyl)pyridin-2-amine;
(8aS)-5-(8-ethynyl-3,7-difluoronaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene;
(8aR,11S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-11-ol;
(2R,7aS)-7a-({[(8aS)-5-(8-ethynyl-7-fluoro-3-hydroxynaphthalen-1-yl)-4-fluoro-8,8a,9,10,11,12-hexahydro-7-oxa-1,3,6,12a-tetraazabenzo[4,5]cyclohepta[1,2,3-de]naphthalen-2-yl]oxy}methyl)hexahydro-1H-pyrrolizin-2-ol;
5-ethynyl-6-fluoro-4-[(8aS)-4,11,11-trifluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
(2S,7aS)-7a-({[(8aS)-5-(8-ethynyl-7-fluoro-3-hydroxynaphthalen-1-yl)-4-fluoro-8,8a,9,10,11,12-hexahydro-7-oxa-1,3,6,12a-tetraazabenzo[4,5]cyclohepta[1,2,3-de]naphthalen-2-yl]oxy}methyl)hexahydro-1H-pyrrolizin-2-ol;
5-ethynyl-6-fluoro-4-[(7aR,10aS)-4-fluoro-2-{[(2R,7aS)-2-fluorotetrahydro-1H-pyrrolizin-7a(5H)-yl]methoxy}-7a,8,9,10,10a,11-hexahydro-7-oxa-1,3,6,11-tetraazanaphtho[1,8-fg]azulen-5-yl]naphthalen-2-ol;
(8aS,9S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol;
5-ethynyl-6-fluoro-4-[4-fluoro-2-{[(2R,7aS)-2-fluorotetrahydro-1H-pyrrolizin-7a(5H)-yl]methoxy}-13-(hydroxymethyl)-8,9,10,11-tetrahydro-8,12-methano-7-oxa-1,3,6,12-tetraazacyclonona[1,2,3-de]naphthalen-5-yl]naphthalen-2-ol;
4-[(8aS)-4,11-difluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-5-ethynyl-6-fluoronaphthalen-2-ol;
5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,12-tetrahydro-7-oxa-1,3,6,12a-tetraazabenzo[4,5]cyclohepta[1,2,3-de]naphthalen-5-yl]naphthalen-2-ol;
5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10-tetrahydro-7-oxa-1,3,6,12a-tetraazabenzo[4,5]cyclohepta[1,2,3-de]naphthalen-5-yl]naphthalen-2-ol;
5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-11-carbonitrile;
5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-11-carboxamide;
(8aS,9S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7-oxa-1,3,6,12a-tetraazabenzo[4,5]cyclohepta[1,2,3-de]naphthalen-9-ol;
(8aS,9S)-5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7-oxa-1,3,6,12a-tetraazabenzo[4,5]cyclohepta[1,2,3-de]naphthalen-9-ol;
(8aS)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-11-carbonitrile;
5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol;
(8aS,11S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-11-methyl-8,8a,9,10,11,12-hexahydro-7-oxa-1,3,6,12a-tetraazabenzo[4,5]cyclohepta[1,2,3-de]naphthalen-11-ol; and
5-ethynyl-6-fluoro-4-(4-fluoro-2-{1[(2R,7aS)-2-fluorotetrahydro-1H-pyrrolizin-7a(5H)-yl]methoxy}-8,8a,9,10,11,12-hexahydro-7H-1,3,6,12a-tetraazabenzo[4,5]cyclohepta[1,2,3-de]naphthalen-5-yl)naphthalen-2-ol;
   or a pharmaceutically acceptable salt thereof.

60. The compound of clause 1, selected from the group consisting of 5-ethynyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-9,10-dimethyl-9,10-dihydro-8H-7-oxa-1,3,6,10-tetraazacyclohepta[1,2,3-de]naphthalen-5-yl]naphthalen-2-ol;
5-ethynyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-9,10-dimethyl-9,10-dihydro-8H-7-oxa-1,3,6,10-tetraazacyclohepta[1,2,3-de]naphthalen-5-yl]naphthalen-2-ol;
5-ethyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-9,10-dimethyl-9,10-dihydro-8H-7-oxa-1,3,6,10-tetraazacyclohepta[1,2,3-de]naphthalen-5-yl]naphthalen-2-ol;
5-ethynyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-9,10-dimethyl-7,8,9,10-tetrahydro-1,3,6,10-tetraazacyclohepta[1,2,3-de]naphthalen-5-yl]naphthalen-2-ol;
5-ethyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-9,10-dimethyl-7,8,9,10-tetrahydro-1,3,6,10-tetraazacyclohepta[1,2,3-de]naphthalen-5-yl]naphthalen-2-ol;
5-ethynyl-6-fluoro-4-[(9R)-4-fluoro-2-{[(2R,7aS)-2-fluorotetrahydro-1H-pyrrolizin-7a(5H)-yl]methoxy}-9-(2-hydroxypropan-2-yl)-8,9-dihydro-7H-10-oxa-1,3,6-triazacyclohepta[1,2,3-de]naphthalen-5-yl]naphthalen-2-ol;
5-ethyl-6-fluoro-4-[(9R)-4-fluoro-2-{[(2R,7aS)-2-fluorotetrahydro-1H-pyrrolizin-7a(5H)-yl]methoxy}-9-(2-hydroxypropan-2-yl)-8,9-dihydro-7H-10-oxa-1,3,6-triazacyclohepta[1,2,3-de]naphthalen-5-yl]naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(1S)-1-hydroxyethyl]-8,9-dihydro-7H-10-oxa-1,3,6-triazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1S)-1-hydroxyethyl]-8,9-dihydro-7H-10-oxa-1,3,6-triazacyclohepta[1,2,3-de]naphthalen-5-yl}naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-8,9-dihydro-7H-10-oxa-1,3,6-triazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-8,9-dihydro-7H-10-oxa-1,3,6-triazacyclohepta[1,2,3-de]naphthalen-5-yl}naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-10-methyl-7,8,9,10-tetrahydro-1,3,6,10-tetraazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-10-methyl-7,8,9,10-tetrahydro-1,3,6,10-tetraazacyclohepta[1,2,3-de]naphthalen-5-yl}naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-7,8,9,10-tetrahydro-1,3,6,10-tetraazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-7,8,9,10-tetrahydro-1,3,6,10-tetraazacyclohepta[1,2,3-de]naphthalen-5-yl}naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-9,10-dihydro-8H-7-oxa-1,3,6,10-tetraazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-9,10-dihydro-8H-7-oxa-1,3,6,10-tetraazacyclohepta[1,2,3-de]naphthalen-5-yl}naphthalen-2-ol;
5-ethynyl-6-fluoro-4-[(5R)-9-fluoro-2-{[(2R,7aS)-2-fluorotetrahydro-1H-pyrrolizin-7a(5H)-yl]methoxy}-5-(2-hydroxypropan-2-yl)-4-methyl-5,6-dihydro-4H-pyrimido[4,5,6-de][1,6]naphthyridin-8-yl]naphthalen-2-ol;
5-ethyl-6-fluoro-4-[(5R)-9-fluoro-2-{[(2R,7aS)-2-fluorotetrahydro-1H-pyrrolizin-7a(5H)-yl]methoxy}-5-(2-hydroxypropan-2-yl)-4-methyl-5,6-dihydro-4H-pyrimido[4,5,6-de][1,6]naphthyridin-8-yl]naphthalen-2-ol;
5-chloro-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-7,8,9,10-tetrahydro-1,3,6,10-tetraazacyclohepta[1,2,3-de]naphthalen-5-yl}naphthalen-2-ol;
5,6-difluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-7,8,9,10-tetrahydro-1,3,6,10-tetraazacyclohepta[1,2,3-de]naphthalen-5-yl}naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-10-methyl-9,10-dihydro-8H-7-oxa-1,3,6,10-tetraazacyclohepta[1,2,3-de]naphthalen-5-yl]naphthalen-2-ol;
5-ethyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-10-methyl-9,10-dihydro-8H-7-oxa-1,3,6,10-tetraazacyclohepta[1,2,3-de]naphthalen-5-yl]naphthalen-2-ol;
4-[(9S)-9-cyclopropyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethynyl-6-fluoronaphthalen-2-ol;
4-[(9S)-9-cyclopropyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-5-ethynyl-6-fluoronaphthalen-2-ol;
4-[(9S)-9-cyclopropyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethyl-6-fluoronaphthalen-2-ol;
4-[(9S)-9-cyclopropyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-5-ethyl-6-fluoronaphthalen-2-ol;
4-[(9S)-9-cyclobutyl-4-fluoro-2-{1[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethynyl-6-fluoronaphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(2R)-oxolan-2-yl]-9,10-dihydro-8H-7-oxa-1,3,6,10-tetraazacyclohepta[1,2,3-de]naphthalen-5-yl}naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(2R)-oxan-2-yl]-9,10-dihydro-8H-7-oxa-1,3,6,10-tetraazacyclohepta[1,2,3-de]naphthalen-5-yl}naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-methyl-9,10-dihydro-8H-7-oxa-1,3,6,10-tetraazacyclohepta[1,2,3-de]naphthalen-5-yl]naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(2R)-oxetan-2-yl]-9,10-dihydro-8H-7-oxa-1,3,6,10-tetraazacyclohepta[1,2,3-de]naphthalen-5-yl}naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-methyl-10-(propan-2-yl)-9,10-dihydro-8H-7-oxa-1,3,6,10-tetraazacyclohepta[1,2,3-de]naphthalen-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-methyl-10-(oxetan-3-yl)-9,10-dihydro-8H-7-oxa-1,3,6,10-tetraazacyclohepta[1,2,3-de]naphthalen-5-yl]naphthalen-2-ol;
5-ethynyl-6-fluoro-4-[(9S)-4-fluoro-2-{[(2R)-2-fluorotetrahydro-1H-pyrrolizin-7a(5H)-yl]methoxy}-9-(propan-2-yl)-9,10-dihydro-8H-7-oxa-1,3,6,10-tetraazacyclohepta[1,2,3-de]naphthalen-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-(2-methoxyethyl)-9,10-dihydro-8H-7-oxa-1,3,6,10-tetraazacyclohepta[1,2,3-de]naphthalen-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9,10-dimethyl-9,10-dihydro-8H-7-oxa-1,3,6,10-tetraazacyclohepta[1,2,3-de]naphthalen-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-10-methyl-9-(propan-2-yl)-9,10-dihydro-8H-7-oxa-1,3,6,10-tetraazacyclohepta[1,2,3-de]naphthalen-5-yl]naphthalen-2-ol;
4-[(9S)-10-benzyl-4-fluoro-2-{[(2R,7aS)-2-fluorotetrahydro-1H-pyrrolizin-7a(5H)-yl]methoxy}-9-methyl-9,10-dihydro-8H-7-oxa-1,3,6,10-tetraazacyclohepta[1,2,3-de]naphthalen-5-yl]-5-ethynyl-6-fluoronaphthalen-2-ol;
(2R,7aS)-7a-({[(9S)-5-(8-ethynyl-7-fluoro-3-hydroxynaphthalen-1-yl)-4-fluoro-9-methyl-9,10-dihydro-8H-7-oxa-1,3,6,10-tetraazacyclohepta[1,2,3-de]naphthalen-2-yl]oxy}methyl)-2-fluoro-4-methylhexahydro-1H-pyrrolizin-4-ium;
4-[(9S)-10-benzyl-4-fluoro-2-{[(2R,7aS)-2-fluorotetrahydro-1H-pyrrolizin-7a(5H)-yl]methoxy}-9-(hydroxymethyl)-9,10-dihydro-8H-7-oxa-1,3,6,10-tetraazacyclohepta[1,2,3-de]naphthalen-5-yl]-5-ethynyl-6-fluoronaphthalen-2-ol;
4-[(9R)-10-benzyl-4-fluoro-2-{[(2R,7aS)-2-fluorotetrahydro-1H-pyrrolizin-7a(5H)-yl]methoxy}-9-(hydroxymethyl)-9,10-dihydro-8H-7-oxa-1,3,6,10-tetraazacyclohepta[1,2,3-de]naphthalen-5-yl]-5-ethynyl-6-fluoronaphthalen-2-ol;
5-ethynyl-6-fluoro-4-(4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl)naphthalen-2-ol;
4-[(9S)-9-ethyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethynyl-6-fluoronaphthalen-2-ol;
5-ethynyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-10-methyl-9,10-dihydro-8H-7-oxa-1,3,6,10-tetraazacyclohepta[1,2,3-de]naphthalen-5-yl]naphthalen-2-ol;
5-ethyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-10-methyl-9,10-dihydro-8H-7-oxa-1,3,6,10-tetraazacyclohepta[1,2,3-de]naphthalen-5-yl]naphthalen-2-ol;
5-ethynyl-6-fluoro-4-(4′-fluoro-2′-{[(2R,7aS)-2-fluorotetrahydro-1H-pyrrolizin-7a(5H)-yl]methoxy}-10′-methyl-8′H,10′H-spiro[cyclopropane-1,9′-[7]oxa[1,3,6,10]tetraazacyclohepta[1,2,3-de]naphthalen]-5′-yl)naphthalen-2-ol;
5-ethynyl-6-fluoro-4-(4′-fluoro-2′-{[(2R,7aS)-2-fluorotetrahydro-1H-pyrrolizin-7a(5H)-yl]methoxy}-8′H,10′H-spiro[cyclopropane-1,9′-[7]oxa[1,3,6,10]tetraazacyclohepta[1,2,3-de]naphthalen]-5′-yl)naphthalen-2-ol;
4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-5-ethynyl-6-fluoronaphthalen-2-ol;
5-ethyl-4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-6-fluoronaphthalen-2-ol;
4-(10-ethyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl)-5-ethynyl-6-fluoronaphthalen-2-ol;
5-chloro-4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-6-fluoronaphthalen-2-ol;
4-(10-benzyl-4-fluoro-2-{[(2R,7aS)-2-fluorotetrahydro-1H-pyrrolizin-7a(5H)-yl]methoxy}-7,8,9,10-tetrahydro-1,3,6,10-tetraazacyclohepta[1,2,3-de]naphthalen-5-yl)-5-ethynyl-6-fluoronaphthalen-2-ol;
4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-5,6-difluoronaphthalen-2-ol;
2-amino-4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-1-benzothiophene-3-carbonitrile;
5-ethyl-6-fluoro-4-(4-fluoro-2-{[(2R,7aS)-2-fluorotetrahydro-1H-pyrrolizin-7a(5H)-yl]methoxy}-7,8,9,10-tetrahydro-1,3,6,10-tetraazacyclohepta[1,2,3-de]naphthalen-5-yl)naphthalen-2-ol;
5-ethynyl-6-fluoro-4-(4-fluoro-2-{[(2R,7aS)-2-fluorotetrahydro-1H-pyrrolizin-7a(5H)-yl]methoxy}-7,8,9,10-tetrahydro-1,3,6,10-tetraazacyclohepta[1,2,3-de]naphthalen-5-yl)naphthalen-2-ol;
5-ethyl-6-fluoro-4-[(9S)-4-fluoro-2-{[(2R,7aS)-2-fluorotetrahydro-1H-pyrrolizin-7a(5H)-yl]methoxy}-9,10-dimethyl-9,10-dihydro-8H-7-oxa-1,3,6,10-tetraazacyclohepta[1,2,3-de]naphthalen-5-yl]naphthalen-2-ol;
5-ethyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-9,10-dihydro-8H-7-oxa-1,3,6,10-tetraazacyclohepta[1,2,3-de]naphthalen-5-yl]naphthalen-2-ol;
1-[8-(10-cyclopropyl-4-fluoro-2-{[(2R,7aS)-2-fluorotetrahydro-1H-pyrrolizin-7a(5H)-yl]methoxy}-9-methyl-9,10-dihydro-8H-7-oxa-1,3,6,10-tetraazacyclohepta[1,2,3-de]naphthalen-5-yl)-2-fluoro-6-hydroxynaphthalen-1-yl]ethan-1-one;
4-(9-cyclopropyl-4-fluoro-2-(((2R,7aS)-2-fluorotetrahydro-1H-pyrrolizin-7a(5H)-yl)methoxy)-10-methyl-9,10-dihydro-8H-7-oxa-1,3,6,10-tetraazacyclohepta[de]naphthalen-5-yl)-5-ethynyl-6-fluoronaphthalen-2-ol;
4-(9-cyclopropyl-4-fluoro-2-(((2R,7aS)-2-fluorotetrahydro-1H-pyrrolizin-7a(5H)-yl)methoxy)-10-methyl-9,10-dihydro-8H-7-oxa-1,3,6,10-tetraazacyclohepta[de]naphthalen-5-yl)-5-ethynyl-6-fluoronaphthalen-2-ol;
5-ethyl-6-fluoro-4-((5aS)-1-fluoro-11-(((2R,7aS)-2-fluorotetrahydro-1H-pyrrolizin-7a(5H)-yl)methoxy)-5-methyl-5,5a,6,7,8,9-hexahydro-4-oxa-3,9a,10,12-tetraazabenzo[4,5]cyclohepta[1,2,3-de]naphthalen-2-yl)naphthalen-2-ol;
4-((S)-10-ethyl-4-fluoro-2-(((2R,7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-5-ethynyl-6-fluoronaphthalen-2-ol;
4-((S)-10-(2,2-difluoroethyl)-4-fluoro-2-(((2R,7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-5-ethynyl-6-fluoronaphthalen-2-ol; 5-ethynyl-6-fluoro-4-(4-fluoro-2-(((2R,7aS)-2-fluorotetrahydro-1H-pyrrolizin-7a(5H)-yl)methoxy)-10-methyl-4′,5′-dihydro-2′H,8H,10H-7-oxa-1,3,6,10-tetraazaspiro[cyclohepta[de]naphthalene-9,3′-furan]-1 (10a),2,3a,3a1 (6a),5-pentaen-5-yl)naphthalen-2-ol;
5-ethynyl-6-fluoro-4-((S)-4-fluoro-2-(((2R,7aS)-2-fluorotetrahydro-1H-pyrrolizin-7a(5H)-yl)methoxy)-10-((R)-2-hydroxypropyl)-9-methyl-9,10-dihydro-8H-7-oxa-1,3,6,10-tetraazacyclohepta[de]naphthalen-5-yl)naphthalen-2-ol;
4-((S)-10-(2,2-difluoroethyl)-4-fluoro-2-(((2R,7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-5-ethyl-6-fluoronaphthalen-2-ol;
5-ethyl-6-fluoro-4-(4-fluoro-2-(((2R,7aS)-2-fluorotetrahydro-1H-pyrrolizin-7a(5H)-yl)methoxy)-10-methyl-4′,5′-dihydro-2′H,8H,10H-7-oxa-1,3,6,10-tetraazaspiro[cyclohepta[de]naphthalene-9,3′-furan]-1 (10a),2,3a,3a1 (6a),5-pentaen-5-yl)naphthalen-2-ol; and
5-ethynyl-6-fluoro-4-((S)-4-fluoro-2-(((2R,7aS)-2-fluorotetrahydro-1H-pyrrolizin-7a(5H)-yl)methoxy)-9-methyl-10-(2,2,2-trifluoroethyl)-9,10-dihydro-8H-7-oxa-1,3,6,10-tetraazacyclohepta[de]naphthalen-5-yl)naphthalen-2-ol;
   or a pharmaceutically acceptable salt thereof.

61. A pharmaceutical composition comprising at least one compound of any one of clauses 1 to 60, or a pharmaceutically acceptable salt thereof, and optionally one or more pharmaceutically acceptable excipients.

62. A method of treating disease, such as cancer, comprising administering to a subject in need of such treatment an effective amount of a compound of any one of clauses 1 to 60, or a pharmaceutically acceptable salt thereof.

63. A compound of any one of clauses 1 to 60, or a pharmaceutically acceptable salt thereof, for use in a method of treating cancer in a subject.

64. A compound of any one of clauses 1 to 60, or a pharmaceutically acceptable salt thereof, for treating cancer in a subject.

65. Use of a compound of any one of clauses 1 to 60, or a pharmaceutically acceptable salt thereof, in the manufacture of a medicament for treating cancer in a subject.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended clauses.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended clauses, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “**”, and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by “A-*” “A-**”, or

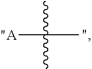

where each of “-*”, “-**” and

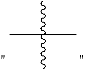

represents a bond to A and the point of covalent bond attachment to B. Alternatively, in some embodiments, the portion of A-B defined by the group or chemical structure B can be represented by “*—B”, “**—B”, or

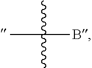

where each of “-*”, “-**”, and

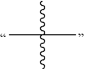

represents a bond to B and the point of covalent bond attachment to A.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Chemical Definitions

The term “alkyl” refers to a straight- or branched-chain monovalent hydrocarbon group. The term “alkylene” refers to a straight- or branched-chain divalent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. It will be appreciated that an alkyl or alkylene group can be unsubstituted or substituted as described herein. An alkyl or alkylene group can be substituted with any of the substituents in the various embodiments described herein, including one or more of such substituents.

The term “alkenyl” refers to a straight- or branched-chain mono-valent hydrocarbon group having one or more double bonds. In some embodiments, it can be advantageous to limit the number of atoms in an “alkenyl” to a specific range of atoms, such as C₂-C₂₀ alkenyl, C₂-C₁₂ alkenyl, or C₂-C₆ alkenyl. Examples of alkenyl groups include ethenyl (or vinyl), allyl, and but-3-en-1-yl. Included within this term are cis and trans isomers and mixtures thereof. It will be appreciated that an alkenyl can be unsubstituted or substituted as described herein. An alkenyl group can be substituted with any of the substituents in the various embodiments described herein, including one or more of such substituents.

The term “alkynyl” refers to a straight- or branched-chain monovalent hydrocarbon group having one or more triple bonds. In some embodiments, it can be advantageous to limit the number of atoms in an “alkynyl” to a specific range of atoms, such as C₂-C₂₀ alkynyl, C₂-C₁₂ alkynyl, or C₂-C₆ alkynyl. Examples of alkynyl groups include acetylenyl (—C≡CH) and propargyl (—CH₂C≡CH), but-3-yn-1,4-diyl (—C≡C—CH₂CH₂—), and the like. It will be appreciated that an alkynyl group can be unsubstituted or substituted as described herein. An alkynyl group can be substituted with any of the substituents in the various embodiments described herein, including one or more of such substituents.

The term “cycloalkyl” refers to a saturated or partially saturated, monocyclic or polycyclic mono-valent carbocycle. In some embodiments, it can be advantageous to limit the number of atoms in a “cycloalkyl” to a specific range of atoms, such as having 3 to 12 ring atoms. Polycyclic carbocycles include fused, bridged, and spiro polycyclic systems. Illustrative examples of cycloalkyl groups include monovalent radicals of the following entities:

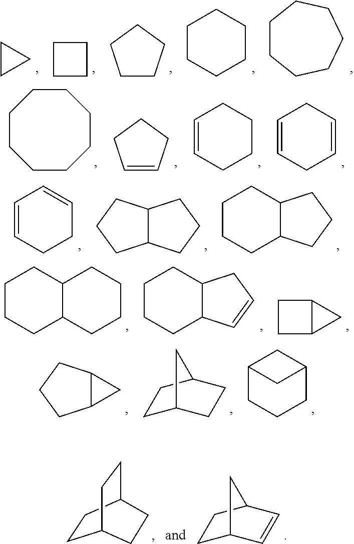

In particular, a cyclopropyl moiety can be depicted by the structural formula

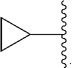 [SMILES: *C1CC1]

It will be appreciated that a cycloalkyl group can be unsubstituted or substituted as described herein. A cycloalkyl group can be substituted with any of the substituents in the various embodiments described herein, including one or more of such substituents.

The term “halogen” or “halo” represents chlorine, fluorine, bromine, or iodine.

The term “haloalkyl” refers to an alkyl group with one or more halo substituents. Examples of haloalkyl groups include —CF₃, —(CH₂)F, —CHF₂, —CH₂Br, —CH₂CF₃, and —CH₂CH₂F.

In certain embodiments, alkenyl may be substituted. For example, the alkenyl may be substituted with halo (e.g., fluoro). Illustratively, the alkenyl substituted with halo may be a ═CHF. Illustratively, a hexahydro-1H-pyrrolizinyl moiety may be substituted by halo-substituted alkenyl, for example, a substituted hexahydro-1H-pyrrolizinyl moiety can be depicted by the structural formula

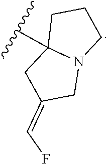 [SMILES: *C12CCCN1C/C(=C\F)C2]

The term “aryl” refers to a monovalent all-carbon monocyclic or fused-ring polycyclic group having a completely conjugated pi-electron system. In some embodiments, it can be advantageous to limit the number of atoms in an “aryl” to a specific range of atoms, such as mono-valent all-carbon monocyclic or fused-ring polycyclic groups of 6 to 14 carbon atoms (C₆-C₁₄ aryl), or monovalent all-carbon monocyclic or fused-ring polycyclic groups of 6 to 10 carbon atoms (C₆-C₁₀ aryl). Examples, without limitation, of aryl groups are phenyl, naphthalenyl and anthracenyl. It will be appreciated that an aryl group can be unsubstituted or substituted as described herein. An aryl group can be substituted with any of the substituents in the various embodiments described herein, including one or more of such substituents.

The term “heterocycloalkyl” refers to a mono-valent monocyclic or polycyclic ring structure that is saturated or partially saturated having one or more non-carbon ring atoms. In some embodiments, it can be advantageous to limit the number of atoms in a “heterocycloalkyl” to a specific range of ring atoms, such as from 3 to 12 ring atoms (3- to 12-membered), or 3 to 7 ring atoms (3- to 7-membered), or 3 to 6 ring atoms (3- to 6-membered), or 4 to 6 ring atoms (4- to 6-membered), 5 to 7 ring atoms (5- to 7-membered), or 4 to 10 ring atoms (4- to 10-membered). In some embodiments, it can be advantageous to limit the number and type of ring heteroatoms in “heterocycloalkyl” or to a specific range or type of heteroatoms, such as 1 to 5 ring heteroatoms selected from nitrogen, oxygen, and sulfur. Examples, without limitations, of mono-cyclic heterocycloalkyl groups include tetrahydrofuran, pyrrolidine, and morpholine. Polycyclic ring systems include fused, bridged, and spiro systems. In some embodiments, it can be advantageous to limit the number of atoms in a bicyclic “heterocycloalkyl” to a specific range of ring atoms, such as from 5 to 10 ring atoms (5- to 10-membered), or 6 to 10 ring atoms (6- to 10-membered). The ring structure may optionally contain an oxo group or an imino group on a carbon ring member or up to two oxo groups on sulfur ring members. Examples, without limitations, of fused bicyclic, bridged bicyclic, and spiro bicyclic heterocycloalkyl groups include pyrrolizine, 2,5-diazabicyclo[2.2.2]octane, and 1-oxaspiro[4.5]decane. Illustrative examples of heterocycloalkyl groups include monovalent radicals of the following entities:

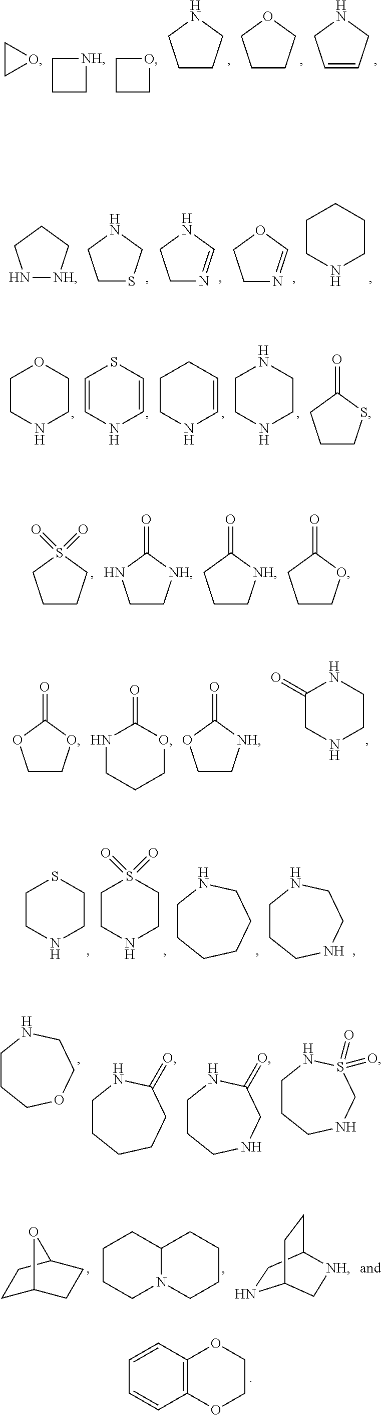

A three-membered heterocycle may contain at least one heteroatom ring atom, where the heteroatom ring atom is a sulfur, oxygen, or nitrogen. Non-limiting examples of three-membered heterocycle groups include monovalent and divalent radicals of oxirane, azetidine, and thiirane. A four-membered heterocycle may contain at least one heteroatom ring atom, where the heteroatom ring atom is a sulfur, oxygen, or nitrogen. Non-limiting examples of four-membered heterocycle groups include monovalent and divalent radicals of azitidine, oxtenane, and thietane. A five-membered heterocycle can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of five-membered heterocyle groups include mono-valent and divalent radicals of pyrrolidine, tetrahydrofuran, 2, 5-dihydro-1H-pyrrole, pyrazolidine, thiazolidine, 4,5-dihydro-1H-imidazole, dihydrothiophen-2 (3H)-one, tetrahydrothiophene 1,1-dioxide, imidazolidin-2-one, pyrrolidin-2-one, dihydrofuran-2 (3H)-one, 1,3-dioxolan-2-one, and oxazolidin-2-one. A six-membered heterocycle can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of six-membered heterocycle groups include mono-valent or divalent radicals of piperidine, morpholine, 4H-1,4-thiazine, 1,2,3,4-tetrahydropyridine, piperazine, 1,3-oxazinan-2-one, piperazin-2-one, thiomorpholine, and thiomorpholine 1,1-dioxide. A “heterobicycle” is a fused bicyclic system comprising one heterocycle ring fused to a cycloalkyl or another heterocycle ring.

In particular, a hexahydro-1H-pyrrolizinyl moiety can be depicted by the structural formula

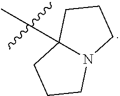 [SMILES: *C12CCCN1CCC2]

It will be appreciated that a heterocycloalkyl group can be unsubstituted or substituted as described herein. A heterocycloalkyl group can be substituted with any of the substituents in the various embodiments described herein, including one or more of such substituents.

The term “heteroaryl” refers to a mono-valent monocyclic, fused bicyclic, or fused polycyclic aromatic heterocycle (ring structure having ring atoms or members selected from carbon atoms and up to four heteroatoms selected from nitrogen, oxygen, and sulfur) that is fully unsaturated and having from 3 to 12 ring atoms per heterocycle. The term “heteroarylene” refers to a divalent monocyclic, fused bicyclic, or fused polycyclic aromatic heterocycle (ring structure having ring atoms or members selected from carbon atoms and up to four heteroatoms selected from nitrogen, oxygen, and sulfur) having from 3 to 12 ring atoms per heterocycle. In some embodiments, it can be advantageous to limit the number of ring atoms in a “heteroaryl” or “heteroarylene” to a specific range of atom members, such as 5- to 10-membered heteroaryl. In some instances, a 5- to 10-membered heteroaryl can be a monocyclic ring or fused bicyclic rings having 5- to 10-ring atoms wherein at least one ring atom is a heteroatom, such as N, O, or S. The ring structure may optionally contain an oxo group or an imino group on a carbon ring member or up to two oxo groups on sulfur ring members. Illustrative examples of 5- to 10-membered heteroaryl groups include monovalent radicals of the following entities, while examples of 5- to 10-membered heteroarylene groups include divalent radicals of the following entities, in the form of properly bonded moieties:

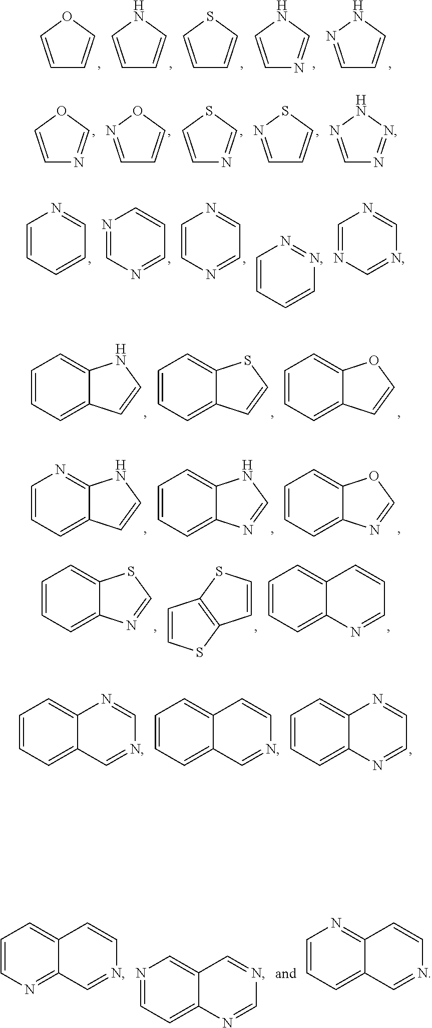

In some embodiments, a “monocyclic” heteroaryl can be an aromatic five- or six-membered heterocycle. A five-membered heteroaryl can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of five-membered heteroaryl groups include mono-valent radicals of furan, thiophene, pyrrole, oxazole, isoxazole, thiazole, isothiazole, pyrazole, imidazole, oxadiazole, thiadiazole, triazole, or tetrazole. A six-membered heteroaryl can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of six-membered heteroaryl groups include monovalent radicals of pyridine, pyrazine, pyrimidine, pyridazine, or triazine. A “bicyclic heteroaryl” is a fused bicyclic system comprising one heteroaryl ring fused to a phenyl or another heteroaryl ring. Non-limiting examples of bicyclic heteroaryl groups include monovalent radicals of quinoline, isoquinoline, quinazoline, quinoxaline, 1,5-naphthyridine, 1,8-naphthyridine, isoquinolin-3 (2H)-one, thieno[3,2-b]thiophene, 1H-pyrrolo[2,3-b]pyridine, indazole, 1H-benzo[d]imidazole, benzo[d]oxazole, and benzo[d]thiazole.

In particular, an isoquinolin-3 (2H)-onyl moiety can be depicted by the structural formula

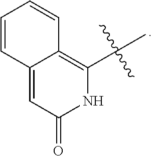 [SMILES: *c1[nH]c(=O)cc2ccccc12]

In particular, a divalent pyridopyrimidine moiety can be depicted by the structural formula

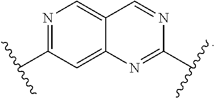 [SMILES: *c1cc2nc(*)ncc2cn1]

It will be appreciated that a heteroaryl group can be unsubstituted or substituted as described herein. A heteroaryl group can be substituted with any of the substituents in the various embodiments described herein, including one or more of such substituents.

It will be appreciated that a heteroaryl or heteroarylene group can be unsubstituted or substituted as described herein. A heteroaryl or heteroarylene group can be substituted with any of the substituents in the various embodiments described herein, including one or more of such substituents.

The term “oxo” represents a carbonyl oxygen. For example, a cyclopentyl substituted with oxo is cyclopentanone.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Certain chemical entities of Formula (I)—(XXXXVI) may be depicted in two or more tautomeric forms. Any and all alternative tautomers are included within the scope of these formulas, and no inference should be made as to whether the chemical entity exists as the tautomeric form in which it is drawn. It will be understood that the chemical entities described herein, and their constituent rings A, B, etc. can exist in different tautomeric forms. It will be readily appreciated by one of skill in the art that because of rapid interconversion, tautomers can generally be considered to be the same chemical compound. Examples of tautomers include but are not limited to enol-keto tautomers, amine-imine tutomers, and the like.

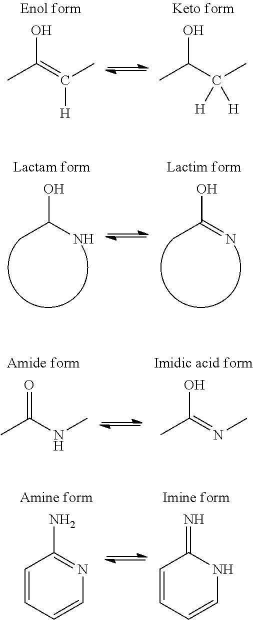

In particular, a ring option of isoquinolin-3 (2H)-oneylene can exist as the following tautomers

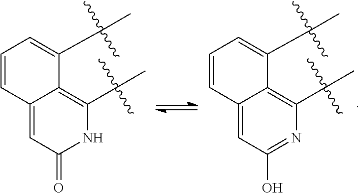 [SMILES: *c1cccc2cc(=O)[nH]c(*)c12 ; *c1cccc2cc(O)nc(*)c12]

The nomenclature “(ATOM)ᵢ-(ATOM)ⱼ” with j>i, when applied herein to a class of substituents, is meant to refer to embodiments of this disclosure for which each and every one of the number of atom members, from i to j including i and j, is independently realized. By way of example, the term C₁-C₃ refers independently to embodiments that have one carbon member (C₁), embodiments that have two carbon members (C₂), and embodiments that have three carbon members (C₃).

The disclosure also includes pharmaceutically acceptable salts of the compounds represented by Formula (I)—(XXXXVI), preferably of those described above and of the specific compounds exemplified herein, and pharmaceutical compositions comprising such salts, and methods of using such salts.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. ⊖,” wherein “W^{⊖}” is an inorganic counter ion (e.g., an inorganic anion) or an organic counter ion (e.g., an organic anion). In certain embodiments, W^{⊖} is an anion that is complexed with a cation of a compound of the disclosure to form a pharmaceutically acceptable salt.

It will be understood that the chemical entities described herein, can exist as a salt of a free acid or base of a compound represented herein and an inorganic or organic counter ion. Illustratively, the salt can be formed during the manufacture of the compound (e.g., a salt or a pharmaceutically acceptable salt) or can substituted to a salt for further manufacture, formulation, or administration reasons. As illustrated herein, certain compounds include a “W ⊖,” wherein “W^{⊖}” is an inorganic counter ion (e.g., an inorganic anion) or an organic counter ion (e.g., an organic anion). In certain embodiments, W^{⊖} is an anion that is complexed with a cation of a compound of the disclosure to form a pharmaceutically acceptable salt.

The term “inorganic counter ion” represents an inorganic ion that accompanies an ionic species in order to maintain electric neutrality. An inorganic counter ion may represent an anion or cation. An inorganic counterion may accompany a free acid or base of a compound represented herein. An inorganic ion may form by a reaction of an inorganic base or inorganic acid and a compound described herein that possesses a sufficiently acidic group, a sufficiently basic group, both types of functional groups, or more than one of each type.

The term “organic counter ion” represents an organic counter ion that accompanies an ionic species in order to maintain electric neutrality. The organic ion may represent an anion or cation. An organic counter ion may accompany a free acid or base of a compound represented herein. An organic ion may form by a reaction of an organic base or organic acid and a compound described herein that possesses a sufficiently acidic group, a sufficiently basic group, both types of functional groups, or more than one of each type.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula (I)—(XXXXVI) that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The disclosure also relates to pharmaceutically acceptable prodrugs of the compounds of Formula (I)—(XXXXVI), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)—(XXXXVI)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs,” ed. H. Bundgaard, Elsevier, 1985.

The present disclosure also relates to pharmaceutically active metabolites of compounds of Formula (I)—(XXXXVI), and uses of such metabolites in the methods of the disclosure. A “pharmaceutically active metabolite” means a pharmacologically active product of metabolism in the body of a compound of Formula (I)—(XXXXVI) or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

As used herein, the term “KRAS inhibitor” includes, but is not limited to, a compound that is capable of inhibiting the protein encoded by the KRAS gene, called K-Ras, that is involved in the RAS/MAPK signaling pathway. The terms KRAS gene, K-Ras, and RAS/MAPK signaling pathway will be known and understood by one of skill in the art. It will be appreciated that KRAS mutations occur in approximately one in seven of all human metastatic cancers, and that those mutations can occur in a variety of locations in the KRAS gene coding sequence. KRAS mutations primarily occur in KRAS codons 12 and 13, and also occur in codons 18, 61, 117, and 146 at low frequencies and have distinct effects on tumor cell signaling based on the codon and missense mutation. Examples of KRAS mutations include, but are not limited to KRAS G12C, KRAS G12D, KRAS G12V, KRAS G12R, KRAS G12S, KRAS G13C, KRAS G13D, KRAS A18D, KRAS Q61H, KRAS K117N, and the like. It will be understood by a person having ordinary skill in the art that reference to a inhibiting of KRAS mutations, such as KRAS G12D refers to inhibiting the protein encoded by the KRAS G12D gene, having a coding sequence (e.g. a guanine to adenine substitution, at position 35 on codon 12 of the KRAS coding sequence) that produces a K-Ras G12D protein, where a glycine at position 12 of the protein sequence is replaced by am aspartic acid.

### Representative Embodiments

In some embodiments, the disclosure relates to a compound of the formula I, or a pharmaceutically acceptable salt thereof,

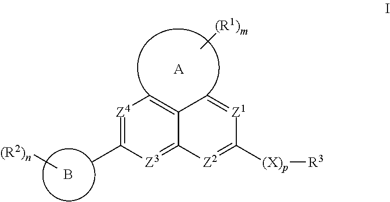

wherein R¹, R², R³, R⁹, A, B, X, Y², Z¹, Z², Z³, Z⁴, m, n, and p are as described herein.

In some embodiments, the disclosure provides a compound of the formula II, or a pharmaceutically acceptable salt thereof,

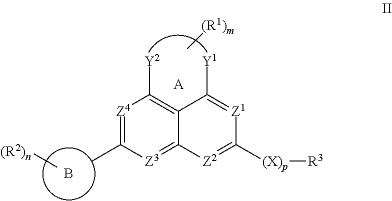

wherein R¹, R², R³, A, B, X, Y¹, Y², Z¹, Z², Z³, Z⁴, m, n, and p are as described herein.

In some embodiments, the disclosure provides a compound of the formula III, or a pharmaceutically acceptable salt thereof,

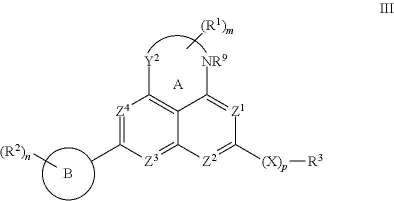

wherein R¹, R², R³, R⁹, A, B, X, Y², Z¹, Z², Z³, Z⁴, m, n, and p are as described herein.

In some embodiments, the disclosure provides a compound of the formula IV, or a pharmaceutically acceptable salt thereof,

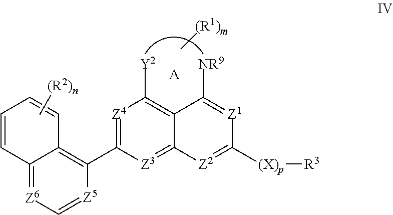

wherein R¹, R², R³, R⁹, A, X, Y², Z¹, Z², Z³, Z⁴, Z⁵, Z⁶, m, n, and p are as described herein.

In some embodiments, the disclosure provides a compound of the formula IV, or a pharmaceutically acceptable salt thereof,

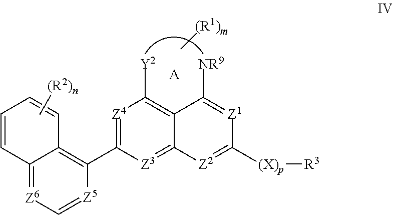

wherein R¹, R², R³, R⁹, A, X, Y², Z¹, Z², Z³, Z⁴, m, n, and p are as described herein, and Z⁵ is N or C(R¹⁵), and Z⁶ is N or C(R¹⁶).

In some embodiments, the disclosure provides a compound of the formula V, or a pharmaceutically acceptable salt thereof,

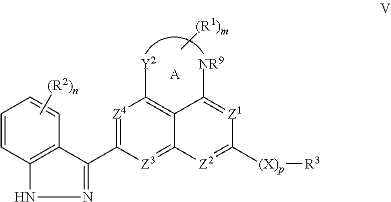

wherein R¹, R², R³, R⁹, A, X, Y², Z¹, Z², Z³, Z⁴, m, n, and p are as described herein.

In some embodiments, the disclosure provides a compound of the formula VI, or a pharmaceutically acceptable salt thereof,

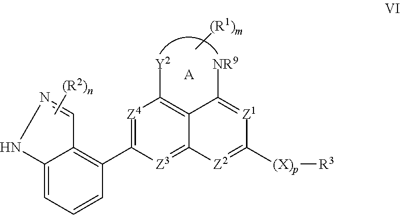

wherein R¹, R², R³, R⁹, A, X, Y², Z¹, Z², Z³, Z⁴, m, n, and p are as described herein.

In some embodiments, the disclosure provides a compound of the formula VII, or a pharmaceutically acceptable salt thereof,

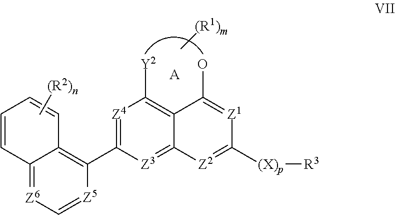

wherein R¹, R², R³, A, X, Y², Z¹, Z², Z³, Z⁴, Z⁵, Z⁶, m, n, and p are as described herein.

In some embodiments, the disclosure provides a compound of the formula VII, or a pharmaceutically acceptable salt thereof,

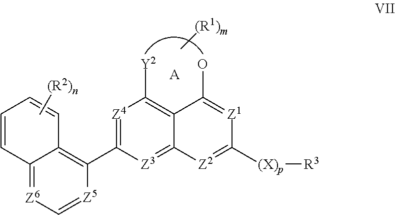

wherein R¹, R², R³, A, X, Y², Z¹, Z², Z³, Z⁴, m, n, and p are as described herein, and Z⁵ is N or C(R¹⁵), and Z⁶ is N or C(R¹⁶)

In some embodiments, the disclosure provides a compound of the formula VIII, or a pharmaceutically acceptable salt thereof,

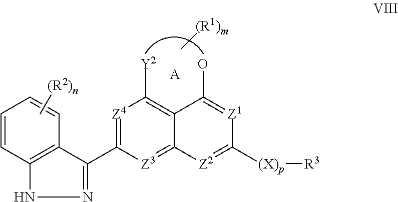

wherein R¹, R², R³, A, X, Y², Z¹, Z², Z³, Z⁴, m, n, and p are as described herein.

In some embodiments, the disclosure provides a compound of the formula IX, or a pharmaceutically acceptable salt thereof,

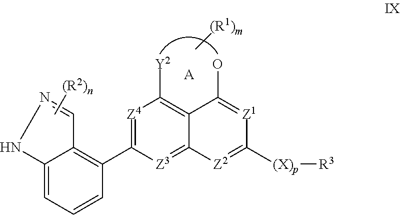

wherein R¹, R², R³, A, X, Y², Z¹, Z², Z³, Z⁴, m, n, and p are as described herein.

In some embodiments, the disclosure provides a compound of the formula X, or a pharmaceutically acceptable salt thereof,

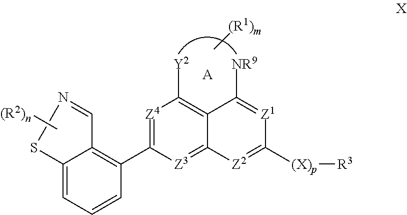

wherein R¹, R², R³, R⁹, A, X, Y², Z¹, Z², Z³, Z⁴, m, n, and p are as described herein.

In some embodiments, the disclosure provides a compound of the formula XI, or a pharmaceutically acceptable salt thereof,

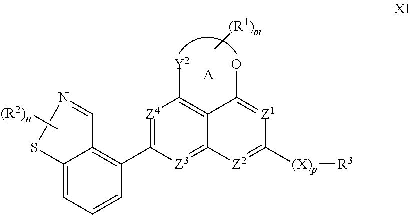

wherein R¹, R², R³, A, X, Y², Z¹, Z², Z³, Z⁴, m, n, and p are as described herein.

In some embodiments, the disclosure provides a compound of the formula XII, or a pharmaceutically acceptable salt thereof,

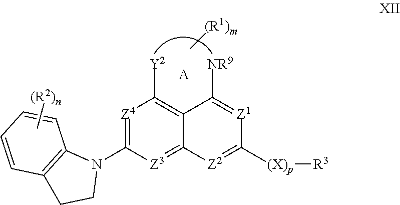

wherein R¹, R², R³, R⁹, A, X, Y², Z¹, Z², Z³, Z⁴, m, n, and p are as described herein.

In some embodiments, the disclosure provides a compound of the formula XIII, or a pharmaceutically acceptable salt thereof,

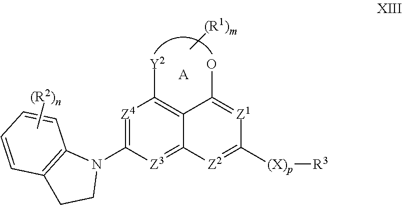

wherein R¹, R², R³, A, X, Y², Z¹, Z², Z³, Z⁴, m, n, and p are as described herein.

In some embodiments, the disclosure provides a compound of the formula XIV, or a pharmaceutically acceptable salt thereof,

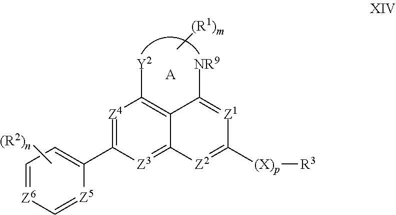

wherein R¹, R², R³, R⁹, A, X, Y², Z¹, Z², Z³, Z⁴, Z⁵, Z⁶, m, n, and p are as described herein.

In some embodiments, the disclosure provides a compound of the formula XIV, or a pharmaceutically acceptable salt thereof,

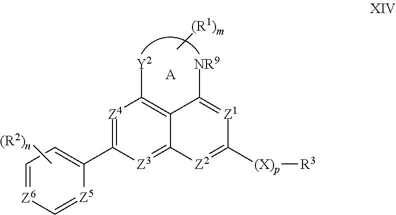

wherein R¹, R², R³, R⁹, A, X, Y², Z¹, Z², Z³, Z⁴, m, n, and p are as described herein; Z⁵ is N or C(R¹⁵); and Z⁶ is N or C(R¹⁶)

In some embodiments, the disclosure provides a compound of the formula XV, or a pharmaceutically acceptable salt thereof,

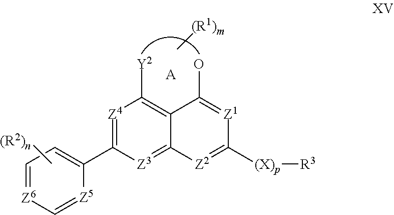

wherein R¹, R², R³, R⁹, A, X, Y², Z¹, Z², Z³, Z⁴, Z⁵, Z⁶, m, n, and p are as described herein.

In some embodiments, the disclosure provides a compound of the formula XV, or a pharmaceutically acceptable salt thereof,

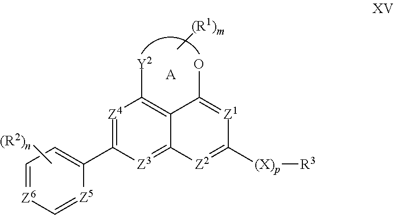

wherein R¹, R², R³, R⁹, A, X, Y², Z¹, Z², Z³, Z⁴, m, n, and p are as described herein; Z⁵ is N or C(R¹⁵); and Z⁶ is N or C(R¹⁶)

In some embodiments, the disclosure provides a compound of the formula XVI, or a pharmaceutically acceptable salt thereof,

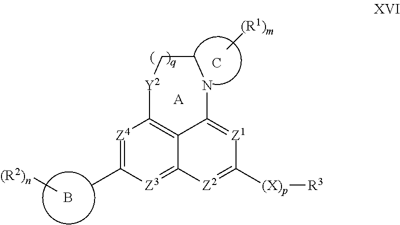

wherein R¹, R², R³, A, B, X, Y², Z¹, Z², Z³, Z⁴, Z⁵, Z⁶, m, n, and p are as described herein, and ring C is a 4- to 8-membered heterocycloalkyl, and q is 0, 1, or 2.

In some embodiments, the disclosure provides a compound of the formula XVII, or a pharmaceutically acceptable salt thereof,

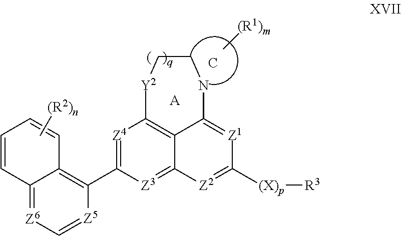

wherein R¹, R², R³, A, X, Y², Z¹, Z², Z³, Z⁴, Z⁵, Z⁶, m, n, and p are as described herein, and ring C is a 4- to 8-membered heterocycloalkyl, and q is 0, 1, or 2.

In some embodiments, the disclosure provides a compound of the formula XVII, or a pharmaceutically acceptable salt thereof,

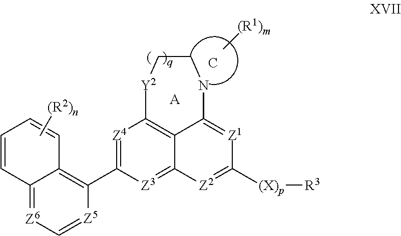

wherein R¹, R², R³, A, X, Y², Z¹, Z², Z³, Z⁴, m, n, and p are as described herein; ring C is a 4- to 8-membered heterocycloalkyl; q is 0, 1, or 2; Z⁵ is N or C(R¹⁵); and Z⁶ is N or C(R¹⁶)

In some embodiments, the disclosure provides a compound of the formula XVIII, or a pharmaceutically acceptable salt thereof,

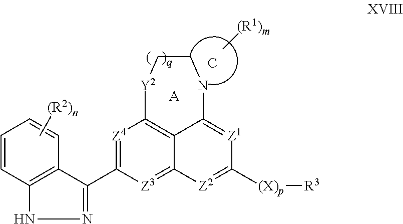

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XIX, or a pharmaceutically acceptable salt thereof,

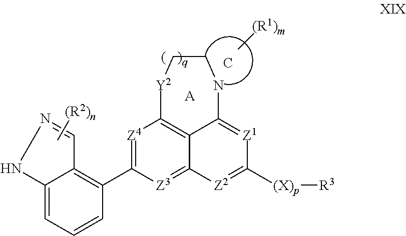

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XX, or a pharmaceutically acceptable salt thereof,

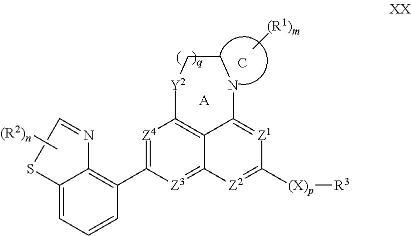

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XXI, or a pharmaceutically acceptable salt thereof,

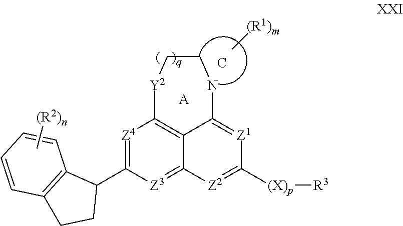

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XXII, or a pharmaceutically acceptable salt thereof,

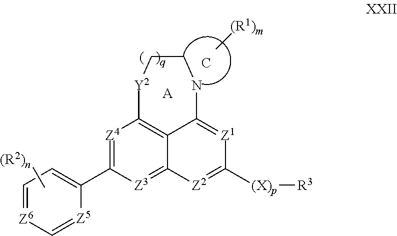

wherein R¹, R², R³, R⁹, A, X, Y², Z¹, Z², Z³, Z⁴, Z⁵, Z⁶, m, n, and p are as described herein, and ring C is a 4- to 8-membered heterocycloalkyl, and q is 0, 1, or 2.

In some embodiments, the disclosure provides a compound of the formula XXII, or a pharmaceutically acceptable salt thereof,

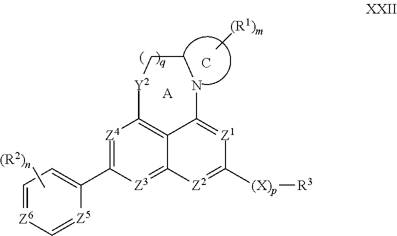

wherein R¹, R², R³, A, X, Y², Z¹, Z², Z³, Z⁴, m, n, and p are as described herein; ring C is a 4- to 8-membered heterocycloalkyl; q is 0, 1, or 2; Z⁵ is N or C(R¹⁵); and Z⁶ is N or C(R¹⁶)

In some embodiments, the disclosure provides a compound of the formula XXIII, or a pharmaceutically acceptable salt thereof,

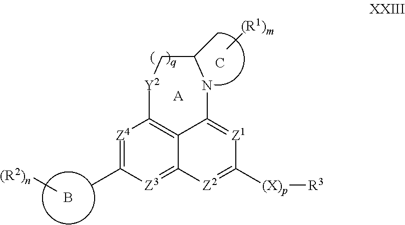

wherein R¹, R², R³, A, B, X, Y², Z¹, Z², Z³, Z⁴, Z⁵, Z⁶, m, n, and p are as described herein, and ring C is a 4- to 8-membered heterocycloalkyl, and q is 0, 1, or 2.

In some embodiments, the disclosure provides a compound of the formula XXIV, or a pharmaceutically acceptable salt thereof,

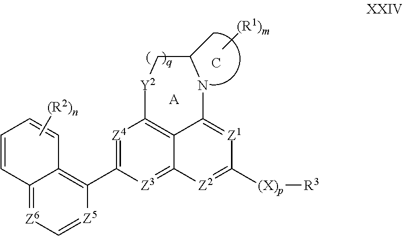

wherein R¹, R², R³, A, X, Y², Z¹, Z², Z³, Z⁴, Z⁵, Z⁶, m, n, and p are as described herein, and ring C is a 4- to 8-membered heterocycloalkyl, and q is 0, 1, or 2.

In some embodiments, the disclosure provides a compound of the formula XXIV, or a pharmaceutically acceptable salt thereof,

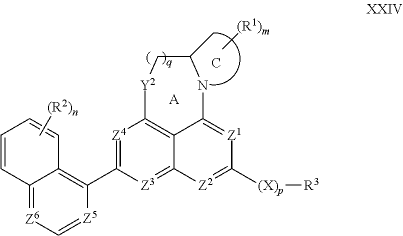

wherein R¹, R², R³, A, X, Y², Z¹, Z², Z³, Z⁴, m, n, and p are as described herein; ring C is a 4- to 8-membered heterocycloalkyl; q is 0, 1, or 2; Z⁵ is N or C(R¹⁵); and Z⁶ is N or C(R¹⁶)

In some embodiments, the disclosure provides a compound of the formula XXV, or a pharmaceutically acceptable salt thereof,

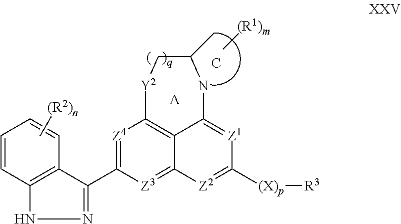

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XXVI, or a pharmaceutically acceptable salt thereof,

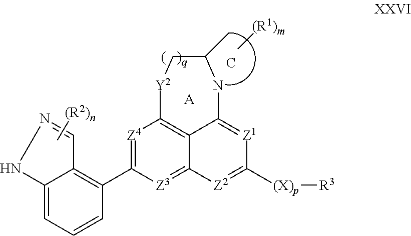

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XXVII, or a pharmaceutically acceptable salt thereof,

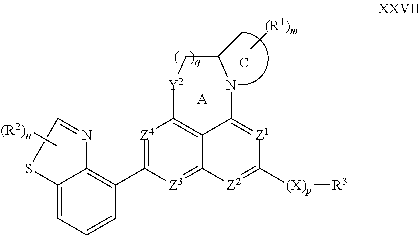

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XXVIII, or a pharmaceutically acceptable salt thereof,

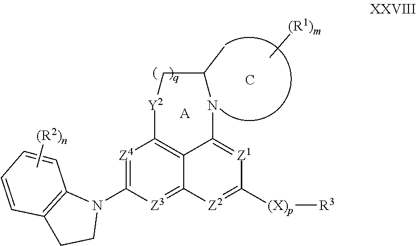

wherein R¹, R², R³, A, X, Y², Z¹, Z², Z³, Z⁴, m, n, and p are as described herein, and ring C is a 4- to 8-membered heterocycloalkyl, and q is 0, 1, or 2.

In some embodiments, the disclosure provides a compound of the formula XXIX, or a pharmaceutically acceptable salt thereof,

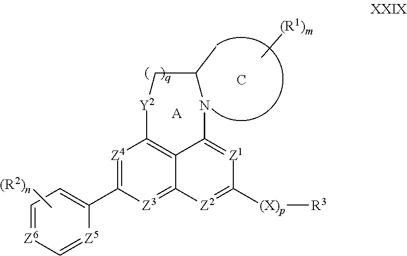

wherein R¹, R², R³, A, X, Y², Z¹, Z², Z³, Z⁴, Z⁵, Z⁶, m, n, and p are as described herein, and ring C is a 4- to 8-membered heterocycloalkyl, and q is 0, 1, or 2.

In some embodiments, the disclosure provides a compound of the formula XXIX, or a pharmaceutically acceptable salt thereof,

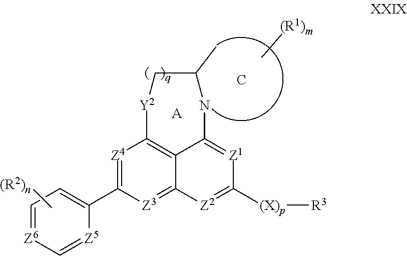

wherein R¹, R², R³, A, X, Y², Z¹, Z², Z³, Z⁴, m, n, and p are as described herein; ring C is a 4- to 8-membered heterocycloalkyl; q is 0, 1, or 2; Z⁵ is N or C(R¹⁵); and Z⁶ is N or C(R¹⁶)

In some embodiments, the disclosure provides a compound of the formula XXX, or a pharmaceutically acceptable salt thereof,

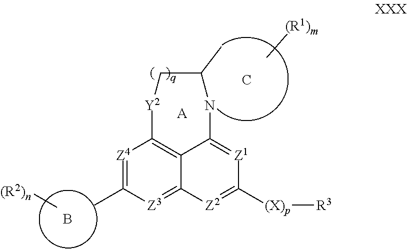

wherein R¹, R², R³, A, B, X, Y², Z¹, Z², Z³, Z⁴, Z⁵, Z⁶, m, n, and p are as described herein, and X¹ is —O—, —NH—, or —CH₂—, q is 0, 1, or 2, t is 1 or 2, and v is 1 or 2.

In some embodiments, the disclosure provides a compound of the formula XXXI, or a pharmaceutically acceptable salt thereof,

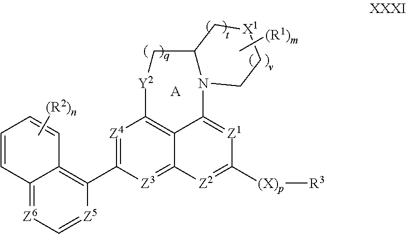

wherein R¹, R², R³, A, X, Y², Z¹, Z², Z³, Z⁴, Z⁵, Z⁶, m, n, and p are as described herein, and X¹ is —O— or —CH₂—, q is 0, 1, or 2, t is 1 or 2, and v is 1 or 2.

In some embodiments, the disclosure provides a compound of the formula XXXI, or a pharmaceutically acceptable salt thereof,

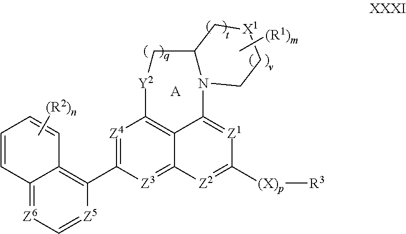

wherein R¹, R², R³, A, X, Y², Z¹, Z², Z³, Z⁴, m, n, and p are as described herein, and X¹ is —O— or —CH₂—, q is 0, 1, or 2, t is 1 or 2, v is 1 or 2, Z⁵ is N or C(R¹⁵), and Z⁶ is N or C(R¹⁶)

In some embodiments, the disclosure provides a compound of the formula XXXII, or a pharmaceutically acceptable salt thereof,

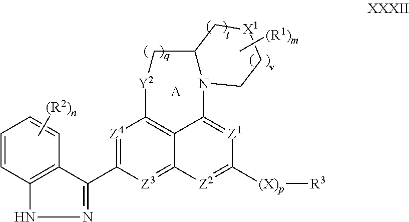

wherein R¹, R², R³, A, X, Y², Z¹, Z², Z³, Z⁴, m, n, and p are as described herein, and X¹ is —O— or —CH₂—, q is 0, 1, or 2, t is 1 or 2, and v is 1 or 2.

In some embodiments, the disclosure provides a compound of the formula XXXIII, or a pharmaceutically acceptable salt thereof,

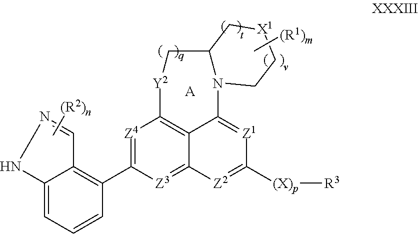

wherein R¹, R², R³, A, X, Y², Z¹, Z², Z³, Z⁴, m, n, and p are as described herein, and X¹ is —O—, —NH—, or —CH₂—, q is 0, 1, or 2, t is 1 or 2, and v is 1 or 2.

In some embodiments, the disclosure provides a compound of the formula XXXIV, or a pharmaceutically acceptable salt thereof,

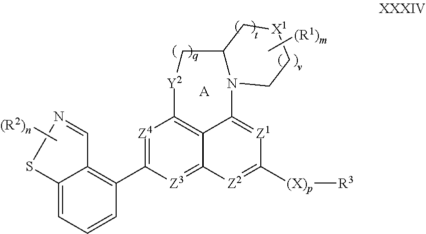

wherein R¹, R², R³, A, X, Y², Z¹, Z², Z³, Z⁴, m, n, and p are as described herein, and X¹ is —O— or —CH₂—, q is 0, 1, or 2, t is 1 or 2, and v is 1 or 2.

In some embodiments, the disclosure provides a compound of the formula XXXV, or a pharmaceutically acceptable salt thereof,

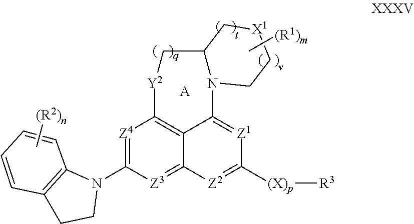

wherein R¹, R², R³, A, X, Y², Z¹, Z², Z³, Z⁴, m, n, and p are as described herein, and X¹ is —O— or —CH₂—, q is 0, 1, or 2, t is 1 or 2, and v is 1 or 2.

In some embodiments, the disclosure provides a compound of the formula XXXVI, or a pharmaceutically acceptable salt thereof,

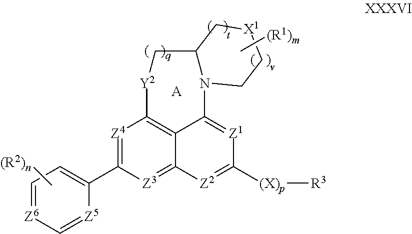

wherein R¹, R², R³, A, X, Y², Z¹, Z², Z³, Z⁴, Z⁵, Z⁶, m, n, and p are as described herein, and X¹ is —O— or —CH₂—, q is 0, 1, or 2, t is 1 or 2, and v is 1 or 2.

In some embodiments, the disclosure provides a compound of the formula XXXVI, or a pharmaceutically acceptable salt thereof,

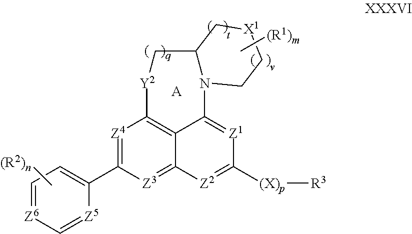

wherein R¹, R², R³, A, X, Y², Z¹, Z², Z³, Z⁴, m, n, and p are as described herein, and X¹ is —O— or —CH₂—, q is 0, 1, or 2, t is 1 or 2, v is 1 or 2, Z⁵ is N or C(R¹⁵), and Z⁶ is N or C(R¹⁶)

In some embodiments, the disclosure provides a compound of the formula XXXVII, or a pharmaceutically acceptable salt thereof,

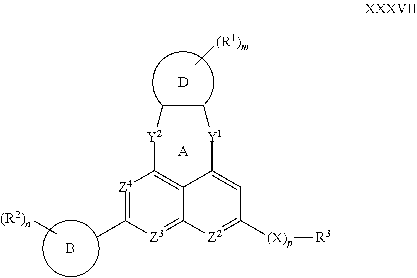

wherein R¹, R², R³, A, B, X, Y², Y², Z¹, Z², Z³, Z⁴, Z⁵, Z⁶, m, n, and p are as described herein, and ring D is a C₃-C₆ cycloalkyl or a 4- to 8-membered heterocycloalkyl.

In some embodiments, the disclosure provides a compound of the formula XXXVIII, or a pharmaceutically acceptable salt thereof,

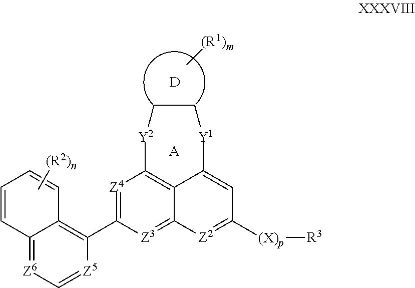

wherein R¹, R², R³, A, X, Y¹, Y², Z¹, Z², Z³, Z⁴, Z⁵, Z⁶, m, n, and p are as described herein, and ring D is a C₃-C₆ cycloalkyl or a 4- to 8-membered heterocycloalkyl.

In some embodiments, the disclosure provides a compound of the formula XXXVIII, or a pharmaceutically acceptable salt thereof,

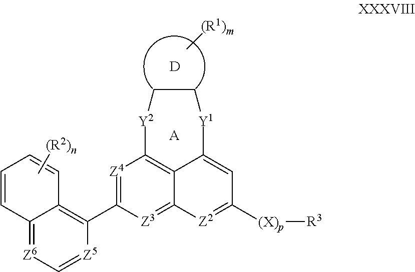

wherein R¹, R², R³, A, X, Y¹, Y², Z¹, Z², Z³, Z⁴, m, n, and p are as described herein, and ring D is a C₃-C₆ cycloalkyl or a 4- to 8-membered heterocycloalkyl, Z⁵ is N or C(R¹⁵), and Z⁶ is N or C(R¹⁶)

In some embodiments, the disclosure provides a compound of the formula XXXIX, or a pharmaceutically acceptable salt thereof,

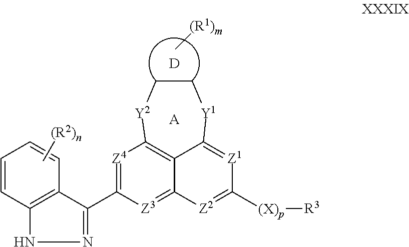

wherein R¹, R², R³, A, X, Y¹, Y², Z¹, Z², Z³, Z⁴, m, n, and p are as described herein, and ring D is a C₃-C₆ cycloalkyl or a 4- to 8-membered heterocycloalkyl.

In some embodiments, the disclosure provides a compound of the formula XXXX, or a pharmaceutically acceptable salt thereof,

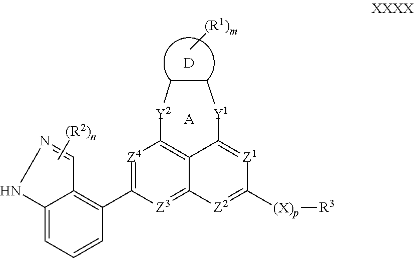

wherein R¹, R², R³, A, X, Y¹, Y², Z¹, Z², Z³, Z⁴, m, n, and p are as described herein, and ring D is a C₃-C₆ cycloalkyl or a 4- to 8-membered heterocycloalkyl.

In some embodiments, the disclosure provides a compound of the formula XXXXI, or a pharmaceutically acceptable salt thereof,

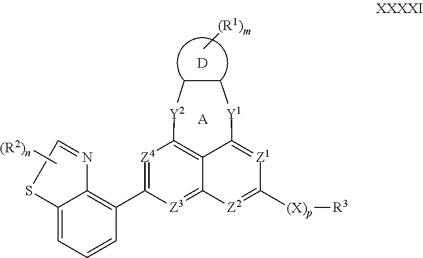

wherein R¹, R², R³, A, X, Y¹, Y², Z¹, Z², Z³, Z⁴, m, n, and p are as described herein, and ring D is a C₃-C₆ cycloalkyl or a 4- to 8-membered heterocycloalkyl.

In some embodiments, the disclosure provides a compound of the formula XXXXII, or a pharmaceutically acceptable salt thereof,

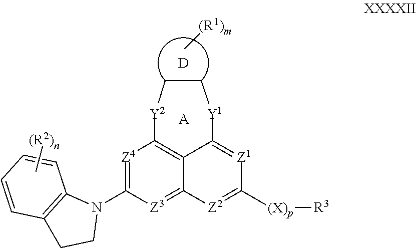

wherein R¹, R², R³, A, X, Y¹, Y², Z¹, Z², Z³, Z⁴, m, n, and p are as described herein, and ring D is a C₃-C₆ cycloalkyl or a 4- to 8-membered heterocycloalkyl.

In some embodiments, the disclosure provides a compound of the formula XXXXIII, or a pharmaceutically acceptable salt thereof,

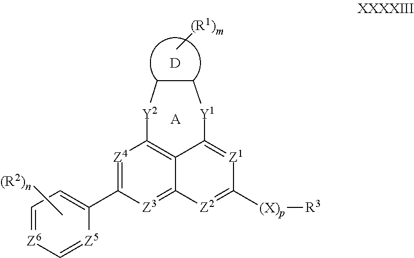

wherein R¹, R², R³, R⁹, A, X, Y¹, Y², Z¹, Z², Z³, Z⁴, Z⁵, Z⁶, m, n, and p are as described herein, and ring D is a C₃-C₆ cycloalkyl or a 4- to 8-membered heterocycloalkyl.

In some embodiments, the disclosure provides a compound of the formula XXXXIII, or a pharmaceutically acceptable salt thereof,

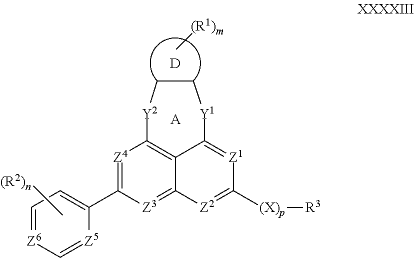

wherein R¹, R², R³, R⁹, A, X, Y¹, Y², Z¹, Z², Z³, Z⁴, m, n, and p are as described herein, and ring D is a C₃-C₆ cycloalkyl or a 4- to 8-membered heterocycloalkyl, Z⁵ is N or C(R¹⁵), and Z⁶ is N or C(R¹⁶).

In some embodiments, the disclosure provides a compound of the formula XXXXIV, or a pharmaceutically acceptable salt thereof,

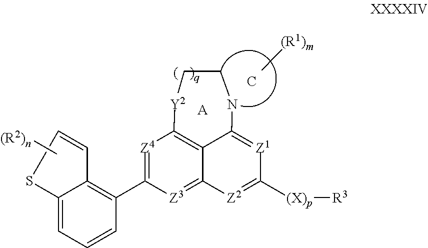

wherein R¹, R², R³, A, X, Y², Z¹, Z², Z³, Z⁴, m, n, and p are as described herein, and wherein ring C is a 4- to 8-membered heterocycloalkyl; and q is 0, 1, or 2.

In some embodiments, the disclosure provides a compound of the formula XXXXV, or a pharmaceutically acceptable salt thereof,

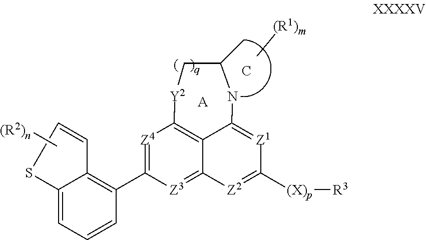

wherein R¹, R², R³, A, X, Y², Z¹, Z², Z³, Z⁴, m, n, and p are as described herein, and wherein ring C is a 4- to 8-membered heterocycloalkyl; and q is 0, 1, or 2.

In some embodiments, the disclosure provides a compound of the formula XXXXVI, or a pharmaceutically acceptable salt thereof,

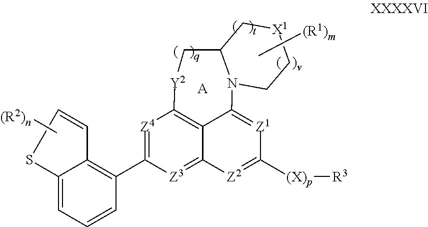

wherein R¹, R², R³, A, X, Y², Z¹, Z², Z³, Z⁴, m, n, and p are as described herein, and wherein ring C is a 4- to 8-membered heterocycloalkyl; X¹ is —O—, —NH— or —CH₂—; q is 0, 1, or 2; t is 1 or 2, and v is 1 or 2.

In some embodiments, at least one hydrogen atom in the compound of the formula I is substituted by a deuterium. In some embodiments, at least one hydrogen atom in R¹, R², or R³ in the compound of the formula I is substituted by a deuterium.

In some embodiments, Y¹ is —O—, —S—, —S(O)—, —S(O)₂—, —NR^{g}—, or —CR¹¹R¹²—. In some embodiments, Y¹ is —O—. In some embodiments, Y¹ is —S—. In some embodiments, Y¹ is —S(O)—. In some embodiments, Y¹ is —S(O)₂—. In some embodiments, Y¹ is —NR^{g}—. In some embodiments, Y² is —CR¹¹R¹²—. In some embodiments, Y² is —O—, —S—, —S(O)—, —S(O)₂—, —NR¹⁰—, or —CR¹³R¹⁴—. In some embodiments, Y² is —O—. In some embodiments, Y² is —S—. In some embodiments, Y² is —S(O)—. In some embodiments, Y² is —S(O)₂—. In some embodiments, Y¹ is —NR¹⁰—. In some embodiments, Y² is —CR¹³R¹⁴—.

In some embodiments, ring A is a C₅-C₈ cycloalkyl or 5- to 8-membered heterocycloalkyl, wherein each of C₅-C₈ cycloalkyl or 5- to 8-membered heterocycloalkyl is unsubstituted or substituted with one or more of R¹. In some embodiments, ring A is an unsubstituted C₅-C₈ cycloalkyl, or a C₅-C₈ cycloalkyl substituted with one or more of R¹. In some embodiments, ring A is an unsubstituted 5- to 8-membered heterocycloalkyl, or a 5- to 8-membered heterocycloalkyl substituted with one or more of R¹.

In some embodiments, ring A is a C₅-C₈ cycloalkyl or 5- to 8-membered heterocycloalkyl, wherein each of C₅-C₅ cycloalkyl or 5- to 8-membered heterocycloalkyl is unsubstituted or substituted with 1, 2, 3, 4, 5, or 6 of R¹. In some embodiments, ring A is an unsubstituted C₅-C₈ cycloalkyl, or a C₅-C₈ cycloalkyl substituted with 1, 2, 3, 4, 5, or 6 of R¹. In some embodiments, ring A is an unsubstituted 5- to 8-membered heterocycloalkyl, or a 5- to 8-membered heterocycloalkyl substituted with 1, 2, 3, 4, 5, or 6 of R¹.

In some embodiments, ring A is a 5- to 8-membered heterocycloalkyl, wherein the 5- to 8-membered heterocycloalkyl is unsubstituted or substituted with one or more of R¹, and/or two R¹ on ring A, taken together with the atom or atoms to which they are attached, combine to form a 4- to 8-membered heterocycloalkyl (ring C) that is unsubstituted or substituted with one or more of R¹. In some embodiments, ring A is a 5- to 8-membered heterocycloalkyl optionally substituted with one or more of R¹, and/or two R¹ on ring A, taken together with the atom or atoms to which they are attached, combine to form a 4- to 8-membered heterocycloalkyl having no more than one nitrogen atom in the 4- to 8-membered heterocycloalkyl (ring C) that is unsubstituted or substituted with one or more of R¹. In some embodiments, ring A is a 5- to 8-membered heterocycloalkyl optionally substituted with one or more of R¹, and/or two R¹ on ring A, taken together with the atom or atoms to which they are attached, combine to form a 4- to 8-membered heterocycloalkyl having no more than one nitrogen atom in the 4- to 8-membered heterocycloalkyl and having one or more —O— and/or —S— in the 4- to 8-membered heterocycloalkyl (ring C) that is unsubstituted or substituted with one or more of R¹.

In some embodiments, ring A is a 5- to 7-membered heterocycloalkyl, wherein the 5- to 7-membered heterocycloalkyl is unsubstituted or substituted with one or more of R¹, and/or two R¹ on ring A, taken together with the atom or atoms to which they are attached, combine to form a 4- to 8-membered heterocycloalkyl (ring C) that is unsubstituted or substituted with one or more of R¹. In some embodiments, ring A is a 5- to 7-membered heterocycloalkyl optionally substituted with one or more of R¹, and/or two R¹ on ring A, taken together with the atom or atoms to which they are attached, combine to form a 4- to 8-membered heterocycloalkyl having no more than one nitrogen atom in the 4- to 8-membered heterocycloalkyl (ring C) that is unsubstituted or substituted with one or more of R¹. In some embodiments, ring A is a 5- to 7-membered heterocycloalkyl optionally substituted with one or more of R¹, and/or two R¹ on ring A, taken together with the atom or atoms to which they are attached, combine to form a 4- to 8-membered heterocycloalkyl having no more than one nitrogen atom in the 4- to 8-membered heterocycloalkyl and having one or more —O— and/or —S— in the 4- to 8-membered heterocycloalkyl (ring C) that is unsubstituted or substituted with one or more of R¹.

In some embodiments, ring A is a 5- to 8-membered heterocycloalkyl, wherein 5- to 8-membered heterocycloalkyl is unsubstituted or substituted with one or more of R¹, and/or two R¹ on ring A, taken together with the atom or atoms to which they are attached, combine to form a C₄-C₅ cycloalkyl or 4- to 8-membered heterocycloalkyl (ring D) that is unsubstituted or substituted with one or more of R¹. In some embodiments, ring A is a 5- to 8-membered heterocycloalkyl optionally substituted with one or more of R¹, and/or two R¹ on ring A, taken together with the atom or atoms to which they are attached, combine to form a C₄-C₈ cycloalkyl (ring D) that is unsubstituted or substituted with one or more of R¹. In some embodiments, ring A is a 5- to 8-membered heterocycloalkyl optionally substituted with one or more of R¹, and/or two R¹ on ring A, taken together with the atom or atoms to which they are attached, combine to form a C₄-C₈ cycloalkyl (ring D) that is unsubstituted or substituted with one or more of R¹. In some embodiments, ring A is a 5- to 8-membered heterocycloalkyl optionally substituted with one or more of R¹, and/or two R¹ on ring A, taken together with the atom or atoms to which they are attached, combine to form a 4- to 8-membered heterocycloalkyl (ring D) that is unsubstituted or substituted with one or more of R¹. In some embodiments, ring A is a 5- to 8-membered heterocycloalkyl optionally substituted with one or more of R¹, and/or two R¹ on ring A, taken together with the atom or atoms to which they are attached, combine to form a 4- to 8-membered heterocycloalkyl (ring D) that is unsubstituted or substituted with one or more of R¹.

In some embodiments, ring A is a 5- to 8-membered heterocycloalkyl, wherein the 5- to 8-membered heterocycloalkyl is unsubstituted or substituted with 1, 2, 3, 4, 5, or 6 of R¹; and/or two R¹ on ring A, taken together with the atom or atoms to which they are attached, combine to form a 4- to 8-membered heterocycloalkyl (ring C) that is unsubstituted or substituted with 1, 2, 3, of R¹. In some embodiments, ring A is a 5- to 8-membered heterocycloalkyl substituted with 1, 2, 3, 4, 5, or 6 of R¹; and/or two R¹ on ring A, taken together with the atom or atoms to which they are attached, combine to form a 4- to 8-membered heterocycloalkyl having no more than one nitrogen atom in the 4- to 8-membered heterocycloalkyl (ring C) that is unsubstituted or substituted with 1, 2, 3, of R¹. In some embodiments, ring A is a 5- to 8-membered heterocycloalkyl substituted with 1, 2, 3, 4, 5, or 6 of R¹; and/or two R¹ on ring A, taken together with the atom or atoms to which they are attached, combine to form a 4- to 8-membered heterocycloalkyl having no more than one nitrogen atom in the 4- to 8-membered heterocycloalkyl and having one or more —O— and/or —S— in the 4- to 8-membered heterocycloalkyl (ring C) that is unsubstituted or substituted with 1, 2, 3, of R¹.

In some embodiments, ring A is a 5- to 8-membered heterocycloalkyl, wherein the 5- to 7-membered heterocycloalkyl is unsubstituted or substituted with 1, 2, 3, 4, 5, or 6 of R¹; and/or two R¹ on ring A, taken together with the atom or atoms to which they are attached, combine to form a 4- to 8-membered heterocycloalkyl (ring C) that is unsubstituted or substituted with 1, 2, 3, of R¹. In some embodiments, ring A is a 5- to 7-membered heterocycloalkyl substituted with 1, 2, 3, 4, 5, or 6 of R¹; and/or two R¹ on ring A, taken together with the atom or atoms to which they are attached, combine to form a 4- to 8-membered heterocycloalkyl having no more than one nitrogen atom in the 4- to 8-membered heterocycloalkyl (ring C) that is unsubstituted or substituted with 1, 2, 3, of R¹. In some embodiments, ring A is a 5- to 7-membered heterocycloalkyl substituted with 1, 2, 3, 4, 5, or 6 of R¹; and/or two R¹ on ring A, taken together with the atom or atoms to which they are attached, combine to form a 4- to 8-membered heterocycloalkyl having no more than one nitrogen atom in the 4- to 8-membered heterocycloalkyl and having one or more —O— and/or —S— in the 4- to 8-membered heterocycloalkyl (ring C) that is unsubstituted or substituted with 1, 2, 3, of R¹.

In some embodiments, ring A is a 5- to 8-membered heterocycloalkyl having an R^{g} attached to a nitrogen atom in the 5- to 8-membered heterocycloalkyl (ring A), wherein the 5- to 8-membered heterocycloalkyl is unsubstituted or substituted with one or more of R¹, and/or the R^{g} and an R¹ on ring A, taken together with the atoms to which they are attached, combine to form a 4- to 8-membered heterocycloalkyl (ring C) that is unsubstituted or substituted with one or more of R¹. In some embodiments, ring A is a 5- to 8-membered heterocycloalkyl having an R^{g} attached to a nitrogen atom in the 5- to 8-membered heterocycloalkyl (ring A), wherein the 5- to 8-membered heterocycloalkyl is unsubstituted or substituted with one or more of R¹, and/or the R^{g} and an R¹ on ring A, taken together with the atoms to which they are attached, combine to form a 4- to 8-membered heterocycloalkyl having no more than one nitrogen atom in the 4- to 8-membered heterocycloalkyl (ring C) that is unsubstituted or substituted with one or more of R¹. In some embodiments, ring A is a 5- to 8-membered heterocycloalkyl having an R⁹ attached to a nitrogen atom in the 5- to 8-membered heterocycloalkyl (ring A), wherein the 5- to 8-membered heterocycloalkyl is unsubstituted or substituted with one or more of R¹, and/or the R⁹ and an R¹ on ring A, taken together with the atoms to which they are attached, combine to form a 4- to 8-membered heterocycloalkyl having no more than one nitrogen atom in the 4- to 8-membered heterocycloalkyl and having one or more —O— and/or —S— in the 4- to 8-membered heterocycloalkyl (ring C) that is unsubstituted or substituted with one or more of R¹.

In some embodiments, ring A is a 5- to 7-membered heterocycloalkyl having an R⁹ attached to a nitrogen atom in the 5- to 7-membered heterocycloalkyl (ring A), wherein the 5- to 7-membered heterocycloalkyl is unsubstituted or substituted with one or more of R¹, and/or the R⁹ and an R¹ on ring A, taken together with the atoms to which they are attached, combine to form a 4- to 8-membered heterocycloalkyl (ring C) that is unsubstituted or substituted with one or more of R¹. In some embodiments, ring A is a 5- to 7-membered heterocycloalkyl having an R⁹ attached to a nitrogen atom in the 5- to 7-membered heterocycloalkyl (ring A), wherein the 5- to 7-membered heterocycloalkyl is unsubstituted or substituted with one or more of R¹, and/or the R⁹ and an R¹ on ring A, taken together with the atoms to which they are attached, combine to form a 4- to 8-membered heterocycloalkyl having no more than one nitrogen atom in the 4- to 8-membered heterocycloalkyl (ring C) that is unsubstituted or substituted with one or more of R¹. In some embodiments, ring A is a 5- to 7-membered heterocycloalkyl having an R⁹ attached to a nitrogen atom in the 5- to 7-membered heterocycloalkyl (ring A), wherein the 5- to 7-membered heterocycloalkyl is unsubstituted or substituted with one or more of R¹, and/or the R⁹ and an R¹ on ring A, taken together with the atoms to which they are attached, combine to form a 4- to 8-membered heterocycloalkyl having no more than one nitrogen atom in the 4- to 8-membered heterocycloalkyl and having one or more —O— and/or —S— in the 4- to 8-membered heterocycloalkyl (ring C) that is unsubstituted or substituted with one or more of R¹.

In some embodiments, ring A is a 5- to 8-membered heterocycloalkyl having an R⁹ attached to a nitrogen atom in the 5- to 8-membered heterocycloalkyl (ring A), wherein the 5- to 8-membered heterocycloalkyl is unsubstituted or substituted with one or more of R¹, and/or the R⁹ and an R¹ on a carbon atom adjacent to the R⁹ on ring A, taken together with the atoms to which they are attached, combine to form a fused 4- to 8-membered heterocycloalkyl (ring C) that is unsubstituted or substituted with one or more of R¹. In some embodiments, ring A is a 5- to 8-membered heterocycloalkyl having an R⁹ attached to a nitrogen atom in the 5- to 8-membered heterocycloalkyl (ring A), wherein the 5- to 8-membered heterocycloalkyl is unsubstituted or substituted with one or more of R¹, and/or the R⁹ and an R¹ on a carbon atom adjacent to the R⁹ on ring A, taken together with the atoms to which they are attached, combine to form a fused 4- to 8-membered heterocycloalkyl having no more than one nitrogen atom in the fused 4- to 8-membered heterocycloalkyl (ring C) that is unsubstituted or substituted with one or more of R¹. In some embodiments, ring A is a 5- to 8-membered heterocycloalkyl having an R⁹ attached to a nitrogen atom in the 5- to 8-membered heterocycloalkyl (ring A), wherein the 5- to 8-membered heterocycloalkyl is unsubstituted or substituted with one or more of R¹, and/or the R⁹ and an R¹ on a carbon atom adjacent to the R⁹ on ring A, taken together with the atoms to which they are attached, combine to form a fused 4- to 8-membered heterocycloalkyl having no more than one nitrogen atom in the fused 4- to 8-membered heterocycloalkyl and having one or more —O— and/or —S— in the fused 4- to 8-membered heterocycloalkyl (ring C) that is unsubstituted or substituted with one or more of R¹.

In some embodiments, ring A is a 5- to 7-membered heterocycloalkyl having an R⁹ attached to a nitrogen atom in the 5- to 7-membered heterocycloalkyl (ring A), wherein the 5- to 7-membered heterocycloalkyl is unsubstituted or substituted with one or more of R¹, and/or the R⁹ and an R¹ on a carbon atom adjacent to the R⁹ on ring A, taken together with the atoms to which they are attached, combine to form a fused 4- to 8-membered heterocycloalkyl (ring C) that is unsubstituted or substituted with one or more of R¹. In some embodiments, ring A is a 5- to 7-membered heterocycloalkyl having an R⁹ attached to a nitrogen atom in the 5- to 7-membered heterocycloalkyl (ring A), wherein the 5- to 7-membered heterocycloalkyl is unsubstituted or substituted with one or more of R¹, and/or the R⁹ and an R¹ on a carbon atom adjacent to the R⁹ on ring A, taken together with the atoms to which they are attached, combine to form a fused 4- to 8-membered heterocycloalkyl having no more than one nitrogen atom in the fused 4- to 8-membered heterocycloalkyl (ring C) that is unsubstituted or substituted with one or more of R¹. In some embodiments, ring A is a 5- to 7-membered heterocycloalkyl having an R⁹ attached to a nitrogen atom in the 5- to 7-membered heterocycloalkyl (ring A), wherein the 5- to 7-membered heterocycloalkyl is unsubstituted or substituted with one or more of R¹, and/or the R⁹ and an R¹ on a carbon atom adjacent to the R⁹ on ring A, taken together with the atoms to which they are attached, combine to form a fused 4- to 8-membered heterocycloalkyl having no more than one nitrogen atom in the fused 4- to 8-membered heterocycloalkyl and having one or more —O— and/or —S— in the fused 4- to 8-membered heterocycloalkyl (ring C) that is unsubstituted or substituted with one or more of R¹.

In some embodiments, ring A is a 5- to 8-membered heterocycloalkyl having an R⁹ attached to a nitrogen atom in the 5- to 8-membered heterocycloalkyl (ring A), wherein the 5- to 8-membered heterocycloalkyl is unsubstituted or substituted with one or more of R¹, and/or the R⁹ and an R¹ on a carbon atom not adjacent to the R⁹ on ring A, taken together with the atoms to which they are attached, combine to form a bridged 5- to 8-membered heterocycloalkyl (ring C) that is unsubstituted or substituted with one or more of R¹. In some embodiments, ring A is a 5- to 8-membered heterocycloalkyl having an R⁹ attached to a nitrogen atom in the 5- to 8-membered heterocycloalkyl (ring A), wherein the 5- to 8-membered heterocycloalkyl is unsubstituted or substituted with one or more of R¹, and/or the R⁹ and an R¹ on a carbon atom not adjacent to the R⁹ on ring A, taken together with the atoms to which they are attached, combine to form a bridged 5- to 8-membered heterocycloalkyl having no more than one nitrogen atom in the 5- to 8-membered heterocycloalkyl (ring C) that is unsubstituted or substituted with one or more of R¹. In some embodiments, ring A is a 5- to 8-membered heterocycloalkyl having an R⁹ attached to a nitrogen atom in the 5- to 8-membered heterocycloalkyl (ring A), wherein the 5- to 8-membered heterocycloalkyl is unsubstituted or substituted with one or more of R¹, and/or the R⁹ and an R¹ on a carbon atom not adjacent to the R⁹ on ring A, taken together with the atoms to which they are attached, combine to form a bridged 5- to 8-membered heterocycloalkyl having no more than one nitrogen atom in the 5- to 8-membered heterocycloalkyl and having one or more —O— and/or —S— in the 5- to 8-membered heterocycloalkyl (ring C) that is unsubstituted or substituted with one or more of R¹.

In some embodiments, ring A is a 5- to 7-membered heterocycloalkyl having an R⁹ attached to a nitrogen atom in the 5- to 7-membered heterocycloalkyl (ring A), wherein the 5- to 7-membered heterocycloalkyl is unsubstituted and R⁹ is H, deuterium, —C(O)R⁹, —C(O)NR^{g}R^{h}, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{c}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, ring A is a 5- to 7-membered heterocycloalkyl having an R^{g} attached to a nitrogen atom in the 5- to 7-membered heterocycloalkyl (ring A), wherein the 5- to 7-membered heterocycloalkyl is substituted with one or more of R¹ (e.g., one R¹ or two R¹s) selected from the group consisting of C₁-C₆ alkyl, C₂-C₆ alkenyl, C₂-C₆ alkynyl, C₃-C₆ cycloalkyl, 4- to 10-membered heterocycloalkyl, 5- to 10-membered heteroaryl, and C₆-C₁₀ aryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, 5- to 10-membered heteroaryl, and C₆-C₁₀ aryl, is independently optionally substituted by deuterium, halogen, —R^{e}, —R^{f}, —OR^{c}, or —CN, or —NO₂, and R⁹ is H, deuterium, —C(O)R^{g}, —C(O)NR^{g}R^{h}, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, ring A is a 5- to 8-membered heterocycloalkyl or a 5- to 7-membered heterocycloalkyl having an R⁹ attached to a nitrogen atom in the 5- to 8-membered heterocycloalkyl or a 5- to 7-membered heterocycloalkyl (ring A), wherein R⁹ is H, deuterium, unsubstituted C₁-C₆ alkyl, or a C₁-C₆ haloalkyl.

In some embodiments, ring A is a fused 5- to 8-membered heterocycloalkyl that, optionally together with ring C or ring D, forms a core of the compound of the formula I, the core having the formula

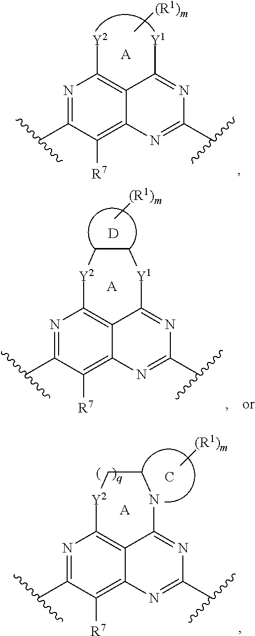

wherein each “ [symbol] ” is a point of covalent attachment to either ring B or —(X)ₚ—R³, and ring C and ring D are as defined herein.

In some embodiments, ring A is a fused 5- to 8-membered heterocycloalkyl that, optionally together with ring C or ring D, forms the core of the compound of the formula I having the formula

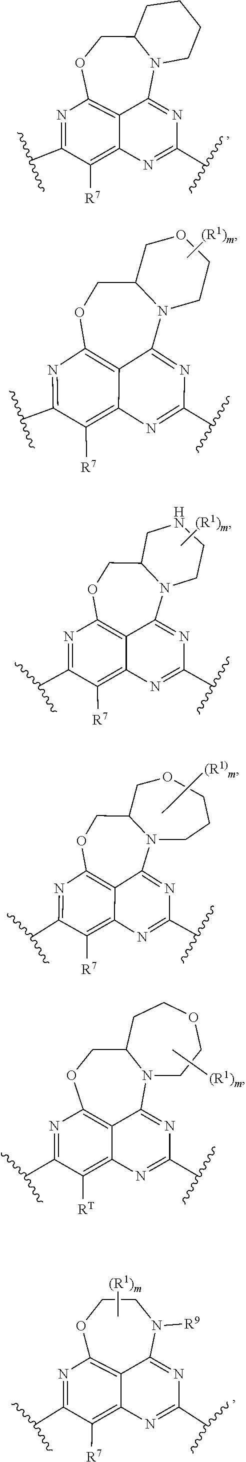

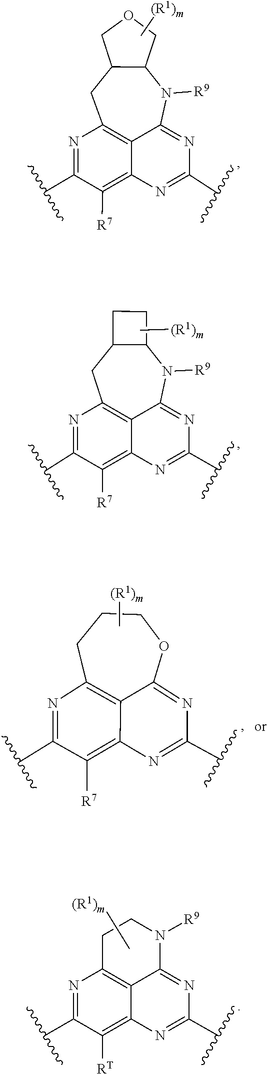

wherein each “ [symbol] ” is a point of covalent attachment, and each hydrogen atom in the piperidinyl group is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, ring A is a fused 5- to 8-membered heterocycloalkyl that, optionally together with ring C or ring D, forms the core of the compound of the formula I having the formula

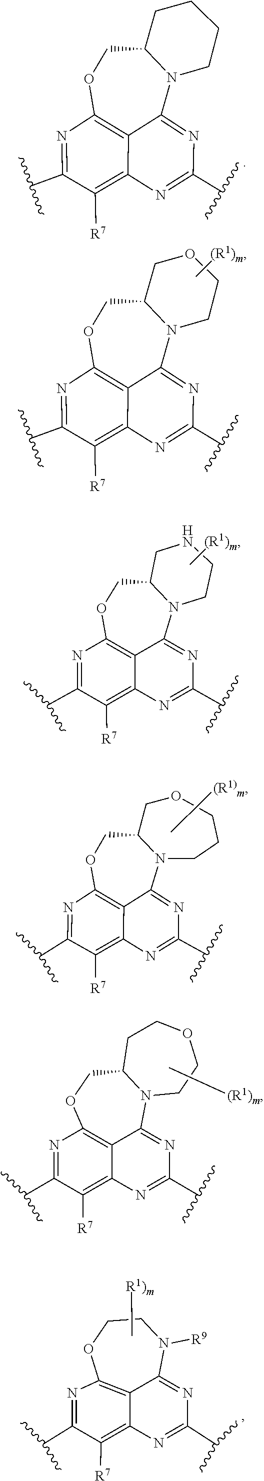

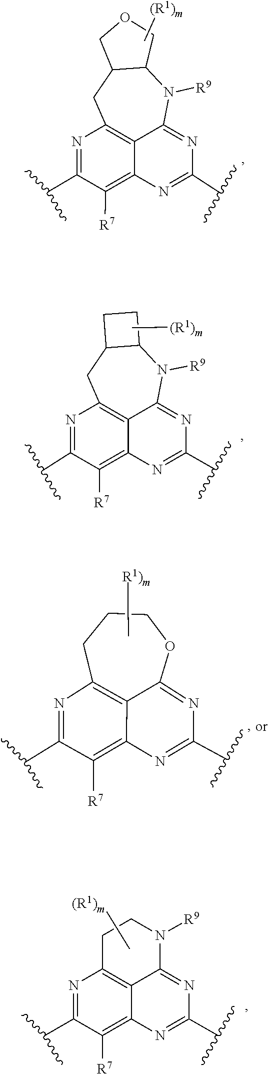

wherein each “ [symbol] ” is a point of covalent attachment, and each hydrogen atom in the piperidinyl group is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, ring A is optionally taken together with ring C or ring D to form a core of the compound of the formula I, the core having the formula

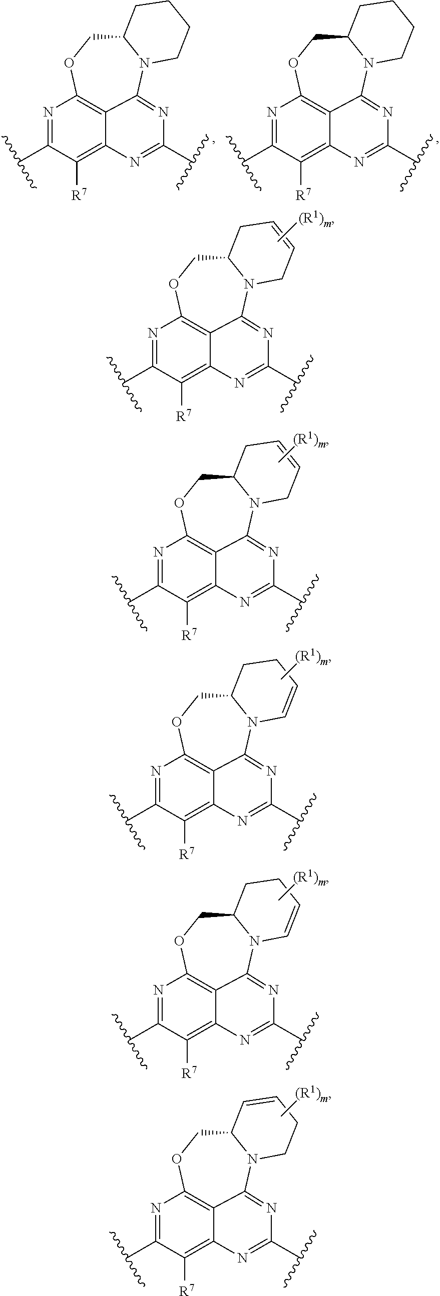

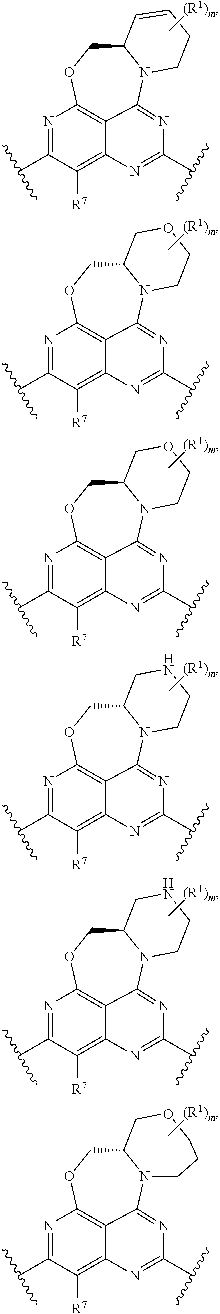

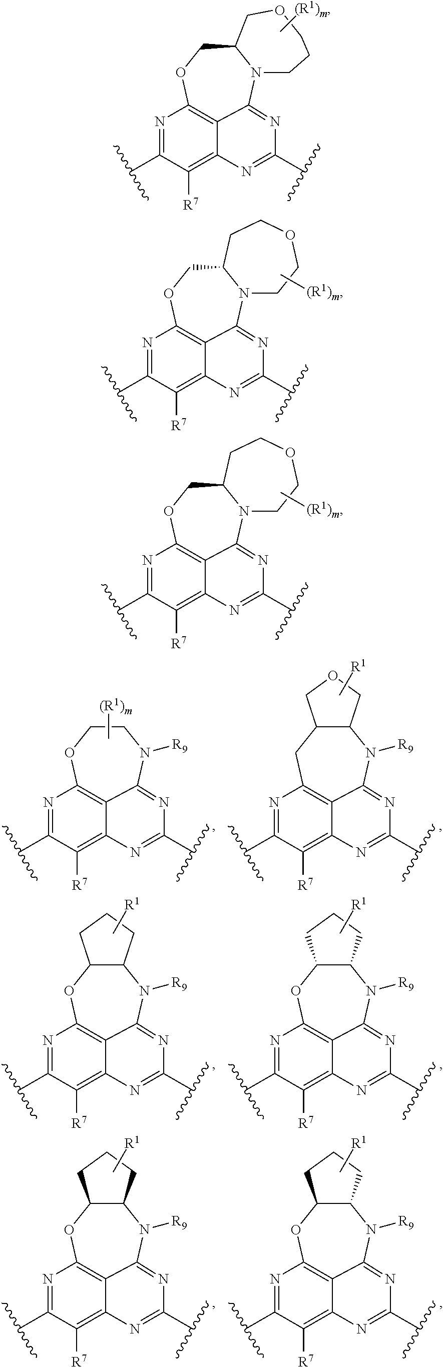

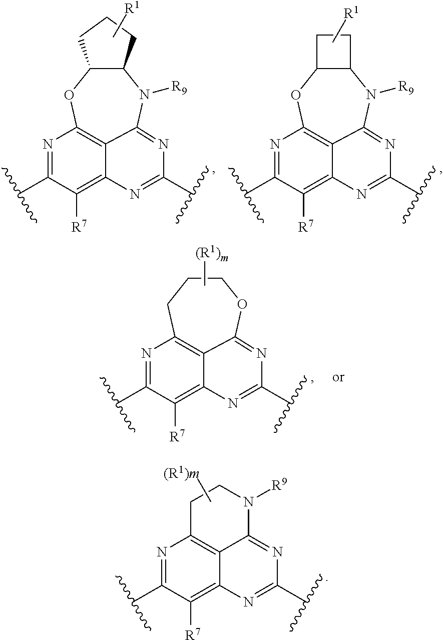

wherein each “ [symbol] ” is a point of covalent attachment to either ring B or —(X)ₚ—R³, and each hydrogen atom in ring A, ring C, if present, and ring D, if present, is independently optionally substituted with an R¹ selected from the group consisting of deuterium, halogen, C₁-C₆ alkyl, C₁-C₆ haloalkyl, —R^{c}, —R^{f}, —OR^{c}, —OC(O)R^{c}, —OC(O)NR^{c}R^{f}, —OS(O)R^{c}, —OS(O)₂R^{c}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, ring A is a fused 5- to 8-membered heterocycloalkyl that, optionally together with ring C or ring D, forms a core of the compound of the formula I, the core having the formula

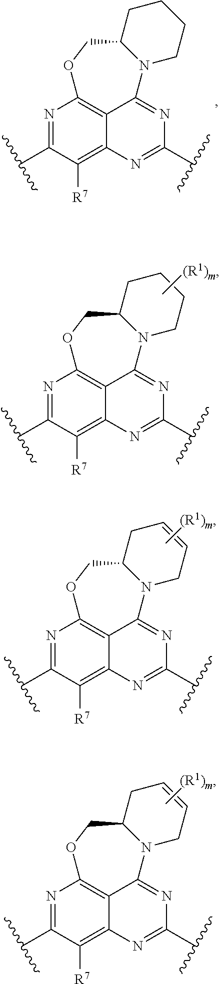

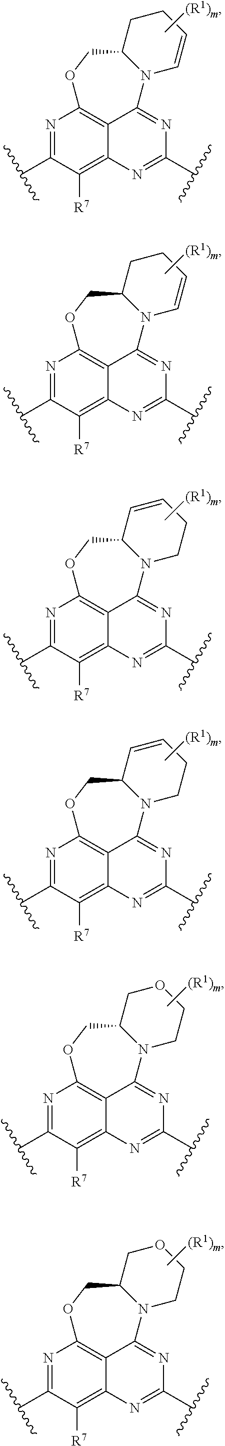

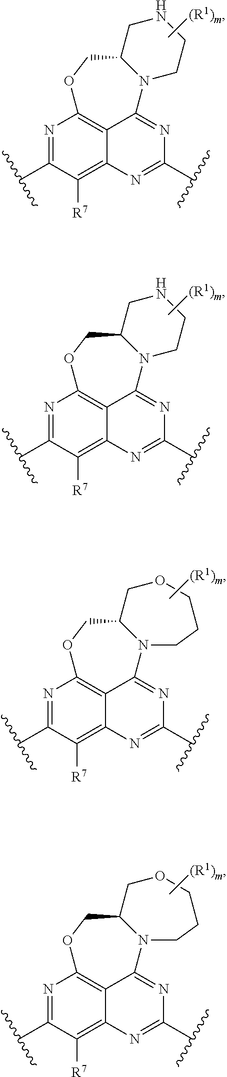

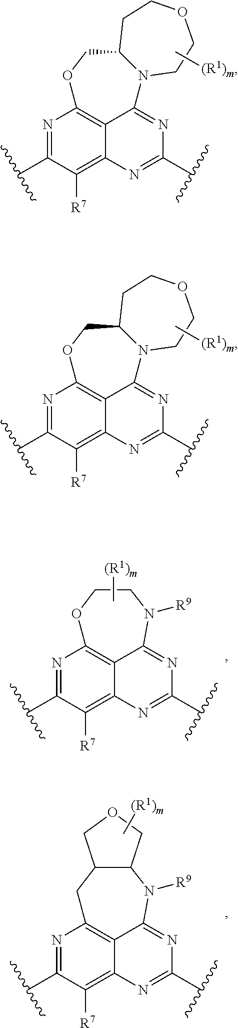

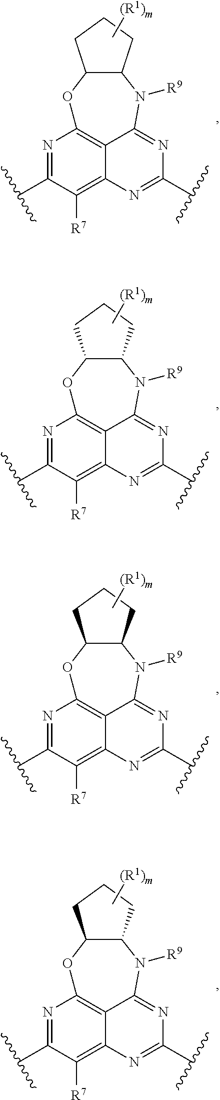

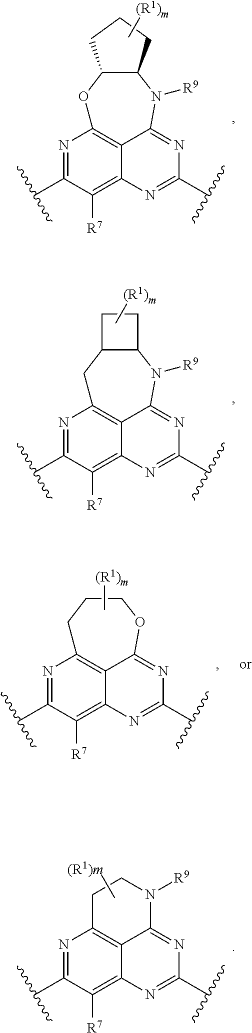

wherein each “ [symbol] ” is a point of covalent attachment to either ring B or —(X)ₚ—R³, and each hydrogen atom the piperidinyl group is independently substituted with a deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, each R¹ on ring C is independently selected from deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, each R¹ on ring D is independently selected from deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, ring A is a fused 5- to 8-membered heterocycloalkyl that, optionally together with ring C or ring D, forms the core of the compound of the formula I having the formula

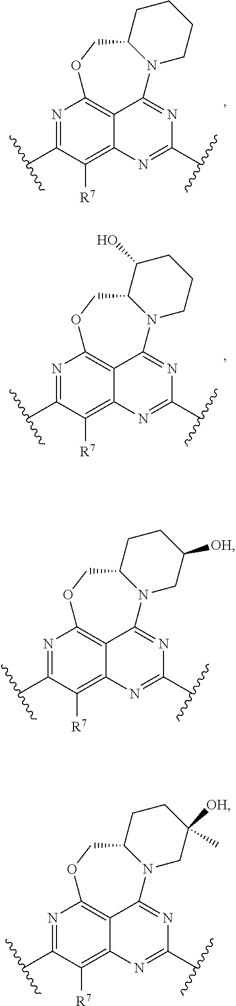

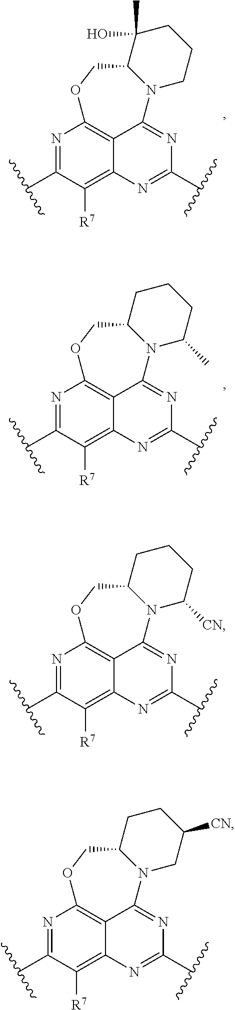

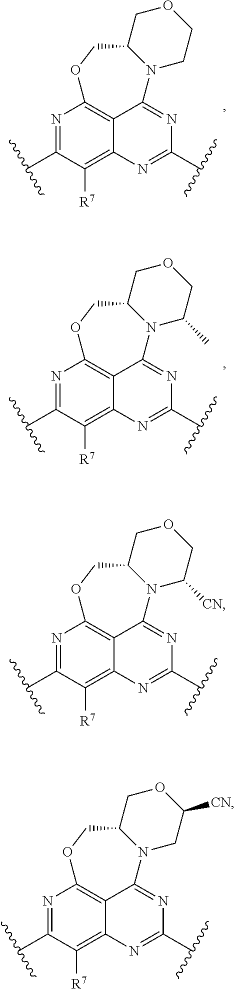

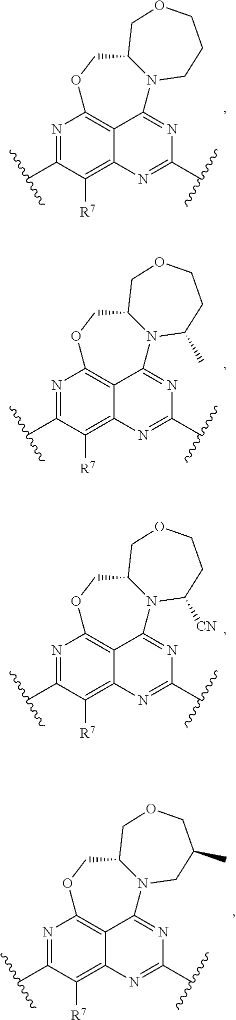

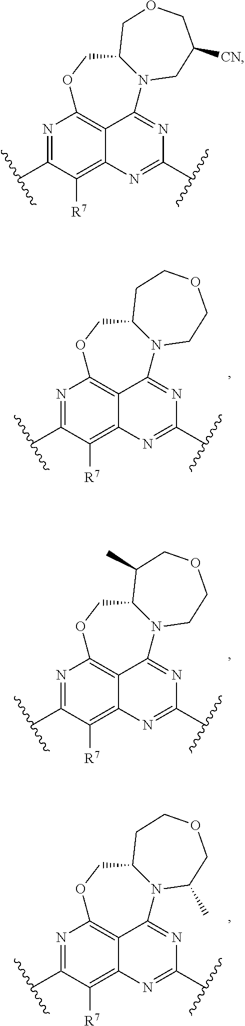

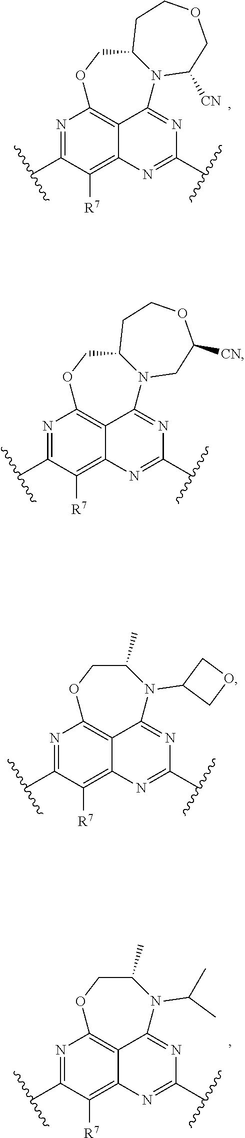

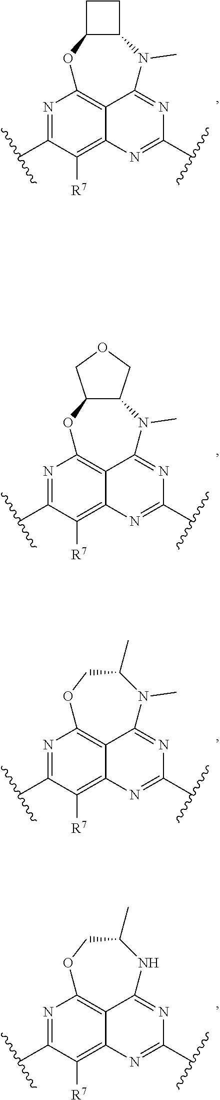

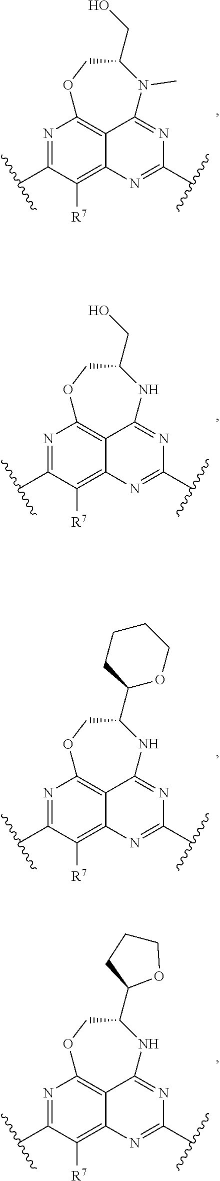

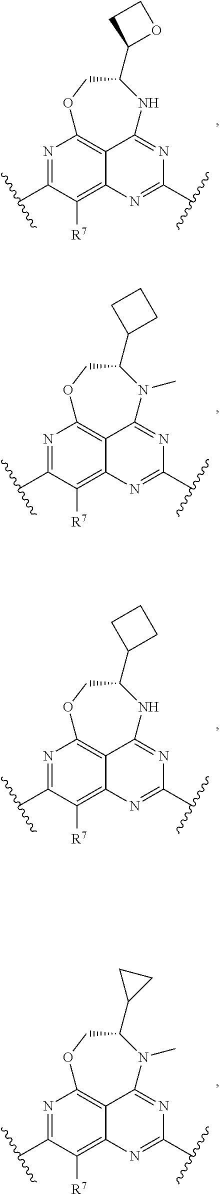

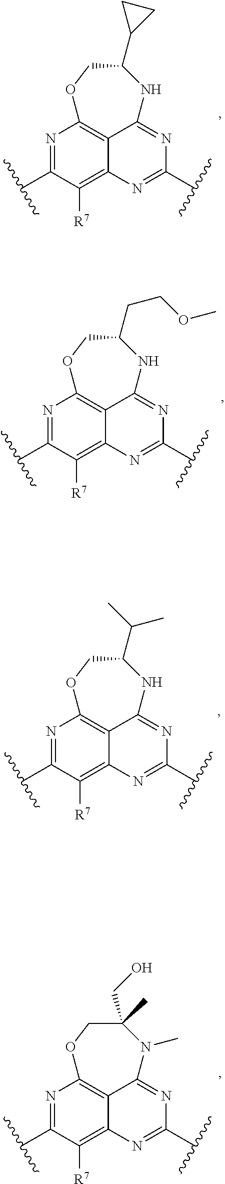

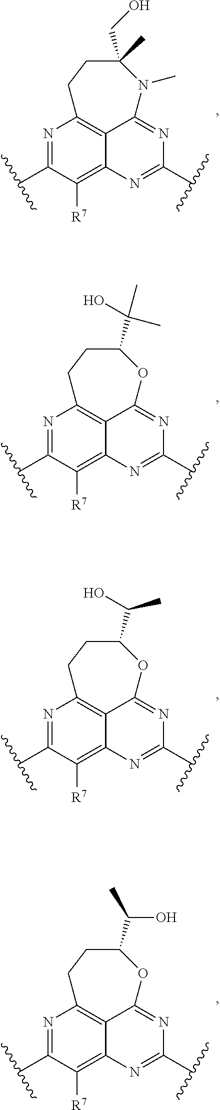

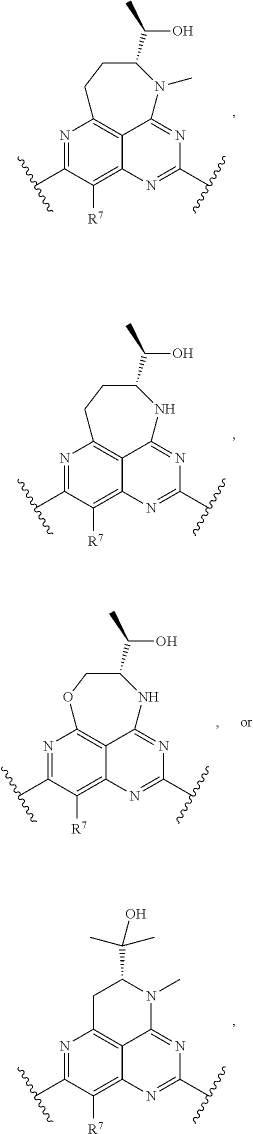

wherein each wherein “ [symbol] ” is a point of covalent attachment.

In some embodiments, ring A is a fused 5- to 8-membered heterocycloalkyl that, optionally together with ring C or ring D, forms a core of the compound of the formula I, the core having the formula

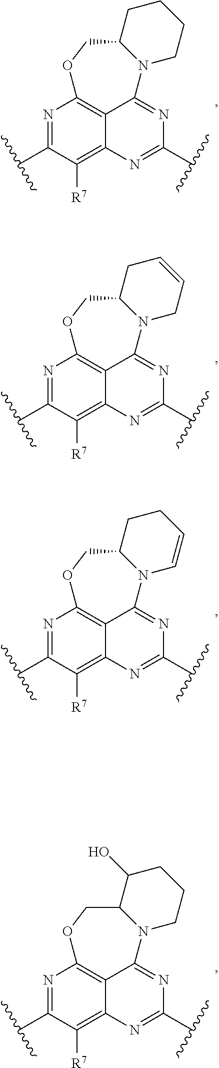

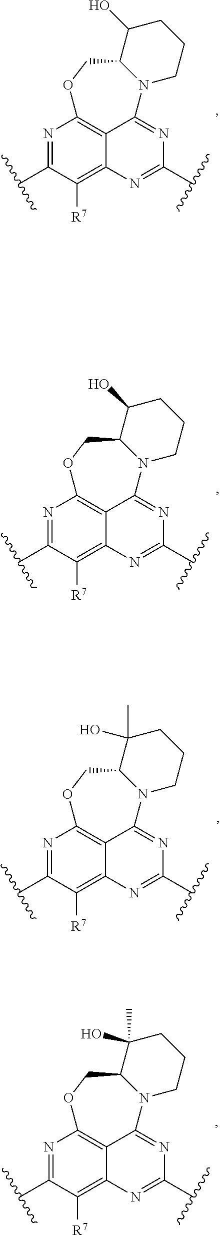

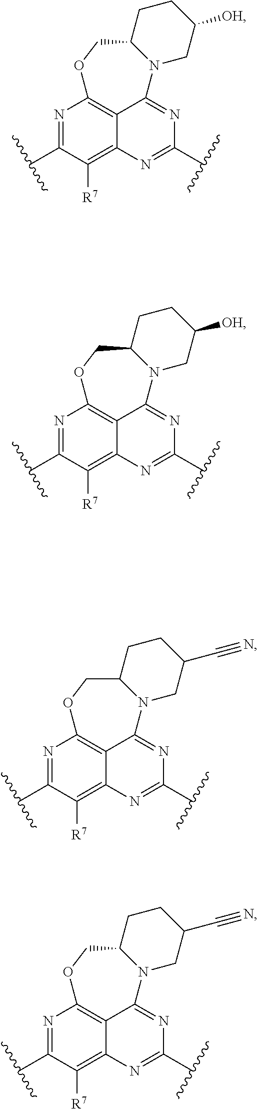

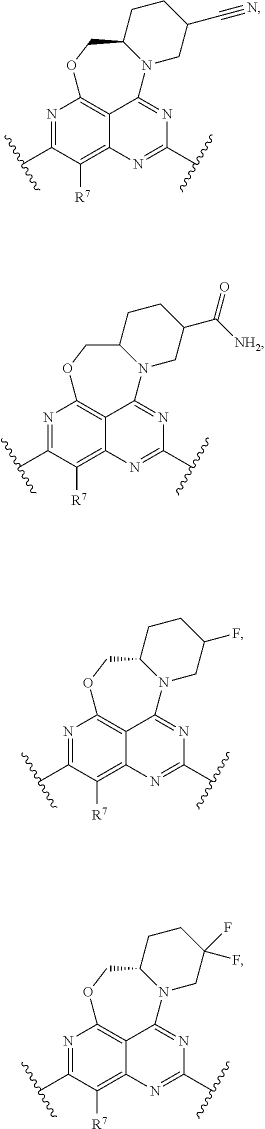

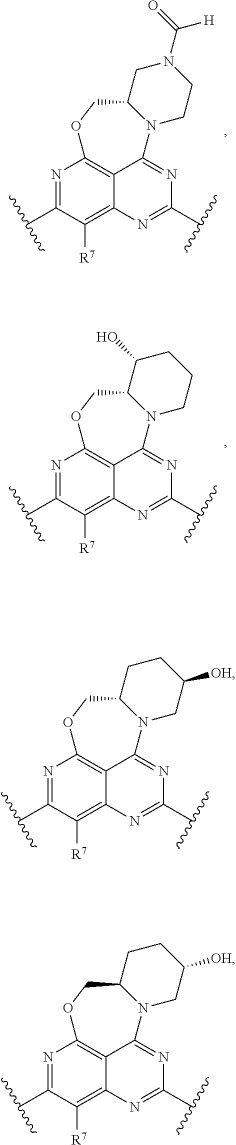

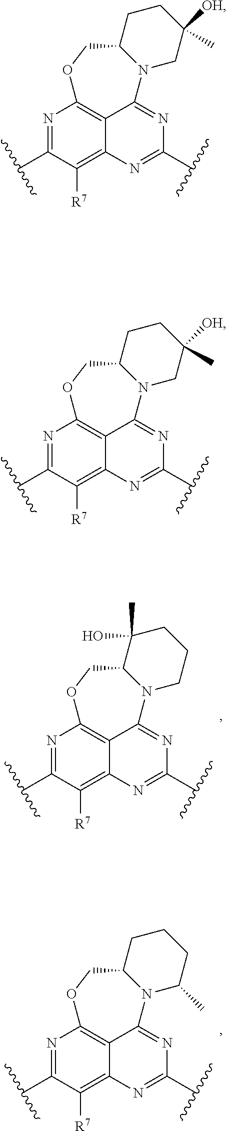

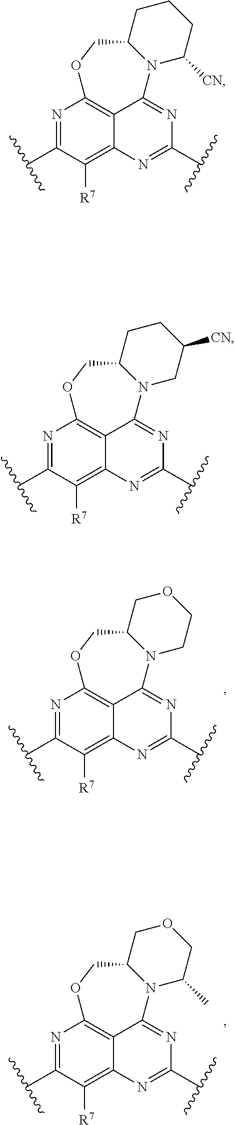

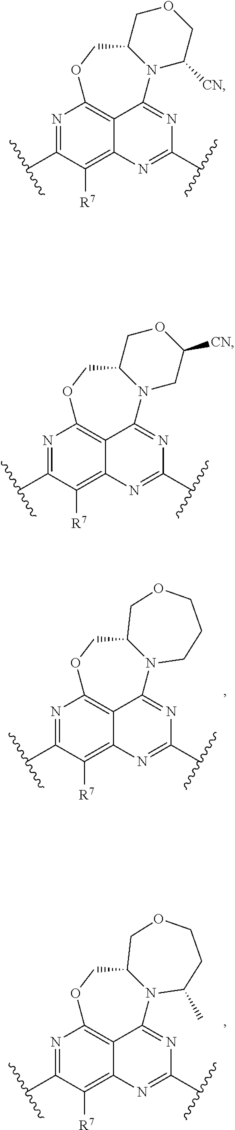

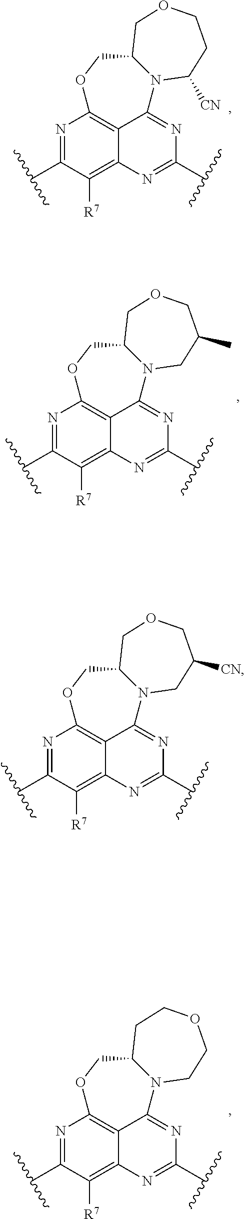

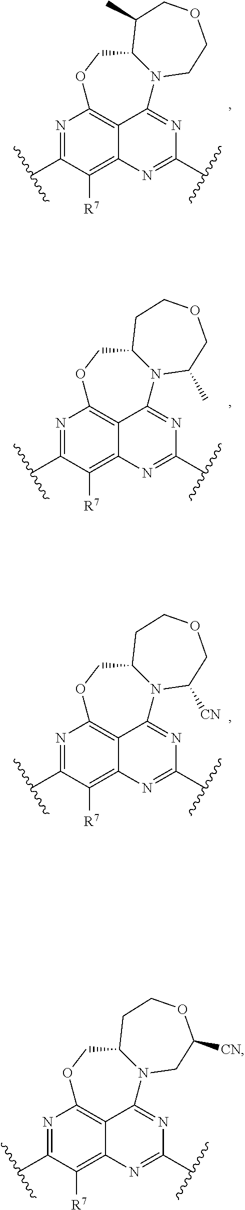

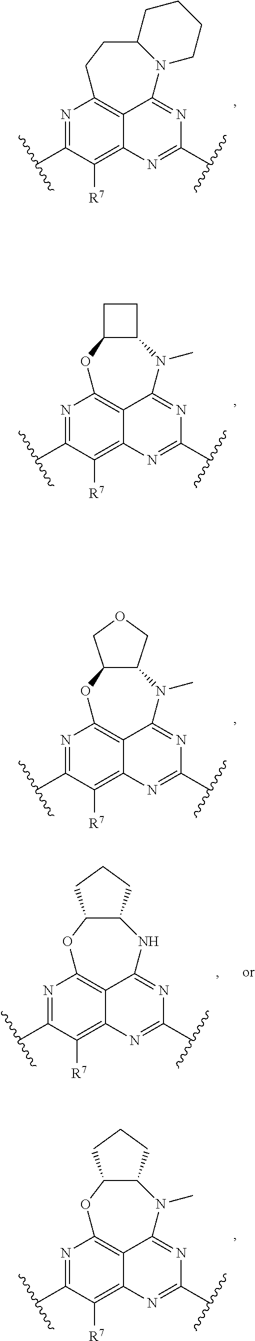

wherein each “ [symbol] ” is a point of covalent attachment to either ring B or —(X)ₚ—R³.

In some embodiments, ring A is a fused 5- to 8-membered heterocycloalkyl that forms a core of the compound of the formula I, the core having the formula

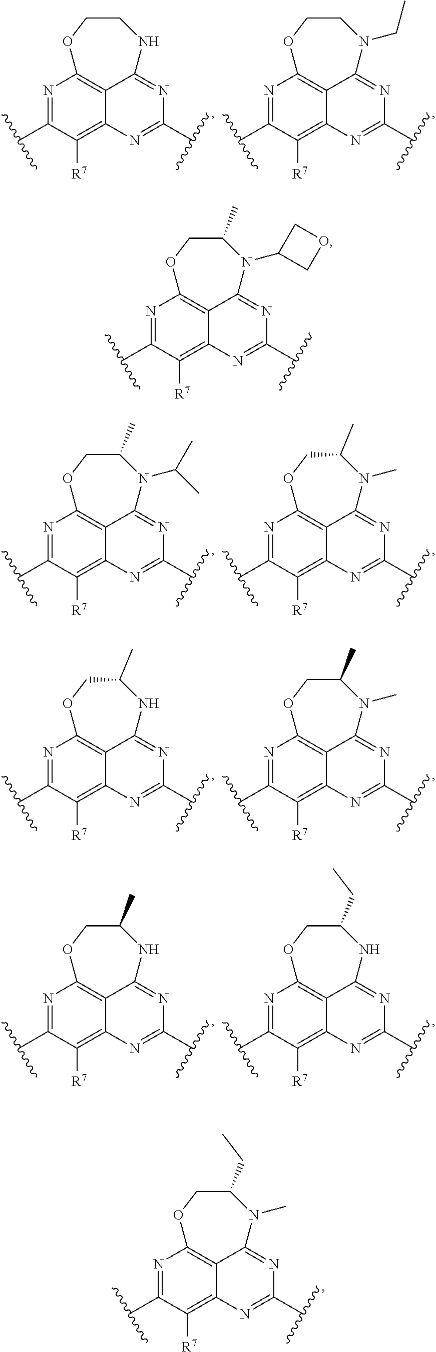

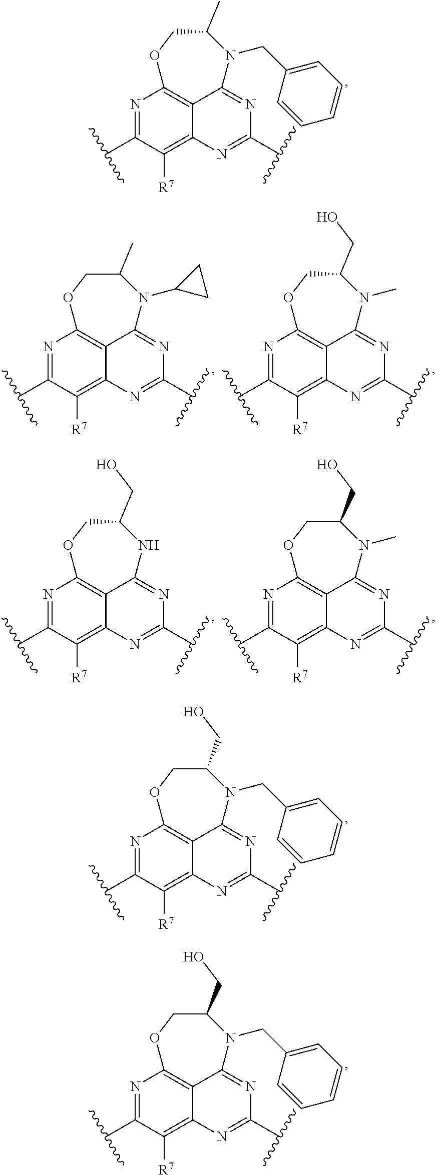

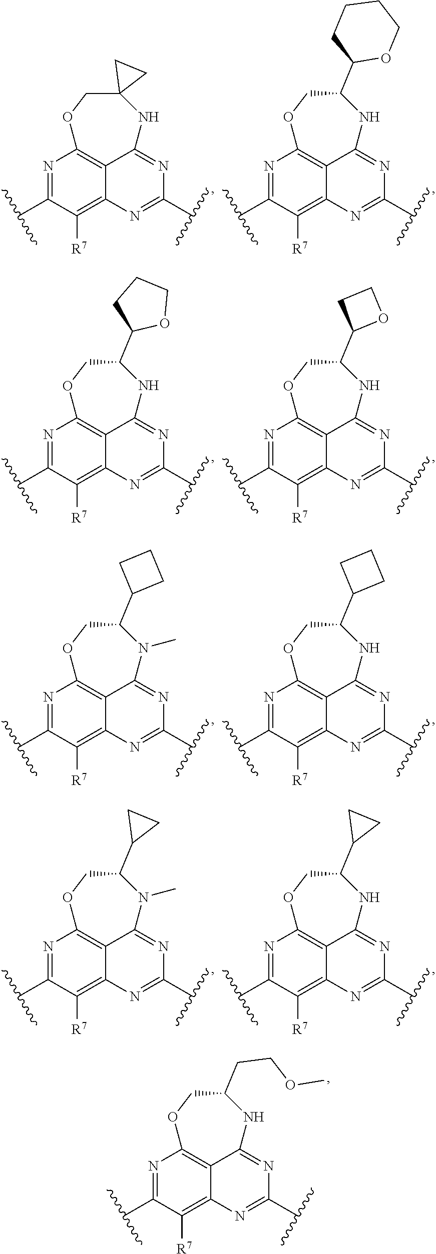

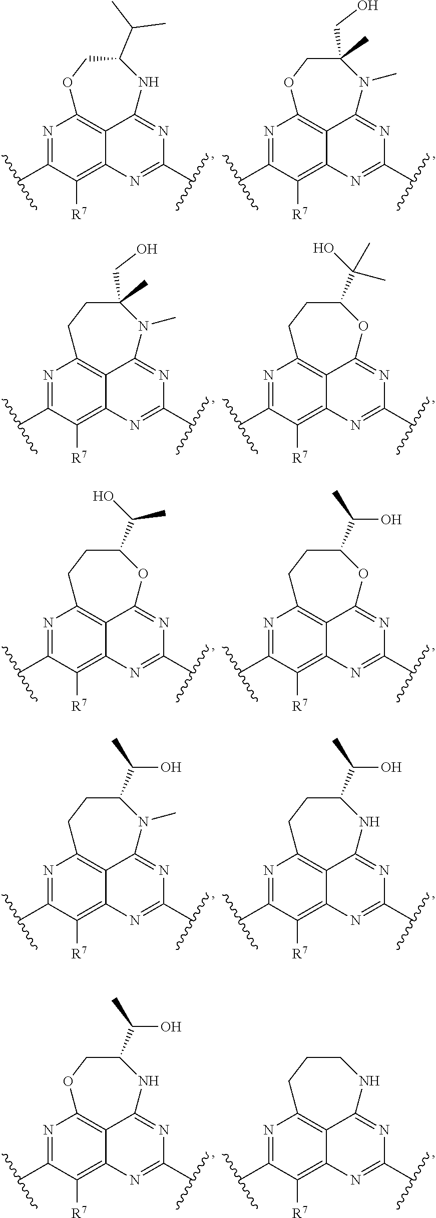

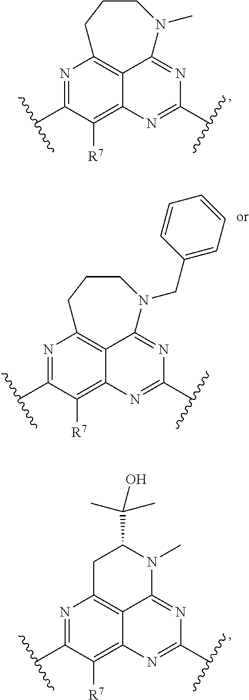

wherein each “ [symbol] ” is a point of covalent attachment to either ring B or —(X)ₚ—R³.

In some embodiments, ring A is optionally taken together with ring C or ring D to form a core of the compound of the formula 1, the core having the formula

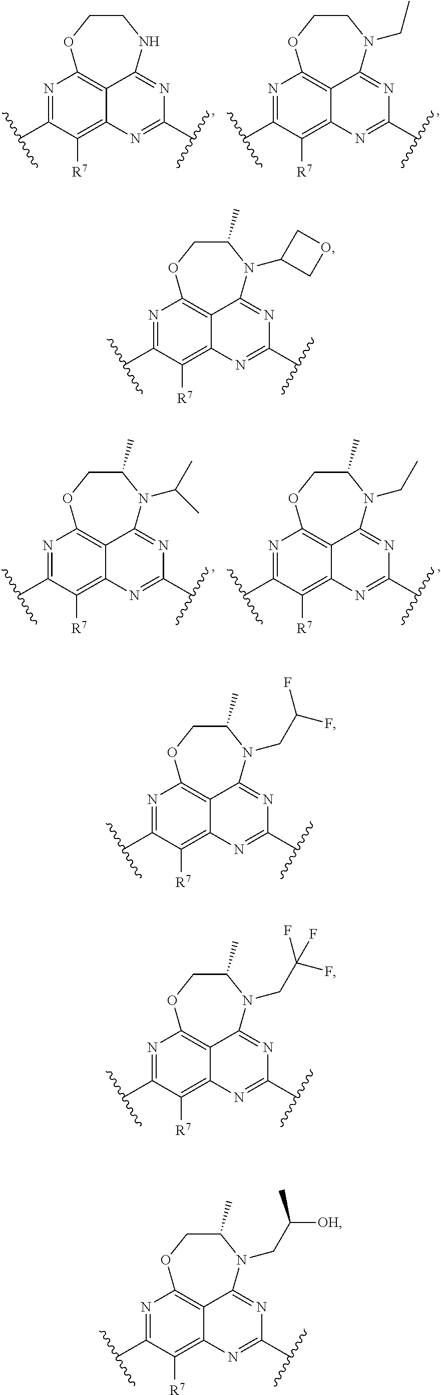

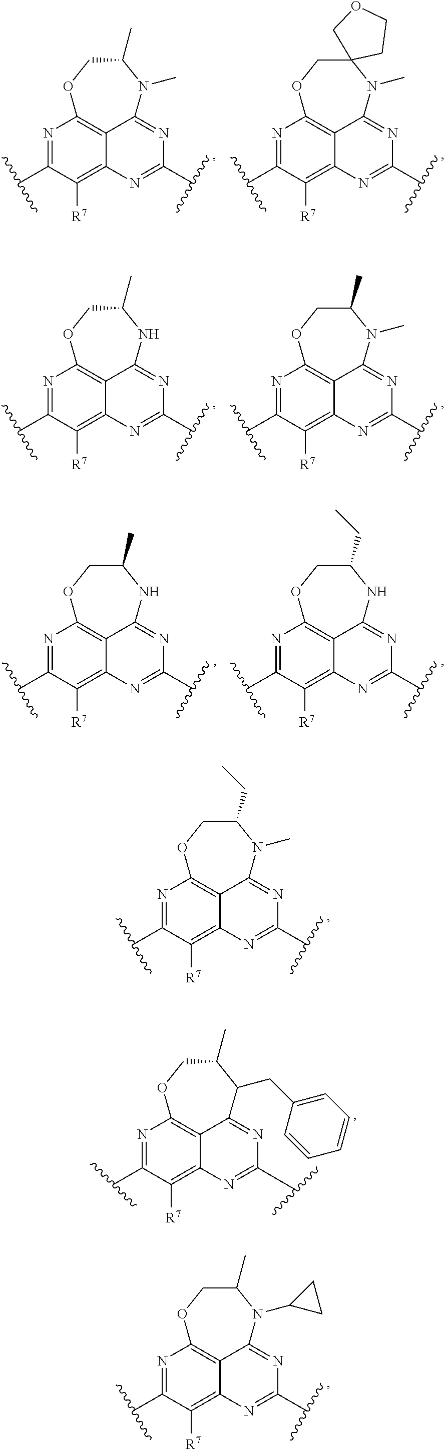

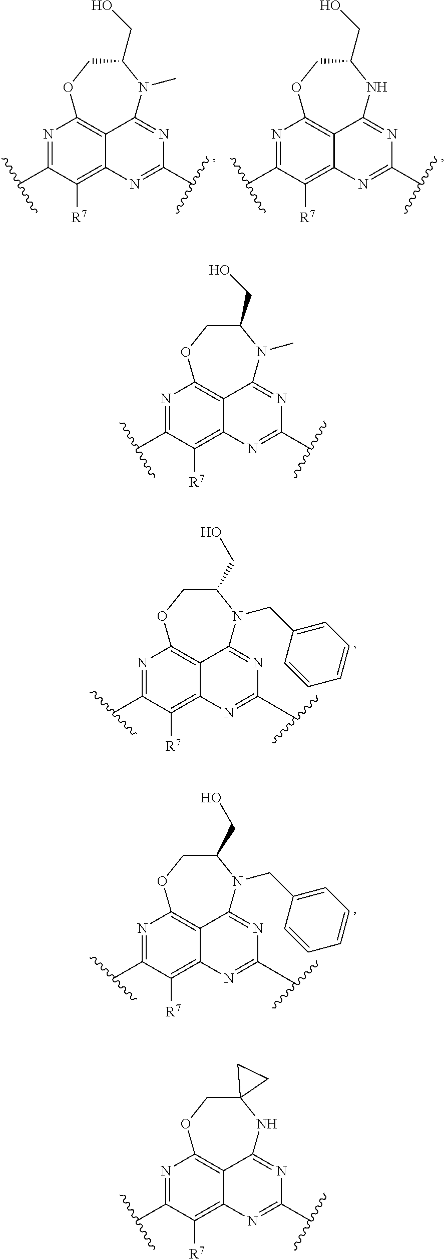

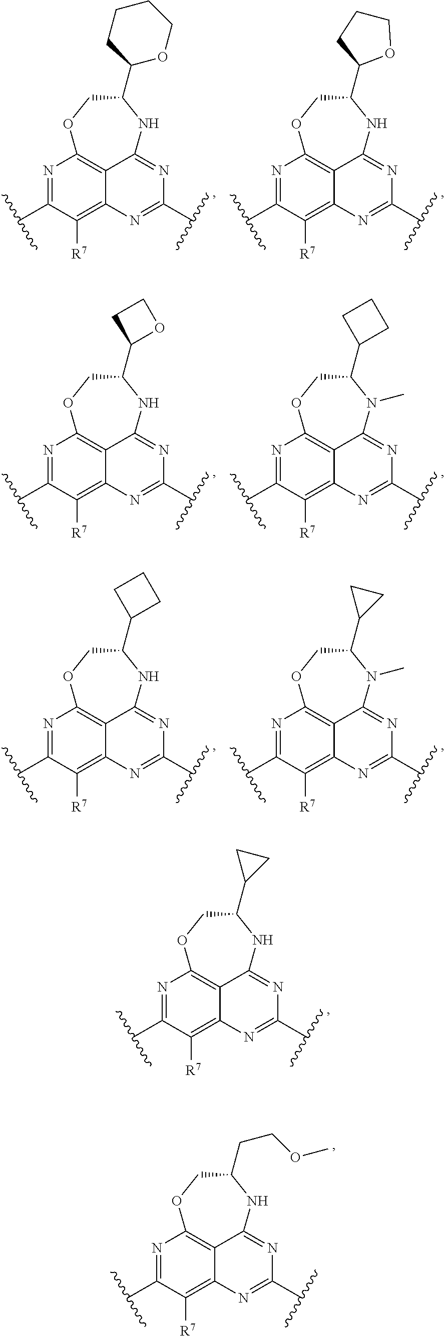

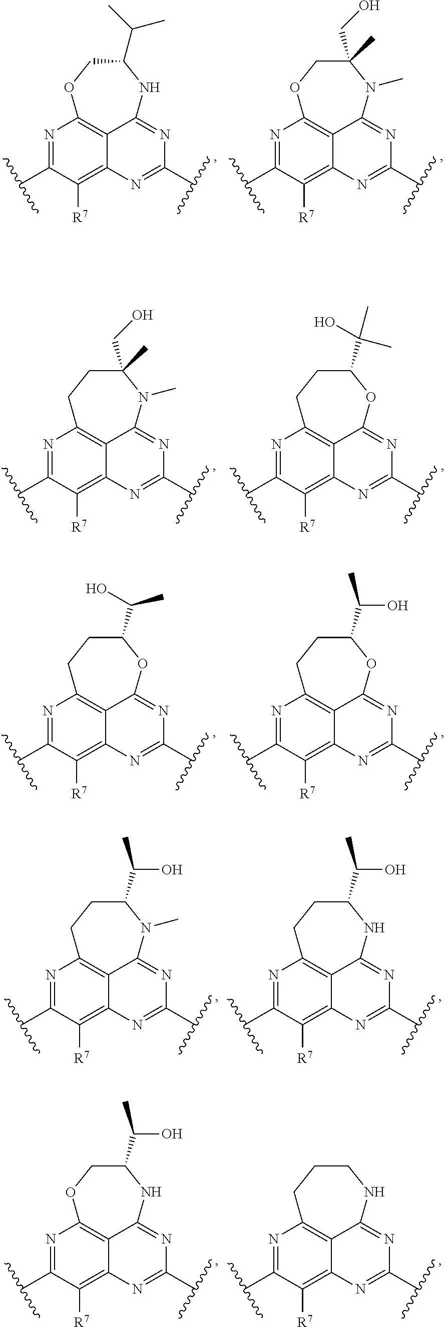

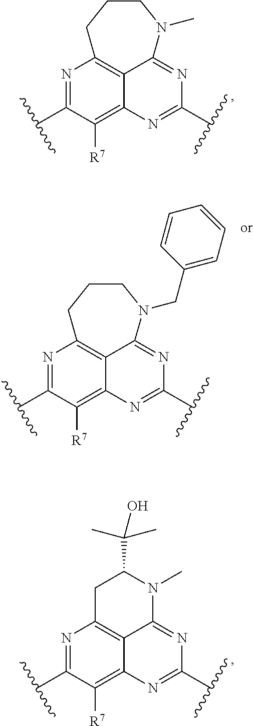

wherein each wherein “ [symbol] ” is a point of covalent attachment to either ring B or —(X)ₚ—R³.

In some embodiments, ring A is optionally taken together with ring C or ring D to form a core of the compound of the formula 1, the core having the formula

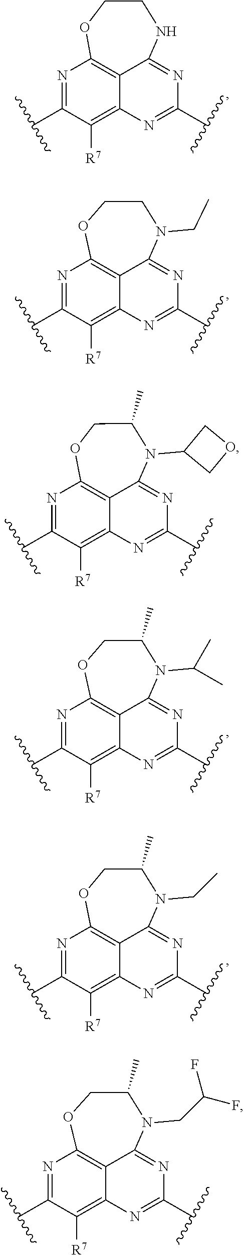

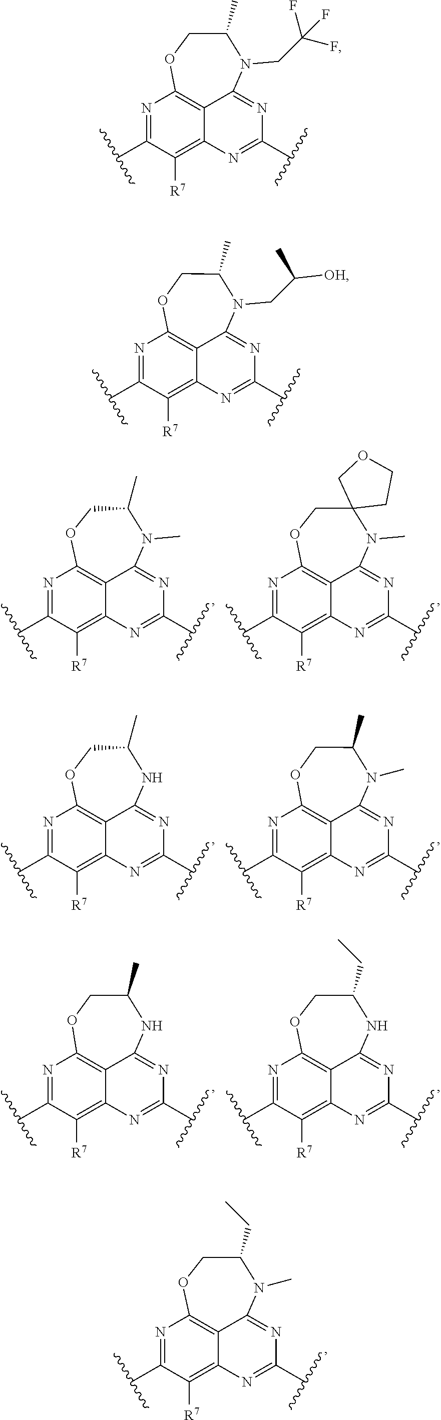

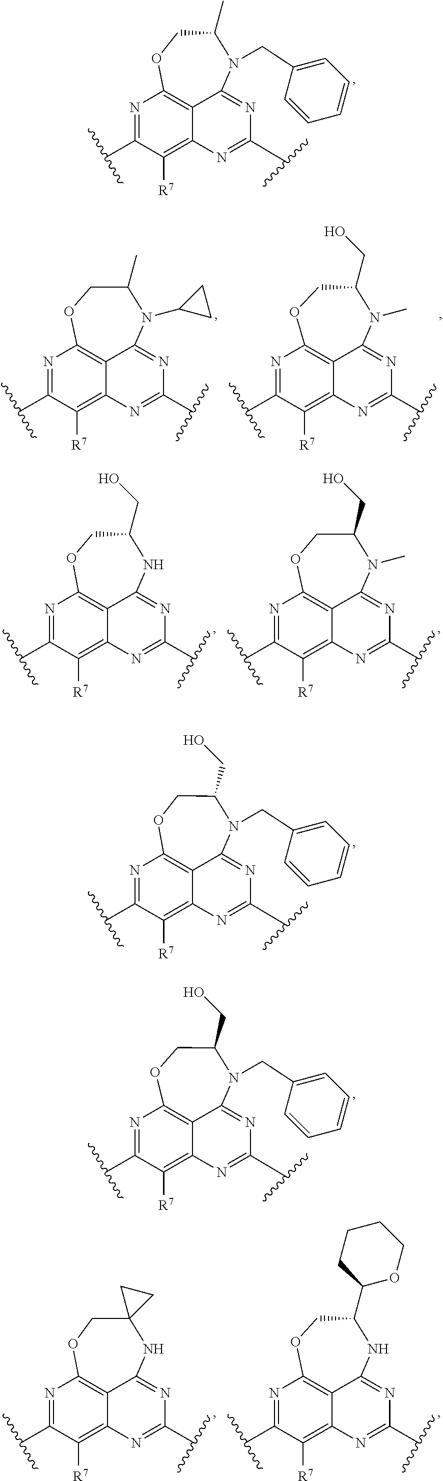

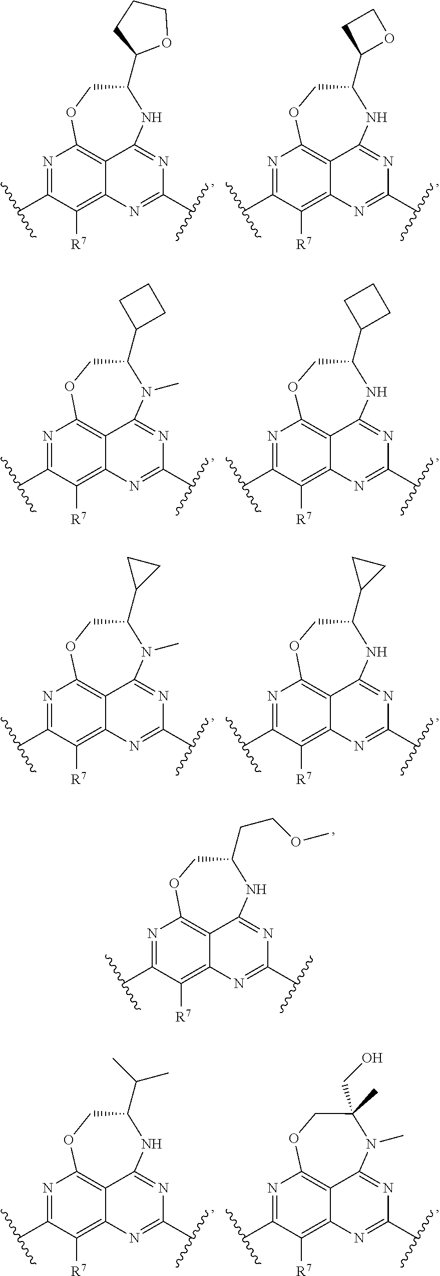

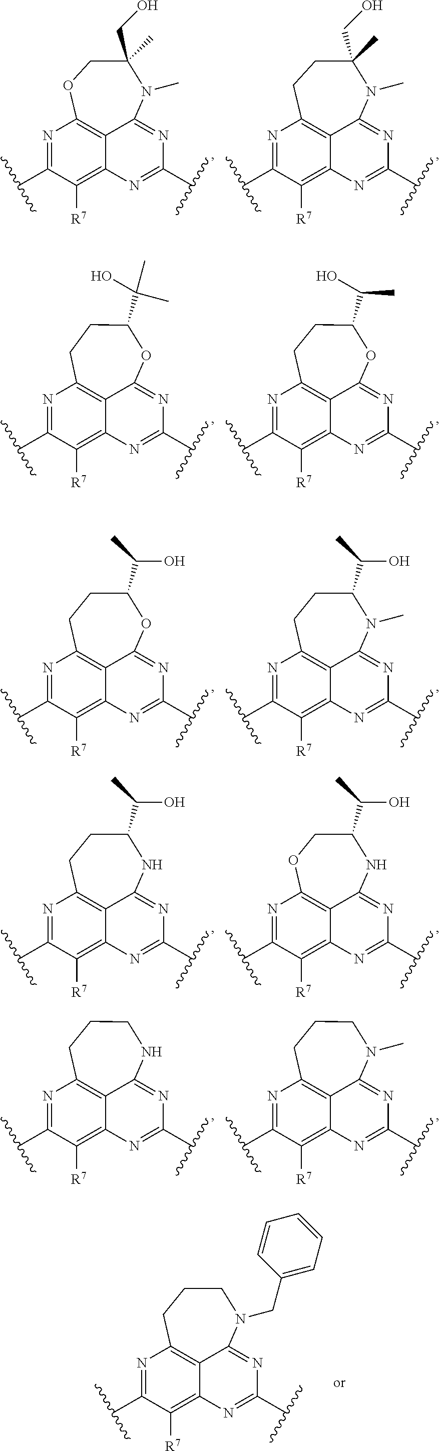

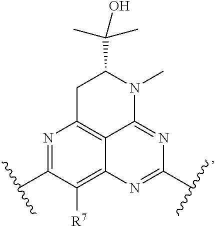

wherein each wherein “ [symbol] ” is a point of covalent attachment to either ring B or —(X)ₚ—R³.

In some embodiments, m is 0. In some embodiments, m is one or more. In some embodiments, m is 0, 1, 2, 3, 4, 5, 6, 7, or 8. In some embodiments, m is 0, 1, 2, 3, 4, 5, 6, or 7. In some embodiments, m is 0, 1, 2, 3, 4, 5, or 6. In some embodiments, m is 0, 1, 2, 3, 4, or 5. In some embodiments, m is 0, 1, 2, 3, or 4. In some embodiments, m is 0, 1, 2, or 3. In some embodiments, m is 0, 1, or 2. In some embodiments, m is 0 or 1. In some embodiments, m is 1. In some embodiments, m is 2. In some embodiments, m is 3. In some embodiments, m is 4.

In some embodiments, ring B is a C₆-C₁₀ aryl or 5- to 10-membered heteroaryl, optionally substituted with one or more R². In some embodiments, ring B is a C₃-C₆ cycloalkyl, 4- to 10-membered heterocycloalkyl, C₆-C₁₀ aryl, or 5- to 10-membered heteroaryl optionally substituted with one or more R². In some embodiments, ring B is a C₆-C₁₀ aryl, optionally substituted with one or more R². In some embodiments, ring B is a 5- to 10-membered heteroaryl, optionally substituted with one or more R².

In some embodiments, Ring B is

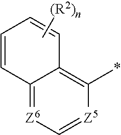

wherein * is a point of covalent attachment to

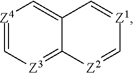

n is 0 or one or more. In some embodiments, n is 0, 1, 2, 3, 4, 5, 6, or 7.

In some embodiments, Ring B is

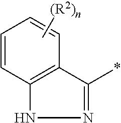

wherein * is a point of covalent attachment to

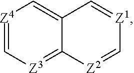

n is 0 or one or more. In some embodiments, n is 0, 1, 2, 3, or 4.

In some embodiments, Ring B is

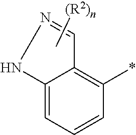

wherein * is a point of covalent attachment to

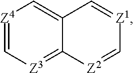

n is 0 or one or more. In some embodiments, n is 0, 1, 2, 3, or 4.

In some embodiments, Ring B is

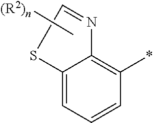

wherein * is a point of covalent attachment to

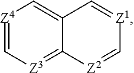

n is 0 or one or more. In some embodiments, n is 0, 1, 2, 3, or 4.

In some embodiments, Ring B is

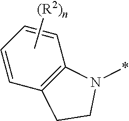

wherein * is a point of covalent attachment to

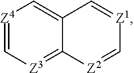

n is 0 or one or more. In some embodiments, n is 0, 1, 2, 3, or 4.

In some embodiments, Ring B is

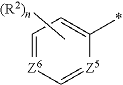

wherein * is a point of covalent attachment to

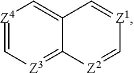

n is 0 or one or more. In some embodiments, n is 0, 1, 2, 3, or 4.

In some embodiments, Ring B is naphthyl, wherein each hydrogen atom in naphthyl is independently optionally substituted by R². In some embodiments, Ring B is phenyl, wherein each hydrogen atom in phenyl is independently optionally substituted by R². In some embodiments, Ring B is pyridyl, wherein each hydrogen atom in pyridyl is independently optionally substituted by R². In some embodiments, Ring B is indolyl, wherein each hydrogen atom in indolyl is independently optionally substituted by R². In some embodiments, Ring B is indazolyl, wherein each hydrogen atom in indazolyl is independently optionally substituted by R². In some embodiments, Ring B is benzothiazolyl, wherein each hydrogen atom in benzothiazolyl is independently optionally substituted by R².

In some embodiments, Ring B is

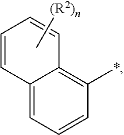

wherein * is a point of covalent attachment to

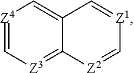

n is 0 or one or more. In some embodiments, n is 0, 1, 2, 3, 4, 5, 6, or 7.

In some embodiments, each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, each R¹ is deuterium, C₁-C₆ alkyl, C₂-C₆ alkenyl, or C₂-C₆ alkynyl, wherein each hydrogen atom in C₁-C₆ alkyl, C₂-C₆ alkenyl, or C₂-C₆ alkynyl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R^{g} and a R¹ or R¹⁰ and a R¹, taken together with the atoms to which they are attached, combine to form a 4- to 10-membered heterocycloalkyl, wherein each hydrogen atom in the 4- to 10-membered heterocycloalkyl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)₂C₁-C₆ alkyl, —P(O)₂OC₁-C₆ alkyl, —CN, or —NO₂. In some embodiments, two hydrogen atoms on a single carbon atom of the 4- to 10-membered heterocycloalkyl combine to form an oxo group or an C₂-C₆ alkenyl group; or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆alkyl, C₁-C₆haloalkyl, —R^{e}, —R^{e}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{c}R^{f}, —P(O)₂NR^{c}R^{f}, —P(O)OR^{c}, —P(O)₂OR^{c}, —CN, or —NO₂. In some embodiments, each R¹ is selected from the group consisting of deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R¹ and R¹⁰ taken together with the atom or atoms to which they are attached combine to form a monocyclic 4- to 10-membered heterocycloalkyl, a fused bicyclic 5- to 10-membered heterocycloalkyl, or a bridged bicyclic 6- to 10-membered heterocycloalkyl, wherein each hydrogen atom in the monocyclic 4- to 10-membered heterocycloalkyl, fused bicyclic 5- to 10-membered heterocycloalkyl, or a bridged bicyclic 6- to 10-membered heterocycloalkyl is independently optionally substituted by deuterium, halogen, C₁-C₆ alkyl, C₁-C₆haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or two hydrogen atoms on a single carbon atom of the monocyclic 4- to 10-membered heterocycloalkyl, fused bicyclic 5- to 10-membered heterocycloalkyl, or bridged bicyclic 6- to 10-membered heterocycloalkyl combine to form an oxo group or an alkenyl group.

In some embodiments, each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR¹R^{d}, —C(O)R¹, —C(O)OR¹, —C(O)NR¹R^{d}, —C(═NR^{e})NR¹R^{d}, —PR¹R^{d}, —P(O)R¹R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, each R² is independently deuterium, halogen, C₁-C₆ alkyl, C₂-C₆ alkenyl, C₂-C₆ alkynyl, —OR^{e}, —C(O)R^{c}, or —NR^{c}R^{d}. In some embodiments, each R² is independently deuterium, halogen, C₁-C₆ alkyl, C₂-C₆ alkenyl, C₂-C₆ alkynyl, —OR^{c}, or —CN. In some embodiments, each R² is independently deuterium, halogen, C₁-C₆ alkyl, C₂-C₆ alkenyl, C₂-C₆ alkynyl, —OR^{c}, —C(O)R^{c}, —NR^{c}R^{d}, or —CN, wherein each hydrogen atom in C₁-C₆ alkyl, C₂-C₆ alkenyl, and C₂-C₆ alkynyl is independently optionally substituted with a deuterium or halogen. In some embodiments, each R², when present, is independently selected from the group consisting of fluoro, chloro. C₁-C₆ alkyl, —OH, and —CN. In some embodiments, each R² is independently selected from the group consisting of fluoro, chloro, methyl, ethyl, iso-propyl, —NH₂, —C≡CH, —CN, —CF₃ and —OH. In some embodiments, each R², when present, is independently selected from the group consisting of fluoro, chloro, methyl, ethyl, iso-propyl, —C≡CH, —CN, and —OH.

In some embodiments, n is 0. In some embodiments, n is one or more. In some embodiments, n is 0, 1, 2, 3, 4, 5, 6, 7, or 8. In some embodiments, n is 0, 1, 2, 3, 4, 5, 6, or 7. In some embodiments, n is 0, 1, 2, 3, 4, 5, or 6. In some embodiments, n is 0, 1, 2, 3, 4, or 5. In some embodiments, n is 0, 1, 2, 3, or 4. In some embodiments, n is 0, 1, 2, or 3. In some embodiments, n is 1, 2, or 3. In some embodiments, n is 1, 2, 3, 4, or 5. In some embodiments, n is 2, 3, 4, or 5. In some embodiments, n is 0, 1, 2, 3, or 4. In some embodiments, n is 0, 1, or 2. In some embodiments, n is 2 or 3. In some embodiments, n is 0 or 1. In some embodiments, n is 1. In some embodiments, n is 2. In some embodiments, n is 3. In some embodiments, n is 4.

In some embodiments, Ring B is

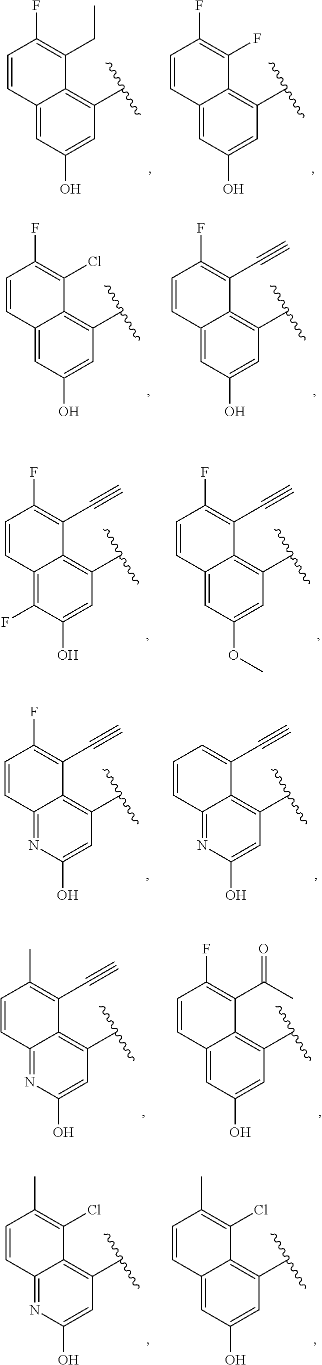

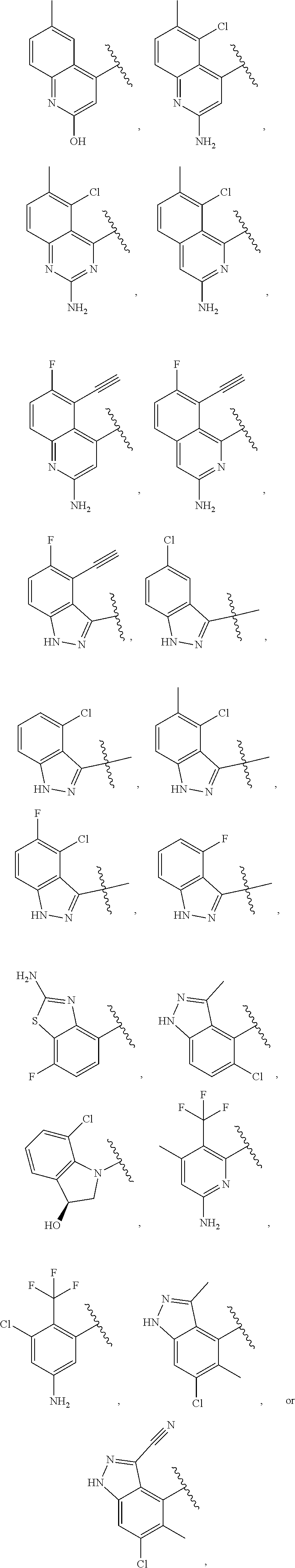

wherein “ [symbol] ” is a point of covalent attachment.

In some embodiments, Ring B is of the formula

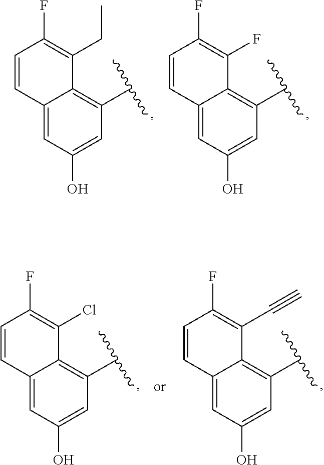

wherein “ [symbol] ” is a point of covalent attachment.

In some embodiments, Ring B is

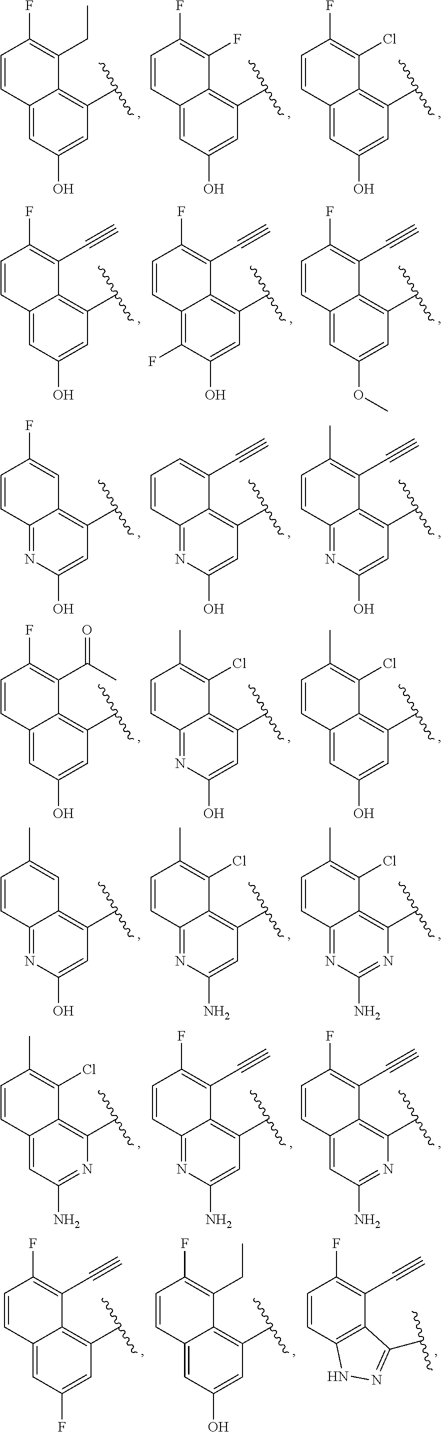

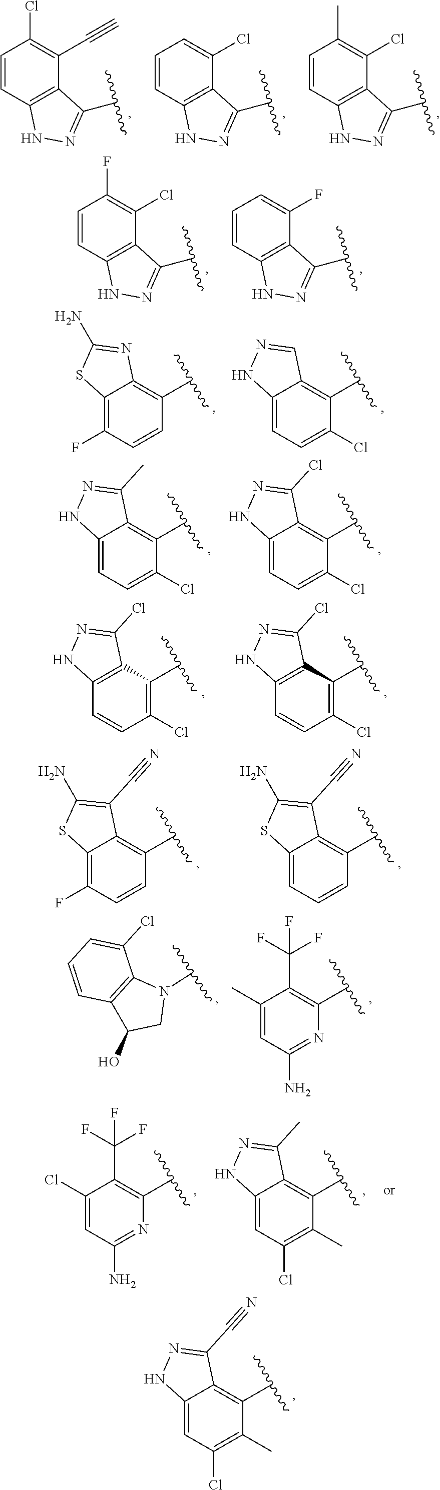

wherein “ [symbol] ” is a point of covalent attachment.

In some embodiments, p is 0. In some embodiments, p is 1.

In some embodiments, —X— is —O—, —S—, or —NR⁴—. In some embodiments, —X— is —O—. In some embodiments, —X— is —S—. In some embodiments, —X— is —NR⁴—.

In some embodiments, R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R¹, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R⁴ is H, deuterium, or C₁-C₆ alkyl. In some embodiments, R⁴ is H, deuterium, or methyl.

In some embodiments, R³ is —C₁-C₆ alkyl, —C₂-C₆ alkenyl, —C₂-C₆ alkynyl, —C₃-C₆ cycloalkyl, 4- to 10-membered heterocycloalkyl, —C₁-C₆ alkylene-(4- to 10-membered heterocycloalkyl), —C₆-C₁₀ aryl, —C₁-C₆ alkylene-(C₆-C₁₀ aryl), 5- to 10-membered heteroaryl, or —C₁-C₆ alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R³ is —C₁-C₆ alkyl, 4- to 10-membered heterocycloalkyl, or —C₁-C₆alkylene-(4- to 10-membered heterocycloalkyl), wherein each hydrogen atom in —C₁-C₆ alkyl, 4- to 10-membered heterocycloalkyl, or —C₁-C₆ alkylene-(4- to 10-membered heterocycloalkyl), is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆alkylene-(C₆-C₁₀ aryl), haloalkyl, C₃-C₆cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R³ is 4- to 10-membered heterocycloalkyl, wherein each hydrogen atom in 4- to 10-membered heterocycloalkyl is independently optionally substituted by deuterium, halogen, C₁-C₆ alkyl, —C₁-C₆ alkylene-O—C₁-C₆alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R³ is

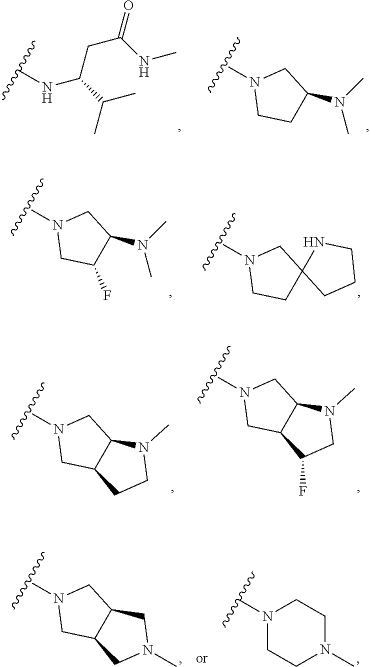

wherein “ [symbol] ” is a point of covalent attachment.

In some embodiments, R³ is

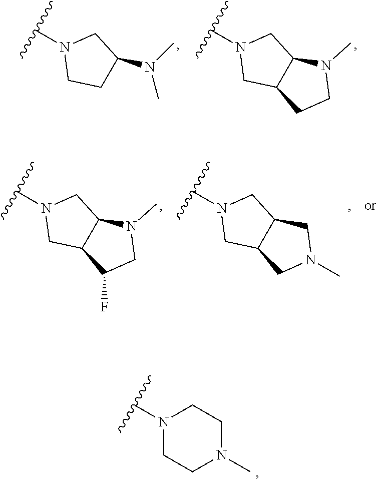

wherein “ [symbol] ” is a point of covalent attachment.

In some embodiments, R³ is

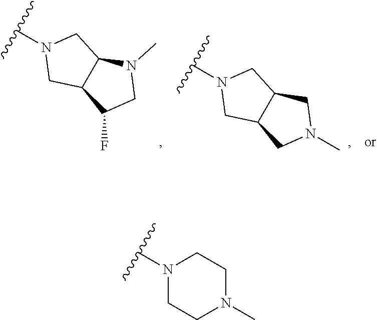

wherein “ [symbol] ” is a point of covalent attachment.

In some embodiments, R³ is —C₁-C₆ alkylene-(4- to 10-membered heterocycloalkyl), wherein each hydrogen atom in methyl, ethyl, propyl, or —C₁-C₆ alkylene-(4- to 10-membered heterocycloalkyl), is independently optionally substituted by deuterium, halogen, C₁-C₆ alkyl, —C₁-C₆ alkylene-O—C₁-C₆ alkyl, —OC₁-C₆ alkylene-O—C₁-C₈alkyl, —C₁-C₆ alkylene-O—R^{a}, C₆-C₁₀ aryl, —C₁-C₆alkylene-(C₆-C₁₀ aryl), haloalkyl, C₃-C₆cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R³ is —C₁-C₆ alkylene-(4- to 10-membered heterocycloalkyl), wherein each hydrogen atom in —C₁-C₆ alkylene-(4- to 10-membered heterocycloalkyl), is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R³ is of the formula

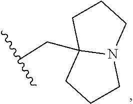 [SMILES: *CC12CCCN1CCC2]

wherein “ [symbol] ” is a point of covalent attachment, and each hydrogen atom is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{c}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R³ is of the formula

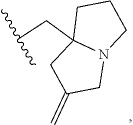 [SMILES: *CC12CCCN1CC(=C)C2]

wherein “ [symbol] ” is a point of covalent attachment, and each hydrogen atom is [symbol] independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R³ is of the formula

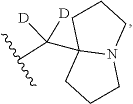 [SMILES: *C([2H])([2H])C12CCCN1CCC2]

wherein “ [symbol] ” is a point of covalent attachment, and each hydrogen atom is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R³ is of the formula

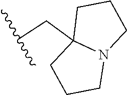 [SMILES: *CC12CCCN1CCC2]

wherein “ [symbol] ” is a point of covalent attachment, and each hydrogen atom is independently optionally substituted by deuterium, halogen, C₁-C₆ alkyl, haloalkyl, —OR^{e}, —CN, or —NO₂.

In some embodiments, R³ is of the formula

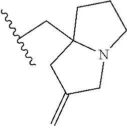 [SMILES: *CC12CCCN1CC(=C)C2]

wherein “ [symbol] ” is a point of covalent attachment, and each hydrogen atom is independently optionally substituted by deuterium, halogen, C₁-C₆ alkyl, —OR^{e}, —CN, or —NO₂.

In some embodiments R³ is of the formula

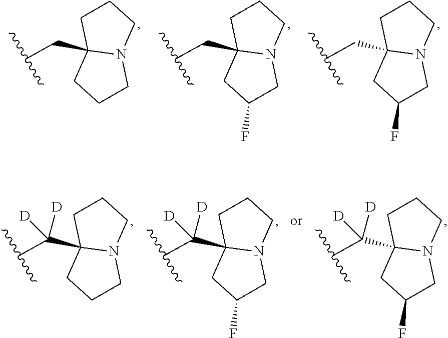

wherein “ [symbol] ” is a point of covalent attachment.

In some embodiments R³ is of the formula

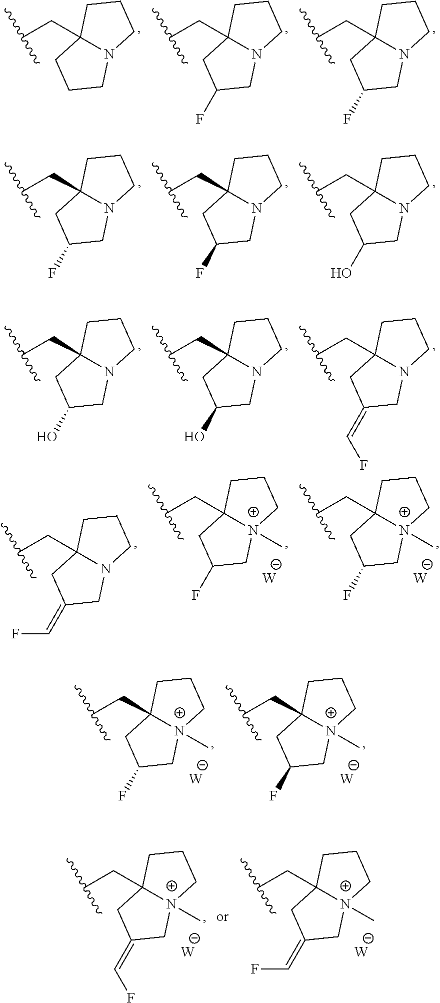

wherein W^{⊖} is an inorganic counter ion or an organic counter ion.

In some embodiments, R³ is of the formula

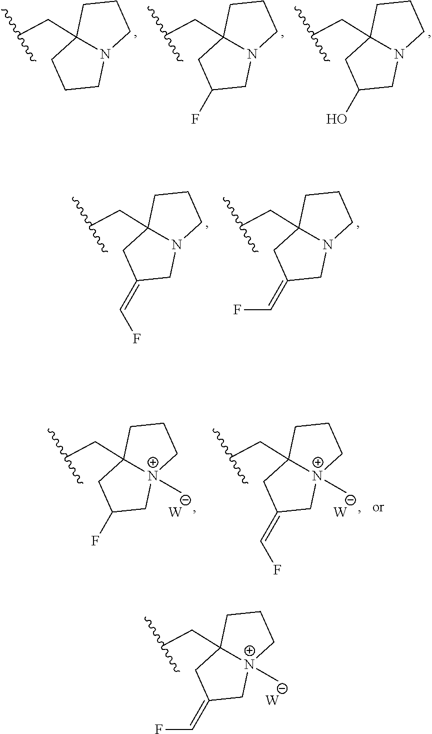

wherein W^{⊖} is an inorganic counter ion or an organic counter ion.

In some embodiments, each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{c} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₅-C₁₀ aryl, and 5- to 10-membered heteroaryl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or R^{c} and R^{f} taken together with the carbon atom to which they are attached form an oxo groups or an alkenyl.

In some embodiments, each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl.

In some embodiments, Z¹ is N. In some embodiments, Z² is N. In some embodiments, Z³ is N. In some embodiments, Z⁴ is N. In some embodiments, Z⁵ is N. In some embodiments, Z⁶ is N. In some embodiments, Z¹ is C(R′). In some embodiments, Z² is C(R⁶). In some embodiments, Z³ is C(R⁷). In some embodiments, Z³ is CH. In some embodiments, Z³ is CF. In some embodiments, Z⁴ is C(R′). In some embodiments, Z⁵ is C(R¹⁵). In some embodiments, Z⁶ is C(R¹⁶). In some embodiments, Z⁵ is CH. In some embodiments, Z⁶ is CH. In some embodiments, any of the possible combinations of Z¹-Z⁷ can be combined as embodiments. In some embodiments, Z⁵ is N or C(R¹⁵). In some embodiments, Z⁶ is N or C(R¹⁶). In some embodiments, Z¹ is N, and Z² is N. In some embodiments, Z¹ is N, Z² is N, Z³ is C(R⁷), and Z⁴ is N. In some embodiments, Z¹ is N, Z² is N, Z³ is C(R⁷), and Z⁴ is C(R⁸). In some embodiments, Z¹ is N, Z² is N, Z³ is N, and Z⁴ is N. In some embodiments, Z¹ is N, Z² is N, Z³ is N, and Z⁴ is C(R⁸). In some embodiments, Z¹ is N, Z² is N, Z³ is C(R⁷), and Z⁴ is C(R⁸). In some embodiments, Z¹ is N, Z² is N, Z³ is CH, and Z⁴ is N. In some embodiments, Z¹ is N, Z² is N, Z³ is CF, and Z⁴ is N.

In some embodiments, each of R⁵, R⁶, R⁷, R¹, R¹⁵, and R¹⁶ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂. In some embodiments, each of R⁵, R⁶, R⁷, R⁸, R¹⁵, and R¹⁶ is independently H, deuterium, halogen, C₁-C₆ alkyl, C₂-C₆ alkenyl, C₂-C₆ alkynyl, C₃-C₆ cycloalkyl, —OR^{g}, —CN, or —NO₂. In some embodiments, each of R⁵, R⁶, R⁷, R¹, R¹⁵, and R¹⁶ is independently H, deuterium, halogen, C₁-C₆ alkyl, —OR^{g}, —CN, or —NO₂.

In some embodiments, each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, —OR^{g}, —CN, or —NO₂.

In some embodiments, each of R¹⁵ and R¹⁶ is independently H, deuterium, halogen, C₁-C₆alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR⁸R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂; In some embodiments, each of R¹⁵ and R¹⁶ is independently H, deuterium, halogen, C₁-C₆ alkyl, C₂-C₆ alkenyl, C₂-C₆ alkynyl, C₃-C₆ cycloalkyl, —OR^{g}, —CN, or —NO₂.

In some embodiments, R⁵, when present, is H. In some embodiments, R⁶, when present, is H. In some embodiments, R⁷, when present, is H or F. In some embodiments, R⁸, when present, is H. In some embodiments, R¹⁵, when present, is H. In some embodiments, R¹⁶, when present, is H.

In some embodiments, the disclosure provides a compound of the formula

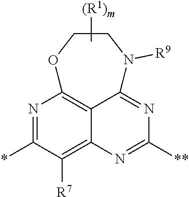

wherein
* represents the point of covalent attachment to Ring B as described herein;
** represents the point of covalent attachment to —(X)ₚ—R³ as described herein; and
R¹, R⁷, R⁹ and m are as described herein.

In some embodiments, the disclosure provides a compound of the formula

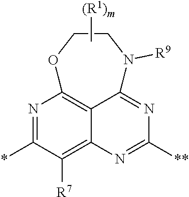

wherein
* represents the point of covalent attachment to Ring B as described herein;
** represents the point of covalent attachment to —(X)ₚ—R³ as described herein;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆haloalkyl, —R^{e}, —R¹, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁷ is H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
R^{g} is H, deuterium, —C(O)R^{g}, —C(O)NR^{g}R^{h}, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{c}, —OC(O)R^{e}, —OC(O)NR^{e}R^{f}, —OS(O)R^{e}, —OS(O)₂R^{e}, —OS(O)NR^{e}R^{f}, —OS(O)₂NR^{c}R^{f}, —SR^{c}, —S(O)R^{c}, —S(O)₂R^{c}. —S(O)NR^{c}R^{f}, —S(O)₂NR^{c}R^{f}, —NR^{c}R^{f}, —NR^{c}C(O)R^{f}, —NR^{c}C(O)OR^{f}, —NR^{c}C(O)NR^{e}R^{f}, —NRS(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and

m is 0, 1, or 2.

In some embodiments, the disclosure provides a compound of the formula

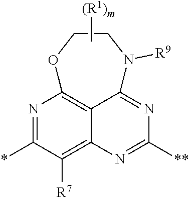

wherein
* represents the point of covalent attachment to Ring B as described herein;
** represents the point of covalent attachment to —(X)ₚ—R³ as described herein;
each R¹ is independently deuterium, halogen, C₁-C₆ alkyl, C₃-C₅ cycloalkyl, or 4- to 10-membered heterocycloalkyl, wherein each hydrogen atom in C₁-C₆ alkyl, C₃-C₆ cycloalkyl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁷ is H, deuterium, or halogen; and
R⁹ is H, —C(O)R^{g}, —C(O)NR^{g}R^{h}, C₁-C₆ alkyl, or C₃-C₆ cycloalkyl, wherein each hydrogen atom in C₁-C₆ alkyl and C₃-C₆ cycloalkyl is independently optionally substituted by deuterium, halogen, —R^{e}, or —OR^{c}; and

wherein m is 0, 1, or 2.

In some embodiments, the disclosure provides a compound selected from the group consisting of 5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
5-ethyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
(8aR,9R)-5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7-oxa-1,3,6,12a-tetraazabenzo[4,5]cyclohepta[1,2,3-de]naphthalen-9-ol;
(8aR,9R)-5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7H-1,3,6,12a-tetraazabenzo[4,5]cyclohepta[1,2,3-de]naphthalen-9-ol;
5-ethynyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-9,10-dimethyl-9,10-dihydro-8H-7-oxa-1,3,6,10-tetraazacyclohepta[1,2,3-de]naphthalen-5-yl]naphthalen-2-ol;
5-ethynyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-9,10-dimethyl-9,10-dihydro-8H-7-oxa-1,3,6,10-tetraazacyclohepta[1,2,3-de]naphthalen-5-yl]naphthalen-2-ol;
5-ethyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-9,10-dimethyl-9,10-dihydro-8H-7-oxa-1,3,6,10-tetraazacyclohepta[1,2,3-de]naphthalen-5-yl]naphthalen-2-ol;
5-ethynyl-6-fluoro-4-[(9R)-4-fluoro-2-{[(2R,7aS)-2-fluorotetrahydro-1H-pyrrolizin-7a(5H)-yl]methoxy)}-9-(hydroxymethyl)-9,10-dimethyl-7,8,9,10-tetrahydro-1,3,6,10-tetraazacyclohepta[1,2,3-de]naphthalen-5-yl]naphthalen-2-ol;
5-ethyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-9,10-dimethyl-7,8,9,10-tetrahydro-1,3,6,10-tetraazacyclohepta[1,2,3-de]naphthalen-5-yl]naphthalen-2-ol;
5-ethynyl-6-fluoro-4-[(9R)-4-fluoro-2-{[(2R,7aS)-2-fluorotetrahydro-1H-pyrrolizin-7a(5H)-yl]methoxy}-9-(2-hydroxypropan-2-yl)-8,9-dihydro-7H-10-oxa-1,3,6-triazacyclohepta[1,2,3-de]naphthalen-5-yl]naphthalen-2-ol;
5-ethyl-6-fluoro-4-[(9R)-4-fluoro-2-{[(2R,7aS)-2-fluorotetrahydro-1H-pyrrolizin-7a(5H)-yl]methoxy}-9-(2-hydroxypropan-2-yl)-8,9-dihydro-7H-10-oxa-1,3,6-triazacyclohepta[1,2,3-de]naphthalen-5-yl]naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(1S)-1-hydroxyethyl]-8,9-dihydro-7H-10-oxa-1,3,6-triazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1S)-1-hydroxyethyl]-8,9-dihydro-7H-10-oxa-1,3,6-triazacyclohepta[1,2,3-de]naphthalen-5-yl}naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-8,9-dihydro-7H-10-oxa-1,3,6-triazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-8,9-dihydro-7H-10-oxa-1,3,6-triazacyclohepta[1,2,3-de]naphthalen-5-yl}naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-10-methyl-7,8,9,10-tetrahydro-1,3,6,10-tetraazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-10-methyl-7,8,9,10-tetrahydro-1,3,6,10-tetraazacyclohepta[1,2,3-de]naphthalen-5-yl}naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-7,8,9,10-tetrahydro-1,3,6,10-tetraazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-7,8,9,10-tetrahydro-1,3,6,10-tetraazacyclohepta[1,2,3-de]naphthalen-5-yl}naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-9,10-dihydro-8H-7-oxa-1,3,6,10-tetraazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-9,10-dihydro-8H-7-oxa-1,3,6,10-tetraazacyclohepta[1,2,3-de]naphthalen-5-yl}naphthalen-2-ol;
5-ethynyl-6-fluoro-4-[(5R)-9-fluoro-2-{[(2R,7aS)-2-fluorotetrahydro-1H-pyrrolizin-7a(5H)-yl]methoxy}-5-(2-hydroxypropan-2-yl)-4-methyl-5,6-dihydro-4H-pyrimido[4,5,6-de][1,6]naphthyridin-8-yl]naphthalen-2-ol;
5-ethyl-6-fluoro-4-[(5R)-9-fluoro-2-{[(2R,7aS)-2-fluorotetrahydro-1H-pyrrolizin-7a(5H)-yl]methoxy}-5-(2-hydroxypropan-2-yl)-4-methyl-5,6-dihydro-4H-pyrimido[4,5,6-de][1,6]naphthyridin-8-yl]naphthalen-2-ol;
5-chloro-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-7,8,9,10-tetrahydro-1,3,6,10-tetraazacyclohepta[1,2,3-de]naphthalen-5-yl}naphthalen-2-ol;
5,6-difluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-7,8,9,10-tetrahydro-1,3,6,10-tetraazacyclohepta[1,2,3-de]naphthalen-5-yl}naphthalen-2-ol;
(8aR,9R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol;
(8aR,9R)-5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol;
5-ethynyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-10-methyl-9,10-dihydro-8H-7-oxa-1,3,6,10-tetraazacyclohepta[1,2,3-de]naphthalen-5-yl]naphthalen-2-ol;
5-ethyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-10-methyl-9,10-dihydro-8H-7-oxa-1,3,6,10-tetraazacyclohepta[1,2,3-de]naphthalen-5-yl]naphthalen-2-ol;
(8aS,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-11-ol;
(8aS,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-11-methyl-8,8a,9,10,11,12-hexahydro-7-oxa-1,3,6,12a-tetraazabenzo[4,5]cyclohepta[1,2,3-de]naphthalen-11-ol;
4-[(9S)-9-cyclopropyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethynyl-6-fluoronaphthalen-2-ol;
4-[(9S)-9-cyclopropyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-5-ethynyl-6-fluoronaphthalen-2-ol;
4-[(9S)-9-cyclopropyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethyl-6-fluoronaphthalen-2-ol;
4-[(9S)-9-cyclopropyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-5-ethyl-6-fluoronaphthalen-2-ol;
4-[(9S)-9-cyclobutyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethynyl-6-fluoronaphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(2R)-oxetan-2-yl]-9,10-dihydro-8H-7-oxa-1,3,6,10-tetraazacyclohepta[1,2,3-de]naphthalen-5-yl}naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(2R)-oxolan-2-yl]-9,10-dihydro-8H-7-oxa-1,3,6,10-tetraazacyclohepta[1,2,3-de]naphthalen-5-yl}naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(2R)-oxan-2-yl]-9,10-dihydro-8H-7-oxa-1,3,6,10-tetraazacyclohepta[1,2,3-de]naphthalen-5-yl}naphthalen-2-ol;
5-ethyl-6-fluoro-4-[(8S,8aS)-4-fluoro-2-{[(2R,7aS)-2-fluorotetrahydro-1H-pyrrolizin-7a(5H)-yl]methoxy}-8-methyl-8,8a,9,10,11,12-hexahydro-7-oxa-1,3,6,12a-tetraazabenzo[4,5]cyclohepta[1,2,3-de]naphthalen-5-yl]naphthalen-2-ol;
7-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-1,3-benzothiazol-2-amine;
5-ethyl-6-fluoro-4-[(8aS)-4-fluoro-2-{[(2Z,7aS)-2-(fluoromethylidene)tetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
(3S)-7-chloro-1-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-2,3-dihydro-1H-indol-3-ol;
6-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-4-methyl-5-(trifluoromethyl)pyridin-2-amine;
3-chloro-5-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-4-(trifluoromethyl)aniline;
5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydropyrido[2′,1′:3,4][1,4]oxazepino[5,6,7-de]quinazolin-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-methyl-9,10-dihydro-8H-7-oxa-1,3,6,10-tetraazacyclohepta[1,2,3-de]naphthalen-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-(propan-2-yl)-9,10-dihydro-8H-7-oxa-1,3,6,10-tetraazacyclohepta[1,2,3-de]naphthalen-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-(2-methoxyethyl)-9,10-dihydro-8H-7-oxa-1,3,6,10-tetraazacyclohepta[1,2,3-de]naphthalen-5-yl]naphthalen-2-ol;
4-(9-cyclopropyl-4-fluoro-2-(((2R,7aS)-2-fluorotetrahydro-1H-pyrrolizin-7a(5H)-yl)methoxy)-10-methyl-9,10-dihydro-8H-7-oxa-1,3,6,10-tetraazacyclohepta[de]naphthalen-5-yl)-5-ethynyl-6-fluoronaphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9,10-dimethyl-9,10-dihydro-8H-7-oxa-1,3,6,10-tetraazacyclohepta[1,2,3-de]naphthalen-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-10-methyl-9-(propan-2-yl)-9,10-dihydro-8H-7-oxa-1,3,6,10-tetraazacyclohepta[1,2,3-de]naphthalen-5-yl]naphthalen-2-ol;
5-ethynyl-6-fluoro-4-[(8aS)-4-fluoro-2-{[(2Z,7aS)-2-(fluoromethylidene)tetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
(8aR,9R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7-oxa-1,3,6,12a-tetraazabenzo[4,5]cyclohepta[1,2,3-de]naphthalen-9-ol;
5-ethynyl-6-fluoro-4-[(7aR,10aS)-4-fluoro-2-{[(2R,7aS)-2-fluorotetrahydro-1H-pyrrolizin-7a(5H)-yl]methoxy}-11-methyl-7a,8,10a,11-tetrahydro-10H-7,9-dioxa-1,3,6,11-tetraazanaphtho[1,8-fg]azulen-5-yl]naphthalen-2-ol;
5-ethynyl-6-fluoro-4-[(7aS,9aS)-4-fluoro-2-{[(2R,7aS)-2-fluorotetrahydro-1H-pyrrolizin-7a(5H)-yl]methoxy}-10-methyl-8,9,9a,10-tetrahydro-7aH-7-oxa-1,3,6,10-tetraazacyclobuta[5,6]cyclohepta[1,2,3-de]naphthalen-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-methyl-10-(propan-2-yl)-9,10-dihydro-8H-7-oxa-1,3,6,10-tetraazacyclohepta[1,2,3-de]naphthalen-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-methyl-10-(oxetan-3-yl)-9,10-dihydro-8H-7-oxa-1,3,6,10-tetraazacyclohepta[1,2,3-de]naphthalen-5-yl]naphthalen-2-ol;
5-ethynyl-6-fluoro-4-[(8aS)-4-fluoro-2-{[(2R,7aS)-2-fluorotetrahydro-1H-pyrrolizin-7a(5H)-yl]methoxy}-8a,9,11,12-tetrahydro-8H-7,10-dioxa-1,3,6,12a-tetraazabenzo[4,5]cyclohepta[1,2,3-de]naphthalen-5-yl]naphthalen-2-ol;
5-ethynyl-6-fluoro-4-[(8aS,12S)-4-fluoro-2-{[(2R,7aS)-2-fluorotetrahydro-1H-pyrrolizin-7a(5H)-yl]methoxy}-12-methyl-8a,9,11,12-tetrahydro-8H-7,10-dioxa-1,3,6,12a-tetraazabenzo[4,5]cyclohepta[1,2,3-de]naphthalen-5-yl]naphthalen-2-ol;
(8aS,12S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8a,9,11,12-tetrahydro-8H-7,10-dioxa-1,3,6,12a-tetraazabenzo[4,5]cyclohepta[1,2,3-de]naphthalene-12-carbonitrile;
(8aS,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8a,9,11,12-tetrahydro-8H-7,10-dioxa-1,3,6,12a-tetraazabenzo[4,5]cyclohepta[1,2,3-de]naphthalene-11-carbonitrile;
(8aS,12R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-12-carbonitrile;
(8aS,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-11-carbonitrile;
5-ethynyl-6-fluoro-4-[(8aS)-4-fluoro-2-{[(2R,7aS)-2-fluorotetrahydro-1H-pyrrolizin-7a(5H)-yl]methoxy}-8a,9,12,13-tetrahydro-8H,11H-7,10-dioxa-1,3,6,13a-tetraazanaphtho[1,8-ab]heptalen-5-yl]naphthalen-2-ol;
5-ethynyl-6-fluoro-4-[(8aS,13S)-4-fluoro-2-{[(2R,7aS)-2-fluorotetrahydro-1H-pyrrolizin-7a(5H)-yl]methoxy}-13-methyl-8a,9,12,13-tetrahydro-8H,11H-7,10-dioxa-1,3,6,13a-tetraazanaphtho[1,8-ab]heptalen-5-yl]naphthalen-2-ol;
(8aS,13R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8a,9,12,13-tetrahydro-8H,11H-7,10-dioxa-1,3,6,13a-tetraazanaphtho[1,8-ab]heptalene-13-carbonitrile;
5-ethynyl-6-fluoro-4-[(8aS,12S)-4-fluoro-2-{[(2R,7aS)-2-fluorotetrahydro-1H-pyrrolizin-7a(5H)-yl]methoxy}-12-methyl-8a,9,12,13-tetrahydro-8H,11H-7,10-dioxa-1,3,6,13a-tetraazanaphtho[1,8-ab]heptalen-5-yl]naphthalen-2-ol;
(8aS,12R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8a,9,12,13-tetrahydro-8H,11H-7,10-dioxa-1,3,6,13a-tetraazanaphtho[1,8-ab]heptalene-12-carbonitrile;
5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10,12,13-hexahydro-7,11-dioxa-1,3,6,13a-tetraazanaphtho[1,8-ab]heptalen-5-yl]naphthalen-2-ol;
5-ethynyl-6-fluoro-4-[(8aS,9R)-4-fluoro-2-{[(2R,7aS)-2-fluorotetrahydro-1H-pyrrolizin-7a(5H)-yl]methoxy}-9-methyl-8,8a,9,10,12,13-hexahydro-7,11-dioxa-1,3,6,13a-tetraazanaphtho[1,8-ab]heptalen-5-yl]naphthalen-2-ol; and
5-ethynyl-6-fluoro-4-[(8aS,13S)-4-fluoro-2-{[(2R,7aS)-2-fluorotetrahydro-1H-pyrrolizin-7a(5H)-yl]methoxy}-13-methyl-8,8a,9,10,12,13-hexahydro-7,11-dioxa-1,3,6,13a-tetraazanaphtho[1,8-ab]heptalen-5-yl]naphthalen-2-ol;
   or a pharmaceutically acceptable salt thereof.

In some embodiments, the disclosure provides a compound selected from the group consisting of
4-[(9S)-10-benzyl-4-fluoro-2-{[(2R,7aS)-2-fluorotetrahydro-1H-pyrrolizin-7a(5H)-yl]methoxy}-9-methyl-9,10-dihydro-8H-7-oxa-1,3,6,10-tetraazacyclohepta[1,2,3-de]naphthalen-5-yl]-5-ethynyl-6-fluoronaphthalen-2-ol;
5-ethynyl-6-fluoro-4-[(8aS)-4-fluoro-2-{[(2Z,7aS)-2-(fluoromethylidene)tetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
(2R,7aS)-7a-({[(9S)-5-(8-ethynyl-7-fluoro-3-hydroxynaphthalen-1-yl)-4-fluoro-9-methyl-9,10-dihydro-8H-7-oxa-1,3,6,10-tetraazacyclohepta[1,2,3-de]naphthalen-2-yl]oxy}methyl)-2-fluoro-4-methylhexahydro-1H-pyrrolizin-4-ium;
(8aS)-5-(5-chloro-1H-indazol-4-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene;
5-ethynyl-6-fluoro-4-[(8aS)-4-fluoro-2-{[(2E)-2-(fluoromethylidene)tetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
4-[(9S)-10-benzyl-4-fluoro-2-{[(2R,7aS)-2-fluorotetrahydro-1H-pyrrolizin-7a(5H)-yl]methoxy}-9-(hydroxymethyl)-9,10-dihydro-8H-7-oxa-1,3,6,10-tetraazacyclohepta[1,2,3-de]naphthalen-5-yl]-5-ethynyl-6-fluoronaphthalen-2-ol;
4-[(9R)-10-benzyl-4-fluoro-2-{[(2R,7aS)-2-fluorotetrahydro-1H-pyrrolizin-7a(5H)-yl]methoxy}-9-(hydroxymethyl)-9,10-dihydro-8H-7-oxa-1,3,6,10-tetraazacyclohepta[1,2,3-de]naphthalen-5-yl]-5-ethynyl-6-fluoronaphthalen-2-ol;
5-ethynyl-6-fluoro-4-(4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl)naphthalen-2-ol;
(5P,8aS)-5-(3,5-dichloro-1H-indazol-4-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene;
(5M,8aS)-5-(3,5-dichloro-1H-indazol-4-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene;
2-amino-7-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-1-benzothiophene-3-carbonitrile;
(8aS,11S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-11-ol;
(8aR,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-11-ol;
6-((S)-1-fluoro-11-(((2R,7aS)-2-fluorotetrahydro-1H-pyrrolizin-7a(5H)-yl)methoxy)-5,5a,6,7,8,9-hexahydro-4-oxa-3,9a,10,12-tetraazabenzo[4,5]cyclohepta[1,2,3-de]naphthalen-2-yl)-4-methyl-5-(trifluoromethyl)pyridin-2-amine;
(8aS)-5-(8-ethynyl-3,7-difluoronaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene;
4-[(9S)-9-ethyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethynyl-6-fluoronaphthalen-2-ol;
5-ethynyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-10-methyl-9,10-dihydro-8H-7-oxa-1,3,6,10-tetraazacyclohepta[1,2,3-de]naphthalen-5-yl]naphthalen-2-ol;
5-ethyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-10-methyl-9,10-dihydro-8H-7-oxa-1,3,6,10-tetraazacyclohepta[1,2,3-de]naphthalen-5-yl]naphthalen-2-ol;
5-ethynyl-6-fluoro-4-(4′-fluoro-2′-{[(2R,7aS)-2-fluorotetrahydro-1H-pyrrolizin-7a(5H)-yl]methoxy}-10′-methyl-8′H,10′H-spiro[cyclopropane-1,9′-[7]oxa[1,3,6,10]tetraazacyclohepta[1,2,3-de]naphthalen]-5′-yl)naphthalen-2-ol;
(8aR,11S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-11-ol;
5-ethynyl-6-fluoro-4-(4′-fluoro-2′-{[(2R,7aS)-2-fluorotetrahydro-1H-pyrrolizin-7a(5H)-yl]methoxy}-8′H,10′H-spiro[cyclopropane-1,9′-[7]oxa[1,3,6,10]tetraazacyclohepta[1,2,3-de]naphthalen]-5′-yl)naphthalen-2-ol;
4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-5-ethynyl-6-fluoronaphthalen-2-ol;
(2R,7aS)-7a-({[(8aS)-5-(8-ethynyl-7-fluoro-3-hydroxynaphthalen-1-yl)-4-fluoro-8,8a,9,10,11,12-hexahydro-7-oxa-1,3,6,12a-tetraazabenzo[4,5]cyclohepta[1,2,3-de]naphthalen-2-yl]oxy}methyl)hexahydro-1H-pyrrolizin-2-ol;
5-ethynyl-6-fluoro-4-[(8aS)-4,11,11-trifluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
5-ethyl-4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-6-fluoronaphthalen-2-ol;
5-ethyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-9,10-dihydro-8H-7-oxa-1,3,6,10-tetraazacyclohepta[1,2,3-de]naphthalen-5-yl]naphthalen-2-ol;
(2S,7aS)-7a-({[(8aS)-5-(8-ethynyl-7-fluoro-3-hydroxynaphthalen-1-yl)-4-fluoro-8,8a,9,10,11,12-hexahydro-7-oxa-1,3,6,12a-tetraazabenzo[4,5]cyclohepta[1,2,3-de]naphthalen-2-yl]oxy}methyl)hexahydro-1H-pyrrolizin-2-ol;
5-ethynyl-6-fluoro-4-[(7aR,10aS)-4-fluoro-2-{[(2R,7aS)-2-fluorotetrahydro-1H-pyrrolizin-7a(5H)-yl]methoxy}-7a,8,9,10,10a,11-hexahydro-7-oxa-1,3,6,11-tetraazanaphtho[1,8-fg]azulen-5-yl]naphthalen-2-ol;
(8aS,9S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol;
5-ethynyl-6-fluoro-4-[4-fluoro-2-{[(2R,7aS)-2-fluorotetrahydro-1H-pyrrolizin-7a(5H)-yl]methoxy}-13-(hydroxymethyl)-8,9,10,11-tetrahydro-8,12-methano-7-oxa-1,3,6,12-tetraazacyclonona[1,2,3-de]naphthalen-5-yl]naphthalen-2-ol;
4-[(8aS)-4,11-difluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-5-ethynyl-6-fluoronaphthalen-2-ol;
5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,12-tetrahydro-7-oxa-1,3,6,12a-tetraazabenzo[4,5]cyclohepta[1,2,3-de]naphthalen-5-yl]naphthalen-2-ol;
5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10-tetrahydro-7-oxa-1,3,6,12a-tetraazabenzo[4,5]cyclohepta[1,2,3-de]naphthalen-5-yl]naphthalen-2-ol;
4-(10-ethyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl)-5-ethynyl-6-fluoronaphthalen-2-ol;
5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-11-carbonitrile;
5-(8-ethynyl-7-fluoro-3-hydroxynaphthalen-1-yl)-4-fluoro-2-{1[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-11-carboxamide;
5-chloro-4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-6-fluoronaphthalen-2-ol;
4-(10-benzyl-4-fluoro-2-{[(2R,7aS)-2-fluorotetrahydro-1H-pyrrolizin-7a(5H)-yl]methoxy}-7,8,9,10-tetrahydro-1,3,6,10-tetraazacyclohepta[1,2,3-de]naphthalen-5-yl)-5-ethynyl-6-fluoronaphthalen-2-ol;
(8aS,9S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7-oxa-1,3,6,12a-tetraazabenzo[4,5]cyclohepta[1,2,3-de]naphthalen-9-ol;
(8aS,9S)-5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7-oxa-1,3,6,12a-tetraazabenzo[4,5]cyclohepta[1,2,3-de]naphthalen-9-ol;
4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-5,6-difluoronaphthalen-2-ol;
2-amino-4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-1-benzothiophene-3-carbonitrile;
5-ethyl-6-fluoro-4-(4-fluoro-2-{[(2R,7aS)-2-fluorotetrahydro-1H-pyrrolizin-7a(5H)-yl]methoxy}-7,8,9,10-tetrahydro-1,3,6,10-tetraazacyclohepta[1,2,3-de]naphthalen-5-yl)naphthalen-2-ol;
(8aS)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-11-carbonitrile;
5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol;
(8aS,11S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-11-methyl-8,8a,9,10,11,12-hexahydro-7-oxa-1,3,6,12a-tetraazabenzo[4,5]cyclohepta[1,2,3-de]naphthalen-11-ol;
5-ethynyl-6-fluoro-4-(4-fluoro-2-{[(2R,7aS)-2-fluorotetrahydro-1H-pyrrolizin-7a(5H)-yl]methoxy}-8,8a,9,10,11,12-hexahydro-7H-1,3,6,12a-tetraazabenzo[4,5]cyclohepta[1,2,3-de]naphthalen-5-yl)naphthalen-2-ol;
5-ethynyl-6-fluoro-4-(4-fluoro-2-{[(2R,7aS)-2-fluorotetrahydro-1H-pyrrolizin-7a(5H)-yl]methoxy}-7,8,9,10-tetrahydro-1,3,6,10-tetraazacyclohepta[1,2,3-de]naphthalen-5-yl)naphthalen-2-ol;
5-ethyl-6-fluoro-4-[(9S)-4-fluoro-2-{[(2R,7aS)-2-fluorotetrahydro-1H-pyrrolizin-7a(5H)-yl]methoxy}-9,10-dimethyl-9,10-dihydro-8H-7-oxa-1,3,6,10-tetraazacyclohepta[1,2,3-de]naphthalen-5-yl]naphthalen-2-ol;
5-ethyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-9,10-dihydro-8H-7-oxa-1,3,6,10-tetraazacyclohepta[1,2,3-de]naphthalen-5-yl]naphthalen-2-ol;
1-[8-(10-cyclopropyl-4-fluoro-2-{[(2R,7aS)-2-fluorotetrahydro-1H-pyrrolizin-7a(5H)-yl]methoxy}-9-methyl-9,10-dihydro-8H-7-oxa-1,3,6,10-tetraazacyclohepta[1,2,3-de]naphthalen-5-yl)-2-fluoro-6-hydroxynaphthalen-1-yl]ethan-1-one;
4-(9-cyclopropyl-4-fluoro-2-(((2R,7aS)-2-fluorotetrahydro-1H-pyrrolizin-7a(5H)-yl)methoxy)-10-methyl-9,10-dihydro-8H-7-oxa-1,3,6,10-tetraazacyclohepta[de]naphthalen-5-yl)-5-ethynyl-6-fluoronaphthalen-2-ol;
4-(9-cyclopropyl-4-fluoro-2-(((2R,7aS)-2-fluorotetrahydro-1H-pyrrolizin-7a(5H)-yl)methoxy)-10-methyl-9,10-dihydro-8H-7-oxa-1,3,6,10-tetraazacyclohepta [de]naphthalen-5-yl)-5-ethynyl-6-fluoronaphthalen-2-ol;
5-ethyl-6-fluoro-4-((5aS)-1-fluoro-11-(((2R,7aS)-2-fluorotetrahydro-1H-pyrrolizin-7a(5H)-yl)methoxy)-5-methyl-5,5a,6,7,8,9-hexahydro-4-oxa-3,9a,10,12-tetraazabenzo[4,5]cyclohepta[1,2,3-de]naphthalen-2-yl)naphthalen-2-ol;
4-((S)-10-ethyl-4-fluoro-2-(((2R,7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-5-ethynyl-6-fluoronaphthalen-2-ol;
4-((S)-10-(2,2-difluoroethyl)-4-fluoro-2-(((2R,7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-5-ethynyl-6-fluoronaphthalen-2-ol; 5-ethynyl-6-fluoro-4-(4-fluoro-2-(((2R,7aS)-2-fluorotetrahydro-1H-pyrrolizin-7a(5H)-yl)methoxy)-10-methyl-4′,5′-dihydro-2′H,8H,10H-7-oxa-1,3,6,10-tetraazaspiro[cyclohepta[de]naphthalene-9,3′-furan]-1 (10a),2,3a,3a1 (6a),5-pentaen-5-yl)naphthalen-2-ol;
5-ethynyl-6-fluoro-4-((S)-4-fluoro-2-(((2R,7aS)-2-fluorotetrahydro-1H-pyrrolizin-7a(5H)-yl)methoxy)-10-((R)-2-hydroxypropyl)-9-methyl-9,10-dihydro-8H-7-oxa-1,3,6,10-tetraazacyclohepta[de]naphthalen-5-yl)naphthalen-2-ol;
4-((S)-10-(2,2-difluoroethyl)-4-fluoro-2-(((2R,7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-5-ethyl-6-fluoronaphthalen-2-ol;
5-ethyl-6-fluoro-4-(4-fluoro-2-(((2R,7aS)-2-fluorotetrahydro-1H-pyrrolizin-7a(5H)-yl)methoxy)-10-methyl-4′,5′-dihydro-2′H,8H,10H-7-oxa-1,3,6,10-tetraazaspiro[cyclohepta[de]naphthalene-9,3′-furan]-1 (10a),2,3a,3a1 (6a),5-pentaen-5-yl)naphthalen-2-ol; and
5-ethynyl-6-fluoro-4-((S)-4-fluoro-2-(((2R,7aS)-2-fluorotetrahydro-1H-pyrrolizin-7a(5H)-yl)methoxy)-9-methyl-10-(2,2,2-trifluoroethyl)-9,10-dihydro-8H-7-oxa-1,3,6,10-tetraazacyclohepta[de]naphthalen-5-yl)naphthalen-2-ol;
   or a pharmaceutically acceptable salt thereof.

In some embodiments, the compound is not of the formula

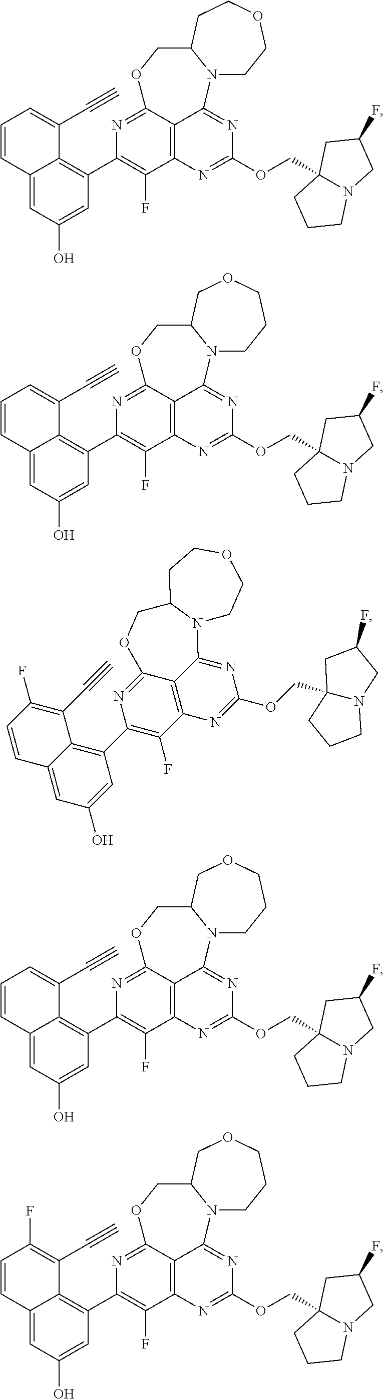 [SMILES: C#Cc1cccc2cc(O)cc(-c3nc4c5c(nc(OC[C@@]67CCCN6C[C@H](F)C7)nc5c3F)N3CCOCCC3CO4)c12 ; C#Cc1cccc2cc(O)cc(-c3nc4c5c(nc(OC[C@@]67CCCN6C[C@H](F)C7)nc5c3F)N3CCCOCC3CO4)c12 ; C#Cc1c(F)ccc2cc(O)cc(-c3nc4c5c(nc(OC[C@@]67CCCN6C[C@H](F)C7)nc5c3F)N3CCOCCC3CO4)c12 ; C#Cc1cccc2cc(O)cc(-c3nc4c5c(nc(OC[C@@]67CCCN6C[C@H](F)C7)nc5c3F)N3CCCOCC3CO4)c12 ; C#Cc1c(F)ccc2cc(O)cc(-c3nc4c5c(nc(OC[C@@]67CCCN6C[C@H](F)C7)nc5c3F)N3CCCOCC3CO4)c12]

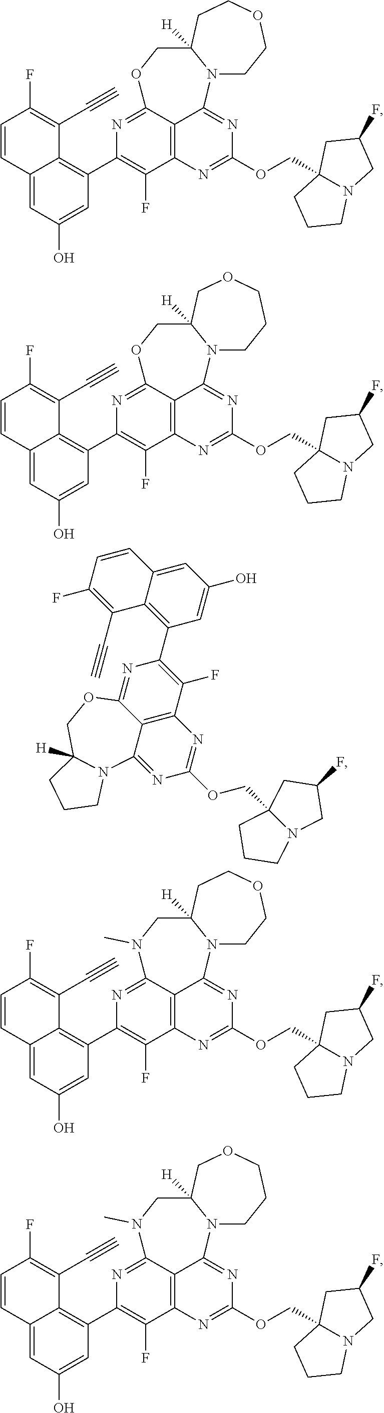

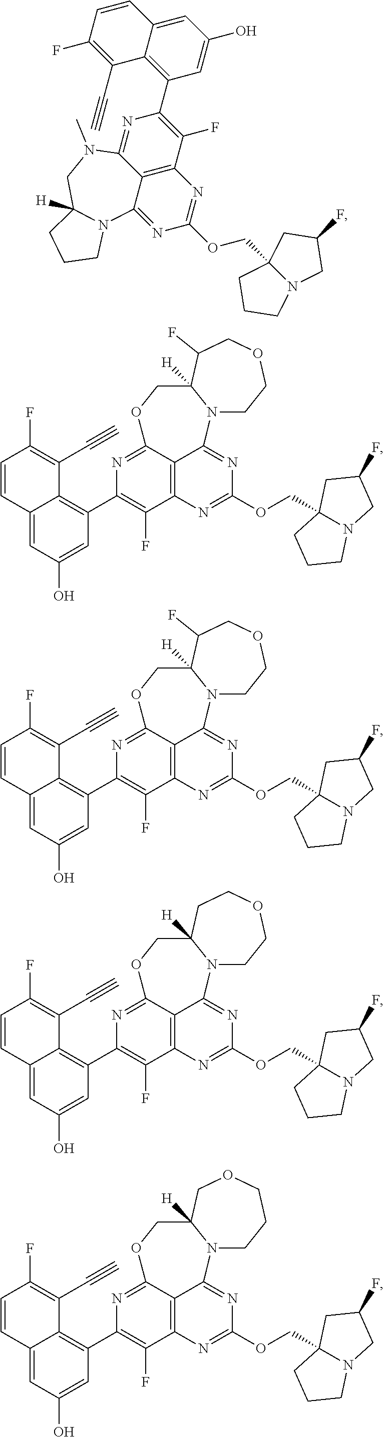

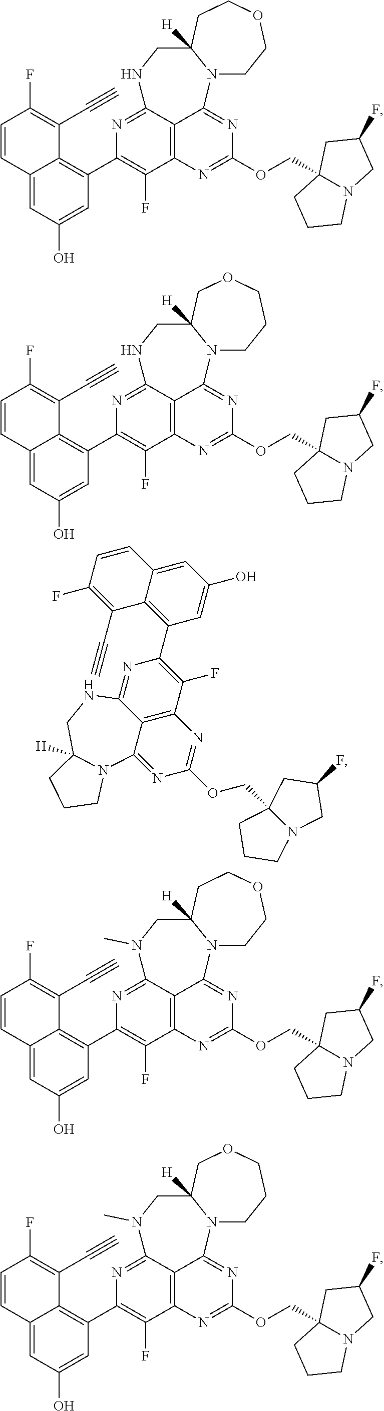

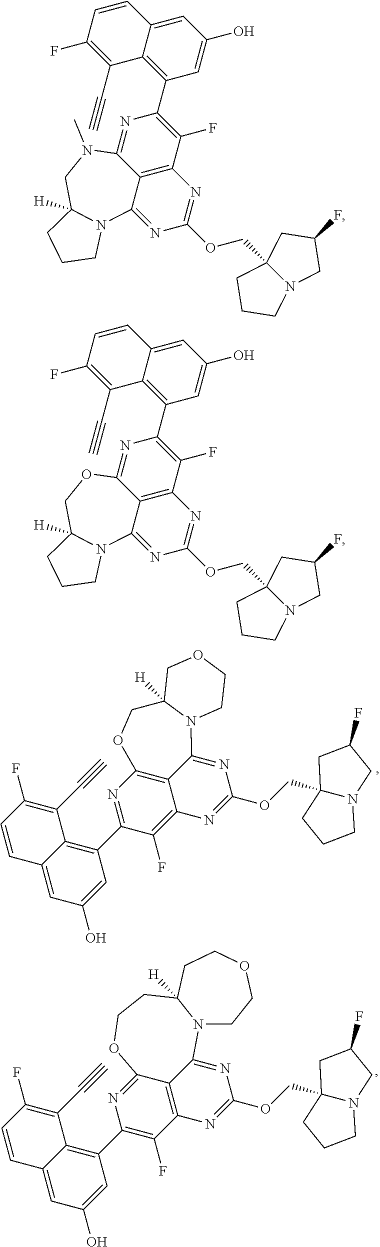

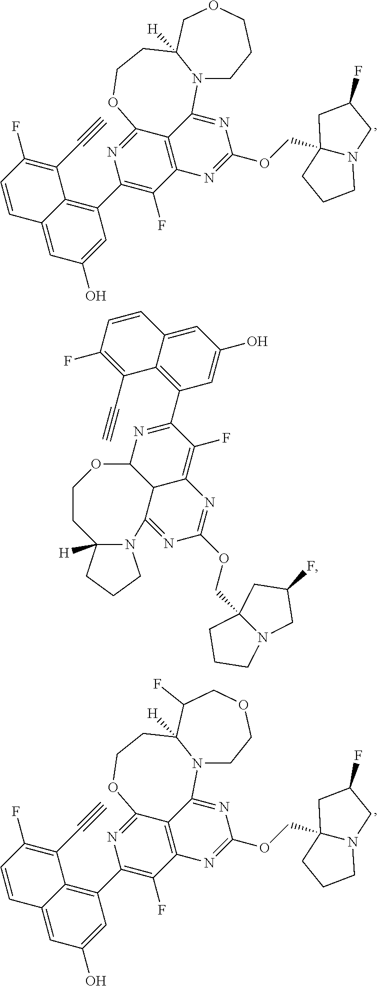

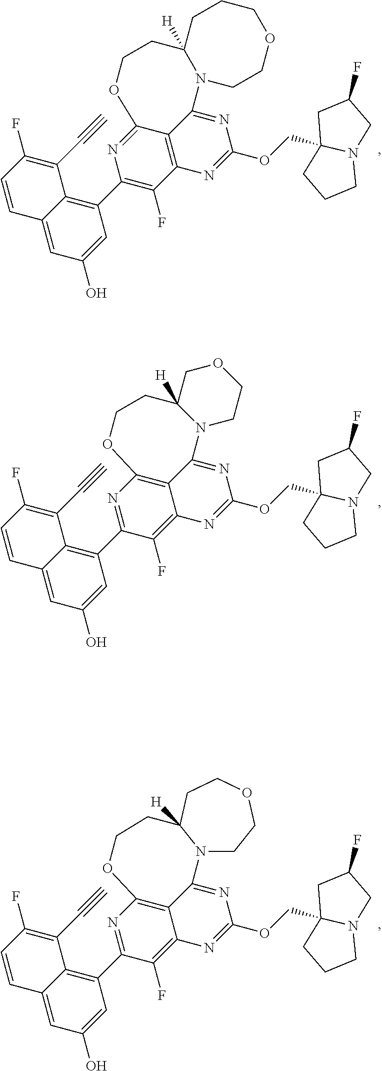 [SMILES: C#Cc1c(F)ccc2cc(O)cc(-c3nc4c5c(nc(OC[C@@]67CCCN6C[C@H](F)C7)nc5c3F)N3CCOCCC[C@@H]3CCO4)c12 ; C#Cc1c(F)ccc2cc(O)cc(-c3nc4c5c(nc(OC[C@@]67CCCN6C[C@H](F)C7)nc5c3F)N3CCOC[C@H]3CCO4)c12 ; C#Cc1c(F)ccc2cc(O)cc(-c3nc4c5c(nc(OC[C@@]67CCCN6C[C@H](F)C7)nc5c3F)N3CCOCC[C@H]3CCO4)c12]

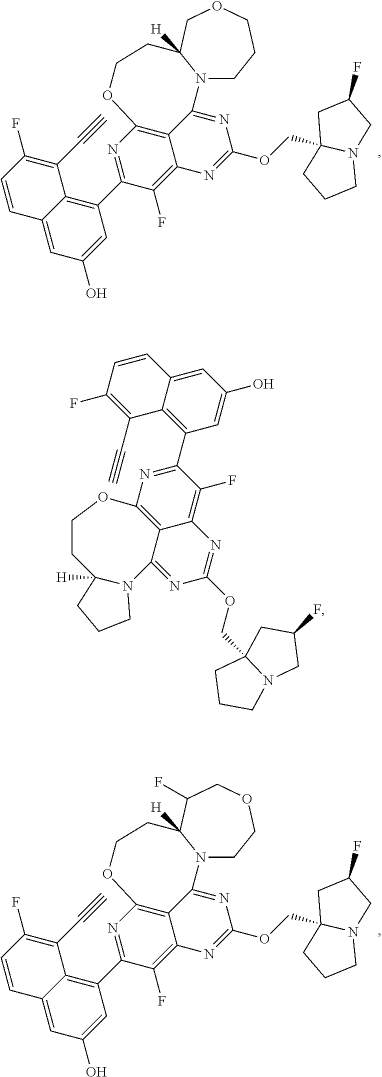 [SMILES: C#Cc1c(F)ccc2cc(O)cc(-c3nc4c5c(nc(OC[C@@]67CCCN6C[C@H](F)C7)nc5c3F)N3CCCOC[C@H]3CCO4)c12 ; C#Cc1c(F)ccc2cc(O)cc(-c3nc4c5c(nc(OC[C@@]67CCCN6C[C@H](F)C7)nc5c3F)N3CCC[C@H]3CCO4)c12 ; C#Cc1c(F)ccc2cc(O)cc(-c3nc4c5c(nc(OC[C@@]67CCCN6C[C@H](F)C7)nc5c3F)N3CCOCC(F)[C@H]3CCO4)c12]

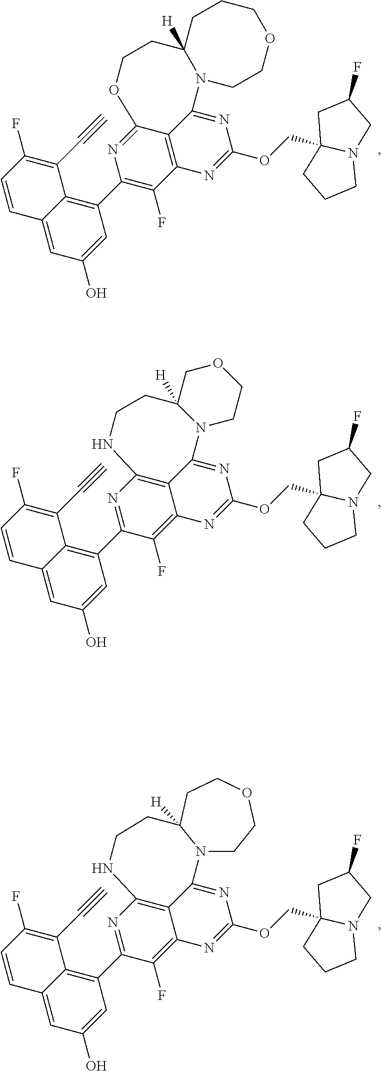 [SMILES: C#Cc1c(F)ccc2cc(O)cc(-c3nc4c5c(nc(OC[C@@]67CCCN6C[C@H](F)C7)nc5c3F)N3CCOCCC[C@H]3CCO4)c12 ; C#Cc1c(F)ccc2cc(O)cc(-c3nc4c5c(nc(OC[C@@]67CCCN6C[C@H](F)C7)nc5c3F)N3CCOC[C@@H]3CCN4)c12 ; C#Cc1c(F)ccc2cc(O)cc(-c3nc4c5c(nc(OC[C@@]67CCCN6C[C@H](F)C7)nc5c3F)N3CCOCC[C@@H]3CCN4)c12]

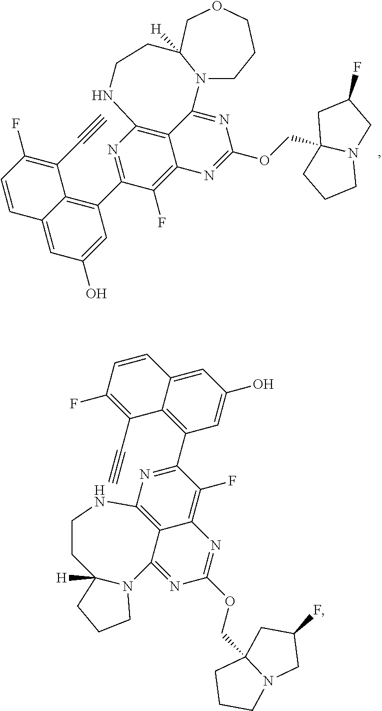 [SMILES: C#Cc1c(F)ccc2cc(O)cc(-c3nc4c5c(nc(OC[C@@]67CCCN6C[C@H](F)C7)nc5c3F)N3CCCOC[C@@H]3CCN4)c12 ; C#Cc1c(F)ccc2cc(O)cc(-c3nc4c5c(nc(OC[C@@]67CCCN6C[C@H](F)C7)nc5c3F)N3CCC[C@@H]3CCN4)c12]

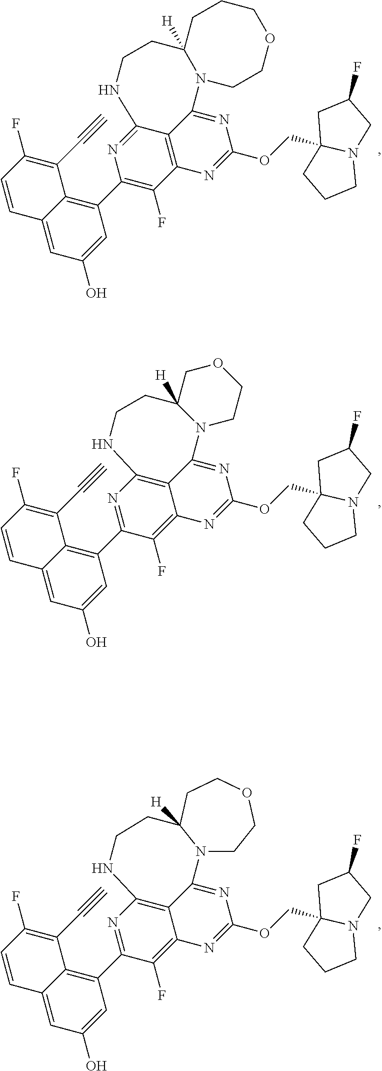 [SMILES: C#Cc1c(F)ccc2cc(O)cc(-c3nc4c5c(nc(OC[C@@]67CCCN6C[C@H](F)C7)nc5c3F)N3CCOCCC[C@@H]3CCN4)c12 ; C#Cc1c(F)ccc2cc(O)cc(-c3nc4c5c(nc(OC[C@@]67CCCN6C[C@H](F)C7)nc5c3F)N3CCOC[C@H]3CCN4)c12 ; C#Cc1c(F)ccc2cc(O)cc(-c3nc4c5c(nc(OC[C@@]67CCCN6C[C@H](F)C7)nc5c3F)N3CCOCC[C@H]3CCN4)c12]

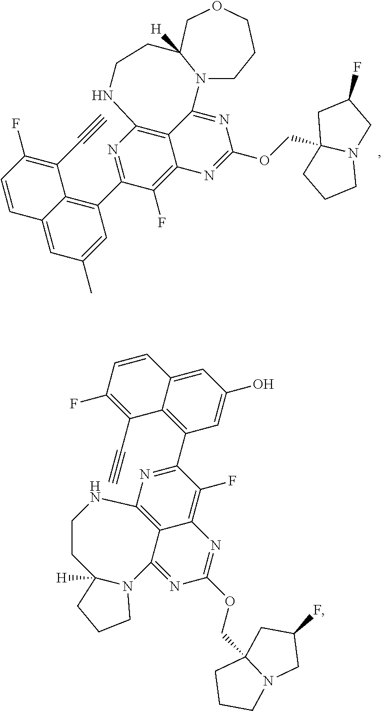 [SMILES: C#Cc1c(F)ccc2cc(C)cc(-c3nc4c5c(nc(OC[C@@]67CCCN6C[C@H](F)C7)nc5c3F)N3CCCOC[C@H]3CCN4)c12 ; C#Cc1c(F)ccc2cc(O)cc(-c3nc4c5c(nc(OC[C@@]67CCCN6C[C@H](F)C7)nc5c3F)N3CCC[C@H]3CCN4)c12]

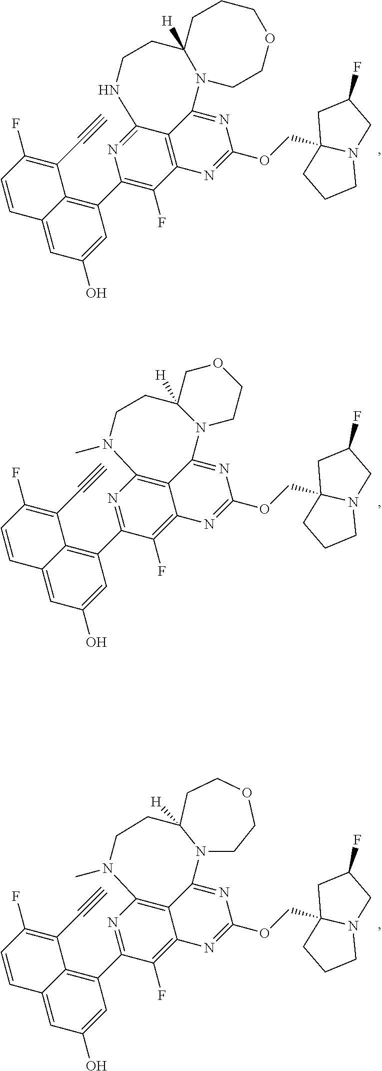 [SMILES: C#Cc1c(F)ccc2cc(O)cc(-c3nc4c5c(nc(OC[C@@]67CCCN6C[C@H](F)C7)nc5c3F)N3CCOCCC[C@H]3CCN4)c12 ; C#Cc1c(F)ccc2cc(O)cc(-c3nc4c5c(nc(OC[C@@]67CCCN6C[C@H](F)C7)nc5c3F)N3CCOC[C@@H]3CCN4C)c12 ; C#Cc1c(F)ccc2cc(O)cc(-c3nc4c5c(nc(OC[C@@]67CCCN6C[C@H](F)C7)nc5c3F)N3CCOCC[C@@H]3CCN4C)c12]

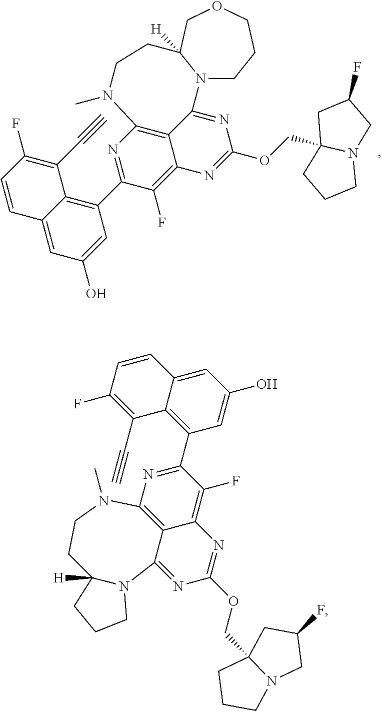 [SMILES: C#Cc1c(F)ccc2cc(O)cc(-c3nc4c5c(nc(OC[C@@]67CCCN6C[C@H](F)C7)nc5c3F)N3CCCOC[C@@H]3CCN4C)c12 ; C#Cc1c(F)ccc2cc(O)cc(-c3nc4c5c(nc(OC[C@@]67CCCN6C[C@H](F)C7)nc5c3F)N3CCC[C@@H]3CCN4C)c12]

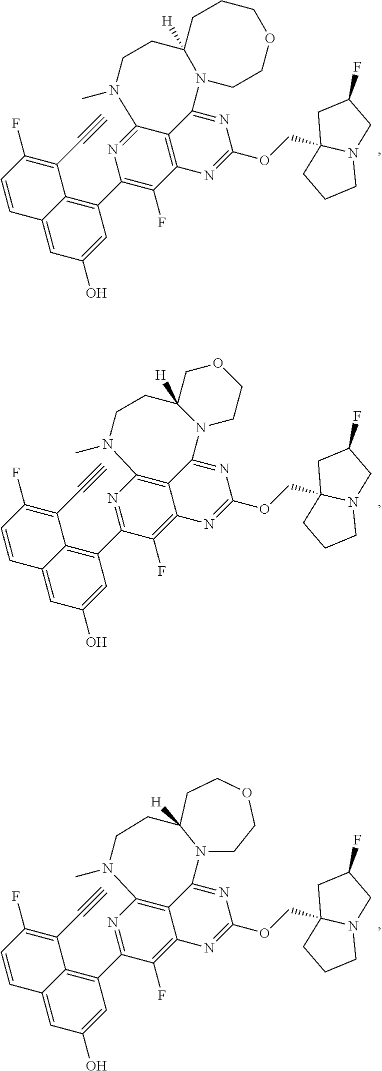 [SMILES: C#Cc1c(F)ccc2cc(O)cc(-c3nc4c5c(nc(OC[C@@]67CCCN6C[C@H](F)C7)nc5c3F)N3CCOCCC[C@@H]3CCN4C)c12 ; C#Cc1c(F)ccc2cc(O)cc(-c3nc4c5c(nc(OC[C@@]67CCCN6C[C@H](F)C7)nc5c3F)N3CCOC[C@H]3CCN4C)c12 ; C#Cc1c(F)ccc2cc(O)cc(-c3nc4c5c(nc(OC[C@@]67CCCN6C[C@H](F)C7)nc5c3F)N3CCOCC[C@H]3CCN4C)c12]

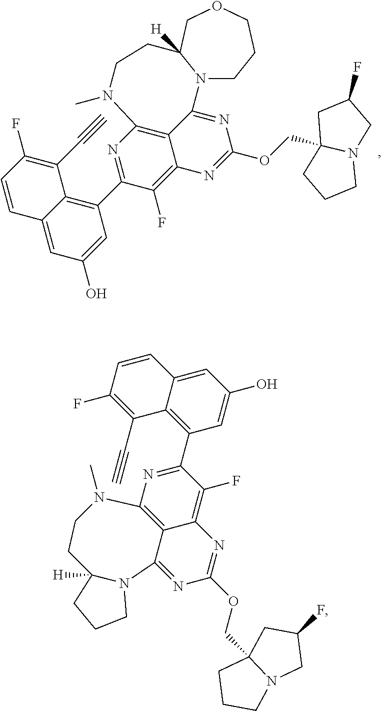 [SMILES: C#Cc1c(F)ccc2cc(O)cc(-c3nc4c5c(nc(OC[C@@]67CCCN6C[C@H](F)C7)nc5c3F)N3CCCOC[C@H]3CCN4C)c12 ; C#Cc1c(F)ccc2cc(O)cc(-c3nc4c5c(nc(OC[C@@]67CCCN6C[C@H](F)C7)nc5c3F)N3CCC[C@H]3CCN4C)c12]

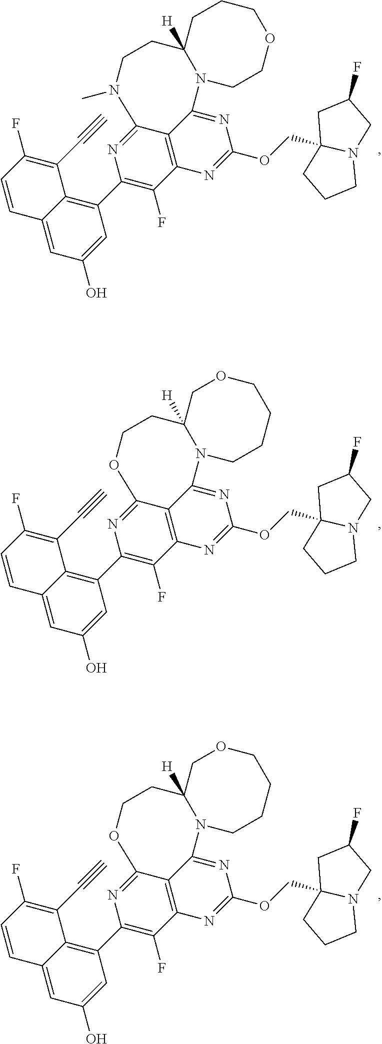 [SMILES: C#Cc1c(F)ccc2cc(O)cc(-c3nc4c5c(nc(OC[C@@]67CCCN6C[C@H](F)C7)nc5c3F)N3CCOCCC[C@H]3CCN4C)c12 ; C#Cc1c(F)ccc2cc(O)cc(-c3nc4c5c(nc(OC[C@@]67CCCN6C[C@H](F)C7)nc5c3F)N3CCCCOC[C@@H]3CCO4)c12 ; C#Cc1c(F)ccc2cc(O)cc(-c3nc4c5c(nc(OC[C@@]67CCCN6C[C@H](F)C7)nc5c3F)N3CCCCOC[C@H]3CCO4)c12]

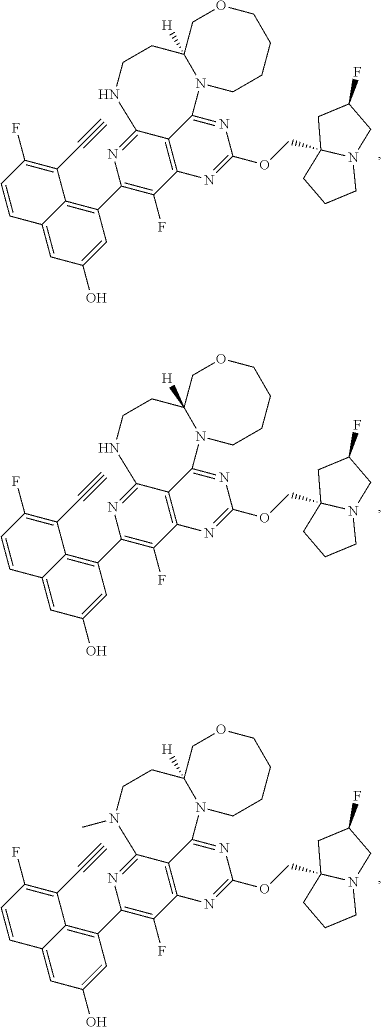 [SMILES: C#Cc1c(F)ccc2cc(O)cc(-c3nc4c5c(nc(OC[C@@]67CCCN6C[C@H](F)C7)nc5c3F)N3CCCCOC[C@@H]3CCN4)c12 ; C#Cc1c(F)ccc2cc(O)cc(-c3nc4c5c(nc(OC[C@@]67CCCN6C[C@H](F)C7)nc5c3F)N3CCCCOC[C@H]3CCN4)c12 ; C#Cc1c(F)ccc2cc(O)cc(-c3nc4c5c(nc(OC[C@@]67CCCN6C[C@H](F)C7)nc5c3F)N3CCCCOC[C@@H]3CCN4C)c12]

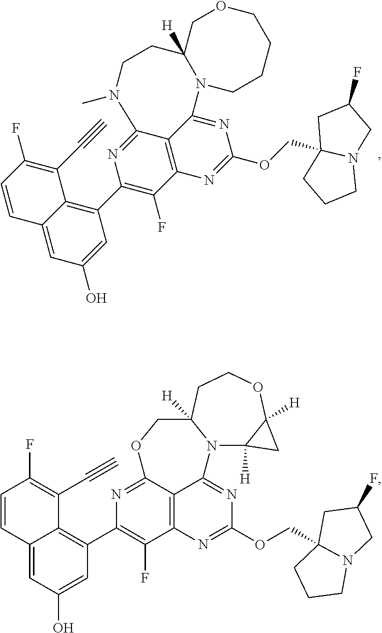 [SMILES: C#Cc1c(F)ccc2cc(O)cc(-c3nc4c5c(nc(OC[C@@]67CCCN6C[C@H](F)C7)nc5c3F)N3CCCCOC[C@H]3CCN4C)c12 ; C#Cc1c(F)ccc2cc(O)cc(-c3nc4c5c(nc(OC[C@@]67CCCN6C[C@H](F)C7)nc5c3F)N3[C@H](CCO[C@H]5C[C@H]53)CO4)c12]

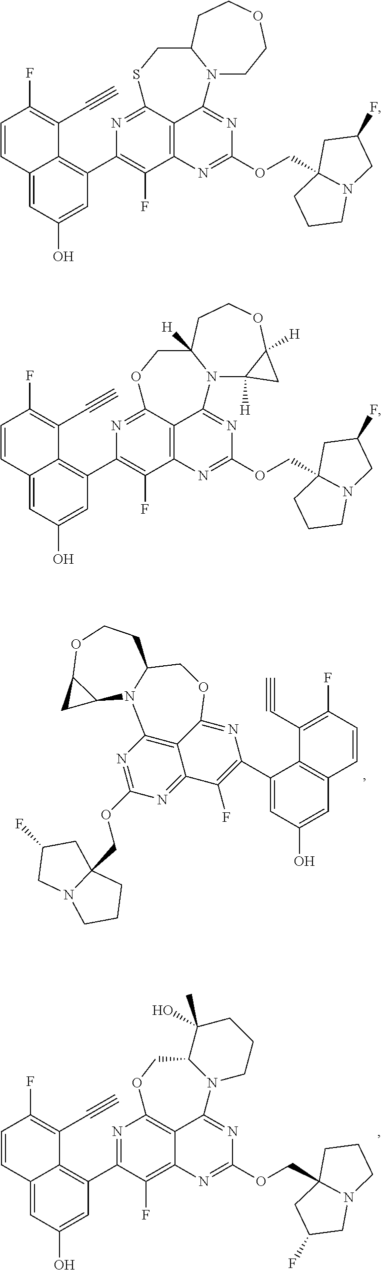 [SMILES: C#Cc1c(F)ccc2cc(O)cc(-c3nc4c5c(nc(OC[C@@]67CCCN6C[C@H](F)C7)nc5c3F)N3CCOCCC3CS4)c12 ; C#Cc1c(F)ccc2cc(O)cc(-c3nc4c5c(nc(OC[C@@]67CCCN6C[C@H](F)C7)nc5c3F)N3[C@@H](CCO[C@H]5C[C@H]53)CO4)c12 ; C#Cc1c(F)ccc2cc(O)cc(-c3nc4c5c(nc(OC[C@@]67CCCN6C[C@H](F)C7)nc5c3F)N3[C@@H](CCO[C@@H]5C[C@@H]53)CO4)c12 ; C#Cc1c(F)ccc2cc(O)cc(-c3nc4c5c(nc(OC[C@@]67CCCN6C[C@H](F)C7)nc5c3F)N3CCC[C@@](C)(O)[C@H]3CO4)c12]

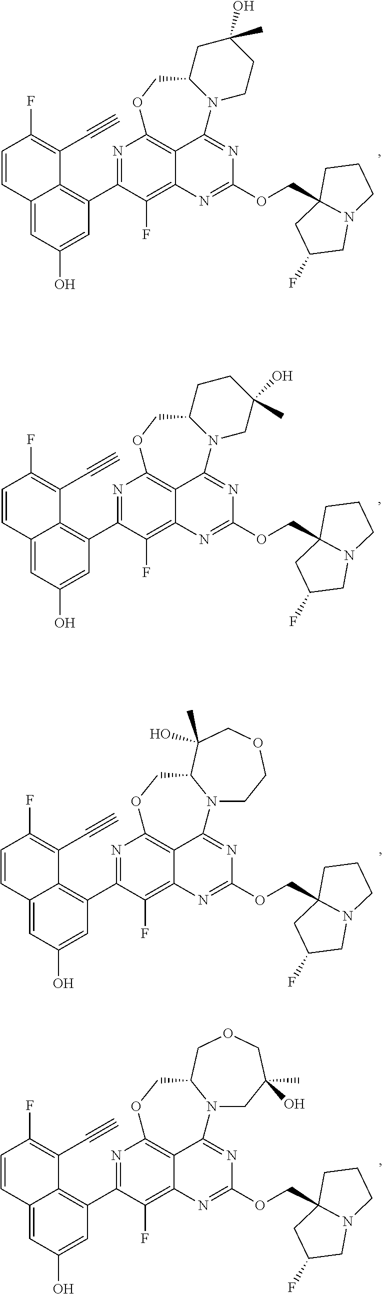 [SMILES: C#Cc1c(F)ccc2cc(O)cc(-c3nc4c5c(nc(OC[C@@]67CCCN6C[C@H](F)C7)nc5c3F)N3CC[C@@](C)(O)C[C@H]3CO4)c12 ; C#Cc1c(F)ccc2cc(O)cc(-c3nc4c5c(nc(OC[C@@]67CCCN6C[C@H](F)C7)nc5c3F)N3C[C@@](C)(O)CC[C@H]3CO4)c12 ; C#Cc1c(F)ccc2cc(O)cc(-c3nc4c5c(nc(OC[C@@]67CCCN6C[C@H](F)C7)nc5c3F)N3CCOC[C@@](C)(O)[C@H]3CO4)c12 ; C#Cc1c(F)ccc2cc(O)cc(-c3nc4c5c(nc(OC[C@@]67CCCN6C[C@H](F)C7)nc5c3F)N3C[C@](C)(O)COC[C@H]3CO4)c12]

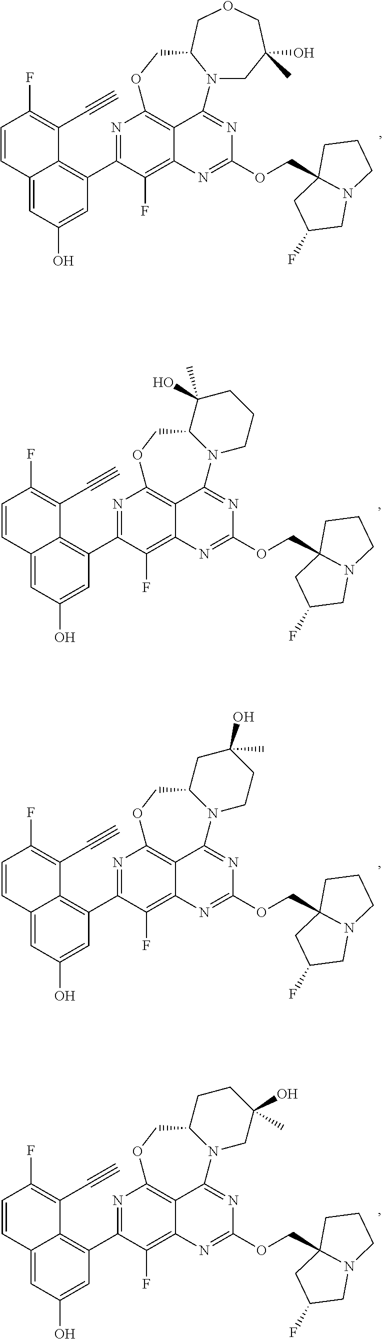 [SMILES: C#Cc1c(F)ccc2cc(O)cc(-c3nc4c5c(nc(OC[C@@]67CCCN6C[C@H](F)C7)nc5c3F)N3C[C@@](C)(O)COC[C@H]3CO4)c12 ; C#Cc1c(F)ccc2cc(O)cc(-c3nc4c5c(nc(OC[C@@]67CCCN6C[C@H](F)C7)nc5c3F)N3CCC[C@](C)(O)[C@H]3CO4)c12 ; C#Cc1c(F)ccc2cc(O)cc(-c3nc4c5c(nc(OC[C@@]67CCCN6C[C@H](F)C7)nc5c3F)N3CC[C@](C)(O)C[C@H]3CO4)c12 ; C#Cc1c(F)ccc2cc(O)cc(-c3nc4c5c(nc(OC[C@@]67CCCN6C[C@H](F)C7)nc5c3F)N3C[C@](C)(O)CC[C@H]3CO4)c12]

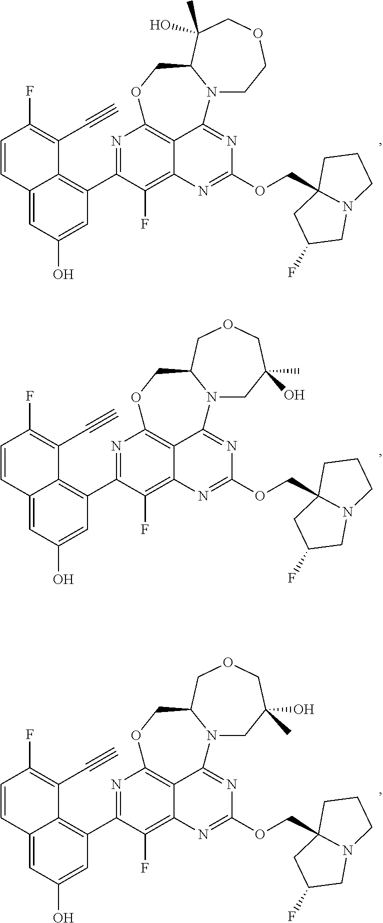 [SMILES: C#Cc1c(F)ccc2cc(O)cc(-c3nc4c5c(nc(OC[C@@]67CCCN6C[C@H](F)C7)nc5c3F)N3CCOC[C@@](C)(O)[C@@H]3CO4)c12 ; C#Cc1c(F)ccc2cc(O)cc(-c3nc4c5c(nc(OC[C@@]67CCCN6C[C@H](F)C7)nc5c3F)N3C[C@](C)(O)COC[C@@H]3CO4)c12 ; C#Cc1c(F)ccc2cc(O)cc(-c3nc4c5c(nc(OC[C@@]67CCCN6C[C@H](F)C7)nc5c3F)N3C[C@@](C)(O)COC[C@@H]3CO4)c12]

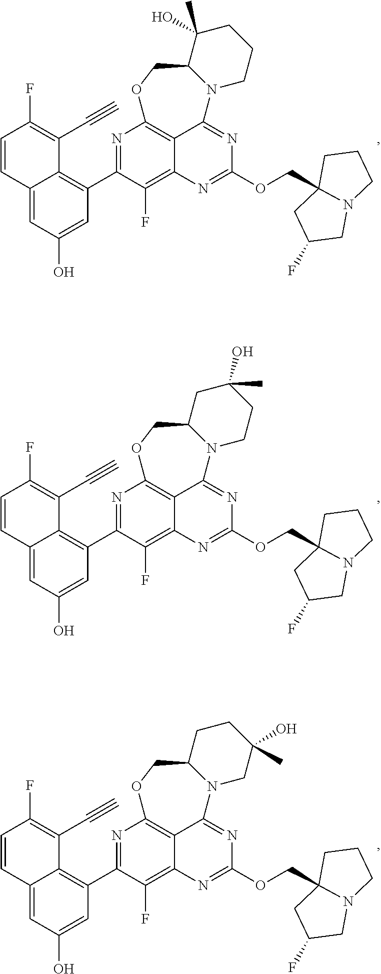 [SMILES: C#Cc1c(F)ccc2cc(O)cc(-c3nc4c5c(nc(OC[C@@]67CCCN6C[C@H](F)C7)nc5c3F)N3CCC[C@@](C)(O)[C@@H]3CO4)c12 ; C#Cc1c(F)ccc2cc(O)cc(-c3nc4c5c(nc(OC[C@@]67CCCN6C[C@H](F)C7)nc5c3F)N3CC[C@@](C)(O)C[C@@H]3CO4)c12 ; C#Cc1c(F)ccc2cc(O)cc(-c3nc4c5c(nc(OC[C@@]67CCCN6C[C@H](F)C7)nc5c3F)N3C[C@@](C)(O)CC[C@@H]3CO4)c12]

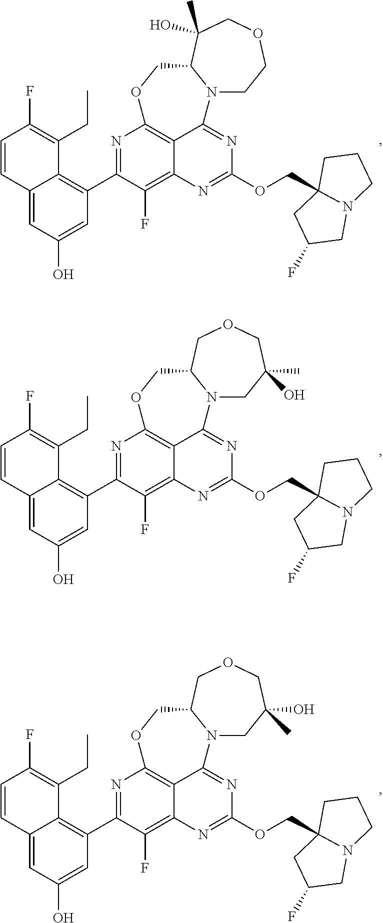 [SMILES: CCc1c(F)ccc2cc(O)cc(-c3nc4c5c(nc(OC[C@@]67CCCN6C[C@H](F)C7)nc5c3F)N3CCOC[C@@](C)(O)[C@H]3CO4)c12 ; CCc1c(F)ccc2cc(O)cc(-c3nc4c5c(nc(OC[C@@]67CCCN6C[C@H](F)C7)nc5c3F)N3C[C@](C)(O)COC[C@H]3CO4)c12 ; CCc1c(F)ccc2cc(O)cc(-c3nc4c5c(nc(OC[C@@]67CCCN6C[C@H](F)C7)nc5c3F)N3C[C@@](C)(O)COC[C@H]3CO4)c12]

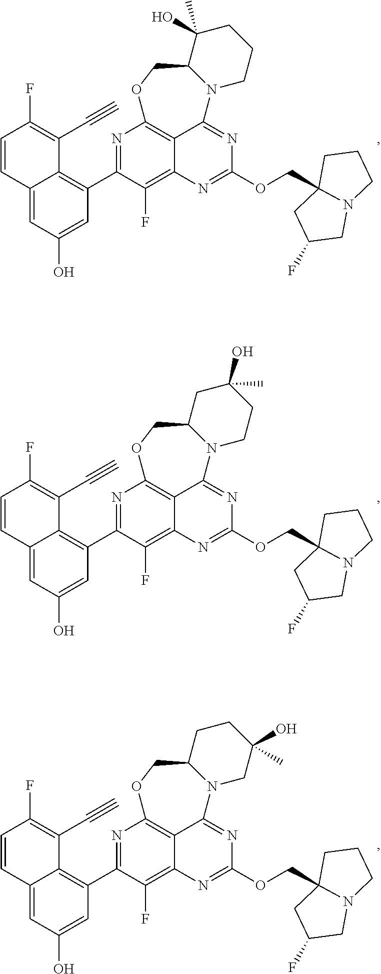 [SMILES: C#Cc1c(F)ccc2cc(O)cc(-c3nc4c5c(nc(OC[C@@]67CCCN6C[C@H](F)C7)nc5c3F)N3CCC[C@](C)(O)[C@@H]3CO4)c12 ; C#Cc1c(F)ccc2cc(O)cc(-c3nc4c5c(nc(OC[C@@]67CCCN6C[C@H](F)C7)nc5c3F)N3CC[C@](C)(O)C[C@@H]3CO4)c12 ; C#Cc1c(F)ccc2cc(O)cc(-c3nc4c5c(nc(OC[C@@]67CCCN6C[C@H](F)C7)nc5c3F)N3C[C@](C)(O)CC[C@@H]3CO4)c12]

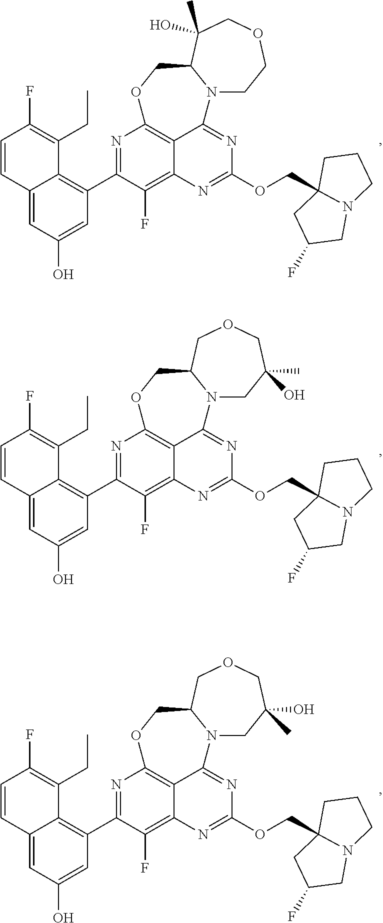 [SMILES: CCc1c(F)ccc2cc(O)cc(-c3nc4c5c(nc(OC[C@@]67CCCN6C[C@H](F)C7)nc5c3F)N3CCOC[C@@](C)(O)[C@@H]3CO4)c12 ; CCc1c(F)ccc2cc(O)cc(-c3nc4c5c(nc(OC[C@@]67CCCN6C[C@H](F)C7)nc5c3F)N3C[C@](C)(O)COC[C@@H]3CO4)c12 ; CCc1c(F)ccc2cc(O)cc(-c3nc4c5c(nc(OC[C@@]67CCCN6C[C@H](F)C7)nc5c3F)N3C[C@@](C)(O)COC[C@@H]3CO4)c12]

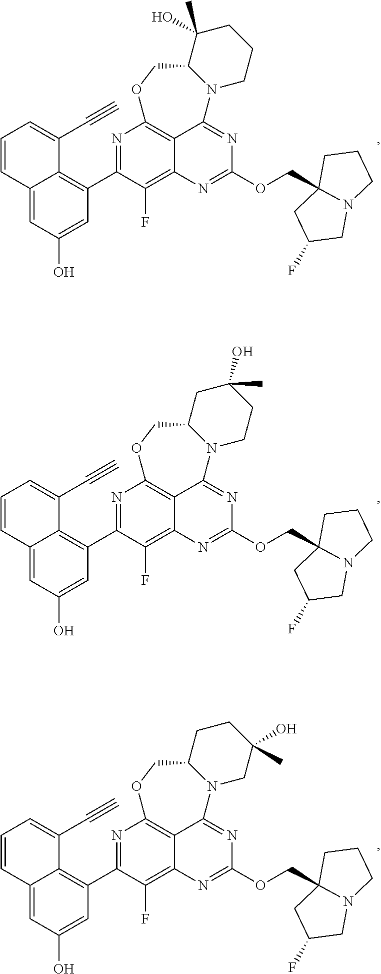 [SMILES: C#Cc1cccc2cc(O)cc(-c3nc4c5c(nc(OC[C@@]67CCCN6C[C@H](F)C7)nc5c3F)N3CCC[C@@](C)(O)[C@H]3CO4)c12 ; C#Cc1cccc2cc(O)cc(-c3nc4c5c(nc(OC[C@@]67CCCN6C[C@H](F)C7)nc5c3F)N3CC[C@@](C)(O)C[C@H]3CO4)c12 ; C#Cc1cccc2cc(O)cc(-c3nc4c5c(nc(OC[C@@]67CCCN6C[C@H](F)C7)nc5c3F)N3C[C@@](C)(O)CC[C@H]3CO4)c12]

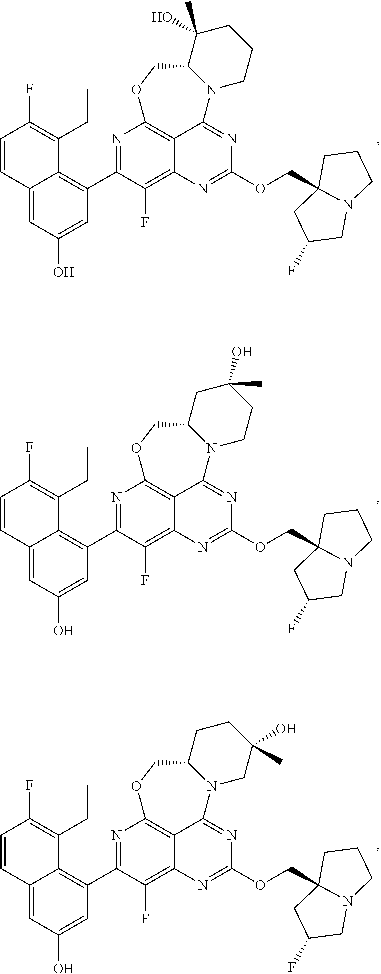 [SMILES: CCc1c(F)ccc2cc(O)cc(-c3nc4c5c(nc(OC[C@@]67CCCN6C[C@H](F)C7)nc5c3F)N3CCC[C@@](C)(O)[C@H]3CO4)c12 ; CCc1c(F)ccc2cc(O)cc(-c3nc4c5c(nc(OC[C@@]67CCCN6C[C@H](F)C7)nc5c3F)N3CC[C@@](C)(O)C[C@H]3CO4)c12 ; CCc1c(F)ccc2cc(O)cc(-c3nc4c5c(nc(OC[C@@]67CCCN6C[C@H](F)C7)nc5c3F)N3C[C@@](C)(O)CC[C@H]3CO4)c12]

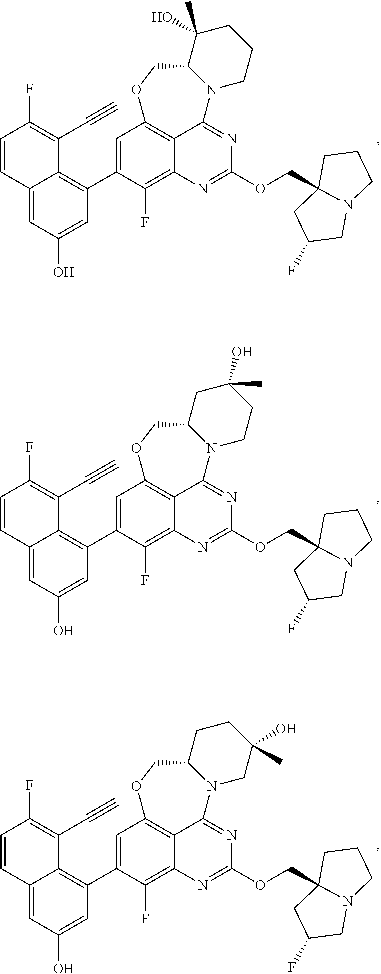 [SMILES: C#Cc1c(F)ccc2cc(O)cc(-c3cc4c5c(nc(OC[C@@]67CCCN6C[C@H](F)C7)nc5c3F)N3CCC[C@@](C)(O)[C@H]3CO4)c12 ; C#Cc1c(F)ccc2cc(O)cc(-c3cc4c5c(nc(OC[C@@]67CCCN6C[C@H](F)C7)nc5c3F)N3CC[C@@](C)(O)C[C@H]3CO4)c12 ; C#Cc1c(F)ccc2cc(O)cc(-c3cc4c5c(nc(OC[C@@]67CCCN6C[C@H](F)C7)nc5c3F)N3C[C@@](C)(O)CC[C@H]3CO4)c12]

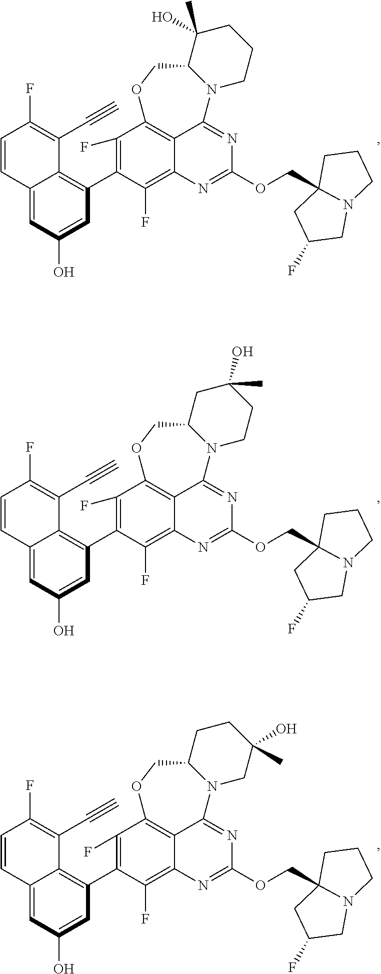

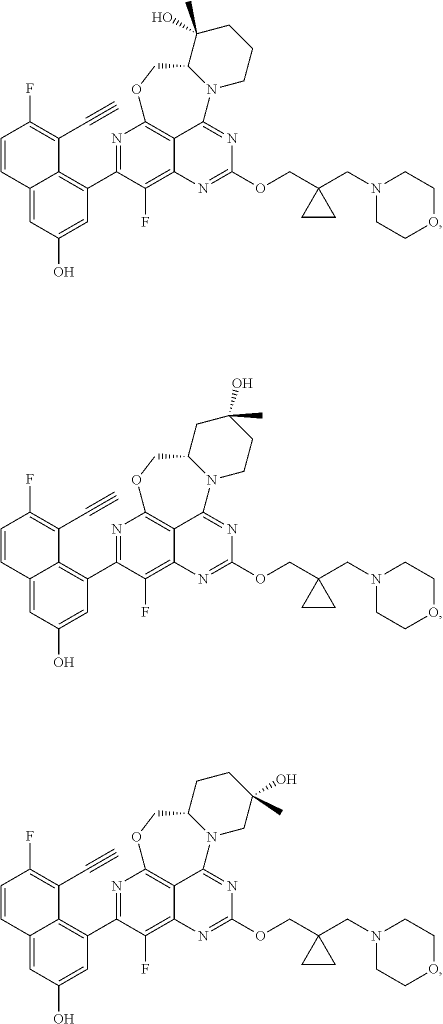 [SMILES: C#Cc1c(F)ccc2cc(O)cc(-c3nc4c5c(nc(OCC6(CN7CCOCC7)CC6)nc5c3F)N3CCC[C@@](C)(O)[C@H]3CO4)c12 ; C#Cc1c(F)ccc2cc(O)cc(-c3nc4c5c(nc(OCC6(CN7CCOCC7)CC6)nc5c3F)N3CC[C@@](C)(O)C[C@H]3CO4)c12 ; C#Cc1c(F)ccc2cc(O)cc(-c3nc4c5c(nc(OCC6(CN7CCOCC7)CC6)nc5c3F)N3C[C@@](C)(O)CC[C@H]3CO4)c12]

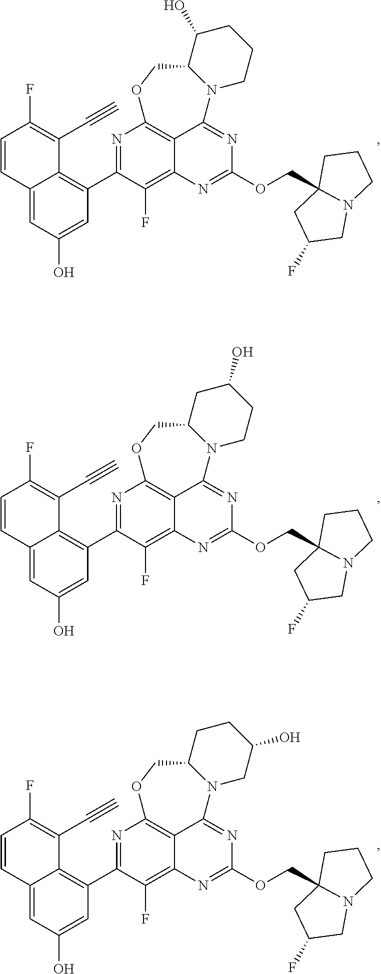 [SMILES: C#Cc1c(F)ccc2cc(O)cc(-c3nc4c5c(nc(OC[C@@]67CCCN6C[C@H](F)C7)nc5c3F)N3CCC[C@@H](O)[C@H]3CO4)c12 ; C#Cc1c(F)ccc2cc(O)cc(-c3nc4c5c(nc(OC[C@@]67CCCN6C[C@H](F)C7)nc5c3F)N3CC[C@@H](O)C[C@H]3CO4)c12 ; C#Cc1c(F)ccc2cc(O)cc(-c3nc4c5c(nc(OC[C@@]67CCCN6C[C@H](F)C7)nc5c3F)N3C[C@@H](O)CC[C@H]3CO4)c12]

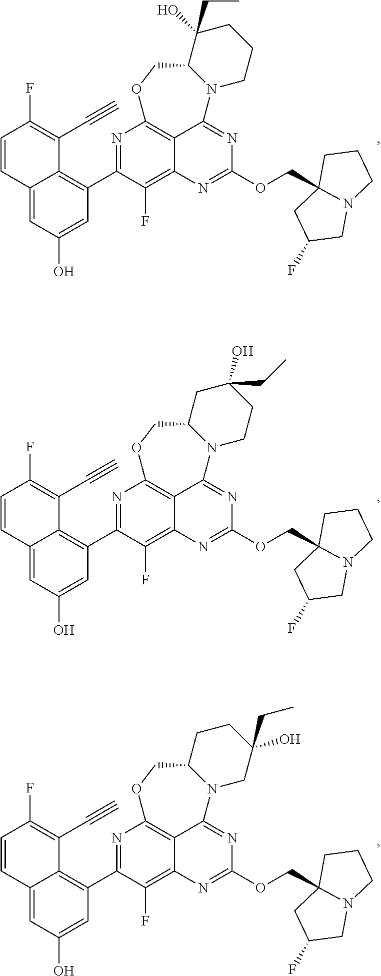 [SMILES: C#Cc1c(F)ccc2cc(O)cc(-c3nc4c5c(nc(OC[C@@]67CCCN6C[C@H](F)C7)nc5c3F)N3CCC[C@](O)(CC)[C@H]3CO4)c12 ; C#Cc1c(F)ccc2cc(O)cc(-c3nc4c5c(nc(OC[C@@]67CCCN6C[C@H](F)C7)nc5c3F)N3CC[C@](O)(CC)C[C@H]3CO4)c12 ; C#Cc1c(F)ccc2cc(O)cc(-c3nc4c5c(nc(OC[C@@]67CCCN6C[C@H](F)C7)nc5c3F)N3C[C@](O)(CC)CC[C@H]3CO4)c12]

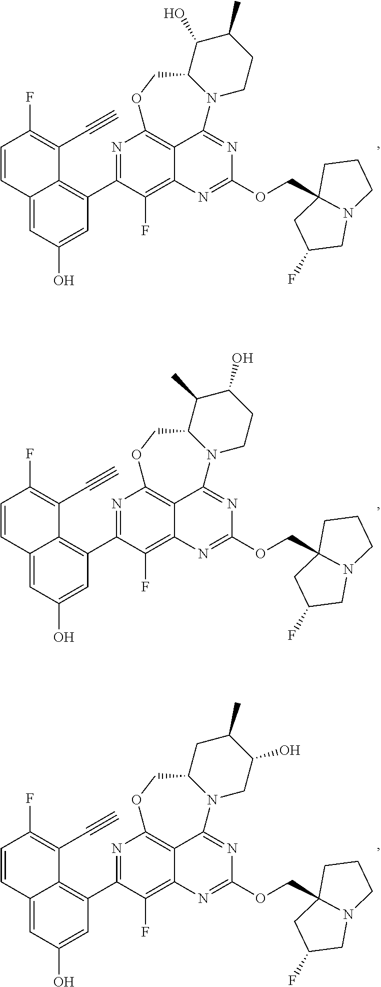 [SMILES: C#Cc1c(F)ccc2cc(O)cc(-c3nc4c5c(nc(OC[C@@]67CCCN6C[C@H](F)C7)nc5c3F)N3CC[C@H](C)[C@@H](O)[C@H]3CO4)c12 ; C#Cc1c(F)ccc2cc(O)cc(-c3nc4c5c(nc(OC[C@@]67CCCN6C[C@H](F)C7)nc5c3F)N3CC[C@@H](O)[C@H](C)[C@H]3CO4)c12 ; C#Cc1c(F)ccc2cc(O)cc(-c3nc4c5c(nc(OC[C@@]67CCCN6C[C@H](F)C7)nc5c3F)N3C[C@@H](O)[C@H](C)C[C@H]3CO4)c12]

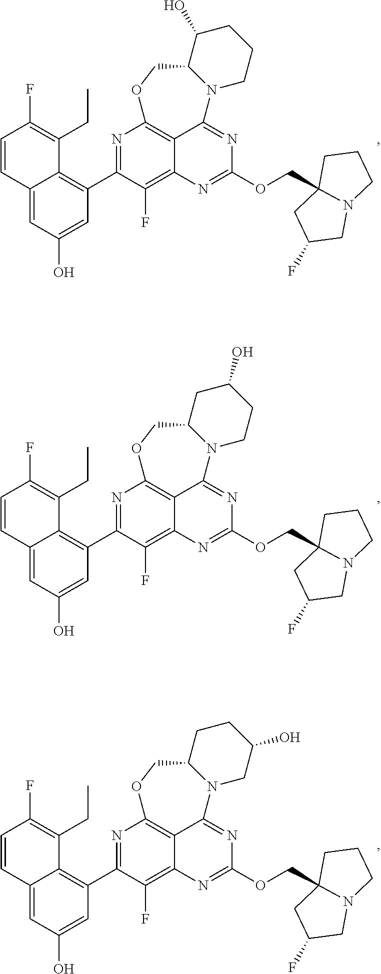 [SMILES: CCc1c(F)ccc2cc(O)cc(-c3nc4c5c(nc(OC[C@@]67CCCN6C[C@H](F)C7)nc5c3F)N3CCC[C@@H](O)[C@H]3CO4)c12 ; CCc1c(F)ccc2cc(O)cc(-c3nc4c5c(nc(OC[C@@]67CCCN6C[C@H](F)C7)nc5c3F)N3CC[C@@H](O)C[C@H]3CO4)c12 ; CCc1c(F)ccc2cc(O)cc(-c3nc4c5c(nc(OC[C@@]67CCCN6C[C@H](F)C7)nc5c3F)N3C[C@@H](O)CC[C@H]3CO4)c12]

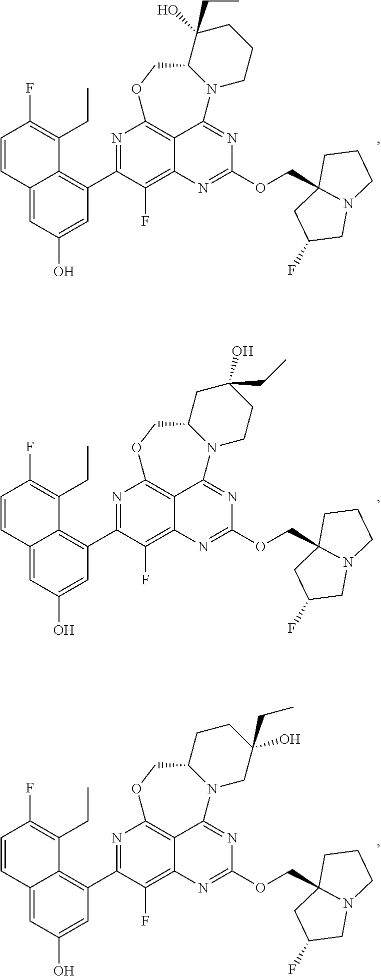 [SMILES: CCc1c(F)ccc2cc(O)cc(-c3nc4c5c(nc(OC[C@@]67CCCN6C[C@H](F)C7)nc5c3F)N3CCC[C@](O)(CC)[C@H]3CO4)c12 ; CCc1c(F)ccc2cc(O)cc(-c3nc4c5c(nc(OC[C@@]67CCCN6C[C@H](F)C7)nc5c3F)N3CC[C@](O)(CC)C[C@H]3CO4)c12 ; CCc1c(F)ccc2cc(O)cc(-c3nc4c5c(nc(OC[C@@]67CCCN6C[C@H](F)C7)nc5c3F)N3C[C@](O)(CC)CC[C@H]3CO4)c12]

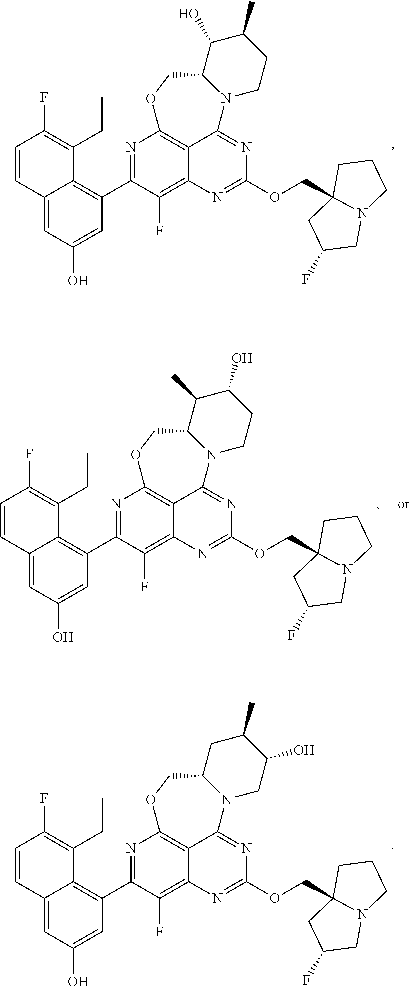 [SMILES: CCc1c(F)ccc2cc(O)cc(-c3nc4c5c(nc(OC[C@@]67CCCN6C[C@H](F)C7)nc5c3F)N3CC[C@H](C)[C@@H](O)[C@H]3CO4)c12 ; CCc1c(F)ccc2cc(O)cc(-c3nc4c5c(nc(OC[C@@]67CCCN6C[C@H](F)C7)nc5c3F)N3CC[C@@H](O)[C@H](C)[C@H]3CO4)c12 ; CCc1c(F)ccc2cc(O)cc(-c3nc4c5c(nc(OC[C@@]67CCCN6C[C@H](F)C7)nc5c3F)N3C[C@@H](O)[C@H](C)C[C@H]3CO4)c12]

The following represent illustrative embodiments of compounds of Formula (I):

| Ex. # | Structure | Name |
|---|---|---|
| 1 | 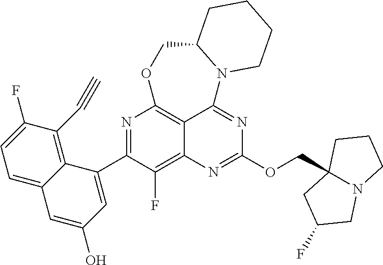 [SMILES: C#Cc1c(F)ccc2cc(O)cc(-c3nc4c5c(nc(OC[C@@]67CCCN6C[C@H](F)C7)nc5c3F)N3CCCC[C@H]3CO4)c12] | 5-ethynyl-6-fluoro-4-[(8aS)-4-fluoro- 2-{[2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-5-yl]naphthalen-2-ol |
| 2 | 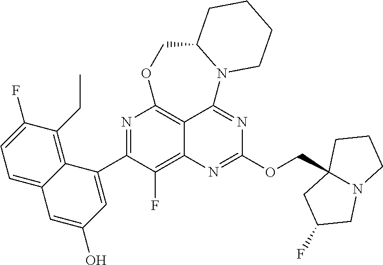 [SMILES: CCc1c(F)ccc2cc(O)cc(-c3nc4c5c(nc(OC[C@@]67CCCN6C[C@H](F)C7)nc5c3F)N3CCCC[C@H]3CO4)c12] | 5-ethyl-6-fluoro-4[(8aS)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-5-yl]naphthalen-2-ol |
| 3 | 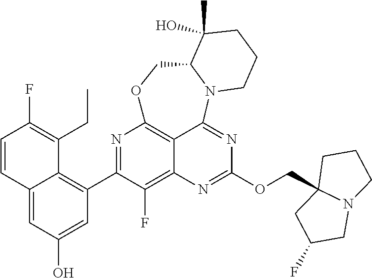 [SMILES: CCc1c(F)ccc2cc(O)cc(-c3nc4c5c(nc(OC[C@@]67CCCN6C[C@H](F)C7)nc5c3F)N3CCC[C@@](C)(O)[C@H]3CO4)c12] | (8aR,9R)-5-(8-ethyl-7-fluoro-3- hydroxynaphthalen-1-yl)-4-fluoro-2- {[(2R,7aS)-2-fluorotetrahydro-1H- pyrrolizin-7a(5H)-yl]methoxy}-9- methyl-8,8a,9,10,11,12-hexahydro-7- oxa-1,3,6,12a- tetraazabenzo[4,5]cyclohepta[1,2,3- de]naphthalen-9-ol |
| 4 | 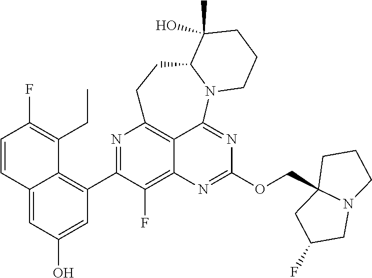 [SMILES: CCc1c(F)ccc2cc(O)cc(-c3nc4c5c(nc(OC[C@@]67CCCN6C[C@H](F)C7)nc5c3F)N3CCC[C@@](C)(O)[C@H]3CC4)c12] | (8aR,9R)-5-(8-ethyl-7-fluoro-3- hydroxynaphthalen-1-yl)-4-fluoro-2- {[(2R,7aS)-2-fluorotetrahydro-1H- pyrrolizin-7a(5H)-yl]methoxy}-9- methyl-8,8a,9,10,11,12-hexahydro- 7H-1,3,6,12a- tetraazabenzo[4,5]cyclohepta[1,2,3- de]naphthalen-9-ol |
| 5a/5b | 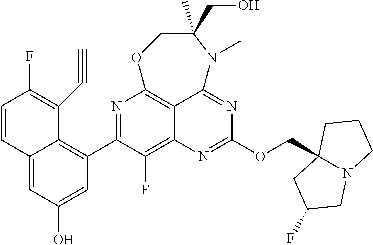 [SMILES: C#Cc1c(F)ccc2cc(O)cc(-c3nc4c5c(nc(OC[C@@]67CCCN6C[C@H](F)C7)nc5c3F)N(C)[C@](C)(CO)CO4)c12] 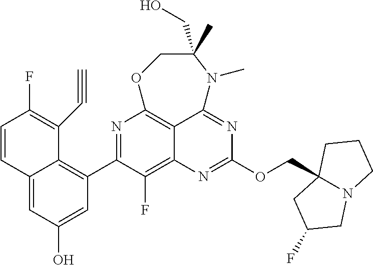 [SMILES: C#Cc1c(F)ccc2cc(O)cc(-c3nc4c5c(nc(OC[C@@]67CCCN6C[C@H](F)C7)nc5c3F)N(C)[C@@](C)(CO)CO4)c12] | 5a: 5-ethynyl-6-fluoro-4-[(9R)-4- fluoro-2-{[(2R,7aS)-2- fluorotetrahydro-1H-pyrrolizin- 7a(5H)-yl]methoxy}-9- (hydroxymethyl)-9,10-dimethyl-9,10- dihydro-8H-7-oxa-1,3,6,10- tetraazacyclohepta[1,2,3- de]naphthalen-5-yl]naphthalen-2-ol 5b: 5-ethynyl-6-fluoro-4-[(9S)-4- fluoro-2-{[(2R,7aS)-2- fluorotetrahydro-1H-pyrrolizin- 7a(5H)-yl]methoxy}-9- (hydroxymethyl)-9,10-dimethyl-9,10- dihydro-8H-7-oxa-1,3,6,10- tetraazacyclohepta[1,2,3- de]naphthalen-5-yl]naphthalen-2-ol |
| 6 | 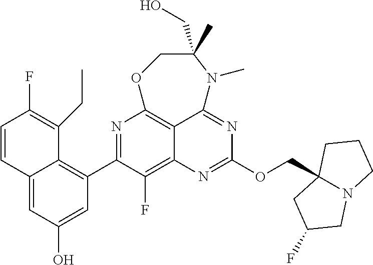 [SMILES: CCc1c(F)ccc2cc(O)cc(-c3nc4c5c(nc(OC[C@@]67CCCN6C[C@H](F)C7)nc5c3F)N(C)[C@@](C)(CO)CO4)c12] | 5-ethyl-6-fluoro-4-[(9S)-4-fluoro-2- {[(2R,7aS)-2-fluorotetrahydro-1H- pyrrolizin-7a(5H)-yl]methoxy}-9- (hydroxymethyl)-9,10-dimethyl-9,10- dihydro-8H-7-oxa-1,3,6,10- tetraazacyclohepta[1,2,3- de]naphthalen-5-yl]naphthalen-2-ol |
| 7 | 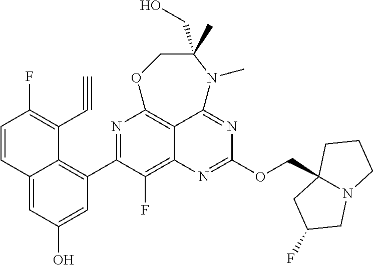 [SMILES: C#Cc1c(F)ccc2cc(O)cc(-c3nc4c5c(nc(OC[C@@]67CCCN6C[C@H](F)C7)nc5c3F)N(C)[C@@](C)(CO)CO4)c12] | 5-ethynyl-6-fluoro-4-[(9R)-4-fluoro- 2-{[(2R,7aS)-2-fluorotetrahydro-1H- pyrrolizin-7a(5H)-yl]methoxy}-9- (hydroxymethyl)-9,10-dimethyl- 7,8,9,10-tetrahydro-1,3,6,10- tetraazacyclohepta[1,2,3- de]naphthalen-5-yl]naphthalen-2-ol |
| 8 | 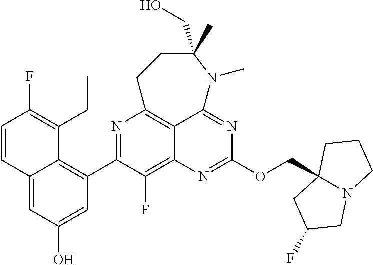 [SMILES: CCc1c(F)ccc2cc(O)cc(-c3nc4c5c(nc(OC[C@@]67CCCN6C[C@H](F)C7)nc5c3F)N(C)[C@@](C)(CO)CC4)c12] | 5-ethyl-6-fluoro-4-[(9R)-4-fluoro-2- {[(2R,7aS)-2-fluorotetrahydro-1H- pyrrolizin-7a(5H)-yl]methoxy}-9- (hydroxymethyl)-9,10-dimethyl- 7,8,9,10-tetrahydro-1,3,6,10- tetraazacyclohepta[1,2,3- de]naphthalen-5-yl]naphthalen-2-ol |
| 9 | 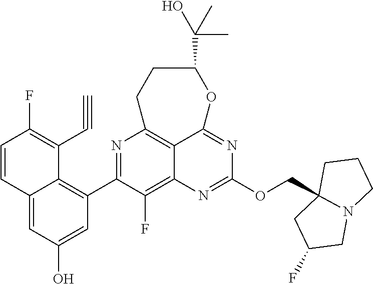 [SMILES: C#Cc1c(F)ccc2cc(O)cc(-c3nc4c5c(nc(OC[C@@]67CCCN6C[C@H](F)C7)nc5c3F)O[C@@H](C(C)(C)O)CC4)c12] | 5-ethynyl-6-fluoro-4-[(9R)-4-fluoro- 2-{[(2R,7aS)-2-fluorotetrahydro-1H- pyrrolizin-7a(5H)-yl]methoxy}-9-(2- hydroxypropan-2-yl)-8,9-dihydro- 7H-10-oxa-1,3,6- triazacyclohepta[1,2,3- de]naphthalen-5-yl]naphthalen-2-ol |
| 10 | 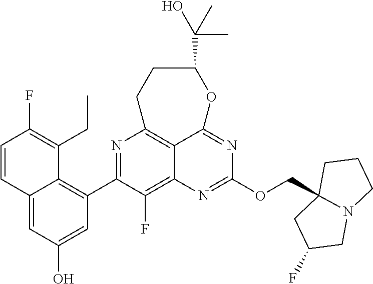 [SMILES: CCc1c(F)ccc2cc(O)cc(-c3nc4c5c(nc(OC[C@@]67CCCN6C[C@H](F)C7)nc5c3F)O[C@@H](C(C)(C)O)CC4)c12] | 5-ethyl-6-fluoro-4-[(9R)-4-fluoro-2- {[(2R,7aS)-2-fluorotetrahydro-1H- pyrrolizin-7a(5H)-yl]methoxy}-9-(2- hydroxypropan-2-yl)-8,9-dihydro- 7H-10-oxa-1,3,6- triazacyclohepta[1,2,3- de]naphthalen-5-yl]naphthalen-2-ol |
| 11 | 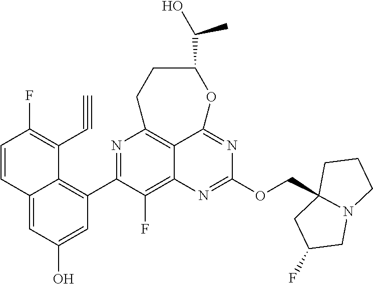 [SMILES: C#Cc1c(F)ccc2cc(O)cc(-c3nc4c5c(nc(OC[C@@]67CCCN6C[C@H](F)C7)nc5c3F)O[C@@H]([C@H](C)O)CC4)c12] | 5-ethynyl-6-fluoro-4-{(9R)-4-fluoro- 2-{[(2R,7aS)-2-fluorotetrahydro-1H- pyrrolizin-7a(5H)-yl]methoxy}-9- [(1S)-1-hydroxyethyl]-8,9-dihydro- 7H-10-oxa-1,3,6- triazacyclohepta[1,2,3- de]naphthalen-5-yl}naphthalen-2-ol |
| 12 | 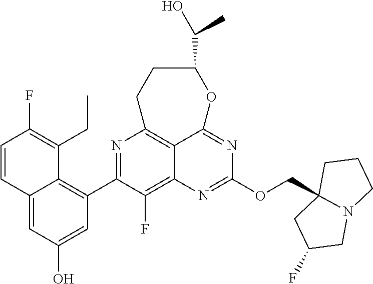 [SMILES: CCc1c(F)ccc2cc(O)cc(-c3nc4c5c(nc(OC[C@@]67CCCN6C[C@H](F)C7)nc5c3F)O[C@@H]([C@H](C)O)CC4)c12] | 5-ethyl-6-fluoro-4-{(9R)-4-fluoro-2- {[(2R,7aS)-2-fluorotetrahydro-1H- pyrrolizin-7a(5H)-yl]methoxy}-9- [(1S)-1-hydroxyethyl]-8,9-dihydro- 7H-10-oxa-1,3,6- triazacyclohepta[1,2,3- de]naphthalen-5-yl}naphthalen-2-ol |
| 13 | 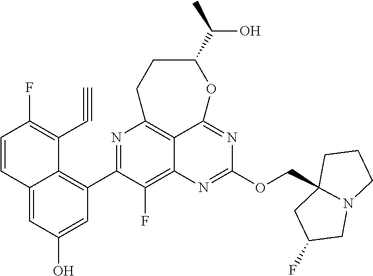 [SMILES: C#Cc1c(F)ccc2cc(O)cc(-c3nc4c5c(nc(OC[C@@]67CCCN6C[C@H](F)C7)nc5c3F)O[C@@H]([C@@H](C)O)CC4)c12] | 5-ethynyl-6-fluoro-4-{(9R)-4-fluoro- 2-{[(2R,7aS)-2-fluorotetrahydro-1H- pyrrolizin-7a(5H)-yl]methoxy}-9- [(1R)-1-hydroxyethyl]-8,9-dihydro- 7H-10-oxa-1,3,6- triazacyclohepta[1,2,3- de]naphthalen-5-yl}naphthalen-2-ol |
| 14 | 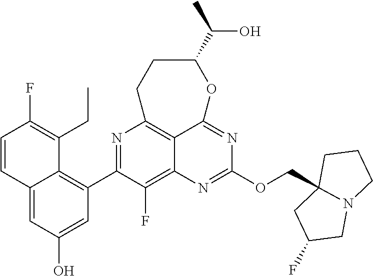 [SMILES: CCc1c(F)ccc2cc(O)cc(-c3nc4c5c(nc(OC[C@@]67CCCN6C[C@H](F)C7)nc5c3F)O[C@@H]([C@@H](C)O)CC4)c12] | 5-ethyl-6-fluoro-4-{(9R)-4-fluoro-2- {[(2R,7aS)-2-fluorotetrahydro-1H- pyrrolizin-7a(5H)-yl]methoxy}-9- [(1R)-1-hydroxyethyl]-8,9-dihydro- 7H-10-oxa-1,3,6- triazacyclohepta[1,2,3- de]naphthalen-5-yl}naphthalen-2-ol |
| 15 | 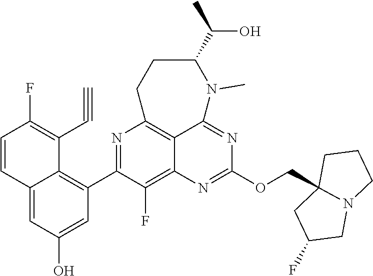 [SMILES: C#Cc1c(F)ccc2cc(O)cc(-c3nc4c5c(nc(OC[C@@]67CCCN6C[C@H](F)C7)nc5c3F)N(C)[C@@H]([C@@H](C)O)CC4)c12] | 5-ethynyl-6-fluoro-4-{(9R)-4-fluoro- 2-{[(2R,7aS)-2-fluorotetrahydro-1H- pyrrolizin-7a(5H)-yl]methoxy}-9- [(1R)-1-hydroxyethyl]-10-methyl- 7,8,9,10-tetrahydro-1,3,6,10- tetraazacyclohepta[1,2,3- de]naphthalen-5-yl}naphthalen-2-ol |
| 16 | 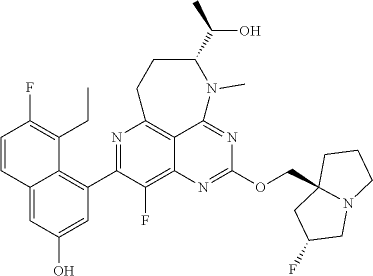 [SMILES: CCc1c(F)ccc2cc(O)cc(-c3nc4c5c(nc(OC[C@@]67CCCN6C[C@H](F)C7)nc5c3F)N(C)[C@@H]([C@@H](C)O)CC4)c12] | 5-ethyl-6-fluoro-4-{(9R)-4-fluoro-2- {[(2R,7aS)-2-fluorotetrahydro-1H- pyrrolizin-7a(5H)-yl]methoxy}-9- [(1R)-1-hydroxyethyl]-10-methyl- 7,8,9,10-tetrahydro-1,3,6,10- tetraazacyclohepta[1,2,3- de]naphthalen-5-yl}naphthalen-2-ol |
| 17 | 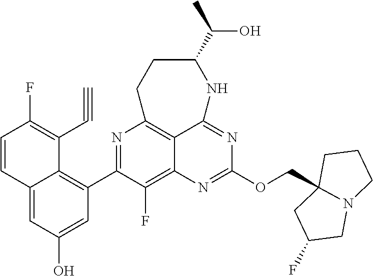 [SMILES: C#Cc1c(F)ccc2cc(O)cc(-c3nc4c5c(nc(OC[C@@]67CCCN6C[C@H](F)C7)nc5c3F)N[C@@H]([C@@H](C)O)CC4)c12] | 5-ethynyl-6-fluoro-4-{(9R)-4-fluoro- 2-{[(2R,7aS)-2-fluorotetrahydro-1H- pyrrolizin-7a(5H)-yl]methoxy}-9- [(1R)-1-hydroxyethyl]-7,8,9,10- tetrahydro-1,3,6,10- tetraazacyclohepta[1,2,3- de]naphthalen-5-yl}naphthalen-2-ol |
| 18 | 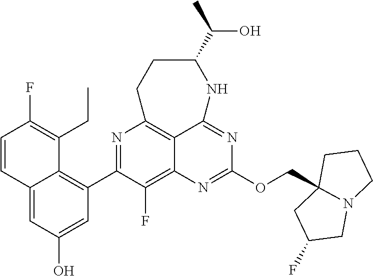 [SMILES: CCc1c(F)ccc2cc(O)cc(-c3nc4c5c(nc(OC[C@@]67CCCN6C[C@H](F)C7)nc5c3F)N[C@@H]([C@@H](C)O)CC4)c12] | 5-ethyl-6-fluoro-4-{(9R)-4-fluoro-2- {[(2R,7aS)-2-fluorotetrahydro-1H- pyrrolizin-7a(5H)-yl]methoxy}-9- [(1R)-1-hydroxyethyl]-7,8,9,10- tetrahydro-1,3,6,10- tetraazacyclohepta[1,2,3- de]naphthalen-5-yl}naphthalen-2-ol |
| 19 | 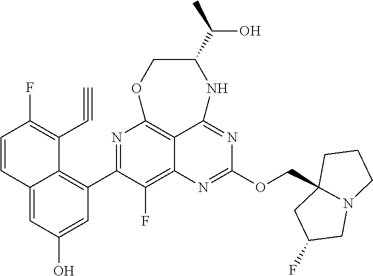 [SMILES: C#Cc1c(F)ccc2cc(O)cc(-c3nc4c5c(nc(OC[C@@]67CCCN6C[C@H](F)C7)nc5c3F)N[C@@H]([C@@H](C)O)CO4)c12] | 5-ethynyl-6-fluoro-4-{(9R)-4-fluoro- 2-{[(2R,7aS)-2-fluorotetrahydro-1H- pyrrolizin-7a(5H)-yl]methoxy}-9- [(1R)-1-hydroxyethyl]-9,10-dihydro- 8H-7-oxa-1,3,6,10- tetraazacyclohepta[1,2,3- de]naphthalen-5-yl}naphthalen-2-ol |
| 20 | 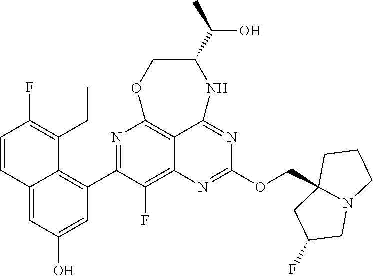 [SMILES: CCc1c(F)ccc2cc(O)cc(-c3nc4c5c(nc(OC[C@@]67CCCN6C[C@H](F)C7)nc5c3F)N[C@@H]([C@@H](C)O)CO4)c12] | 5-ethyl-6-fluoro-4-{(9R)-4-fluoro-2- {[(2R,7aS)-2-fluorotetrahydro-1H- pyrrolizin-7a(5H)-yl]methoxy}-9- [(1R)-1-hydroxyethyl]-9,10-dihydro- 8H-7-oxa-1,3,6,10- tetraazacyclohepta[1,2,3- de]naphthalen-5-yl}naphthalen-2-ol |
| 21 | 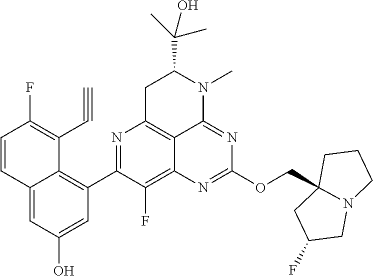 [SMILES: C#Cc1c(F)ccc2cc(O)cc(-c3nc4c5c(nc(OC[C@@]67CCCN6C[C@H](F)C7)nc5c3F)N(C)[C@@H](C(C)(C)O)C4)c12] | 5-ethynyl-6-fluoro-4-[(5R)-9-fluoro- 2-{[(2R,7aS)-2-fluorotetrahydro-1H- pyrrolizin-7a(5H)-yl]methoxy}-5-(2- hydroxypropan-2-yl)-4-methyl-5,6- dihydro-4H-pyrimido[4,5,6- de][1,6]naphthyridin-8- yl]naphthalen-2-ol |
| 22 | 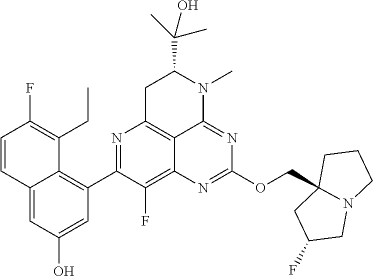 [SMILES: CCc1c(F)ccc2cc(O)cc(-c3nc4c5c(nc(OC[C@@]67CCCN6C[C@H](F)C7)nc5c3F)N(C)[C@@H](C(C)(C)O)C4)c12] | 5-ethyl-6-fluoro-4-[(5R)-9-fluoro-2- {[(2R,7aS)-2-fluorotetrahydro-1H- pyrrolizin-7a(5H)-yl]methoxy}-5-(2- hydroxypropan-2-yl)-4-methyl-5,6- dihydro-4H-pyrimido[4,5,6- de][1,6]naphthyridin-8- yl]naphthalen-2-ol |
| 23 | 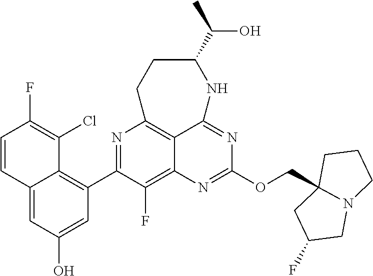 [SMILES: C[C@@H](O)[C@H]1CCc2nc(-c3cc(O)cc4ccc(F)c(Cl)c34)c(F)c3nc(OC[C@@]45CCCN4C[C@H](F)C5)nc(c23)N1] | 5-chloro-6-fluoro-4-{(9R)-4-fluoro-2- {[(2R,7aS)-2-fluorotetrahydro-1H- pyrrolizin-7a(5H)-yl]methoxy}-9- [(1R)-1-hydroxyethyl]-7,8,9,10- tetrahydro-1,3,6,10- tetraazacyclohepta[1,2,3- de]naphthalen-5-yl}naphthalen-2-ol |
| 24 | 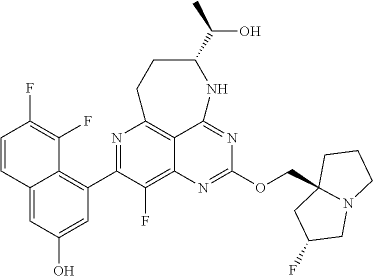 [SMILES: C[C@@H](O)[C@H]1CCc2nc(-c3cc(O)cc4ccc(F)c(F)c34)c(F)c3nc(OC[C@@]45CCCN4C[C@H](F)C5)nc(c23)N1] | 5,6-difluoro-4-{(9R)-4-fluoro-2- {[(2R,7aS)-2-fluorotetrahydro-1H- pyrrolizin-7a(5H)-yl]methoxy}-9- [(1R)-1-hydroxyethyl]-7,8,9,10- tetrahydro-1,3,6,10- tetraazacyclohepta[1,2,3- de]naphthalen-5-yl}naphthalen-2-ol |
| 25 | 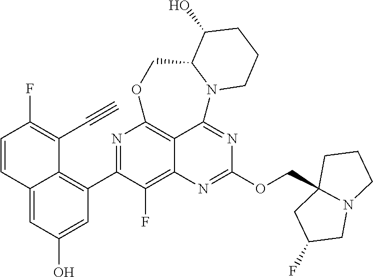 [SMILES: C#Cc1c(F)ccc2cc(O)cc(-c3nc4c5c(nc(OC[C@@]67CCCN6C[C@H](F)C7)nc5c3F)N3CCC[C@@H](O)[C@H]3CO4)c12] | (8aR,9R)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-9-ol |
| 26 | 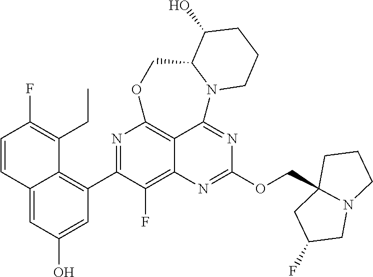 [SMILES: CCc1c(F)ccc2cc(O)cc(-c3nc4c5c(nc(OC[C@@]67CCCN6C[C@H](F)C7)nc5c3F)N3CCC[C@@H](O)[C@H]3CO4)c12] | (8aR,9R)-5-(8-ethyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-9-ol |
| 27 | 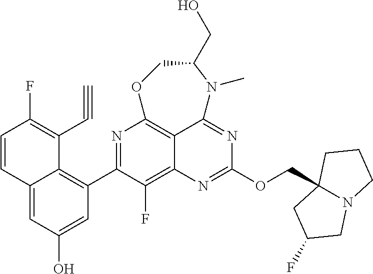 [SMILES: C#Cc1c(F)ccc2cc(O)cc(-c3nc4c5c(nc(OC[C@@]67CCCN6C[C@H](F)C7)nc5c3F)N(C)[C@@H](CO)CO4)c12] | 5-ethynyl-6-fluoro-4-[(9S)-4-fluoro- 2-{[(2R,7aS)-2-fluorotetrahydro-1H- pyrrolizin-7a(5H)-yl]methoxy}-9- (hydroxymethyl)-10-methyl-9,10- dihydro-8H-7-oxa-1,3,6,10- tetraazacyclohepta[1,2,3- de]naphthalen-5-yl]naphthalen-2-ol |
| 28 | 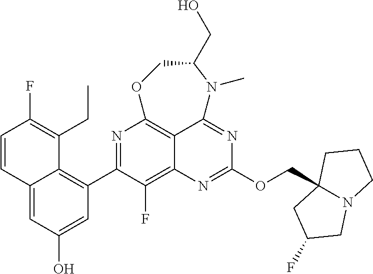 [SMILES: CCc1c(F)ccc2cc(O)cc(-c3nc4c5c(nc(OC[C@@]67CCCN6C[C@H](F)C7)nc5c3F)N(C)[C@@H](CO)CO4)c12] | 5-ethyl-6-fluoro-4-[(9S)-4-fluoro-2- {[(2R,7aS)-2-fluorotetrahydro-1H- pyrrolizin-7a(5H)-yl]methoxy}-9- (hydroxymethyl)-10-methyl-9,10- dihydro-8H-7-oxa-1,3,6,10- tetraazacyclohepta[1,2,3- de]naphthalen-5-yl]naphthalen-2-ol |
| 29 | 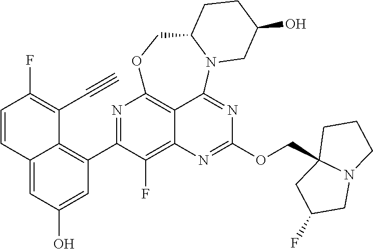 [SMILES: C#Cc1c(F)ccc2cc(O)cc(-c3nc4c5c(nc(OC[C@@]67CCCN6C[C@H](F)C7)nc5c3F)N3C[C@H](O)CC[C@H]3CO4)c12] | (8aS,11R)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-11-ol |
| 30 | 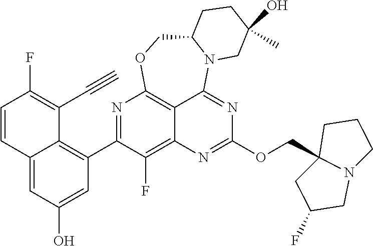 [SMILES: C#Cc1c(F)ccc2cc(O)cc(-c3nc4c5c(nc(OC[C@@]67CCCN6C[C@H](F)C7)nc5c3F)N3C[C@](C)(O)CC[C@H]3CO4)c12] | (8aS,11R)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}-11- methyl-8,8a,9,10,11,12-hexahydro-7- oxa-1,3,6,12a- tetraazabenzo[4,5]cyclohepta[1,2,3- de]naphthalen-11-ol |
| 31 | 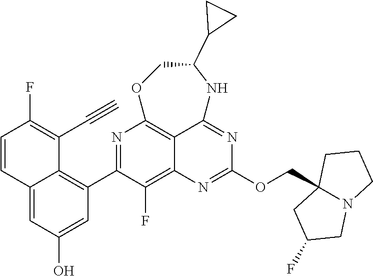 [SMILES: C#Cc1c(F)ccc2cc(O)cc(-c3nc4c5c(nc(OC[C@@]67CCCN6C[C@H](F)C7)nc5c3F)N[C@@H](C3CC3)CO4)c12] | 4-[(9S)-9-cyclopropyl-4-fluoro-2- {[(2R,7aS)-2-fluorotetrahydro-1H- pyrrolizin-7a(5H)-yl]methoxy}-9,10- dihydro-8H-7-oxa-1,3,6,10- tetraazacyclohepta[1,2,3- de]naphthalen-5-yl]-5-ethynyl-6- fluoronaphthalen-2-ol |
| 32 | 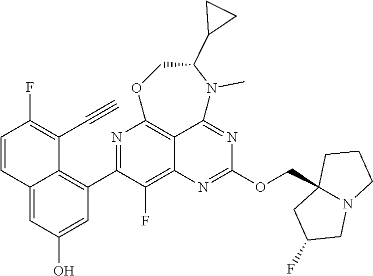 [SMILES: C#Cc1c(F)ccc2cc(O)cc(-c3nc4c5c(nc(OC[C@@]67CCCN6C[C@H](F)C7)nc5c3F)N(C)[C@@H](C3CC3)CO4)c12] | 4-[(9S)-9-cyclopropyl-4-fluoro-2- {[(2R,7aS)-2-fluorotetrahydro-1H- pyrrolizin-7a(5H)-yl]methoxy}-10- methyl-9,10-dihydro-8H-7-oxa- 1,3,6,10-tetraazacyclohepta[1,2,3- de]naphthalen-5-yl]-5-ethynyl-6- fluoronaphthalen-2-ol |
| 33 | 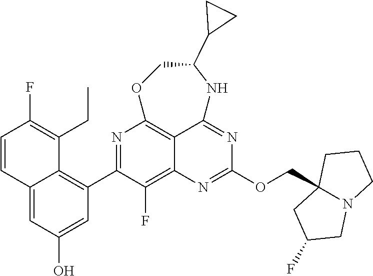 [SMILES: CCc1c(F)ccc2cc(O)cc(-c3nc4c5c(nc(OC[C@@]67CCCN6C[C@H](F)C7)nc5c3F)N[C@@H](C3CC3)CO4)c12] | 4-[(9S)-9-cyclopropyl-4-fluoro-2- {[(2R,7aS)-2-fluorotetrahydro-1H- pyrrolizin-7a(5H)-yl]methoxy}-9,10- dihydro-8H-7-oxa-1,3,6,10- tetraazacyclohepta[1,2,3- de]naphthalen-5-yl]-5-ethyl-6- fluoronaphthalen-2-ol |
| 34 | 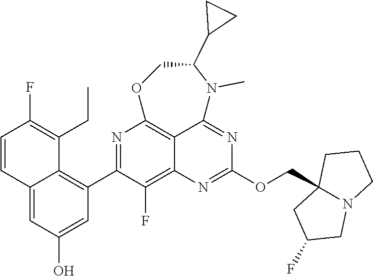 [SMILES: CCc1c(F)ccc2cc(O)cc(-c3nc4c5c(nc(OC[C@@]67CCCN6C[C@H](F)C7)nc5c3F)N(C)[C@@H](C3CC3)CO4)c12] | 4-[(9S)-9-cyclopropyl-4-fluoro-2- {[(2R,7aS)-2-fluorotetrahydro-1H- pyrrolizin-7a(5H)-yl]methoxy}-10- methyl-9,10-dihydro-8H-7-oxa- 1,3,6,10-tetraazacyclohepta[1,2,3- de]naphthalen-5-yl]-5-ethyl-6- fluoronaphthalen-2-ol |
| 35 | 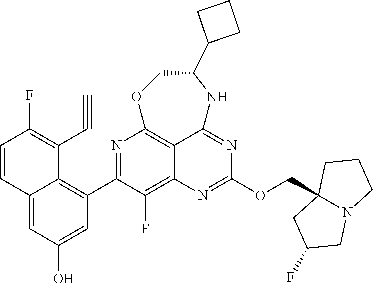 [SMILES: C#Cc1c(F)ccc2cc(O)cc(-c3nc4c5c(nc(OC[C@@]67CCCN6C[C@H](F)C7)nc5c3F)N[C@@H](C3CCC3)CO4)c12] | 4-[(9S)-9-cyclobutyl-4-fluoro-2- {[(2R,7aS)-2-fluorotetrahydro-1H- pyrrolizin-7a(5H)-yl]methoxy}-9,10- dihydro-8H-7-oxa-1,3,6,10- tetraazacyclohepta[1,2,3- de]naphthalen-5-yl]-5-ethynyl-6- fluoronaphthalen-2-ol |
| 36 | 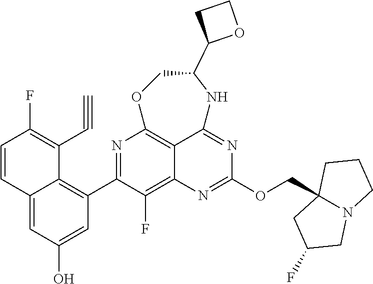 [SMILES: C#Cc1c(F)ccc2cc(O)cc(-c3nc4c5c(nc(OC[C@@]67CCCN6C[C@H](F)C7)nc5c3F)N[C@@H]([C@H]3CCO3)CO4)c12] | 5-ethynyl-6-fluoro-4-{(9R)-4-fluoro- 2-{[(2R,7aS)-2-fluorotetrahydro-1H- pyrrolizin-7a(5H)-yl]methoxy}-9- [(2R)-oxetan-2-yl]-9,10-dihydro-8H- 7-oxa-1,3,6,10- tetraazacyclohepta[1,2,3- de]naphthalen-5-yl}naphthalen-2-ol |
| 37 | 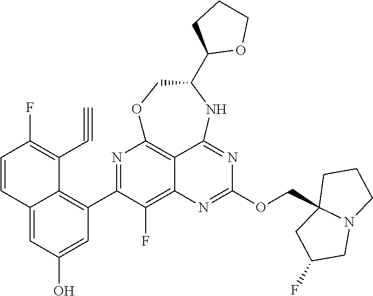 [SMILES: C#Cc1c(F)ccc2cc(O)cc(-c3nc4c5c(nc(OC[C@@]67CCCN6C[C@H](F)C7)nc5c3F)N[C@@H]([C@H]3CCCO3)CO4)c12] | 5-ethynyl-6-fluoro-4-{(9R)-4-fluoro- 2-{[(2R,7aS)-2-fluorotetrahydro-1H- pyrrolizin-7a(5H)-yl]methoxy}-9- [(2R)-oxolan-2-yl]-9,10-dihydro-8H- 7-oxa-1,3,6,10- tetraazacyclohepta[1,2,3- de]naphthalen-5-yl}naphthalen-2-ol |
| 38 | 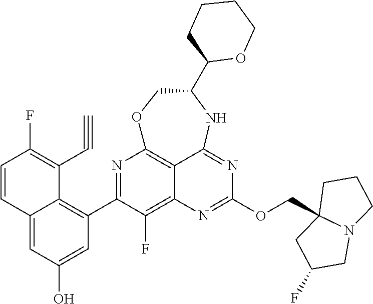 [SMILES: C#Cc1c(F)ccc2cc(O)cc(-c3nc4c5c(nc(OC[C@@]67CCCN6C[C@H](F)C7)nc5c3F)N[C@@H]([C@H]3CCCCO3)CO4)c12] | 5-ethynyl-6-fluoro-4-{(9R)-4-fluoro- 2-{[(2R,7aS)-2-fluorotetrahydro-1H- pyrrolizin-7a(5H)-yl]methoxy}-9- [(2R)-oxan-2-yl]-9,10-dihydro-8H-7- oxa-1,3,6,10- tetraazacyclohepta[1,2,3- de]naphthalen-5-yl}naphthalen-2-ol |
| 39 | 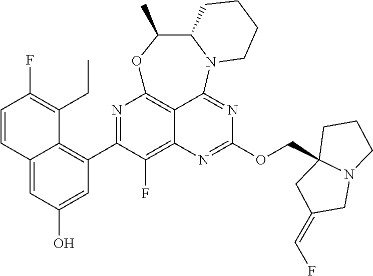 [SMILES: CCc1c(F)ccc2cc(O)cc(-c3nc4c5c(nc(OC[C@@]67CCCN6C/C(=C\F)C7)nc5c3F)N3CCCC[C@H]3[C@H](C)O4)c12] | 5-ethyl-6-fluoro-4-[(8S,8aS)-4- fluoro-2-{[(2R,7aS)-2- fluorotetrahydro-1H-pyrrolizin- 7a(5H)-yl]methoxy}-8-methyl- 8,8a,9,10,11,12-hexahydro-7-oxa- 1,3,6,12a- tetraazabenzo[4,5]cyclohepta[1,2,3- de]naphthalen-5-yl]naphthalen-2-ol |
| 40 | 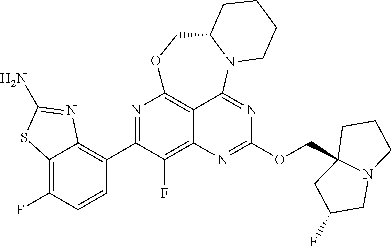 [SMILES: Nc1nc2c(-c3nc4c5c(nc(OC[C@@]67CCCN6C[C@H](F)C7)nc5c3F)N3CCCC[C@H]3CO4)ccc(F)c2s1] | 7-fluoro-4-[(8aS)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-5-yl]-1,3- benzothiazol-2-amine |
| 41 | 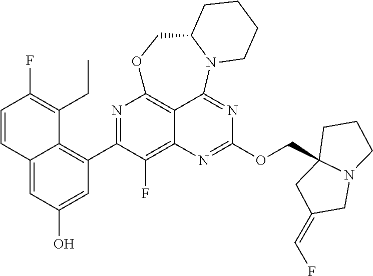 [SMILES: CCc1c(F)ccc2cc(O)cc(-c3nc4c5c(nc(OC[C@@]67CCCN6C/C(=C\F)C7)nc5c3F)N3CCCC[C@H]3CO4)c12] | 5-ethyl-6-fluoro-4-[(8aS)-4-fluoro-2- {[(2Z,7aS)-2- (fluoromethylidene)tetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-5-yl]naphthalen-2-ol |
| 42 | 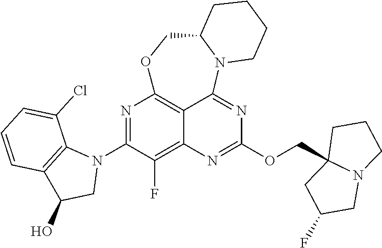 [SMILES: O[C@@H]1CN(c2nc3c4c(nc(OC[C@@]56CCCN5C[C@H](F)C6)nc4c2F)N2CCCC[C@H]2CO3)c2c(Cl)cccc21] | (3S)-7-chloro-1-[(8aS)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-5-yl]-2,3-dihydro-1H- indol-3-ol |
| 43 | 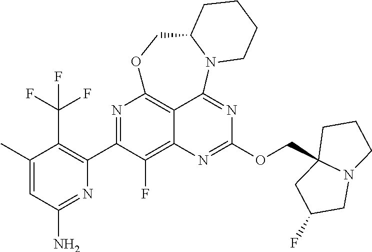 [SMILES: Cc1cc(N)nc(-c2nc3c4c(nc(OC[C@@]56CCCN5C[C@H](F)C6)nc4c2F)N2CCCC[C@H]2CO3)c1C(F)(F)F] | 6-[(8aS)-4-fluoro-2-{[(2R,7aS)-2- fluorotetrahydro-1H-pyrrolizin- 7a(5H)-yl]methoxy}-8,8a,9,10,11,12- hexahydro-7-oxa-1,3,6,12a- tetraazabenzo[4,5]cyclohepta[1,2,3- de]naphthalen-5-yl]-4-methyl-5- (trifluoromethyl)pyridin-2-amine |
| 44 | 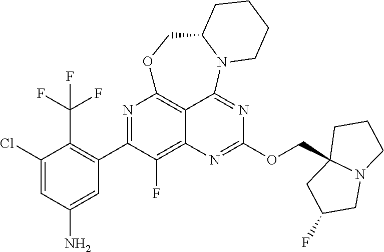 [SMILES: Nc1cc(Cl)c(C(F)(F)F)c(-c2nc3c4c(nc(OC[C@@]56CCCN5C[C@H](F)C6)nc4c2F)N2CCCC[C@H]2CO3)c1] | 3-chloro-5-[(8aS)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-5-yl]-4- (trifluoromethyl)aniline |
| 45 | 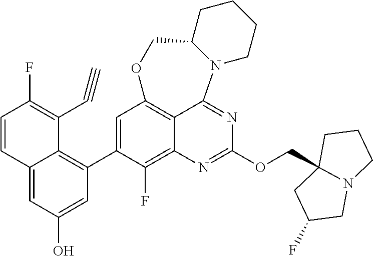 [SMILES: C#Cc1c(F)ccc2cc(O)cc(-c3cc4c5c(nc(OC[C@@]67CCCN6C[C@H](F)C7)nc5c3F)N3CCCC[C@H]3CO4)c12] | 5-ethynyl-6-fluoro-4-[(8aS)-4-fluoro- 2-{[(2R,7aS)-2-fluorotetrahydro-1H- pyrrolizin-7a(5H)-yl]methoxy}- 8,8a,9,10,11,12- hexahydropyrido[2′,1′:3,4][1,4]oxaze pino[5,6,7-de]quinazolin-5- yl]naphthalen-2-ol |
| 46 | 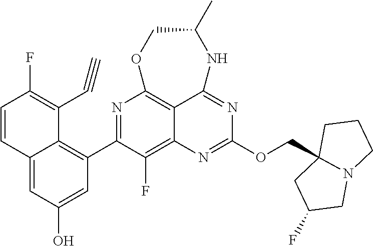 [SMILES: C#Cc1c(F)ccc2cc(O)cc(-c3nc4c5c(nc(OC[C@@]67CCCN6C[C@H](F)C7)nc5c3F)N[C@@H](C)CO4)c12] | 5-ethynyl-6-fluoro-4-[(9S)-4-fluoro- 2-{[(2R,7aS)-2-fluorotetrahydro-1H- pyrrolizin-7a(5H)-yl]methoxy}-9- methyl-9,10-dihydro-8H-7-oxa- 1,3,6,10-tetraazacyclohepta[1,2,3- de]naphthalen-5-yl]naphthalen-2-ol |
| 47 | 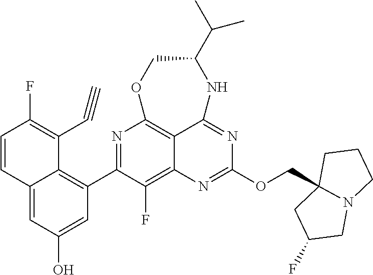 [SMILES: C#Cc1c(F)ccc2cc(O)cc(-c3nc4c5c(nc(OC[C@@]67CCCN6C[C@H](F)C7)nc5c3F)N[C@@H](C(C)C)CO4)c12] | 5-ethynyl-6-fluoro-4-[(9S)-4-fluoro- 2-{[(2R,7aS)-2-fluorotetrahydro-1H- pyrrolizin-7a(5H)-yl]methoxy}-9- (propan-2-yl)-9,10-dihydro-8H-7- oxa-1,3,6,10- tetraazacyclohepta[1,2,3- de]naphthalen-5-yl]naphthalen-2-ol |
| 48 | 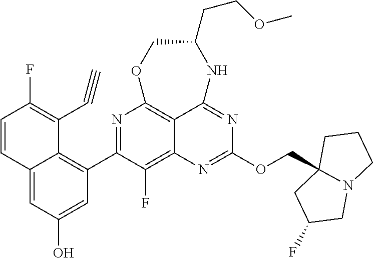 [SMILES: C#Cc1c(F)ccc2cc(O)cc(-c3nc4c5c(nc(OC[C@@]67CCCN6C[C@H](F)C7)nc5c3F)N[C@@H](CCOC)CO4)c12] | 5-ethynyl-6-fluoro-4-[(9S)-4-fluoro- 2-{[(2R,7aS)-2-fluorotetrahydro-1H- pyrrolizin-7a(5H)-yl]methoxy}-9-(2- methoxyethyl)-9,10-dihydro-8H-7- oxa-1,3,6,10- tetraazacyclohepta[1,2,3- de]naphthalen-5-yl]naphthalen-2-ol |
| 49 | 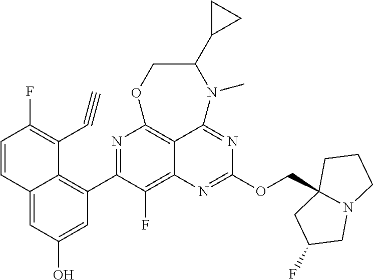 [SMILES: C#Cc1c(F)ccc2cc(O)cc(-c3nc4c5c(nc(OC[C@@]67CCCN6C[C@H](F)C7)nc5c3F)N(C)C(C3CC3)CO4)c12] | 4-(9-cyclopropyl-4-fluoro-2- (((2R,7aS)-2-fluorotetrahydro-1H- pyrrolizin-7a(5H)-yl)methoxy)-10- methyl-9,10-dihydro-8H-7-oxa- 1,3,6,10- tetraazacyclohepta[de]naphthalen-5- yl)-5-ethynyl-6-fluoronaphthalen-2- ol |
| 50 | 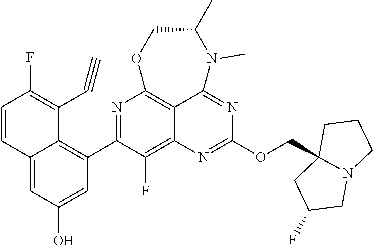 [SMILES: C#Cc1c(F)ccc2cc(O)cc(-c3nc4c5c(nc(OC[C@@]67CCCN6C[C@H](F)C7)nc5c3F)N(C)[C@@H](C)CO4)c12] | 5-ethynyl-6-fluoro-4-[(9S)-4-fluoro- 2-{[(2R,7aS)-2-fluorotetrahydro-1H- pyrrolizin-7a(5H)-yl]methoxy}-9,10- dimethyl-9,10-dihydro-8H-7-oxa- 1,3,6,10-tetraazacyclohepta[1,2,3- de]naphthalen-5-yl]naphthalen-2-ol |
| 51 | 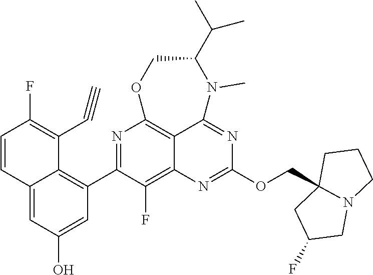 [SMILES: C#Cc1c(F)ccc2cc(O)cc(-c3nc4c5c(nc(OC[C@@]67CCCN6C[C@H](F)C7)nc5c3F)N(C)[C@@H](C(C)C)CO4)c12] | 5-ethynyl-6-fluoro-4-[(9S)-4-fluoro- 2-{[(2R,7aS)-2-fluorotetrahydro-1H- pyrrolizin-7a(5H)-yl]methoxy}-10- methyl-9-(propan-2-yl)-9,10-dihydro- 8H-7-oxa-1,3,6,10- tetraazacyclohepta[1,2,3- de]naphthalen-5-yl]naphthalen-2-ol |
| 52 | 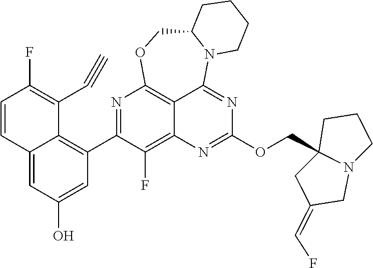 [SMILES: C#Cc1c(F)ccc2cc(O)cc(-c3nc4c5c(nc(OC[C@@]67CCCN6C/C(=C\F)C7)nc5c3F)N3CCCC[C@H]3CO4)c12] | 5-ethynyl-6-fluoro-4-[(8aS)-4-fluoro- 2-{[(2Z,7aS)-2- (fluoromethylidene)tetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-5-yl]naphthalen-2-ol |
| 53 | 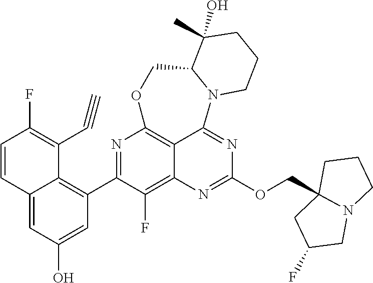 [SMILES: C#Cc1c(F)ccc2cc(O)cc(-c3nc4c5c(nc(OC[C@@]67CCCN6C[C@H](F)C7)nc5c3F)N3CCC[C@@](C)(O)[C@H]3CO4)c12] | (8aR,9R)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}-9- methyl-8,8a,9,10,11,12-hexahydro-7- oxa-1,3,6,12a- tetraazabenzo[4,5]cyclohepta[1,2,3- de]naphthalen-9-ol |
| 54 | 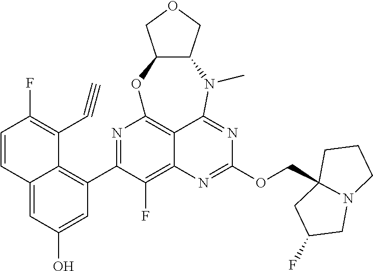 [SMILES: C#Cc1c(F)ccc2cc(O)cc(-c3nc4c5c(nc(OC[C@@]67CCCN6C[C@H](F)C7)nc5c3F)N(C)[C@H]3COC[C@@H]3O4)c12] | 5-ethynyl-6-fluoro-4-[(7aR,10aS)-4- fluoro-2-{[(2R,7aS)-2- fluorotetrahydro-1H-pyrrolizin- 7a(5H)-yl]methoxy}-11-methyl- 7a,8,10a,11-tetrahydro-10H-7,9- dioxa-1,3,6,11-tetraazanaphtho[1,8- fg]azulen-5-yl]naphthalen-2-ol |
| 55 | 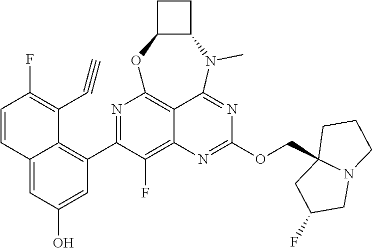 [SMILES: C#Cc1c(F)ccc2cc(O)cc(-c3nc4c5c(nc(OC[C@@]67CCCN6C[C@H](F)C7)nc5c3F)N(C)[C@H]3CC[C@@H]3O4)c12] | 5-ethynyl-6-fluoro-4-[(7aS,9aS)-4- fluoro-2-{[(2R,7aS)-2- fluorotetrahydro-1H-pyrrolizin- 7a(5H)-yl]methoxy}-10-methyl- 8,9,9a,10-tetrahydro-7aH-7-oxa- 1,3,6,10- tetraazacyclobuta[5,6]cyclohepta[1,2, 3-de]naphthalen-5-yl]naphthalen-2-ol |
| 56 | 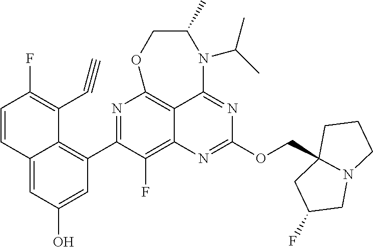 [SMILES: C#Cc1c(F)ccc2cc(O)cc(-c3nc4c5c(nc(OC[C@@]67CCCN6C[C@H](F)C7)nc5c3F)N(C(C)C)[C@@H](C)CO4)c12] | 5-ethynyl-6-fluoro-4-[(9S)-4-fluoro- 2-{[(2R,7aS)-2-fluorotetrahydro-1H- pyrrolizin-7a(5H)-yl]methoxy}-9- methyl-10-(propan-2-yl)-9,10- dihydro-8H-7-oxa-1,3,6,10- tetraazacyclohepta[1,2,3- de]naphthalen-5-yl]naphthalen-2-ol |
| 57 | 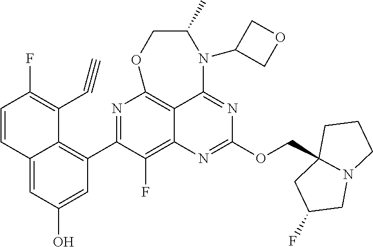 [SMILES: C#Cc1c(F)ccc2cc(O)cc(-c3nc4c5c(nc(OC[C@@]67CCCN6C[C@H](F)C7)nc5c3F)N(C3COC3)[C@@H](C)CO4)c12] | 5-ethynyl-6-fluoro-4-[(9S)-4-fluoro- 2-{[(2R,7aS)-2-fluorotetrahydro-1H- pyrrolizin-7a(5H)-yl]methoxy}-9- methyl-10-(oxetan-3-yl)-9,10- dihydro-8H-7-oxa-1,3,6,10- tetraazacyclohepta[1,2,3- de]naphthalen-5-yl]naphthalen-2-ol |
| 58 | 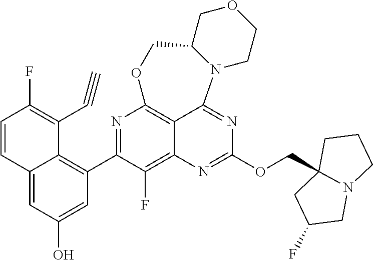 [SMILES: C#Cc1c(F)ccc2cc(O)cc(-c3nc4c5c(nc(OC[C@@]67CCCN6C[C@H](F)C7)nc5c3F)N3CCOC[C@H]3CO4)c12] | 5-ethynyl-6-fluoro-4-[(8aS)-4-fluoro- 2-{[(2R,7aS)-2-fluorotetrahydro-1H- pyrrolizin-7a(5H)-yl]methoxy}- 8a,9,11,12-tetrahydro-8H-7,10-dioxa- 1,3,6,12a- tetraazabenzo[4,5]cyclohepta[1,2,3- de]naphthalen-5-yl]naphthalen-2-ol |
| 59 | 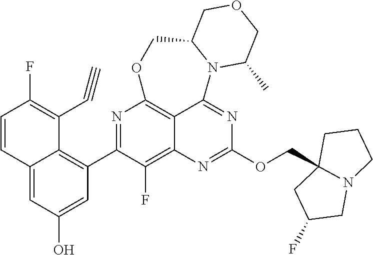 [SMILES: C#Cc1c(F)ccc2cc(O)cc(-c3nc4c5c(nc(OC[C@@]67CCCN6C[C@H](F)C7)nc5c3F)N3[C@@H](COC[C@@H]3C)CO4)c12] | 5-ethynyl-6-fluoro-4-[(8aS,12S)-4- fluoro-2-{[(2R,7aS)-2- fluorotetrahydro-1H-pyrrolizin- 7a(5H)-yl]methoxy}-12-methyl- 8a,9,11,12-tetrahydro-8H-7,10-dioxa- 1,3,6,12a- tetraazabenzo[4,5]cyclohepta[1,2,3- de]naphthalen-5-yl]naphthalen-2-ol |
| 60 | 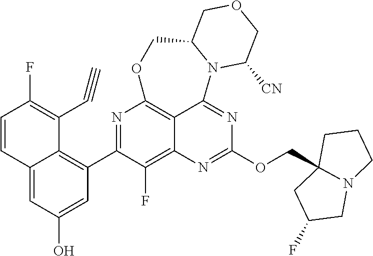 [SMILES: C#Cc1c(F)ccc2cc(O)cc(-c3nc4c5c(nc(OC[C@@]67CCCN6C[C@H](F)C7)nc5c3F)N3[C@@H](COC[C@@H]3C#N)CO4)c12] | (8aS,12S)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8a,9,11,12-tetrahydro-8H-7,10-dioxa- 1,3,6,12a- tetraazabenzo[4,5]cyclohepta[1,2,3- de]naphthalene-12-carbonitrile |
| 61 | 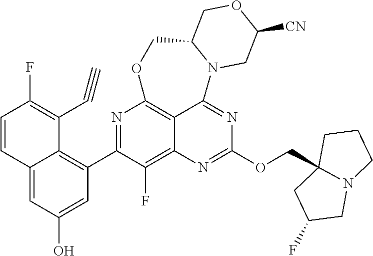 [SMILES: C#Cc1c(F)ccc2cc(O)cc(-c3nc4c5c(nc(OC[C@@]67CCCN6C[C@H](F)C7)nc5c3F)N3C[C@H](C#N)OC[C@H]3CO4)c12] | (8aS,11R)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8a,9,11,12-tetrahydro-8H-7,10-dioxa- 1,3,6,12a- tetraazabenzo[4,5]cyclohepta[1,2,3- de]naphthalene-11-carbonitrile |
| 62 | 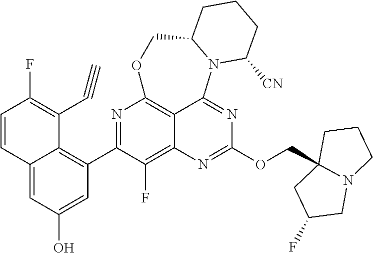 [SMILES: C#Cc1c(F)ccc2cc(O)cc(-c3nc4c5c(nc(OC[C@@]67CCCN6C[C@H](F)C7)nc5c3F)N3[C@@H](CCC[C@@H]3C#N)CO4)c12] | (8aS,12R)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalene-12-carbonitrile |
| 63 | 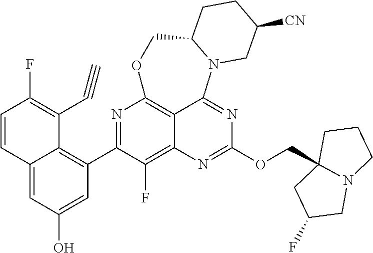 [SMILES: C#Cc1c(F)ccc2cc(O)cc(-c3nc4c5c(nc(OC[C@@]67CCCN6C[C@H](F)C7)nc5c3F)N3C[C@H](C#N)CC[C@H]3CO4)c12] | (8aS,11R)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalene-11-carbonitrile |
| 64 | 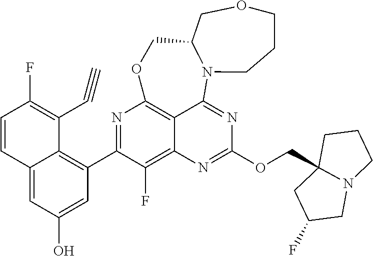 [SMILES: C#Cc1c(F)ccc2cc(O)cc(-c3nc4c5c(nc(OC[C@@]67CCCN6C[C@H](F)C7)nc5c3F)N3CCCOC[C@H]3CO4)c12] | 5-ethynyl-6-fluoro-4-((S)-1-fluoro- 12-(((2R,7aS)-2-fluorotetrahydro-1H- pyrrolizin-7a(5H)-yl)methoxy)- 5a,6,9,10-tetrahydro-5H,8H-4,7- dioxa-3,10a,11,13- tetraazanaphtho[1,8-ab]heptalen-2- yl)naphthalen-2-ol |
| 65 | 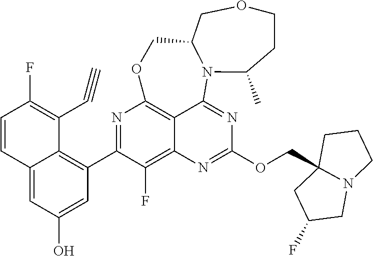 [SMILES: C#Cc1c(F)ccc2cc(O)cc(-c3nc4c5c(nc(OC[C@@]67CCCN6C[C@H](F)C7)nc5c3F)N3[C@@H](COCC[C@@H]3C)CO4)c12] | 5-ethynyl-6-fluoro-4-((5aS,10S)-1- fluoro-12-(((2R,7aS)-2- fluorotetrahydro-1H-pyrrolizin- 7a(5H)-yl)methoxy)-10-methyl- 5a,6,9,10-tetrahydro-5H,8H-4,7- dioxa-3,10a,11,13- tetraazanaphtho[1,8-ab]heptalen-2- yl)naphthalen-2-ol |
| 66 | 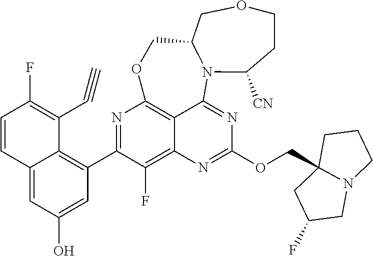 [SMILES: C#Cc1c(F)ccc2cc(O)cc(-c3nc4c5c(nc(OC[C@@]67CCCN6C[C@H](F)C7)nc5c3F)N3[C@@H](COCC[C@@H]3C#N)CO4)c12] | (8aS,13R)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8a,9,12,13-tetrahydro-8H,11H-7,10- dioxa-1,3,6,13a-tetraazanaphtho[1,8- ab]heptalene-13-carbonitrile |
| 67 | 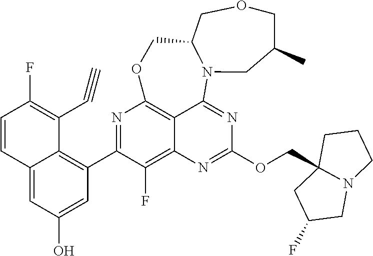 [SMILES: C#Cc1c(F)ccc2cc(O)cc(-c3nc4c5c(nc(OC[C@@]67CCCN6C[C@H](F)C7)nc5c3F)N3C[C@H](C)COC[C@H]3CO4)c12] | 5-ethynyl-6-fluoro-4-((5aS,9S)-1- fluoro-12-(((2R,7aS)-2- fluorotetrahydro-1H-pyrrolizin- 7a(5H)-yl)methoxy)-9-methyl- 5a,6,9,10-tetrahydro-5H,8H-4,7- dioxa-3,10a,11,13- tetraazanaphtho[1,8-ab]heptalen-2- yl)naphthalen-2-ol |
| 68 | 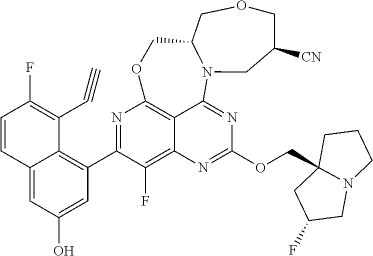 [SMILES: C#Cc1c(F)ccc2cc(O)cc(-c3nc4c5c(nc(OC[C@@]67CCCN6C[C@H](F)C7)nc5c3F)N3C[C@H](C#N)COC[C@H]3CO4)c12] | (8aS,12R)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8a,9,12,13-tetrahydro-8H,11H-7,10- dioxa-1,3,6,13a-tetraazanaphtho[1,8- ab]heptalene-12-carbonitrile |
| 69 | 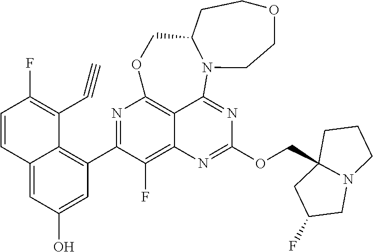 [SMILES: C#Cc1c(F)ccc2cc(O)cc(-c3nc4c5c(nc(OC[C@@]67CCCN6C[C@H](F)C7)nc5c3F)N3CCOCC[C@H]3CO4)c12] | 5-ethynyl-6-fluoro-4-((S)-1-fluoro- 12-(((2R,7aS)-2-fluorotetrahydro-1H- pyrrolizin-7a(5H)-yl)methoxy)- 5,5a,6,7,9,10-hexahydro-4,8-dioxa- 3,10a,11,13-tetraazanaphtho[1,8- ab]heptalen-2-yl)naphthalen-2-ol |
| 70 | 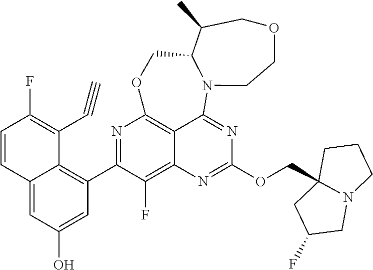 [SMILES: C#Cc1c(F)ccc2cc(O)cc(-c3nc4c5c(nc(OC[C@@]67CCCN6C[C@H](F)C7)nc5c3F)N3CCOC[C@H](C)[C@H]3CO4)c12] | 5-ethynyl-6-fluoro-4-((5aS,6R)-1- fluoro-12-(((2R,7aS)-2- fluorotetrahydro-1H-pyrrolizin- 7a(5H)-yl)methoxy)-6-methyl- 5,5a,6,7,9,10-hexahydro-4,8-dioxa- 3,10a,11,13-tetraazanaphtho[1,8- ab]heptalen-2-yl)naphthalen-2-ol |
| 71 | 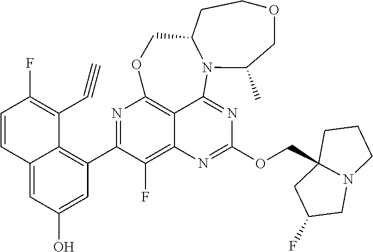 [SMILES: C#Cc1c(F)ccc2cc(O)cc(-c3nc4c5c(nc(OC[C@@]67CCCN6C[C@H](F)C7)nc5c3F)N3[C@@H](CCOC[C@@H]3C)CO4)c12] | 5-ethynyl-6-fluoro-4-((5aS,10S)-1- fluoro-12-(((2R,7aS)-2- fluorotetrahydro-1H-pyrrolizin- 7a(5H)-yl)methoxy)-10-methyl- 5,5a,6,7,9,10-hexahydro-4,8-dioxa- 3,10a,11,13-tetraazanaphtho[1,8- ab]heptalen-2-yl)naphthalen-2-ol |
| 72 | 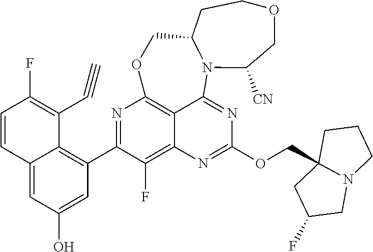 [SMILES: C#Cc1c(F)ccc2cc(O)cc(-c3nc4c5c(nc(OC[C@@]67CCCN6C[C@H](F)C7)nc5c3F)N3[C@@H](CCOC[C@@H]3C#N)CO4)c12] | (8aS,13S)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,12,13-hexahydro-7,11- dioxa-1,3,6,13a-tetraazanaphtho[1,8- ab]heptalene-13-carbonitrile |
| 73 | 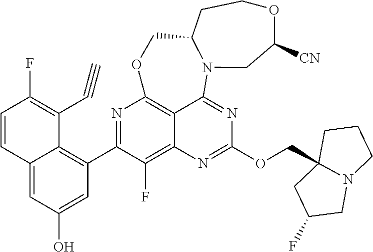 [SMILES: C#Cc1c(F)ccc2cc(O)cc(-c3nc4c5c(nc(OC[C@@]67CCCN6C[C@H](F)C7)nc5c3F)N3C[C@H](C#N)OCC[C@H]3CO4)c12] | (8aS,12R)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,12,13-hexahydro-7,11- dioxa-1,3,6,13a-tetraazanaphtho[1,8- ab]heptalene-12-carbonitrile |
| 74 | 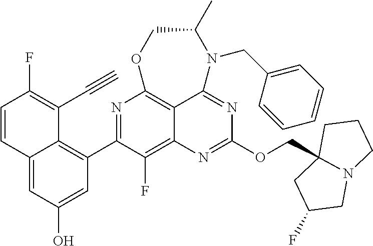 [SMILES: C#Cc1c(F)ccc2cc(O)cc(-c3nc4c5c(nc(OC[C@@]67CCCN6C[C@H](F)C7)nc5c3F)N(Cc3ccccc3)[C@@H](C)CO4)c12] | 4-[(9S)-10-benzyl-4-fluoro-2- {[(2R,7aS)-2-fluorotetrahydro-1H- pyrrolizin-7a(5H)-yl]methoxy}-9- methyl-9,10-dihydro-8H-7-oxa- 1,3,6,10-tetraazacyclohepta[1,2,3- de]naphthalen-5-yl]-5-ethynyl-6- fluoronaphthalen-2-ol |
| 75 | 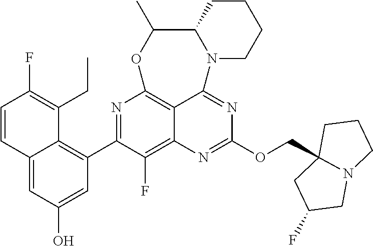 [SMILES: CCc1c(F)ccc2cc(O)cc(-c3nc4c5c(nc(OC[C@@]67CCCN6C[C@H](F)C7)nc5c3F)N3CCCC[C@H]3C(C)O4)c12] | 5-ethyl-6-fluoro-4-((5aS)-1-fluoro- 11-(((2R,7aS)-2-fluorotetrahydro-1H- pyrrolizin-7a(5H)-yl)methoxy)-5- methyl-5,5a,6,7,8,9-hexahydro-4- oxa-3,9a, 10,12- tetraazabenzo[4,5]cyclohepta[1,2,3- de]naphthalen-2-yl)naphthalen-2-ol |
| 76 | 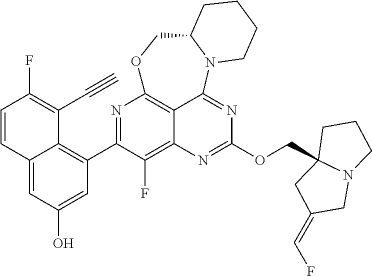 [SMILES: C#Cc1c(F)ccc2cc(O)cc(-c3nc4c5c(nc(OC[C@@]67CCCN6C/C(=C\F)C7)nc5c3F)N3CCCC[C@H]3CO4)c12] | 5-ethynyl-6-fluoro-4-[(8aS)-4-fluoro- 2-{[(2Z,7aS)-2- (fluoromethylidene)tetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-5-yl]naphthalen-2-ol |
| 77 | 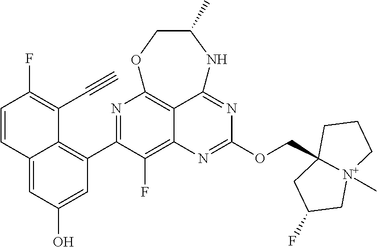 [SMILES: C#Cc1c(F)ccc2cc(O)cc(-c3nc4c5c(nc(OC[C@@]67CCC[N+]6(C)C[C@H](F)C7)nc5c3F)N[C@@H](C)CO4)c12] | (2R,7aS)-7a-({[(9S)-5-(8-ethynyl-7- fluoro-3-hydroxynaphthalen-1-yl)-4- fluoro-9-methyl-9,10-dihydro-8H-7- oxa-1,3,6,10- tetraazacyclohepta[1,2,3- de]naphthalen-2-yl]oxy}methyl)-2- fluoro-4-methylhexahydro-1H- pyrrolizin-4-ium |
| 78 | 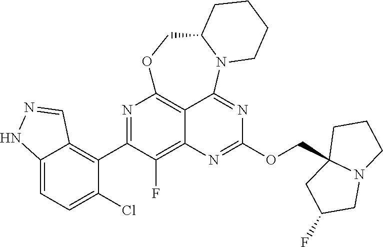 [SMILES: Fc1c(-c2c(Cl)ccc3[nH]ncc23)nc2c3c(nc(OC[C@@]45CCCN4C[C@H](F)C5)nc13)N1CCCC[C@H]1CO2] | (8aS)-5-(5-chloro-1H-indazol-4-yl)- 4-fluoro-2-{[(2R,7aS)-2- fluorotetrahydro-1H-pyrrolizin- 7a(5H)-yl]methoxy}-8,8a,9,10,11,12- hexahydro-7-oxa-1,3,6,12a- tetraazabenzo[4,5]cyclohepta[1,2,3- de]naphthalene |
| 79 | 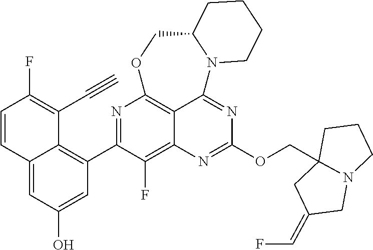 [SMILES: C#Cc1c(F)ccc2cc(O)cc(-c3nc4c5c(nc(OCC67CCCN6C/C(=C/F)C7)nc5c3F)N3CCCC[C@H]3CO4)c12] | 5-ethynyl-6-fluoro-4-[(8aS)-4-fluoro- 2-{[(2E)-2- (fluoromethylidene)tetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-5-yl]naphthalen-2-ol |
| 80 | 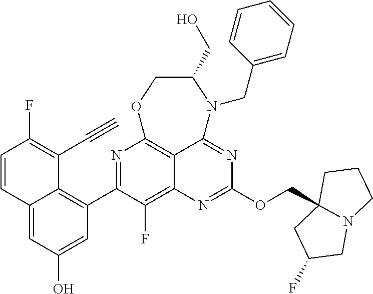 [SMILES: C#Cc1c(F)ccc2cc(O)cc(-c3nc4c5c(nc(OC[C@@]67CCCN6C[C@H](F)C7)nc5c3F)N(Cc3ccccc3)[C@@H](CO)CO4)c12] | 4-[(9S)-10-benzyl-4-fluoro-2- {[(2R,7aS)-2-fluorotetrahydro-1H- pyrrolizin-7a(5H)-yl]methoxy}-9- (hydroxymethyl)-9,10-dihydro-8H-7- oxa-1,3,6,10- tetraazacyclohepta[1,2,3- de]naphthalen-5-yl]-5-ethynyl-6- fluoronaphthalen-2-ol |
| 81 | 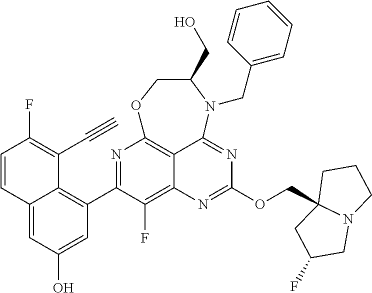 [SMILES: C#Cc1c(F)ccc2cc(O)cc(-c3nc4c5c(nc(OC[C@@]67CCCN6C[C@H](F)C7)nc5c3F)N(Cc3ccccc3)[C@H](CO)CO4)c12] | 4-[(9R)-10-benzyl-4-fluoro-2- {[(2R,7aS)-2-fluorotetrahydro-1H- pyrrolizin-7a(5H)-yl]methoxy}-9- (hydroxymethyl)-9,10-dihydro-8H-7- oxa-1,3,6,10- tetraazacyclohepta[1,2,3- de]naphthalen-5-yl]-5-ethynyl-6- fluoronaphthalen-2-ol |
| 82 | 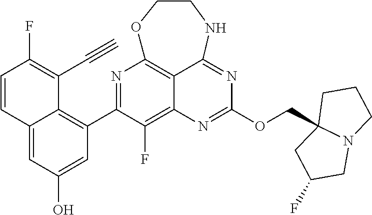 [SMILES: C#Cc1c(F)ccc2cc(O)cc(-c3nc4c5c(nc(OC[C@@]67CCCN6C[C@H](F)C7)nc5c3F)NCCO4)c12] | 5-ethynyl-6-fluoro-4-(4-fluoro-2- {[(2R,7aS)-2-fluorotetrahydro-1H- pyrrolizin-7a(5H)-yl]methoxy}-9,10- dihydro-8H-7-oxa-1,3,6,10- tetraazacyclohepta[1,2,3- de]naphthalen-5-yl)naphthalen-2-ol |
| 83 | 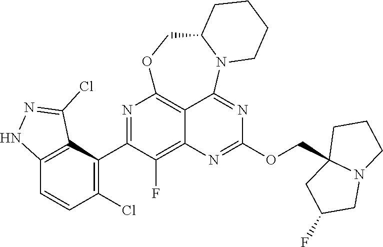 | (5P,8aS)-5-(3,5-dichloro-1H-indazol- 4-yl)-4-fluoro-2-{[(2R,7aS)-2- fluorotetrahydro-1H-pyrrolizin- 7a(5H)-yl]methoxy}-8,8a,9,10,11,12- hexahydro-7-oxa-1,3,6,12a- tetraazabenzo\|4,5\|cyclohepta\|1,2,3- de]naphthalene |
| 84 | 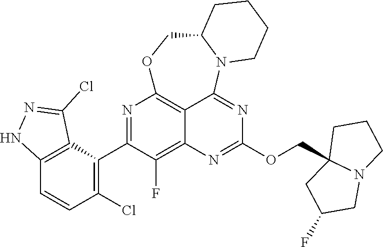 | (5M,8aS)-5-(3,5-dichloro-1H- indazol-4-yl)-4-fluoro-2-{[(2R,7aS)- 2-fluorotetrahydro-1H-pyrrolizin- 7a(5H)-yl]methoxy}-8,8a,9,10,11,12- hexahydro-7-oxa-1,3,6,12a- tetraazabenzo[4,5]cyclohepta[1,2,3- de]naphthalene |
| 85 | 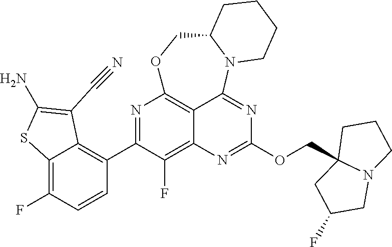 [SMILES: N#Cc1c(N)sc2c(F)ccc(-c3nc4c5c(nc(OC[C@@]67CCCN6C[C@H](F)C7)nc5c3F)N3CCCC[C@H]3CO4)c12] | 2-amino-7-fluoro-4-[(8aS)-4-fluoro- 2-{[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-5-yl]-1- benzothiophene-3-carbonitrile |
| 86 | 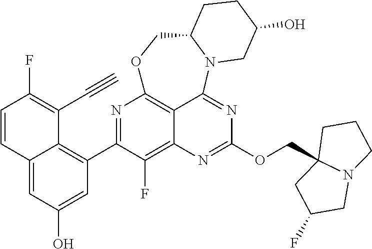 [SMILES: C#Cc1c(F)ccc2cc(O)cc(-c3nc4c5c(nc(OC[C@@]67CCCN6C[C@H](F)C7)nc5c3F)N3C[C@@H](O)CC[C@H]3CO4)c12] | (8aS,11S)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-11-7ol |
| 87 | 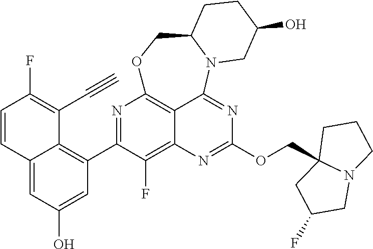 [SMILES: C#Cc1c(F)ccc2cc(O)cc(-c3nc4c5c(nc(OC[C@@]67CCCN6C[C@H](F)C7)nc5c3F)N3C[C@H](O)CC[C@@H]3CO4)c12] | (8aR,11R)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-11-ol |
| 88 | 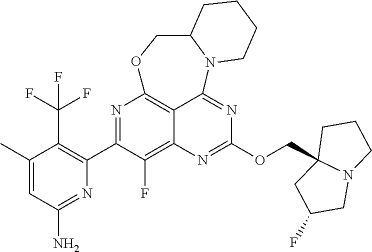 [SMILES: Cc1cc(N)nc(-c2nc3c4c(nc(OC[C@@]56CCCN5C[C@H](F)C6)nc4c2F)N2CCCCC2CO3)c1C(F)(F)F] | 6-(1-fluoro-11-(((2R,7aS)-2- fluorotetrahydro-1H-pyrrolizin- 7a(5H)-yl)methoxy)-5,5a,6,7,8,9- hexahydro-4-oxa-3,9a,10,12- tetraazabenzo[4,5]cyclohepta[1,2,3- de]naphthalen-2-yl)-4-methyl-5- (trifluoromethyl)pyridin-2-amine |
| 89 | 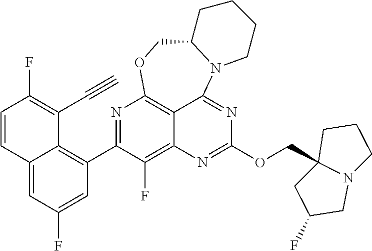 [SMILES: C#Cc1c(F)ccc2cc(F)cc(-c3nc4c5c(nc(OC[C@@]67CCCN6C[C@H](F)C7)nc5c3F)N3CCCC[C@H]3CO4)c12] | (8aS)-5-(8-ethynyl-3,7- difluoronaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalene |
| 90 | 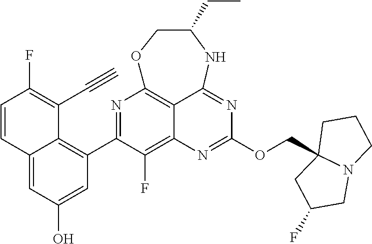 [SMILES: C#Cc1c(F)ccc2cc(O)cc(-c3nc4c5c(nc(OC[C@@]67CCCN6C[C@H](F)C7)nc5c3F)N[C@@H](CC)CO4)c12] | 4-[(9S)-9-ethyl-4-fluoro-2- {[(2R,7aS)-2-fluorotetrahydro-1H- pyrrolizin-7a(5H)-yl]methoxy}-9,10- dihydro-8H-7-oxa-1,3,6,10- tetraazacyclohepta[1,2,3- de]naphthalen-5-yl]-5-ethynyl-6- fluoronaphthalen-2-ol |
| 91 | 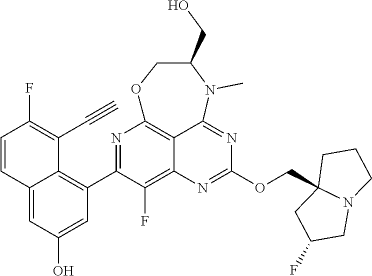 [SMILES: C#Cc1c(F)ccc2cc(O)cc(-c3nc4c5c(nc(OC[C@@]67CCCN6C[C@H](F)C7)nc5c3F)N(C)[C@H](CO)CO4)c12] | 5-ethynyl-6-fluoro-4-[(9R)-4-fluoro- 2-{[(2R,7aS)-2-fluorotetrahydro-1H- pyrrolizin-7a(5H)-yl]methoxy}-9- (hydroxymethyl)-10-methyl-9,10- dihydro-8H-7-oxa-1,3,6,10- tetraazacyclohepta[1,2,3- de]naphthalen-5-yl]naphthalen-2-ol |
| 92 | 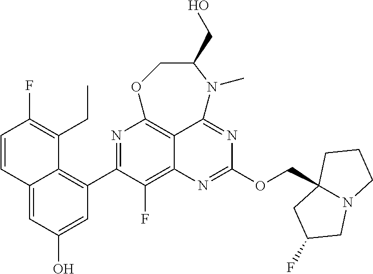 [SMILES: CCc1c(F)ccc2cc(O)cc(-c3nc4c5c(nc(OC[C@@]67CCCN6C[C@H](F)C7)nc5c3F)N(C)[C@H](CO)CO4)c12] | 5-ethyl-6-fluoro-4-[(9R)-4-fluoro-2- {[(2R,7aS)-2-fluorotetrahydro-1H- pyrrolizin-7a(5H)-yl]methoxy}-9- (hydroxymethyl)-10-methyl-9,10- dihydro-8H-7-oxa-1,3,6,10- tetraazacyclohepta[1,2,3- de]naphthalen-5-yl]naphthalen-2-ol |
| 93 | 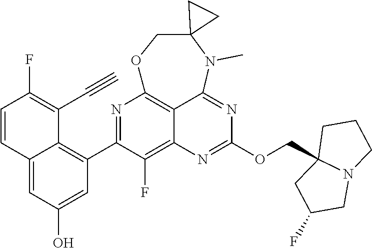 [SMILES: C#Cc1c(F)ccc2cc(O)cc(-c3nc4c5c(nc(OC[C@@]67CCCN6C[C@H](F)C7)nc5c3F)N(C)C3(CC3)CO4)c12] | 5-ethynyl-6-fluoro-4-(4′-fluoro-2′- {[(2R,7aS)-2-fluorotetrahydro-1H- pyrrolizin-7a(5H)-yl\|methoxy}-10′- methyl-8′H,10′H- spiro[cyclopropane-1,9′- [7]oxa[1,3,6,10]tetraazacyclohepta[1, 2,3-de]naphthalen]-5′-yl)naphthalen- 2-ol |
| 94 | 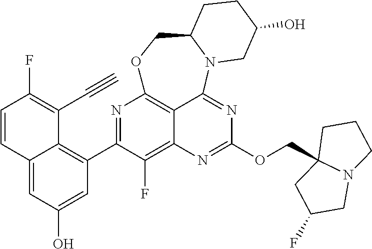 [SMILES: C#Cc1c(F)ccc2cc(O)cc(-c3nc4c5c(nc(OC[C@@]67CCCN6C[C@H](F)C7)nc5c3F)N3C[C@@H](O)CC[C@@H]3CO4)c12] | (8aR,11S)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-11-ol |
| 95 | 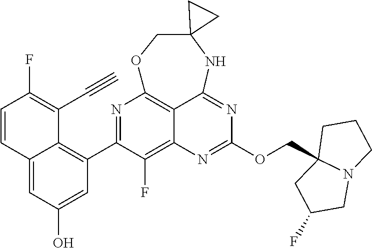 [SMILES: C#Cc1c(F)ccc2cc(O)cc(-c3nc4c5c(nc(OC[C@@]67CCCN6C[C@H](F)C7)nc5c3F)NC3(CC3)CO4)c12] | 5-ethynyl-6-fluoro-4-(4′-fluoro-2′- {[(2R,7aS)-2-fluorotetrahydro-1H- pyrrolizin-7a(5H)-yl]methoxy}- 8′H,10′H-spiro[cyclopropane-1,9′- [7]oxa[1,3,6,10]tetraazacyclohepta[1, 2,3-de]naphthalen]-5′-yl)naphthalen- 2-ol |
| 96 | 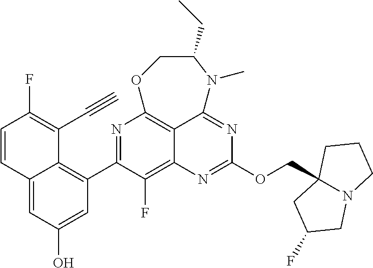 [SMILES: C#Cc1c(F)ccc2cc(O)cc(-c3nc4c5c(nc(OC[C@@]67CCCN6C[C@H](F)C7)nc5c3F)N(C)[C@@H](CC)CO4)c12] | 4-[(9S)-9-ethyl-4-fluoro-2- {[(2R,7aS)-2-fluorotetrahydro-1H- pyrrolizin-7a(5H)-yl]methoxy}-10- methyl-9,10-dihydro-8H-7-oxa- 1,3,6,10-tetraazacyclohepta[1,2,3- de]naphthalen-5-yl]-5-ethynyl-6- fluoronaphthalen-2-ol |
| 97 | 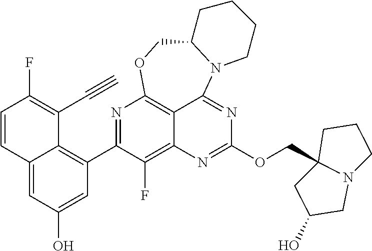 [SMILES: C#Cc1c(F)ccc2cc(O)cc(-c3nc4c5c(nc(OC[C@@]67CCCN6C[C@H](O)C7)nc5c3F)N3CCCC[C@H]3CO4)c12] | (2R,7aS)-7a-({[(8aS)-5-(8-ethynyl-7- fluoro-3-hydroxynaphthalen-1-yl)-4- fluoro-8,8a,9,10,11,12-hexahydro-7- oxa-1,3,6,12a- tetraazabenzo[4,5]cyclohepta[1,2,3- de]naphthalen-2- yl]oxy}methyl)hexahydro-1H- pyrrolizin-2-ol |
| 98 | 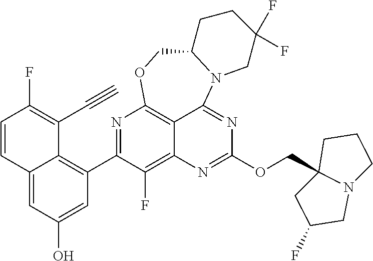 [SMILES: C#Cc1c(F)ccc2cc(O)cc(-c3nc4c5c(nc(OC[C@@]67CCCN6C[C@H](F)C7)nc5c3F)N3CC(F)(F)CC[C@H]3CO4)c12] | 5-ethynyl-6-fluoro-4-[(8aS)-4,11,11- trifluoro-2-{[(2R,7aS)-2- fluorotetrahydro-1H-pyrrolizin- 7a(5H)-yl]methoxy}-8,8a,9,10,11,12- hexahydro-7-oxa-1,3,6,12a- tetraazabenzo[4,5]cyclohepta[1,2,3- de]naphthalen-5-yl]naphthalen-2-ol |
| 99 | 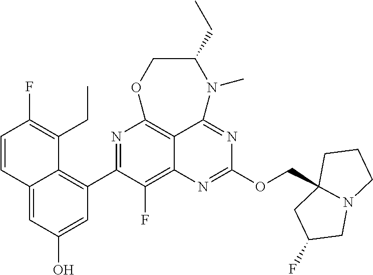 [SMILES: CCc1c(F)ccc2cc(O)cc(-c3nc4c5c(nc(OC[C@@]67CCCN6C[C@H](F)C7)nc5c3F)N(C)[C@@H](CC)CO4)c12] | 5-ethyl-4-[(9S)-9-ethyl-4-fluoro-2- {[(2R,7aS)-2-fluorotetrahydro-1H- pyrrolizin-7a(5H)-yl]methoxy}-10- methyl-9,10-dihydro-8H-7-oxa- 1,3,6,10-tetraazacyclohepta[1,2,3- de\|naphthalen-5-yl\|-6- fluoronaphthalen-2-ol |
| 100 | 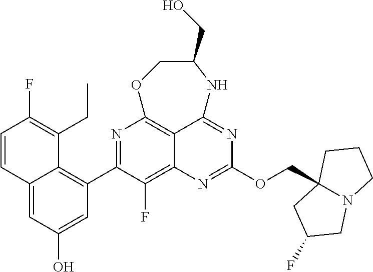 [SMILES: CCc1c(F)ccc2cc(O)cc(-c3nc4c5c(nc(OC[C@@]67CCCN6C[C@H](F)C7)nc5c3F)N[C@H](CO)CO4)c12] | 5-ethyl-6-fluoro-4-[(9R)-4-fluoro-2- {[(2R,7aS)-2-fluorotetrahydro-1H- pyrrolizin-7a(5H)-yl]methoxy}-9- (hydroxymethyl)-9,10-dihydro-8H-7- oxa-1,3,6,10- tetraazacyclohepta[1,2,3- de]naphthalen-5-yl]naphthalen-2-ol |
| 101 | 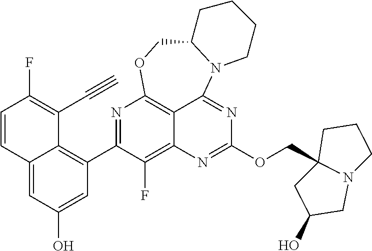 [SMILES: C#Cc1c(F)ccc2cc(O)cc(-c3nc4c5c(nc(OC[C@@]67CCCN6C[C@@H](O)C7)nc5c3F)N3CCCC[C@H]3CO4)c12] | (2S,7aS)-7a-({[(8aS)-5-(8-ethynyl-7- fluoro-3-hydroxynaphthalen-1-yl)-4- fluoro-8,8a,9,10,11,12-hexahydro-7- oxa-1,3,6,12a- tetraazabenzo[4,5]cyclohepta[1,2,3- de]naphthalen-2- yl]oxy}methyl)hexahydro-1H- pyrrolizin-2-ol |
| 102 | 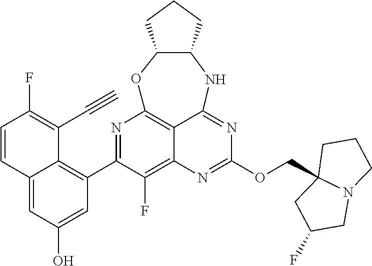 [SMILES: C#Cc1c(F)ccc2cc(O)cc(-c3nc4c5c(nc(OC[C@@]67CCCN6C[C@H](F)C7)nc5c3F)N[C@H]3CCC[C@H]3O4)c12] | 5-ethynyl-6-fluoro-4-[(7aR,10aS)-4- fluoro-2-{[(2R,7aS)-2- fluorotetrahydro-1H-pyrrolizin- 7a(5H)-yl]methoxy}- 7a,8,9,10,10a,11-hexahydro-7-oxa- 1,3,6,11-tetraazanaphtho[1,8- fg]azulen-5-yl]naphthalen-2-ol |
| 103 | 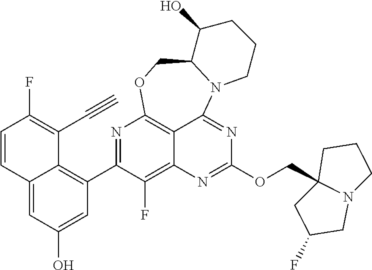 [SMILES: C#Cc1c(F)ccc2cc(O)cc(-c3nc4c5c(nc(OC[C@@]67CCCN6C[C@H](F)C7)nc5c3F)N3CCC[C@H](O)[C@@H]3CO4)c12] | (8aS,9S)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-9-ol |
| 104 | 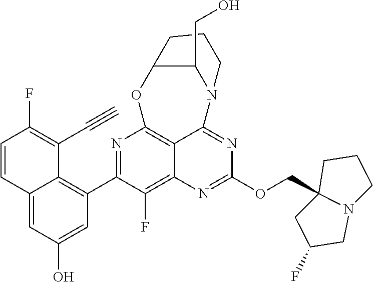 [SMILES: C#Cc1c(F)ccc2cc(O)cc(-c3nc4c5c(nc(OC[C@@]67CCCN6C[C@H](F)C7)nc5c3F)N3CCCC(O4)C3CO)c12] | 5-ethynyl-6-fluoro-4-[4-fluoro-2- {[(2R,7aS)-2-fluorotetrahydro-1H- pyrrolizin-7a(5H)-yl]methoxy}-13- (hydroxymethyl)-8,9,10,11- tetrahydro-8,12-methano-7-oxa- 1,3,6,12-tetraazacyclonona[1,2,3- de]naphthalen-5-yl]naphthalen-2-ol |
| 105 | 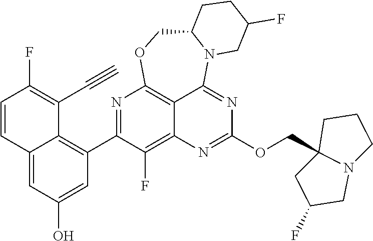 [SMILES: C#Cc1c(F)ccc2cc(O)cc(-c3nc4c5c(nc(OC[C@@]67CCCN6C[C@H](F)C7)nc5c3F)N3CC(F)CC[C@H]3CO4)c12] | 4-[(8aS)-4,11-difluoro-2-{[(2R,7aS)- 2-fluorotetrahydro-1H-pyrrolizin- 7a(5H)-yl]methoxy}-8,8a,9,10,11,12- hexahydro-7-oxa-1,3,6,12a- tetraazabenzo[4,5]cyclohepta[1,2,3- de]naphthalen-5-yl]-5-ethynyl-6- fluoronaphthalen-2-ol |
| 106 | 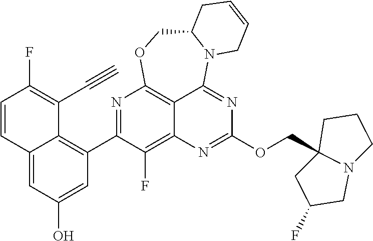 [SMILES: C#Cc1c(F)ccc2cc(O)cc(-c3nc4c5c(nc(OC[C@@]67CCCN6C[C@H](F)C7)nc5c3F)N3CC=CC[C@H]3CO4)c12] | 5-ethynyl-6-fluoro-4-[(8aS)-4-fluoro- 2-{[(2R,7aS)-2-fluorotetrahydro-1H- pyrrolizin-7a(5H)-yl]methoxy}- 8,8a,9,12-tetrahydro-7-oxa-1,3,6,12a- tetraazabenzo[4,5]cyclohepta[1,2,3- de]naphthalen-5-yl]naphthalen-2-ol |
| 107 | 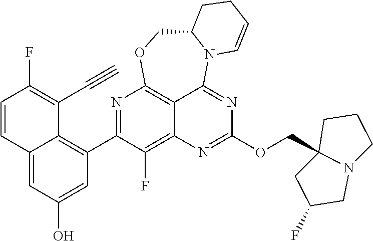 [SMILES: C#Cc1c(F)ccc2cc(O)cc(-c3nc4c5c(nc(OC[C@@]67CCCN6C[C@H](F)C7)nc5c3F)N3C=CCC[C@H]3CO4)c12] | 5-ethynyl-6-fluoro-4-[(8aS)-4-fluoro- 2-{[(2R,7aS)-2-fluorotetrahydro-1H- pyrrolizin-7a(5H)-yl]methoxy}- 8,8a,9,10-tetrahydro-7-oxa-1,3,6,12a- tetraazabenzo[4,5]cyclohepta[1,2,3- de]naphthalen-5-yl]naphthalen-2-ol |
| 108 | 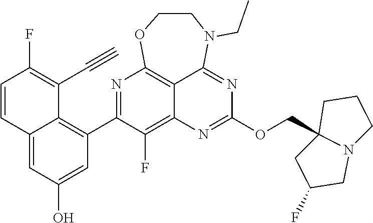 [SMILES: C#Cc1c(F)ccc2cc(O)cc(-c3nc4c5c(nc(OC[C@@]67CCCN6C[C@H](F)C7)nc5c3F)N(CC)CCO4)c12] | 4-(10-ethyl-4-fluoro-2-{ [(2R,7aS)-2- fluorotetrahydro-1H-pyrrolizin- 7a(5H)-yl]methoxy}-9,10-dihydro- 8H-7-oxa-1,3,6,10- tetraazacyclohepta[1,2,3- de]naphthalen-5-yl)-5-ethynyl-6- fluoronaphthalen-2-ol |
| 109 | 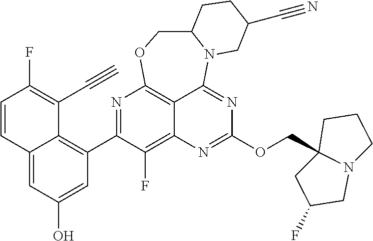 [SMILES: C#Cc1c(F)ccc2cc(O)cc(-c3nc4c5c(nc(OC[C@@]67CCCN6C[C@H](F)C7)nc5c3F)N3CC(C#N)CCC3CO4)c12] | 5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalene-11-carbonitrile |
| 110 | 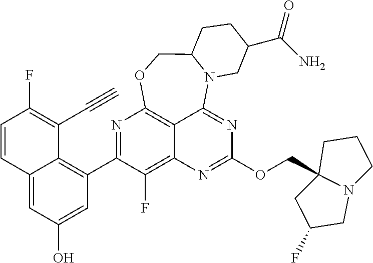 [SMILES: C#Cc1c(F)ccc2cc(O)cc(-c3nc4c5c(nc(OC[C@@]67CCCN6C[C@H](F)C7)nc5c3F)N3CC(C(N)=O)CCC3CO4)c12] | 5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalene-11-carboxamide |
| 111 | 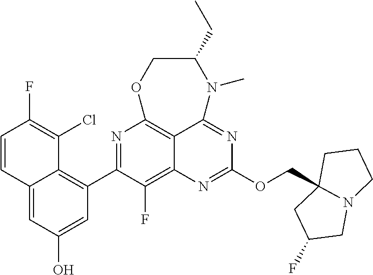 [SMILES: CC[C@H]1COc2nc(-c3cc(O)cc4ccc(F)c(Cl)c34)c(F)c3nc(OC[C@@]45CCCN4C[C@H](F)C5)nc(c23)N1C] | 5-chloro-4-[(9S)-9-ethyl-4-fluoro-2- {[(2R,7aS)-2-fluorotetrahydro-1H- pyrrolizin-7a(5H)-yl]methoxy}-10- methyl-9,10-dihydro-8H-7-oxa- 1,3,6,10-tetraazacyclohepta[1,2,3- de]naphthalen-5-yl]-6- fluoronaphthalen-2-ol |
| 112 | 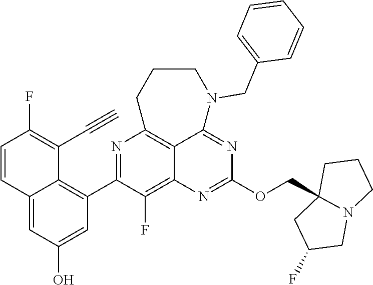 [SMILES: C#Cc1c(F)ccc2cc(O)cc(-c3nc4c5c(nc(OC[C@@]67CCCN6C[C@H](F)C7)nc5c3F)N(Cc3ccccc3)CCC4)c12] | 4-(10-benzyl-4-fluoro-2-{[(2R,7aS)- 2-fluorotetrahydro-1H-pyrrolizin- 7a(5H)-yl]methoxy}-7,8,9,10- tetrahydro-1,3,6,10- tetraazacyclohepta[1,2,3- de]naphthalen-5-yl)-5-ethynyl-6- fluoronaphthalen-2-ol |
| 113 | 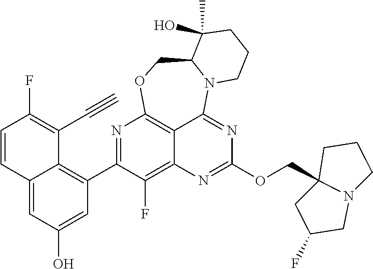 [SMILES: C#Cc1c(F)ccc2cc(O)cc(-c3nc4c5c(nc(OC[C@@]67CCCN6C[C@H](F)C7)nc5c3F)N3CCC[C@](C)(O)[C@@H]3CO4)c12] | (8aS,9S)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}-9- methyl-8,8a,9,10,11,12-hexahydro-7- oxa-1,3,6,12a- tetraazabenzo[4,5]cyclohepta[1,2,3- de]naphthalen-9-ol |
| 114 | 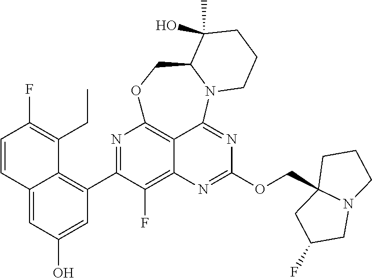 [SMILES: CCc1c(F)ccc2cc(O)cc(-c3nc4c5c(nc(OC[C@@]67CCCN6C[C@H](F)C7)nc5c3F)N3CCC[C@](C)(O)[C@@H]3CO4)c12] | (8aS,9S)-5-(8-ethyl-7-fluoro-3- hydroxynaphthalen-1-yl)-4-fluoro-2- {[(2R,7aS)-2-fluorotetrahydro-1H- pyrrolizin-7a(5H)-yl]methoxy}-9- methyl-8,8a,9,10,11,12-hexahydro-7- oxa-1,3,6,12a- tetraazabenzo[4,5]cyclohepta[1,2,3- de]naphthalen-9-ol |
| 115 | 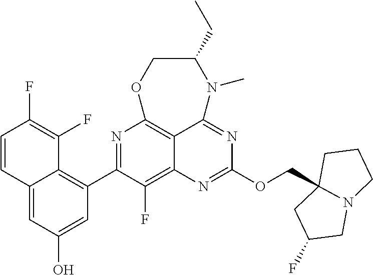 [SMILES: CC[C@H]1COc2nc(-c3cc(O)cc4ccc(F)c(F)c34)c(F)c3nc(OC[C@@]45CCCN4C[C@H](F)C5)nc(c23)N1C] | 4-[(9S)-9-ethyl-4-fluoro-2- {[(2R,7aS)-2-fluorotetrahydro-1H- pyrrolizin-7a(5H)-yl]methoxy}-10- methyl-9,10-dihydro-8H-7-oxa- 1,3,6,10-tetraazacyclohepta[1,2,3- de]naphthalen-5-yl]-5,6- difluoronaphthalen-2-ol |
| 116 | 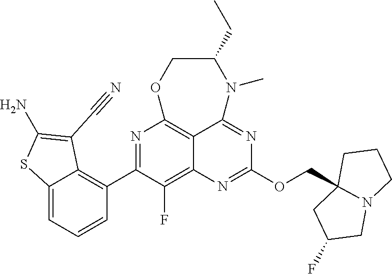 [SMILES: CC[C@H]1COc2nc(-c3cccc4sc(N)c(C#N)c34)c(F)c3nc(OC[C@@]45CCCN4C[C@H](F)C5)nc(c23)N1C] | 2-amino-4-[(9S)-9-ethyl-4-fluoro-2- {[(2R,7aS)-2-fluorotetrahydro-1H- pyrrolizin-7a(5H)-yl]methoxy}-10- methyl-9,10-dihydro-8H-7-oxa- 1,3,6,10-tetraazacyclohepta[1,2,3- de]naphthalen-5-yl]-1- benzothiophene-3-carbonitrile |
| 117 | 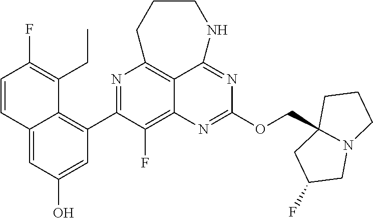 [SMILES: CCc1c(F)ccc2cc(O)cc(-c3nc4c5c(nc(OC[C@@]67CCCN6C[C@H](F)C7)nc5c3F)NCCC4)c12] | 5-ethyl-6-fluoro-4-(4-fluoro-2- {[(2R,7aS)-2-fluorotetrahydro-1H- pyrrolizin-7a(5H)-yl]methoxy}- 7,8,9,10-tetrahydro-1,3,6,10- tetraazacyclohepta[1,2,3- de]naphthalen-5-yl)naphthalen-2-ol |
| 118 | 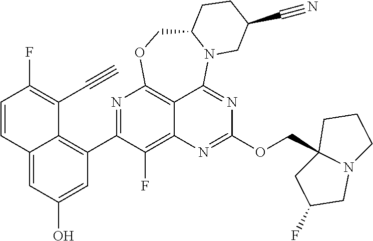 [SMILES: C#Cc1c(F)ccc2cc(O)cc(-c3nc4c5c(nc(OC[C@@]67CCCN6C[C@H](F)C7)nc5c3F)N3C[C@H](C#N)CC[C@H]3CO4)c12] | (5aS,8R)-2-(8-ethynyl-7-fluoro-3- hydroxynaphthalen-1-yl)-1-fluoro- 11-(((2R,7aS)-2-fluorotetrahydro-1H- pyrrolizin-7a(5H)-yl)methoxy)- 5,5a,6,7,8,9-hexahydro-4-oxa- 3,9a,10,12- tetraazabenzo[4,5]cyclohepta[1,2,3- de]naphthalene-8-carbonitrile |
| 119 | 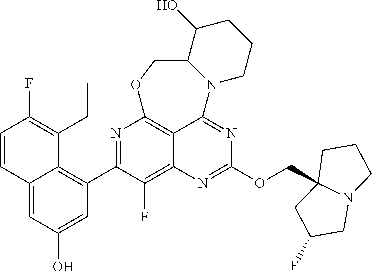 [SMILES: CCc1c(F)ccc2cc(O)cc(-c3nc4c5c(nc(OC[C@@]67CCCN6C[C@H](F)C7)nc5c3F)N3CCCC(O)C3CO4)c12] | 5-(8-ethyl-7-fluoro-3- hydroxynaphthalen-1-yl)-4-fluoro-2- {[(2R,7aS)-2-fluorotetrahydro-1H- pyrrolizin-7a(5H)-yl]methoxy}- 8,8a,9,10,11,12-hexahydro-7-oxa- 1,3,6,12a- tetraazabenzo[4,5]cyclohepta[1,2,3- de]naphthalen-9-ol |
| 120 | 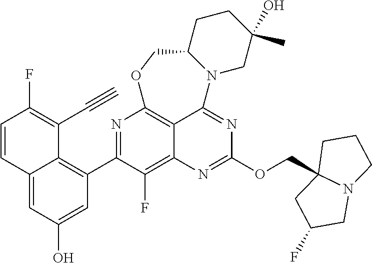 [SMILES: C#Cc1c(F)ccc2cc(O)cc(-c3nc4c5c(nc(OC[C@@]67CCCN6C[C@H](F)C7)nc5c3F)N3C[C@@](C)(O)CC[C@H]3CO4)c12] | (8aS,11S)-5-(8-ethynyl-7-fluoro-3- hydroxynaphthalen-1-yl)-4-fluoro-2- {[(2R,7aS)-2-fluorotetrahydro-1H- pyrrolizin-7a(5H)-yl]methoxy}-11- methyl-8,8a,9,10,11,12-hexahydro-7- oxa-1,3,6,12a- tetraazabenzo[4,5]cyclohepta[1,2,3- de]naphthalen-11-ol |
| 121 | 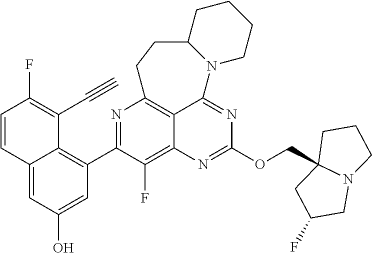 [SMILES: C#Cc1c(F)ccc2cc(O)cc(-c3nc4c5c(nc(OC[C@@]67CCCN6C[C@H](F)C7)nc5c3F)N3CCCCC3CC4)c12] | 5-ethynyl-6-fluoro-4-(4-fluoro-2- {[(2R,7aS)-2-fluorotetrahydro-1H- pyrrolizin-7a(5H)-yl]methoxy}- 8,8a,9,10,11,12-hexahydro-7H- 1,3,6,12a- tetraazabenzo[4,5]cyclohepta[1,2,3- de]naphthalen-5-yl)naphthalen-2-ol |
| 122 | 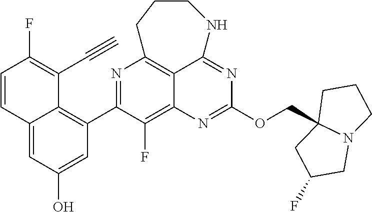 [SMILES: C#Cc1c(F)ccc2cc(O)cc(-c3nc4c5c(nc(OC[C@@]67CCCN6C[C@H](F)C7)nc5c3F)NCCC4)c12] | 5-ethynyl-6-fluoro-4-(4-fluoro-2- {[(2R,7aS)-2-fluorotetrahydro-1H- pyrrolizin-7a(5H)-yl]methoxy}- 7,8,9,10-tetrahydro-1,3,6,10- tetraazacyclohepta[1,2,3- de]naphthalen-5-yl)naphthalen-2-ol |
| 123 | 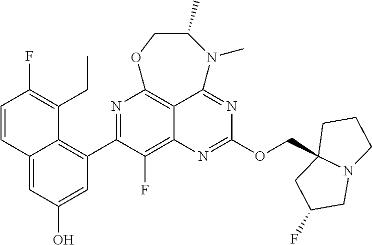 [SMILES: CCc1c(F)ccc2cc(O)cc(-c3nc4c5c(nc(OC[C@@]67CCCN6C[C@H](F)C7)nc5c3F)N(C)[C@@H](C)CO4)c12] | 5-ethyl-6-fluoro-4-[(9S)-4-fluoro-2- {[(2R,7aS)-2-fluorotetrahydro-1H- pyrrolizin-7a(5H)-yl]methoxy}-9,10- dimethyl-9,10-dihydro-8H-7-oxa- 1,3,6,10-tetraazacyclohepta[1,2,3- de]naphthalen-5-yl]naphthalen-2-ol |
| 124 | 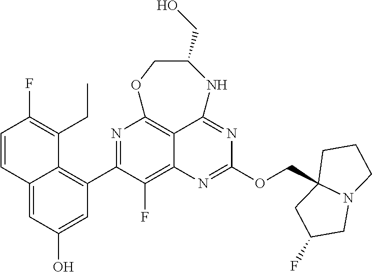 [SMILES: CCc1c(F)ccc2cc(O)cc(-c3nc4c5c(nc(OC[C@@]67CCCN6C[C@H](F)C7)nc5c3F)N[C@@H](CO)CO4)c12] | 5-ethyl-6-fluoro-4-[(9S)-4-fluoro-2- {[(2R,7aS)-2-fluorotetrahydro-1H- pyrrolizin-7a(5H)-yl]methoxy}-9- (hydroxymethyl)-9,10-dihydro-8H-7- oxa-1,3,6,10- tetraazacyclohepta[1,2,3- de]naphthalen-5-yl]naphthalen-2-ol |
| 125 | 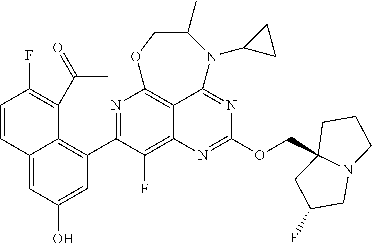 [SMILES: CC(=O)c1c(F)ccc2cc(O)cc(-c3nc4c5c(nc(OC[C@@]67CCCN6C[C@H](F)C7)nc5c3F)N(C3CC3)C(C)CO4)c12] | 1-[8-(10-cyclopropyl-4-fluoro-2- {[(2R,7aS)-2-fluorotetrahydro-1H- pyrrolizin-7a(5H)-yl]methoxy}-9- methyl-9,10-dihydro-8H-7-oxa- 1,3,6,10-tetraazacyclohepta[1,2,3- de]naphthalen-5-yl)-2-fluoro-6- hydroxynaphthalen-1-yl]ethan-1-one |
| 126 | 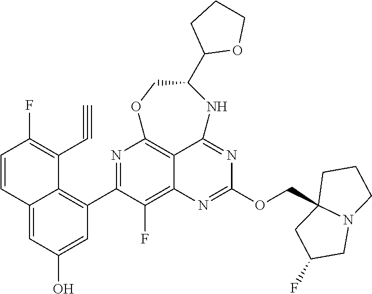 [SMILES: C#Cc1c(F)ccc2cc(O)cc(-c3nc4c5c(nc(OC[C@@]67CCCN6C[C@H](F)C7)nc5c3F)N[C@@H](C3CCCO3)CO4)c12] | 5-ethynyl-6-fluoro-4-((9R)-4-fluoro- 2-(((2R,7aS)-2-fluorotetrahydro-1H- pyrrolizin-7a(5H)-yl)methoxy)-9- (tetrahydrofuran-2-yl)-9,10-dihydro- 8H-7-oxa-1,3,6,10- tetraazacyclohepta[de]naphthalen-5- yl)naphthalen-2-ol |
| 127 | 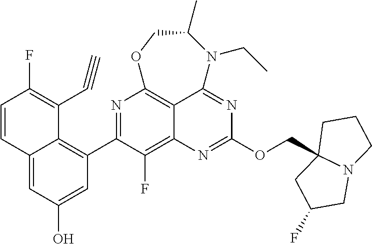 [SMILES: C#Cc1c(F)ccc2cc(O)cc(-c3nc4c5c(nc(OC[C@@]67CCCN6C[C@H](F)C7)nc5c3F)N(CC)[C@@H](C)CO4)c12] | 4-((S)-10-ethyl-4-fluoro-2- (((2R,7aS)-2-fluorotetrahydro-1H- pyrrolizin-7a(5H)-yl)methoxy)-9- methyl-9,10-dihydro-8H-7-oxa- 1,3,6,10- tetraazacyclohepta[de]naphthalen-5- yl)-5-ethynyl-6-fluoronaphthalen-2- ol |
| 128 | 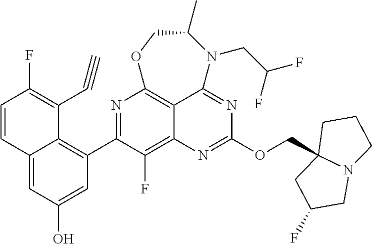 [SMILES: C#Cc1c(F)ccc2cc(O)cc(-c3nc4c5c(nc(OC[C@@]67CCCN6C[C@H](F)C7)nc5c3F)N(CC(F)F)[C@@H](C)CO4)c12] | 4-((S)-10-(2,2-difluoroethyl)-4- fluoro-2-(((2R,7aS)-2- fluorotetrahydro-1H-pyrrolizin- 7a(5H)-yl)methoxy)-9-methyl-9,10- dihydro-8H-7-oxa-1,3,6,10- tetraazacyclohepta[de]naphthalen-5- yl)-5-ethynyl-6-fluoronaphthalen-2- ol |
| 129 | 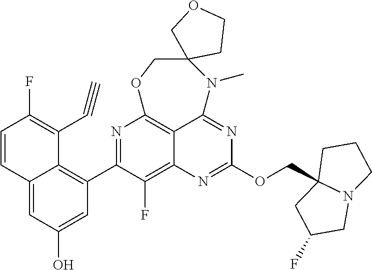 [SMILES: C#Cc1c(F)ccc2cc(O)cc(-c3nc4c5c(nc(OC[C@@]67CCCN6C[C@H](F)C7)nc5c3F)N(C)C3(CCOC3)CO4)c12] | 5-ethynyl-6-fluoro-4-(4-fluoro-2- (((2R,7aS)-2-fluorotetrahydro-1H- pyrrolizin-7a(5H)-yl)methoxy)-10- methyl-4′,5′-dihydro-2′H,8H,10H-7- oxa-1,3,6,10- tetraazaspiro[cyclohepta[de]naphthalene- 9,3′-furan]-1(10a),2,3a,3a1(6a),5- pentaen-5-yl)naphthalen-2-ol |
| 130 | 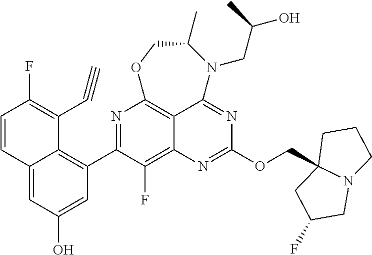 [SMILES: C#Cc1c(F)ccc2cc(O)cc(-c3nc4c5c(nc(OC[C@@]67CCCN6C[C@H](F)C7)nc5c3F)N(C[C@@H](C)O)[C@@H](C)CO4)c12] | 5-ethynyl-6-fluoro-4-((S)-4-fluoro-2- (((2R,7aS)-2-fluorotetrahydro-1H- pyrrolizin-7a(5H)-yl)methoxy)-10- ((R)-2-hydroxypropyl)-9-methyl- 9,10-dihydro-8H-7-oxa-1,3,6,10- tetraazacyclohepta[de]naphthalen-5- yl)naphthalen-2-ol |
| 131 | 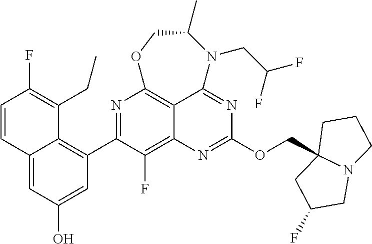 [SMILES: CCc1c(F)ccc2cc(O)cc(-c3nc4c5c(nc(OC[C@@]67CCCN6C[C@H](F)C7)nc5c3F)N(CC(F)F)[C@@H](C)CO4)c12] | 4-((S)-10-(2,2-difluoroethyl)-4- fluoro-2-(((2R,7aS)-2- fluorotetrahydro-1H-pyrrolizin- 7a(5H)-yl)methoxy)-9-methyl-9,10- dihydro-8H-7-oxa-1,3,6,10- tetraazacyclohepta[de]naphthalen-5- yl)-5-ethyl-6-fluoronaphthalen-2-ol |
| 132 | 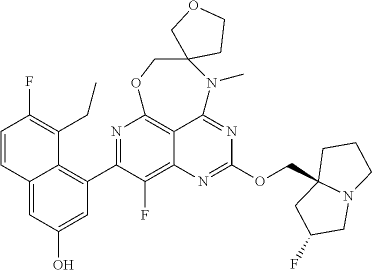 [SMILES: CCc1c(F)ccc2cc(O)cc(-c3nc4c5c(nc(OC[C@@]67CCCN6C[C@H](F)C7)nc5c3F)N(C)C3(CCOC3)CO4)c12] | 5-ethyl-6-fluoro-4-(4-fluoro-2- (((2R,7aS)-2-fluorotetrahydro-1H- pyrrolizin-7a(5H)-yl)methoxy)-10- methyl-4′,5′-dihydro-2′H,8H,10H-7- oxa-1,3,6,10- tetraazaspiro[cyclohepta[de]naphthalene- 9,3′-furan]-1(10a),2,3a,3a1(6a),5- pentaen-5-yl)naphthalen-2-ol |
| 133 | 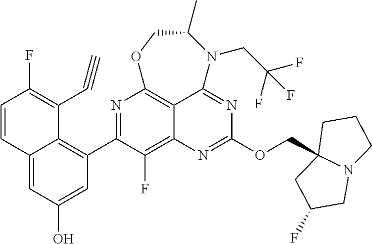 [SMILES: C#Cc1c(F)ccc2cc(O)cc(-c3nc4c5c(nc(OC[C@@]67CCCN6C[C@H](F)C7)nc5c3F)N(CC(F)(F)F)[C@@H](C)CO4)c12] | 5-ethynyl-6-fluoro-4-((S)-4-fluoro-2- (((2R,7aS)-2-fluorotetrahydro-1H- pyrrolizin-7a(5H)-yl)methoxy)-9- methyl-10-(2,2,2-trifluoroethyl)-9,10- dihydro-8H-7-oxa-1,3,6,10- tetraazacyclohepta[de]naphthalen-5- yl)naphthalen-2-ol |

and pharmaceutically acceptable salts thereof.

Those skilled in the art will recognize that the species listed or illustrated herein are not exhaustive, and that additional species within the scope of these defined terms may also be selected.

### Pharmaceutical Compositions

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the disclosure are sterile compositions. Pharmaceutical compositions may be prepared using compounding techniques known or that become available to those skilled in the art.

Sterile compositions are also contemplated by the disclosure, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the disclosure may be administered by a suitable route of delivery, such as oral, parenteral, rectal, nasal, topical, or ocular routes, or by inhalation. Preferably, the compositions are formulated for intravenous or oral administration.

For oral administration, the compounds the disclosure may be provided in a solid form, such as a tablet or capsule, or as a solution, emulsion, or suspension. To prepare the oral compositions, the compounds of the disclosure may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 g/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present disclosure are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the disclosure may utilize a patch formulation to effect transdermal delivery.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human.

Exemplary diseases include cancer, pain, neurological diseases, autoimmune diseases, and inflammation. As used herein, the term “cancer” includes, but is not limited to, ALCL, NSCLC, neuroblastoma, inflammatory myofibroblastic tumor, adult renal cell carcinoma, pediatric renal cell carcinoma, breast cancer, ER⁺ breast cancer, colonic adenocarcinoma, glioblastoma, glioblastoma multiforme, anaplastic thyroid cancer, cholangiocarcinoma, ovarian cancer, gastric adenocarcinoma, colorectal cancer, inflammatory myofibroblastic tumor, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cutaneous melanoma, head and neck squamous cell carcinoma, pediatric glioma CML, prostate cancer, lung squamous carcinoma, ovarian serous cystadenocarcinoma, skin cutaneous melanoma, castrate-resistant prostate cancer, Hodgkin lymphoma, and serous and clear cell endometrial cancer. In some embodiments, cancer includes lung cancer, colon cancer, breast cancer, prostate cancer, hepatocellular carcinoma, renal cell carcinoma, gastric and esophago-gastric cancers, glioblastoma, head and neck cancers, inflammatory myofibroblastic tumors, and anaplastic large cell lymphoma. Pain includes, for example, pain from any source or etiology, including cancer pain, pain from chemotherapeutic treatment, nerve pain, pain from injury, or other sources. Autoimmune diseases include, for example, rheumatoid arthritis, Sjogren syndrome, Type I diabetes, and lupus. Exemplary neurological diseases include Alzheimer's Disease, Parkinson's Disease, Amyotrophic lateral sclerosis, and Huntington's disease. Exemplary inflammatory diseases include atherosclerosis, allergy, and inflammation from infection or injury.

In one aspect, the compounds and pharmaceutical compositions of the disclosure specifically target Ras, in particular K-Ras. Thus, these compounds and pharmaceutical compositions can be used to prevent, reverse, slow, or inhibit the activity of one or more KRAS mutations, such as KRAS G12C, KRAS G12D, KRAS G12V, KRAS G12R, KRAS G12S, KRAS G13C, KRAS G13D, KRAS A18D, KRAS Q61H, KRAS K117N, and the like. In preferred embodiments, methods of treating a target cancer are described.

In the inhibitory methods of the disclosure, an “effective amount” means an amount sufficient to inhibit the target protein. Measuring such target modulation may be performed by routine analytical methods such as those described below. Such modulation is useful in a variety of settings, including in vitro assays. In such methods, the cell is preferably a cancer cell with abnormal signaling due to a mutation of KRAS, such as KRAS G12C, KRAS G12D, KRAS G12V, KRAS G12R, KRAS G12S, KRAS G13C, KRAS G13D, KRAS A18D, KRAS Q61H, KRAS K117N, and the like.

In treatment methods according to the disclosure, an “effective amount” means an amount or dose sufficient to generally bring about the desired therapeutic benefit in subjects needing such treatment. Effective amounts or doses of the compounds of the disclosure may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### Drug Combinations

The inventive compounds described herein may be used in pharmaceutical compositions or methods in combination with one or more additional active ingredients in the treatment of the diseases and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present disclosure or may be included with a compound of the present disclosure in a single pharmaceutical composition. The additional active ingredients may be administered simultaneously with, prior to, or after administration of a compound of the present disclosure.

Combination agents include additional active ingredients are those that are known or discovered to be effective in treating the diseases and disorders described herein, including those active against another target associated with the disease. For example, compositions and formulations of the disclosure, as well as methods of treatment, can further comprise other drugs or pharmaceuticals, e.g., other active agents useful for treating or palliative for the target diseases or related symptoms or conditions. For cancer indications, additional such agents include, but are not limited to, kinase inhibitors, such as ALK inhibitors (e.g. crizotinib), Raf inhibitors (e.g., vemurafenib), VEGFR inhibitors (e.g., sunitinib), standard chemotherapy agents such as alkylating agents, antimetabolites, anti-tumor antibiotics, topoisomerase inhibitors, platinum drugs, mitotic inhibitors, antibodies, hormone therapies, or corticosteroids. For pain indications, suitable combination agents include anti-inflammatories such as NSAIDs. The pharmaceutical compositions of the disclosure may additional comprise one or more of such active agents, and methods of treatment may additionally comprise administering an effective amount of one or more of such active agents.

### Chemical Synthesis Methods

The following examples are offered to illustrate but not to limit the disclosure. One of skill in the art will recognize that the following synthetic reactions and schemes may be modified by choice of suitable starting materials and reagents in order to access other compounds of Formula (I)—(XXXXVI).

Abbreviations: The examples described herein use materials, including but not limited to, those described by the following abbreviations known to those skilled in the art:

| | |
|---|---|
| g | grams |
| eq | equivalents |
| mmol | millimoles |
| mL | milliliters |
| EtOAc | ethyl acetate |
| MHz | megahertz |
| ppm | parts per million |
| δ | chemical shift |
| s | singlet |
| d | doublet |
| t | triplet |
| q | quartet |
| quin | quintet |
| br | broad |
| m | multiplet |
| Hz | hertz |
| THF | tetrahydrofuran |
| ° C. | degrees Celsius |
| PE | petroleum ether |
| EA | ethyl acetate |
| R_{f} | retardation factor |
| N | normal |
| J | coupling constant |
| DMSO-d₆ | deuterated dimethyl sulfoxide |
| n-BuOH | n-butanol |
| DIEA | n,n-diisopropylethylamine |
| TMSCl | trimethylsilyl chloride |
| min | minutes |
| hr | hours |
| Me | methyl |
| Et | ethyl |
| i-Pr | isopropyl |
| TLC | thin layer chromatography |
| M | molar |
| Compd# | compound number |
| MS | mass spectrum |
| m/z | mass-to-charge ratio |
| Ms | methanesulfonyl |
| FDPP | pentafluorophenyl diphenylphosphinate |
| Boc | tert-butyloxycarbonyl |
| TFA | trifluoroacetic acid |
| Tos | toluenesulfonyl |
| DMAP | 4-(dimethylamino)pyridine |
| mM | micromolar |
| ATP | adenosine triphosphate |
| IC₅₀ | half maximal inhibitory concentration |
| U/mL | units of activity per milliliter |
| KHMDS | potassium bis(trimethylsilyl)amide |
| DIAD | diisopropyl azodicarboxylate |
| MeTHF | 2-methyltetrahydrofuran |
| MOM | methoxymethyl |
| DCM | dichloromethane |
| DMF | N,N-dimethylformamide |
| DPPA | diphenyl phosphoryl azide |
| DBU | 1,8-diazabicyclo[5.4.0]undec-7-ene |
| DIPEA | N,N-diisopropylethylamine |
| SEM | [2-(Trimethylsilyl)ethoxy]methyl acetal |
| Hex | hexanes |
| Pd(dppf)Cl₂ | [1,1′-Bis(diphenylphosphino)ferrocene]dichloropalladium(II) |
| MeCN (ACN) | Acetonitrile |
| Pd₂(dba)₃ | Tris(dibenzylideneacetone)dipalladium(0) |
| Hunig's Base | N,N-diisopropylethylamine |
| TBAF | Tert butyl ammonium fluoride |
| PPh₃ | Triphenyl phosphine |
| RT | Room Temperature |
| p-TSA | Para-Tolylsulfonic acid |
| t-BuOH | Tert-Butanol |
| Pd(amphos)Cl₂ | Dichlorobis[di-tert-butyl(4- |
| | dimethylaminophenyl)phosphine┘palladium(II) |
| mCPBA | Meta-Chloroperoxy benzoic acid |
| AcOH | Acetic Acid |
| DMAc | N,N-Dimethylformamide |
| BPD | 4,4,5,5-tetramethyl-2-(4,4,5,5-tetramethyl- |
| | 1,3,2-dioxaborolan-2-yl)-1,3,2-dioxaborolane |
| MTBE | Methyl tert-Butyl Ether |

The proposed targets can be prepared via the conventional chemistry or following the general schemes as shown below.

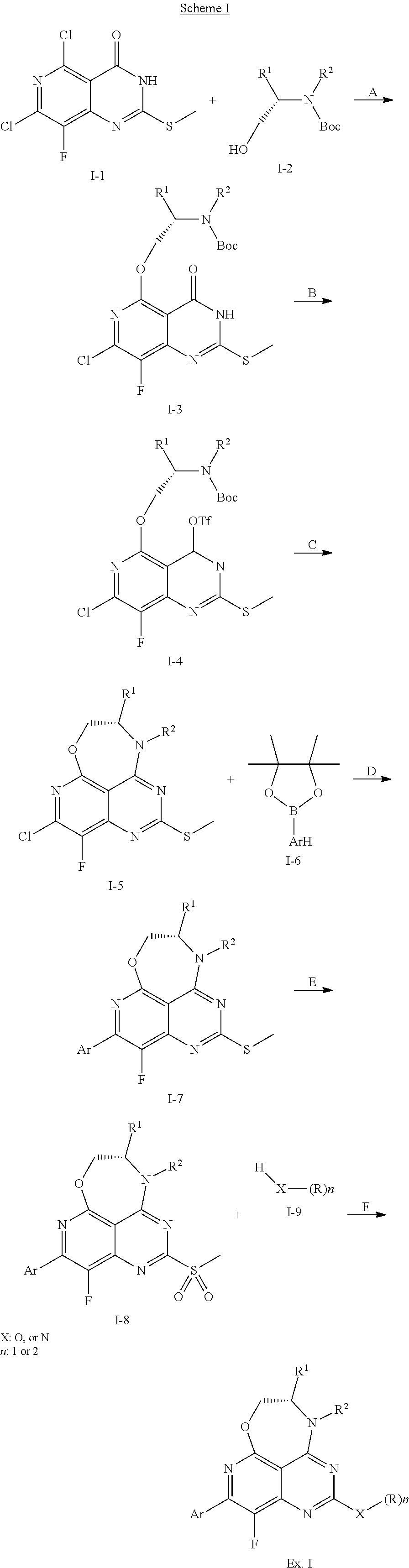

The general scheme I is used to prepare the products with general structure Ex. 1. The bicyclic aryl core I-1 and a variety of alcohol 1-2 are either commercially available or prepared via conventional chemistry from commercially available materials. A: at 0° C. in DMA as solvent, 1-2 is deprotonated with base such as NaH, then reaction with compound I-1 to give product 1-3. B: under basic conditions such as pyridine in DCM as solvent at 0° C., 1-3 is converted to product I-4. C: After Boc deprotection of 1-4 with acid such as TFA, the product is converted to product I-5 under basic conditions such as diisopropylethylamine in ACN at room temperature. D: under palladium-catalyzed Suzuki coupling condition, compound I-5 and a boronic ester I-6 are converted to a product 1-7. E: at room temperature in DCM as solvent, I-7 is converted to I-8 with mCPBA. E: under basic conditions such as diisopropylethylamine in dioxane at elevated temperature such as 80° C., compound I-8 and an amine or alcohol I-9 are converted to a product which is de-protected if is required to provide the final product Ex. I.

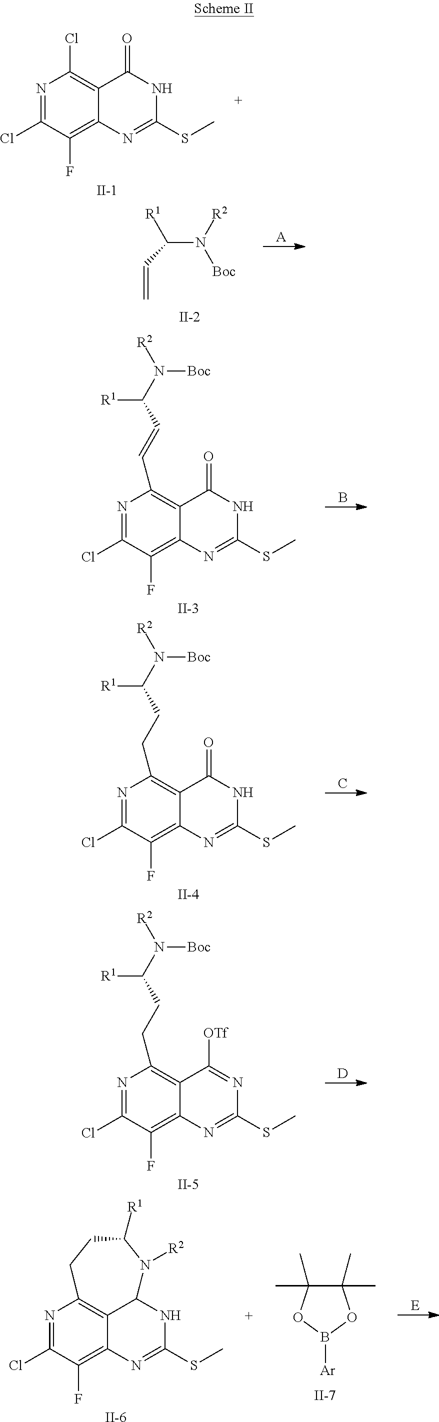

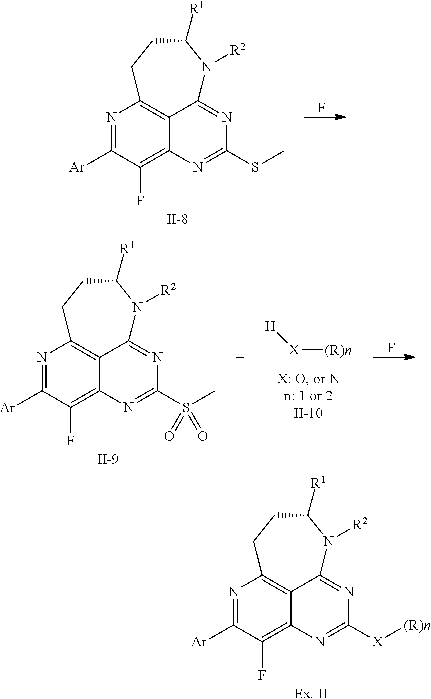

The general scheme II is used to prepare the products with general structure Ex. 4. The bicyclic aryl core II-1 and a variety of alkenes II-2 are either commercially available or prepared via conventional chemistry from commercially available materials. A: under palladium-catalyzed Heck coupling condition, compound II-1 and alkene II-2 are converted to a product II-3. B: under an atmosphere of hydrogen at room temperature in ethanol as solvent, compound II-3 are converted to a product II-4 with palladium on carbon. C: at low temperature, such as 0° C., in DCM as solvent, II-4 is converted to II-5 with trifluoromethanesulfonic anhydride and an organic base such as diisopropylethylamine. D: After Boc deprotection of II-5 with acid such as TFA, the product is converted to product II-6 under basic conditions such as diisopropylethylamine in DMF at elevated temperature. E: under palladium-catalyzed Suzuki coupling condition, compound II-6 and a boronic ester II-7 are converted to a product II-8. F: at room temperature in DCM as solvent, II-8 is converted to II-9 with mCPBA. G: under basic conditions such as diisopropylethylamine in dioxane at elevated temperature such as 80° C., compound II-9 and an amine or alcohol II-10 are converted to a product which is de-protected, if it is required, to provide the final product Ex. II.

Intermediate Table 1.

| Comp. # | Structure |
|---|---|
| 1-2-1 | 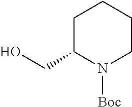 [SMILES: CC(C)(C)OC(=O)N1CCCC[C@H]1CO] |
| I-2-2 | 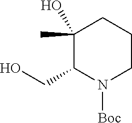 [SMILES: CC(C)(C)OC(=O)N1CCC[C@@](C)(O)[C@H]1CO] |
| II-2-1 | 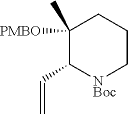 |
| I-2-3 | 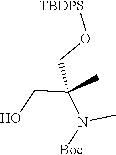 |
| II-2-2 | 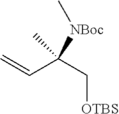 [SMILES: C=C[C@](C)(CO[Si](C)(C)C(C)(C)C)N(C)C(=O)OC(C)(C)C] |
| II-2-3 | 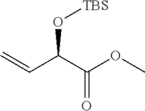 |
| II-2-4 | 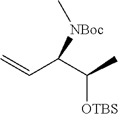 [SMILES: C=C[C@H]([C@@H](C)O[Si](C)(C)C(C)(C)C)N(C)C(=O)OC(C)(C)C] |
| II-2-5 | 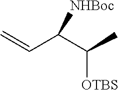 [SMILES: C=C[C@@H](NC(=O)OC(C)(C)C)[C@@H](C)O[Si](C)(C)C(C)(C)C] |
| II-2-6 | 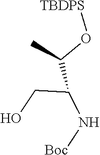 |
| II-2-7 | 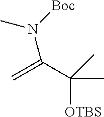 [SMILES: C=C(N(C)C(=O)OC(C)(C)C)C(C)(C)O[Si](C)(C)C(C)(C)C] |

I-2-1 is commercially available.

### Preparation of tert-butyl (2R,3R)-3-hydroxy-2-(hydroxymethyl)-3-methylpiperidine-1-carboxylate (I-2-2)

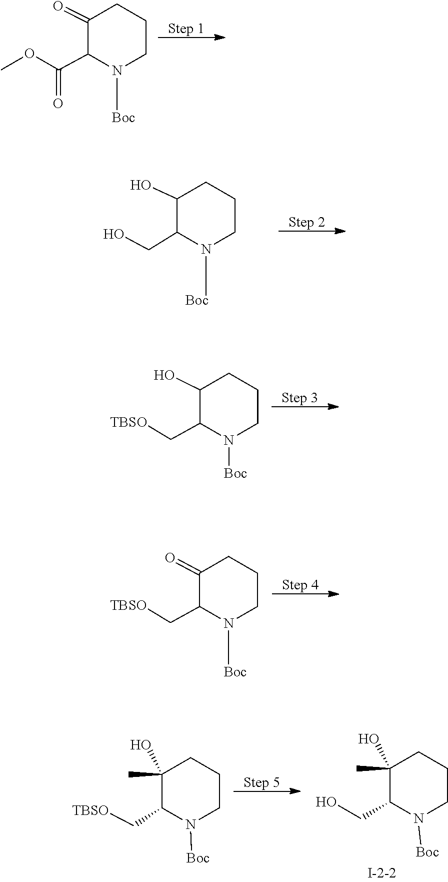 [SMILES: COC(=O)C1C(=O)CCCN1C(=O)OC(C)(C)C ; CC(C)(C)OC(=O)N1CCCC(O)C1CO ; CC(C)(C)OC(=O)N1CCCC(O)C1CO[Si](C)(C)C(C)(C)C ; CC(C)(C)OC(=O)N1CCCC(=O)C1CO[Si](C)(C)C(C)(C)C ; CC(C)(C)OC(=O)N1CCC[C@@](C)(O)[C@H]1CO[Si](C)(C)C(C)(C)C ; CC(C)(C)OC(=O)N1CCC[C@@](C)(O)[C@H]1CO]

Step 1. To the solution of 1-tert-butyl 2-methyl 3-oxopiperidine-1,2-dicarboxylate (1.0 eq.) in THE and methanol at room temperature is added lithium borohydride (4 eq.) and the mixture is stirred until reaction completion. Extraction workup with EtOAc followed by column chromatography affords tert-butyl 3-hydroxy-2-(hydroxymethyl)piperidine-1-carboxylate.

Step 2. To the mixture of tert-butyl 3-hydroxy-2-(hydroxymethyl)piperidine-1-carboxylate (1.0 eq.), imidazole ((4.0 eq.) in DMF is added TBS-Cl (1.0 eq.). The reaction mixture is stirred until reaction completion. Extraction workup with EtOAc followed by column chromatography affords product tert-butyl 2-({[tert-butyl(dimethyl)silyl]oxy}methyl)-3-hydroxypiperidine-1-carboxylate.

Step 3. To a solution of 2-({[tert-butyl(dimethyl)silyl]oxy}methyl)-3-hydroxypiperidine-1-carboxylate (1.0 eq.) in anhydrous DCM at 0° C. is added 1,1,1-tris(actyloxy)-1,1-dihydro-1,2-benzodioxol-3-(1H)-one (4.0 eq.). The mixture is allowed to stir at room temperature under nitrogen until reaction completion. Extraction workup with EtOAc followed by column chromatography affords tert-butyl 2-({[tert-butyl(dimethyl)silyl]oxy}methyl)-3-oxopiperidine-1-carboxylate.

Step 4. To the solution of methyl Grignard (1M in Et₂O, 4.0 eq.) at 0° C. under argon, is added the solution of tert-butyl 2-({[tert-butyl(dimethyl)silyl]oxy}methyl)-3-oxopiperidine-1-carboxylate (1.0 eq.) in THE slowly, and the reaction mixture is allowed to warm to room temperature and stirred until reaction completion. The reaction was then quenched with saturated aqueous NH₄Cl. Extraction workup followed by column chromatography with chiral column affords tert-butyl (2R,3R)-2-({[tert-butyl(dimethyl)silyl]oxy}methyl)-3-hydroxy-3-methylpiperidine-1-carboxylate.

Step 5. To the solution of tert-butyl (2R,3R)-2-({[tert-butyl(dimethyl)silyl]oxy}methyl)-3-hydroxy-3-methylpiperidine-1-carboxylate (1.0 eq.) in THF is added TBAF (2 eq.) and the mixture is stirred until reaction completion. Extraction workup with EtOAc followed by column chromatography affords intermediate I-2-2.

### Preparation of tert-butyl [(2R)-1-{[tert-butyl(diphenyl)silyl]oxy}-3-hydroxy-2-methylpropan-2-yl]methylcarbamate (I-2-3)

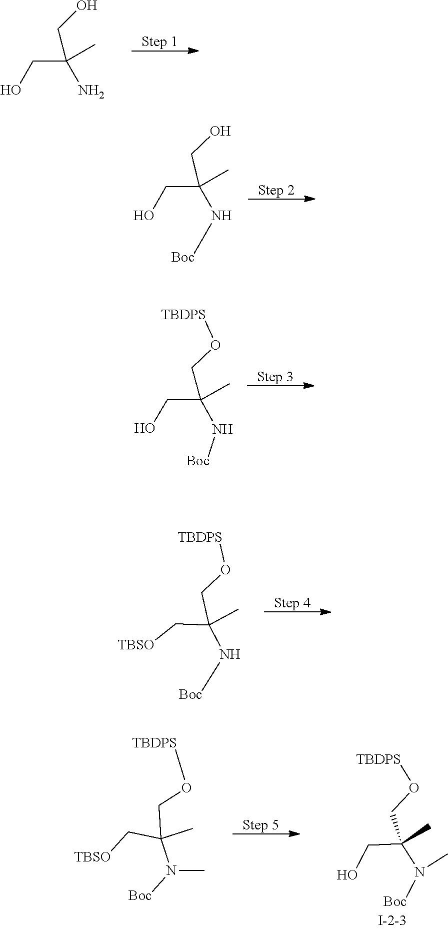

Step 1. To the solution of 2-Amino-2-methyl-1,3-propanediol (1.0 eq.) and DIEA (2.0 eq.) in DCM is added di-tert-butyl decarbonate (1.0 eq.) and mixture is stirred until reaction completion. Extraction workup with EtOAc followed by column chromatography affords ten-butyl (1,3-dihydroxy-2-methylpropan-2-yl)carbamate.

Step 2. To the mixture tert-butyl (1,3-dihydroxy-2-methylpropan-2-yl)carbamate (1.0 eq.), imidazole ((4.0 eq.) in DMF is added TBDPS-Cl (1.2 eq.). The reaction mixture is stirred until reaction completion. Extraction workup with EtOAc followed by column chromatography affords product tert-butyl (1-{[tert-butyl(diphenyl)silyl]oxy}-3-hydroxy-2-methylpropan-2-yl)carbamate.

Step 3. To the mixture of tert-butyl (1-{[tert-butyl(diphenyl)silyl]oxy}-3-hydroxy-2-methylpropan-2-yl)carbamate (1.0 eq.), imidazole ((4.0 eq.) in DMF is added TBS-C₁ (1.0 eq.). The reaction mixture is stirred until reaction completion. Extraction workup with EtOAc followed by column chromatography affords product tert-butyl (2,2,3,3,6,10,10-heptamethyl-9,9-diphenyl-4,8-dioxa-3,9-disilaundecan-6-yl)carbamate.

Step 4. To the solution of tert-butyl (2,2,3,3,6,10,10-heptamethyl-9,9-diphenyl-4,8-dioxa-3,9-disilaundecan-6-yl)carbamate (1.0 eq.) in THF is added NaH (1.2 eq.). After stirred for 20 minutes, Mel (1.4 eq.) is added, and the reaction mixture is stirred until reaction completion. After water is added to quench the reaction, extraction workup with EtOAc followed by column chromatography affords tert-butyl (2,2,3,3,6,10,10-heptamethyl-9,9-diphenyl-4,8-dioxa-3,9-disilaundecan-6-yl)methylcarbamate.

Step 5. To the solution of tert-butyl (2,2,3,3,6,10,10-heptamethyl-9,9-diphenyl-4,8-dioxa-3,9-disilaundecan-6-yl)methylcarbamate (1.0 eq.) in EtOH is added pyridinium p-toluenesulfonate (2 eq.) and the mixture is stirred until reaction completion. Extraction workup with EtOAc followed by column chromatography with chiral column affords intermediate I-2-3.

### Preparation of tert-butyl (2R,3R)-3-((4-methoxybenzyl)oxy)-3-methyl-2-vinylpiperidine-1-carboxylate (II-2-1)

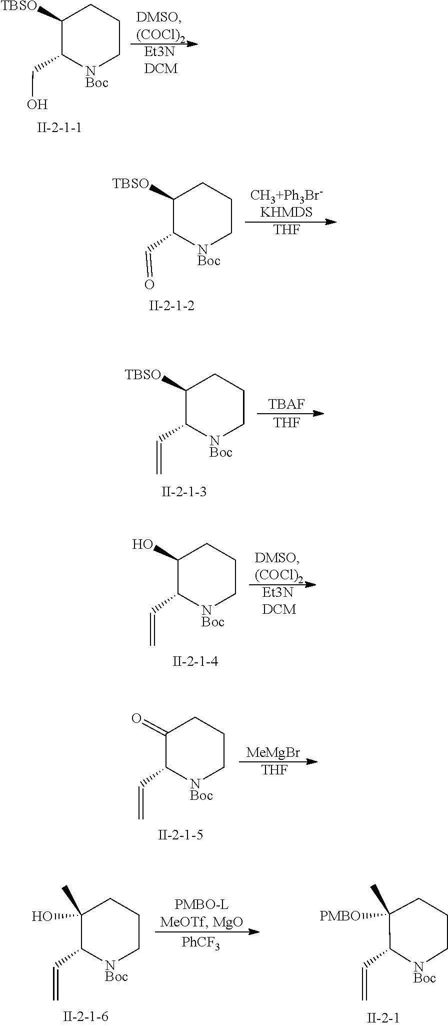

Step 1. A solution of oxalyl chloride (1.25 eq.) in dichloromethane (0.3 M) is cooled to −78° C. Following the dropwise addition of a solution of dimethylsulfoxide (2.5 eq.) in dichloromethane, the reaction mixture is stirred for 5 min. A solution of II-2-1-1 (1 eq.) in dichloromethane is then added to the reaction mixture. After 5 min of stirring, diisopropylethylamine (5 eq.) is added, and the reaction mixture is warmed to ambient temperature. The reaction mixture is then diluted with diethyl ether and water and the layers are separated. The organic layer is washed with water, brine, dried over MgSO₄, filtered, and concentrated in vacuo. Flash chromatography provides II-2-1-2.

Step 2. Methyltriphenylphosphonium bromide (2.4 eq.) in THE (0.3 M) is cooled to a reduced temperature such as −40° C. To the cooled suspension, 0.5M KHMDS in Toluene (2 eq.) is added dropwise and the mixture is stirred for 20 min at −40° C. Aldehyde II-2-1-2 (1 eq. in toluene) is added to the solution and the mixture is allowed to stir until reaction complete. Saturated NH4Cl is added and product is extracted with ethyl acetate, dried, and concentrated to afford crude product which is purified by flash silica chromatography to provide II-2-1-3.

Step 3. To a solution of II-2-1-3 (1 eq.) in dry THF (0.1 M) at a reduced temperature such as 0° C., is added tetra-n-butylammonium fluoride (TBAF) (1.1 eq.) and the resulting solution stirred and allowed to warm to room temperature till reaction complete. The resulting solution is diluted with dichloromethane and quenched with water. The organic layer is dried with brine and magnesium sulfate, followed by solvent reduction in vacuo. The crude product is purified by flash column chromatography to give II-2-1-4.

Step 4. A solution of oxalyl chloride (1.25 eq.) in dichloromethane (0.3 M) is cooled to −78° C. Following the dropwise addition of a solution of dimethylsulfoxide (2.5 eq.) in dichloromethane, the reaction mixture is stirred for 5 min. A solution of II-2-1-4 (1 eq.) in dichloromethane is then added to the reaction mixture. After 5 min of stirring, diisopropylethylamine (5 eq.) is added, and the reaction mixture is warmed to ambient temperature. The reaction mixture is then diluted with diethyl ether and water and the layers is separated. The organic layer is washed with water, brine, dried over MgSO4, filtered, and concentrated in vacuo. Flash chromatography provides II-2-1-5.

Step 5. To a solution of II-2-1-5 (1 eq) in dry THF (0.2 M) at −78° C. is added methylmagnesium bromide (1.5 eq., 3 M in diethyl ether). The mixture is stirred for 1 hour then slowly warmed to 0° C. Saturated NH4Cl is added, and the mixture is extracted with ethyl acetate, dried, and concentrated to afford crude product which is purified by flash silica chromatography to provide II-2-1-6.

Step 5. To an ice-cold mixture of 2-PMBO-lepidine (2 eq.), MgO (2 eq., vacuum-dried), and II-2-1-6 (1 eq.) in benzotrifluoride (PhCF₃, 0.2 M) is added methyl triflate (2 eq.) dropwise. The ice bath is removed, and the reaction mixture is stirred at room temperature until the reaction is complete. The mixture is then diluted with ethyl acetate, decanted away from the MgO residue, washed with water, dried (MgSO4), filtered, concentrated at reduced pressure, and purified on silica gel to yield II-2-1.

### Preparation of tert-butyl (R)-(1-((tert-butyldimethylsilyl)oxy)-2-methylbut-3-en-2-yl)(methyl)carbamate (II-2-2)

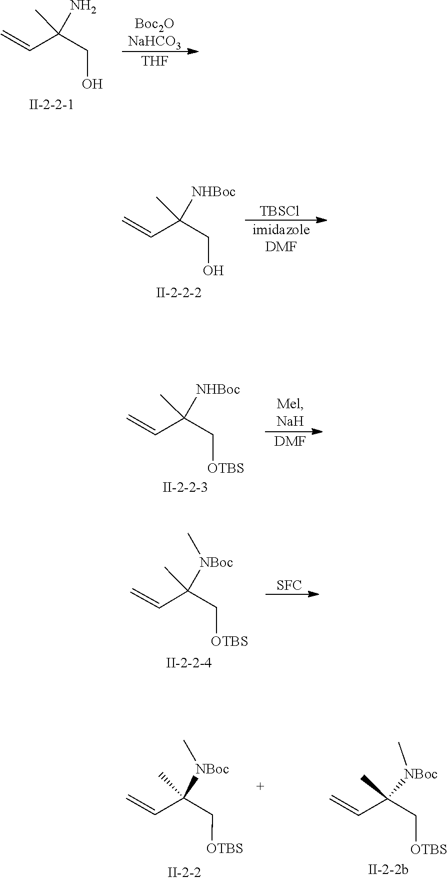

Step 1. To a solution of II-2-2-1 (1 eq.) in THF (0.2 M) is added Boc₂O (1.5 eq.) and saturated bicarbonate solution. The reaction mixture is stirred until complete then diluted with ethyl acetate. The organic layer is removed, washed with brine, dried over MgSO4, filtered, and concentrated in vacuo. Flash chromatography provides II-2-2-2.

Step 2. To a solution of II-2-2-2 (1 eq.) and imidazole (2 eq.) in DMF (0.2 M) at a reduced temperature such as −20° C. is added TBSCl (1.1 eq.). The mixture is warmed to ambient temperature and stirred until reaction complete. The resulting mixture is quenched with water, extracted with EtOAc three times, and the combined extracts are washed with 10% brine 3×, pure brine, and dried over anhydrous Na₂SO₄. The mixture is concentrated under reduced pressure and purified by flash chromatography ethyl acetate in hexanes to afford II-2-2-3.

Step 3. To a cold (0° C.) solution of II-2-2-3 (1 eq.) in dry DMF (0.2 M), is added tetra-sodium hydride (1.6 eq.) and the resulting solution is stirred for 30 minutes at 0° C. To this mixture is added methyl iodide (1.6 eq.) and the reaction is slowly warm to ambient temperature and stirred for 2 hours. Saturated NH₄Cl is added, and the mixture is extracted with EtOAc three times, and the combined extracts are washed with 10% brine three times, pure brine, and dried over anhydrous Na₂SO₄. The mixture is concentrated under reduced pressure and purified by flash chromatography ethyl acetate in hexanes to afford II-2-2-4.

Step 4. Chiral SFC separation afforded desired product II-2-2 and enantiomer II-2-2b.

Intermediate Table 2.

| Comp. # | Structure |
|---|---|
| I-2-4 | 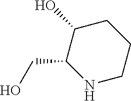 |
| I-2-5 | 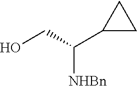 |
| I-2-6 | 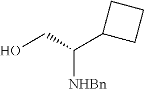 |
| I-2-7 | 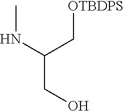 |
| I-2-8 | 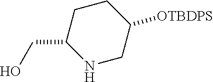 |
| I-2-9 | 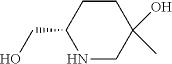 |
| I-2-10 | 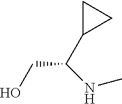 |
| I-2-11 | 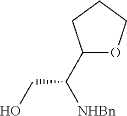 |
| I-2-12 | 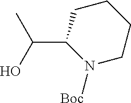 [SMILES: CC(O)[C@@H]1CCCCN1C(=O)OC(C)(C)C] |
| I-2-13 | 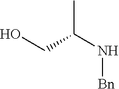 |
| I-2-14 | 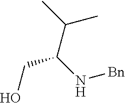 |
| I-2-15 | 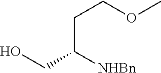 |
| I-2-16 | 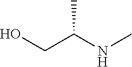 |
| I-2-17 | 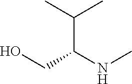 |
| I-2-18 | 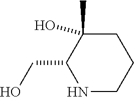 |
| I-2-19 | 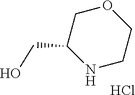 |
| I-2-20 | 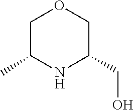 |
| I-2-21 | 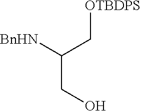 |
| I-2-22 | 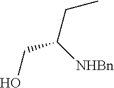 |
| I-2-23 | 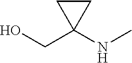 |
| I-2-24 | 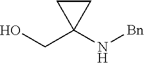 [SMILES: OCC1(NCc2ccccc2)CC1] |
| I-2-25 | 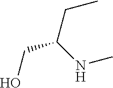 |
| I-2-26 | 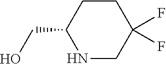 |
| I-2-27 | 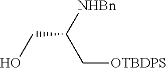 |
| I-2-28 | 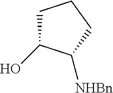 |
| I-2-29 | 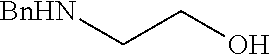 |
| I-2-30 | 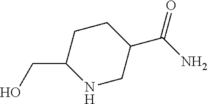 [SMILES: NC(=O)C1CCC(CO)NC1] |
| I-2-31 | 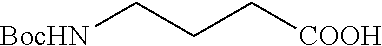 [SMILES: CC(C)(C)OC(=O)NCCCC(=O)O] |
| I-2-32 | 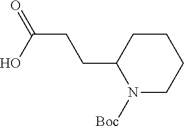 [SMILES: CC(C)(C)OC(=O)N1CCCCC1CCC(=O)O] |
| I-2-33 | 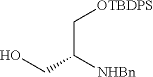 |
| I-2-34 | 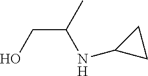 |
| I-2-35 | 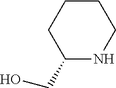 |
| I-2-36 | 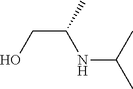 |
| I-2-37 | 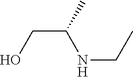 |
| I-2-38 | 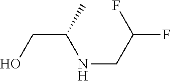 |
| I-2-39 | 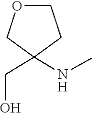 |
| I-2-40 | 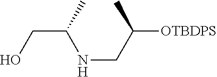 |
| I-2-41 | 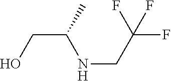 |

### Preparation of (2R,3R)-2-(hydroxymethyl)piperidin-3-ol (I-2-4)

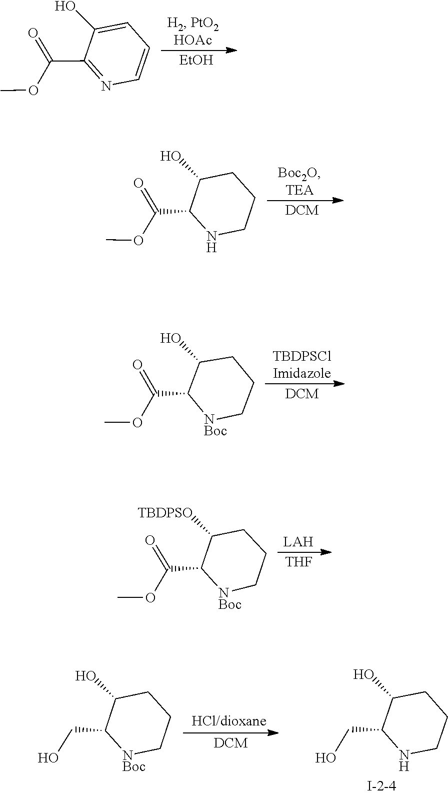

Step 1. To a solution of methyl 3-hydroxypicolinate (10.0 g, 65.3 mmol, 1 eq) in EtOH (100 mL) and HOAc (50 mL) was added PtO₂ (7.41 g, 32.6 mmol, 0.5 eq) under N₂ atmosphere. The suspension was degassed and purged thrice with H₂. The mixture was stirred under H₂ (50 psi) at 40° C. for 72 h. On completion, the reaction mixture was filtered and concentrated under reduced pressure to give methyl (2S,3R)-3-hydroxypiperidine-2-carboxylate (5.00 g, 31.4 mmol, 48% yield) as black oil.

Step 2. To a solution of methyl (2S,3R)-3-hydroxypiperidine-2-carboxylate (3.00 g, 18.8 mmol, 1 eq) in DCM (90 mL) was added Triethylamine (5.72 g, 56.5 mmol, 3 eq) and Boc₂O (6.17 g, 28.3 mmol, 1.5 eq). The mixture was stirred at 25° C. for 16 h. On completion, the reaction mixture was diluted with H₂O (100 mL) and extracted with DCM (50 mL*3). The combined organic layers were washed with brine (100 mL), dried over Na₂SO₄, filtered, and concentrated under reduced pressure to give 1-(tert-butyl) 2-methyl (2S,3R)-3-hydroxypiperidine-1,2-dicarboxylate (2.00 g, 7.71 mmol, 40% yield) as a black solid.

Step 3. To a solution of 01-tert-butyl 02-methyl (2S,3R)-3-hydroxypiperidine-1,2-dicarboxylate (1.50 g, 5.78 mmol, 1 eq) in DCM (15 mL) was added imidazole (787 mg, 11.6 mmol, 2 eq) and TBDPSCl (1.75 g, 6.36 mmol, 1.1 eq). The mixture was stirred at 40° C. for 16 h. On completion, the mixture was concentrated in vacuum to give a crude residue, which was purified by combi flash (40 g silica gel column, EtOAc in PE from 0% to 20%) to give 01-tert-butyl 02-methyl (2S,3R)-3-[tert-butyl(diphenyl)silyl]oxypiperidine-1,2-dicarboxylate (2.55 g, 3.79 mmol, 65% yield, 74% purity) as colorless oil. LCMS: m/z 398.1 (M-100+1).

Step 4: To a solution of 01-tert-butyl 02-methyl (2S,3R)-3-[tert-butyl(diphenyl)silyl]oxypiperidine-1,2-dicarboxylate (2.30 g, 4.62 mmol, 1 eq) in THF (30 mL) was added LiAlH₄ (2 M, 4.62 mL, 2 eq) at 0° C., and the mixture was stirred at 0° C. for 1 h. On completion, the mixture was quenched by sat. NH4Cl (40 mL) and extracted with EtOAc (20 mL*3). The organic layer was dried over anhydrous Na₂SO₄, filtered and the filtrate was concentrated in vacuum to give a crude residue, which was purified by combi flash (20 g silica gel, EtOAc in PE from 0% to 100%) to give tert-butyl (2R,3R)-3-hydroxy-2-(hydroxymethyl)piperidine-t-carboxylate (500 mg, 2.16 mmol, 46% yield) as a white solid. ¹H NMR (400 MHz, MeOD-d₄) δ=4.50-4.37 (m, 1H), 3.97-3.88 (m, 1H), 3.87-3.80 (m, 2H), 3.76-3.67 (m, 1H), 2.92-2.77 (m, 1H), 1.82-1.65 (m, 2H), 1.57-1.48 (m, 2H), 1.48-1.44 (m, 9H).

Step 5: To a solution of tert-butyl (2R,3R)-3-hydroxy-2-(hydroxymethyl)piperidine-1-carboxylate (480 mg, 2.08 mmol, 1 eq) in DCM (5 mL) was added HCl/dioxane (4 M, 5 mL, 9.64 eq) at 0° C., then the mixture was stirred at 25° C. for 1 h. On completion, the mixture was concentrated in vacuum to give (2R,3R)-2-(hydroxymethyl) piperidin-3-ol (326 mg, 1.94 mmol, 94% yield, HCl) as a white solid (intermediate I-2-4).

### Preparation of (S)-2-(benzylamino)-2-cyclopropylethan-1-ol (I-2-5)

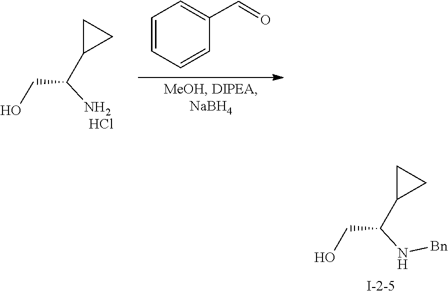

To a solution of (2S)-2-amino-2-cyclopropyl-ethanol (1.00 g, 7.27 mmol, 1 eq, HCl) in MeOH (10 mL) was added benzaldehyde (771 mg, 7.27 mmol, 1 eq) and DIPEA (939 mg, 7.27 mmol, 1 eq). The mixture was stirred at 25° C. for 2 h. NaBH₄ (779 mg, 20.6 mmol, 3 eq) was then added to the reaction mixture. The mixture was stirred at 25° C. for 1 h. On completion, the mixture was quenched by dropwise addition of methanol and concentrated in vacuum to give a residue. The residue was purified by column chromatography (SiO₂, DCM/MeOH=15:1 to 8:1) to give (2S)-2-(benzylamino)-2-cyclopropyl-ethanol (1.10 g, 5.75 mmol, 83% yield) as a colorless oil (intermediate I-2-5). ¹H NMR (400 MHz, DMSO-d₆) δ=7.49-7.16 (m, 5H), 4.71-4.36 (m, 1H), 4.00-3.72 (m, 2H), 3.60-3.44 (m, 2H), 1.82 (dt, J=4.0, 8.0 Hz, 1H), 0.70-0.05 (m, 5H); LCMS: (M+1: 192.1).

### Preparation of (S)-2-(benzylamino)-2-cyclobutylethan-1-ol (I-2-6)

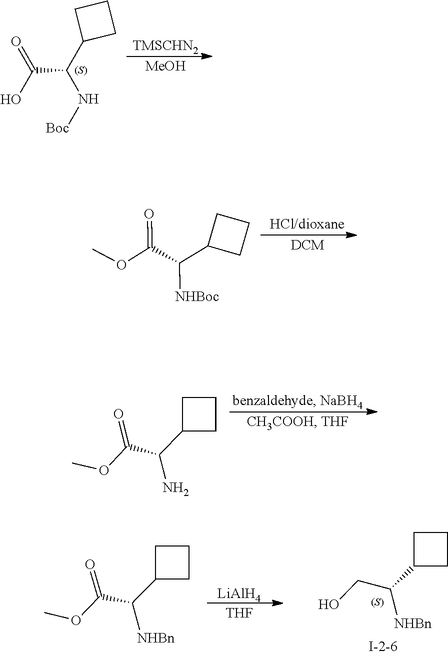

Step 1. A mixture of (2S)-2-(tert-butoxycarbonylamino)-2-cyclobutyl-acetic acid (4.70 g, 20.5 mmol, 1 eq) in MeOH (45 mL) was degassed and purged with N₂ 3 times, and then TMSCHN₂ (2 M, 41 mL, 4 eq) was added at 0° C., the mixture was stirred at 25° C. for 16 h under N₂ atmosphere. On completion, the reaction mixture was concentrated under reduced pressure to give a residue. The residue was purified by column chromatography (SiO₂, Petroleum ether/Ethyl acetate=1:0 to 0:1) to give methyl (2S)-2-(tert-butoxycarbonylamino)-2-cyclobutyl-acetate (3.80 g, 15.6 mmol, 76% yield) as a white solid. ¹H NMR (400 MHz, CDCl₃) δ=5.02-4.92 (m, 1H), 4.24 (t, J=8.0 Hz, 1H), 3.72 (s, 3H), 2.66-2.54 (m, 1H), 2.01-1.79 (m, 7H), 1.45 (s, 9H).

Step 2. To a solution of methyl (2S)-2-(tert-butoxycarbonylamino)-2-cyclobutyl-acetate (2.90 g, 11.9 mmol, 1 eq) in DCM (20 mL) was added HCl/dioxane (4 M, 10.0 mL, 3.4 eq). The mixture was stirred at 25° C. for 0.5 h. On completion, the reaction mixture was concentrated to give methyl (2S)-2-amino-2-cyclobutyl-acetate (1.70 g, 9.46 mmol, 79% yield, HCl) as a white solid.

Step 3. To a solution of methyl (2S)-2-amino-2-cyclobutyl-acetate (1.70 g, 11.9 mmol, 1 eq) and benzaldehyde (1.26 g, 11.9 mmol, 1 eq) in THF (20 mL) was added NaBH₄ (898 mg, 23.8 mmol, 2 eq) and CH₃COOH (119 mmol, 6.80 mL, 10 eq). The mixture was stirred at 25° C. for 0.5 h. On completion, the reaction mixture was partitioned between ethyl acetate (30 mL*3) and water (30 mL), the combined organic phase was dried over anhydrous sodium sulfate, filtered, and concentrated to give a residue. The residue was purified by column chromatography (SiO₂, Dichloromethane:Methanol=1:0 to 20:1) to give methyl (2S)-2-(benzylamino)-2-cyclobutyl-acetate (390 mg, 1.67 mmol, 14% yield) as colorless oil. ¹H NMR (400 MHz, CDCl₃) δ=7.26-7.23 (m, 2H), 7.23-7.22 (m, 1H), 7.20-7.14 (m, 2H), 3.76 (d, J=13.2 Hz, 1H), 3.63 (s, 3H), 3.55 (d, J=13.2 Hz, 1H), 3.12 (d, J=8.4 Hz, 1H), 2.50-2.37 (m, 1H), 2.05-1.91 (m, 1H), 1.90-1.78 (m, 3H), 1.74-1.63 (m, 3H), 1.38 (s, 1H). LCMS: (M+1: 234.1).

Step 4. To a solution of methyl (2S)-2-(benzylamino)-2-cyclobutyl-acetate (340 mg, 1.46 mmol, 1 eq) in THF (3 mL) was added LiAlH₄ (2.5 M, 0.7 mL, 1.2 eq) at 0° C. The mixture was stirred at 25° C. for 5 mines. On completion, the mixture was quenched with water (3 mL), followed by 15% NaOH (3 mL), extracted with ethyl acetate (10 mL*3), and the combined organic phase was dried over anhydrous sodium sulfate, filtered, and concentrated to give (2S)-2-(benzylamino)-2-cyclobutyl-ethanol (290 mg, 1.41 mmol, 97% yield, intermediate I-2-6) as a colorless oil. LCMS: (M+1: 206.1).

### Preparation of 3-((tert-butyldiphenylsilyl)oxy)-2-(methylamino)propan-1-ol (I-2-7)

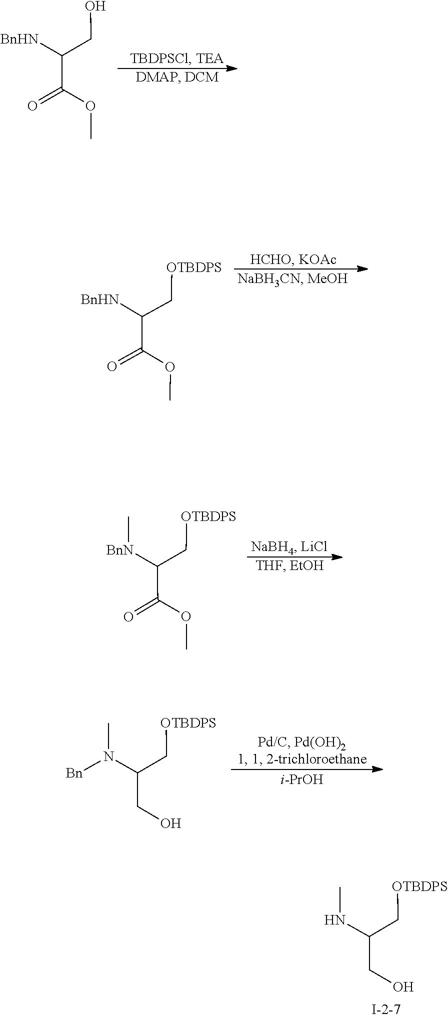

Step 1. To a solution of methyl 2-(benzylamino)-3-hydroxy-propanoate (20.0 g, 95.5 mmol, 1 eq) in DCM (200 mL) was added Triethylamine (11.6 g, 115 mmol, 1.2 eq) and DMAP (583 mg, 4.78 mmol, 0.05 eq), and TBDPSCl (27.6 g, 100 mmol, 1.05 eq) was added at 0° C. The mixture was stirred at 25° C. for 1 h. On completion, the mixture was quenched with dilute citric acid (300 mL) and extracted with DCM (200 mL×3), the combined organic phase was dried over anhydrous Na₂SO₄, filtered and the filtrate was concentrated to give a residue. The residue was purified by column chromatography (SiO₂, PE:THF=5:1) to give methyl 2-(benzylamino)-3-[tert-butyl(diphenyl)silyl]oxy-propanoate (38.5 g, crude) as a white solid. LCMS: (M+1: 448.0).

Step 2. To a solution of methyl 2-(benzylamino)-3-[tert-butyl(diphenyl)silyl]oxy-propanoate (18.5 g, 41.5 mmol, 1 eq), formaldehyde (12.4 g, 415 mmol, 10 eq) in MeOH (200 mL) was added AcOH (41.5 mmol, 2.38 mL, 1 eq), and then the mixture was stirred at 25° C. for 30 min, then NaBH₃CN (7.83 g, 124 mmol, 3 eq) was added. The mixture was stirred at 25° C. for 1 h. On completion, the mixture was diluted with water (4 mL) and concentrated to give a residue. The residue was purified by column chromatography (SiO₂, PE:THF=5:1) to give methyl 2-[benzyl(methyl)amino]-3-[tert-butyl(diphenyl) silyl]oxy-propanoate (15.2 g, crude) as a white oil. LCMS: (M+1: 462.1).

Step 3. To a solution of methyl 2-[benzyl(methyl)amino]-3-[tert-butyl(diphenyl)silyl]oxy-propanoate (10.0 g, 21.6 mmol, 1 eq) in THE (90 mL) and EtOH (10 mL), was added LiCl (5.97 g, 140 mmol, 6.5 eq) and NaBH₄ (8.19 g, 216 mmol, 10 eq) at 0° C. The mixture was stirred at 25° C. for 16 h. On completion, the mixture was diluted with MeOH (150 mL) and concentrated to give a residue. The residue was purified by column chromatography (SiO₂, PE:THF=3:1) to give 2-[benzyl(methyl)amino]-3-[tert-butyl(diphenyl)silyl]oxy-propan-1-ol (9.00 g, crude) as a white oil. LCMS: (M+1: 434.4).

Step 4. To a solution of 2-[benzyl(methyl)amino]-3-[tert-butyl(diphenyl)silyl]oxy-propan-1-ol (9.00 g, 20.7 mmol, 1 eq), 1,1,2-trichloroethane (3.05 g, 22.8 mmol, 1.1 eq) in i-PrOH (90 mL) was added Pd/C (2.25 g, 21.1 mmol, 10% purity, 1.02 eq) and Pd(OH)₂ (11.2 g, 16.0 mmol, 20% purity, 0.772 eq). The mixture was stirred at 25° C. for 16 h. On completion, the mixture was filtered and concentrated to give a residue to give 3-[tert-butyl(diphenyl)silyl]oxy-2-(methylamino)propan-1-ol (6.24 g, crude, HCl) as a white solid. LCMS: (M+1: 344.2).

### Preparation of ((2S,5S)-5-((tert-butyldiphenylsilyl)oxy)piperidin-2-yl)methanol (I-2-8)

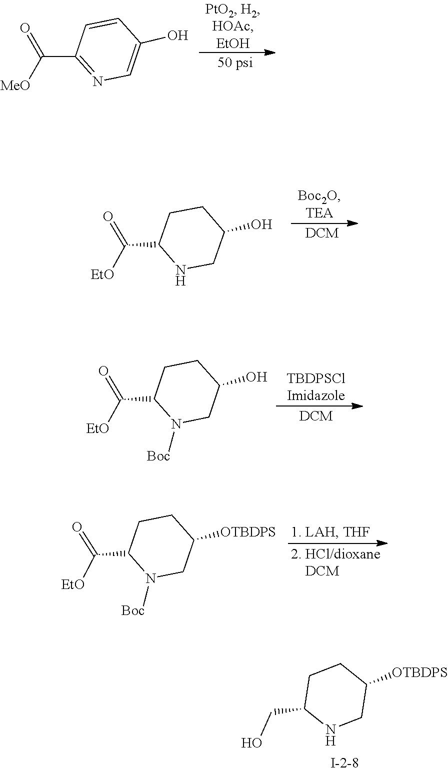

Intermediate I-2-8 was prepared following the same procedure as the preparation of I-2-4.

### Preparation of (6S)-6-(hydroxymethyl)-3-methylpiperidin-3-ol (I-2-9)

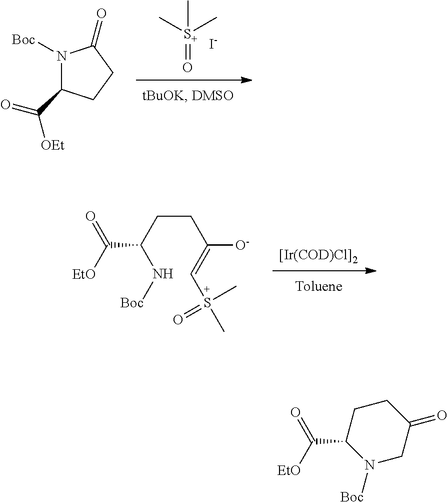

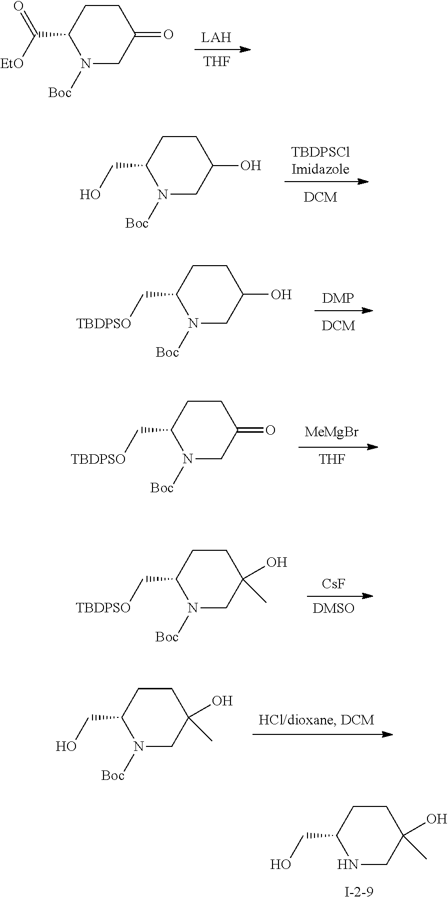

Step 1. To a solution of 01-tert-butyl 02-ethyl (2S)-5-oxopyrrolidine-1,2-dicarboxylate (23.0 g, 89.4 mmol, 1 eq) in DMSO (230 mL) was added t-BuOK (8.03 g, 71.5 mmol, 0.8 eq) and trimethylsulfoxonium iodide (19.6 g, 89.4 mmol, 1 eq). The mixture was stirred at 25° C. for 4 h. On completion, the reaction mixture was diluted with H₂O (600 mL) and extracted with EtOAc (90 mL*20). The combined organic layers were washed with brine (50 mL*2), dried over anhydrous sodium sulfate, filtered, and concentrated to give a residue. The residue was purified by column chromatography (SiO₂, Petroleum ether/Ethyl acetate=1/1 to 0/1, DCM:MeOH=10:1) to give ethyl (S)-2-((tert-butoxycarbonyl)amino)-6-(dimethyl(oxo)-λ⁶-sulfaneylidene)-5-oxohexanoate (30.0 g, 79.8 mmol, 90% yield, 93% purity) as yellow oil. ¹H NMR (400 MHz, DMSO-d₆) δ=7.29 (d, J=7.6 Hz, 1H), 4.75 (s, 1H), 4.16-4.06 (m, 2H), 3.94-3.87 (m, 1H), 3.44 (s, 6H), 2.56 (s, 2H), 1.94-1.82 (m, 1H), 1.79-1.68 (m, 1H), 1.39 (s, 9H), 1.19 (t, J=7.2 Hz, 3H).

Step 2. A mixture of ethyl (S)-2-((tert-butoxycarbonyl)amino)-6-(dimethyl(oxo)-λ6-sulfaneylidene)-5-oxohexanoate (10.0 g, 28.6 mmol, 1 eq) and [Ir(COD)Cl]₂ (1.54 g, 2.29 mmol, 0.08 eq) in Toluene (120 mL) was degassed and purged with N₂ 3 times, and then the mixture was stirred at 80° C. for 16 h under N₂ atmosphere. On completion, the reaction mixture was filtered and concentrated under reduced pressure to give a residue. The residue was purified by column chromatography (SiO₂, Petroleum ether/Ethyl acetate=100/1 to 50/1) to give O1-tert-butyl O2-ethyl (2S)-5-oxopiperidine-1,2-dicarboxylate (4.00 g, 14.7 mmol, 52% yield) as a white solid. ¹H NMR (400 MHz, DMSO-d₆) δ=4.59-4.42 (m, 1H), 4.22-4.12 (m, 2H), 4.11-4.05 (m, 1H), 3.89-3.76 (m, 1H), 2.48-2.40 (m, 1H), 2.34-2.23 (m, 2H), 2.10-1.95 (m, 1H), 1.41-1.33 (m, 9H), 1.25-1.16 (m, 3H).

Step 3. A mixture of 01-tert-butyl 02-ethyl (2R)-5-oxopiperidine-1,2-dicarboxylate (5.00 g, 18.4 mmol, 1 eq), LiAlH₄ (2.5 M, 14.7 mL, 2 eq) in THE (50 mL) was degassed and purged with N₂ 3 times, and then the mixture was stirred at 0° C. for 1 hour under N₂ atmosphere. On completion, the reaction mixture was quenched by addition H₂O (1.5 mL), 15% NaOH (1.5 mL) at 0° C., and then filtered and concentrated under reduced pressure to give a residue. The residue was diluted with H₂O (5 mL) and extracted with EtOAc (50 mL×3). The combined organic layers were washed with NaCl (aq.) (5 mL), dried over anhydrous Na₂SO₄, filtered and concentrated under reduced pressure to give a residue without purification to give tert-butyl (2R)-5-hydroxy-2-(hydroxymethyl)piperidine-1-carboxylate (3.30 g, 14.3 mmol, 77% yield) as colorless oil. ¹H NMR (400 MHz, Chloroform-d) δ=4.32-4.09 (m, 2H), 3.85-3.72 (m, 1H), 3.71-3.54 (m, 2H), 2.65 (t, J=12.0 Hz, 1H), 2.05 (s, 1H), 1.95-1.83 (m, 1H), 1.82-1.75 (m, 1H), 1.72-1.60 (m, 1H), 1.46 (s, 9H).

Step 4. To a solution of tert-butyl (2R,5R)-5-hydroxy-2-(hydroxymethyl)piperidine-1-carboxylate (3.30 g, 14.3 mmol, 1 eq) in DCM (30 mL) was added TBDPSCl (3.14 g, 11.4 mmol, 2.92 mL, 0.8 eq) and Imidazole (1.17 g, 17.1 mmol, 1.2 eq). The mixture was stirred at 25° C. for 2 hours. On completion, the reaction mixture was diluted with H₂O (10 mL) and extracted with DCM (3×10 mL). The combined organic layers were washed with brine (5 mL), dried over Na₂SO₄, filtered, and concentrated under reduced pressure to give a residue. The residue was purified by column chromatography (SiO₂, Petroleum ether/Ethyl acetate=100/1 to 0/1) to give tert-butyl (2R)-2-(((tert-butyldiphenylsilyl)oxy)methyl)-5-hydroxypiperidine-1-carboxylate (5.70 g, 10.9 mmol, 76% yield, 90% purity) as white oil. LCMS: (M+1: 470.1).

Step 5. To a solution of tert-butyl (2R)-2-(((tert-butyldiphenylsilyl)oxy)methyl)-5-hydroxypiperidine-1-carboxylate (5.70 g, 12.1 mmol, 1 eq) in DCM (60 mL) was added DMP (7.72 g, 18.2 mmol, 1.5 eq). The mixture was stirred at 25° C. for 1 hour. On completion, the reaction mixture was quenched by addition Na₂S₂O₃ (2 mL) at 25° C., and then diluted with H₂O (10 mL) and extracted with DCM (3×10 mL). The combined organic layers were washed with brine (5 mL), dried over anhydrous Na₂SO₄, filtered and concentrated under reduced pressure to give a residue, which was purified by column chromatography (SiO₂, Rf=0.6 Petroleum ether/Ethyl acetate=100/1 to 0/1) to give the tert-butyl (R)-2-(((tert-butyldiphenylsilyl)oxy)methyl)-5-oxopiperidine-1-carboxylate (3.40 g, 6.54 mmol, 54% yield, 90% purity) as colorless oil. ¹H NMR (400 MHz, Chloroform-d) δ=7.67-7.60 (m, 4H), 7.48-7.36 (m, 6H), 4.66-4.12 (m, 2H), 4.05-3.72 (m. 2H), 3.71-3.65 (m, 1H), 2.54-2.37 (m, 2H), 2.24-2.07 (m, 2H), 1.49-1.36 (m, 9H), 1.05 (s, 9H); LCMS: (M+23: 490.1).

Step 6. A mixture of tert-butyl (2R)-2-[[tert-butyl(diphenyl)silyl]oxymethyl]-5-oxo-piperidine-1-carboxylate (3.40 g, 7.27 mmol, 1 eq) in THE (30 mL) was degassed and purged with N₂ 3 times, and then the added MeMgBr (2.5 M, 5.82 mL, 2 eq) at −78° C. The mixture was stirred at 25° C. for 2 hours under N₂ atmosphere. On completion, the reaction mixture was quenched by addition NH₄Cl (1 mL) at 0° C., and then diluted with H₂O (5 mL) and extracted with EtOAc (3×5 mL). The combined organic layers were washed with brine (2 mL). dried over anhydrous Na₂SO₄, filtered and concentrated under reduced pressure to give a residue without purification to give the tert-butyl (2R)-2-[[tert-butyl(diphenyl)silyl]oxymethyl]-5-hydroxy-5-methyl-piperidine-1-carboxylate (1.80 g, 3.35 mmol, 46% yield, 90% purity) as white oil. ¹H NMR (400 MHz, CDCl₃-d) δ=7.68-7.65 (m, 4H), 7.42-7.38 (m, 6H), 4.39-4.32 (m, 1H), 3.80 (d, J=12.8 Hz, 1H), 3.73-3.63 (m, 3H), 2.60 (d, J=13.2 Hz, 1H), 1.89-1.82 (m, 1H), 1.74-1.64 (m, 2H), 1.60-1.57 (m, 1H), 1.44 (s, 9H), 1.26-1.23 (m, 3H), 1.05 (s, 9H); LCMS: (M+1: 484.1).

Step 7. To a solution of tert-butyl (2S)-2-[[tert-butyl(diphenyl)silyl]oxymethyl]-5-hydroxy-5-methyl-piperidine-1-carboxylate (1.00 g, 2.07 mmol, 1 eq) in DMSO (2.5 mL) was added CsF (3.14 g, 20.7 mmol, 10 eq). The mixture was stirred at 25° C. for 16 hours. On completion, the reaction mixture was filtered and concentrated under reduced pressure to give a residue. Which was purified by column chromatography (SiO₂, Petroleum ether/Ethyl acetate=100/1 to 0/1). to give the tert-butyl (2S)-5-hydroxy-2-(hydroxymethyl)-5-methyl-piperidine-1-carboxylate (460 mg, 1.84 mmol, 89% yield, 98% purity) as yellow oil. ¹H NMR (400 MHz, DMSO-d₆) δ=4.74-4.46 (m, 1H), 4.01 (s, 2H), 3.58 (d, J=12.0 Hz, 2H), 3.46-3.39 (m, 4H), 3.34 (d, J=6.0 Hz, 2H), 1.70-1.64 (m, 1H), 1.55-1.48 (m, 2H), 1.38 (s, 9H); LCMS: (M-100: 145.8).

Step 8. To a solution of tert-butyl (2S)-5-hydroxy-2-(hydroxymethyl)-5-methyl-piperidine-1-carboxylate (460 mg, 1.88 mmol, 1 eq) in DCM (5 mL) was added HCl/dioxane (4 M, 2.5 mL, 5.33 eq). The mixture was stirred at 25° C. for 1 hour. On completion, the reaction mixture was concentrated under reduced pressure to give a residue to give the (6S)-6-(hydroxymethyl)-3-methyl-piperidin-3-ol (250 mg, 1.55 mmol, 83% yield, 90% purity) as yellow oil (I-2-9). LCMS: (M+1: 145.8).

### Preparation of (S)-2-cyclopropyl-2-(methylamino)ethan-1-ol (I-2-10)

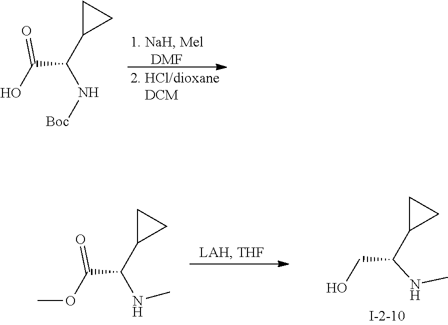

Step 1. To a solution of (2S)-2-(tert-butoxycarbonylamino)-2-cyclopropyl-acetic acid (4.00 g, 18.6 mmol, 1 eq) in DMF (40 mL) was added NaH (2.23 g, 55.8 mmol, 60% purity, 3 eq) at 0° C. for 30 min. Then Mel (7.91 g, 55.8 mmol, 3.47 mL, 3 eq) was added to the mixture and stirred at 25° C. for 5 hr. On completion, the reaction mixture was quenched by water (100 mL) and extracted with EtOAc (35 mL*3). The combined organic layer was dried over anhydrous Na₂SO₄, filtered and the filtrate was concentrated under reduced pressure to give methyl (2S)-2-[tert-butoxycarbonyl(methyl)amino]-2-cyclopropyl-acetate (4.50 g, crude) as colorless oil. LCMS: (M-100=144.1).

To a solution of the above methyl (2S)-2-[tert-butoxycarbonyl(methyl)amino]-2-cyclopropyl-acetate (4.50 g, 18.5 mmol, 1 eq) in DCM (20 mL) was added HCl/dioxane (4.00 M, 21.2 mL, 4.59 eq). The mixture was stirred at 25° C. for 1 hr. On completion, the reaction mixture was concentrated under reduced pressure to give methyl (2S)-2-cyclopropyl-2-(methylamino) acetate (2.50 g, 17.5 mmol, 94% yield) as white solid.

Step 2. To a solution of methyl (2S)-2-cyclopropyl-2-(methylamino) acetate (2.50 g, 17.5 mmol, 1 eq) in THF (30 mL) was added LAH (994 mg, 26.2 mmol, 1.5 eq). The mixture was stirred at 0° C. for 2 hr. On completion, the reaction mixture was quenched by water (2 mL) and concentrated under reduced pressure to give a residue. The residue was purified by combi flash (20.0 g silica gel column, MeOH in DCM from 0% to 100%) to give (2S)-2-cyclopropyl-2-(methylamino) ethanol (1.90 g, 16.5 mmol, 94% yield) as colorless oil (Intermediate I-2-10).

### Preparation of (2R)-2-(benzylamino)-2-(tetrahydrofuran-2-yl)ethan-1-ol (I-2-11)

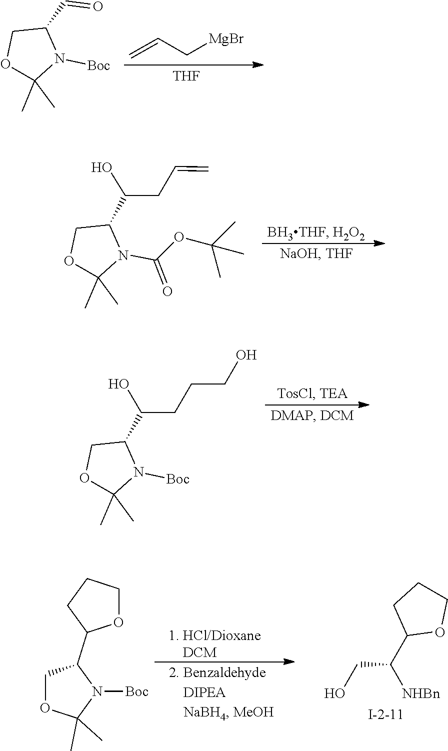

Step 1. A mixture of tert-butyl (4R)-4-formyl-2,2-dimethyl-oxazolidine-3-carboxylate (4.93 g, 21.5 mmol, 1 eq) in THE (50 mL) was degassed and purged with N₂ 3 times, and then allyl(bromo)magnesium (1 M, 28 mL, 1.3 eq) was added at 0° C., the mixture was stirred at 0° C. for 1 h under N₂ atmosphere. On completion, the mixture was quenched with NH₄Cl (50 mL) and extracted with ethyl acetate (25 mL×3), the combined organic phase was dried over anhydrous sodium sulfate, filtered, and concentrated to give a residue. The residue was purified by column chromatography (SiO₂, Petroleum ether/Ethyl acetate=1:0 to 3:1) to give tert-butyl (4R)-4-(1-hydroxybut-3-enyl)-2, 2-dimethyl-oxazolidine-3-carboxylate (5.28 g, 19.5 mmol, 90% yield) as a yellow oil. ¹H NMR (400 MHz, DMSO-d₆) δ=5.93-5.74 (m, 1H), 5.10-4.93 (m, 2H), 4.91-4.74 (m, 1H), 4.09-3.93 (m, 1H), 3.92-3.55 (m, 3H), 2.18-1.93 (m, 2H), 1.52-1.34 (m, 15H).

Step 2. To a solution of tert-butyl (4R)-4-[(1S)-1-hydroxybut-3-enyl]-2,2-dimethyl-oxazolidine-3-carboxylate (4.76 g, 17.5 mmol, 1 eq) in THF (55 mL) was added BH₃·THF (1 M, 70 mL, 4 eq) dropwise at 0° C. After stirring at 0° C. for 1 h, NaOH (2 M, 70 mL, 8 eq) was added dropwise and stirred at 0° C. Then H₂O₂ (5.97 g, 52.6 mmol, 30% purity, 3 eq) was added dropwise and the mixture was stirred for 1 h at 0° C. On completion, the mixture was quenched with sat. Na₂SO₃ (130 ml) and extracted with 2-Me THE (100 mL×3), the combined organic phase was dried over anhydrous sodium sulfate, filtered, and concentrated to give a residue. The residue was purified by column chromatography (SiO₂, PE/EtOAc=1:0 to 1:3) to give tert-butyl (4R)-4-(1,4-dihydroxybutyl)-2,2-dimethyl-oxazolidine-3-carboxylate (4.25 g, 14.7 mmol, 84% yield) as a yellow oil. ¹H NMR (400 MHz, DMSO-d6) δ=4.79-4.65 (m, 1H), 4.36 (br d, J=2.9 Hz, 1H), 3.98-3.89 (m, 1H), 3.88-3.72 (m, 2H), 3.43-3.34 (m, 2H), 1.48 (s, 1H), 1.50-1.46 (m, 1H), 1.46-1.31 (m, 15H).

Step 3. To a solution of tert-butyl (4R)-4-(1,4-dihydroxybutyl)-2,2-dimethyl-oxazolidine-3-carboxylate (4.25 g, 14.7 mmol, 1 eq) in DCM (150 mL) was added Triethylamine (4.46 g, 44.1 mmol, 3 eq), DMAP (449 mg, 3.67 mmol, 0.25 eq) and Tosyl Chloride (3.08 g, 16.2 mmol, 1.1 eq). The mixture was stirred at 25° C. for 48 h. On completion, the mixture was quenched with water (150 mL) and extracted with DCM (50 mL×3), the combined organic phase was dried over anhydrous sodium sulfate, filtered, and concentrated to give a residue. The residue was purified by column chromatography (SiO₂, Petroleum ether/Ethyl acetate=1:0 to 10:1) to give tert-butyl (4R)-2,2-dimethyl-4-tetrahydrofuran-2-yl-oxazolidine-3-carboxylate (3.08 g, 11.4 mmol, 77% yield) as a colorless oil. ¹H NMR (400 MHz, CDCl3-d) δ=4.25-4.16 (m, 1H), 4.16-4.09 (m, 1H), 4.09-4.04 (m, 1H), 3.99-3.84 (m, 2H), 3.71 (br d, J=1.9 Hz, 1H), 1.74 (br s, 3H), 1.68-1.55 (m, 3H), 1.54-1.42 (m, 9H), 1.37-1.22 (m, 1H).

Step 4. To a solution of tert-butyl (4R)-2, 2-dimethyl-4-tetrahydrofuran-2-yl-oxazolidine-3-carboxylate (2.52 g, 9.29 mmol, 1 eq) in DCM (24 mL) was added HCl/dioxane (4 M, 23 mL, 10 eq). The mixture was stirred at 25° C. for 0.5 h. On completion, the mixture was concentrated to give (2R)-2-amino-2-tetrahydrofuran-2-yl-ethanol (1.56 g, 9.31 mmol, 100% yield, HCl) as a brown oil.

To a solution of the above (2R)-2-amino-2-tetrahydrofuran-2-yl-ethanol (1.56 g, 11.9 mmol, 1 eq) and benzaldehyde (1.26 g, 11.9 mmol, 1 eq) in MeOH (80 mL) was added DIPEA (3.07 g, 23.8 mmol, 2 eq). The mixture was stirred at 25° C. for 2 h, and then NaBH₄ (2.25 g, 59.5 mmol, 5 eq) was added, the mixture was stirring at 25° C. for 1 h. On completion, the mixture was concentrated to give a residue. The residue was purified by column chromatography (SiO₂, PE/THF=1:0 to 1:1) to give (2R)-2-(benzylamino)-2-tetrahydrofuran-2-yl-ethanol (381 mg, 1.72 mmol, 14% yield) as a colorless oil (I-2-11). LCMS: (M+1: 222.1).

### Preparation of tert-butyl (2S)-2-(1-hydroxyethyl)piperidine-1-carboxylate (I-2-12)

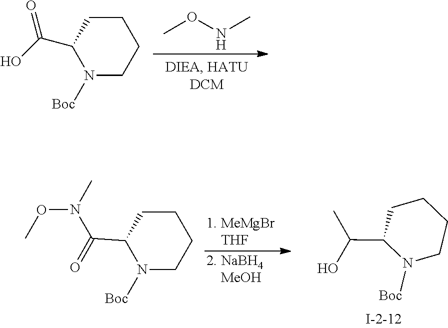

Step 1. A mixture of (2S)-1-tert-butoxycarbonylpiperidine-2-carboxylic acid (5.00 g, 21.8 mmol, 1 eq), N-methoxymethanamine hydrochloride (2.34 g, 24.0 mmol, 1.1 eq), DIPEA (5.64 g, 43.6 mmol, 2 eq), HATU (9.12 g, 24.0 mmol, 1.1 eq) in DCM (50 mL) was stirred at 25° C. for 2 h. On completion, the mixture was poured into water (100 mL), the aqueous phase was extracted with DCM (50 mL×2). The combined organic phase was washed with brine (20 mL×2), dried over anhydrous Na₂SO₄, filtered, and concentrated under vacuum. The residue was purified by column chromatography (SiO₂, Petroleum ether/Ethyl acetate=1/0 to 1/1) to give tert-butyl (2S)-2-[methoxy(methyl)carbamoyl]piperidine-1-carboxylate (4.80 g, 17.6 mmol, 80% yield) as a colorless oil. LCMS: (M+1-100:173.2).

Step 2. To a solution of tert-butyl (2S)-2-[methoxy(methyl)carbamoyl]piperidine-1-carboxylate (2.50 g, 9.18 mmol, 1 eq) in THF (25 mL) was added MeMgBr (3 M, 9.18 mL, 3 eq) at −78° C. The mixture was stirred at 25° C. for 2 h. On completion, the mixture was poured into NH₄Cl (30 mL) aqueous solution, the aqueous phase was extracted with ethyl acetate (10 mL×3). The combined organic phase was washed with brine (10 ml×2), dried over anhydrous Na₂SO₄, filtered and concentrated under vacuum. The residue was purified by column chromatography (SiO₂, Petroleum ether/Ethyl acetate=5/1 to 4/1) to give tert-butyl (2S)-2-acetylpiperidine-1-carboxylate (1.51 g, 6.64 mmol, 72% yield) as a colorless oil. LCMS: (M+1-100:128.2).

To a solution of the above tert-butyl (2S)-2-acetylpiperidine-1-carboxylate (1.40 g, 6.16 mmol, 1 eq) in MeOH (14 mL) was added NaBH₄ (500 mg, 13.2 mmol, 2.15 eq) at 0° C. The mixture was stirred at 25° C. for 2 h. On completion, the mixture was poured into water (30 mL), the aqueous phase was extracted with ethyl acetate (10 mL×3). The combined organic phase was washed with brine (10 mL×2), dried over anhydrous Na₂SO₄, filtered, and concentrated under vacuum. The residue was purified by column chromatography (SiO₂, Petroleum ether/Ethyl acetate=1/0 to 4/1) to give tert-butyl (2S)-2-(1-hydroxyethyl) piperidine-1-carboxylate (1.25 g, 5.45 mmol, 88% yield) as a colorless oil (I-2-12). LCMS: (M+1-100:130.2).

Intermediate I-2-13 is commercially available.

### Preparation of (S)-2-(benzylamino)-3-methylbutan-1-ol (I-2-14)

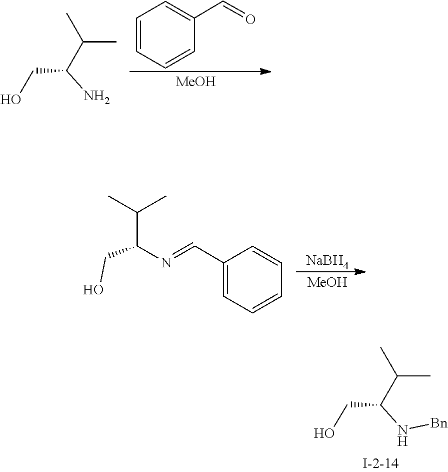

Step 1: To a solution of (2S)-2-amino-3-methyl-butan-1-ol (3.00 g, 29.1 mmol, 1.00 eq) in MeOH (30 ml) was added benzaldehyde (3.09 g, 29.1 mmol, 1.00 eq). The mixture was stirred at 25° C. for 1.5 h. On completion, the mixture was concentrated to give (2S)-2-[(E)-benzylideneamino]-3-methyl-butan-1-ol (5.56 g, crude) as black liquid. LCMS: (M+1=192.0).

Step 2: To a solution of (2S)-2-[(E)-benzylideneamino]-3-methyl-butan-1-ol (5.50 g, 28.8 mmol, 1.00 eq) in MeOH (50.0 mL) was added NaBH₄ (3.26 g, 86.3 mmol, 3.00 eq) in batches at 0° C. The mixture was stirred at 25° C. for 1 hr. On completion, the mixture was quenched with MeOH (10.0 mL) until no gas formed and concentrated in vacuum to give a residue. The residue was purified by column chromatography (SiO₂, DCM/MeOH=1:0 to 10:1) to give (2S)-2-(benzylamino)-3-methyl-butan-1-ol (3.90 g, 20.2 mmol, 70% yield) as colorless oil. ¹H NMR (400 MHz, DMSO-d₆) δ=7.38-7.16 (m, 5H), 4.38 (br s, 1H), 3.77-3.66 (m, 2H), 3.45 (dd, J=4.8, 10.8 Hz, 1H), 3.29 (br s, 1H), 2.27 (q, J=5.2 Hz, 1H), 1.91-1.71 (m, 2H), 0.86 (dd, J=7.2, 8.8 Hz, 6H); LCMS: (M+1=194.2).

### Preparation of (S)-2-(benzylamino)-4-methoxybutan-1-ol (I-2-15)

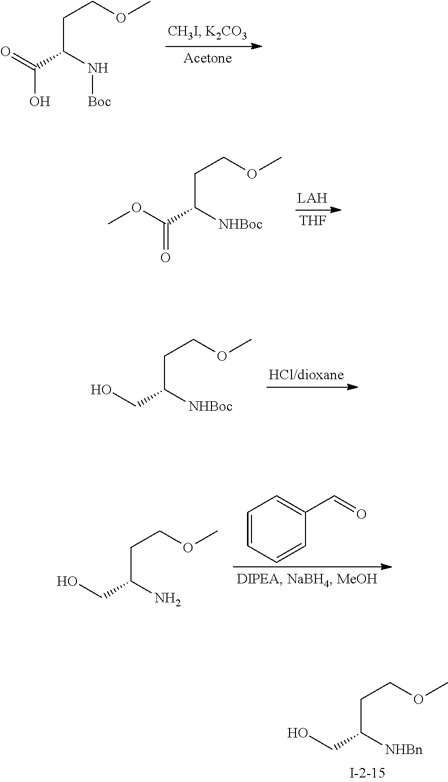

Step 1. To a solution of (2S)-2-(tert-butoxycarbonylamino)-4-methoxy-butanoic acid (4.50 g, 19.3 mmol, 1 eq) in Acetone (45 mL) was added CH₃I (2.74 g, 19.3 mmol, 1.2 mL, 1 eq) and K₂CO₃ (2.67 g, 19.3 mmol, 1 eq). The mixture was stirred at 60° C. for 12 h. On completion, the reaction mixture was partitioned between ethyl acetate (40 mL×3) and water (40 mL), the combined organic phase was dried over anhydrous sodium sulfate, filtered, and concentrated to give a residue. The residue was purified by column chromatography (SiO₂, PE:THF=3:1 to 1:3) to give methyl (2S)-2-(tert-butoxycarbonylamino)-4-methoxy-butanoate (3.94 g, 15.9 mmol, 83% yield) as a brown solid. ¹H NMR (400 MHz, CDCl₃) δ=5.41 (br d, J=6.4 Hz, 1H), 3.74 (s, 3H), 3.53-3.39 (m, 2H), 3.31 (s, 3H), 2.15-1.90 (m, 2H), 1.45 (s, 9H).

Step 2. To a solution of methyl (2S)-2-(tert-butoxycarbonylamino)-4-methoxy-butanoate (3.94 g, 15.9 mmol, 1 eq) in THE (40 mL) was added LiAlH₄ (2.5 M, 6.37 mL, 1 eq) at 0° C. under N₂. The mixture was stirred at 25° C. for 1 h. On completion, the mixture was filtered and concentrated to give a residue. The residue was purified by column chromatography (SiO₂, PE:THF=2:1 to 1:2) to give tert-butyl N-[(1S)-1-(hydroxymethyl)-3-methoxy-propyl]carbamate (2.67 g, 12.2 mmol, 76% yield) as a brown solid. ¹H NMR (400 MHz, CDCl₃) δ=3.76 (br s, 1H), 3.67-3.60 (m, 2H), 3.54-3.42 (m, 2H), 3.39-3.31 (m, 3H), 1.95-1.72 (m, 2H), 1.50-1.39 (m, 9H).

Step 3. To a solution of tert-butyl N-[(1S)-1-(hydroxymethyl)-3-methoxy-propyl]carbamate (2.67 g, 12.2 mmol, 1 eq) in DCM (26 mL) was added HCl/dioxane (4 M, 5 mL, 1.64 eq). The mixture was stirred at 25° C. for 2 h. On completion, the mixture was concentrated to give (2S)-2-amino-4-methoxy-butan-1-ol (2.74 g, crude, HCl) as a white solid.

To a solution of (2S)-2-amino-4-methoxy-butan-1-ol (2.74 g, 23.0 mmol, 1 eq) in MeOH (30 mL) was added benzaldehyde (2.44 g, 23.0 mmol, 1 eq) and DIPEA (5.94 g, 6.0 mmol, 2 eq), the mixture was stirred at 25° C. for 0.5 h, then NaBH₄ (1.30 g, 34.5 mmol, 1.5 eq) was added at 0° C. The mixture was stirred at 25° C. for 12 h. On completion, the reaction mixture was partitioned between EtOAc (30 mL×3) and water (30 mL), the combined organic phase was dried over anhydrous sodium sulfate, filtered, and concentrated to give a residue. The residue was purified by column chromatography (SiO₂, PE:THF=2:1 to 1:2) to give (2S)-2-(benzylamino)-4-methoxy-butan-1-ol (1.36 g, 6.48 mmol, 28% yield) as a brown solid (I-2-15). ¹H NMR (400 MHz, CDCl₃) δ=7.55 (s, 5H), 4.09 (s, 2H), 3.99-3.92 (m, 1H), 3.75 (t, J=6.0 Hz, 2H), 3.72-3.65 (m, 1H), 3.61 (s, 3H), 3.16-3.08 (m, 1H), 2.18-1.94 (m, 2H).

Intermediate I-2-16 is commercially available.

### Preparation of (S)-3-methyl-2-(methylamino)butan-1-ol (I-2-17)

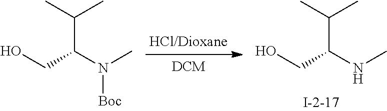

To a solution of tert-butyl N-[(1S)-1-(hydroxymethyl)-2-methyl-propyl]-N-methyl-carbamate (900 mg, 4.14 mmol, 1 eq) in DCM (9 mL) was added HCl/Dioxane (4 M, 1 mL, 1 eq), the mixture was stirred at 25° C. for 1 h. On completion, the mixture was concentrated to give (2S)-3-methyl-2-(methylamino) butan-1-ol (450 mg, 3.84 mmol, 92% yield) as a white solid (I-2-17).

### Preparation of (2R,3R)-2-(hydroxymethyl)-3-methylpiperidin-3-ol (I-2-18)

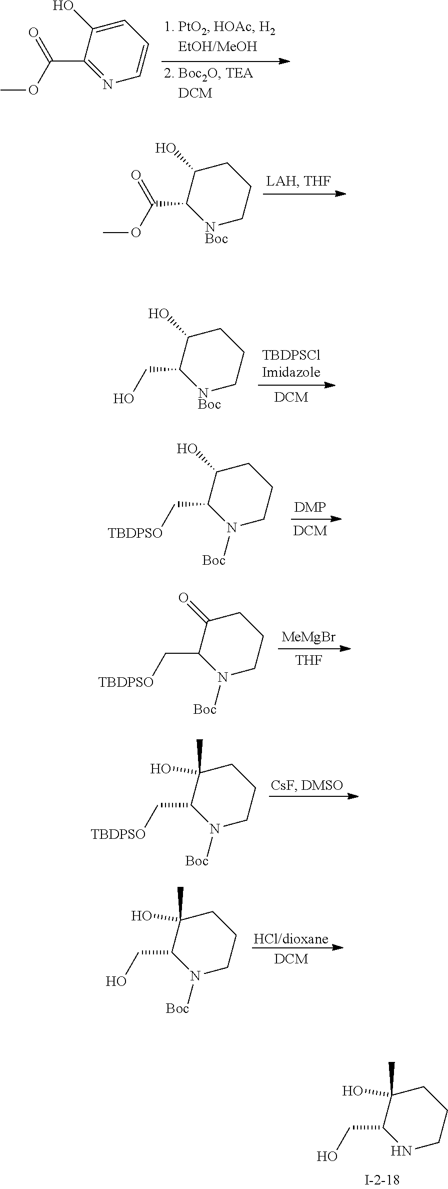

Step 1. PtO₂ (11.1 g, 48.9 mmol, 0.3 eq) was moistened with ethanol (50 mL) under Ar, and then, a solution of methyl 3-hydroxypyridine-2-carboxylate (25.0 g, 163 mmol, 1 eq) in methanol (200 mL) and acetic acid (698 mmol, 40 mL, 4.28 eq) was added. The suspension was degassed under vacuum, purged with H₂ several times, and stirred under H₂ (50 psi) at 40° C. for 16 h. On completion, the reaction mixture was filtered and concentrated under reduced pressure to give methyl (2R,3S)-3-hydroxypiperidine-2-carboxylate (25.9 g, 162 mmol, 99% yield, acetic acid salt) as yellow oil. LCMS: (M+1: 159.8).

To a solution of methyl (2R,3S)-3-hydroxypiperidine-2-carboxylate (25.9 g, 163 mmol, 1 eq) in DCM (260 mL) was added Boc₂O (42.7 g, 195 mmol, 1.2 eq) and Triethylamine (49.5 g, 489 mmol, 3 eq). The mixture was stirred at 25° C. for 2 h. On completion, the reaction mixture was diluted with H₂O (150 mL) and extracted with DCM (50 mL×3). The combined organic layers were washed with brine (30 mL×2), dried over anhydrous sodium sulfate, filtered, and concentrated to give a residue. The residue was purified by column chromatography (SiO₂, Petroleum ether/Ethyl acetate=100/1 to 20/1) to give 01-tert-butyl 02-methyl (2S,3R)-3-hydroxypiperidine-1,2-dicarboxylate (16.0 g, 54.9 mmol, 34% yield, 89% purity) as colorless oil. ¹H NMR (400 MHz, DMSO-d₆) δ=5.29 (d, J=4.0 Hz, 1H), 4.78-4.51 (m, 1H), 3.77-3.66 (m, 2H), 3.61 (s, 3H), 3.24-2.99 (m, 1H), 1.73-1.60 (m, 2H), 1.58-1.46 (m, 1H), 1.45-1.39 (m, 1H), 1.37 (s, 9H); LCMS: (M+100: 160.1).

Step 2. A mixture of 01-tert-butyl 02-methyl (2S,3R)-3-hydroxypiperidine-1,2-dicarboxylate (5.00 g, 19.3 mmol, 1 eq) in THF (60 mL) was degassed and purged with N₂ 3 times at 0° C., LiAlH₄ (2.5 M, 11.5 mL, 1.5 eq) was added, and then the mixture was stirred at 0° C. for 2 hours under N₂ atmosphere. On completion, the reaction mixture was quenched by addition H₂O (1.5 mL), 15% NaOH (1.5 mL) and H₂O (3 mL) at 0° C., and then filtered and concentrated under reduced pressure to give crude product. The crude product was triturated with EtOAc (100 mL) at 25° C. for 10 minutes to give tert-butyl (2R,3R)-3-hydroxy-2-(hydroxymethyl) piperidine-1-carboxylate (2.60 g, 10.2 mmol, 53% yield, 91% purity) as a white solid. ¹H NMR (400 MHz, DMSO-d₆) δ=4.98 (d, J=2.4 Hz, 1H), 4.28 (s, 1H), 4.19 (s, 1H), 3.73 (d, J=12.4 Hz, 1H), 3.63 (d, J=5.6 Hz, 2H), 3.52 (s, 1H), 2.72 (s, 1H), 1.63-1.52 (m, 2H), 1.46-1.40 (m, 1H), 1.38 (s, 9H), 1.33-1.24 (m, 1H); LCMS: (M-56:174.1).

Step 3. To a solution of tert-butyl (2S,3S)-3-hydroxy-2-(hydroxymethyl)piperidine-1-carboxylate (550 mg, 2.38 mmol, 1 eq) in DCM (5 mL) was added TBDPS-Cl (458 mg, 1.66 mmol, 0.7 eq) and imidazole (194 mg, 2.85 mmol, 1.2 eq). The mixture was stirred at 40° C. for 1 hour. On completion, the reaction mixture was diluted with H₂O (15 ml) and extracted with DCM (10 mL×3). The combined organic layers were washed with brine (5 mL×2), dried over Na₂SO₄, filtered and concentrated under reduced pressure to give a residue. The residue was purified by prep-TLC (SiO₂, EtOAc:PE=1:5) to give tert-butyl (2R,3R)-2-[[tert-butyl(diphenyl)silyl]oxymethyl]-3-hydroxy-piperidine-1-carboxylate (550 mg, 1.14 mmol, 48% yield, 97% purity) as yellow oil. ¹H NMR (400 MHz, DMSO-d6) δ=7.63 (t, J=6.4 Hz, 4H), 7.50-7.38 (m, 6H), 5.03 (d, J=3.6 Hz, 1H), 4.56-4.33 (m, 1H), 3.90 (d, J=9.2 Hz, 1H), 3.86-3.81 (m, 1H), 3.54 (d, J=4.0 Hz, 1H), 2.69-2.57 (m, 1H), 1.62-1.49 (m, 2H), 1.39 (s, 9H), 1.31 (d, J=8.8 Hz, 2H), 0.97 (s, 9H); LCMS: (M+1: 470.2).

Step 4. To a solution of tert-butyl (2R,3R)-2-[[tert-butyl(diphenyl)silyl]oxymethyl]-3-hydroxy-piperidine-t-carboxylate (550 mg, 1.17 mmol, 1 eq) in DCM (5 mL) was added DMP (745 mg, 1.76 mmol, 1.5 eq). The mixture was stirred at 25° C. for 2 h. On completion, the reaction mixture was quenched by Na₂S₂O₃ (1 mL) diluted with NaHCO₃ (3 mL) and extracted with DCM (8 mL×3). The combined organic layers were washed with brine (4 mL×2), dried over Na₂SO₄, filtered, and concentrated under reduced pressure to give a residue. The residue was purified by prep-TLC (SiO₂, PE:EtOAc=5:1) to give tert-butyl (2R)-2-[[tert-butyl(diphenyl)silyl]oxymethyl]-3-oxo-piperidine-1-carboxylate (425 mg, 0.899 mmol, 77% yield, 99% purity) as a white solid. ¹H NMR (400 MHz, DMSO-d₆) δ=7.58-7.54 (m, 4H), 7.51-7.40 (m, 6H), 4.41-4.25 (m, 1H), 3.96-3.91 (m, 1H), 3.83-3.78 (m, 1H), 3.38 (d, J=9.6 Hz, 1H), 2.57-2.51 (m, 2H), 2.45-2.36 (m, 1H), 1.99-1.87 (m, 2H), 1.46-1.27 (m, 9H), 0.96 (s, 9H); LCMS: (M+23: 490.1).

Step 5. A mixture of tert-butyl (2R)-2-[[tert-butyl(diphenyl)silyl]oxymethyl]-3-oxo-piperidine-1-carboxylate (350 mg, 0.748 mmol, 1 eq) in THE (4 mL) was degassed and purged with N₂ 3 times at −40° C., and then, MeMgBr (2.5 M, 0.898 mL, 3 eq) was added. The mixture was stirred at 25° C. for 1 hour under N₂ atmosphere. On completion, the mixture was quenched with sat. NH₄Cl aqueous (0.5 mL) and extracted with EtOAc (5 mL×3), the combined organic phase was dried over anhydrous sodium sulfate, filtered and concentrated to give tert-butyl (2R)-2-[[tert-butyl(diphenyl)silyl]oxymethyl]-3-hydroxy-3-methyl-piperidine-1-carboxylate (160 mg, 0.297 mmol, 40% yield, 90% purity) as a white solid. ¹H NMR (400 MHz, DMSO-d) δ=7.66-7.59 (m, 4H), 7.48-7.39 (m, 6H), 4.30-4.13 (m, 1H), 3.96-3.87 (m, 2H), 3.87-3.76 (m, 1H), 2.56 (s, 1H), 1.44 (s, 2H), 1.39 (s, 9H), 1.37-1.29 (m, 2H), 1.16-1.12 (m, 3H), 0.97 (d, J=6.0 Hz, 9H).

Step 6. To a solution of tert-butyl (2R)-2-[[tert-butyl(diphenyl)silyl]oxymethyl]-3-hydroxy-3-methyl-piperidine-1-carboxylate (360 mg, 0.744 mmol, 1 eq) in DMSO (1 mL) was added CsF (678 mg, 4.47 mmol, 6 eq). The mixture was stirred at 40° C. for 3 h. On completion, acetonitrile (3 mL) was added to the reaction solution, which was filtered and concentrated under reduced pressure to obtain the residue. The residue was purified by prep-TLC (SiO₂, PE:EtOAc=0:1) to give tert-butyl (2R)-3-hydroxy-2-(hydroxymethyl)-3-methyl-piperidine-1-carboxylate (160 mg, 0.652 mmol, 88% yield) as a white solid. ¹H NMR (400 MHz, CDCl₃) δ=4.19-4.09 (m, 1H), 3.96 (s, 1H), 3.86-3.54 (m, 2H), 3.09-2.82 (m, 1H), 1.86-1.79 (m, 1H), 1.67 (d, J=4.0 Hz, 1H), 1.60 (d, J=4.4 Hz, 1H), 1.57 (d, J=3.6 Hz, 1H), 1.47 (s, 9H), 1.36 (s, 3H).

Step 7. To a solution of tert-butyl (2R)-3-hydroxy-2-(hydroxymethyl)-3-methyl-piperidine-1-carboxylate (160 mg, 0.652 mmol, 1 eq) in DCM (0.5 mL) was added HCl/dioxane (4 M, 0.1 mL). The mixture was stirred at 25° C. for 0.5 h. On completion, the mixture was concentrated in vacuo to give (2R)-2-(hydroxymethyl)-3-methyl-piperidin-3-ol (94.0 mg, 0.647 mmol, 99% yield) as a white solid. The crude product was used into the next step without further purification (I-2-18).

I-2-19 is commercially available.

### Preparation of ((3S,5R)-5-methylmorpholin-3-yl)methanol (I-2-20)

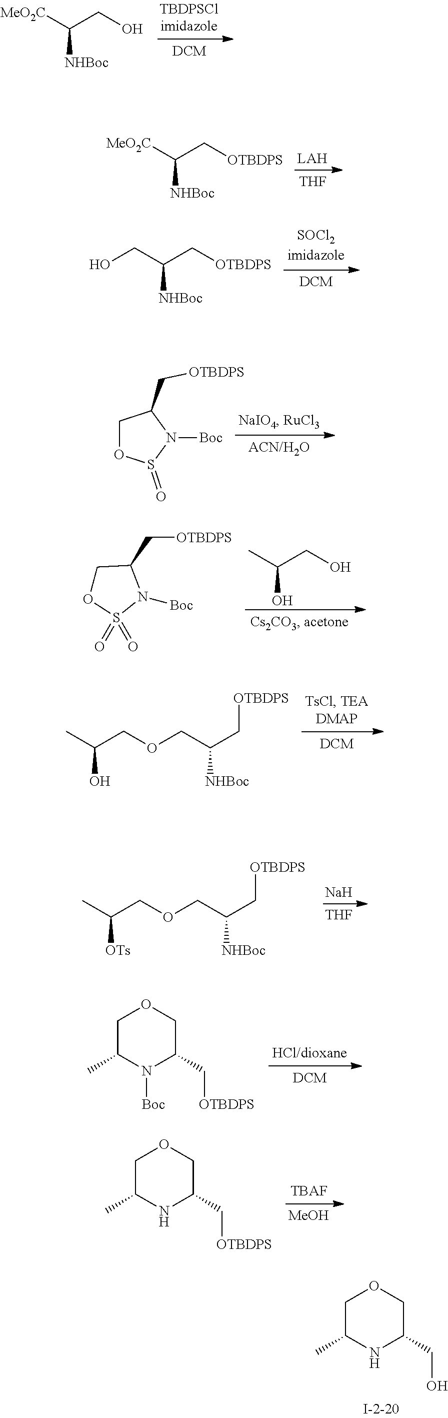

Step 1. To a solution of methyl (tert-butoxycarbonyl)-D-serinate (50.0 g, 228 mmol, 1 eq) in DCM (500 mL) was added imidazole (31.0 g, 456 mmol, 2 eq) and TBDPSCl (75.2 g, 273 mmol, 70.0 mL, 1.2 eq). The mixture was stirred at 25° C. for 3 hours. On completion, the reaction mixture was diluted with H₂O (500 mL) and extracted with DCM (200 mL*5). The combined organic layers were washed with brine (300 mL), dried over Na₂SO₄, filtered, and concentrated in vacuo to give methyl N-(tert-butoxycarbonyl)-O-(tert-butyldiphenylsilyl)-D-serinate (100 g, 218 mmol, 95% yield) as yellow oil. LCMS: (M+23: 480.2).

Step 2. A mixture of methyl N-(tert-butoxycarbonyl)-O-(tert-butyldiphenylsilyl)-D-serinate (50.0 g, 109 mmol, 1 eq) in THF (500 mL) was degassed and purged with N₂ 3 times, LiAlH₄ (2.5 M, 48 mL, 1.1 eq) was added at 0° C., and then, the mixture was stirred at 0° C. for 1 hour under N₂ atmosphere. On completion, the mixture was added with H₂O (6 mL), 15% NaOH (6 mL) and H₂O (18 mL). And then diluted with H₂O (500 mL) and extracted with EtOAc [600 mL (200 mL*3)]. The combined organic layers were dried over Na₂SO₄, filtered and concentrated in vacuo to give tert-butyl (S)-(1-((tert-butyldiphenylsilyl)oxy)-3-hydroxypropan-2-yl)carbamate (25.0 g, 58.1 mmol, 53% yield) as canary yellow oil. LCMS: (M+23: 452.2).

Step 3. To a solution of tert-butyl (S)-(1-((tert-butyldiphenylsilyl)oxy)-3-hydroxypropan-2-yl)carbamate (54 g, 125 mmol, 1 eq) in DCM (540 mL) was added SOCl₂ (29.9 g, 251 mmol, 18.5 mL, 2 eq) and Imidazole (51.3 g, 754 mmol, 6 eq). The mixture was stirred at 25° C. for 3 hours. On completion, the reaction mixture was quenched by addition H₂O (500 mL) at 0° C., and then extracted with DCM (200 mL*5). The combined organic layers were washed with brine (200 mL), dried over Na₂SO₄, filtered and concentrated in vacuo to give tert-butyl (4R)-4-(((tert-butyldiphenylsilyl)oxy)methyl)-1,2,3-oxathiazolidine-3-carboxylate 2-oxide (20.0 g, 42.0 mmol, 33% yield) as a yellow oil. LCMS: (M+23: 498.1).

Step 4. To a solution of tert-butyl (4R)-4-(((tert-butyldiphenylsilyl)oxy)methyl)-1,2,3-oxathiazolidine-3-carboxylate 2-oxide (10.0 g, 21.0 mmol, 1 eq) in Acetonitrile (50 mL) and H₂O (50 mL) was added RuCl₃ (436 mg, 2.10 mmol, 0.1 eq) and NaIO₄ (6.30 g, 29.4 mmol, 1.4 eq). The mixture was stirred at 25° C. for 16 hours. On completion, the reaction mixture was quenched by addition of ice-water (50 mL) at 0° C., and then diluted with ice water (50 mL) and extracted with EtOAc (80 mL*3). The combined organic layers were washed with brine (50 mL), dried over Na₂SO₄, filtered and concentrated under reduced pressure to give tert-butyl (R)-4-(((tert-butyldiphenylsilyl)oxy)methyl)-1,2,3-oxathiazolidine-3-carboxylate 2,2-dioxide (8.10 g, 16.4 mmol, 78% yield) as a yellow oil. ¹H NMR (400 MHz, DMSO-d₆) δ 7.66-7.59 (m, 4H), 7.52-7.41 (m, 6H), 4.47-4.10 (m, 2H), 3.94-3.85 (m, 1H), 3.79-3.53 (m, 2H), 1.68 (t, J=1.2 Hz, 1H), 1.51-1.29 (m, 7H), 1.09-0.80 (m, 10H); LCMS: (M+23: 514.1).

Step 5. To a solution of Cs₂CO₃ (12.7 g, 39.0 mmol, 2.4 eq) and molecular sieve in Acetone (3 mL) was added tert-butyl (R)-4-(((tert-butyldiphenylsilyl)oxy)methyl)-1,2,3-oxathiazolidine-3-carboxylate 2,2-dioxide (8 g, 16.2 mmol, 1 eq) and (2S)-propane-1,2-diol (2.97 g, 39.0 mmol, 3.0 mL, 2.4 eq). The mixture was stirred at 25° C. for 16 hours. On completion, the reaction mixture was then diluted with H₂O (500 mL) and extracted with EtOAc (200 mL*3). The combined organic layers were washed with brine (200 mL), dried over Na₂SO₄, filtered and concentrated in vacuo to give tert-butyl ((R)-1-((tert-butyldiphenylsilyl)oxy)-3-((S)-2-hydroxypropoxy)propan-2-yl)carbamate (3.1 g, 6.36 mmol, 39% yield) as yellow oil. LCMS: (M+23: 510.2).

Step 6. To a solution of tert-butyl ((R)-1-((tert-butyldiphenylsilyl)oxy)-3-((S)-2-hydroxypropoxy)propan-2-yl)carbamate (3.10 g, 6.36 mmol, 1 eq) in DCM (15 mL) was added Triethylamine (2.12 g, 20.9 mmol, 3.3 eq) and DMAP (466 mg, 3.81 mmol, 0.6 eq), then 4-methylbenzenesulfonyl chloride (2.42 g, 12.7 mmol, 2 eq) was added. The mixture was stirred at 25° C. for 16 hours. On completion, the reaction mixture was diluted with H₂O (300 mL) and extracted with DCM (100 mL*3). The combined organic layers were washed with brine (200 mL), dried over Na₂SO₄, filtered in and concentrated vacuo to give (S)-1-((R)-2-((tert-butoxycarbonyl)amino)-3-((tert-butyldiphenylsilyl)oxy)propoxy)propan-2-yl 4-methylbenzenesulfonate (2.50 g, 3.89 mmol, 61% yield) as canary yellow oil. LCMS: (M+1: 642.2).

Step 7. To a solution of (S)-1-((R)-2-((tert-butoxycarbonyl)amino)-3-((tert-butyldiphenylsilyl)oxy)propoxy)propan-2-yl 4-methylbenzenesulfonate (2.50 g, 3.89 mmol, 1 eq) in THF (30 mL) was added NaH (623 mg, 15.5 mmol, 60% purity, 4 eq). The mixture was stirred at 0° C. for 1 hour. On completion, the reaction mixture was quenched by addition NH₄Cl (5 mL) at 0° C., and then diluted with H₂O (100 mL) and extracted with EtOAc (30 mL*3). The combined organic layers were washed with brine (20 mL), dried over Na₂SO₄, filtered and concentrated in vacuo to give tert-butyl (3R,5R)-3-(((tert-butyldiphenylsilyl)oxy)methyl)-5-methylmorpholine-4-carboxylate (1.25 g, 2.66 mmol, 68% yield) as an orange solid. LCMS: (M-100+1:370.3).

Step 8. To a solution of tert-butyl (3R,5R)-3-(((tert-butyldiphenylsilyl)oxy)methyl)-5-methylmorpholine-4-carboxylate (600 mg, 1.28 mmol, 1 eq) in DCM (4 mL) was added HCl/dioxane (4 M, 1.50 mL, 4.70 eq). The mixture was stirred at 25° C. for 0.5 hour. On completion, the reaction mixture was concentrated in vacuo to give (3R,5R)-3-(((tert-butyldiphenylsilyl)oxy)methyl)-5-methylmorpholine (450 mg, 1.22 mmol, 95% yield) as a yellow solid. LCMS: (M+1: 370.5).

Step 9. To a solution of (3R,5R)-3-(((tert-butyldiphenylsilyl)oxy)methyl)-5-methylmorpholine (450 mg, 1.22 mmol, 1 eq) in MeOH (12 mL) was added tetrabutylammonium fluoride trihydrate (1 M, 4.87 mL, 4 eq). The mixture was stirred at 25° C. for 1 hour. On completion, the reaction mixture was concentrated in vacuo to give ((3S,5R)-5-methylmorpholin-3-yl)methanol (150 mg, 1.14 mmol, 93% yield) as colorless oil (I-2-20). LCMS: (M+1: 131.9).

### Preparation of 2-(benzylamino)-3-((tert-butyldiphenylsilyl)oxy)propan-1-ol (I-2-21)

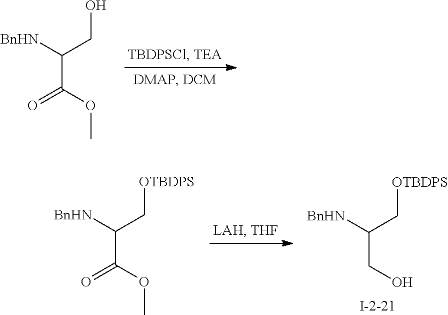

Step 1. To a mixture of methyl 2-(benzylamino)-3-hydroxy-propanoate (5.00 g, 23.9 mmol, 1 eq) in DCM (50 mL) was added Triethylamine (2.90 g, 28.7 mmol, 1.2 eq) and DMAP (146 mg, 1.19 mmol, 0.05 eq), and then TBDPSCl (6.90 g, 25.1 mmol, 1.05 eq) was added at 0° C. The mixture was stirred at 25° C. for 12 h. On completion, the mixture was quenched with dilute citric acid (150 mL) and extracted with DCM (50 mL×3), the combined organic phase was dried over anhydrous Na₂SO₄, filtered and the filtrate was concentrated to give a residue. The residue was purified by column chromatography (SiO₂, PE:THF=5:1) to give methyl 2-(benzylamino)-3-[tert-butyl(diphenyl)silyl]oxy-propanoate (10.3 g, crude) as a white oil. LCMS: (M+1: 448.7).

Step 2. To a solution of methyl 2-(benzylamino)-3-[tert-butyl(diphenyl)silyl]oxy-propanoate (2.95 g, 6.59 mmol, 1 eq) in THF (30 mL) was added LAH (375 mg, 9.89 mmol, 1.5 eq) at 0° C. The mixture was stirred at 25° C. for 2 h. On completion, the mixture was quenched with water (0.375 mL), 15% NaOH solution (0.375 mL), water (1 mL), then the mixture was dried over Na₂SO₄, filtered and the filtrate was concentrated to give a residue. The residue was purified by column chromatography (SiO₂, PE:THF=5:1) to give 2-(benzylamino)-3-[tert-butyl(diphenyl)silyl]oxy-propan-1-ol (684 mg, crude) as a white oil (I-2-21). LCMS: (M+1: 420.1).

### Preparation of (S)-2-(benzylamino)butan-1-ol (I-2-22)

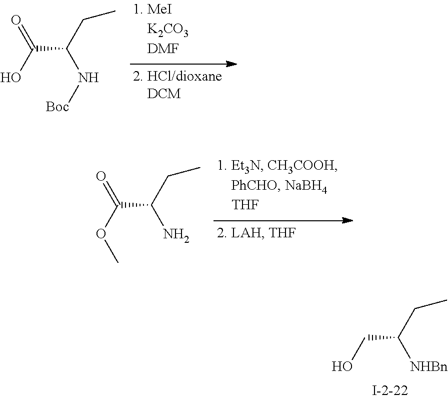

Step 1. To a solution of (2S)-2-(tert-butoxycarbonylamino)butanoic acid (4.50 g, 22.1 mmol, 1 eq) in DMF (50 mL) was added Mel (5.03 g, 35.4 mmol, 1.6 eq) and K₂CO₃ (9.18 g, 66.4 mmol, 3 eq). The mixture was stirred at 25° C. for 12 h. On completion, the mixture was quenched with water (100 mL) and extracted with ethyl acetate (50 mL×3), the combined organic phase was dried over anhydrous sodium sulfate, concentrated to give compound methyl (2S)-2-(tert-butoxycarbonylamino)butanoate (4.80 g, 22.1 mmol, 99% yield) as a yellow solid. ¹H NMR (400 MHz, DMSO-d₆) δ=7.22 (br d, J=7.6 Hz, 1H), 3.87 (dt, J=5.6, 8.0 Hz, 1H), 3.61 (s, 3H), 1.71-1.52 (m, 2H), 1.41-1.30 (m, 9H), 0.86 (t, J=7.2 Hz, 3H).

To a solution of methyl (2S)-2-(tert-butoxycarbonylamino)butanoate (1.50 g, 6.90 mmol, 1 eq) in DCM (10 mL) was added HCl/dioxane (4 M, 2 mL, 1 eq). The mixture was stirred at 25° C. for 1 h. On completion, the mixture was concentrated to give methyl (2S)-2-aminobutanoate (800 mg, 6.83 mmol, 98% yield) as a yellow solid. LCMS: (M+1: 118.0).

Step 2. To a solution of methyl (2S)-2-aminobutanoate (800 mg, 6.83 mmol, 1 eq) in THE (5 mL) was added Triethylamine (6.83 mmol, 1 mL, 1 eq) to adjust pH=7-8, then CH₃COOH (68.3 mmol, 4 mL, 10 eq), benzaldehyde (725 mg, 6.83 mmol, 1 eq), NaBH₄ (517 mg, 13.6 mmol, 2 eq) were added into the mixture. The mixture was stirred at 25° C. for 1 h. On completion, the mixture was quenched with water (20 mL) and extracted with ethyl acetate (25 mL×3), the combined organic phase was dried over anhydrous sodium sulfate, concentrated to give a residue. The residue was purified by column chromatography (SiO₂, petroleum ether/ethyl acetate=1:0 to 5:1) to give compound methyl (2S)-2-(benzylamino)butanoate (870 mg, 4.20 mmol, 61% yield) as a yellow solid. ¹H NMR (400 MHz, DMSO-d₆) δ=7.35-7.18 (m, 5H), 3.73 (d, J=13.6 Hz, 1H), 3.63 (s, 3H), 3.54 (d, J=13.6 Hz, 1H), 3.09 (t, J=6.8 Hz, 1H), 1.91 (s, 1H), 1.58 (dquin, J=3.2, 7.2 Hz, 2H), 0.85 (t, J=7.2 Hz, 3H); LCMS: (M+1: 208.0).

To a solution of methyl (2S)-2-(benzylamino)butanoate (870 mg, 4.20 mmol, 1 eq) in THE (10 mL) was added LiAlH₄ (2.5 M, 1.7 mL, 1 eq). The mixture was stirred at 0° C. for 1 h. On completion, the mixture was quenched with water (5 mL), 15% aq. NaOH (5 mL), water (15 mL), and extracted with ethyl acetate (30 mL×3), the combined organic layers were washed with NaCl solution (30 mL×3), dried over Na₂SO₄, filtered and concentrated under reduced pressure to give (2S)-2-(benzylamino)butan-1-ol (700 mg, 3.90 mmol, 93% yield) as a yellow solid (I-2-22). ¹H NMR (400 MHz, DMSO-d₆) δ=7.35-7.26 (m, 5H), 7.24-7.18 (m, 1H), 4.51-4.39 (m, 1H), 3.70 (d, J=2.4 Hz, 2H), 3.41-3.36 (m, 1H), 3.31-3.25 (m, 1H), 2.43-2.36 (m, 1H), 1.42-1.33 (m, 2H), 0.83 (t, J=7.6 Hz, 3H); LCMS: (M+1: 180.2).

### Preparation of (1-(methylamino)cyclopropyl)methanol (I-2-23)

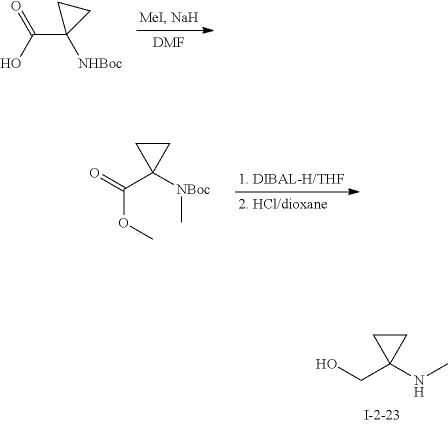

Step 1. A mixture of 1-(tert-butoxycarbonylamino)cyclopropanecarboxylic acid (5.00 g, 24.9 mmol, 1 eq), NaH (2.98 g, 74.5 mmol, 60% purity, 3 eq) in DMF (50 mL) at 0° C. for 0.5 h, and then add Mel (10.6 g, 74.6 mmol, 4.64 mL, 3 eq). The mixture was stirred at 25° C. for 16 h. On completion, the reaction mixture was quenched by addition NH₄Cl (50 ml) at 0° C., and then diluted with H₂O (80 mL) and extracted with EtOAc (40 mL*3). The combined organic layers were washed with H₂O (100 mL*3), dried over Na₂SO₄, filtered, and concentrated under reduced pressure to give a residue. The residue was purified by column chromatography on silica gel (12 g silica gel, EtOAc in Petroleum ether from 0% to 100%) to give methyl 1-[tert-butoxycarbonyl(methyl)amino]cyclopropanecarboxylate (5.47 g, 22.3 mmol, 90% yield, 94% purity) as a colorless oil. ¹H NMR (400 MHz, CDCl₃) δ=3.75-3.67 (m, 3H), 3.04 (s, 3H), 1.84-1.57 (m, 2H), 1.48-1.40 (m, 9H), 1.30-1.08 (m, 2H).

Step 2. To a solution of methyl 1-[tert-butoxycarbonyl (methyl) amino]cyclopropanecarboxylate (2.00 g, 8.72 mmol, 1 eq) in THF (30 mL) and degassed and purged with N₂ 3 times. Then added DIBAL-H (1 M, 26.1 mL, 3 eq) at 0° C., the mixture was stirred at 25° C. for 2 h. On completion, the reaction mixture was quenched by sat. NH₄Cl (30 mL) at 0° C., and then diluted with H₂O (40 mL) and extracted with EtOAc (20 mL*3). The combined organic layers were washed with H₂O (20 mL*3), dried over Na₂SO₄, filtered, and concentrated under reduced pressure to give a residue. The residue was purified by column chromatography on silica gel (4 g silica gel, EtOAc in Petroleum ether from 0% to 100%) to give tert-butyl N-[1-(hydroxymethyl)cyclopropyl]-N-methyl-carbamate (1.20 g, 5.96 mmol, 68% yield) as a white solid. ¹H NMR (400 MHz, CDCl₃) δ=3.57 (s, 2H), 2.88 (s, 3H), 1.46 (s, 9H), 0.91-0.82 (m, 4H).

To a solution of tert-butyl N-[1-(hydroxymethyl)cyclopropyl]-N-methyl-carbamate (800 mg, 3.97 mmol, 1 eq) in DCM (8 mL) was added HCl/dioxane (4 M, 8 mL, 8.05 eq) at 0° C. The mixture was stirred at 25° C. for 1 h. On completion, the reaction mixture was concentrated in vacuum to give [1-(methylamino) cyclopropyl]methanol (400 mg, crude) as a colorless oil (I-2-23). ¹H NMR (400 MHz, DMSO-d₆) δ=9.30 (s, 1H), 3.55-3.52 (m, 2H), 2.58-2.52 (m, 3H), 1.10-1.03 (m, 2H), 0.78-0.70 (m, 2H). LCMS: (M+1=176.1).

### Preparation of (1-(benzylamino)cyclopropyl)methanol (I-2-24)

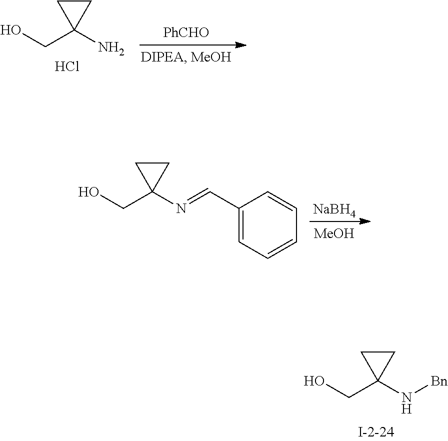

Step 1: To a solution of (1-aminocyclopropyl)methanol (2.00 g, 16.2 mmol, 1 eq, HCl) in MeOH (20 mL) was added benzaldehyde (1.72 g, 16.2 mmol, 1.64 mL, 1 eq) and DIPEA (2.09 g, 16.2 mmol, 2.82 ml, 1 eq). The mixture was stirred at 25° C. for 1 hr. On completion, 1-(benzylideneamino)cyclopropyl)methanol (2.60 g, crude in MeOH) was obtained as a black liquid. LCMS: (M+1=176.1).

To a solution of 1-(benzylideneamino)cyclopropyl)methanol (2.60 g, crude in MeOH, 1 eq) in MeOH (20 mL) was added NaBH₄ (1.68 g, 44.5 mmol, 3 eq) at 0° C. The mixture was stirred at 25° C. for 1.5 hr. On completion, the mixture was quenched with MeOH (10 mL) until no gas formed and concentrated under reduced pressure to give a residue. The residue was purified by column chromatography (SiO₂, Dichloromethane/Methanol=30/1 to 10/1) to give [1-(benzylamino)cyclopropyl]methanol (1.60 g, 9.03 mmol, 61% yield) as a white solid (I-2-24). ¹H NMR (400 MHz, DMSO-d₆) δ=7.41-7.13 (m, 5H), 4.60 (br s, 1H), 3.75 (s, 2H), 3.41 (s, 2H), 2.40 (br s, 1H), 0.54-0.35 (m, 4H). LCMS: (M+1=178.2).

### Preparation of (S)-2-(methylamino)butan-1-ol (I-2-25)

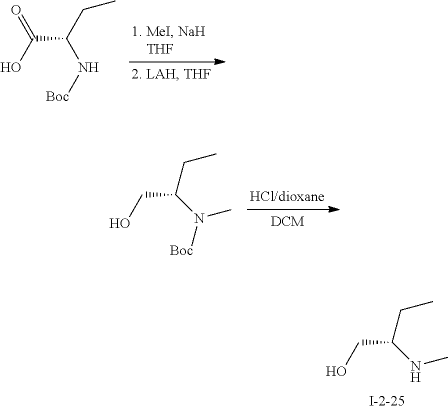

Step 1. To a solution of (2S)-2-(tert-butoxycarbonylamino)butanoic acid (4.00 g, 19.6 mmol, 1 eq) in THF (40 mL) was added NaH (1.57 g, 39.3 mmol, 60% purity, 2 eq) at 0° C. And then, the mixture was added Mel (6.98 g, 49.2 mmol, 2.5 eq) dropwise at 0° C. The mixture was stirred at 25° C. for 1 hour. On completion, the mixture was quenched with water (30 mL) and extracted with ethyl acetate (20 mL×3), the combined organic phase was dried over anhydrous sodium sulfate, filtered and concentrated to give methyl (2S)-2-[tert-butoxycarbonyl(methyl) amino]butanoate (4.5 g, 19.4 mmol, 98% yield) as yellow oil. ¹H NMR (400 MHz, CDCl₃) δ=4.33-4.17 (m, 1H), 3.68 (s, 3H), 2.83-2.72 (m, 3H), 1.82-1.56 (m, 2H), 1.42 (s, 9H), 0.94-0.89 (m, 3H).

To a solution of methyl (2S)-2-[tert-butoxycarbonyl(methyl)amino]butanoate (4.00 g, 17.2 mmol, 1 eq) in THF (50 mL) was added LAH (2.5 M, 8.30 mL, 1.2 eq). The mixture was stirred at 0° C. for 2 hours. On completion, the mixture was quenched with water (1 mL), 15% NaOH (1 mL) and water (3 mL), the mixture was dried over anhydrous sodium sulfate, filtered, and concentrated to give a residue. The residue was purified by column chromatography (SiO₂, Petroleum ether/Ethyl acetate=1:0 to 0:1) to give tert-butyl N-[(1S)-1-(hydroxymethyl)propyl]-N-methyl-carbamate (2.00 g, 9.84 mmol, 56% yield) as yellow oil. ¹H NMR (400 MHz, CDCl₃) δ=7.25-7.16 (m, 1H), 3.99-3.86 (m, 1H), 3.65-3.53 (m, 1H), 3.52-3.45 (m, 2H), 2.66 (s, 3H), 2.54 (br s, 1H), 1.39 (s, 9H), 0.88 (t, J=7.6 Hz, 1H), 0.82 (t, J=7.2 Hz, 3H).

Step 2. To a solution of tert-butyl N-[(1S)-1-(hydroxymethyl)propyl]-N-methyl-carbamate (1 g, 4.92 mmol, 1 eq) in DCM (10 mL) was added HCl/dioxane (4 M, 1 mL, 0.5 eq). The mixture was stirred at 25° C. for 1 hour. On completion, the mixture was concentrated to give compound (2S)-2-(methylamino)butan-1-ol (500 mg, 4.85 mmol, 98% yield) as yellow oil (I-2-25).

### Preparation of (S)-(5,5-difluoropiperidin-2-yl)methanol (I-2-26)

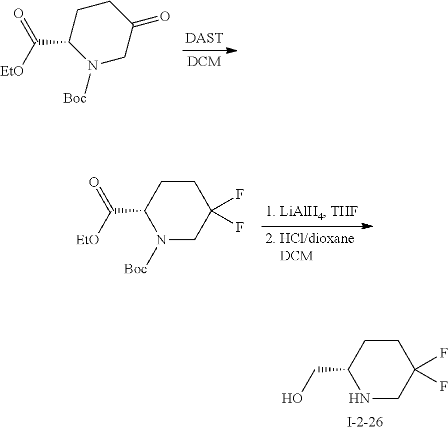

Step 1. To a solution of 01-tert-butyl 02-methyl (S)-5-oxopiperidine-1,2-dicarboxylate (4.00 g, 15.5 mmol, 1 eq) in DCM (40 mL) was added DAST (15.0 g, 93.2 mmol, 6 eq). The mixture was stirred at 25° C. for 4 h. On completion, the reaction mixture was quenched with H₂O (20 mL) at 0° C. and extracted with DCM (10 mL*3). The combined organic layers were washed with brine (30 mL*2), dried over anhydrous sodium sulfate, filtered, and concentrated to give 01-tert-butyl 02-methyl (S)-5,5-difluoropiperidine-1,2-dicarboxylate (4.00 g, 9.45 mmol, 61% yield, 66% purity) as yellow oil. ¹H NMR (400 MHz, DMSO-d₆)=4.88-4.69 (m, 1H), 4.17 (d, J=6.8 Hz, 2H), 4.09-3.99 (m, 1H), 3.28-3.00 (m, 1H), 2.22-2.08 (m, 2H), 1.95-1.84 (m, 1H), 1.83-1.67 (m, 1H), 1.40 (d, J=12.4 Hz, 9H), 1.21 (t, J=7.2 Hz, 3H).

Step 2. A mixture of O1-tert-butyl O2-ethyl (S)-5,5-difluoropiperidine-1,2-dicarboxylate (3.5 g, 11.9 mmol, 1 eq) in THE (5 mL) was degassed and purged with N₂ 3 times, LiAlH₄ (2.5 M, 6.0 mL, 1.2 eq) was added at 0° C., and then the mixture was stirred at 0° C. for 2 h under N₂ atmosphere. On completion, the reaction mixture was quenched by addition H₂O (0.5 mL), 15% NaOH (0.5 mL) and H₂O (1.5 mL) at 0° C., and then filtered and concentrated under reduced pressure to give a residue. The residue was diluted with H₂O (10 mL) and extracted with EtOAc 30 mL (10 mL*3). The combined organic layers were washed with brine 10 mL (5 mL*2), dried over anhydrous sodium sulfate, filtered, and concentrated. The residue was purified by column chromatography (SiO₂, Petroleum ether/Ethyl acetate=100/1 to 5/1) to give tert-butyl (2S)-5,5-difluoro-2-(hydroxymethyl)piperidine-1-carboxylate (700 mg, 2.70 mmol, 23% yield, 97% purity) as a yellow solid. ¹H NMR (400 MHz, DMSO-d6) δ=4.83 (t, J=5.6 Hz, 1H), 4.12-3.97 (m, 2H), 3.56-3.43 (m, 2H), 3.28-3.10 (m, 1H), 2.15-2.01 (m, 1H), 2.00-1.92 (m, 1H), 1.89-1.80 (m, 1H), 1.69-1.59 (m, 1H), 1.42-1.38 (m, 9H). LCMS: (M+1-100: 152.1).

To a solution of tert-butyl (2S)-5,5-difluoro-2-(hydroxymethyl)piperidine-1-carboxylate (400 mg, 1.59 mmol, 1 eq) in DCM (2 mL) was added HCl/dioxane (4 M, 1 mL). The mixture was stirred at 25° C. for 1 h. On completion, the mixture was concentrated under reduced pressure to give [(S)-5,5-difluoro-2-piperidyl]methanol (240 mg, 1.28 mmol, 81% yield, HCl salt) as yellow oil (I-2-26).

### Preparation of (S)-2-(benzylamino)-3-((tert-butyldiphenylsilyl)oxy)propan-1-ol (I-2-27)

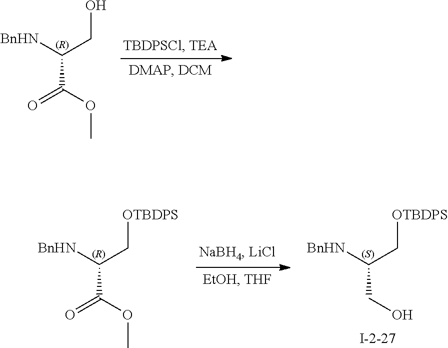

Step 1. To a solution of methyl (2R)-2-(benzylamino)-3-hydroxy-propanoate (5.00 g, 23.9 mmol, 1 eq) in DCM (50 mL) was added Triethylamine (2.90 g, 28.7 mmol, 1.2 eq) and DMAP (146 mg, 1.19 mmol, 0.05 eq), then TBDPSCl (6.90 g, 25.1 mmol, 1.05 eq) was added at 0° C. The mixture was stirred at 25° C. for 1 h. On completion, the mixture was quenched with dilute citric acid (150 mL) and extracted with DCM (50 mL×3), the combined organic phase was dried over anhydrous Na₂SO₄, filtered and the filtrate was concentrated to give a residue. The residue was purified by column chromatography (SiO₂, PE:THF=5:1) to give methyl (2R)-2-(benzylamino)-3-[tert-butyl(diphenyl) silyl]oxy-propanoate (9.19 g, 20.5 mmol, 86% yield) as a white oil. LCMS: (M+1: 448.4).

Step 2. To a solution of methyl (2R)-2-(benzylamino)-3-[tert-butyl(diphenyl)silyl]oxy-propanoate (2.00 g, 4.47 mmol, 1 eq) in THF (18 mL) and EtOH (2 mL) was added LiCl (1.23 g, 29.0 mmol, 6.5 eq) and NaBH₄ (1.69 g, 44.7 mmol, 10 eq) at 0° C. The mixture was stirred at 25° C. for 16 h. On completion, the mixture was diluted with MeOH (60 mL) and concentrated to give a residue. The residue was purified by column chromatography (SiO₂, PE:THF=5:1) to give (2S)-2-(benzylamino)-3-[tert-butyl(diphenyl)silyl]oxy-propan-1-ol (735 mg, crude) as a white oil (I-2-27). LCMS: (M+1: 420.6).

### Preparation of (1R,2S)-2-(benzylamino)cyclopentan-1-ol (I-2-28)

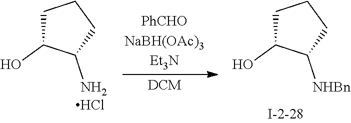

To a suspension of (1R,2S)-2-aminocyclopentanol hydrochloride (2 g, 14.53 mmol) in DCM (70 mL) was added Triethylamine (1.76 g, 17.44 mmol, 2.43 mL), followed by Benzaldehyde (1.54 g, 14.53 mmol, 1.48 mL). The suspension was stirred for 30 minutes, before adding Sodium triacetoxyborohydride (3.08 g, 14.53 mmol). The mixture was then stirred for 3 h at 23° C. The reaction mixture was diluted with satd. aq. NaHCO₃ (70 mL) and extracted with CH₂Cl₂ (3×150 mL). The combined organic extracts were dried over anhydrous Na₂SO₄ and concentrated to give a residue. Purification by silica gel chromatography (Automated system, 120 g silica, Hex/EA=1/0 to 0/1) afforded (1R,2S)-2-(benzylamino)cyclopentanol (1.01 g, 5.28 mmol, 36.33% yield) as a yellow oil (I-2-28). LCMS: (M+1: 192.2).

Intermediate I-2-29 is commercially available.

### Preparation of 6-(hydroxymethyl)piperidine-3-carboxamide (I-2-30)

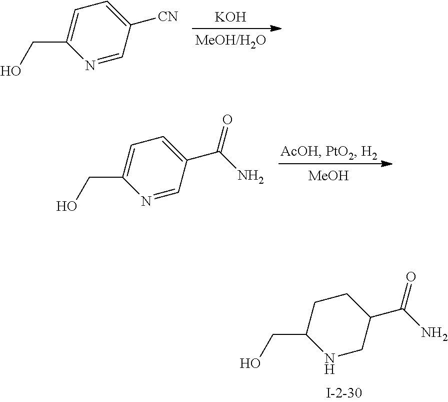

Step 1. To a solution of 6-(hydroxymethyl)pyridine-3-carbonitrile (2.00 g, 14.9 mmol, 1 eq) in MeOH (20 mL) and H₂O (2 mL) was added KOH (2.51 g, 44.7 mmol, 3 eq). The mixture was stirred at 50° C. for 2 h. On completion, the mixture was concentrated in vacuum to give crude. The residue was purified by combi flash (40 g silica gel column, MeOH in DCM from 0% to 100%). 6-(hydroxymethyl)pyridine-3-carboxamide (1.00 g, 6.57 mmol, 44% yield) was obtained as green solid. ¹H NMR (400 MHz, DMSO-d₆) δ=9.01-8.83 (m, 1H), 8.27-8.17 (m, 1H), 8.13-8.01 (m, 1H), 7.63-7.41 (m, 2H), 5.58-5.43 (m, 1H), 4.66-4.50 (m, 2H).

Step 2. To a solution of 6-(hydroxymethyl)pyridine-3-carboxamide (500 mg, 3.29 mmol, 1 eq), PtO₂ (50.0 mg, 0.022 mmol, 10% purity, 0.007 eq) and AcOH (98.7 mg, 1.64 mmol, 0.5 eq) in MeOH (5 mL) was stirred at 50° C. for 16 h under H₂ (50 psi). On completion, the mixture was filtered, and the filtrate was concentrated in vacuum to give crude. The residue was purified by combi flash (12 g silica gel, MeOH in DCM from 0% to 100%) to give 6-(hydroxymethyl)piperidine-3-carboxamide (490 mg, 3.10 mmol, 94% yield) as yellow solid (I-2-30). ¹H NMR (400 MHz, MeOD-d₄) δ=3.68-3.60 (m, 1H), 3.60-3.52 (m, 1H), 3.50-3.39 (m, 1H), 3.37-3.33 (m, 4H), 3.04-2.93 (m, 2H), 2.87-2.74 (m, 1H), 2.72-2.63 (m, 1H), 2.10-1.98 (m, 1H), 1.73-1.62 (m, 2H).

Intermediates I-2-31 and I-2-32 are commercially available.

Intermediate I-2-33 was prepared following the same synthetic route as the preparation of I-2-27, using (2S)-2-(benzylamino)-3-hydroxy-propanoate as the starting material.

### Preparation of 2-(cyclopropylamino)propan-1-ol (I-2-34)

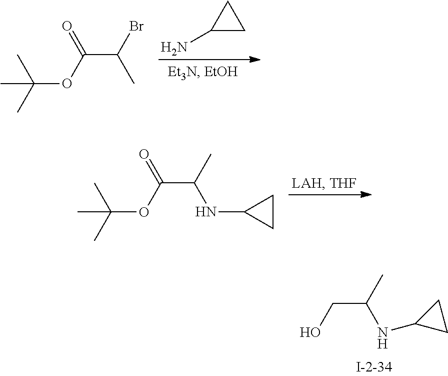

Step 1. The mixture of cyclopropanamine (1.64 g, 28.7 mmol, 2 eq) and Triethylamine (1.81 g, 17.9 mmol, 1.25 eq) in EtOH (20 mL) was added a solution of tert-butyl 2-bromopropanoate (3.00 g, 14.3 mmol, 1 eq) in EtOH (10 mL) dropwise at 0° C. The mixture was stirred at 25° C. for 24 h. On completion, the mixture was concentrated to give a residue. The residue was purified by column chromatography (SiO₂, PE:THF=1:0 to 94:6) to give tert-butyl 2-(cyclopropylamino)propanoate (1.10 g, 5.94 mmol, 41% yield) as a colorless oil. ¹H NMR (400 MHz, CDCl₃) δ=3.37-3.29 (m, 1H), 2.16-2.08 (m, 1H), 1.49 (s, 9H), 1.25 (d, J=7.2 Hz, 3H), 0.46-0.41 (m, 3H), 0.36-0.30 (m, 1H).

Step 2. To a solution of tert-butyl 2-(cyclopropylamino)propanoate (1.05 g, 5.67 mmol, 1 eq) in THF (11 mL) was added LAH (2.5 M, 3.40 mL, 1.5 eq) at 0° C. The mixture was stirred at 0° C. for 2 h. On completion, the mixture was quenched with water (0.3 mL), 15% NaOH (0.3 mL) and water (1.2 mL) successively, dried over Na₂SO₄, filtered and concentrated to give 2-(cyclopropylamino)propan-1-ol (850 mg, crude) as a colorless oil (I-2-34). ¹H NMR (400 MHz, CDCl₃) δ=3.62-3.57 (m, 1H), 3.26-3.19 (m, 1H), 2.95-2.86 (m, 1H), 2.22-2.17 (m, 1H), 1.10 (d, J=6.4 Hz, 3H), 0.50-0.36 (m, 3H), 0.33-0.26 (m, 1H).

Intermediate I-2-35 is commercially available.

### Preparation of (S)-2-(isopropylamino)propan-1-ol (I-2-36)

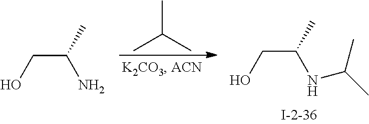

To a solution of (2S)-2-aminopropan-1-ol (500 mg, 6.66 mmol, 0.518 mL, 1 eq), 2-iodopropane (1.70 g, 9.99 mmol, 0.996 mL, 1.5 eq) in ACN (5 mL) was added K₂CO₃ (920 mg, 6.66 mmol, 1 eq). The mixture was stirred at 80° C. for 2 hr. On completion, the mixture was filtered and concentrated to give (2S)-2-(isopropylamino)propan-1-ol (780 mg, crude) as colorless oil (I-2-36).

### Preparation of (S)-2-(ethylamino)propan-1-ol (I-2-37)

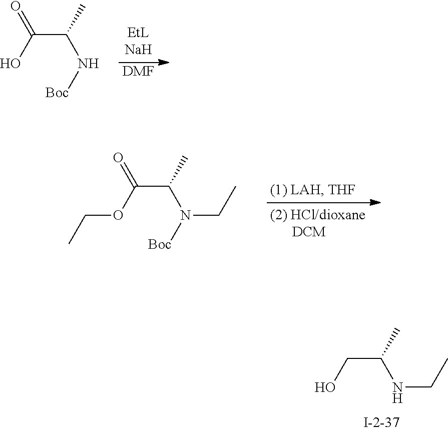

Step 1. To a solution of (2S)-2-(tert-butoxycarbonylamino)propanoic acid (5.00 g, 26.4 mmol, 1 eq) and EtI (37.0 g, 237 mmol, 9 eq) in DMF (50 mL) was added NaH (4.23 g, 105 mmol, 60% purity, 4 eq). The reaction was sealed and stirred at 60° C. On completion, the reaction mixture was quenched by addition of H₂O (150 mL) and extracted with EA (50 mL*3). The combined organic layers were washed with saturated solution of NaCl (50 mL), filtered and concentrated under reduced pressure to give ethyl (2S)-2-[tert-butoxycarbonyl(ethyl)amino]propanoate (6.00 g, 24.4 mmol, 93% yield) as a white solid. ¹H NMR (400 MHz, DMSO-d₆) δ=4.17-3.96 (m, 3H), 3.35-3.05 (m, 2H), 1.43-1.30 (m, 12H), 1.17 (d, J=6.8 Hz, 3H), 1.06 (d, J=5.2 Hz, 3H).

Step 2. To a solution of ethyl (2S)-2-[tert-butoxycarbonyl(ethyl)amino]propanoate (5.00 g, 20.3 mmol, 1 eq) in THE (50 mL) was added LAH (2.5 M, 8.15 mL, 1 eq) at 0° C. The reaction was sealed and stirred at 25° C. On completion, the reaction mixture was quenched with H₂O: NaOH (15%): H₂O (1:1:3), and the residue was obtained by decompression filtration and concentration to give the compound tert-butyl N-ethyl-N-[(1S)-2-hydroxy-1-methyl-ethyl]carbamate (3.50 g, 17.2 mmol, 84% yield) as a white solid. ¹H NMR (400 MHz, CDCl₃) δ=3.90 (s, 1H), 3.53 (d, J=4.4 Hz, 2H), 3.19-2.93 (m, 3H), 1.39 (s, 9H), 1.13-1.02 (m, 6H).

To a solution of the above tert-butyl N-ethyl-N-[(1S)-2-hydroxy-1-methyl-ethyl]carbamate (2.00 g, 9.84 mmol, 1 eq) in DCM (10 mL) was added HCl/dioxane (2 M, 4.92 mL, 1 eq). The reaction was sealed and stirred at 25° C. On completion, the reaction mixture was concentrated under reduced pressure to give the compound (2S)-2-(ethylamino)propan-1-ol (1.00 g, 9.69 mmol, 99% yield) as a white solid (I-2-37).

### Preparation of (S)-2-((2,2-difluoroethyl)amino)propan-1-ol (I-2-38)

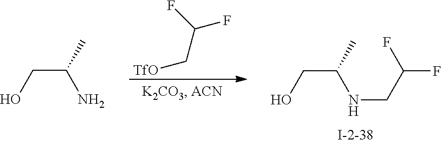

To a solution of (2S)-2-aminopropan-1-ol (2.00 g, 26.6 mmol, 1 eq) in ACN (20 mL) was added K₂CO₃ (3.68 g, 26.6 mmol, 1 eq) and 2,2-difluoroethyl trifluoromethanesulfonate (8.55 g, 39.9 mmol, 1.5 eq). The mixture was stirred at 80° C. for 2 h. On completion, the mixture was filtered to remove K₂CO₃. The residue was purified by column chromatography (SiO₂, Petroleum ether/Ethyl acetate=1:1) to give (2S)-2-(2,2-difluoroethylamino)propan-1-ol (1.30 g, 9.34 mmol, 35% yield) as a colorless oil (I-2-38). ¹H NMR (400 MHz, CDCl₃) δ=5.99-5.64 (m, 1H), 3.55 (dd, J=4.0, 10.8 Hz, 1H), 3.28 (dd, J=7.6, 10.8 Hz, 1H), 3.11-2.74 (m, 3H), 2.18 (br d, J=3.6 Hz, 1H), 1.03 (d, J=6.4 Hz, 3H).

I-2-39 is commercially available.

### Preparation of (S)-2-(((R)-2-((tert-butyldiphenylsilyl)oxy)propyl)amino)propan-1-ol (I-2-40)

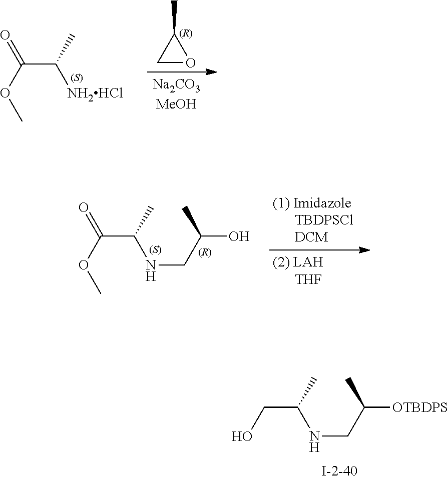

Step 1. To a solution of methyl (2S)-2-aminopropanoate; hydrochloride (2.00 g, 14.3 mmol, 1 eq) in MeOH (20 mL) was added Na₂CO₃ (759 mg, 7.16 mmol, 0.5 eq), and the mixture was stirred at 25° C. for 0.5 h. After filtration, (2R)-2-methyloxirane (2.50 g, 43.0 mmol, 3 eq) was added. The mixture was stirred at 25° C. for 48 h. On completion, the mixture was concentrated to give a residue to give methyl (2S)-2-[[(2R)-2-hydroxypropyl]amino]propanoate (2.31 g, 14.3 mmol, 100% yield) as a colorless oil.

Step 2. To a solution of methyl (2S)-2-[[(2R2-hydroxypropyl]amino]propanoate (2.31 g, 14.3 mmol, 1 eq) in DCM (25 mL) was added Imidazole (2.93 g, 43.0 mmol, 3 eq) and TBDPSCl (4.33 g, 15.8 mmol, 1.1 eq) at 0° C. The mixture was stirred at 25° C. for 12 h. On completion, the mixture was quenched with dilute citric acid (20 mL) and extracted with DCM (25 mL×3), the combined organic phase was dried over anhydrous sodium sulfate, filtered, and concentrated to give a residue. The residue was purified by column chromatography (SiO₂, PE/THF=1:0 to 5:1) to give methyl (2S)-2-[[(2R)-2-[tert-butyl(diphenyl)silyl]oxypropyl]amino]propanoate (722 mg, 1.81 mmol, 13% yield) as a colorless oil. LCMS: (M+1:400.2).

To a solution of the above methyl (2S)-2-[[(2R)-2-[tert-butyl(diphenyl)silyl]oxypropyl]amino]propanoate (670 mg, 1.68 mmol, 1 eq) in THF (7 mL) was added LAH (2.5 M, 2.01 mL, 3 eq) at −40° C. under N₂. The mixture was stirred at −40° C. for 1 h. On completion, the mixture was quenched with sat. NH₄Cl (25 mL) and extracted with ethyl acetate (25 mL×3), the combined organic phase was dried over anhydrous sodium sulfate, filtered, and concentrated to give a residue. The residue was purified by column chromatography (SiO₂, DCM/MeOH=1:0 to 10:1) to give (2S)-2-[[(2R)-2-[tert-butyl(diphenyl) silyl]oxypropyl]amino]propan-1-ol (415 mg, 1.12 mmol, 67% yield) as a brown oil (I-2-40). LCMS: (M+1: 372.2).

### Preparation of (S)-2-((2,2,2-trifluoroethyl)amino)propan-1-ol (I-2-41)

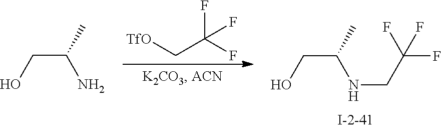

To a solution of (2S)-2-aminopropan-1-ol (2.00 g, 26.6 mmol, 1.00 eq) in ACN (20 mL) was added K₂CO₃ (3.68 g, 26.6 mmol, 1 eq) and 2,2,2-trifluoroethyl trifluoromethanesulfonate (9.27 g, 39.9 mmol, 1.5 eq). The mixture was stirred at 80° C. for 2 h. On completion, the reaction mixture was concentrated under reduced pressure to remove solvent. The residue was purified by column chromatography (SiO₂, Petroleum ether/Ethyl acetate=1/0 to 0/1) to give (2S)-2-(2,2,2-trifluoroethylamino)propan-1-ol (2.20 g, 14.0 mmol, 53% yield) as colorless oil (I-2-41). ¹H NMR (400 MHz, DMSO-d₆) δ=4.63-4.51 (m, 1H), 3.31-3.19 (m, 4H), 2.71-2.61 (m, 1H), 2.23-2.05 (m, 1H), 0.97-0.87 (m, 3H).

### General Method A: Preparation of 5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol (Ex. 1) and Preparation of 5-ethyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol (Ex. 2)

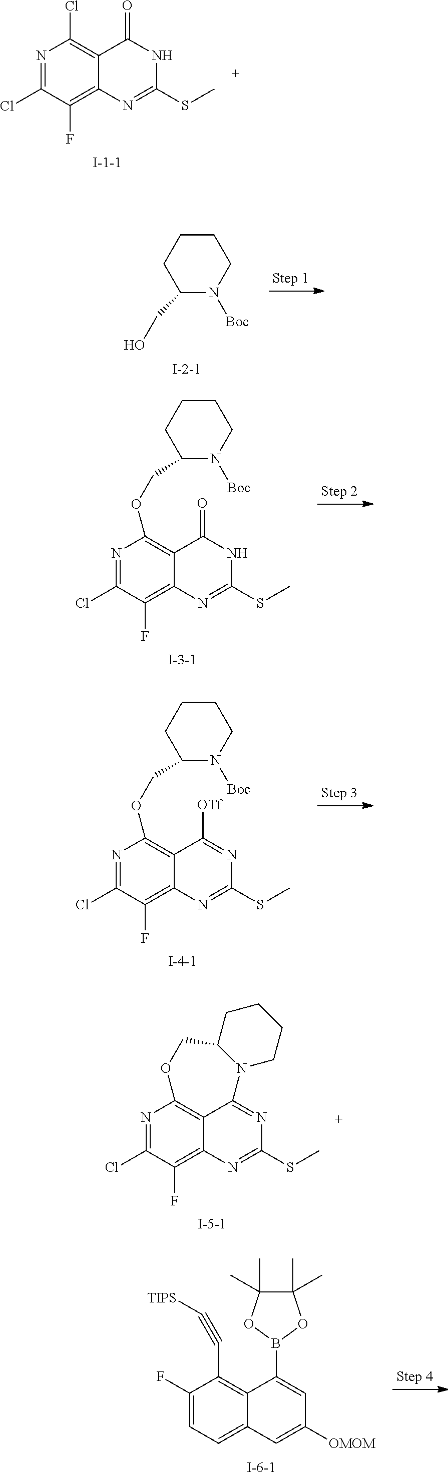

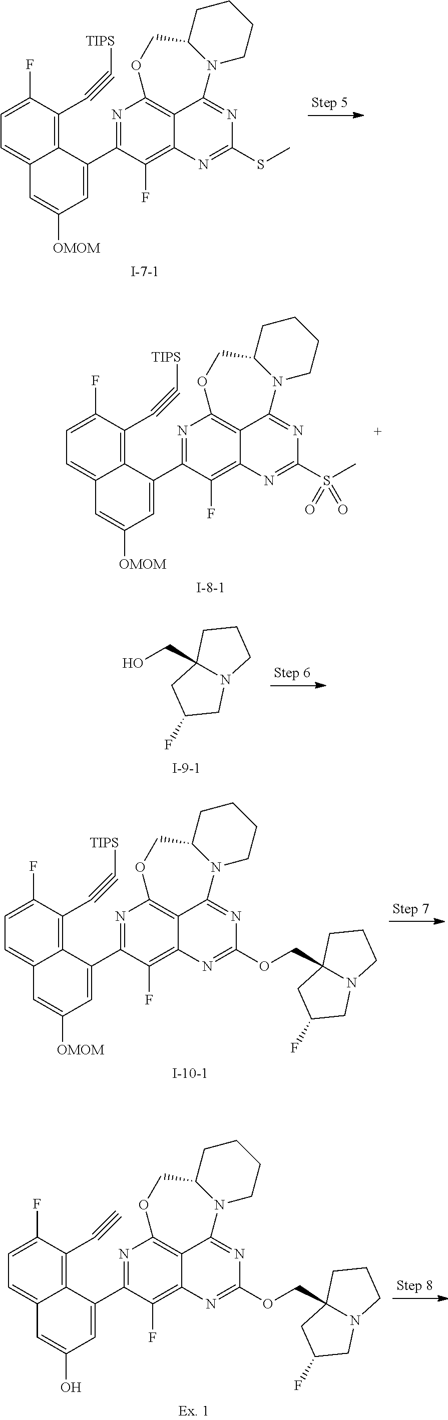

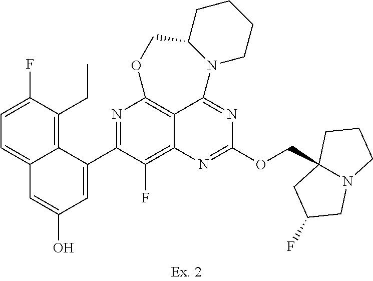 [SMILES: CCc1c(F)ccc2cc(O)cc(-c3nc4c5c(nc(OC[C@@]67CCCN6C[C@H](F)C7)nc5c3F)N3CCCC[C@H]3CO4)c12]

Step 1. To the solution of I-2-1 (1.0 eq.) in DMA at 0° C. is added NaH (1.0 eq.) and the reaction is stirred for 20 minutes before I-1-1 is added. The mixture is stirred to reaction completion. Extraction workup followed by silica gel chromatography affords the product I-3-1.

Step 2. To a solution of I-3-1 (1.0 eq.) and pyridine (1.5 eq.) in DCM at 0° C. is added triflic anhydride (1.3 eq.) dropwise. After reaction completion, water is added to quench reaction. Extraction workup followed by silica gel chromatography affords the product I-4-1.

Step 3. To the solution of I-4-1 (1.0 eq.) in DCM is added TFA (10 eq.) and the mixture is stirred until the Boc deprotection is complete. After removing solvent and TFA under reduced pressure, the residue is dissolved in dioxane and DIEA (3.0 eq.) is added. The mixture is stirred until reaction completion. Extraction workup followed by silica gel chromatography affords the product I-5-1.

Step 4. To a mixture of I-5-1 (1 eq) and I-6-1 (2 eq) in THF is added K₃PO₄ (1.5 M, 1.49 mL, 3 eq) and [2-(2-aminophenyl)phenyl]palladium(II); bis(1-adamantyl)-butyl-phosphane; methanesulfonate (0.1 eq). The resulting mixture is stirred at 60° C. until reaction completion. On completion, the mixture is filtered and concentrated under reduced pressure. The residue is purified by silica gel chromatography to afford product I-7-1.

Step 5. To the solution of I-7-1 (1.0 eq.) in DCM at 0° C. is added mCPBA (2.2 eq.) and mixture is stirred until reaction completion. After quenching with saturated aqueous Na₂S₂O₃, extraction work up with DCM followed by column chromatography affords the product I-8-1.

Step 6. To a mixture of I-8-1 (1.0 eq.) and I-9-1 (2 eq) in dioxane is added DIPEA (3 eq). The resulting mixture is stirred at 80° C. until reaction completion. On completion, extraction workup followed by column chromatography affords product I-10-1.

Step 7. To a solution of I-10-1 (1 eq.) in DCM is added HCl/dioxane (4 M, 40 eq.), and then the mixture is stirred at 25° C. for 1 h. On completion, the mixture is concentrated. The residue is dissolved in DMSO and CsF (6 eq.) is added. The mixture is stirred at 25° C. for 2 h. On completion, the mixture is filtered, and the filtrate is purified by preparative HPLC to afford Ex. 1.

Step 8. The mixture of Ex. 1 and Pd/C in MeOH (1.0 eq.) is degassed three times and hydrogenated under H₂ atmosphere. After the reaction is complete, the mixture is filtered, and the filtrate is concentrated. The residue is purified by HPLC to afford Ex. 2.

### General Method B: Preparation of (8aR,9R)-5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7H-1,3,6,12a-tetraazabenzo[4,5]cyclohepta[1,2,3-de]naphthalen-9-ol (Ex. 4)

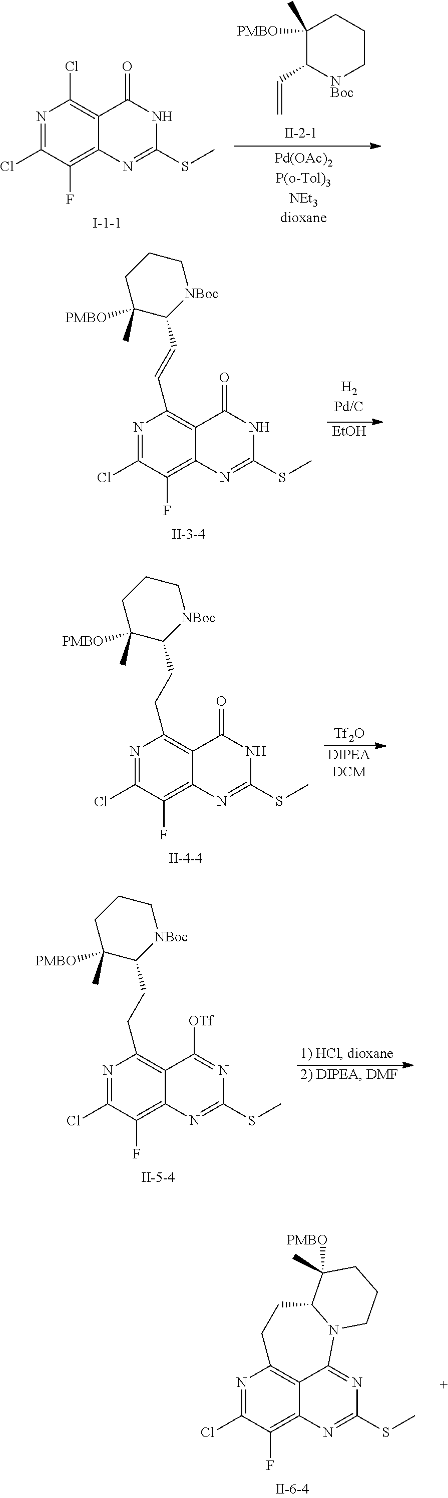

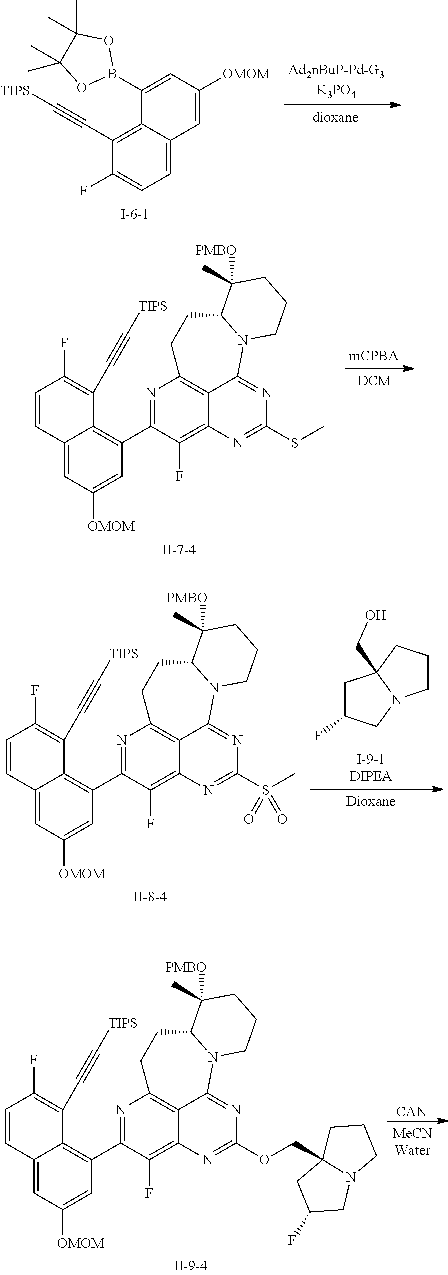

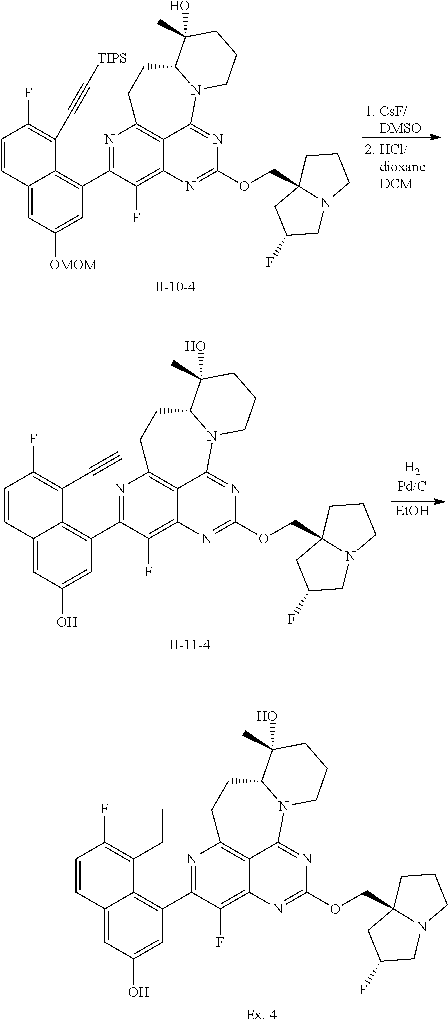

Step 1. To a degassed mixture of I-1-1 (1 eq.), II-2-1 (1 eq.), tri-o-tolylphosphine (0.08 eq.) and Et₃N (3 eq.) in 1,4-dioxane (1.0 M) is added Pd(OAc)₂ (0.02 eq). The mixture is heated at elevated temperature such as 100° C. until reaction complete. The mixture is cooled, diluted with ethyl acetate filtered through celite and concentrated under reduced pressure. Flash chromatography flash chromatography ethyl acetate in hexanes provides II-3-4.

Step 2. To a solution of II-3-4 (1 eq.) in ethanol (0.1 M) is added Pd/C (0.05 eq.) The mixture is placed under an atmosphere of H₂ and stirred until reaction complete. The mixture is filtered through celite and concentrated under reduced pressure. Flash chromatography flash chromatography ethyl acetate in hexanes provides II-4-4.

Step 3. To a solution of II-4-4 (1 eq.) and DIPEA (3 eq.) in dry DCM (0.2 M) at reduced temperature such as −20° C. is added Tf₂O (1.1 eq.). The mixture is slowly warmed to ambient temperature and stirred until reaction complete. The resulting mixture is diluted with water, extracted with EtOAc three times, and the combined extracts are washed with brine, and dried over anhydrous Na₂SO₄. The mixture is concentrated under reduced pressure and purified by flash chromatography ethyl acetate in hexanes to afford II-5-4.

Step 4. To a solution of II-5-4 (1 eq.) in dry DCM (0.2 M) at reduced temperature such as 0° C. is added HCl (1.1 eq., 4 M in dioxane). The mixture is slowly warmed to ambient temperature and stirred until reaction complete. The mixture is concentrated under reduced pressure and resuspended in DMF (0.2 M) and DIPEA added (10 eq.). The mixture is heated to elevated temperature such as 80° C. until the reaction complete. The resulting mixture is diluted with quenched with water, extracted with EtOAc three times, and the combined extracts are washed with brine, and dried over anhydrous Na₂SO₄. The mixture is concentrated under reduced pressure and purified by flash chromatography ethyl acetate in hexanes to afford II-6-4.

Step 5. To a mixture of II-6-4 (1 eq.) and I-6-1 (2 eq.) in THF (0.2 M) is added tripotassium phosphate (3 eq.) and [2-(2-aminophenyl)phenyl]palladium(1+); bis(1-adamantyl)-butyl-phosphane; methanesulfonate (0.1 eq.). The resulting mixture is stirred at elevated temperature such as 80° C. until reaction complete. The mixture is cooled, filtered, and concentrated under reduced pressure. The residue is purified by column chromatography to give II-7-4

Step 6. To a solution of II-7-4 (1 eq.) in dry DCM (0.2 M) at reduced temperature such as 0° C. is added mCPBA (2.1 eq.). The mixture is slowly warmed to ambient temperature and stirred until reaction complete. The organic phase is washed with Na₂SO₃ solution, dried with anhydrous Na₂SO₄, filtered and concentrated under reduced pressure and purified by flash chromatography to afford II-8-4.

Step 7. To a mixture of II-8-4 (1 eq.) and I-9-1 (2 eq.) in dioxane (0.2 M) is added DIPEA (3 eq.). The resulting mixture is stirred at elevated temperature such as 100° C. until reaction complete. The mixture is cooled and concentrated under reduced pressure. The residue is purified by column chromatography to give II-9-4.

Step 8. To a solution of II-9-4 (1 eq.) in MeCN (0.2 M) is added cerium (IV) ammonium nitrate (CAN) (1.0 eq.) dissolved in water. The reaction is stirred at ambient temperature until reaction complete and then DCM and water are added. The layers are separated and the aqueous is extracted with DCM twice more. The combined organic phases are dried, filtered and evaporated and the crude is purified by flash chromatography to afford compound II-10-4.

Step 9: To a solution of II-10-4 (1 eq.) in DMSO (0.2 M) is added CsF (5 eq.) and the mixture is stirred at ambient temperature until reaction complete. On completion, the reaction mixture is partitioned between ethyl acetate and brine and extracted with ethyl acetate. The combined organic layers are dried over Na₂SO₄, filtered and concentrated under reduced pressure. The residue is dissolved in DCM (0.25 M) and HCl/dioxane (4 M, 10 eq.) is added. The resulting mixture is stirred at ambient temperature until reaction complete. On completion, the mixture is concentrated under reduced pressure to provide II-11-4.

Step 10. To a solution of II-11-4 (1 eq.) in ethanol (0.1 M) is added Pd/C (0.05 eq.) The mixture is placed under an atmosphere of H₂ and stirred until reaction complete. The mixture is filtered through celite and concentrated under reduced pressure. The residue is purified by HPLC to afford Ex. 4.

### Preparation of 5-ethynyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-9,10-dimethyl-9,10-dihydro-8H-7-oxa-1,3,6,10-tetraazacyclohepta[1,2,3-de]naphthalen-5-yl]naphthalen-2-ol and 5-ethynyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-9,10-dimethyl-9,10-dihydro-8H-7-oxa-1,3,6,10-tetraazacyclohepta[1,2,3-de]naphthalen-5-yl]naphthalen-2-ol and (Ex. 5a and 5b)

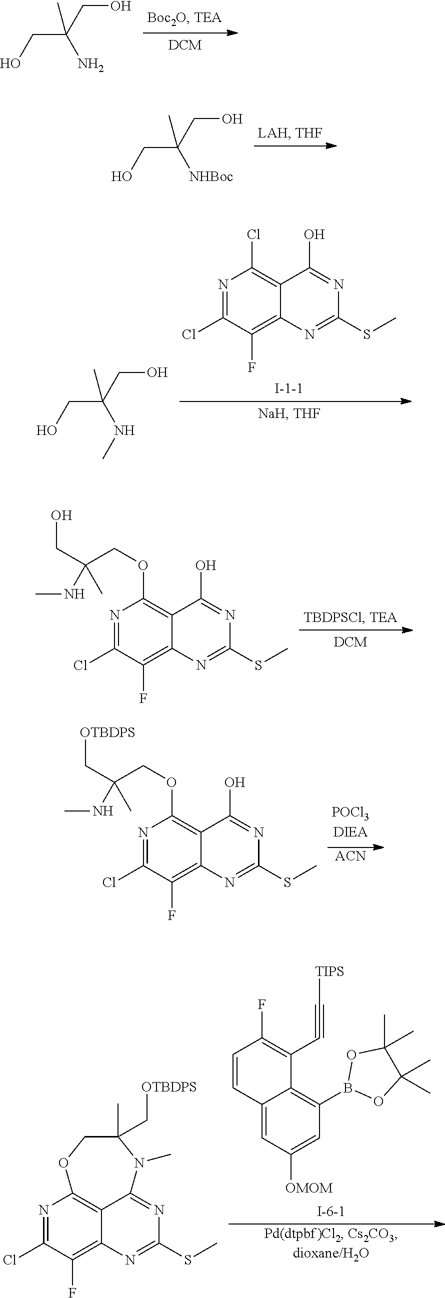

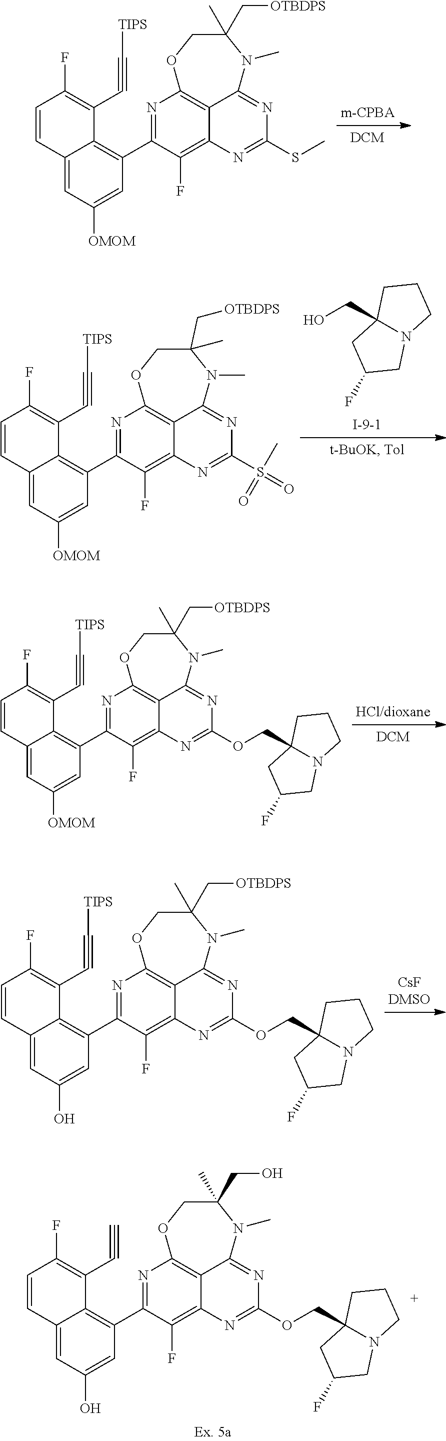

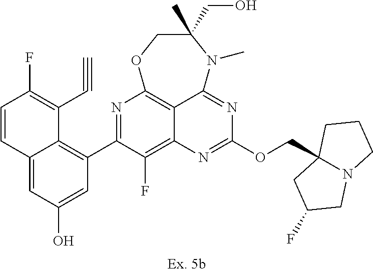 [SMILES: C#Cc1c(F)ccc2cc(O)cc(-c3nc4c5c(nc(OC[C@@]67CCCN6C[C@H](F)C7)nc5c3F)N(C)[C@@](C)(CO)CO4)c12]

Step 1. To a solution of 2-amino-2-methyl-propane-1,3-diol (2.00 g, 19.0 mmol, 1 eq) in DCM (20 mL) was added TEA (1.92 g, 19.0 mmol, 2.65 mL, 1 eq) and tert-butoxycarbonyl tert-butyl carbonate (4.98 g, 22.8 mmol, 5.24 mL, 1.2 eq). The mixture was stirred at 25° C. for 16 hr. On completion, the mixture was concentrated in vacuum to give a residue. The residue was purified by column chromatography (SiO₂, Petroleum ether/Ethyl acetate=1/0 to 0/1) to give tert-butyl N-[2-hydroxy-1-(hydroxymethyl)-1-methyl-ethyl]carbamate (3.13 g, 15.3 mmol, 80% yield) as a white solid. ¹H NMR (400 MHz, DMSO-d₆) δ=5.87 (s, 1H), 4.58 (t, J=6.0 Hz, 2H), 3.45-3.39 (m, 2H), 3.38-3.33 (m, 2H), 1.36 (s, 9H), 1.10 (s, 3H).

Step 2. To a solution of tert-butyl N-[2-hydroxy-1-(hydroxymethyl)-1-methyl-ethyl]carbamate (3.00 g, 14.6 mmol, 1 eq) in THF (30 mL) was added LAH (2.5 M, 17.5 mL, 3 eq) at 0° C. The mixture was stirred at 60° C. for 3 hr. On completion, the mixture was quenched with water (1 mL), sat. 15% NaOH (1 mL), followed by water (3 mL), filtered, and filtrate concentrated in vacuum to give 2-methyl-2-(methylamino)propane-1,3-diol (2.20 g, crude) as a colorless oil. ¹H NMR (400 MHz, DMSO-d6) δ=3.19 (d, J=3.6 Hz, 4H), 2.14 (s, 3H), 0.81 (s, 3H).

Step 3. To a solution of 2-methyl-2-(methylamino)propane-1,3-diol (800 mg, 6.71 mmol, 4 eq) in THF (10 mL) was added Sodium hydride (336 mg, 8.39 mmol, 60% purity, 5 eq) at 0° C., and the mixture was stirred at 0° C. for 0.5 hr. 5,7-dichloro-8-fluoro-2-methylsulfanyl-3H-pyrido[4,3-d]pyrimidin-4-one (470 mg, 1.68 mmol, 1 eq, I-1-1) was then added, and the mixture was stirred at 25° C. for 16 hr. On completion, the reaction mixture diluted with MeOH (10 mL) and concentrated in vacuum to give a residue. The residue was purified by column chromatography (SiO₂, DCM/MeOH=1/0 to 4/1) to give 7-chloro-8-fluoro-5-[3-hydroxy-2-methyl-2-(methylamino)propoxy]-2-methylsulfanyl-pyrido[4,3-d]pyrimidin-4-ol (267 mg, 0.655 mmol, 39% yield, 89% purity) as white solid. LCMS: (M+1: 363.0).

Step 4. To a solution of 7-chloro-8-fluoro-5-[3-hydroxy-2-methyl-2-(methylamino)propoxy]-2-methylsulfanyl-pyrido[4,3-d]pyrimidin-4-ol (260 mg, 0.717 mmol, 1 eq) in THF (5 mL) was added TEA (218 mg, 2.15 mmol, 0.3 mL, 3 eq), TBDPSCl (394 mg, 1.43 mmol, 0.37 mL, 2 eq) and DMAP (8.75 mg, 0.072 mmol, 0.1 eq). The mixture was stirred at 50° C. for 16 hr. On completion, the mixture was quenched with water (20 mL) and extracted with ethyl acetate (10 mL×3), the combined organic phase was washed with brine (10 mL×3), dried over anhydrous sodium sulfate, filtered and concentrated in vacuum to give a residue. The residue was purified by column chromatography (SiO₂, DCM/MeOH=1/0 to 10/1) to give 5-[3-[tert-butyl(diphenyl)silyl]oxy-2-methyl-2-(methylamino)propoxy]-7-chloro-8-fluoro-2-methylsulfanyl-pyrido [4,3-d]pyrimidin-4-ol (330 mg, 0.483 mmol, 67% yield, 88% purity) as a yellow solid. LCMS: (M+1: 601.1).

Step 5. To a solution of 5-[3-[tert-butyl(diphenyl)silyl]oxy-2-methyl-2-(methylamino)propoxy]-7-chloro-8-fluoro-2-methylsulfanyl-pyrido [4,3-d]pyrimidin-4-ol (300 mg, 0.499 mmol, 1 eq) in ACN (3 mL) was added POCl₃ (230 mg, 1.50 mmol, 0.140 mL, 3 eq) and DIEA (322 mg, 2.49 mmol, 0.435 mL, 5 eq). The mixture was stirred at 80° C. for 2 hr. On completion, the mixture was quenched with sat. NaHCO₃ (20 mL) and extracted with ethyl acetate (10 mL×3). The combined organic phase was dried over anhydrous sodium sulfate, filtered, and concentrated in vacuum to give a residue. The residue was purified by column chromatography (SiO₂, PE/EA=1/0 to 2/1) to give tert-butyl-[(7-chloro-6-fluoro-12,13-dimethyl-3-methylsulfanyl-10-oxa-2,4,8,13-tetrazatricyclo[7.4.1.0^{5,14}]tetradeca-1,3,5,7,9 (14)-pentaen-12-yl)methoxy]-diphenyl-silane (108 mg, 0.167 mmol, 33% yield, 90% purity) as a colorless oil. LCMS: (M+1:583.1).

Step 6. A mixture of tert-butyl-[(7-chloro-6-fluoro-12,13-dimethyl-3-methylsulfanyl-10-oxa-2,4,8,13-tetrazatricyclo[7.4.1.0^{5,14}]tetradeca-1,3,5,7,9 (14)-pentaen-12-yl)methoxy]-diphenyl-silane (100 mg, 0.171 mmol, 1 eq), 2-[2-fluoro-6-(methoxymethoxy)-8-(4,4,5,5-tetramethyl-1,3,2-dioxaborolan-2-yl)-1-naphthyl]ethynyl-triisopropyl-silane (105 mg, 0.206 mmol, 1.2 eq, I-6-1), Pd(dtbpf)Cl₂ (12.0 mg, 0.017 mmol, 0.1 eq), and Cs₂CO₃ (168 mg, 0.514 mmol, 3 eq) in dioxane (5 mL) and H₂O (1 mL) was degassed and purged with N₂ 3 times, and the mixture was stirred at 80° C. for 2 hr under N₂ atmosphere. On completion, the mixture was filtered, and the filtrate was concentrated to give a residue. The residue was purified by column chromatography (SiO₂, Petroleum ether/Ethyl acetate=1/0 to 0/1) to give tert-butyl-[[6-fluoro-7-[7-fluoro-3-(methoxymethoxy)-8-(2-triisopropylsilylethynyl)-1-naphthyl]-12,13-dimethyl-3-methylsulfanyl-10-oxa-2,4,8,13-tetrazatricyclo[7.4.1.0^{5,14}]tetradeca-1,3,5,7,9 (14)-pentaen-12-yl]methoxy]-diphenyl-silane (150 mg, 0.096 mmol, 56% yield, 60% purity) as a colorless oil. LCMS: (M+1:933.2).

Step 7. To a solution of tert-butyl-[[6-fluoro-7-[7-fluoro-3-(methoxymethoxy)-8-(2-triisopropylsilylethynyl)-1-naphthyl]-12,13-dimethyl-3-methylsulfanyl-10-oxa-2,4,8,13-tetrazatricyclo[7.4.1.05,14]tetradeca-1,3,5,7,9 (14)-pentaen-12-yl]methoxy]-diphenyl-silane (120 mg, 0.077 mmol, 1 eq) in DCM (2 mL) was added m-CPBA (47.0 mg, 0.231 mmol, 85% purity, 3 eq) at 0° C., and the mixture was stirred at 25° C. for 1 hr. On completion, the reaction mixture diluted with sat. Na₂SO₃ (10 mL) and extracted with DCM (5 mL×3), the combined organic phase was dried over anhydrous sodium sulfate, filtered, and concentrated under reduced pressure to give tert-butyl-[[6-fluoro-7-[7-fluoro-3-(methoxymethoxy)-8-(2-triisopropylsilylethynyl)-1-naphthyl]-12,13-dimethyl-3-methylsulfonyl-10-oxa-2,4,8,13-tetrazatricyclo[7.4.1.05,14]tetradeca-1,3,5,7,9 (14)-pentaen-12-yl]methoxy]-diphenyl-silane (60.0 mg, crude) as a colorless oil. LCMS: (M+1: 965.4).

Step 8. A mixture of [(2R,8S)-2-fluoro-1,2,3,5,6,7-hexahydropyrrolizin-8-yl]methanol (30.0 mg, 0.186 mmol, 3 eq, I-9-1) and t-BuOK (21.0 mg, 0.186 mmol, 3 eq) in toluene (0.5 mL) was degassed and purged with N₂ 3 times, and stirred at 0° C. for 0.5 h under N₂ atmosphere. A solution of tert-butyl-[[6-fluoro-7-[7-fluoro-3-(methoxymethoxy)-8-(2-triisopropylsilylethynyl)-1-naphthyl]-12,13-dimethyl-3-methylsulfonyl-10-oxa-2,4,8,13-tetrazatricyclo[7.4.1.05,14]tetradeca-1,3,5,7,9 (14)-pentaen-12-yl]methoxy]-diphenyl-silane (60.0 mg, 0.062 mmol, 1 eq) in toluene (0.5 mL) was then added to the above mixture at 0° C., and then the mixture was stirred at 25° C. for 1 hr under N₂ atmosphere. On completion, the mixture was quenched with water (10 mL) and extracted with ethyl acetate (5 mL×3), the combined organic phase was washed with brine (5 mL×3), dried over anhydrous sodium sulfate, filtered, and concentrated in vacuum to give a residue. The residue was purified by column chromatography (SiO₂, Petroleum ether/THF=1/0 to 0/1) to give tert-butyl-[[6-fluoro-3-[[(2R,8S)-2-fluoro-1,2,3,5,6,7-hexahydropyrrolizin-8-yl]methoxy]-7-[7-fluoro-3-(methoxymethoxy)-8-(2-triisopropylsilylethynyl)-1-naphthyl]-12,13-dimethyl-10-oxa-2,4,8,13-tetrazatricyclo[7.4.1.05,14]tetradeca-1,3,5,7,9 (14)-pentaen-12-yl]methoxy]-diphenyl-silane (23.0 mg, crude) as a colorless oil. LCMS: (M+1: 1044.5).

Step 9. To a solution of tert-butyl-[[6-fluoro-3-[[(2R,8S)-2-fluoro-1,2,3,5,6,7-hexahydropyrrolizin-8-yl]methoxy]-7-[7-fluoro-3-(methoxymethoxy)-8-(2-triisopropylsilylethynyl)-1-naphthyl]-12,13-dimethyl-10-oxa-2,4,8,13-tetrazatricyclo[7.4.1.0^{5,14}]tetradeca-1,3,5,7,9 (14)-pentaen-12-yl]methoxy]-diphenyl-silane (20.0 mg, 0.019 mmol, 1 eq) in DCM (1 mL) was added HCl/dioxane (2 M, 0.2 mL, 20 eq). The mixture was stirred at 25° C. for 1 hr. On completion, the mixture was filtered and concentrated to give 4-[12-[[tert-butyl(diphenyl)silyl]oxymethyl]-6-fluoro-3-[[(2R,8S)-2-fluoro-1,2,3,5,6,7-hexahydropyrrolizin-8-yl]methoxy]-12,13-dimethyl-10-oxa-2,4,8,13-tetrazatricyclo[7.4.1.0^{5,14}]tetradeca-1,3,5,7,9 (14)-pentaen-7-yl]-6-fluoro-5-(2-triisopropylsilylethynyl)naphthalen-2-ol (20.0 mg, crude) as a red oil. LCMS: (M+1: 1000.5).

Step 10. To a solution of 4-[12-[[tert-butyl(diphenyl)silyl]oxymethyl]-6-fluoro-3-[[(2R,8S)-2-fluoro-1,2,3,5,6,7-hexahydropyrrolizin-8-yl]methoxy]-12,13-dimethyl-10-oxa-2,4,8,13-tetrazatricyclo[7.4.1.05,14]tetradeca-1,3,5,7,9 (14)-pentaen-7-yl]-6-fluoro-5-(2-triisopropylsilylethynyl)naphthalen-2-ol (20.0 mg, 0.020 mmol, 1 eq) in DMSO (1 mL) was added Cesium fluoride (15.0 mg, 0.100 mmol, 0.004 mL, 5 eq). The mixture was stirred at 25° C. for 5 hr. On completion, the mixture was filtered and concentrated to give a residue. The residue was purified by prep-HPLC to give a mixture of 5-ethynyl-6-fluoro-4-((R)-4-fluoro-2-(((2R,7aS)-2-fluorotetrahydro-1H-pyrrolizin-7a(5H)-yl)methoxy)-9-(hydroxymethyl)-9,10-dimethyl-9,10-dihydro-8H-7-oxa-1,3,6,10-tetraazacyclohepta[de]naphthalen-5-yl)naphthalen-2-ol and 5-ethynyl-6-fluoro-4-((S)-4-fluoro-2-(((2R,7aS)-2-fluorotetrahydro-1H-pyrrolizin-7a(5H)-yl)methoxy)-9-(hydroxymethyl)-9,10-dimethyl-9,10-dihydro-8H-7-oxa-1,3,6,10-tetraazacyclohepta[de]naphthalen-5-yl)naphthalen-2-ol (0.29 mg) as a red solid (Ex. 5a and 5b). ¹H NMR (400 MHz, MeOD-d₄) δ=7.85 (dd, J=5.6, 9.2 Hz, 1H), 7.36-7.28 (m, 2H), 7.25-7.18 (m, 1H), 5.65-5.44 (m, 1H), 4.71 (d, J=8.4 Hz, 1H), 4.67-4.63 (m, 2H), 4.53 (d, J=4.0 Hz, 1H), 4.07-3.91 (m, 2H), 3.86 (dd, J=2.4, 15.2 Hz, 2H), 3.77 (dd, J=7.6, 12.0 Hz, 1H), 3.50-3.44 (m, 2H), 3.41 (s, 3H), 2.75-2.53 (m, 2H), 2.47-2.27 (m, 3H), 2.20-2.03 (m, 1H), 1.49 (d, J=10.0 Hz, 3H); LCMS: (M+1: 606.2).

The diastereomers can be resolved by supercritical fluid chromatography (SFC) to provide the individual diastereomers Ex. 5a and Ex. 5b using known procedures and instruments.

### General Method C: Preparation of 5-ethynyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-9,10-dimethyl-7,8,9,10-tetrahydro-1,3,6,10-tetraazacyclohepta[1,2,3-de]naphthalen-5-yl]naphthalen-2-ol (Ex. 7) and 5-ethyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-9,10-dimethyl-7,8,9,10-tetrahydro-1,3,6,10-tetraazacyclohepta[1,2,3-de]naphthalen-5-yl]naphthalen-2-ol

### (Ex. 8):

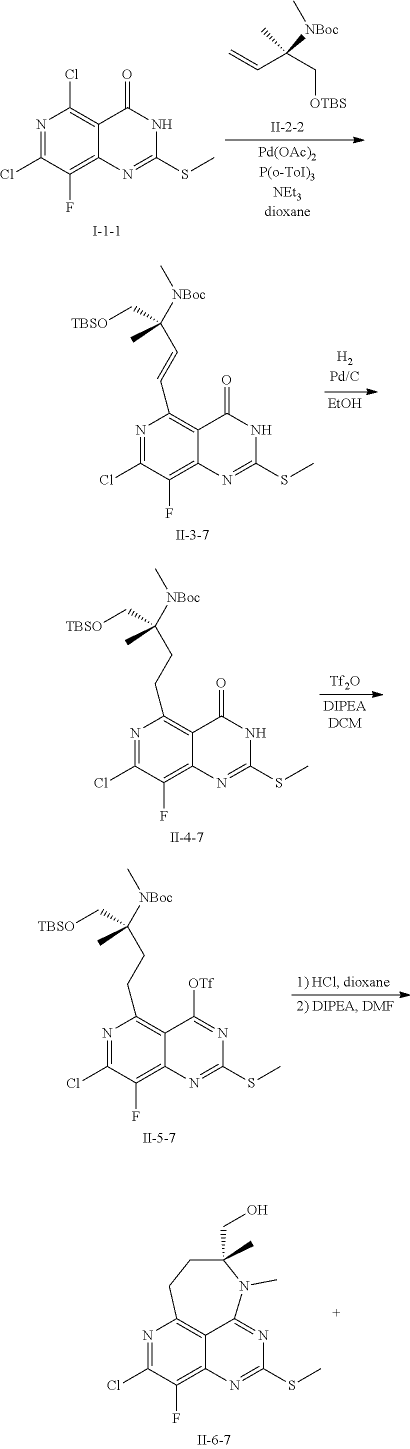

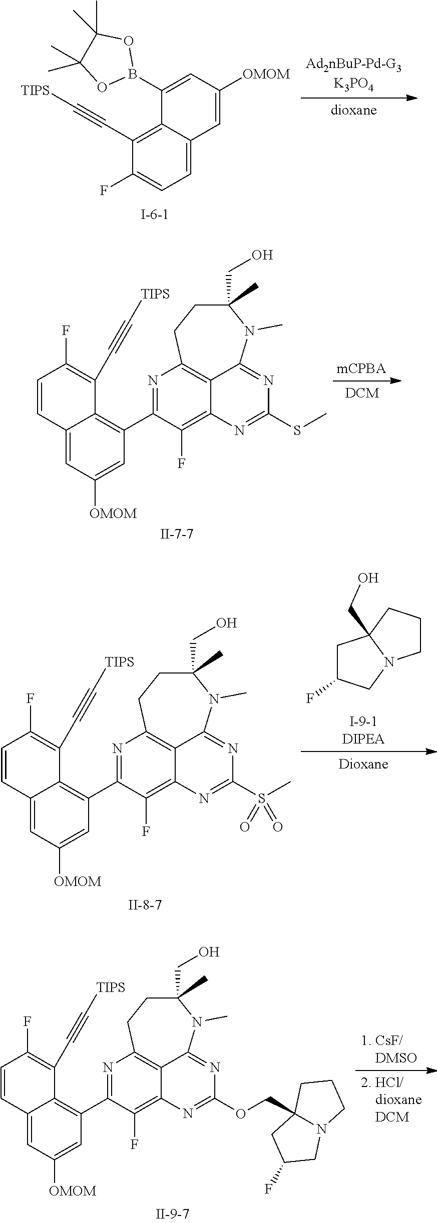

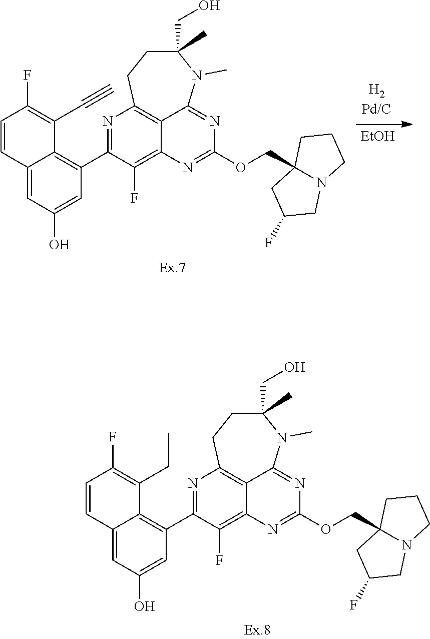 [SMILES: "EtOH" ; C#Cc1c(F)ccc2cc(O)cc(-c3nc4c5c(nc(OC[C@@]67CCCN6C[C@H](F)C7)nc5c3F)N(C)[C@@](C)(CO)CC4)c12 ; CCc1c(F)ccc2cc(O)cc(-c3nc4c5c(nc(OC[C@@]67CCCN6C[C@H](F)C7)nc5c3F)N(C)[C@@](C)(CO)CC4)c12]

Steps 1 through 7 are performed in a manner similar to steps 1 through 7 in General Method B starting with I-1-1 and II-2-2 to provide II-9-7.

Step 8 was performed in a manner similar to step 9 of General Method B starting with II-9-7. The residue of this reaction is purified by HPLC to afford Ex. 7.

Step 9 was performed in a manner similar to step 10 of General Method B starting with Ex. 7 to give Ex. 8.

Ex. 1-Ex. 22 can be prepared, for example, using the corresponding methods and intermediates as shown in the table below.

| General | | Ex. | |
|---|---|---|---|
| Method | Intermediate | # | Structure |
| A | 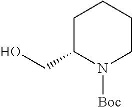 [SMILES: CC(C)(C)OC(=O)N1CCCC[C@H]1CO] | 1 | 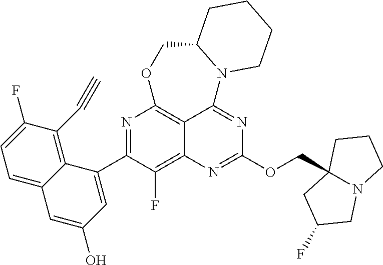 [SMILES: C#Cc1c(F)ccc2cc(O)cc(-c3nc4c5c(nc(OC[C@@]67CCCN6C[C@H](F)C7)nc5c3F)N3CCCC[C@H]3CO4)c12] |
| A | 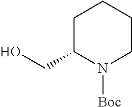 [SMILES: CC(C)(C)OC(=O)N1CCCC[C@H]1CO] | 2 | 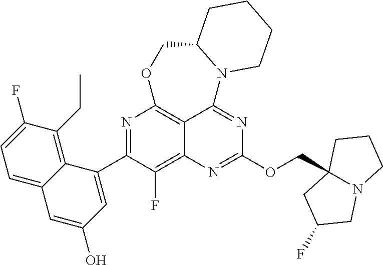 [SMILES: CCc1c(F)ccc2cc(O)cc(-c3nc4c5c(nc(OC[C@@]67CCCN6C[C@H](F)C7)nc5c3F)N3CCCC[C@H]3CO4)c12] |
| A | 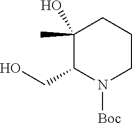 [SMILES: CC(C)(C)OC(=O)N1CCC[C@@](C)(O)[C@H]1CO] | 3 | 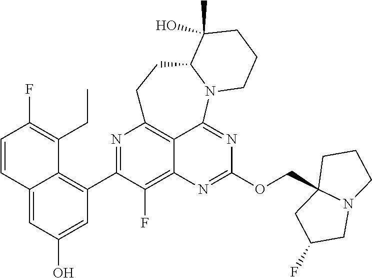 [SMILES: CCc1c(F)ccc2cc(O)cc(-c3nc4c5c(nc(OC[C@@]67CCCN6C[C@H](F)C7)nc5c3F)N3CCC[C@@](C)(O)[C@H]3CC4)c12] |
| B | 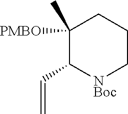 | 4 | 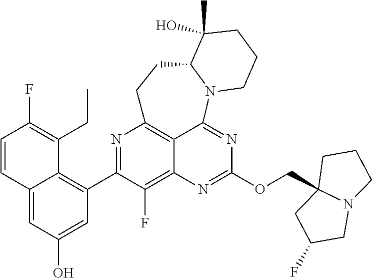 [SMILES: CCc1c(F)ccc2cc(O)cc(-c3nc4c5c(nc(OC[C@@]67CCCN6C[C@H](F)C7)nc5c3F)N3CCC[C@@](C)(O)[C@H]3CC4)c12] |
| A | 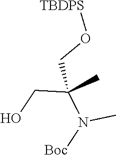 | 5 | 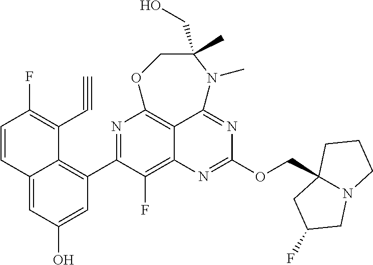 [SMILES: C#Cc1c(F)ccc2cc(O)cc(-c3nc4c5c(nc(OC[C@@]67CCCN6C[C@H](F)C7)nc5c3F)N(C)[C@@](C)(CO)CO4)c12] |
| A | 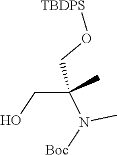 | 6 | 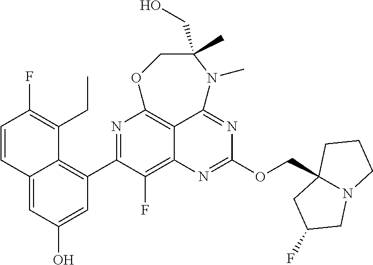 [SMILES: CCc1c(F)ccc2cc(O)cc(-c3nc4c5c(nc(OC[C@@]67CCCN6C[C@H](F)C7)nc5c3F)N(C)[C@@](C)(CO)CO4)c12] |
| C | 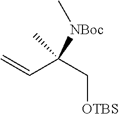 [SMILES: C=C[C@](C)(CO[Si](C)(C)C(C)(C)C)N(C)C(=O)OC(C)(C)C] | 7 | 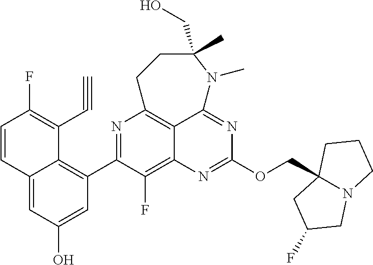 [SMILES: C#Cc1c(F)ccc2cc(O)cc(-c3nc4c5c(nc(OC[C@@]67CCCN6C[C@H](F)C7)nc5c3F)N(C)[C@@](C)(CO)CC4)c12] |
| C | 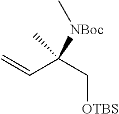 [SMILES: C=C[C@](C)(CO[Si](C)(C)C(C)(C)C)N(C)C(=O)OC(C)(C)C] | 8 | 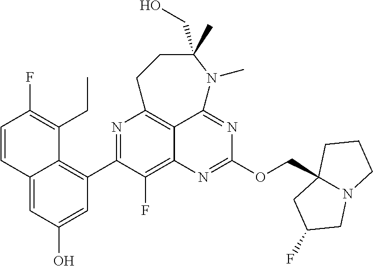 [SMILES: CCc1c(F)ccc2cc(O)cc(-c3nc4c5c(nc(OC[C@@]67CCCN6C[C@H](F)C7)nc5c3F)N(C)[C@@](C)(CO)CC4)c12] |
| C | 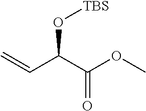 | 9 | 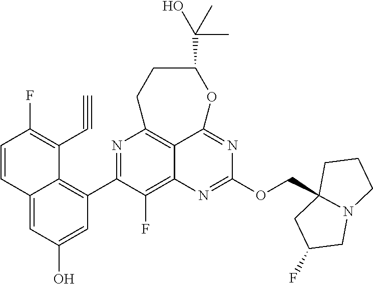 [SMILES: C#Cc1c(F)ccc2cc(O)cc(-c3nc4c5c(nc(OC[C@@]67CCCN6C[C@H](F)C7)nc5c3F)O[C@@H](C(C)(C)O)CC4)c12] |
| C | 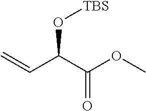 | 10 | 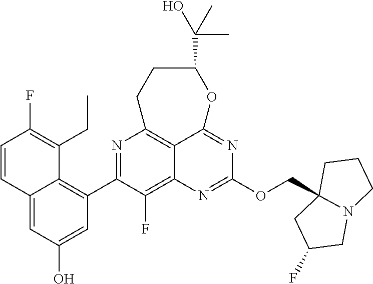 [SMILES: CCc1c(F)ccc2cc(O)cc(-c3nc4c5c(nc(OC[C@@]67CCCN6C[C@H](F)C7)nc5c3F)O[C@@H](C(C)(C)O)CC4)c12] |
| C | 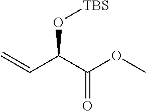 | 11 | 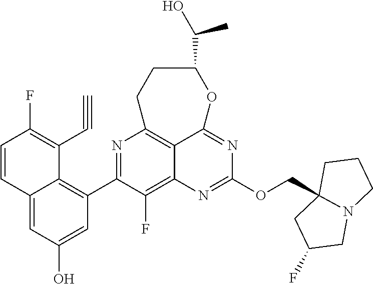 [SMILES: C#Cc1c(F)ccc2cc(O)cc(-c3nc4c5c(nc(OC[C@@]67CCCN6C[C@H](F)C7)nc5c3F)O[C@@H]([C@H](C)O)CC4)c12] |
| C | 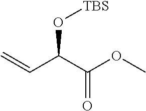 | 12 | 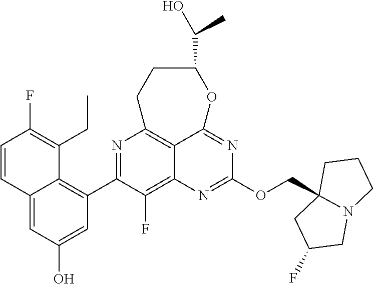 [SMILES: CCc1c(F)ccc2cc(O)cc(-c3nc4c5c(nc(OC[C@@]67CCCN6C[C@H](F)C7)nc5c3F)O[C@@H]([C@H](C)O)CC4)c12] |
| C | 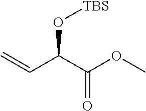 | 13 | 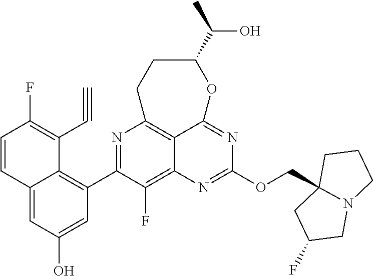 [SMILES: C#Cc1c(F)ccc2cc(O)cc(-c3nc4c5c(nc(OC[C@@]67CCCN6C[C@H](F)C7)nc5c3F)O[C@@H]([C@@H](C)O)CC4)c12] |
| C | 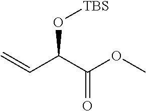 | 14 | 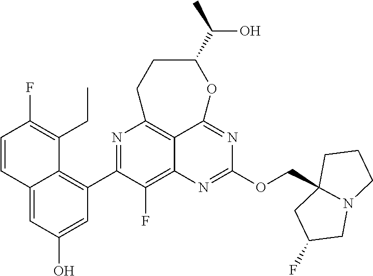 [SMILES: CCc1c(F)ccc2cc(O)cc(-c3nc4c5c(nc(OC[C@@]67CCCN6C[C@H](F)C7)nc5c3F)O[C@@H]([C@@H](C)O)CC4)c12] |
| C | 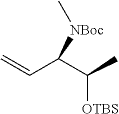 [SMILES: C=C[C@H]([C@@H](C)O[Si](C)(C)C(C)(C)C)N(C)C(=O)OC(C)(C)C] | 15 | 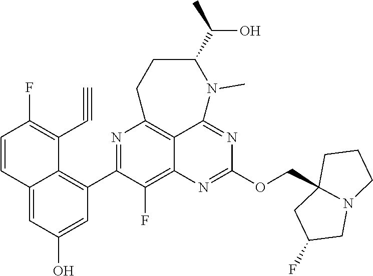 [SMILES: C#Cc1c(F)ccc2cc(O)cc(-c3nc4c5c(nc(OC[C@@]67CCCN6C[C@H](F)C7)nc5c3F)N(C)[C@@H]([C@@H](C)O)CC4)c12] |
| C | 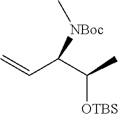 [SMILES: C=C[C@H]([C@@H](C)O[Si](C)(C)C(C)(C)C)N(C)C(=O)OC(C)(C)C] | 16 | 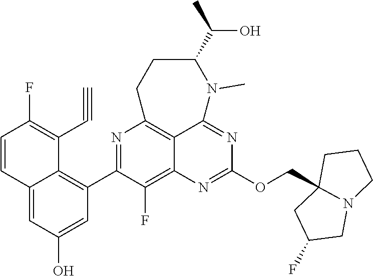 [SMILES: C#Cc1c(F)ccc2cc(O)cc(-c3nc4c5c(nc(OC[C@@]67CCCN6C[C@H](F)C7)nc5c3F)N(C)[C@@H]([C@@H](C)O)CC4)c12] |
| C | 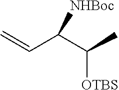 [SMILES: C=C[C@@H](NC(=O)OC(C)(C)C)[C@@H](C)O[Si](C)(C)C(C)(C)C] | 17 | 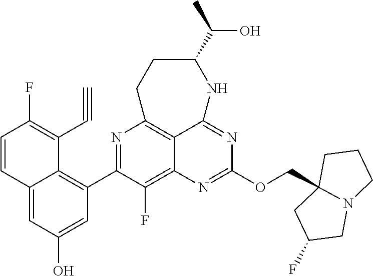 [SMILES: C#Cc1c(F)ccc2cc(O)cc(-c3nc4c5c(nc(OC[C@@]67CCCN6C[C@H](F)C7)nc5c3F)N[C@@H]([C@@H](C)O)CC4)c12] |
| C | 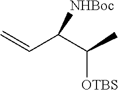 [SMILES: C=C[C@@H](NC(=O)OC(C)(C)C)[C@@H](C)O[Si](C)(C)C(C)(C)C] | 18 | 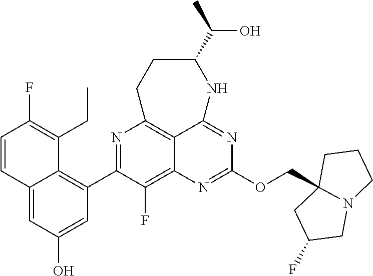 [SMILES: CCc1c(F)ccc2cc(O)cc(-c3nc4c5c(nc(OC[C@@]67CCCN6C[C@H](F)C7)nc5c3F)N[C@@H]([C@@H](C)O)CC4)c12] |
| A | 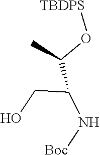 | 19 | 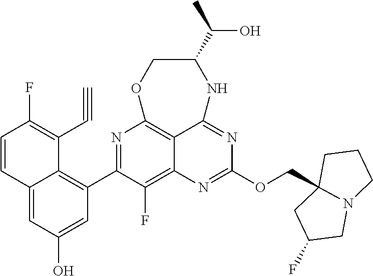 [SMILES: C#Cc1c(F)ccc2cc(O)cc(-c3nc4c5c(nc(OC[C@@]67CCCN6C[C@H](F)C7)nc5c3F)N[C@@H]([C@@H](C)O)CO4)c12] |
| A | 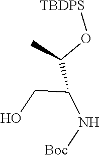 | 20 | 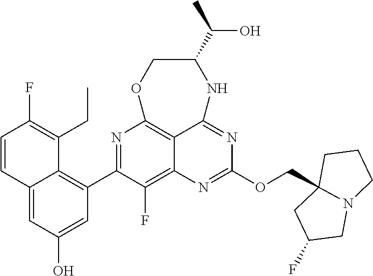 [SMILES: CCc1c(F)ccc2cc(O)cc(-c3nc4c5c(nc(OC[C@@]67CCCN6C[C@H](F)C7)nc5c3F)N[C@@H]([C@@H](C)O)CO4)c12] |
| C | 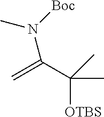 [SMILES: C=C(N(C)C(=O)OC(C)(C)C)C(C)(C)O[Si](C)(C)C(C)(C)C] | 21 | 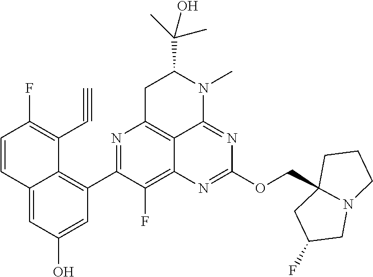 [SMILES: C#Cc1c(F)ccc2cc(O)cc(-c3nc4c5c(nc(OC[C@@]67CCCN6C[C@H](F)C7)nc5c3F)N(C)[C@@H](C(C)(C)O)C4)c12] |
| C | 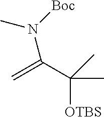 [SMILES: C=C(N(C)C(=O)OC(C)(C)C)C(C)(C)O[Si](C)(C)C(C)(C)C] | 22 | 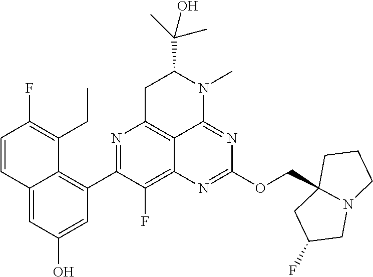 [SMILES: CCc1c(F)ccc2cc(O)cc(-c3nc4c5c(nc(OC[C@@]67CCCN6C[C@H](F)C7)nc5c3F)N(C)[C@@H](C(C)(C)O)C4)c12] |

Ex. 23 and Ex. 24 are prepared with general method C as Ex. 18 and using the corresponding boronic esters.

### Preparation of 5-ethynyl-6-fluoro-4-((S)-4-fluoro-2-(((2R,7aS)-2-fluorotetrahydro-1H-pyrrolizin-7a(5H)-yl)methoxy)-8,8a,9,10,11,12-hexahydro-7-oxa-1,3,6,12a-tetraazabenzo[4,5]cyclohepta[1,2,3-de]naphthalen-5-yl)naphthalen-2-ol (Ex. 1)

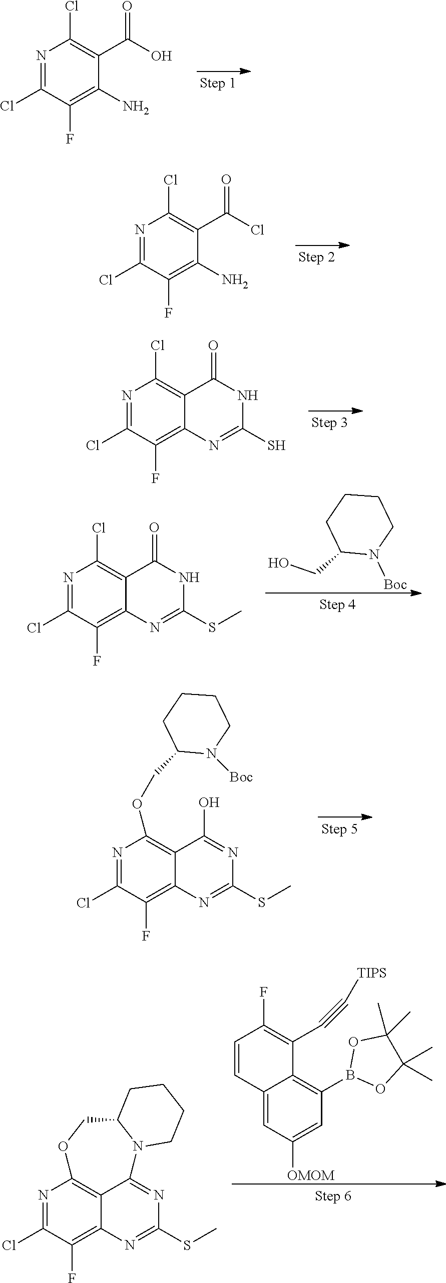

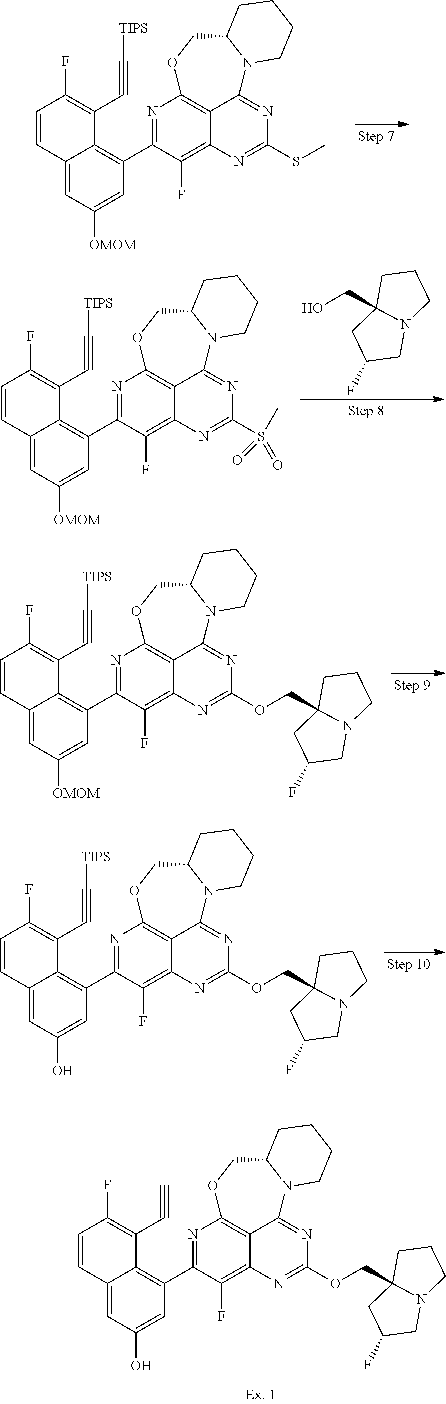

Step 1. 4-amino-2,6-dichloro-5-fluoropyridine-3-carboxylic acid (2.9 g, 12.89 mmol, 1 eq.) was added to SOCl₂ (82.00 g, 689.25 mmol, 53.48 eq) and the mixture was stirred at 50° C. for 1 h. On completion, the reaction mixture was concentrated under reduced pressure to remove SOCl₂ to give 4-amino-2,6-dichloro-5-fluoropyridine-3-carbonyl chloride (2.2 g, 9.04 mmol, crude) as brown oil. Step 2. To a solution of 4-amino-2,6-dichloro-5-fluoropyridine-3-carbonyl chloride (2.1 g, 8.63 mmol, 1 eq) in ACETONE (60 mL) was added NH₄SCN (1.97 g, 25.88 mmol, 3 eq). The mixture was stirred at 25° C. for 1 h. On completion, the mixture was concentrated to remove solvent to give 5,7-dichloro-8-fluoro-2-sulfanylpyrido[4,3-d]pyrimidin-4 (3H)-one (1.2 g, 4.51 mmol, crude) as brown solid. LCMS: m/z 263.1 (M+1).

Step 3. To a solution of 5,7-dichloro-8-fluoro-2-sulfanylpyrido[4,3-d]pyrimidin-4 (3H)-one (1.1 g, 4.13 mmol, 1 eq) in MeOH (7.5 mL) was added NaOH (0.1 M, 82.68 mL, 2 eq) and Mel (1.17 g, 8.27 mmol, 2 eq). The mixture was stirred at 25° C. for 1 hr. On completion, the mixture was diluted with water (50 mL), added 6M HCl to adjust pH to 6, filtered and the filtrate was concentrated under reduced pressure to remove MeOH to give 5,7-dichloro-8-fluoro-2-(methylsulfanyl)pyrido[4,3-d]pyrimidin-4 (3H)-one (1.1 g, 3.93 mmol, crude) as gray solid. LCMS: m/z 279.9 (M+1)

Step 4. To a solution of tert-butyl (2S)-2-(hydroxymethyl)piperidine-1-carboxylate (845.46 mg, 3.93 mmol, 1 eq) in THE (10 mL) was added NaH (314.14 mg, 7.85 mmol, 60% purity, 2 eq) at 0° C. and stirred for 0.5 h, and then 5,7-dichloro-8-fluoro-2-(methylsulfanyl)pyrido[4,3-d]pyrimidin-4 (3H)-one (1.1 g, 3.93 mmol, 1 eq) was added at 0° C. The mixture was stirred at 25° C. for 1.5 h. On completion, the reaction mixture was quenched by addition of saturated solution of NH₄Cl in water (10 mL) at 0° C. And the residue was diluted with H₂O (50) mL and extracted with EA (30 mL*3). The combined organic layers were dried over Na₂SO₄, filtered, and concentrated under reduced pressure to give a residue. The residue was purified by column chromatography (SiO₂, DCM/MeOH=1/0 to 0/1) to give tert-butyl (2S)-2-({[7-chloro-8-fluoro-4-hydroxy-2-(methylsulfanyl)pyrido[4,3-d]pyrimidin-5-yl]oxy}methyl)piperidine-1-carboxylate (650 mg, 1.42 mmol, 36% yield) as yellow solid. LCMS: m/z 358.9 (M-100+1).

Step 5. A solution of tert-butyl (2S)-2-({[7-chloro-8-fluoro-4-hydroxy-2-(methylsulfanyl)pyrido[4,3-d]pyrimidin-5-yl]oxy}methyl)piperidine-1-carboxylate (500 mg, 1.09 mmol, 1 eq) in POCl₃ (16.50 g, 107.61 mmol, 98.77 eq) was stirred at 80° C. for 1 h. The reaction mixture was concentrated under reduced pressure to remove POCl₃. Then DCM (10 mL) was added, and DIEA (422.42 mg, 3.27 mmol, 3 eq) was added at 0° C. and stirred for 1 h. On completion, the reaction mixture was concentrated under reduced pressure to remove solvent. The residue was purified by column chromatography (SiO₂, Petroleum ether/Ethyl acetate=1/0 to 0/1) to give (8aS)-5-chloro-4-fluoro-2-(methylsulfanyl)-8,8a,9,10,11,12-hexahydro-7-oxa-1,3,6,12a-tetraazabenzo[4,5]cyclohepta[1,2,3-de]naphthalene (180 mg, 0.528 mmol, 49% yield) as white solid. LCMS: m/z 340.9 (M+1).

Step 6. To a solution of (8aS)-5-chloro-4-fluoro-2-(methylsulfanyl)-8,8a,9,10,11,12-hexahydro-7-oxa-1,3,6,12a-tetraazabenzo[4,5]cyclohepta[1,2,3-de]naphthalene (90 mg, 0.264 mmol, 1 eq) in THF (1 mL) and H₂O (0.2 mL) was added K₃PO₄ (168.17 mg, 0.792 mmol, 3 eq), {[2-fluoro-6-(methoxymethoxy)-8-(4,4,5,5-tetramethyl-1,3,2-dioxaborolan-2-yl)naphthalen-1-yl]ethynyl}tri(propan-2-yl)silane (203.03 mg, 0.396 mmol, 1.5 eq) and [2-(2-aminophenyl)phenyl]palladium(1+); bis(1-adamantyl)-butyl-phosphane; methanesulfonate (19.23 mg, 0.0264 mmol, 0.1 eq) under N₂. The mixture was stirred at 80° C. for 2 hr under microwave. On completion, the reaction mixture was quenched with water (20 ml), and extracted with ethyl acetate (10 mL×3). The combined organic phase were dried over anhydrous Na₂SO₄, filtered and concentrated to give a residue. The residue was purified by column chromatography (SiO₂, Petroleum ether/Ethyl acetate=4:1 to 1:1) to give (8aS)-4-fluoro-5-[7-fluoro-3-(methoxymethoxy)-8-{[tri(propan-2-yl)silyl]ethynyl}naphthalen-1-yl]-2-(methylsulfanyl)-8,8a,9,10,11,12-hexahydro-7-oxa-1,3,6,12a-tetraazabenzo[4,5]cyclohepta[1,2,3-de]naphthalene (94 mg, 0.136 mmol, 52% yield) as brown solid. ¹H NMR (400 MHz, CHLOROFORM-d) δ=7.69-7.56 (m, 1H), 7.41-7.32 (m, 1H), 7.30-7.24 (m, 1H), 7.20-7.11 (m, 2H), 5.22-5.13 (m, 2H), 4.40-4.24 (m, 2H), 3.47-3.33 (m, 3H), 2.04-1.90 (m, 2H), 1.88-1.80 (m, 2H), 1.79-1.70 (m, 2H), 1.67-1.56 (m, 2H), 1.55-1.49 (m, 3H), 1.04-0.65 (m, 21H). LCMS: m/z 691.4 (M+1)

Step 7. To a solution of (8aS)-4-fluoro-5-[7-fluoro-3-(methoxymethoxy)-8-{[tri(propan-2-yl)silyl]ethynyl}naphthalen-t-yl]-2-(methylsulfanyl)-8,8a,9,10,11,12-hexahydro-7-oxa-1,3,6,12a-tetraazabenzo[4,5]cyclohepta[1,2,3-de]naphthalene (84 mg, 0.122 mmol, 1 eq) in DCM (1 mL) was added m-CPBA (61.71 mg, 0.304 mmol, 85% purity, 2.5 eq) at 0° C. The mixture was stirred at 25° C. for 1 h. On completion, the reaction mixture was quenched by addition of saturated solution of Na₂SO₃ in water (30 mL) at 0° C., extracted with DCM (10 mL*3). The combined organic layers were dried over Na₂SO₄, filtered, and concentrated under reduced pressure to give a residue. The residue was purified by column chromatography (SiO₂, Petroleum ether/THF=1/0 to 0/1) to give (8aS)-4-fluoro-5-[7-fluoro-3-(methoxymethoxy)-8-{[tri(propan-2-yl)silyl]ethynyl}naphthalen-1-yl]-2-(methanesulfonyl)-8,8a,9,10,11,12-hexahydro-7-oxa-1,3,6,12a-tetraazabenzo[4,5]cyclohepta[1,2,3-de]naphthalene (65 mg, 0.0899 mmol, 74% yield) as brown oil. LCMS: m/z 723.4 (M+1).

Step 8. A mixture of (8aS)-4-fluoro-5-[7-fluoro-3-(methoxymethoxy)-8-{[tri(propan-2-yl)silyl]ethynyl}naphthalen-1-yl]-2-(methanesulfonyl)-8,8a,9,10,11,12-hexahydro-7-oxa-1,3,6,12a-tetraazabenzo[4,5]cyclohepta[1,2,3-de]naphthalene (65 mg, 0.0899 mmol, 1 eq), t-BuOK (30.27 mg, 0.270 mmol, 3 eq) in Tol. (0.5 mL) was degassed and purged with N₂ for 3 times at 0° C., and then [(2R,7aS)-2-fluorotetrahydro-1H-pyrrolizin-7a(5H)-yl]methanol (42.94 mg, 0.270 mmol, 3 eq) was added at 0° C., the mixture was stirred at 25° C. for 1 h under N₂ atmosphere. On completion, the reaction mixture was diluted with water (20 mL) and extracted with ethyl acetate (10 mL×3). The combined organic phase was dried over anhydrous sodium sulfate, filtered and concentrated to give a residue. The residue was purified by column chromatography (SiO₂, Petroleum ether/Ethyl acetate=4:1 to 0:1) to give (8aS)-4-fluoro-5-[7-fluoro-3-(methoxymethoxy)-8-{[tri(propan-2-yl)silyl]ethynyl}naphthalen-1-yl]-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene (75 mg, 0.0888 mmol, 99% yield) as white solid. LCMS: m/z 802.4 (M+1).

Step 9. To a solution of (8aS)-4-fluoro-5-[7-fluoro-3-(methoxymethoxy)-8-{[tri(propan-2-yl)silyl]ethynyl}naphthalen-1-yl]-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene (68 mg, 0.0848 mmol, 1 eq) in DCM (1 mL) was added HCl/dioxane (4 M, 0.2 mL, 9.44 eq). The mixture was stirred at 25° C. for 0.5 h. On completion, the reaction mixture was concentrated under reduced pressure t to give 6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-5-{[tri(propan-2-yl)silyl]ethynyl}naphthalen-2-ol (55 mg, crude). LCMS: m/z 758.3 (M+1).

Step 10. To a solution of 6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-5-{[tri(propan-2-yl)silyl]ethynyl}naphthalen-2-ol (55 mg, 0.0726 mmol, 1 eq) in DMSO (0.5 mL) was added CsF (66.14 mg, 0.435 mmol, 6 eq). The mixture was stirred at 25° C. for 1 h. On completion, the mixture was filtered. The filtrate was purified by prep-HPLC (column: Phenomenex luna C18 150*25 mm*10 um; mobile phase: [water(FA)-ACN]; B %: 22%-42%, 58 min) to give 5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol (16.59 mg, 0.0256 mmol, 35.30% yield as formic acid salt) as brown gum.

¹H NMR (400 MHz, DMSO-d₆) δ=10.29-9.99 (m, 1H), 7.99-7.93 (m, 1H), 7.46 (t, J=8.8 Hz, 1H), 7.37 (d, J=2.4 Hz, 1H), 7.18-7.12 (m, 1H), 5.38-5.19 (m, 1H), 4.52-4.38 (m, 2H), 4.06 (s, 2H), 4.01-3.91 (m, 2H), 3.13-3.07 (m, 2H), 3.03 (br s, 2H), 2.87-2.79 (m, 1H), 2.15-2.05 (m, 2H), 2.03-1.98 (m, 1H), 1.95-1.66 (m, 10H). LCMS: m/z 602.2 (M+1).

### Preparation of 5-ethyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol (Ex. 2)

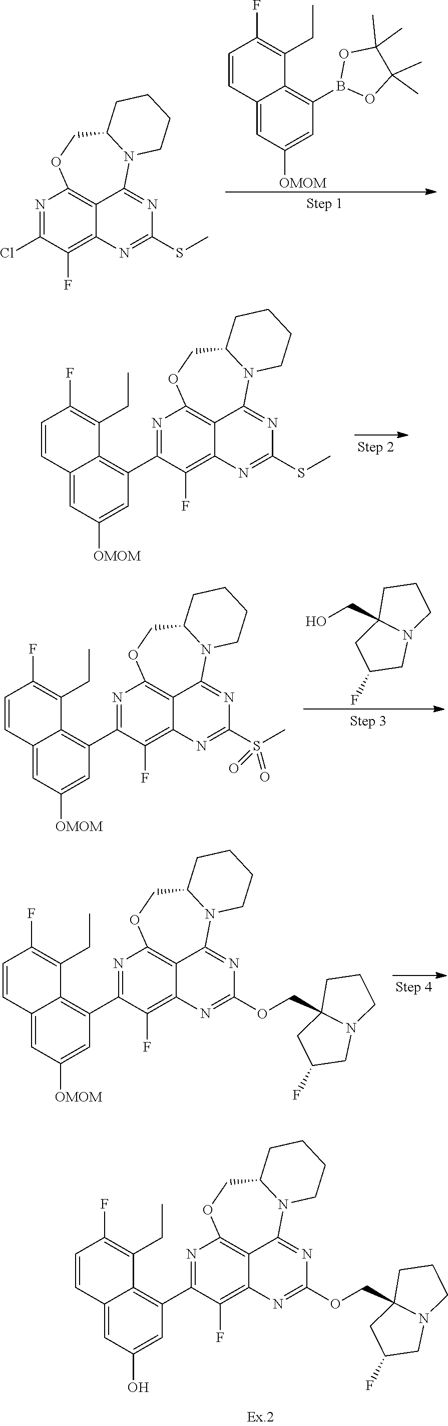

Step 1. To a solution of (8aS)-5-chloro-4-fluoro-2-(methylsulfanyl)-8,8a,9,10,11,12-hexahydro-7-oxa-1,3,6,12a-tetraazabenzo[4,5]cyclohepta[1,2,3-de]naphthalene (85 mg, 0.249 mmol, 1 eq) in H₂O (0.2 mL) and dioxane (1 mL) was added 2-[8-ethyl-7-fluoro-3-(methoxymethoxy)naphthalen-1-yl]-4,4,5,5-tetramethyl-1,3,2-dioxaborolane (134.77 mg, 0.374 mmol, 1.5 eq), K₃PO₄ (158.82 mg, 0.748 mmol, 3 eq) and [2-(2-aminophenyl)phenyl]palladium(1+); bis(1-adamantyl)-butyl-phosphane; methanesulfonate (18.16 mg, 0.0249 mmol, 0.1 eq). The mixture was stirred at 80° C. for 2 h. On completion, the mixture was quenched with water (20 mL) and extracted with ethyl acetate (25 mL×3), the combined organic phase was dried over anhydrous sodium sulfate, filtered, and concentrated to give a residue. The residue was purified by column chromatography (SiO₂, Petroleum ether/Ethyl acetate=4:1 to 1:1) to give (8aS)-5-[8-ethyl-7-fluoro-3-(methoxymethoxy)naphthalen-1-yl]-4-fluoro-2-(methylsulfanyl)-8,8a,9,10,11,12-hexahydro-7-oxa-1,3,6,12a-tetraazabenzo[4,5]cyclohepta[1,2,3-de]naphthalene (80 mg, 0.149 mmol, 60% yield) as yellow solid. LCMS: m/z 539.3 (M+1).

Step 2. To a solution of (8aS)-5-[8-ethyl-7-fluoro-3-(methoxymethoxy)naphthalen-1-yl]-4-fluoro-2-(methylsulfanyl)-8,8a,9,10,11,12-hexahydro-7-oxa-1,3,6,12a-tetraazabenzo[4,5]cyclohepta[1,2,3-de]naphthalene (72 mg, 0.134 mmol, 1 eq) in DCM (1 mL) was added m-CPBA (67.85 mg, 0.334 mmol, 85% purity, 2.5 eq) at 0° C. The mixture was stirred at 25° C. for 1 h. On completion, the reaction mixture was quenched with saturated solution of Na₂SO₃ in water (20 mL) at 0° C. and extracted with DCM (10 mL*3). The combined organic layers were dried over Na₂SO₄, filtered, and concentrated under reduced pressure to give a residue. The residue was purified by column chromatography (SiO₂, Petroleum ether/THF=1/0 to 0/1) to give (8aS)-5-[8-ethyl-7-fluoro-3-(methoxymethoxy)naphthalen-1-yl]-4-fluoro-2-(methanesulfonyl)-8,8a,9,10,11,12-hexahydro-7-oxa-1,3,6,12a-tetraazabenzo[4,5]cyclohepta[1,2,3-de]naphthalene (45 mg, 0.0789 mmol, 59% yield) as yellow oil. LCMS: m/z 571.3 (M+1).

Step 3. A mixture of [(2R,7aS)-2-fluorotetrahydro-1H-pyrrolizin-7a(5H)-yl]methanol (30.13 mg, 0.189 mmol, 3 eq), t-BuOK (21.24 mg, 0.189 mmol, 3 eq) in Tol. (0.5 mL) was degassed and purged with N₂ for 3 times at 0° C., and then (8aS)-5-[8-ethyl-7-fluoro-3-(methoxymethoxy)naphthalen-1-yl]-4-fluoro-2-(methanesulfonyl)-8,8a,9,10,11,12-hexahydro-7-oxa-1,3,6,12a-tetraazabenzo[4,5]cyclohepta[1,2,3-de]naphthalene (36 mg, 0.0631 mmol, 1 eq) was added at 0° C., and then the mixture was stirred at 25° C. for 2 h under N₂ atmosphere. On completion, the mixture was quenched with water (20 mL) and extracted with ethyl acetate (25 mL×3), the combined organic phase was dried over anhydrous sodium sulfate, filtered, and concentrated to give a residue. The residue was purified by column chromatography (SiO₂, Petroleum ether/Ethyl acetate=4:1 to 1:1) to give (8aS)-5-[8-ethyl-7-fluoro-3-(methoxymethoxy)naphthalen-1-yl]-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene (55 mg, 0.0626 mmol, 99% yield, 74% purity) as white solid. LCMS: m/z 650.3 (M+1).

Step 4. To a solution of (8aS)-5-[8-ethyl-7-fluoro-3-(methoxymethoxy)naphthalen-1-yl]-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene (50 mg, 0.0770 mmol, 1 eq) in DCM (0.5 mL) was added HCl/dioxane (4 M, 19.24 uL, 1 eq). The mixture was stirred at 25° C. for 1 h. On completion, the mixture was concentrated in vacuum to give the residue. The residue was purified by prep-HPLC (column: Phenomenex luna C18 150*25 mm*10 um; mobile phase: [water(FA)-ACN]; B %: 27%-47%, 58 min) to give 5-ethyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol (9.48 mg, 0.0146 mmol, 18.90% yield as a formic acid) as brown solid. ¹H NMR (400 MHz, DMSO-d₆) δ=7.74 (dd, J=6.0, 9.0 Hz, 1H), 7.40-7.24 (m, 2H), 7.00 (dd, J=2.6, 18.1 Hz, 1H), 5.40-5.05 (m, 2H), 4.57-4.40 (m, 2H), 4.14 (d, J=10.4 Hz, 1H), 4.03-3.90 (m, 2H), 3.16-2.95 (m, 5H), 2.90-2.77 (m, 1H), 2.35-2.22 (m, 2H), 2.16-2.11 (m, 1H), 2.00 (br s, 1H), 1.92-1.60 (m, 8H), 1.57-1.43 (m, 1H), 0.87-0.73 (m, 3H). LCMS: 606.2 (M+1).

### General Method D: Preparation of (8aR,9R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol (Ex. 25) and (8aS,9S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol (Ex. 103)

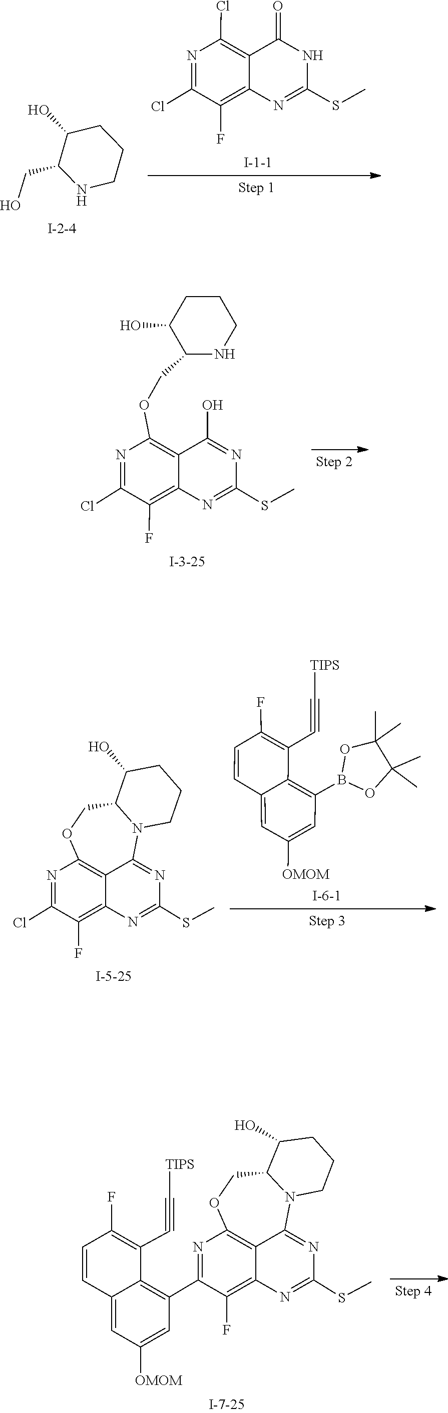

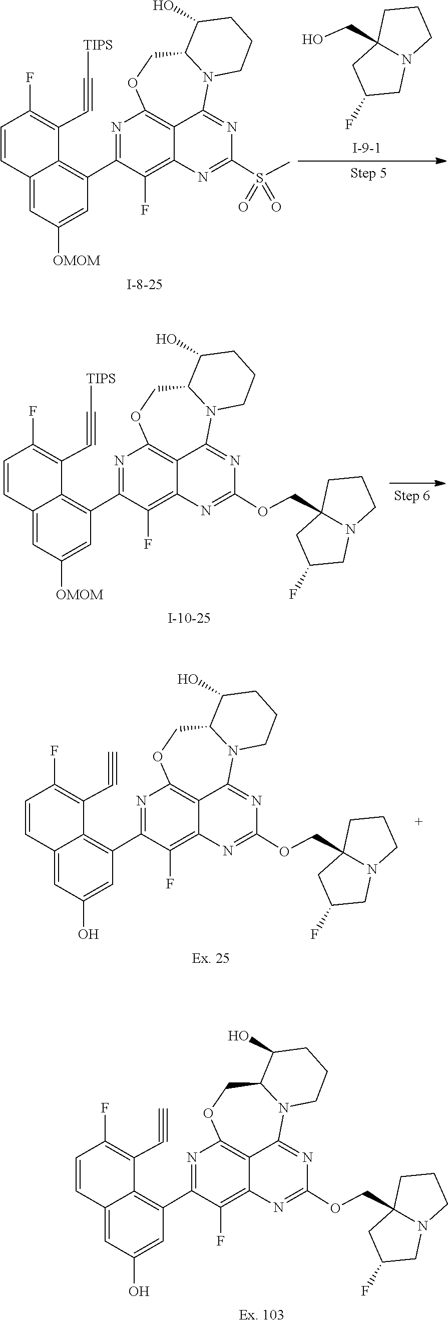

Step 1. To a solution of I-2-2 (1 eq) in DMF or THE was added NaH (2 eq) at 0° C. for 10 minutes. Then I-1-1 (0.8 eq) was added to the mixture. The mixture was stirred at 25° C. for 16 h. On completion, the mixture was quenched with water. The mixture was then purified by reverse phase (Acetonitrile in H₂O) to give I-3-25.

Step 2. To a solution of I-3-25 (1 eq) in Acetonitrile was added DIPEA (9 eq) and POCl₃ (3 eq). The mixture was stirred at 25° C. for 2 h. On completion, the mixture was quenched with water and extracted with EtOAc. The combined organic layer was dried over anhydrous Na₂SO₄, filtered and the filtrate was concentrated in vacuum to give I-5-25.

Step 3. To a solution of I-5-25 (1 eq), I-6-1 (1.5 eq) in Dioxane-H₂O (5:1) was added K₃PO₄ (3 eq) and cataCXium® A Pd G3 (0.1 eq). The mixture was stirred at 80° C. for 2 h under N₂. On completion, the mixture was dried over anhydrous Na₂SO₄, filtered and the filtrate was concentrated in vacuum to give a crude residue, which was purified by combi flash (silica gel column, EtOAc in Hexanes) to give I-7-25.

Step 4. To a solution of I-7-25 (1 eq) in DCM was added m-CPBA (2.5 eq) at 0° C. and the mixture was stirred at 25° C. for 20 minutes. On completion, the mixture was diluted with water and extracted with DCM. The combined organic layer was dried over anhydrous Na₂SO₄, filtered and the filtrate was concentrated in vacuum to give I-8-25.

Step 5. To a solution of I-9-1 (3 eq) in Toluene was added t-BuOK (3 eq) at 0° C. and stirred for 20 minutes. A solution of I-8-25 (1 eq) in Toluene was then added, and the mixture was stirred at 25° C. for 2 h. On completion, the mixture was diluted with water and extracted with DCM. The combined organic layer was dried over anhydrous Na₂SO₄, filtered and filtrate was concentrated in vacuum to give I-10-25.

Step 6. To a solution of I-10-25 (1 eq) in DCM was added HCl/Dioxane (4 M, 7.44 eq). The mixture was stirred at 25° C. for 20 minutes. On completion, the mixture was concentrated in vacuum. To a solution of the above crude residue (1 eq) in DMSO was added CsF (3 eq). The mixture was stirred at 25° C. for 1 h. On completion, the mixture was filtered, and the filtrate was concentrated in vacuum to give a crude residue, which was purified by Prep-HPLC (column: Waters xbridge; mobile phase: [water (NH₄HCO₃)-ACN]) to give a mixture of diastereomers. The mixture was purified by SFC separation (column: REGIS (R,R)WHELK-O1; mobile phase: [Heptane-(IPA:ACN) (0.1% NH₃·H₂O)]) to give a residue, which was purified by Prep-HPLC (column: Phenomenex luna C18; mobile phase: [water(FA)-ACN] to furnish Ex. 25 and Ex. 103.

### General Method E: Preparation of 4-[(9S)-9-cyclopropyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethynyl-6-fluoronaphthalen-2-ol (Ex. 31)

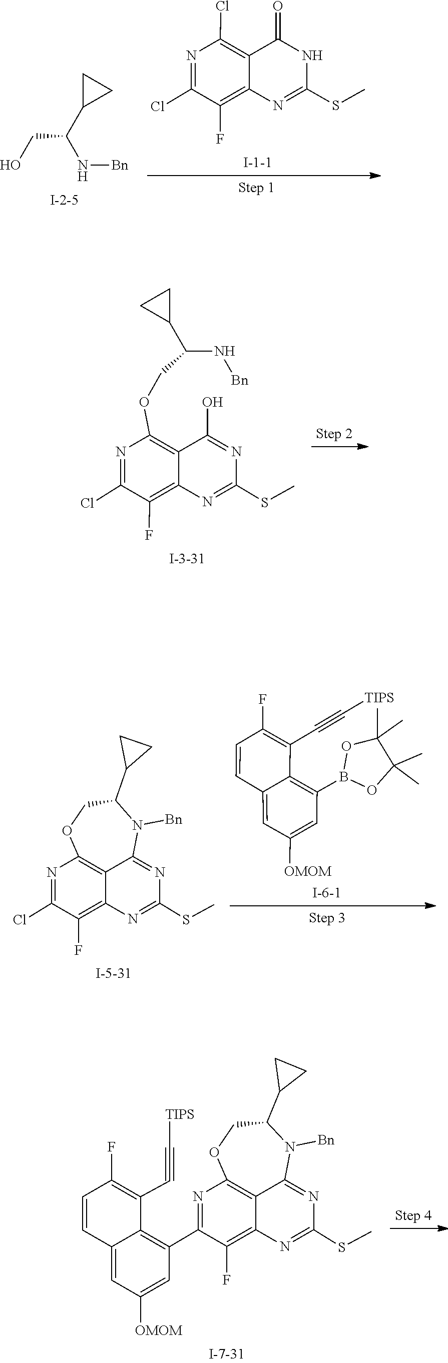

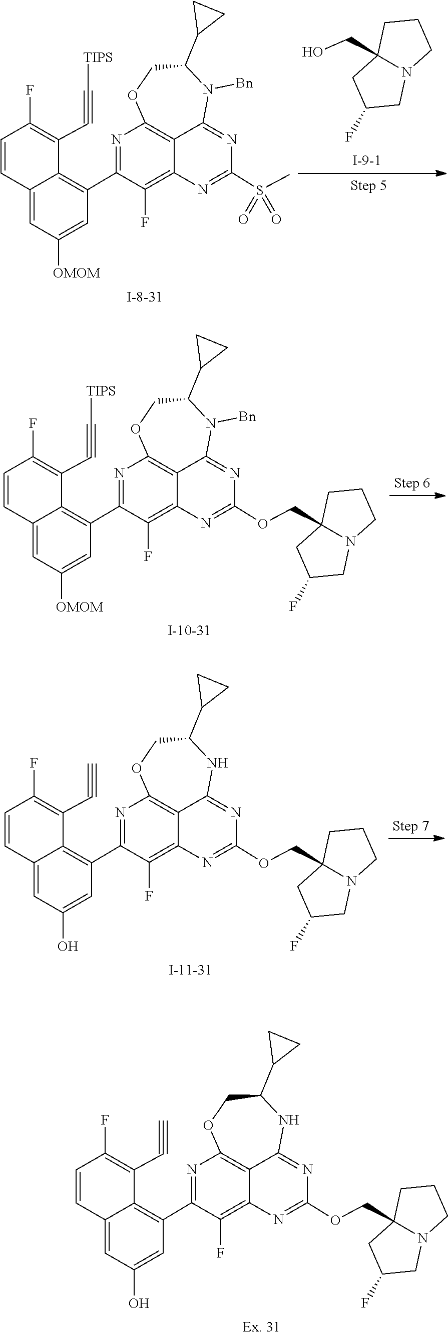

Step 1. To a solution of I-2-5 in THF was added NaH (2 eq) at 0° C. for 0.5 h. Then I-1-1 (1 eq) was added to the mixture and stirred at 25° C. for 2 h. On completion, the mixture was quenched with sat. NH4Cl aqueous and extracted with Ethyl acetate. The combined organic layer was washed with brine, dried over Na₂SO₄, filtered, and concentrated to give a residue. The residue was purified by column chromatography (SiO₂, Petroleum ether/THF) to give I-3-31.

Step 2. To a solution of I-5-31 (1 eq) in Acetonitrile was added POCl₃ (2 eq) and DIPEA (6 eq). The mixture was stirred at 25° C. for 1 h. On completion, the mixture was concentrated in vacuum remove POCl₃. The residue was purified by column chromatography (SiO₂, Petroleum ether/Ethyl acetate) to furnish I-5-31.

Step 3. A mixture of I-5-31 (1 eq), I-6-1 (1.5 eq), K₃PO₄ (458 mg, 2.16 mmol, 3 eq) in H₂O (1 mL) and dioxane (5 mL) was degassed and cataCXium® A Pd G3 (0.1 eq.) was added. The mixture was then purged with N₂ 3 times and stirred at 80° C. for 2 h in microwave under N₂ atmosphere. On completion, the reaction mixture was diluted with water and was extracted with EtOAc. The combined organic layer was washed with brine, dried over Na₂SO₄, filtered, and concentrated to give a residue. The residue was purified by column chromatography (SiO₂, Petroleum ether/Ethyl acetate) to give I-7-31.

Step 4. To a solution of I-7-31 (1 eq) in DCM was added m-CPBA (2.5 eq). The mixture was stirred at 25° C. for 1 h. On completion, the reaction mixture was quenched by addition sat. Na₂SO₃ aqueous at 0° C., and then diluted with H₂O and extracted with DCM. The combined organic layers were dried over Na₂SO₄, filtered, and concentrated under reduced pressure to give I-8-31.

Step 5. To a solution of I-8-31 (3 eq) in toluene was added t-BuOK (3 eq), the mixture was degassed and purged with N₂ 3 times for 30 min. Then I-9-1 (1 eq) was added and stirred at 25° C. for 1 h. On completion, the reaction mixture was diluted with water and was extracted with EtOAc. The combined organic layer was washed with brine, dried over Na₂SO₄, filtered, and concentrated to give a residue. The residue was purified by column chromatography (SiO₂, Petroleum ether/Ethyl acetate) to afford I-10-31.

Step 6. To a solution of I-10-31 (1 eq) in i-PrOH was added Pd/C (10% purity, 0.125 eq), Pd(OH)₂ (10% purity, 0.095 eq) and 1,1,2-trichloroethane (2 eq) was degassed and purged with H₂ (15 psi) 3 times, and then the mixture was stirred at 25° C. for 12 h under H₂ atmosphere. On completion, the mixture was filtered with diatomaceous earth. The residue was purified by column chromatography (SiO₂, DCM/MeOH) to furnish I-11-31.

Step 7. To a solution of I-11-31 (1 eq) in DCM was added HCl/dioxane (4 M, 42 eq). The mixture was stirred at 25° C. for 1 h. On completion, the mixture was concentrated in vacuum. The above crude residue was dissolved in DMSO, and CsF (5 eq) was added. The mixture was stirred at 25° C. for 30 min. On completion, the mixture was filtered, and the filtrate was purified by Prep-HPLC purification to afford Ex. 31.

General Method F: Preparation of (9S)-9-ethyl-4-fluoro-5-[7-fluoro-3-(methoxymethoxy)-8-{[tri(propan-2-yl)silyl]ethynyl}naphthalen-1-yl]-10-methyl-2-(methylsulfanyl)-9,10-dihydro-8H-7-oxa-1,3,6,10-tetraazacyclohepta[1,2,3-de]naphthalene (I-7-96):

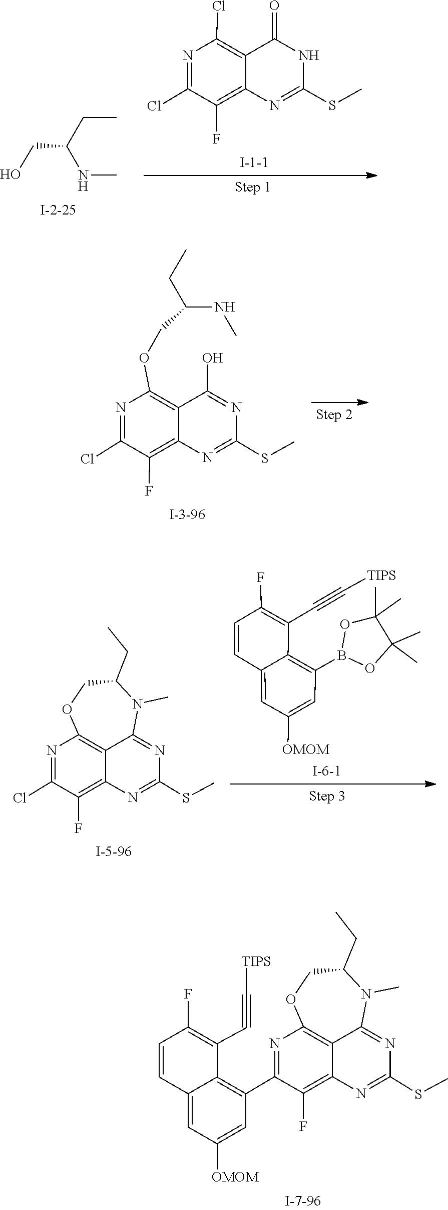

Step 1. To a solution of I-2-25 (1 eq) in THF was added NaH (60% purity, 2.5 eq) and 5,7-dichloro-8-fluoro-2-methylsulfanyl-3H-pyrido[4,3-d]pyrimidin-4-one (I-1-1, 1 eq). The mixture was stirred at 0° C. for 2 hours. On completion, the mixture was quenched with water and extracted with ethyl acetate. The combined organic phase was dried over anhydrous sodium sulfate, filtered, and concentrated to give a residue. The residue was purified by column chromatography to afford I-3-96.

Step 2. To a solution of I-3-96 (1 eq) in Acetonitrile was added NMI (12 eq) and TCFH (3 eq). The mixture was stirred at 25° C. for 14 hours. On completion, the reaction mixture was partitioned between ethyl acetate and water, and the combined organic phase was dried over anhydrous sodium sulfate, filtered, and concentrated to give a residue. The residue was purified by column chromatography to furnish I-5-96.

Step 3. A mixture of I-5-96 (1 eq), I-6-1 (1.1 eq), cataCXium© A Pd G3 (0.1 eq), and K₃PO₄ (3 eq) in THF and H₂O was degassed and purged with N₂ 3 times, and then the mixture was stirred at 80° C. for 12 hours under N₂ atmosphere. On completion, the reaction mixture was partitioned between ethyl acetate and water. The combined organic phase was dried over anhydrous sodium sulfate, filtered, and concentrated to give a residue. The residue was purified by column chromatography to give I-7-96.

### General Method H: Preparation of 2-chloro-1-fluoro-11-(methylthio)-5,5a,6,7,8,9-hexahydro-4H-3,9a,10,12-tetraazabenzo[4,5]cyclohepta[1,2,3-de]naphthalene (I-121b)

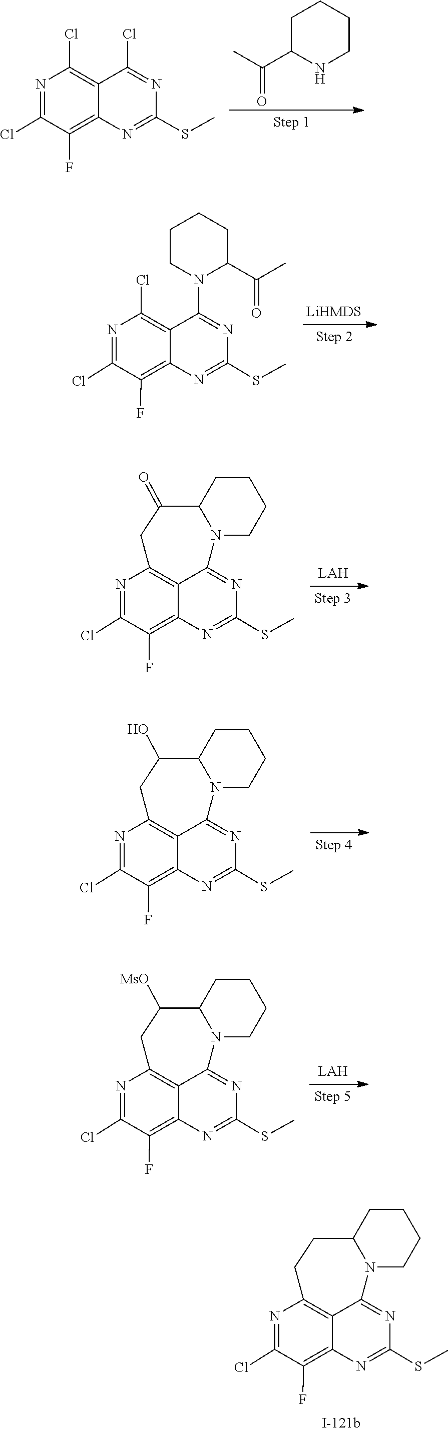

Step 1. To a solution of 4,5,7-trichloro-8-fluoro-2-(methylthio)pyrido[4,3-d]pyrimidine (0.737 g, 2.47 mmol, 1 eq) and 1-(piperidin-2-yl)ethan-1-one hydrochloride (404 mg, 2.47 mmol, 1 eq) in DCM (11.5 mL) at 0° C. was added DIPEA (638 mg, 4.94 mmol, 860 μL, 2 eq) at 0° C. The mixture was slowly warmed to ambient temperature over 2 hours then quenched with saturated NaHCO₃ solution (5 mL). The mixture was extracted with DCM (3×10 mL) and combined extracts were dried with Na₂SO₄ and concentrated under reduced pressure. Flash chromatography, (automated system, 80 g Silica, 5-30% ethyl acetate in hexane) provided 1-(1-(5,7-dichloro-8-fluoro-2-(methylthio)pyrido[4,3-d]pyrimidin-4-yl)piperidin-2-yl)ethan-1-one (785 mg, 2.02 mmol, 82% yield) as a white solid: LCMS: m/z 389.1 (M+1).

Step 2. To a solution of LiHMDS (1M, 3.45 mL, 2.7 eq) in THE (15 mL) at −23° C. was added 1-(1-(5,7-dichloro-8-fluoro-2-(methylthio)pyrido[4,3-d]pyrimidin-4-yl)piperidin-2-yl)ethan-1-one (500 mg, 1.28 mmol in 3 mL THF, 1 eq) dropwise by syringe pump over 75 minutes. The mixture was stirred at −23° C. for 30 minutes more than quenched by addition of saturated solution of NH₄Cl (25 mL). The reaction mixture was warmed to ambient temperature and extracted with ethyl acetate (3×20 mL) and combined extracts were dried with bine (25 mL), then Na₂SO₄ and concentrated under reduced pressure. Flash chromatography, (automated system, 40 g Silica, 0-20% ethyl acetate in hexane) provided 2-chloro-1-fluoro-11-(methylthio)-6,7,8,9-tetrahydro-4H-3,9a,10,12-tetraazabenzo[4,5]cyclohepta[1,2,3-de]naphthalen-5 (5aH)-one (226.5 mg, 0.642 mmol, 50% yield) as a white crystalline solid: LCMS: m/z 353.1 (M+1). ¹H NMR (499 MHz, DMSO-d6) δ ppm 4.44-4.51 (m, 1H) 4.33 (br s, 1H) 4.28 (d, J=13.14 Hz, 1H) 4.17 (d, J=13.69 Hz, 1H) 3.63 (ddd, J=13.14, 8.76, 4.38 Hz, 1H) 2.58 (s, 3H) 1.96-2.03 (m, 1H) 1.71-1.82 (m, 3H) 1.54-1.70 (m, 2H).

Step 3. To a solution of 2-chloro-1-fluoro-11-(methylthio)-6,7,8,9-tetrahydro-4H-3,9a,10,12-tetraazabenzo[4,5]cyclohepta[1,2,3-de]naphthalen-5 (5aH)-one (33.8 mg, 95.8 μmol, 1 eq) in THE (950 μL) at −78° C. was added LAH (2 M, 96 μL, 2 eq). The mixture was stirred at −78° C. and stirred for 3 h. The mixture was quenched at −78° C. by addition of 1:1 ethyl acetate in hexane (0.5 mL) followed by water (1 mL) then 2M HCl (0.2 mL). The mixture was warmed to ambient temperature then extracted with ethyl acetate (3×5 mL) and combined extracts were dried with Na₂SO₄ and concentrated under reduced pressure. Flash chromatography, (automated system, 12 g Silica, 10-70% ethyl acetate in hexane) provided 2-chloro-1-fluoro-11-(methylthio)-5,5a,6,7,8,9-hexahydro-4H-3,9a,10,12-tetraazabenzo[4,5]cyclohepta[1,2,3-de]naphthalen-5-ol (25.21 mg, 71.05 μmol, 74.16% yield) as a white crystalline solid (˜2:1 mixture of trans:cis): LCMS: m/z 355.1 (M+1). Analytical data for separately isolated cis isomer: ¹H NMR (499 MHz, DMSO-d6) δ ppm 5.42 (d, J=5.48 Hz, 1H) 4.28-4.36 (m, 1H) 4.04 (dt, J=13.14, 5.48 Hz, 1H) 3.85-3.93 (m, 1H) 3.46 (dt, J=9.86, 3.83 Hz, 1H) 3.23 (dd, 1=13.69, 4.93 Hz, 1H) 3.04 (dd, J=13.14, 8.76 Hz, 1H) 2.53 (s, 3H) 1.87-1.94 (m, 1H) 1.78-1.87 (m, 1H) 1.69-1.78 (m, 2H) 1.58-1.65 (m, 1H) 1.35-1.49 (m, 1H).

Step 4. To a solution of 2-chloro-1-fluoro-11-(methylthio)-5,5a,6,7,8,9-hexahydro-4H-3,9a,10,12-tetraazabenzo[4,5]cyclohepta[1,2,3-de]naphthalen-5-ol (30 mg, 84.7 μmol, 1 eq) and Triethylamine (55 mg, 538 μmol, 75 μL) in THF (1 mL) at 0° C. was added methanesulfonyl chloride (22.2 mg, 193.4 μmol, 15 μL) dropwise. The reaction mixture was stirred for 1 hour at 0° C. then quenched with water (3 mL). The mixture extracted with DCM (2×5 mL). Combined extracts were dried with Na₂SO₄ and concentrated under reduced pressure. Flash chromatography, (automated system, 12 g Silica, 10-50% ethyl acetate in hexane) gave 2-chloro-1-fluoro-11-(methylthio)-5,5a,6,7,8,9-hexahydro-4H-3,9a,10,12-tetraazabenzo[4,5]cyclohepta[1,2,3-de]naphthalen-5-yl methanesulfonate (35.85 mg, 82.8 μmol, 97.8% yield) as a white solid: LCMS: m/z 433.1 (M+1).

Step 5. To a solution of 2-chloro-1-fluoro-11-(methylthio)-5,5a,6,7,8,9-hexahydro-4H-3,9a,10,12-tetraazabenzo[4,5]cyclohepta[1,2,3-de]naphthalen-5-yl methanesulfonate (5.22 mg, 12.1 μmol) in THF (1 mL) at −78° C. was added LAH (2 M, 60 μL). The mixture was stirred at −78° C. for 1 hour then warmed to 0° C. and stirred for a further 2 hr. The mixture was cooled to −78° C. then quenched by addition of ethyl acetate (500 μL) then 2M HCl (350 μL). The mixture was extracted with ethyl acetate (3×5 mL) and combined extracts were washed with saturated NaHCO₃ (15 mL) dried with Na₂SO₄ and concentrated under reduced pressure. Flash chromatography, (automated system, 12 g Silica, 0-25% ethyl acetate in hexane) gave 2-chloro-1-fluoro-11-(methylthio)-5,5a,6,7,8,9-hexahydro-4H-3,9a,10,12-tetraazabenzo[4,5]cyclohepta [1,2,3-de]naphthalene (0.84 mg, 1.74 μmol, 14.4% yield, 70% purity) as a white solid: LCMS: m/z 339.1 (M+1). ¹H NMR (499 MHz, CHLOROFORM-d) δ ppm 4.73 (br d, J=12.59 Hz, 1H) 3.71-3.85 (m, 1H) 3.46 (br t, J=9.31 Hz, 1H) 3.11-3.31 (m, 2H) 2.61 (s, 3H) 2.41-2.49 (m, 2H) 1.82-2.09 (m, 5H) 1.64-1.75 (m, 1H) 1.48-1.60 (m, 1H).

Compounds of the disclosure (or intermediates used for the synthesis of such compounds) were prepared using the corresponding methods and intermediates as shown in the table below.

| | | Solvent | | |
|---|---|---|---|---|
| General | | used in | Ex. | |
| Method | Intermediate | Step 1 | # | Structure |
| D | 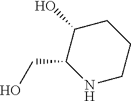 | DMF | 25 | 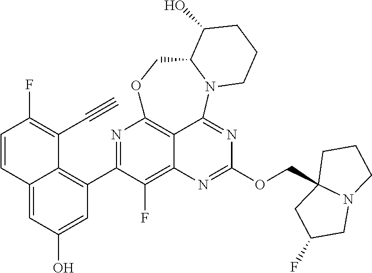 [SMILES: C#Cc1c(F)ccc2cc(O)cc(-c3nc4c5c(nc(OC[C@@]67CCCN6C[C@H](F)C7)nc5c3F)N3CCC[C@@H](O)[C@H]3CO4)c12] |
| D | 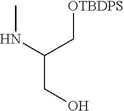 | THF | 27 | 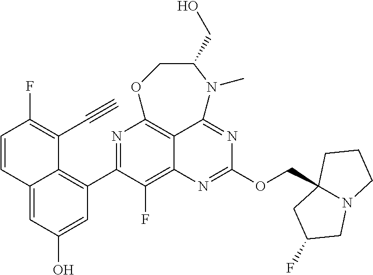 [SMILES: C#Cc1c(F)ccc2cc(O)cc(-c3nc4c5c(nc(OC[C@@]67CCCN6C[C@H](F)C7)nc5c3F)N(C)[C@@H](CO)CO4)c12] |
| D | 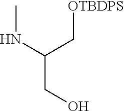 | THF | 28 | 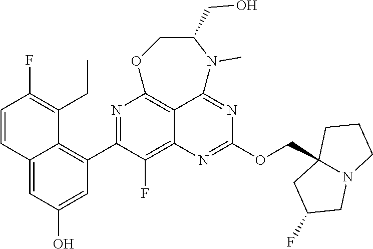 [SMILES: CCc1c(F)ccc2cc(O)cc(-c3nc4c5c(nc(OC[C@@]67CCCN6C[C@H](F)C7)nc5c3F)N(C)[C@@H](CO)CO4)c12] |
| D | 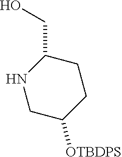 | DMF | 29 | 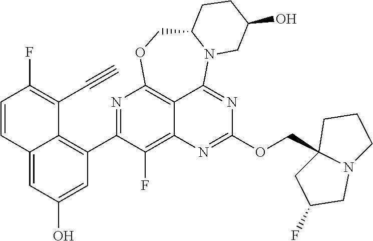 [SMILES: C#Cc1c(F)ccc2cc(O)cc(-c3nc4c5c(nc(OC[C@@]67CCCN6C[C@H](F)C7)nc5c3F)N3C[C@H](O)CC[C@H]3CO4)c12] |
| D | 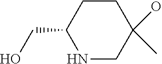 | DMF | 30 | 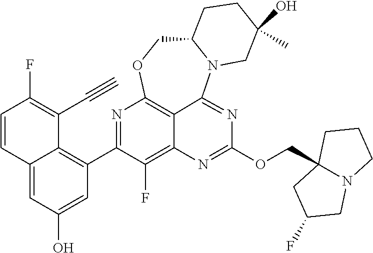 [SMILES: C#Cc1c(F)ccc2cc(O)cc(-c3nc4c5c(nc(OC[C@@]67CCCN6C[C@H](F)C7)nc5c3F)N3C[C@](C)(O)CC[C@H]3CO4)c12] |
| E | 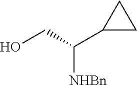 | THF | 31 | 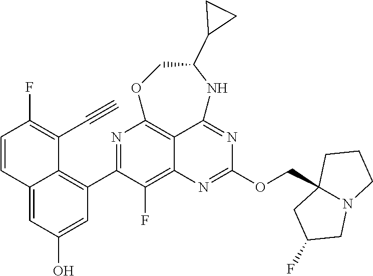 [SMILES: C#Cc1c(F)ccc2cc(O)cc(-c3nc4c5c(nc(OC[C@@]67CCCN6C[C@H](F)C7)nc5c3F)N[C@@H](C3CC3)CO4)c12] |
| D | 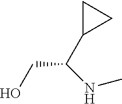 | THF | 32 | 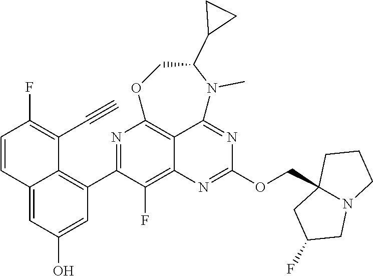 [SMILES: C#Cc1c(F)ccc2cc(O)cc(-c3nc4c5c(nc(OC[C@@]67CCCN6C[C@H](F)C7)nc5c3F)N(C)[C@@H](C3CC3)CO4)c12] |
| E | 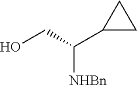 | THF | 33 | 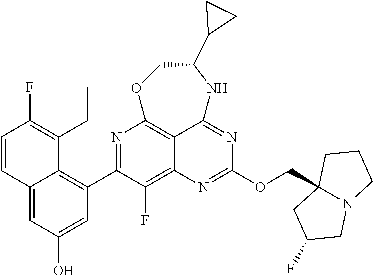 [SMILES: CCc1c(F)ccc2cc(O)cc(-c3nc4c5c(nc(OC[C@@]67CCCN6C[C@H](F)C7)nc5c3F)N[C@@H](C3CC3)CO4)c12] |
| E | 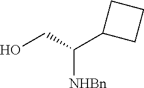 | THF | 35 | 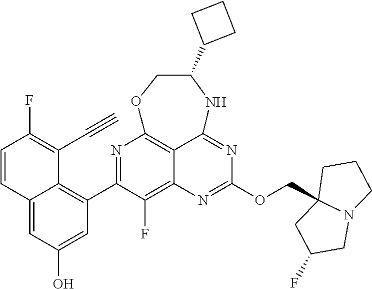 [SMILES: C#Cc1c(F)ccc2cc(O)cc(-c3nc4c5c(nc(OC[C@@]67CCCN6C[C@H](F)C7)nc5c3F)N[C@@H](C3CCC3)CO4)c12] |
| A | 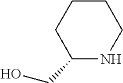 | DMF | 40 | 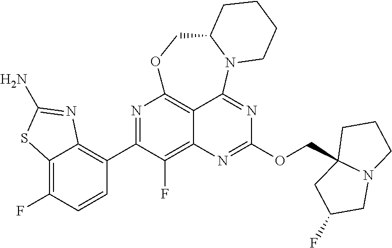 [SMILES: Nc1nc2c(-c3nc4c5c(nc(OC[C@@]67CCCN6C[C@H](F)C7)nc5c3F)N3CCCC[C@H]3CO4)ccc(F)c2s1] |
| A | 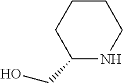 | DMF | 41 | 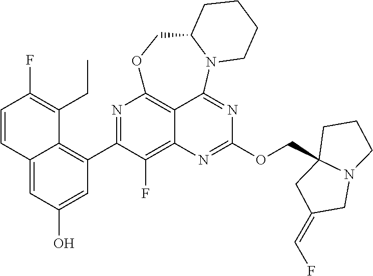 [SMILES: CCc1c(F)ccc2cc(O)cc(-c3nc4c5c(nc(OC[C@@]67CCCN6C/C(=C\F)C7)nc5c3F)N3CCCC[C@H]3CO4)c12] |
| A | 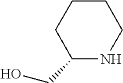 | DMF | 43 | 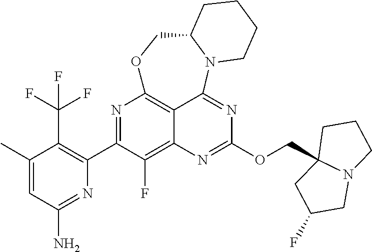 [SMILES: Cc1cc(N)nc(-c2nc3c4c(nc(OC[C@@]56CCCN5C[C@H](F)C6)nc4c2F)N2CCCC[C@H]2CO3)c1C(F)(F)F] |
| A | 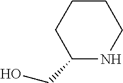 | DMF | 44 | 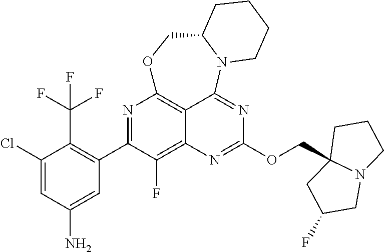 [SMILES: Nc1cc(Cl)c(C(F)(F)F)c(-c2nc3c4c(nc(OC[C@@]56CCCN5C[C@H](F)C6)nc4c2F)N2CCCC[C@H]2CO3)c1] |
| D | 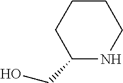 | THF | 45 | 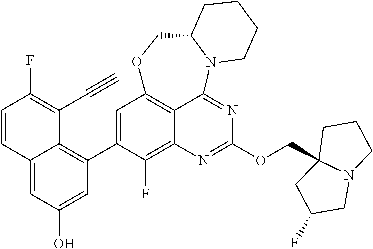 [SMILES: C#Cc1c(F)ccc2cc(O)cc(-c3cc4c5c(nc(OC[C@@]67CCCN6C[C@H](F)C7)nc5c3F)N3CCCC[C@H]3CO4)c12] |
| E | 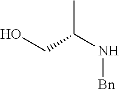 | THF | 46 | 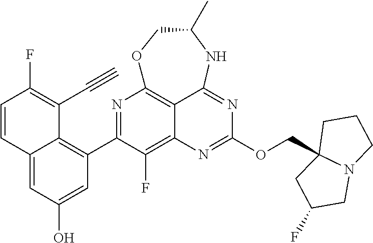 [SMILES: C#Cc1c(F)ccc2cc(O)cc(-c3nc4c5c(nc(OC[C@@]67CCCN6C[C@H](F)C7)nc5c3F)N[C@@H](C)CO4)c12] |
| E | 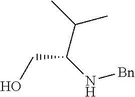 | THF | 47 | 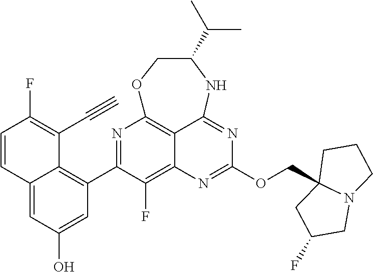 [SMILES: C#Cc1c(F)ccc2cc(O)cc(-c3nc4c5c(nc(OC[C@@]67CCCN6C[C@H](F)C7)nc5c3F)N[C@@H](C(C)C)CO4)c12] |
| E | 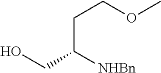 | THF | 48 | 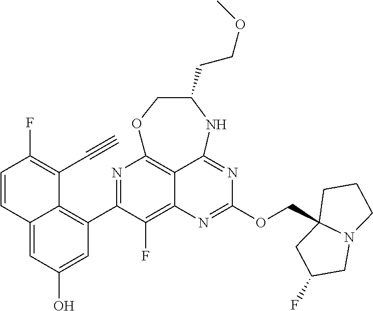 [SMILES: C#Cc1c(F)ccc2cc(O)cc(-c3nc4c5c(nc(OC[C@@]67CCCN6C[C@H](F)C7)nc5c3F)N[C@@H](CCOC)CO4)c12] |
| D | 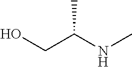 | THF | 50 | 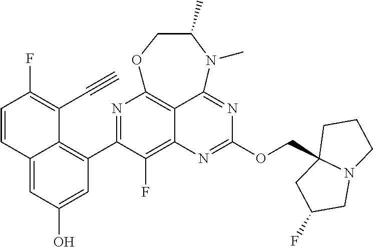 [SMILES: C#Cc1c(F)ccc2cc(O)cc(-c3nc4c5c(nc(OC[C@@]67CCCN6C[C@H](F)C7)nc5c3F)N(C)[C@@H](C)CO4)c12] |
| D | 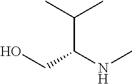 | THF | 51 | 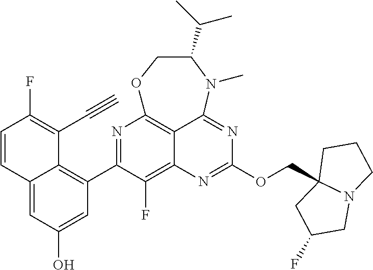 [SMILES: C#Cc1c(F)ccc2cc(O)cc(-c3nc4c5c(nc(OC[C@@]67CCCN6C[C@H](F)C7)nc5c3F)N(C)[C@@H](C(C)C)CO4)c12] |
| D | 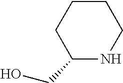 | DMF | 52 | 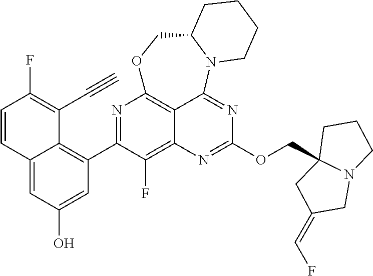 [SMILES: C#Cc1c(F)ccc2cc(O)cc(-c3nc4c5c(nc(OC[C@@]67CCCN6C/C(=C\F)C7)nc5c3F)N3CCCC[C@H]3CO4)c12] |
| D | 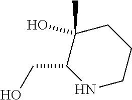 | DMF | 53 | 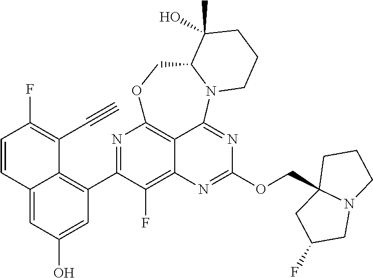 [SMILES: C#Cc1c(F)ccc2cc(O)cc(-c3nc4c5c(nc(OC[C@@]67CCCN6C[C@H](F)C7)nc5c3F)N3CCC[C@@](C)(O)[C@H]3CO4)c12] |
| D | 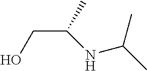 | DMF | 56 | 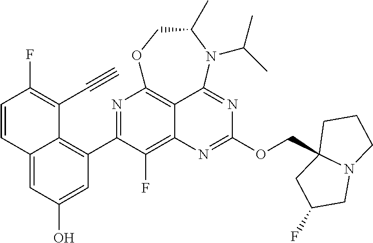 [SMILES: C#Cc1c(F)ccc2cc(O)cc(-c3nc4c5c(nc(OC[C@@]67CCCN6C[C@H](F)C7)nc5c3F)N(C(C)C)[C@@H](C)CO4)c12] |
| D | 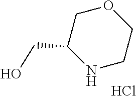 | THF | 58 | 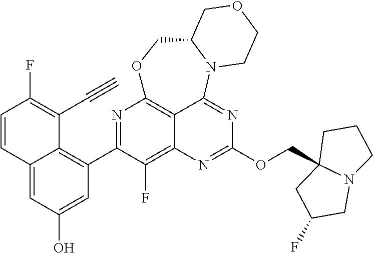 [SMILES: C#Cc1c(F)ccc2cc(O)cc(-c3nc4c5c(nc(OC[C@@]67CCCN6C[C@H](F)C7)nc5c3F)N3CCOC[C@H]3CO4)c12] |
| D | 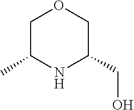 | DMF | 59 | 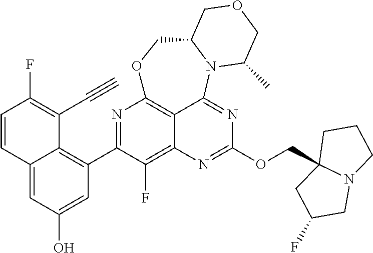 [SMILES: C#Cc1c(F)ccc2cc(O)cc(-c3nc4c5c(nc(OC[C@@]67CCCN6C[C@H](F)C7)nc5c3F)N3[C@@H](COC[C@@H]3C)CO4)c12] |
| E | 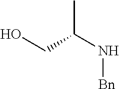 | THF | 74 | 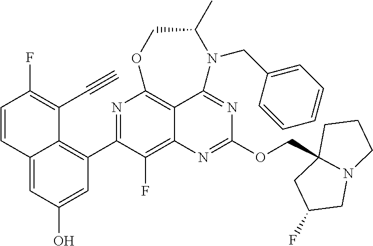 [SMILES: C#Cc1c(F)ccc2cc(O)cc(-c3nc4c5c(nc(OC[C@@]67CCCN6C[C@H](F)C7)nc5c3F)N(Cc3ccccc3)[C@@H](C)CO4)c12] |
| D | 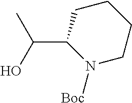 [SMILES: CC(O)[C@@H]1CCCCN1C(=O)OC(C)(C)C] | DMF | 75 | 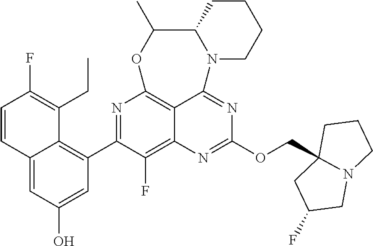 [SMILES: CCc1c(F)ccc2cc(O)cc(-c3nc4c5c(nc(OC[C@@]67CCCN6C[C@H](F)C7)nc5c3F)N3CCCC[C@H]3C(C)O4)c12] |
| D | 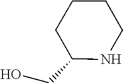 | DMF | 76 | 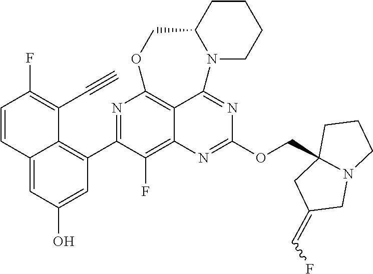 [SMILES: C#Cc1c(F)ccc2cc(O)cc(-c3nc4c5c(nc(OC[C@@]67CCCN6CC(=CF)C7)nc5c3F)N3CCCC[C@H]3CO4)c12] |
| E | 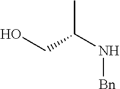 | THF | 77 | 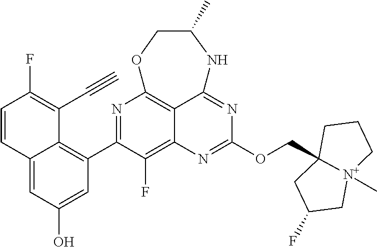 [SMILES: C#Cc1c(F)ccc2cc(O)cc(-c3nc4c5c(nc(OC[C@@]67CCC[N+]6(C)C[C@H](F)C7)nc5c3F)N[C@@H](C)CO4)c12] |
| D | 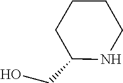 | DMF | 78 | 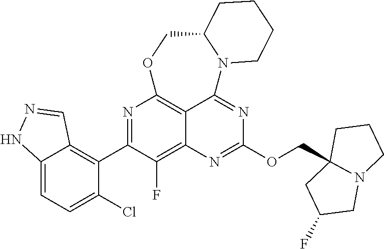 [SMILES: Fc1c(-c2c(Cl)ccc3[nH]ncc23)nc2c3c(nc(OC[C@@]45CCCN4C[C@H](F)C5)nc13)N1CCCC[C@H]1CO2] |
| D | 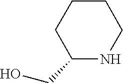 | DMF | 79 | 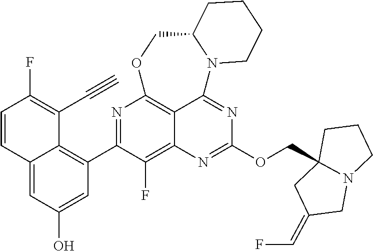 [SMILES: C#Cc1c(F)ccc2cc(O)cc(-c3nc4c5c(nc(OC[C@@]67CCCN6C/C(=C/F)C7)nc5c3F)N3CCCC[C@H]3CO4)c12] |
| D | 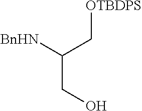 | THF | 80 | 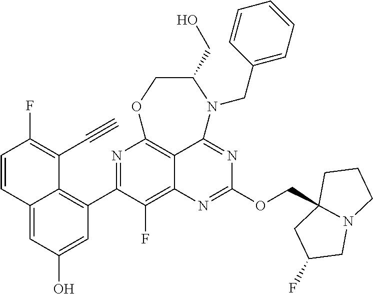 [SMILES: C#Cc1c(F)ccc2cc(O)cc(-c3nc4c5c(nc(OC[C@@]67CCCN6C[C@H](F)C7)nc5c3F)N(Cc3ccccc3)[C@@H](CO)CO4)c12] |
| D | 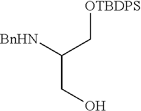 | THF | 81 | 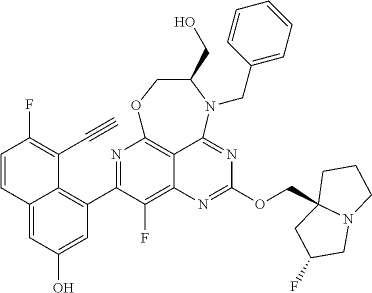 [SMILES: C#Cc1c(F)ccc2cc(O)cc(-c3nc4c5c(nc(OC[C@@]67CCCN6C[C@H](F)C7)nc5c3F)N(Cc3ccccc3)[C@H](CO)CO4)c12] |
| E | 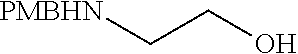 | THF | 82 | 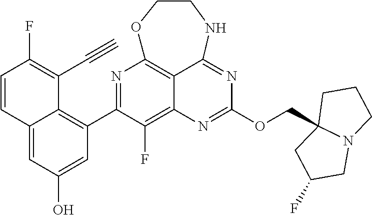 [SMILES: C#Cc1c(F)ccc2cc(O)cc(-c3nc4c5c(nc(OC[C@@]67CCCN6C[C@H](F)C7)nc5c3F)NCCO4)c12] |
| D | 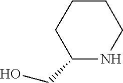 | DMF | 83 | 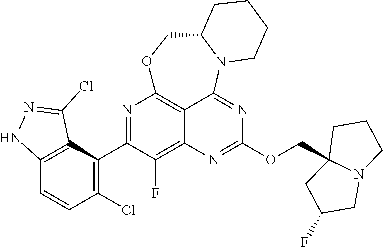 |
| D | 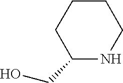 | DMF | 84 | 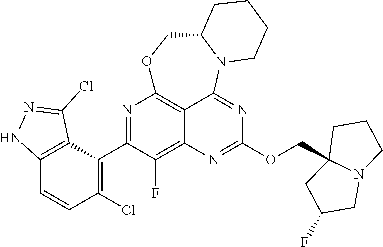 |
| D | 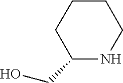 | DMF | 85 | 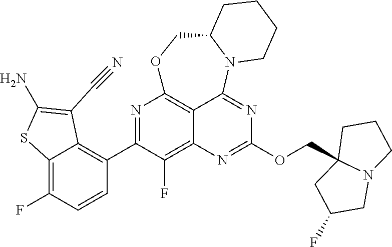 [SMILES: N#Cc1c(N)sc2c(F)ccc(-c3nc4c5c(nc(OC[C@@]67CCCN6C[C@H](F)C7)nc5c3F)N3CCCC[C@H]3CO4)c12] |
| D | 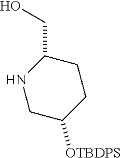 | DMF | 86 | 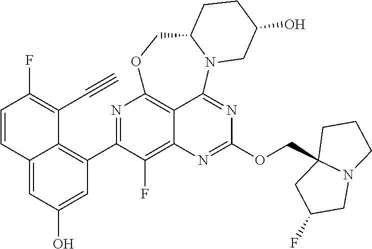 [SMILES: C#Cc1c(F)ccc2cc(O)cc(-c3nc4c5c(nc(OC[C@@]67CCCN6C[C@H](F)C7)nc5c3F)N3C[C@@H](O)CC[C@H]3CO4)c12] |
| D | 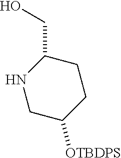 | DMF | 87 | 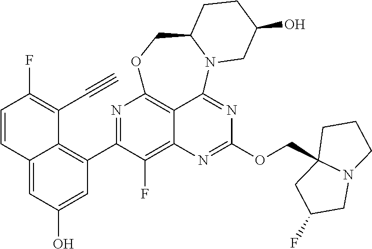 [SMILES: C#Cc1c(F)ccc2cc(O)cc(-c3nc4c5c(nc(OC[C@@]67CCCN6C[C@H](F)C7)nc5c3F)N3C[C@H](O)CC[C@@H]3CO4)c12] |
| A | 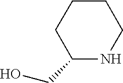 | DMF | 88 | 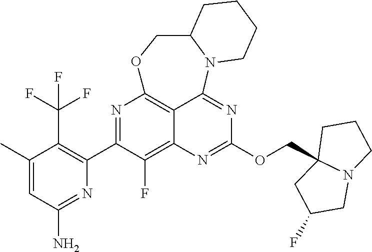 [SMILES: Cc1cc(N)nc(-c2nc3c4c(nc(OC[C@@]56CCCN5C[C@H](F)C6)nc4c2F)N2CCCCC2CO3)c1C(F)(F)F] |
| A | 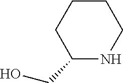 | DMF | 89 | 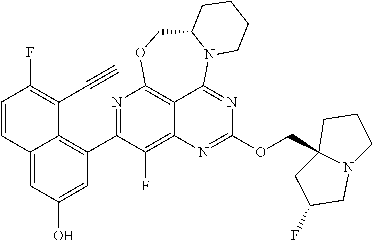 [SMILES: C#Cc1c(F)ccc2cc(O)cc(-c3nc4c5c(nc(OC[C@@]67CCCN6C[C@H](F)C7)nc5c3F)N3CCCC[C@H]3CO4)c12] |
| E | 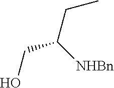 | THF | 90 | 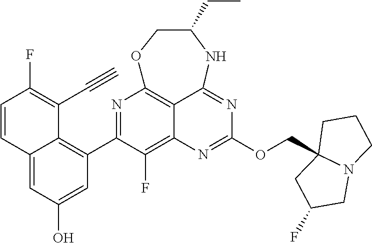 [SMILES: C#Cc1c(F)ccc2cc(O)cc(-c3nc4c5c(nc(OC[C@@]67CCCN6C[C@H](F)C7)nc5c3F)N[C@@H](CC)CO4)c12] |
| D | 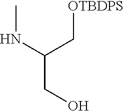 | THF | 91 | 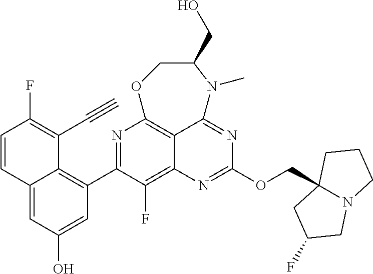 [SMILES: C#Cc1c(F)ccc2cc(O)cc(-c3nc4c5c(nc(OC[C@@]67CCCN6C[C@H](F)C7)nc5c3F)N(C)[C@H](CO)CO4)c12] |
| D | 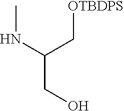 | THF | 92 | 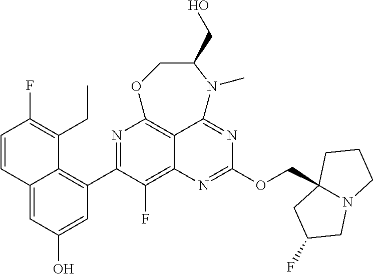 [SMILES: CCc1c(F)ccc2cc(O)cc(-c3nc4c5c(nc(OC[C@@]67CCCN6C[C@H](F)C7)nc5c3F)N(C)[C@H](CO)CO4)c12] |
| D | 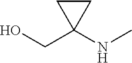 | DMF | 93 | 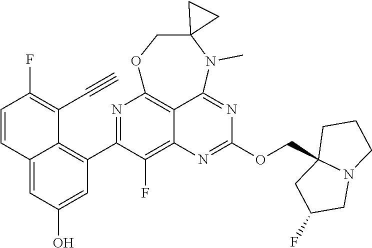 [SMILES: C#Cc1c(F)ccc2cc(O)cc(-c3nc4c5c(nc(OC[C@@]67CCCN6C[C@H](F)C7)nc5c3F)N(C)C3(CC3)CO4)c12] |
| D | 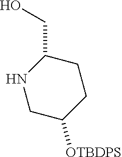 | DMF | 94 | 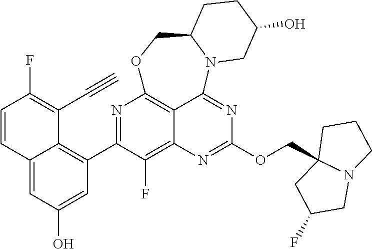 [SMILES: C#Cc1c(F)ccc2cc(O)cc(-c3nc4c5c(nc(OC[C@@]67CCCN6C[C@H](F)C7)nc5c3F)N3C[C@@H](O)CC[C@@H]3CO4)c12] |
| E | 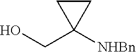 | THF | 95 | 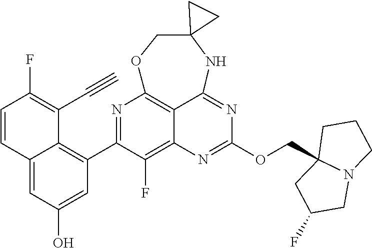 [SMILES: C#Cc1c(F)ccc2cc(O)cc(-c3nc4c5c(nc(OC[C@@]67CCCN6C[C@H](F)C7)nc5c3F)NC3(CC3)CO4)c12] |
| F, D | 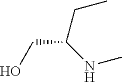 | THF | 96 | 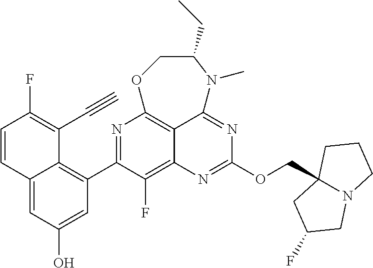 [SMILES: C#Cc1c(F)ccc2cc(O)cc(-c3nc4c5c(nc(OC[C@@]67CCCN6C[C@H](F)C7)nc5c3F)N(C)[C@@H](CC)CO4)c12] |
| D | 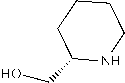 | DMF | 97 | 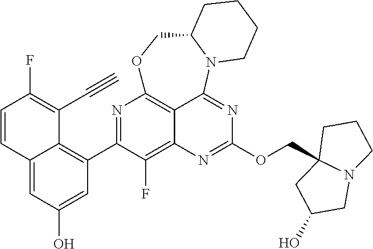 [SMILES: C#Cc1c(F)ccc2cc(O)cc(-c3nc4c5c(nc(OC[C@@]67CCCN6C[C@H](O)C7)nc5c3F)N3CCCC[C@H]3CO4)c12] |
| D | 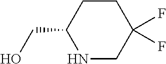 | THF | 98 | 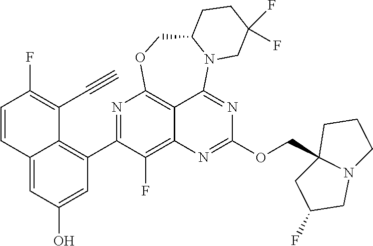 [SMILES: C#Cc1c(F)ccc2cc(O)cc(-c3nc4c5c(nc(OC[C@@]67CCCN6C[C@H](F)C7)nc5c3F)N3CC(F)(F)CC[C@H]3CO4)c12] |
| F, D | 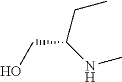 | THF | 99 | 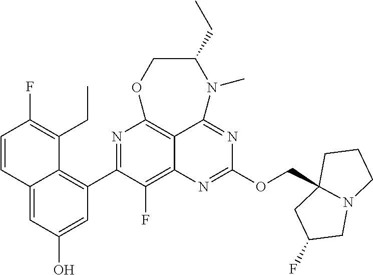 [SMILES: CCc1c(F)ccc2cc(O)cc(-c3nc4c5c(nc(OC[C@@]67CCCN6C[C@H](F)C7)nc5c3F)N(C)[C@@H](CC)CO4)c12] |
| D | 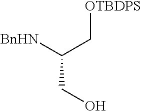 | THF | 100 | 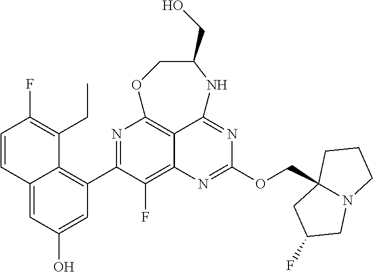 [SMILES: CCc1c(F)ccc2cc(O)cc(-c3nc4c5c(nc(OC[C@@]67CCCN6C[C@H](F)C7)nc5c3F)N[C@H](CO)CO4)c12] |
| D | 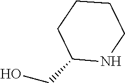 | DMF | 101 | 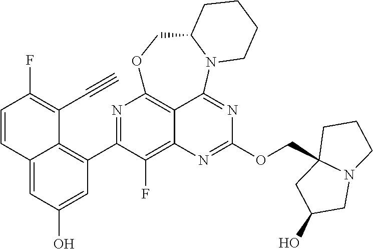 [SMILES: C#Cc1c(F)ccc2cc(O)cc(-c3nc4c5c(nc(OC[C@@]67CCCN6C[C@@H](O)C7)nc5c3F)N3CCCC[C@H]3CO4)c12] |
| E | 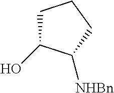 | DMF | 102 | 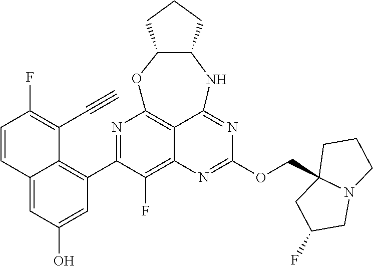 [SMILES: C#Cc1c(F)ccc2cc(O)cc(-c3nc4c5c(nc(OC[C@@]67CCCN6C[C@H](F)C7)nc5c3F)N[C@H]3CCC[C@H]3O4)c12] |
| D | 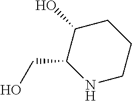 | DMF | 103 | 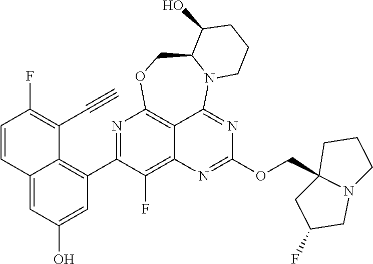 [SMILES: C#Cc1c(F)ccc2cc(O)cc(-c3nc4c5c(nc(OC[C@@]67CCCN6C[C@H](F)C7)nc5c3F)N3CCC[C@H](O)[C@@H]3CO4)c12] |
| D | 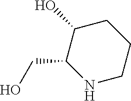 | DMF | 104 | 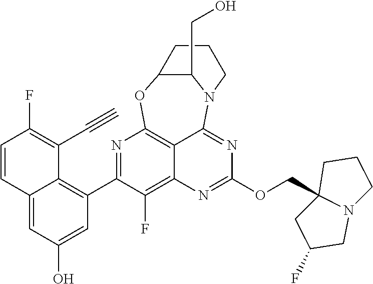 [SMILES: C#Cc1c(F)ccc2cc(O)cc(-c3nc4c5c(nc(OC[C@@]67CCCN6C[C@H](F)C7)nc5c3F)N3CCCC(O4)C3CO)c12] |
| D | 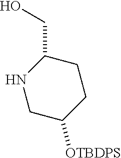 | DMF | 105 | 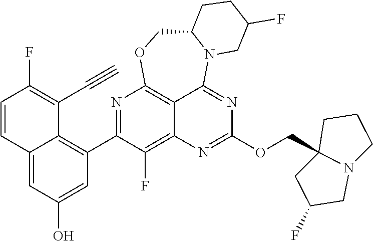 [SMILES: C#Cc1c(F)ccc2cc(O)cc(-c3nc4c5c(nc(OC[C@@]67CCCN6C[C@H](F)C7)nc5c3F)N3CC(F)CC[C@H]3CO4)c12] |
| D | 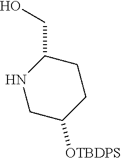 | DMF | 106 | 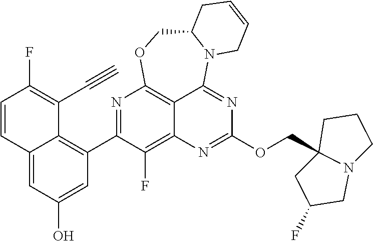 [SMILES: C#Cc1c(F)ccc2cc(O)cc(-c3nc4c5c(nc(OC[C@@]67CCCN6C[C@H](F)C7)nc5c3F)N3CC=CC[C@H]3CO4)c12] |
| D | 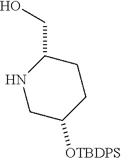 | DMF | 107 | 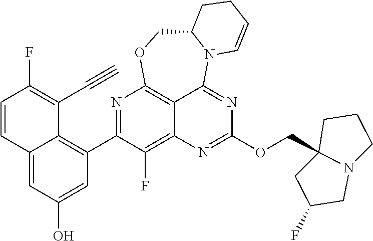 [SMILES: C#Cc1c(F)ccc2cc(O)cc(-c3nc4c5c(nc(OC[C@@]67CCCN6C[C@H](F)C7)nc5c3F)N3C=CCC[C@H]3CO4)c12] |
| F, E | 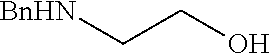 | THF | 108 | 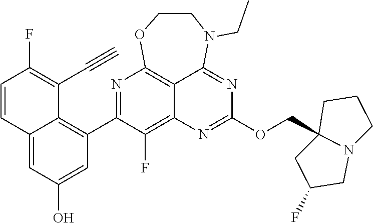 [SMILES: C#Cc1c(F)ccc2cc(O)cc(-c3nc4c5c(nc(OC[C@@]67CCCN6C[C@H](F)C7)nc5c3F)N(CC)CCO4)c12] |
| D | 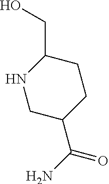 [SMILES: NC(=O)C1CCC(CO)NC1] | DMF | 109 | 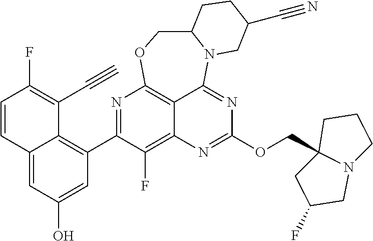 [SMILES: C#Cc1c(F)ccc2cc(O)cc(-c3nc4c5c(nc(OC[C@@]67CCCN6C[C@H](F)C7)nc5c3F)N3CC(C#N)CCC3CO4)c12] |
| D | 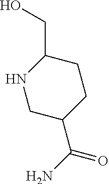 [SMILES: NC(=O)C1CCC(CO)NC1] | DMF | 110 | 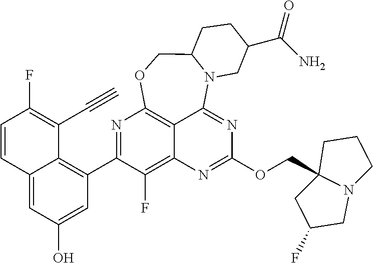 [SMILES: C#Cc1c(F)ccc2cc(O)cc(-c3nc4c5c(nc(OC[C@@]67CCCN6C[C@H](F)C7)nc5c3F)N3CC(C(N)=O)CCC3CO4)c12] |
| F, D | 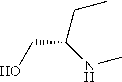 | THF | 111 | 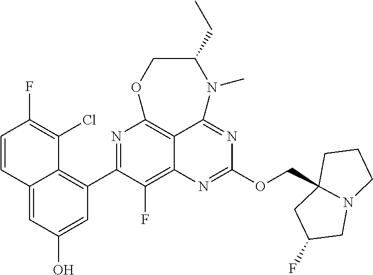 [SMILES: CC[C@H]1COc2nc(-c3cc(O)cc4ccc(F)c(Cl)c34)c(F)c3nc(OC[C@@]45CCCN4C[C@H](F)C5)nc(c23)N1C] |
| G, F, D | 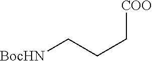 | — | 112 | 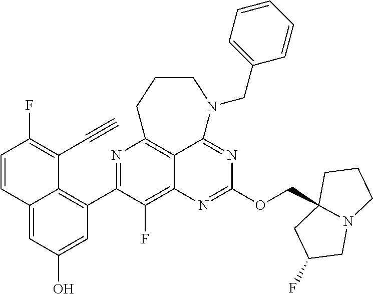 [SMILES: C#Cc1c(F)ccc2cc(O)cc(-c3nc4c5c(nc(OC[C@@]67CCCN6C[C@H](F)C7)nc5c3F)N(Cc3ccccc3)CCC4)c12] |
| D | 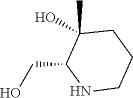 | DMF | 113 | 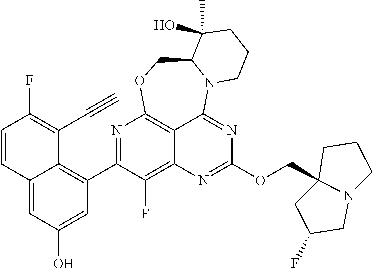 [SMILES: C#Cc1c(F)ccc2cc(O)cc(-c3nc4c5c(nc(OC[C@@]67CCCN6C[C@H](F)C7)nc5c3F)N3CCC[C@](C)(O)[C@@H]3CO4)c12] |
| D | 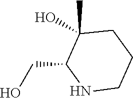 | DMF | 114 | 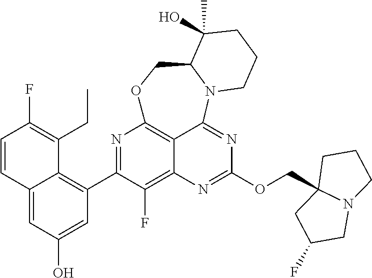 [SMILES: CCc1c(F)ccc2cc(O)cc(-c3nc4c5c(nc(OC[C@@]67CCCN6C[C@H](F)C7)nc5c3F)N3CCC[C@](C)(O)[C@@H]3CO4)c12] |
| D | 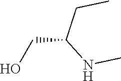 | THF | 115 | 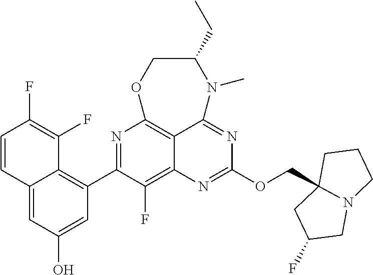 [SMILES: CC[C@H]1COc2nc(-c3cc(O)cc4ccc(F)c(F)c34)c(F)c3nc(OC[C@@]45CCCN4C[C@H](F)C5)nc(c23)N1C] |
| D | 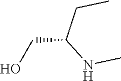 | DMF | 116 | 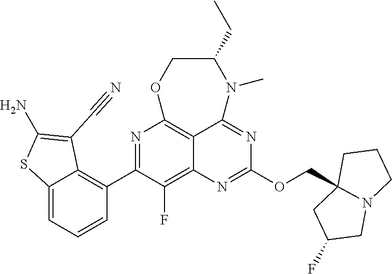 [SMILES: CC[C@H]1COc2nc(-c3cccc4sc(N)c(C#N)c34)c(F)c3nc(OC[C@@]45CCCN4C[C@H](F)C5)nc(c23)N1C] |
| F, D | 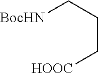 [SMILES: CC(C)(C)OC(=O)NCCCC(=O)O] | — | 117 | 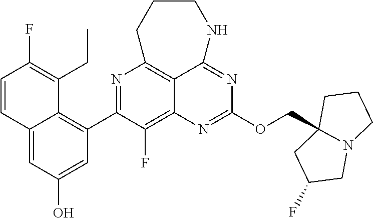 [SMILES: CCc1c(F)ccc2cc(O)cc(-c3nc4c5c(nc(OC[C@@]67CCCN6C[C@H](F)C7)nc5c3F)NCCC4)c12] |
| D | 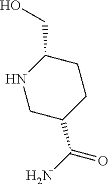 | DMF | 118 | 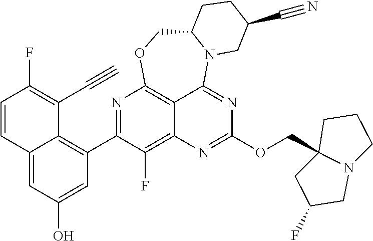 [SMILES: C#Cc1c(F)ccc2cc(O)cc(-c3nc4c5c(nc(OC[C@@]67CCCN6C[C@H](F)C7)nc5c3F)N3C[C@H](C#N)CC[C@H]3CO4)c12] |
| D | 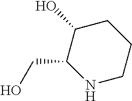 | THF | 119 | 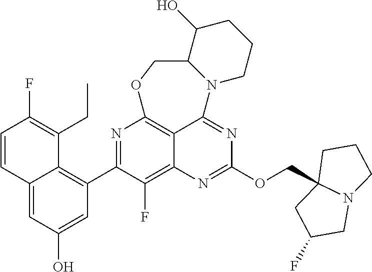 [SMILES: CCc1c(F)ccc2cc(O)cc(-c3nc4c5c(nc(OC[C@@]67CCCN6C[C@H](F)C7)nc5c3F)N3CCCC(O)C3CO4)c12] |
| D | 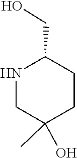 | DMF | 120 | 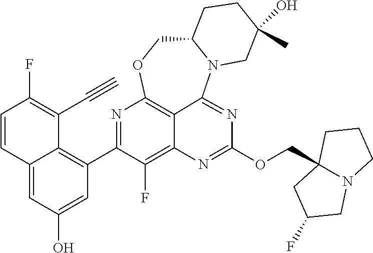 [SMILES: C#Cc1c(F)ccc2cc(O)cc(-c3nc4c5c(nc(OC[C@@]67CCCN6C[C@H](F)C7)nc5c3F)N3C[C@@](C)(O)CC[C@H]3CO4)c12] |
| H, D | 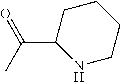 | — | 121 | 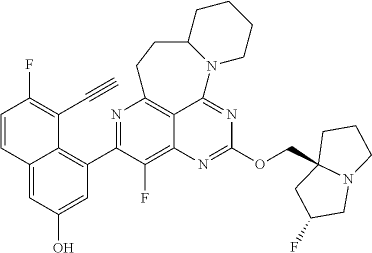 [SMILES: C#Cc1c(F)ccc2cc(O)cc(-c3nc4c5c(nc(OC[C@@]67CCCN6C[C@H](F)C7)nc5c3F)N3CCCCC3CC4)c12] |
| G, D | 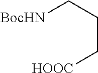 [SMILES: CC(C)(C)OC(=O)NCCCC(=O)O] | — | 122 | 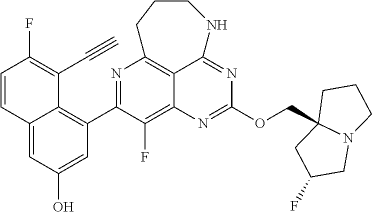 [SMILES: C#Cc1c(F)ccc2cc(O)cc(-c3nc4c5c(nc(OC[C@@]67CCCN6C[C@H](F)C7)nc5c3F)NCCC4)c12] |
| D | 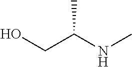 | THF | 123 | 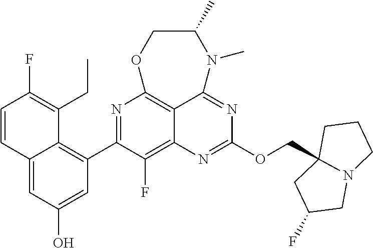 [SMILES: CCc1c(F)ccc2cc(O)cc(-c3nc4c5c(nc(OC[C@@]67CCCN6C[C@H](F)C7)nc5c3F)N(C)[C@@H](C)CO4)c12] |
| D | 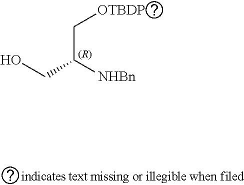 | THF | 124 | 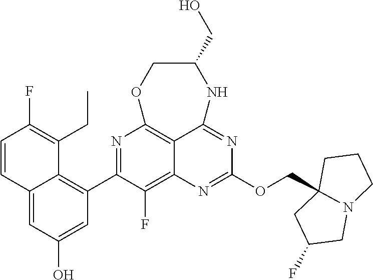 [SMILES: CCc1c(F)ccc2cc(O)cc(-c3nc4c5c(nc(OC[C@@]67CCCN6C[C@H](F)C7)nc5c3F)N[C@@H](CO)CO4)c12] |
| D | 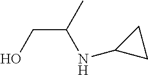 | THF | 125 | 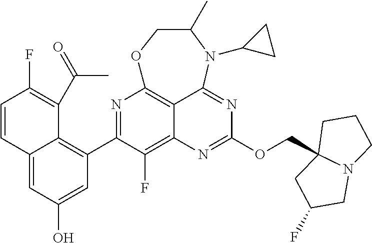 [SMILES: CC(=O)c1c(F)ccc2cc(O)cc(-c3nc4c5c(nc(OC[C@@]67CCCN6C[C@H](F)C7)nc5c3F)N(C3CC3)C(C)CO4)c12] |
| E | 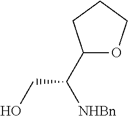 | THF | 126 | 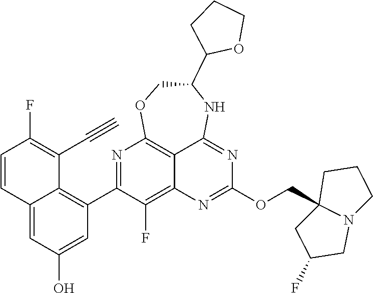 [SMILES: C#Cc1c(F)ccc2cc(O)cc(-c3nc4c5c(nc(OC[C@@]67CCCN6C[C@H](F)C7)nc5c3F)N[C@@H](C3CCCO3)CO4)c12] |
| F | 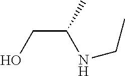 | THF | 127 | 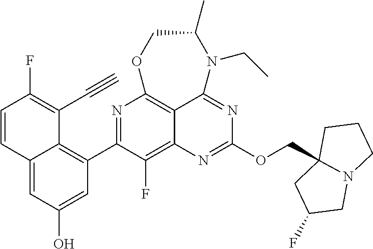 [SMILES: C#Cc1c(F)ccc2cc(O)cc(-c3nc4c5c(nc(OC[C@@]67CCCN6C[C@H](F)C7)nc5c3F)N(CC)[C@@H](C)CO4)c12] |
| D | 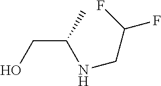 | THF | 128 | 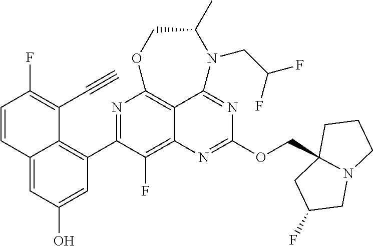 [SMILES: C#Cc1c(F)ccc2cc(O)cc(-c3nc4c5c(nc(OC[C@@]67CCCN6C[C@H](F)C7)nc5c3F)N(CC(F)F)[C@@H](C)CO4)c12] |
| D | 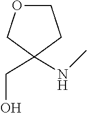 | DMF | 129 | 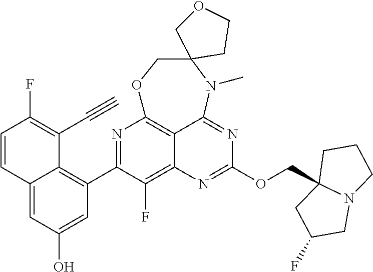 [SMILES: C#Cc1c(F)ccc2cc(O)cc(-c3nc4c5c(nc(OC[C@@]67CCCN6C[C@H](F)C7)nc5c3F)N(C)C3(CCOC3)CO4)c12] |
| D | 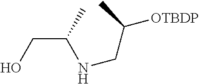 | THF | 130 | 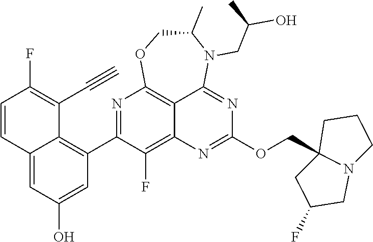 [SMILES: C#Cc1c(F)ccc2cc(O)cc(-c3nc4c5c(nc(OC[C@@]67CCCN6C[C@H](F)C7)nc5c3F)N(C[C@@H](C)O)[C@@H](C)CO4)c12] |
| D | 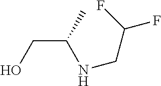 | THF | 131 | 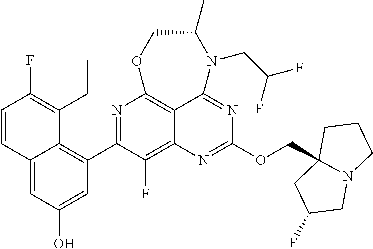 [SMILES: CCc1c(F)ccc2cc(O)cc(-c3nc4c5c(nc(OC[C@@]67CCCN6C[C@H](F)C7)nc5c3F)N(CC(F)F)[C@@H](C)CO4)c12] |
| D | 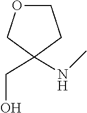 | DMF | 132 | 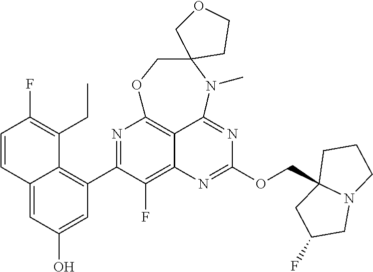 [SMILES: CCc1c(F)ccc2cc(O)cc(-c3nc4c5c(nc(OC[C@@]67CCCN6C[C@H](F)C7)nc5c3F)N(C)C3(CCOC3)CO4)c12] |
| D | 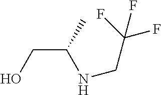 | DMF | 133 | 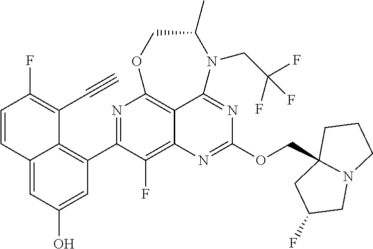 [SMILES: C#Cc1c(F)ccc2cc(O)cc(-c3nc4c5c(nc(OC[C@@]67CCCN6C[C@H](F)C7)nc5c3F)N(CC(F)(F)F)[C@@H](C)CO4)c12] |

### Preparation of (8aR,9R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol (Ex. 25) and (8aS,9S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol (Ex. 103)

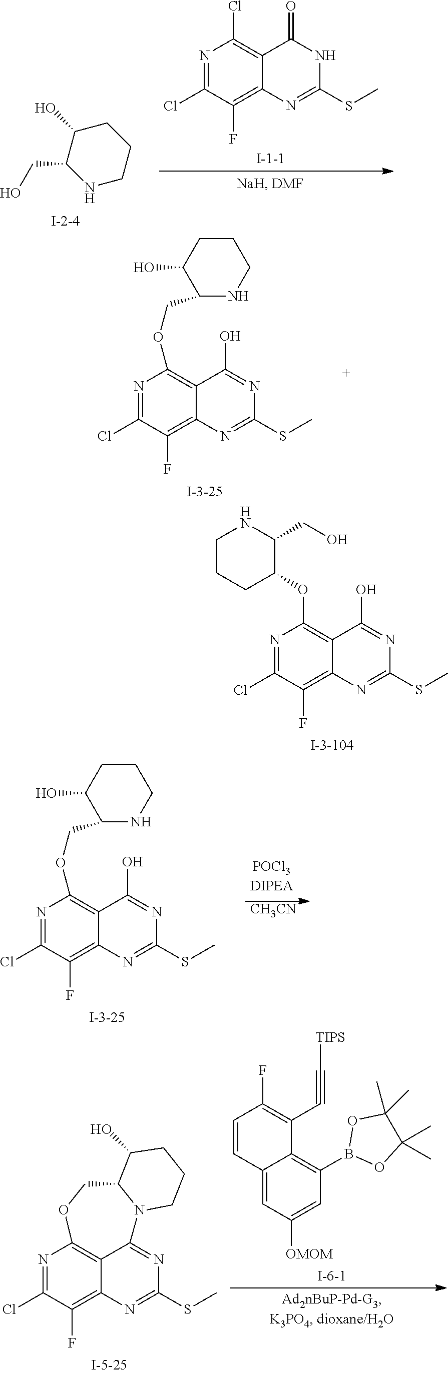

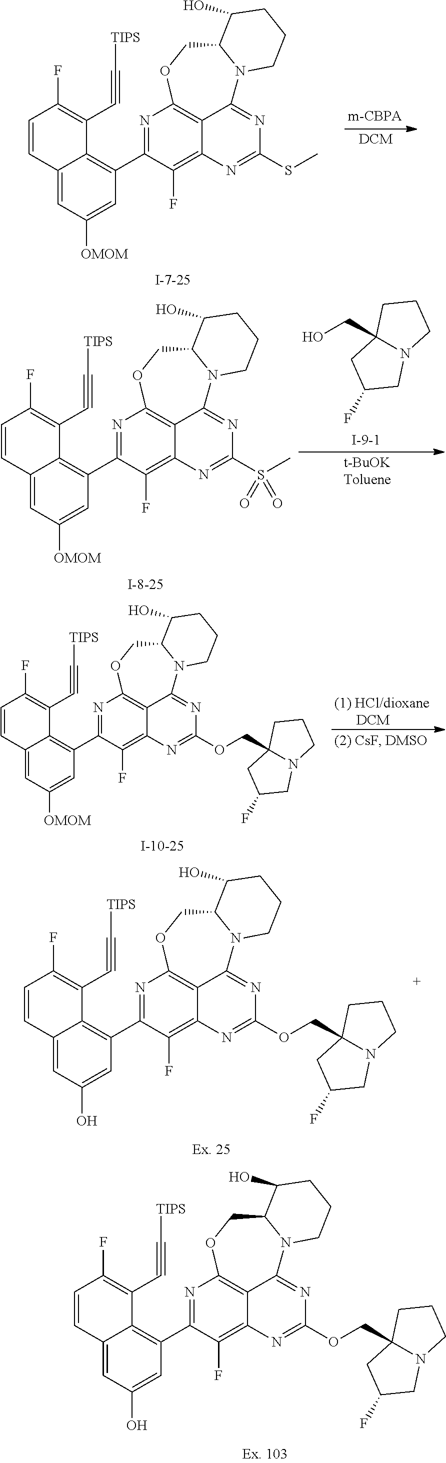

Step 1. To a solution of (2R,3R)-2-(hydroxymethyl)piperidin-3-ol (326 mg, 1.94 mmol, 1 eq, HCl) in DMF (7 mL) was added NaH (156 mg, 3.89 mmol, 60% purity, 2 eq) at 0° C. for 10 minutes. Then 5,7-dichloro-8-fluoro-2-methylsulfanyl-3H-pyrido[4,3-d]pyrimidin-4-one (436 mg, 1.56 mmol, 0.8 eq) was added to the mixture. The mixture was stirred at 25° C. for 16 h. On completion, the mixture was quenched with water (10 mL). The mixture was purified by reverse phase (Acetonitrile in H₂O from 0% to 100%) to give 7-chloro-8-fluoro-5-[[(2R,3R)-3-hydroxy-2-piperidyl]methoxy]-2-methylsulfanyl-pyrido[4,3-d]pyrimidin-4-ol (190 mg, 0.507 mmol, 26% yield) as a yellow solid (I-3-25) and 7-chloro-8-fluoro-5-[[(2R,3R)-2-(hydroxymethyl)-3-piperidyl]oxy]-2-methylsulfanyl-pyrido[4,3-d]pyrimidin-4-ol (90.0 mg, 0.24 mmol, 12% yield) as a yellow solid (I-3-104). Analytical data for I-3-25: ¹H NMR (400 MHz, DMSO-d₆) δ=9.46-8.62 (m, 1H), 5.69-5.48 (m, 1H), 4.80-4.58 (m, 1H), 4.40-4.24 (m, 1H), 4.10-3.95 (m, 1H), 3.58-3.48 (m, 1H), 3.22 (br d, J=12.4 Hz, 1H), 3.03-2.90 (m, 1H), 2.41-2.32 (m, 3H), 1.99-1.84 (m, 1H), 1.82-1.71 (m, 1H), 1.67-1.49 (m, 2H). Analytical data for I-3-104: ¹H NMR (400 MHz, DMSO-d6) δ=8.98-8.27 (m, 1H), 5.69-5.43 (m, 1H), 5.28-5.04 (m, 1H), 4.77-4.62 (m, 1H), 4.42-4.23 (m, 1H), 4.09-3.93 (m, 1H), 3.62-3.44 (m, 2H), 3.28-3.17 (m, 1H), 3.07-2.89 (m, 1H), 2.41-2.36 (m, 3H), 2.31-2.21 (m, 1H), 1.92 (d, J=12.0 Hz, 4H).

Step 2. To a solution of 7-chloro-8-fluoro-5-[[(2R,3R)-3-hydroxy-2-piperidyl]methoxy]-2-methylsulfanyl-pyrido[4,3-d]pyrimidin-4-ol (150 mg, 0.4 mmol, 1 eq, I-3-25) in Acetonitrile (3 mL) was added DIPEA (465 mg, 3.60 mmol, 9 eq) and POCl₃ (184 mg, 1.20 mmol, 3 eq). The mixture was stirred at 25° C. for 2 h. On completion, the mixture was quenched by water (30 mL) and extracted with EtOAc (15 mL*3). The combined organic layer was dried over anhydrous Na₂SO₄, filtered and the filtrate was concentrated in vacuum to give (6R,7R)-12-chloro-13-fluoro-16-methylsulfanyl-9-oxa-2,11,15,17-tetrazatetracyclo[8.7.1.02,7.014,18]octadeca-1 (17),10 (18),11,13,15-pentaen-6-ol (100 mg, 0.28 mmol, 70% yield) as a brown solid. LCMS: (M+1: 357.0).

Step 3. To a solution of (6R,7R)-12-chloro-13-fluoro-16-methylsulfanyl-9-oxa-2,11,15,17-tetrazatetracyclo[8.7.1.02,7.014,18]octadeca-1 (17),10 (18),11,13,15-pentaen-6-ol (100 mg, 0.28 mmol, 1 eq), 2-[2-fluoro-6-(methoxymethoxy)-8-(4,4,5,5-tetramethyl-1,3,2-dioxaborolan-2-yl)-1-naphthyl]ethynyl-triisopropyl-silane (215 mg, 0.42 mmol, 1.5 eq) in dioxane (4 mL) and H₂O (0.8 mL) was added K₃PO₄ (178 mg, 0.841 mmol, 3 eq) and [2-(2-aminophenyl)phenyl]palladium(1+); bis(1-adamantyl)-butyl-phosphane; methanesulfonate (20.4 mg, 0.028 mmol, 0.1 eq). The mixture was stirred at 80° C. for 2 h under N₂. On completion, the mixture was dried over anhydrous Na₂SO₄, filtered and the filtrate was concentrated in vacuum to give crude. The residue was purified by combi flash (4 g silica gel column, EtOAc in PE from 0% to 100%) to give (6R, 7R)-13-fluoro-12-[7-fluoro-3-(methoxymethoxy)-8-(2-triisopropylsilylethynyl)-1-naphthyl]-16-methylsulfanyl-9-oxa-2,11,15,17-tetrazatetracyclo[8.7.1.02,7.014,18]octadeca-1 (17),10 (18),11,13,15-pentaen-6-ol (120 mg, 0.169 mmol, 60% yield) as yellow solid. LCMS: (M+1: 707.2).

Step 4. To a solution of (6R,7R)-13-fluoro-12-[7-fluoro-3-(methoxymethoxy)-8-(2-triisopropylsilylethynyl)-1-naphthyl]-16-methylsulfanyl-9-oxa-2,11,15,17-tetrazatetracyclo[8.7.1.02,7.014,18]octadeca-1 (17),10 (18),11,13,15-pentaen-6-ol (120 mg, 0.169 mmol, 1 eq) in DCM (2 mL) was added m-CPBA (86.1 mg, 0.424 mmol, 85% purity, 2.5 eq) at 0° C. and the mixture was stirred at 25° C. for 20 minutes. On completion, the mixture was diluted with water (30 mL) and extracted with DCM (10 mL*3). The combined organic layer was dried over anhydrous Na₂SO₄, filtered and the filtrate was concentrated in vacuum to give (6R,7R)-13-fluoro-12-[7-fluoro-3-(methoxymethoxy)-8-(2-triisopropylsilylethynyl)-1-naphthyl]-16-methylsulfonyl-9-oxa-2,11,15,17-tetrazatetracyclo[8.7.1.02,7.014,18]octadeca-1 (17),10 (18),11,13,15-pentaen-6-ol (125 mg, 0.169 mmol, 99% yield) as a yellow solid.

Step 5. To a solution of [(2R,8S)-2-fluoro-1,2,3,5,6,7-hexahydropyrrolizin-8-yl]methanol (80.8 mg, 0.507 mmol, 3 eq) in Toluene (3 mL) was added t-BuOK (56.9 mg, 0.507 mmol, 3 eq) at 0° C. for 20 minutes. Then the mixture was added (6R,7R)-13-fluoro-12-[7-fluoro-3-(methoxymethoxy)-8-(2-triisopropylsilylethynyl)-1-naphthyl]-16-methylsulfonyl-9-oxa-2,11,15,17-tetrazatetracyclo[8.7.1.02,7.014,18]octadeca-1 (17),10 (18),11,13,15-pentaen-6-ol (125 mg, 0.169 mmol, 1 eq). The mixture was stirred at 25° C. for 2 h. On completion, the mixture was diluted with water (20 mL) and extracted with DCM (10 mL*3). The combined organic layer was dried over anhydrous Na₂SO₄, filtered and filtrate was concentrated in vacuum to give (6R,7R)-13-fluoro-16-[[(2R,8S)-2-fluoro-1,2,3,5,6,7-hexahydropyrrolizin-8-yl]methoxy]-12-[7-fluoro-3-(methoxymethoxy)-8-(2-triisopropylsilylethynyl)-1-naphthyl]-9-oxa-2,11,15,17-tetrazatetracyclo [8.7.1.02,7.014,18]octadeca-1 (17),10 (18),11,13,15-pentaen-6-ol (88.0 mg, crude) as a yellow solid. LCMS: (M+1: 818.3)

Step 6. To a solution of (6R,7R)-13-fluoro-16-[[(2R,8S)-2-fluoro-1,2,3,5,6,7-hexahydropyrrolizin-8-yl]methoxy]-12-[7-fluoro-3-(methoxymethoxy)-8-(2-triisopropylsilylethynyl)-1-naphthyl]-9-oxa-2,11,15,17-tetrazatetracyclo[8.7.1.02, 7.014,18]octadeca-1 (17),10 (18),11,13,15-pentaen-6-ol (88.0 mg, 0.107 mmol, 1 eq) in DCM (1 mL) was added HCl/dioxane (4 M, 0.2 mL, 7.44 eq). The mixture was stirred at 25° C. for 20 minutes. On completion, the mixture was concentrated in vacuum to give (6R,7R)-13-fluoro-16-[[(2R,8S)-2-fluoro-1,2,3,5,6,7-hexahydropyrrolizin-8-yl]methoxy]-12-[7-fluoro-3-hydroxy-8-(2-triisopropylsilylethynyl)-1-naphthyl]-9-oxa-2,11,15,17-tetrazatetracyclo[8.7.1.02,7.014,18]octadeca-1 (17),10 (18),11,13,15-pentaen-6-ol (80.0 mg, crude) as a red solid. LCMS: (M+1: 774.3).

To a solution of (6R,7R)-13-fluoro-16-[[(2R,8S)-2-fluoro-1,2,3,5,6,7-hexahydropyrrolizin-8-yl]methoxy]-12-[7-fluoro-3-hydroxy-8-(2-triisopropylsilylethynyl)-1-naphthyl]-9-oxa-2,11,15,17-tetrazatetracyclo [8.7.1.02,7.014,18]octadeca-1 (17),10 (18),11,13,15-pentaen-6-ol (80.0 mg, 0.103 mmol, 1 eq) in DMSO (1 mL) was added CsF (47.1 mg, 0.31 mmol, 3 eq). The mixture was stirred at 25° C. for 1 h. On completion, the mixture was filtered, and the filtrate was concentrated in vacuum to give a crude residue, which was purified by Prep-HPLC (column: Waters xbridge 150*25 mm 10 um; mobile phase: [water(NH₄HCO₃)-Acetonitrile]; gradient: 30%-60% B over 10 min) to give (6R,7R)-12-(8-ethynyl-7-fluoro-3-hydroxy-1-naphthyl)-13-fluoro-9-oxa-2,11,15,17-tetrazatetracyclo[8.7.1.02, 7.0144,18]octadeca-1 (17),10 (18),11,13,15-pentaene-6,16-diol as an off-white solid as a mixture of diastereomers. The product was purified by SFC separation (column: REGIS (R,R)WHELK-01 (250 mm*25 mm, 10 um); mobile phase: [Heptane-(IPA:Acetonitrile=4:1) (0.1% NH3·H2O)]; B %: 30%, isocratic elution mode) to give (6R,7R)-12-(8-ethynyl-7-fluoro-3-hydroxy-1-naphthyl)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014.18]octadeca-1 (17),10 (18),11,13,15-pentaen-6-ol (10 mg, 0.016 mmol, 15.66% yield) as a white solid and (6S,7S)-12-(8-ethynyl-7-fluoro-3-hydroxy-1-naphthyl)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaen-6-ol (10 mg, 0.016 mmol, 15.66% yield) as a white solid. The crude products were purified by Prep-HPLC (column: Phenomenex luna C18 150*25 mm*10 um; mobile phase: [water(FA)-ACN]; gradient: 14%-44% B over 10 min) to give (6R,7R)-12-(8-ethynyl-7-fluoro-3-hydroxy-1-naphthyl)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.0^{2,7}0^{14,18}]octadeca-1 (17),10 (18),11,13,15-pentaen-6-ol (1.62 mg, 0.0026 mmol, 15.78% yield, 97.4% purity) as an off-white solid (Ex. 25) and (6S,7S)-12-(8-ethynyl-7-fluoro-3-hydroxy-1-naphthyl)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.0^{2,7}.0^{14,18}]octadeca-1 (17),10 (18),11,13,15-pentaen-6-ol (3.18 mg, 0.005 mmol, 30.97% yield, 97.4% purity) as off-white solid (Ex. 103). Analytical data for Ex. 25: ¹H NMR (400 MHz, MeOD-d₄) δ=7.92-7.79 (m, 1H), 7.40-7.29 (m, 2H), 7.18 (br d, J=5.6 Hz, 1H), 5.55-5.17 (m, 2H), 4.71-4.56 (m, 3H), 4.55-4.45 (m, 1H), 4.44-4.34 (m, 1H), 4.23-4.08 (m, 2H), 3.69-3.43 (m, 4H), 3.26-3.11 (m, 2H), 2.57-2.09 (m, 6H), 2.04-1.89 (m, 3H), 1.63 (s, 1H); LCMS m/z: 618.1 (M+1). Analytical data for Ex. 103: ¹H NMR (400 MHz, MeOD-d₄) δ=7.86 (dd, J=6.0, 8.8 Hz, 1H), 7.42-7.28 (m, 2H), 7.25-7.15 (m, 1H), 5.58-5.36 (m, 1H), 5.35-5.19 (m, 1H), 4.69-4.41 (m, 5H), 4.30-4.05 (m, 2H), 3.83-3.45 (m, 4H), 3.29-3.11 (m, 2H), 2.59-1.94 (m, 9H), 1.80-1.58 (m, 1H); LCMS m/z: 618.1 (M+1).

### Preparation of 5-ethyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-10-methyl-9,10-dihydro-8H-7-oxa-1,3,6,10-tetraazacyclohepta[1,2,3-de]naphthalen-5-yl]naphthalen-2-ol (Ex. 28) and 5-ethyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-10-methyl-9,10-dihydro-8H-7-oxa-1,3,6,10-tetraazacyclohepta[1,2,3-de]naphthalen-5-yl]naphthalen-2-ol (Ex. 92)

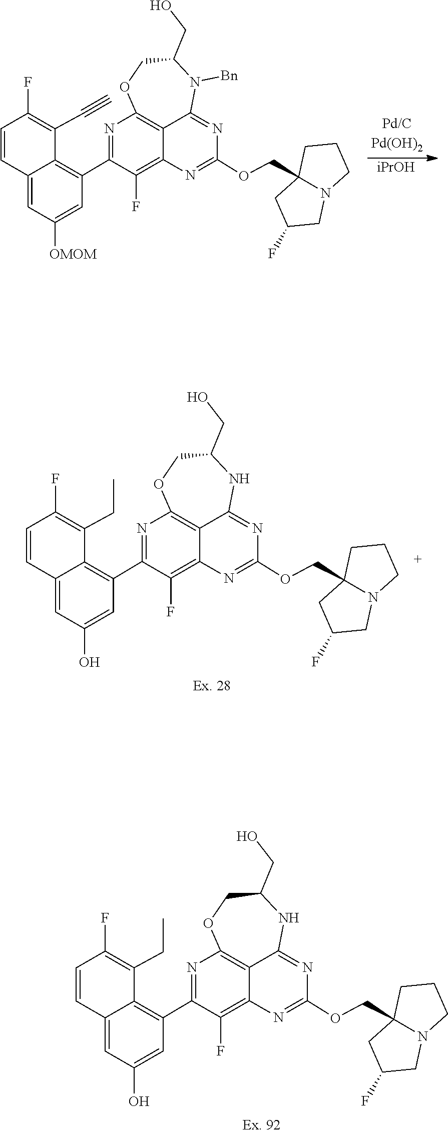

To a solution of 5-ethynyl-6-fluoro-4-[6-fluoro-3-[[(2R,8S)-2-fluoro-1,2,3,5,6,7-hexahydropyrrolizin-8-yl]methoxy]-12-(hydroxymethyl)-13-methyl-10-oxa-2,4,8,13-tetrazatricyclo[7.4.1.0^{5,14}]tetradeca-1,3,5,7,9 (14)-pentaen-7-yl]naphthalen-2-ol (100 mg, 0.169 mmol, 1 eq, from the synthesis of Ex. 27) in i-PrOH (2 mL) was added Pd/C (25 mg, 0.235 mmol, 10% purity, 1.39 eq) and Pd(OH)₂ (125 mg, 0.178 mmol, 20% purity, 1.05 eq). The mixture was stirred at 25° C. for 16 h. On completion, the mixture was filtered and concentrated to give a residue to give 5-ethyl-6-fluoro-4-[6-fluoro-3-[[(2R,8S)-2-fluoro-1,2,3,5,6,7-hexahydropyrrolizin-8-yl]methoxy]-12-(hydroxymethyl)-13-methyl-10-oxa-2,4,8,13-tetrazatricyclo[7.4.1.0^{5,14}]tetradeca-1,3,5,7,9 (14)-pentaen-7-yl]naphthalen-2-ol (108 mg, crude) as a yellow solid. LCMS: (M+1: 596.3). The residue was purified by SFC (column: Chiralpak IE-3 50×4.6 mm I.D., 3 um; Mobile phase: Phase A for Hexane (0.05% IPAm), and Phase B for IPA+ACN (0.05% IPAm); Isocratic elution: B in A 25%) to give 5-ethyl-6-fluoro-4-[(12S)-6-fluoro-3-[[(2R,8S)-2-fluoro-1,2,3,5,6,7-hexahydropyrrolizin-8-yl]methoxy]-12-(hydroxymethyl)-13-methyl-10-oxa-2,4,8,13-tetrazatricyclo[7.4.1.0^{5,14}]tetradeca-1,3,5,7,9 (14)-pentaen-7-yl]naphthalen-2-ol (14.87 mg, 0.025 mmol, 13.77% yield) as a white solid (Ex. 28) and 5-ethyl-6-fluoro-4-[(12R)-6-fluoro-3-[[(2R,8S)-2-fluoro-1,2,3,5,6,7-hexahydropyrrolizin-8-yl]methoxy]-12-(hydroxymethyl)-13-methyl-10-oxa-2,4,8,13-tetrazatricyclo[7.4.1.0^{5,14}]tetradeca-1,3,5,7,9 (14)-pentaen-7-yl]naphthalen-2-ol (6.99 mg, 0.012 mmol, 6.47% yield) as a white solid (Ex. 92). Analytical data for Ex. 28: ¹H NMR (400 MHz, MeOH-d₄) δ=7.69-7.62 (m, 1H), 7.31-7.19 (m, 2H), 7.14-6.98 (m, 1H), 5.46-5.26 (m, 1H), 4.52-4.41 (m, 2H), 4.38-4.31 (m, 1H), 4.13-4.07 (m, 1H), 3.96-3.87 (m, 1H), 3.85-3.77 (m, 1H), 3.58-3.34 (m, 6H), 3.17-3.09 (m, 1H), 2.64-2.29 (m, 4H), 2.28-2.16 (m, 2H), 2.14-2.02 (m, 2H), 2.02-1.90 (m, 1H), 0.93-0.80 (m, 3H); LCMS: (M+1: 596.0). Analytical data for Ex. 92: ¹H NMR (400 MHz, MeOH-d₄) δ=7.70-7.61 (m, 1H), 7.31-7.20 (m, 2H), 7.14-6.99 (m, 1H), 5.52-5.34 (m, 1H), 4.58-4.42 (m, 3H), 4.15-4.07 (m, 1H), 3.96-3.87 (m, 1H), 3.87-3.76 (m, 1H), 3.73-3.47 (m, 6H), 3.28-3.19 (m, 1H), 2.67-2.33 (m, 4H), 2.32-2.22 (m, 2H), 2.21-2.10 (m, 2H), 2.09-1.97 (m, 1H), 0.93-0.80 (m, 3H); LCMS: (M+1: 596.1).

### Preparation of (8aS,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-11-ol (Ex. 29) and (8aR,11S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-11-ol (Ex. 94)

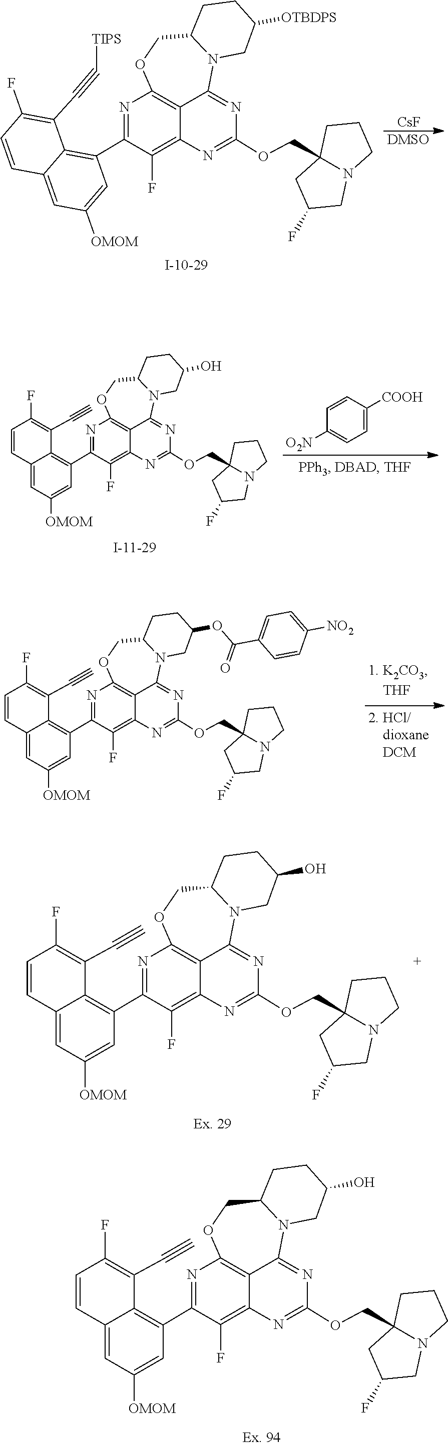

Step L. I-10-29 was prepared following General Method D (Step 1 to Step 5), using intermediate I-2-8 in Step 1. To a solution of I-10-29 (tert-butyl-[[(4S,7S)-13-fluoro-16-[[(2R,8S)-2-fluoro-1,2,3,5,6,7-hexahydropyrrolizin-8-yl]methoxy]-12-[7-fluoro-3-(methoxymethoxy)-8-(2-triisopropylsilylethynyl)-1-naphthyl]-9-oxa-2,11,15,17-tetrazatetracyclo [8.7.1.02,7.014,18]octadeca-1 (17),10 (18),11,13,15-pentaen-4-yl]oxy]-diphenyl-silane) (200 mg, 0.189 mmol, 1 eq, I-10-29) in DMSO (2 mL) was added CsF (287 mg, 1.89 mmol, 10 eq). The mixture was stirred at 50° C. for 6 hours. On completion, the mixture was concentrated in vacuo to give a residue. The residue was purified by prep-HPLC (column: Waters xbridge 150*25 mm 10 um; mobile phase: [water(NH₄HCO₃)-ACN]; gradient: 30%-60% B over 10 min) to give (7S)-12-[8-ethynyl-7-fluoro-3-(methoxymethoxy)-1-naphthyl]-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02, 7.014,18]octadeca-1 (17),10 (18),11,13,15-pentaen-4-ol (75.0 mg, 0.111 mmol, 59% yield) as yellow oil (I-11-29). LCMS: (M+1: 662.2).

Step 2. A mixture of (7S)-12-[8-ethynyl-7-fluoro-3-(methoxymethoxy)-1-naphthyl]-13-fluoro-16-[[(2R,8S)-2-fluoro-1,2,3,5,6,7-hexahydropyrrolizin-8-yl]methoxy]-9-oxa-2,11,15,17-tetrazatetracyclo [8.7.1.02,7.014,18]octadeca-1 (17),10 (18),11,13,15-pentaen-4-ol (72.0 mg, 0.108 mmol, 1 eq), 4-nitrobenzoic acid (21.8 mg, 0.130 mmol, 1.2 eq), DBAD (37.5 mg, 0.163 mmol, 1.5 eq), PPh₃ (42.8 mg, 0.136 mmol, 1.5 eq) in THF (2 mL) was degassed and purged with N₂ 3 times, and then the mixture was stirred at 25° C. for 2 hour under N₂ atmosphere. On completion, the mixture was concentrated in vacuo to give a residue. The residue was purified by prep-HPLC (column: Waters xbridge 150*25 mm 10 um; mobile phase: [water(NH₄HCO₃)-ACN]; gradient: 46%-76% B over 14 min) to give [12-[8-ethynyl-7-fluoro-3-(methoxymethoxy)-1-naphthyl]-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaen-4-yl]4-nitrobenzoate (50.0 mg, 0.057 mmol, 53% yield) as a white solid. LCMS: (M+1: 811.3).

Step 3. To a solution of [12-[8-ethynyl-7-fluoro-3-(methoxymethoxy)-1-naphthyl]-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaen-4-yl]4-nitrobenzoate (50.0 mg, 0.062 mmol, 1 eq) in THF (1 mL) was added K₂CO₃ (17.0 mg, 0.123 mmol, 2 eq). The mixture was stirred at 25° C. for 1 hour. On completion, the mixture was concentrated in vacuo to give 12-[8-ethynyl-7-fluoro-3-(methoxymethoxy)-1-naphthyl]-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02, 7.014,18]octadeca-1 (17),10 (18),11,13,15-pentaen-4-ol (40 mg, 98% yield) as a white solid. LCMS: (M+1: 662.2).

To a solution of 12-[8-ethynyl-7-fluoro-3-(methoxymethoxy)-1-naphthyl]-13-fluoro-16-∥(2R,8S)-2-fluoro-1,2,3,5,6,7-hexahydropyrrolizin-8-yl]methoxyl-9-oxa-2,11,15,17-tetrazatetracyclo[8.7.1.02,7.014,18]octadeca-1 (17),10 (18),11,13,15-pentaen-4-ol (40.0 mg, 0.060 mmol, 1 eq) in DCM (1 mL) was added HCl/dioxane (4 M, 0.5 mL). The mixture was stirred at 25° C. for 1 hour. On completion, the mixture was filtered to give a residue. The filtrate was purified by prep-HPLC (column: Waters xbridge 150*25 mm 10 um; mobile phase: [water(NH₄HCO₃)-ACN]; gradient: 23%-53% B over 10 min.) to give 12-(8-ethynyl-7-fluoro-3-hydroxy-1-naphthyl)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaen-4-ol (22.96 mg, 0.037 mmol, 61% yield, 99% purity, 20 mg for SFC) was obtained as an off-white solid. ¹H NMR (400 MHz, DMSO-d₆) δ=10.13 (s, 1H), 7.99-7.91 (m, 1H), 7.45 (t, J=8.8 Hz, 1H), 7.36 (d, J=2.4 Hz, 1H), 7.19-7.08 (m, 1H), 5.37-5.10 (m, 3H), 4.55-4.37 (m, 2H), 4.16-4.05 (m, 2H), 4.05-3.97 (m, 1H), 3.92-3.83 (m, 1H), 3.64-3.45 (m, 1H), 3.16-3.04 (m, 2H), 3.02 (s, 1H), 2.87-2.80 (m, 1H), 2.76 (t, J=11.6 Hz, 1H), 2.15-1.99 (m, 4H), 1.91-1.76 (m, 4H), 1.76-1.65 (m, 1H), 1.61-1.52 (m, 1H); LCMS: (M+1: 618.2).

The residue was separated by SFC (column: DAICEL CHIRALPAK IC (250 mm*50 mm, 10 um); mobile phase: [Hexane-IPA/ACN (0.1% IPAm)]; B %: 20%, isocratic elution mode) to give (4R,7S)-12-(8-ethynyl-7-fluoro-3-hydroxy-1-naphthyl)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.0^{2,7}.0^{14,18}]octadeca-1 (17),10 (18),11,13,15-pentaen-4-ol (4.9 mg, 0.0078 mmol, 24% yield, 99% purity, Ex. 29) as a yellow solid and (4S,7R)-12-(8-ethynyl-7-fluoro-3-hydroxy-1-naphthyl)-13-fluoro-16-[[(2R,8S)-2-fluoro-1,2,3,5,6,7-hexahydropyrrolizin-8-yl]methoxy]-9-oxa-2,11,15,17-tetrazatetracyclo [8.7.1.0^{2,7}.0^{14,18}]octadeca-1 (17),10 (18),11,13,15-pentaen-4-ol (6.18 mg, 0.010 mmol, 30.90% yield, 100% purity, Ex. 94) as a yellow solid. Analytical data for Ex. 29: ¹H NMR (400 MHz, DMSO-d₆) δ=10.13 (s, 1H), 7.99-7.92 (m, 1H), 7.45 (t, J=9.2 Hz, 1H), 7.36 (d, J=2.0 Hz, 1H), 7.18-7.10 (m, 1H), 5.38-5.09 (m, 3H), 4.56-4.38 (m, 2H), 4.16-4.05 (m, 2H), 4.04-3.97 (m, 1H), 3.94-3.83 (m, 1H), 3.64-3.46 (m, 1H), 3.10 (d, J=9.2 Hz, 2H), 3.02 (s, 1H), 2.88-2.81 (m, 1H), 2.76 (t, J=12.0 Hz, 1H), 2.13 (s, 1H), 2.10-2.08 (m, 1H), 2.06 (s, 1H), 2.01 (s, 1H), 1.87-1.82 (m, 2H), 1.81-1.76 (m, 2H), 1.73-1.66 (m, 1H), 1.56 (d, J=10.0 Hz, 1H); LCMS: (M+1: 618.1). Analytical data for Ex. 94: ¹H NMR (400 MHz, DMSO-d6) δ=10.13 (s, 1H), 8.00-7.91 (m, 1H), 7.46 (t, J=9.2 Hz, 1H), 7.37 (d, J=2.0 Hz, 1H), 7.14 (d, J=13.2 Hz, 1H), 5.45-4.96 (m, 3H), 4.57-4.37 (m, 2H), 4.18-3.98 (m, 3H), 3.93-3.84 (m, 1H), 3.64-3.45 (m, 1H), 3.13 (d, J=2.4 Hz, 2H), 3.08-3.00 (m, 1H), 2.92-2.82 (m, 1H), 2.76 (t, J=11.2 Hz, 1H), 2.15-2.07 (m, 2H), 2.06-1.97 (m, 2H), 1.86 (d, J=12.8 Hz, 2H), 1.83-1.76 (m, 2H), 1.75-1.65 (m, 1H), 1.57 (d, J=9.6 Hz, 1H); LCMS: (M+1: 618.1).

Ex. 30 and Ex. 120 were prepared following General method D, using I-2-9 in Step 1, and using XPhos Pd G4 and Cs₂CO₃ (instead of cataCXium© A Pd G3 and K₃PO₄) in Step 3.

### Preparation of 4-[(9S)-9-cyclopropyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethynyl-6-fluoronaphthalen-2-ol (Ex. 31)

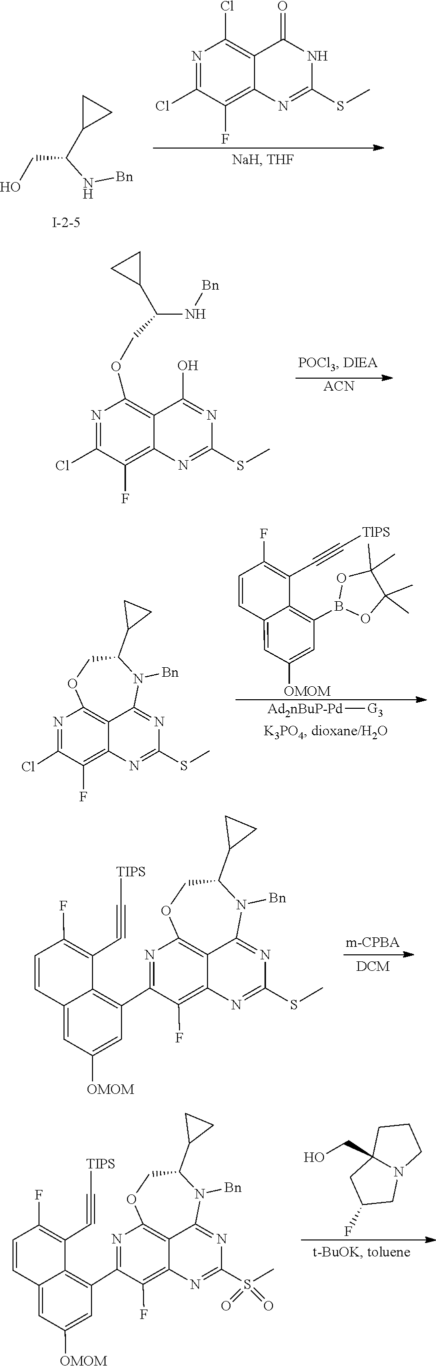

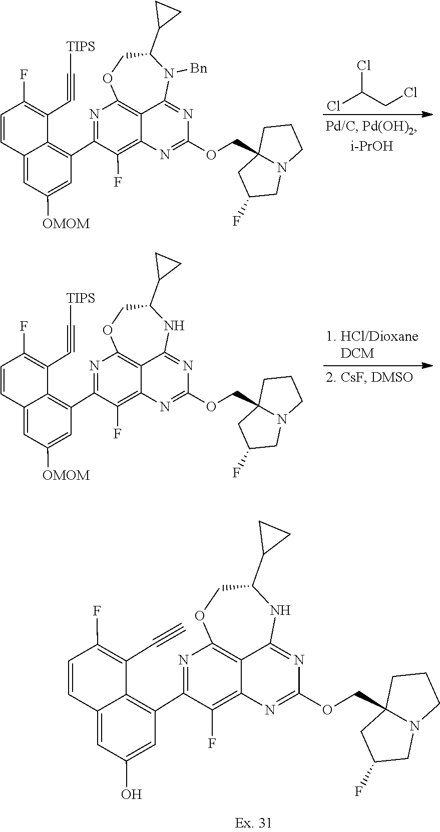

Step 1. To a solution of (2S)-2-(benzylamino)-2-cyclopropyl-ethanol (1.02 g, 5.36 mmol, 1.5 eq, Intermediate I-2-5) in THE (12 mL) was added NaH (285 mg, 7.14 mmol, 60% purity, 2 eq) at 0° C. for 0.5 h. Then 5, 7-dichloro-8-fluoro-2-methylsulfanyl-3H-pyrido [4,3-d]pyrimidin-4-one (1.00 g, 3.57 mmol, 1 eq) was added to the mixture and stirred at 25° C. for 2 h. On completion, the mixture was quenched with sat. NH₄Cl (30 mL) aqueous and extracted with EtOAc (20 mL*3). The combined organic layer was washed with brine (20 mL), dried over Na₂SO₄, filtered, and concentrated to give a residue. The residue was purified by column chromatography (SiO₂, Petroleum ether/THF=3:1 to 0:1) to give 5-[(2S)-2-(benzylamino)-2-cyclopropyl-ethoxy]-7-chloro-8-fluoro-2-methylsulfanyl-pyrido [4,3-d]pyrimidin-4-ol (1.50 g, 3.45 mmol, 96% yield) as yellow solid. ¹H NMR (400 MHz, DMSO-d₆) δ=7.48-7.14 (m, 5H), 4.44 (dd, J=3.6, 11.2 Hz, 1H), 4.31-4.16 (m, 2H), 4.13-4.05 (m, 1H), 3.01 (s, 1H), 2.34 (s, 2H), 2.30 (s, 3H), 0.85-0.73 (m, 1H), 0.55-0.43 (m, 1H), 0.40-0.29 (m, 1H), 0.20 (d, J=2.4 Hz, 2H); LCMS: (M+1: 435.1).

Step 2. To a solution of 5-[(2S)-2-(benzylamino)-2-cyclopropyl-ethoxy]-7-chloro-8-fluoro-2-methylsulfanyl-pyrido[4,3-d]pyrimidin-4-ol (1.45 g, 3.33 mmol, 1 eq) in Acetonitrile (15 mL) was added POCl₃ (1.02 g, 6.67 mmol, 0.621 mL, 2 eq) and DIPEA (2.59 g, 20.0 mmol, 6 eq). The mixture was stirred at 25° C. for 1 h. On completion, the mixture was concentrated in vacuum remove POCl₃. The residue was purified by column chromatography (SiO₂, Petroleum ether/Ethyl acetate=3:1 to 1:1) to give (12S)-13-benzyl-7-chloro-12-cyclopropyl-6-fluoro-3-methylsulfanyl-10-oxa-2,4,8,13-tetrazatricyclo[7.4.1.05,14]tetradeca-1,3,5,7,9 (14)-pentaene (1.10 g, 2.64 mmol, 79% yield) as yellow solid. ¹H NMR (400 MHz, DMSO-d6) δ=7.54-7.21 (m, 5H), 5.47 (br d, J=15.6 Hz, 1H), 4.87 (br d, J=15.6 Hz, 1H), 4.68 (br dd, J=4.0, 12.8 Hz, 1H), 4.48 (br d, J=12.8 Hz, 1H), 3.58-3.42 (m, 1H), 2.38-2.24 (m, 3H), 1.12-0.98 (m, 1H), 0.52 (br dd, J=5.6, 9.6 Hz, 3H), 0.43-0.32 (m, 1H). LCMS: EC8542-349-pla. (M+1: 417.0)

Step 3. A mixture of (12S)-13-benzyl-7-chloro-12-cyclopropyl-6-fluoro-3-methylsulfanyl-10-oxa-2,4,8,13-tetrazatricyclo[7.4.1.05,14]tetradeca-1,3,5,7,9 (14)-pentaene (300 mg, 0.719 mmol, 1 eq), 2-[2-fluoro-6-(methoxymethoxy)-8-(4,4,5,5-tetramethyl-1,3,2-dioxaborolan-2-yl)-1-naphthyl]ethynyl-triisopropyl-silane (553 mg, 1.08 mmol, 1.5 eq), [2-(2-aminophenyl)phenyl]palladium(1+); bis(1-adamantyl)-butyl-phosphane; methanesulfonate (52.4 mg, 0.072 mmol, 0.1 eq), K₃PO₄ (458 mg, 2.16 mmol, 3 eq) in H₂O (1 mL) and dioxane (5 mL) was degassed and purged with N₂ 3 times, and then the mixture was stirred at 80° C. for 2 h in microwave under N₂ atmosphere. On completion, the reaction mixture was diluted with water (20 mL) and was extracted with EtOAc (10 mL*3). The combined organic layer was washed with brine (20 mL), dried over Na₂SO₄, filtered, and concentrated to give a residue. The residue was purified by column chromatography (SiO₂, Petroleum ether/Ethyl acetate=3:1 to 1:1) to give 2-[8-[(12S)-13-benzyl-12-cyclopropyl-6-fluoro-3-methylsulfanyl-10-oxa-2,4,8,13-tetrazatricyclo[7.4.1.05,14]tetradeca-1,3,5,7,9 (14)-pentaen-7-yl]-2-fluoro-6-(methoxymethoxy)-1-naphthyl]ethynyl-triisopropyl-silane (510 mg, 0.664 mmol, 92% yield) as yellow solid. ¹H NMR (400 MHz, DMSO-d6) δ=8.14-8.06 (m, 1H), 7.76-7.70 (m, 1H), 7.57 (br t, J=8.8 Hz, 1H), 7.38-7.25 (m, 6H), 5.48 (s, 1H), 5.42-5.30 (m, 2H), 4.98 (s, 1H), 4.53 (s, 1H), 4.13-3.92 (m, 1H), 3.47-3.42 (m, 3H), 2.42-2.31 (m, 3H), 1.11-1.06 (m, 2H), 0.90-0.81 (m, 18H), 0.70-0.62 (m, 4H), 0.55 (br d, J=7.2 Hz, 2H), 0.42 (td, J=4.4, 8.8 Hz, 1H). LCMS: (M+1: 767.2).

Step 4. To a solution of 2-[8-[(12S)-13-benzyl-12-cyclopropyl-6-fluoro-3-methylsulfanyl-10-oxa-2,4,8,13-tetrazatricyclo[7.4.1.05,14]tetradeca-1,3,5,7,9 (14)-pentaen-7-yl]-2-fluoro-6-(methoxymethoxy)-1-naphthyl]ethynyl-triisopropyl-silane (500 mg, 0.651 mmol, 1 eq) in DCM (6 mL) was added m-CPBA (330 mg, 1.63 mmol, 85% purity, 2.5 eq). The mixture was stirred at 25° C. for 1 h. On completion, the reaction mixture was quenched by addition sat. Na₂SO₃ aqueous (20 mL) at 0° C., and then diluted with H₂O (20 mL) and extracted with DCM (10 mL*3). The combined organic layers were dried over Na₂SO₄, filtered and concentrated under reduced pressure to give 2-[8-[(12S)-13-benzyl-12-cyclopropyl-6-fluoro-3-methylsulfonyl-10-oxa-2,4,8,13-tetrazatricyclo tetradeca-1,3,5,7,9 (14)-pentaen-7-yl]-2-fluoro-6-(methoxymethoxy)-1-naphthyl]ethynyl-triisopropyl-silane (650 mg, crude) as yellow solid. LCMS: (M+1: 799.3).

Step 5. To a solution of [(2R,8S)-2-fluoro-1,2,3,5,6,7-hexahydropyrrolizin-8-yl]methanol (282 mg, 1.77 mmol, 3 eq) in toluene (10 mL) was added t-BuOK (198 mg, 1.77 mmol, 3 eq), the mixture was degassed and purged with N₂ 3 times for 30 min. Then 2-[8-[(12S)-13-benzyl-12-cyclopropyl-6-fluoro-3-methylsulfonyl-10-oxa-2,4,8,13-tetrazatricyclo[7.4.1.05,14]tetradeca-1,3,5,7,9 (14)-pentaen-7-yl]-2-fluoro-6-(methoxymethoxy)-1-naphthyl]ethynyl-triisopropyl-silane (590 mg, 0.590 mmol, 1 eq) was added and stirred at 25° C. for 1 h. On completion, the reaction mixture was diluted with water (20 mL) and was extracted with EtOAc (15 mL*3). The combined organic layer was washed with brine (20 mL), dried over Na₂SO₄, filtered, and concentrated to give a residue. The residue was purified by column chromatography (SiO₂, Petroleum ether/Ethyl acetate=3:1 to 0:1) to give 2-[8-[(12S)-13-benzyl-12-cyclopropyl-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05,14]tetradeca-1,3,5,7,9 (14)-pentaen-7-yl]-2-fluoro-6-(methoxymethoxy)-1-naphthyl]ethynyl-triisopropyl-silane (340 mg, 0.387 mmol, 65% yield) as yellow solid. LCMS: (M+1: 878.4).

Step 6. To a solution of 2-[8-[(12S)-13-benzyl-12-cyclopropyl-6-fluoro-3-[[(2R,8S)-2-fluoro-1,2,3,5,6,7-hexahydropyrrolizin-8-yl]methoxy]-10-oxa-2,4,8,13-tetrazatricyclo [7.4.1.05,14]tetradeca-1,3,5,7,9 (14)-pentaen-7-yl]-2-fluoro-6-(methoxymethoxy)-1-naphthyl]ethynyl-triisopropyl-silane (330 mg, 0.375 mmol, 1 eq) in i-PrOH (6 mL) was added Pd/C (50.0 mg, 0.047 mmol, 10% purity, 0.125 eq), Pd(OH)₂ (50.0 mg, 0.036 mmol, 10% purity, 0.1 eq) and 1,1,2-trichloroethane (100 mg, 0.751 mmol, 0.07 mL, 2 eq) was degassed and purged with H₂ (15 psi) 3 times, and then the mixture was stirred at 25° C. for 12 h under H₂ atmosphere. On completion, the mixture was filtered with diatomaceous earth. The residue was purified by column chromatography (SiO₂, DCM/MeOH=10:1 to 8:1) to give 2-[8-[(12S)-12-cyclopropyl-6-fluoro-3-[[(2R,8S)-2-fluoro-1,2,3,5,6,7-hexahydropyrrolizin-8-yl]methoxy]-10-oxa-2,4,8,13-tetrazatricyclo tetradeca-1,3,5,7,9 (14)-pentaen-7-yl]-2-fluoro-6-(methoxymethoxy)-1-naphthyl]ethynyl-triisopropyl-silane (50.0 mg, 0.0634 mmol, 16% yield) as yellow solid. LCMS: (M+1: 788.5).

Step 7. To a solution of 2-[8-[(12S)-12-cyclopropyl-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05,14]tetradeca-1,3,5,7,9 (14)-pentaen-7-yl]-2-fluoro-6-(methoxymethoxy)-1-naphthyl]ethynyl-triisopropyl-silane (45.0 mg, 0.057 mmol, 1 eq) in DCM (3 mL) was added HCl/dioxane (4 M, 0.60 mL, 42 eq). The mixture was stirred at 25° C. for 1 h. On completion, the mixture was concentrated in vacuum to give 4-[(12S)-12-cyclopropyl-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05,14]tetradeca-1,3,5,7,9 (14)-pentaen-7-yl]-6-fluoro-5-(2-triisopropylsilylethynyl)naphthalen-2-ol (50.0 mg, crude) as yellow solid. LCMS: (M+1: 744.3).

To a solution of 4-[(12S)-12-cyclopropyl-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05,14]tetradeca-1,3,5,7,9 (14)-pentaen-7-yl]-6-fluoro-5-(2-triisopropylsilylethynyl) naphthalen-2-ol (45.0 mg, 0.060 mmol, 1 eq) in DMSO (1 mL) was added CsF (45.9 mg, 0.302 mmol, 5 eq). The mixture was stirred at 25° C. for 30 min. On completion, the mixture was filtered and the filtrate was purified by Prep-HPLC purification (column: Phenomenex luna C18 150*25 mm*10 um; mobile phase: [water(FA)-ACN]; gradient: 14%-44% B over 11 min) to give 4-[(12S)-12-cyclopropyl-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05,14]tetradeca-1,3,5,7,9 (14)-pentaen-7-yl]-5-ethynyl-6-fluoro-naphthalen-2-ol (21.1 mg, 0.033 mmol, 54.3% yield, 98.5% purity, FA) as white solid (Ex. 31). ¹H NMR (400 MHz, DMSO-d₆) δ=10.14 (d, J=4.8 Hz, 1H), 9.41-9.25 (m, 1H), 7.96 (dd, J=6.0, 8.8 Hz, 1H), 7.53-7.43 (m, 1H), 7.37 (d, J=2.4 Hz, 1H), 7.16 (dd, J=2.4, 13.2 Hz, 1H), 5.40-5.18 (m, 1H), 4.62-4.50 (m, 1H), 4.43 (dd, J=7.2, 12.0 Hz, 1H), 4.15-4.09 (m, 1H), 4.08-4.03 (m, 1H), 3.31 (s, 2H), 3.19 (s, 4H), 2.92-2.81 (m, 1H), 2.62 (s, 1H), 2.08-1.97 (m, 2H), 1.86-1.78 (m, 2H), 1.17-1.05 (m, 1H), 0.62-0.51 (m, 2H), 0.50-0.38 (m, 2H). LCMS: (M+1: 588.2).

### Preparation of 4-[(9S)-9-cyclopropyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethyl-6-fluoronaphthalen-2-ol (Ex. 33)

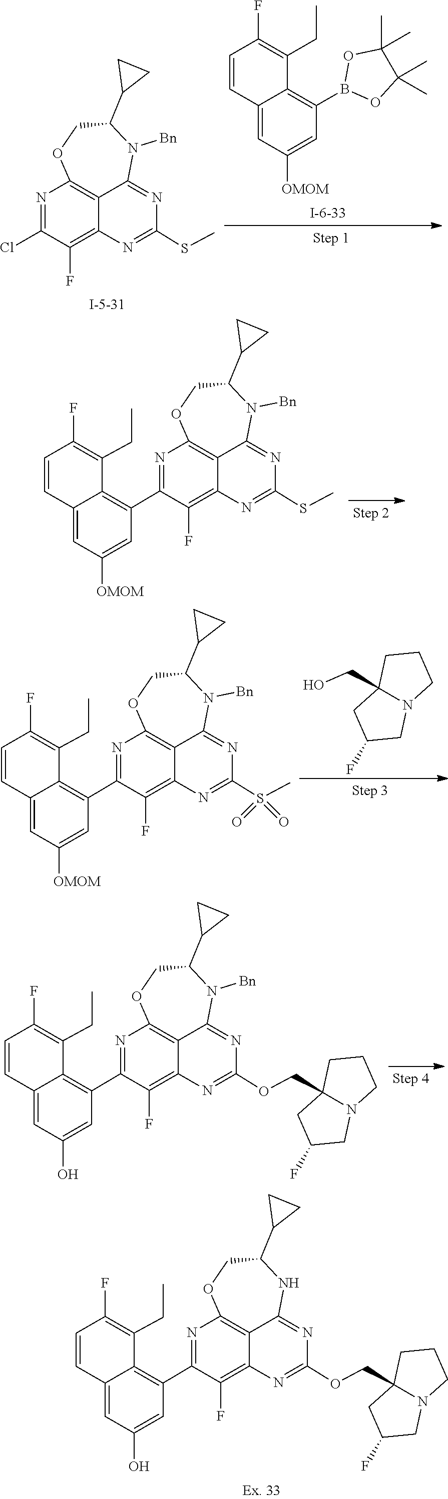

Step 1. A mixture of (12S)-13-benzyl-7-chloro-12-cyclopropyl-6-fluoro-3-methylsulfanyl-10-oxa-2,4,8,13-tetrazatricyclo[7.4.1.05,14]tetradeca-1,3,5,7,9 (14)-pentaene (630 mg, 1.51 mmol, 1 eq, intermediate I-5-31), 2-[8-ethyl-7-fluoro-3-(methoxymethoxy)-1-naphthyl]-4,4,5,5-tetramethyl-1,3,2-dioxaborolane (816 mg, 2.27 mmol, 1.5 eq), [2-(2-aminophenyl)phenyl]palladium(1+); bis(1-adamantyl)-butyl-phosphane; methanesulfonate (110 mg, 0.151 mmol, 0.1 eq), K₃PO₄ (962 mg, 4.53 mmol, 3 eq) in H₂O (2 mL) and dioxane (10 mL) was degassed and purged with N₂ 3 times, and then the mixture was stirred at 80° C. for 2 h under N₂ atmosphere. On completion, the mixture was concentrated in vacuum to give a residue. The residue was purified by column chromatography (SiO₂, Petroleum ether/Ethyl acetate=3:1 to 1:1) to give (12S)-13-benzyl-12-cyclopropyl-7-[8-ethyl-7-fluoro-3-(methoxymethoxy)-1-naphthyl]-6-fluoro-3-methylsulfanyl-10-oxa-2, 4, 8, 13-tetrazatricyclo [7.4.1.05, 14]tetradeca-1, 3, 5, 7, 9(14)-pentaene (890 mg, 1.45 mmol, 96% yield) as a yellow solid. LCMS: (M+1: 615.1).

Step 2. To a solution of (12S)-13-benzyl-12-cyclopropyl-7-[8-ethyl-7-fluoro-3-(methoxymethoxy)-1-naphthyl]-6-fluoro-3-methylsulfanyl-10-oxa-2, 4, 8, 13-tetrazatricyclo[7.4.1.05,14]tetradeca-1,3,5,7,9 (14)-pentaene (850 mg, 1.38 mmol, 1 eq) in DCM (10 mL) was added m-CPBA (701 mg, 3.46 mmol, 85% purity, 2.5 eq) at 0° C. The mixture was stirred at 25° C. for 1 h. On completion, the reaction mixture was quenched by addition Na₂SO₃ aqueous (10 mL) at 0° C., and then diluted with H₂O (20 mL) and extracted with DCM (10 mL*3). The combined organic layers were dried over Na₂SO₄, filtered and concentrated under reduced pressure to give (12S)-13-benzyl-12-cyclopropyl-7-[8-ethyl-7-fluoro-3-(methoxymethylsulfonyl-10-oxa-2,4,8,13-tetrazatricyclo[7.4.1.05,14]tetradeca-1,3,5,7,9 (14)-pentaene (930 mg, crude) as a yellow solid. LCMS: (M+1: 647.3).

Step 3. To a solution of [(2R,8S)-2-fluoro-1,2,3,5,6,7-hexahydropyrrolizin-8-yl]methanol (531 mg, 3.34 mmol, 3 eq) in toluene (10 mL) was added t-BuOK (374 mg, 3.34 mmol, 3 eq), the mixture was degassed and purged with N₂ 3 times. The mixture was added (12S)-13-benzyl-12-cyclopropyl-7-[8-ethyl-7-fluoro-3-(methoxymethoxy)-1-naphthyl]-6-fluoro-3-methylsulfonyl-10-oxa-2,4,8,13-tetrazatricyclo[7.4.1.05, 14]tetradeca-1, 3, 5, 7, 9(14)-pentaene (900 mg, 1.11 mmol, 1 eq) and stirred at 25° C. for 1 h. On completion, the reaction mixture was diluted with water (30 ml) and was extracted with EtOAc (20 mL*3). The combined organic layer was washed with brine (20 mL), dried over Na₂SO₄, filtered, and concentrated to give a residue. The residue was purified by column chromatography (SiO₂, DCM/MeOH=15:1 to 10:1) to give (12S)-13-benzyl-12-cyclopropyl-7-[8-ethyl-7-fluoro-3-(methoxymethoxy)-1-naphthyl]-6-fluoro-3-[[(2R,8S)-2-fluoro-1,2,3,5,6,7-hexahydropyrrolizin-8-yl]methoxy]-s10-oxa-2,4,8,13-tetrazatricyclo[7.4.1.05, 14]tetradeca-1, 3, 5, 7, 9(14)-pentaene (780 mg, 1.07 mmol, 97% yield) as a yellow solid. LCMS: (M+1: 726.2).

Step 4. A solution of (12S)-13-benzyl-12-cyclopropyl-7-[8-ethyl-7-fluoro-3-(methoxymethoxy)-1-naphthyl]-6-fluoro-3-[[(2R, 8S)-2-fluoro-1, 2, 3, 5, 6, 7-hexahydropyrrolizin-8-yl]methoxy]-10-oxa-2,4,8,13-tetrazatricyclo[7.4.1.05,14]tetradeca-1,3,5,7,9 (14)-pentaene (700 mg, 0.482 mmol, 1 eq) in i-PrOH (6 mL) was added Pd/C (70.0 mg, 0.066 mmol, 10% purity), Pd(OH)₂ (70.0 mg, 0.050 mmol, 10% purity) and HCl (1 M, 0.25 mL), degassed and purged with H₂ (15 psi) 3 times, and then the mixture was stirred at 25° C. for 3 h under H₂ atmosphere. On completion, the mixture was filtered with diatomaceous earth, the filtrate was concentrated in vacuum to give a residue. The residue was purified by column chromatography (SiO₂, DCM/MeOH=10:1 to 8:1) to give a crude product. Then the crude product was purified by Prep-HPLC purification (column: Phenomenex luna C18 150*25 mm*10 um; mobile phase: [water(FA)-ACN]; gradient: 20%-50% B over 10 min) to give 4-[(12S)-12-cyclopropyl-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.05,14]tetradeca-1,3,5,7,9 (14)-pentaen-7-yl]-5-ethyl-6-fluoro-naphthalen-2-ol (144.59 mg, 0.227 mmol, 67.08% yield, 100% purity, FA) as a white solid (Ex. 33). ¹H NMR (400 MHz, MeOD-d₄) δ=7.66 (dd, J=6.0, 9.2 Hz, 1H), 7.33-7.17 (m, 2H), 7.14-6.98 (m, 1H), 5.53-5.36 (m, 1H), 4.69-4.57 (m, 2H), 4.53-4.43 (m, 2H), 3.76-3.63 (m, 1H), 3.62-3.52 (m, 2H), 3.21-3.12 (m, 1H), 2.59-2.ss38 (m, 3H), 2.36-2.16 (m, 4H), 2.09-1.98 (m, 1H), 1.23-1.12 (m, 1H), 0.90-0.83 (m, 3H), 0.78-0.62 (m, 2H), 0.59-0.42 (m, 2H). LCMS: (M+1: 592.2).

### Preparation of 7-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-1,3-benzothiazol-2-amine (Ex. 40)

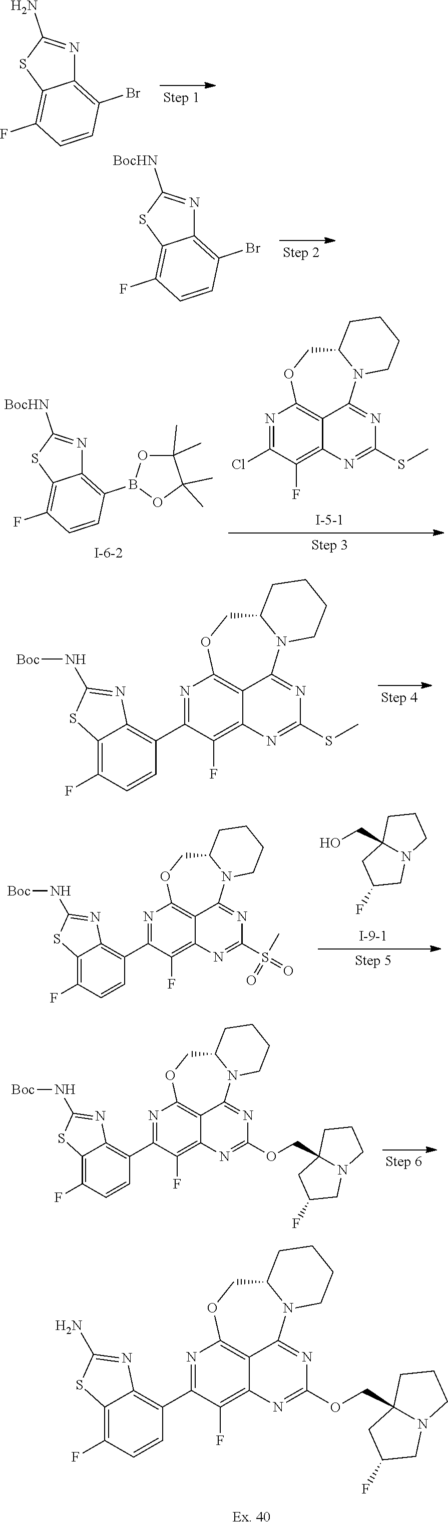

Step 1. To a solution of 4-bromo-7-fluoro-1,3-benzothiazol-2-amine (850 mg, 3.44 mmol, 1 eq) in dioxane (10 mL) was added Boc₂O (1.13 g, 5.16 mmol, 1.19 mL, 1.5 eq), DMAP (42.0 mg, 0.344 mmol, 0.1 eq) and DIPEA (1.33 g, 10.3 mmol, 1.80 mL, 3 eq) at 0° C. The mixture was stirred at 25 h for 16 h. On completion, the reaction mixture was diluted with H₂O (50 mL) and extracted with Ethyl Acetate (30 mL*3). The combined organic layers were dried over Na₂SO₄, filtered and concentrated under reduced pressure to give tert-butyl N-(4-bromo-7-fluoro-1,3-benzothiazol-2-yl)carbamate (1.36 g, 3.33 mmol, 97% yield, 85% purity) as a brown solid. ¹H NMR (400 MHz, DMSO-d6) δ=12.42 (s, 1H), 7.71-7.62 (m, 1H), 7.18-7.09 (m, 1H), 1.53-1.48 (s, 9H). LCMS: (M-56+1=292.7).

Step 2. A mixture of tert-butyl N-(4-bromo-7-fluoro-1,3-benzothiazol-2-yl)carbamate (500 mg, 1.44 mmol, 1 eq), Pd(PPh₃)₄ (166 mg, 0.144 mol, 0.1 eq), KOAc (424 mg, 4.32 mmol, 3 eq) and 4,4,5,5-tetramethyl-2-(4,4,5,5-tetramethyl-1,3,2-dioxaborolan-2-yl)-1,3,2-dioxaborolane (549 mg, 2.16 mmol, 1.5 eq) in dioxane (5 mL) was degassed and purged with N₂ 3 times, and then the mixture was stirred at 80° C. for 2 h under N₂ atmosphere. On completion, the reaction mixture was diluted with H₂O (60 mL) and extracted with EtOAc (30 mL*3). The combined organic layers were dried over Na₂SO₄, filtered, and concentrated under reduced pressure to give a residue. The residue was purified by column chromatography (SiO₂, Petroleum ether/Ethyl acetate=0/1 to 1/0) to give tert-butyl N-[7-fluoro-4-(4,4,5,5-tetramethyl-1,3,2-dioxaborolan-2-yl)-1,3-benzothiazol-2-yl]carbamate (550 mg, 1.39 mmol, 97% yield) as white solid.

Step 3. A mixture of (7S)-12-chloro-13-fluoro-16-methylsulfanyl-9-oxa-2,11,15,17-tetrazatetracyclo[8.7.1.02,7.014,18]octadeca-1 (17),10 (18),11,13,15-pentaene (70 mg, 0.205 mmol, 1 eq, intermediate I-5-1), tert-butyl N-[7-fluoro-4-(4,4,5,5-tetramethyl-1,3,2-dioxaborolan-2-yl)-1,3-benzothiazol-2-yl]carbamate (121 mg, 0.308 mmol, 1.5 eq.), Cs₂CO₃ (200 mg, 0.616 mmol, 3 eq), XPhos Pd G₄ (17.6 mg, 0.021 mmol, 0.1 eq) in dioxane (1 mL) and H₂O (0.2 mL) was degassed and purged with N₂ 3 times, and then the mixture was stirred at 80° C. for 2 h under N₂ atmosphere. On completion, the mixture was quenched with water (20 mL) and extracted with ethyl acetate (25 mL×3), the combined organic phase was dried over anhydrous sodium sulfate, filtered, and concentrated to give a residue. The residue was purified by column chromatography (SiO₂, Petroleum ether/Ethyl acetate=1:0 to 0:1) to give tert-butyl N-[7-fluoro-4-[(7S)-13-fluoro-16-methylsulfanyl-9-oxa-2,11,15,17-tetrazatetracyclo[8.7.1.02,7.014,18]octadeca-1 (17),10 (18),11,13,15-pentaen-12-yl]-1,3-benzothiazol-2-yl]carbamate (140 mg, 0.205 mmol, 100% yield, 84% purity) as white solid. LCMS: (M+1=573.2).

Step 4. To a solution of tert-butyl N-[7-fluoro-4-[(7S)-13-fluoro-16-methylsulfanyl-9-oxa-2,11,15,17-tetrazatetracyclo[8.7.1.02,7.014,18]octadeca-1 (17),10 (18),11,13,15-pentaen-12-yl]-1,3-benzothiazol-2-yl]carbamate (126 mg, 0.22 mmol, 1 eq) in DCM (0.5 mL) was added m-CPBA (118 mg, 0.55 mmol, 80% purity, 2.5 eq) at 0° C., and the mixture was stirred at 25° C. for 1 h. On completion, the reaction mixture was quenched by addition sat. Na₂SO₃ (2 mL) at 0° C., and then diluted with H₂O (20 mL) and extracted with DCM (10 mL*3). The combined organic layers were dried over Na₂SO₄, filtered, and concentrated under reduced pressure to give a residue. The residue was purified by column chromatography (SiO₂, Petroleum ether/THF=1/0 to 0/1) to give tert-butyl N-[7-fluoro-4-[(7S)-13-fluoro-16-methylsulfonyl-9-oxa-2,11,15,17-tetrazatetracyclo octadeca-1 (17),10 (18),11,13,15-pentaen-12-yl]-1,3-benzothiazol-2-yl]carbamate (110 mg, 0.181 mmol, 83% yield) as yellow solid. LCMS: (M+1=605.1).

Step 5. A mixture of [(2R,8S)-2-fluoro-1,2,3,5,6,7-hexahydropyrrolizin-8-yl]methanol (78.2 mg, 0.491 mmol, 3 eq), t-BuOK (55.1 mg, 0.491 mmol, 3 eq) in Toluene (1 mL) was degassed and purged with N₂ 3 times at 0° C., and then tert-butyl N-[7-fluoro-4-[(7S)-13-fluoro-16-methylsulfonyl-9-oxa-2,11,15,17-tetrazatetracyclo[8.7.1.02,7.014,18]octadeca-1 (17),10 (18),11,13,15-pentaen-12-yl]-1,3-benzothiazol-2-yl]carbamate (99 mg, 0.163 mmol, 1 eq) was added at 0° C., and then the mixture was stirred at 25° C. for 2 h under N₂ atmosphere. On completion, the mixture was quenched with water (20 mL) and extracted with ethyl acetate (25 mL×3), the combined organic phase was dried over anhydrous sodium sulfate, filtered, and concentrated to give a residue. The residue was purified by column chromatography (SiO₂, Petroleum ether/tetrahydrofuran=1:0 to 0:1) to give tert-butyl N-[7-fluoro-4-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaen-12-yl]-1,3-benzothiazol-2-yl]carbamate (80 mg, 0.117 mmol, 72% yield) as white solid. LCMS: (M+1=684.2).

Step 6. A solution of tert-butyl N-[7-fluoro-4-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo [8.7.1.02,7.014,18]octadeca-1 (17),10 (18),11,13,15-pentaen-12-yl]-1,3-benzothiazol-2-yl]carbamate (35 mg, 0.051 mmol, 1 eq) in DCM (0.5 mL) was added HCl/dioxane (4 M, 0.1 mL, 7.81 eq) and the mixture was stirred at 25° C. for 16 h. On completion, the mixture was concentrated in vacuum to give the residue. The residue was purified by prep-HPLC (column: Phenomenex luna C18 150*25 mm*10 um; mobile phase: [water(FA)-ACN]; B %: 14%-44%, 58 min) to give 7-fluoro-4-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaen-12-yl]-1,3-benzothiazol-2-amine (2.24 mg, 0.0038 mmol, 7.50% yield, 100% purity) as off-white solid (Ex. 40). ¹H NMR (400 MHz, DMSO-d₆) δ=7.94-7.86 (m, 2H), 7.34 (dd, J=5.6, 8.4 Hz, 1H), 7.03 (t, J=8.8 Hz, 1H), 5.37-5.19 (m, 1H), 5.16-5.07 (m, 1H), 4.52-4.39 (m, 2H), 4.16-3.95 (m, 2H), 3.93-3.85 (m, 1H), 3.11-3.06 (m, 2H), 3.05-2.99 (m, 2H), 2.85-2.79 (m, 1H), 2.15-2.10 (m, 1H), 2.05 (br s, 2H), 1.91-1.66 (m, 8H), 1.53-1.39 (m, 1H). LCMS: (M+1=584.2).

### Preparation of (2-(fluoromethylene)tetrahydro-1H-pyrrolizin-7a(5H)-yl)methanol (I-9-2)

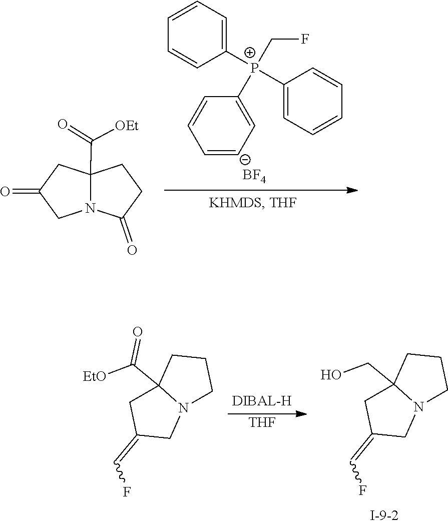

Step 1. A mixture of fluoromethyl(triphenyl)phosphonium; tetrafluoroborate (34.2 g, 89.5 mmol, 1.05 eq) in THF (180 mL) was degassed and purged with N₂ 3 times, and then KHMDS (1 M, 102 mL, 1.2 eq) was added at −68° C., and the mixture was stirred at −68° C. for 0.5 h under N₂ atmosphere. Then ethyl 3,6-dioxo-1,2,5,7-tetrahydropyrrolizine-8-carboxylate (18.0 g, 85.2 mmol, 1 eq) was added, the mixture was stirred at −68° C. for 3 h, then the mixture was stirred at 25° C. for 1 h. On completion, the mixture was quenched with sat. NH₄Cl (100 mL) and 3M HCl (200 mL), and then diluted with H₂O (50 mL) and extracted with EtOAc (80 mL*3). The combined organic layers were dried over Na₂SO₄, filtered and the filtrate was concentrated under reduced pressure to give a residue. The residue was purified by column chromatography (SiO₂, Petroleum ether/THF=1/0 to 0/1) to give Ethyl (6Z)-6-(fluoromethylene)-3-oxo-1,2,5,7-tetrahydropyrrolizine-8-carboxylate (9.60 g, 42.3 mmol, 49.57% yield) was obtained as yellow oil. ¹H NMR (400 MHz, CDCl₃) δ=6.70-6.42 (m, 1H), 4.35-4.27 (m, 1H), 4.24-4.14 (m, 2H), 3.73-3.64 (m, 1H), 3.34-3.23 (m, 1H), 2.87-2.70 (m, 1H), 2.68-2.58 (m, 1H), 2.49-2.37 (m, 2H), 2.21-2.05 (m, 1H), 1.30-1.20 (m, 3H).

Step 2. A mixture of ethyl (6Z)-6-(fluoromethylene)-3-oxo-1,2,5,7-tetrahydropyrrolizine-8-carboxylate (8.60 g, 37.9 mmol, 1 eq) in THF (90 mL) was degassed and purged with N₂ 3 times, then diisobutylaluminium hydride (2 M, 189 ml, 10 eq) was added at 0° C., the mixture was stirred at 25 for 1 h under N₂ atmosphere. On completion, the reaction mixture was quenched by addition sat. Na₂SO₃ (170 mL) at 0° C. Then diluted with H₂O (120 mL) and extracted with EtOAc (60 mL*3). The combined organic layers were dried over Na₂SO₄, filtered, and concentrated under reduced pressure to give a residue. The residue was purified by column chromatography (SiO₂, DCM:MeOH=1/0 to 0/1). [(6Z)-6-(fluoromethylene)-2,3,5,7-tetrahydro-1H-pyrrolizin-8-yl]methanol (2.38 g, 13.9 mmol, 36.73% yield) was obtained as yellow oil (I-9-2). ¹H NMR (400 MHz, CDCl₃) δ=6.68-6.35 (m, 1H), 3.77-3.67 (m, 1H), 3.62-3.50 (m, 1H), 3.35-3.19 (m, 2H), 3.08-3.01 (m, 1H), 2.68-2.56 (m, 1H), 2.55-2.47 (m, 1H), 2.45-2.34 (m, 1H), 2.01-1.92 (m, 1H), 1.89-1.82 (m, 1H), 1.81-1.64 (m, 3H).

Ex. 41 was prepared following General method D using above intermediate I-9-2 and (8aS)-5-[8-ethyl-7-fluoro-3-(methoxymethoxy)naphthalen-1-yl]-4-fluoro-2-(methanesulfonyl)-8,8a,9,10,11,12-hexahydro-7-oxa-1,3,6,12a-tetraazabenzo[4,5]cyclohepta[1,2,3-de]naphthalene (from the synthesis of Ex. 2) in Step 5.

### Preparation of N,N-bis(4-methoxybenzyl)-4-methyl-6-(tributylstannyl)-5-(trifluoromethyl)pyridin-2-amine (I-6-43)

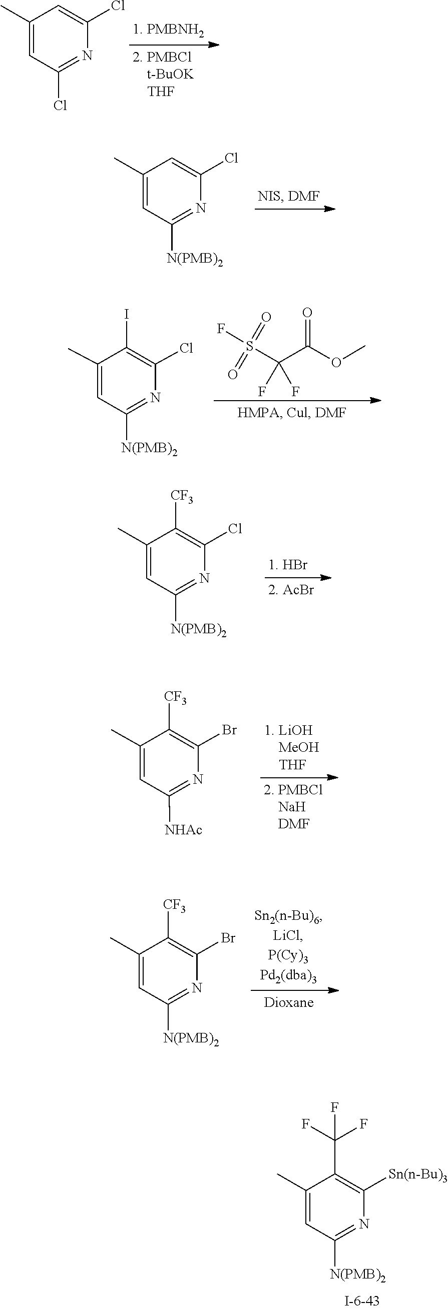

Step 1. To a mixture of 2,6-dichloro-4-methyl-pyridine (23.0 g, 141 mmol, 1 eq) in PMBNH₂ (121 g, 888 mmol, 115 mL, 6.26 eq) was stirred at 120° C. for 12 hours. On completion, the reaction mixture was diluted with Acetonitrile (100 mL). White solid was formed and filtered afford to 6-chloro-N-[(4-methoxyphenyl)methyl]-4-methyl-pyridin-2-amine (23.0 g, 87.5 mmol, 61% yield) as white solid. ¹H NMR (400 MHz, DMSO-d6) δ 7.49-7.41 (m, 3H), 6.99-6.92 (m, 3H), 6.38-6.26 (m, 111), 3.92 (s, 311), 3.76 (s, 3H), 2.12 (s, 2H); LCMS: (M+1=263.0).

To a solution of the above 6-chloro-N-[(4-methoxyphenyl)methyl]-4-methyl-pyridin-2-amine (23.0 g, 87.5 mmol, 1 eq) in THE (200 mL) was added PMB-Cl (16.4 g, 105 mmol, 14.2 mL, 1.2 eq) and t-BuOK (14.7 g, 131 mmol, 1.5 eq). The reaction mixture was stirred at 25° C. for 12 hours. On completion, the residue was diluted with water (120 mL) and extracted with EtOAc (2×120 mL). The combined organic layers were dried over Na₂SO₄, filtered, and concentrated in vacuo to give a residue. The residue was purified by column chromatography afford to 6-chloro-N,N-bis[(4-methoxyphenyl)methyl]-4-methyl-pyridin-2-amine (28.0 g, 73.1 mmol, 83% yield) as yellow oil. ¹H NMR (400 MHz, DMSO-d₆) δ 7.27-7.21 (m, 4H), 7.15 (d, J=8.8 Hz, 1H), 6.94-6.85 (m, 5H), 4.32 (s, 4H), 3.74 (s, 6H), 3.24 (s, 3H).

Step 2. To a solution of the above -chloro-N,N-bis[(4-methoxyphenyl)methyl]-4-methyl-pyridin-2-amine (27.0 g, 70.5 mmol, 1 eq) in DMF (300 mL) was added NIS (19.0 g, 84.6 mmol, 1.2 eq). The reaction mixture was stirred at 25° C. for 1 hour. On completion, the reaction mixture was diluted with water (100 mL). White solid was formed and filtered afford to 6-chloro-5-iodo-N,N-bis[(4-methoxyphenyl)methyl]-4-methyl-pyridin-2-amine (18.0 g, 35.3 mmol, 50% yield) as yellow solid. ¹H NMR (400 MHz, DMSO-d₆) δ 7.16 (d, J=8.8 Hz, 4H), 6.88 (d, J=8.8 Hz, 4H), 6.63 (s, 1H), 4.61 (s, 4H), 3.73 (s, 6H), 2.27 (s, 3H); LCMS: (M+1=509.1).

Step 3. To a mixture of 6-chloro-5-iodo-N,N-bis[(4-methoxyphenyl)methyl]-4-methyl-pyridin-2-amine (11.0 g, 21.6 mmol, 1 eq) in DMF (100 mL) was added CuI (10.2 g, 54.0 mmol, 2.5 eq) and HMPA (19.3 g, 108 mmol, 18.9 mL, 5 eq). The reaction mixture was stirred at 25° C. for 0.8 hour. Then to the reaction mixture was added methyl 2,2-difluoro-2-fluorosulfonyl-acetate (12.4 g, 64.8 mmol, 8.25 mL, 3 eq) dropwise at 90° C. The reaction mixture was stirred at 90° C. for 11.2 hours. On completion, the residue was diluted with water (100 mL) and extracted with EtOAc (2×100 mL). The combined organic layers were dried over Na₂SO₄, filtered, and concentrated in vacuo to give a residue. The residue was purified by column chromatography afford to 6-chloro-N,N-bis[(4-methoxyphenyl)methyl]-4-methyl-5-(trifluoromethyl)pyridin-2-amine (9.00 g, 19.9 mmol, 92% yield) as a yellow solid. ¹H NMR (400 MHz, DMSO-d₆) δ 7.40 (s, 1H), 4.69 (s, 1H), 3.61 (s, 3H), 3.43 (s, 2H), 2.88 (s, 3H), 1.38 (s, 9H), 1.21 (d, J=6.4 Hz, 3H); LCMS: (M+1=451.2).

Step 4. The mixture of 6-chloro-N,N-bis[(4-methoxyphenyl)methyl]-4-methyl-5-(trifluoromethyl) pyridin-2-amine (4.00 g, 8.87 mmol, 1 eq) in HBr (59.6 g, 243 mmol, 40 mL, 33% purity, 27.4 eq) was stirred at 90° C. for 1 hour. On completion, the reaction mixture was diluted with water (10 mL) and acidified with NaOH (15%) till pH=7-8. The residue was diluted with water (40 mL) and extracted with EtOAc (2×40 mL). The combined organic layers was dried over Na₂SO₄, filtered and concentrated afford to 6-chloro-4-methyl-5-(trifluoromethyl)pyridin-2-amine (2 g, crude) as brown solid. LCMS: (M+1=211.1).

The mixture of 6-chloro-N,N-bis[(4-methoxyphenyl)methyl]-4-methyl-5-(trifluoromethyl) pyridin-2-amine (2.00 g, 4.44 mmol, 1 eq) in Acetonitrile (30 mL) was added acetyl bromide (8.18 g, 66.5 mmol, 5.38 mL, 15 eq). The reaction mixture was stirred at 90° C. for 1 hour. On completion, the reaction mixture was quenched with EtOH (30 mL) and basified with NaOH (15%) till pH=7-8. The residue was diluted with water (30 mL) and extracted with EtOAc (2×40 mL). The combined organic layers was dried over Na₂SO₄, filtered and concentrated in vacuo afford to N-[6-bromo-4-methyl-5-(trifluoromethyl)-2-pyridyl]acetamide (2.00 g, crude) as yellow solid. LCMS: (M+1=297.1).

Step 5. The mixture of N-[6-bromo-4-methyl-5-(trifluoromethyl)-2-pyridyl]acetamide (2.00 g, 6.73 mmol, 1 eq) in THF (10 mL) and MeOH (10 mL) and H₂O (5 mL) was added LiOH·H₂O (564 mg, 13.4 mmol, 2 eq). The reaction mixture was stirred at 25° C. for 1 hour. On completion, the residue was diluted with water (30 mL) and extracted with EtOAc (2×30 mL). The combined organic layers were dried over Na₂SO₄, filtered, and concentrated in vacuo to give a residue. The residue was purified by column chromatography afford to 6-bromo-4-methyl-5-(trifluoromethyl)pyridin-2-amine (1.40 g, 5.49 mmol, 81% yield) as yellow solid. ¹H NMR (400 MHz, DMSO-d6) δ=7.00 (s, 2H), 6.29 (s, 1H), 2.29 (br d, J=3.1 Hz, 3H).

To a mixture of 6-bromo-4-methyl-5-(trifluoromethyl)pyridin-2-amine (1.50 g, 5.88 mmol, 1 eq) in THF (15 mL) was added t-BuOK (1.65 g, 14.7 mmol, 2.5 eq) and PMB-C₁ (2.21 g, 14.1 mmol, 1.92 mL, 2.4 eq). The reaction mixture was stirred at 25° C. for 12 hours. On completion. The residue was diluted with water (40 mL) and extracted with EtOAc (2×40 mL). The combined organic layers were dried over Na₂SO₄, filtered, and concentrated in vacuo to give a residue. The residue was purified by column chromatography afford to 6-bromo-N,N-bis[(4-methoxyphenyl)methyl]-4-methyl-5-(trifluoromethyl)pyridin-2-amine (1.30 g, 2.62 mmol, 44% yield) as yellow solid. ¹H NMR (400 MHz, DMSO-d₆) δ 7.24 (dd, J=2.8, 8.8 Hz, 4H), 6.97-6.93 (m, 4H), 6.70 (s, 1H), 4.73 (s, 4H), 3.79 (s, 6H), 2.39-2.35 (m, 3H); LCMS: (M+1=496.9).

Step 6. A mixture of 6-bromo-N,N-bis[(4-methoxyphenyl)methyl]-4-methyl-5-(trifluoromethyl)pyridin-2-amine (500 mg, 1.01 mmol, 1 eq), Pd₂(dba)₃ (92.4 mg, 101 mol, 0.1 eq), tributyl(tributylstannyl)stannane (1.76 g, 3.03 mmol, 1.52 mL, 3 eq), LiCl (214 mg, 5.05 mmol, 0.103 mL, 5 eq) and P(Cy)₃ (56.6 mg, 0.202 mmol, 0.066 mL, 0.2 eq) in dioxane (5 mL) was degassed and purged with N₂ 3 times, and then the mixture was stirred at 110° C. for 16 h under N₂ atmosphere. On completion, the reaction mixture was concentrated under reduced pressure to remove solvent. The residue was purified by column chromatography (SiO₂, Petroleum ether/Ethyl acetate=1/0 to 0/1). N,N-bis[(4-methoxyphenyl)methyl]-4-methyl-6-tributylstannyl-5-(trifluoromethyl)pyridin-2-anine (427 mg, 0.605 mmol, 60% yield) was obtained as yellow oil (I-6-43). LCMS: (M+1=707.0).

### Preparation of 6-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-4-methyl-5-(trifluoromethyl)pyridin-2-amine (Ex. 43)

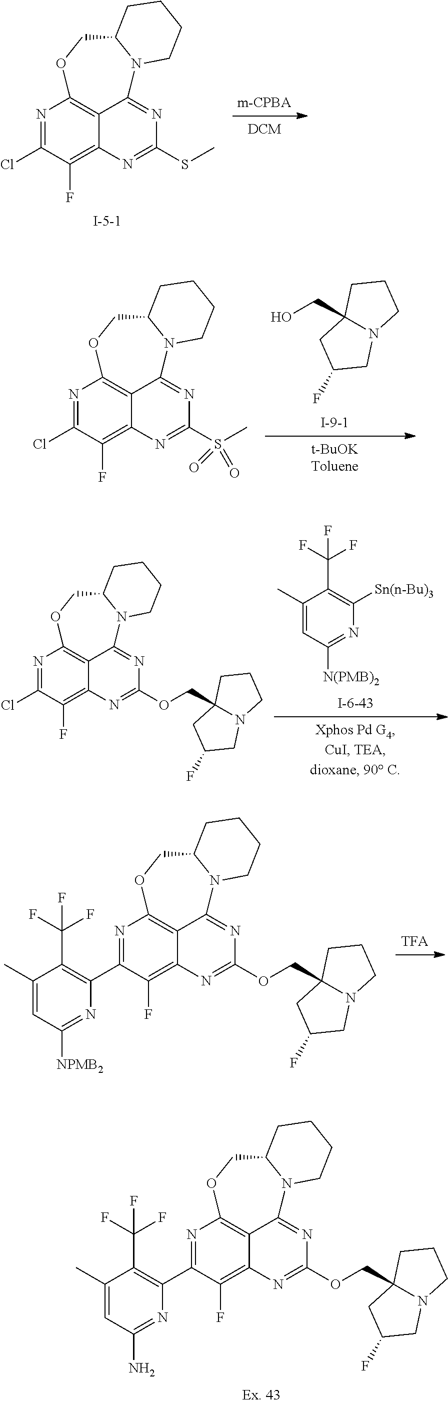

Step 1. To a solution of (7S)-12-chloro-13-fluoro-16-methylsulfanyl-9-oxa-2,11,15,17-tetrazatetracyclo[8.7.1.02,7.014,18]octadeca-1 (17),10 (18),11,13,15-pentaene (500 mg, 1.47 mmol, 1 eq, I-6-43) in DCM (10 mL) was added m-CPBA (745 mg, 3.67 mmol, 85% purity, 2.5 eq) at 0° C. The mixture was stirred at 25° C. for 1 h. On completion, the reaction mixture was quenched by addition Na₂SO₃ (5 mL) at 0° C., diluted with H₂O (20 mL) and extracted with DCM 30 mL (10 mL*3). The combined organic layers were dried over Na₂SO₄, filtered and the filtrate was concentrated under reduced pressure to give a residue. The residue was purified by column chromatography (SiO₂, Petroleum ether/THF=1/0 to 0/1) to give (7S)-12-chloro-13-fluoro-16-methylsulfonyl-9-oxa-2,11,15,17-tetrazatetracyclo[8.7.1.02,7.014,18]octadeca-1 (17),10 (18),11,13,15-pentaene (500 mg, 1.34 mmol, 91% yield) as white solid. LCMS: (M+1=372.9).

Step 2. A mixture of [(2R,8S)-2-fluoro-1,2,3,5,6,7-hexahydropyrrolizin-8-yl]methanol (288 mg, 1.81 mmol, 1.5 eq), t-BuOK (406 mg, 3.62 mmol, 3 eq) in Toluene (5 mL) was degassed and purged with N₂ 3 times at 0° C. and stirred for 0.5 h, and then (7S)-12-chloro-13-fluoro-16-methylsulfonyl-9-oxa-2,11,15,17-tetrazatetracyclo[8.7.1.02,7.014,18]octadeca-1 (17),10 (18),11,13,15-pentaene (450 mg, 1.21 mmol, 1 eq) was added at 0° C. and the mixture was stirred 25° C. for 0.5 h under N₂ atmosphere. On completion, the reaction mixture was diluted with H₂O 30 mL and extracted with EtOAc 60 mL (20 mL*3). The combined organic layers were dried over Na₂SO₄, filtered, and concentrated under reduced pressure to give a residue. The residue was purified by column chromatography (SiO₂, Petroleum ether/THF=1/0 to 0/1) to give (7S)-12-chloro-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaene (330 mg, 0.73 mmol, 61% yield) as white solid. LCMS: (M+1=452.0).

Step 3. A mixture of (7S)-12-chloro-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014, 18]octadeca-1 (17),10 (18),11,13,15-pentaene (100 mg, 0.221 mmol, 1 eq), N,N-bis[(4-methoxyphenyl)methyl]-4-methyl-6-tributylstannyl-5-(trifluoromethyl)pyridin-2-amine (156 mg, 0.221 mmol, 1 eq, preparation shown above), XPhos Pd G4 (19.0 mg, 0.022 mmol, 0.1 eq), CuI (42.1 mg, 0.221 mmol, 1 eq) and Triethylamine (44.8 mg, 0.443 mmol, 0.062 mL, 2 eq) in dioxane (2 mL) was degassed and purged with N₂ 3 times, and then the mixture was stirred at 90° C. for 16 h under N₂ atmosphere. On completion, the reaction mixture was concentrated under reduced pressure to remove solvent. The residue was purified by column chromatography (SiO₂, Petroleum ether/THF=I/O to 0/1) to give 6-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaen-12-yl]-N,N-bis[(4-methoxyphenyl)methyl]-4-methyl-5-(trifluoromethyl) pyridin-2-amine (50 mg, 0.060 mmol, 27% yield) as red oil. LCMS: (M+1=832.3).

Step 4. A solution of 6-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014, 18]octadeca-1 (17),10 (18),11,13,15-pentaen-12-yl]-N,N-bis[(4-methoxyphenyl)methyl]-4-methyl-5-(trifluoromethyl)pyridin-2-amine (45.0 mg, 0.054 mmol, 1 eq) in TFA (1.54 g, 13.5 mmol, 1.0 mL, 249 eq) was stirred at 60° C. for 2 h. On completion, the reaction mixture was concentrated under reduced pressure to remove solvent. The residue was purified by prep-HPLC (column: Phenomenex luna C18 150*25 mm*10 um; mobile phase: [water(FA)-ACN]; gradient: 16%-46% B over 10 min) to give 6-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaen-12-yl]-4-methyl-5-(trifluoromethyl)pyridin-2-amine (4.64 mg, 0.0068 mmol, 12.59% yield, 93.57% purity, FA) as yellow solid (Ex. 43). 11H NMR (400 MHz, DMSO-d6) δ=6.80-6.76 (m, 2H), 6.47 (s, 1H), 5.29 (s, 1H), 5.14-5.06 (m, 1H), 4.46-4.41 (m, 2H), 4.14-4.09 (m, 1H), 4.00-3.95 (m, 1H), 3.90 (br d, J=9.2 Hz, 1H), 3.12-3.06 (m, 3H), 3.01 (s, 2H), 2.86-2.77 (m, 2H), 2.68-2.66 (m, 1H), 2.36-2.34 (m, 3H), 2.14-2.10 (m, 1H), 2.04 (br d, J=2.4 Hz, 1H), 2.00 (br d, J=4.4 Hz, 1H), 1.86-1.82 (m, 2H), 1.79-1.74 (m, 3H), 1.53-1.42 (m, 1H); LCMS: (M+1=592.1).

### Preparation of (S)-3-chloro-5-(1-fluoro-11-(methylthio)-5,5a,6,7,8,9-hexahydro-4-oxa-3,9a,10,12-tetraazabenzo[4,5]cyclohepta[1,2,3-de]naphthalen-2-yl)-4-(trifluoromethyl)aniline (I-7-44)

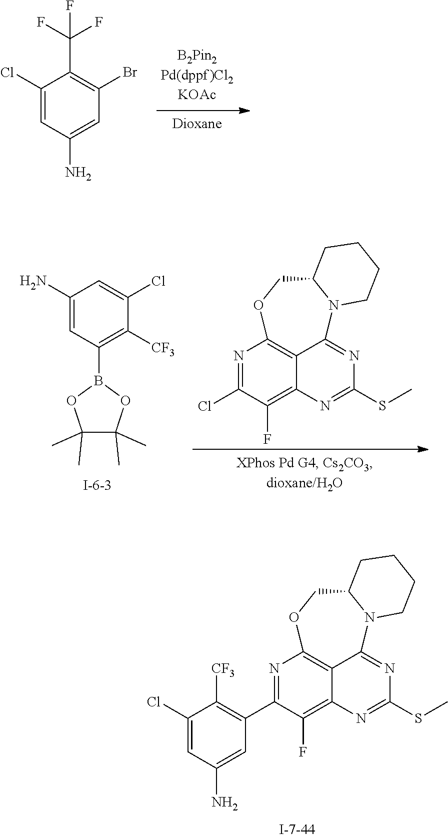 [SMILES: Nc1cc(Cl)c(C(F)(F)F)c(Br)c1 ; CC1(C)OB(c2cc(N)cc(Cl)c2C(F)(F)F)OC1(C)C ; CSc1nc2c3c(nc(Cl)c(F)c3n1)OC[C@@H]1CCCCN21 ; CSc1nc2c3c(nc(-c4cc(N)cc(Cl)c4C(F)(F)F)c(F)c3n1)OC[C@@H]1CCCCN21]

Step 1. A mixture of 3-bromo-5-chloro-4-(trifluoromethyl)aniline (1.5 g, 5.47 mmol, 1 eq), 4,4,5,5-tetramethyl-2-(4,4,5,5-tetramethyl-1,3,2-dioxaborolan-2-yl)-1,3,2-dioxaborolane (1.53 g, 6.01 mmol, 1.1 eq), Pd(dppf)Cl₂·CH₂Cl₂ (223 mg, 0.273 mmol, 0.05 eq), KOAc (1.61 g, 16.4 mmol, 3 eq) in dioxane (15 mL) was degassed and purged with N₂ 3 times, and then the mixture was stirred at 90° C. for 12 h under N₂ atmosphere. On completion, the mixture was concentrated to give a residue. The residue was purified by column chromatography on silica gel (PE:THF=100:1-100:5) to give 3-chloro-5-(4,4,5,5-tetramethyl-1,3,2-dioxaborolan-2-yl)-4-(trifluoromethyl)aniline (550 mg, 1.71 mmol, 45.8% yield) as yellow solid. ¹H NMR (400 MHz, CDCl₃-d) δ=6.74 (d, J=2.0 Hz, 1H), 6.57 (d, J=2.0 Hz, 1H), 4.31-3.59 (m, 2H), 1.36 (s, 12H); LCMS: (M+1: 321.7).

Step 2. A mixture of (7S)-12-chloro-13-fluoro-16-methylsulfanyl-9-oxa-2,11,15,17-tetrazatetracyclo[8.7.1.0²′7.014-18]octadeca-1 (17),10 (18),11,13,15-pentaene (125 mg, 0.367 mmol, 1 eq), 3-chloro-5-(4,4,5,5-tetramethyl-1,3,2-dioxaborolan-2-yl)-4-(trifluoromethyl)aniline (177 mg, 0.550 mmol, 1.5 eq), Cs₂CO₃ (359 mg, 1.10 mmol, 3 eq), XPhos Pd G4 (31.6 mg, 0.037 mmol, 0.1 eq) in dioxane (2 mL) and H₂O (0.4 mL) was degassed and purged with N₂ 3 times, and then the mixture was stirred at 80° C. for 2 h under N₂ atmosphere. On completion, the reaction mixture was diluted with H₂O (5 mL) and extracted with EtOAc (10 mL*3). The combined organic layers were dried over Na₂SO₄, filtered, and concentrated under reduced pressure to give a residue. The residue was purified by column chromatography (SiO₂, Petroleum ether/Ethyl acetate=1/0 to 0/1) to give 3-chloro-5-[(7S)-13-fluoro-16-methylsulfanyl-9-oxa-2,11,15,17-tetrazatetracyclo[8.7.1.0^{2,7}.0^{14,18}]octadeca-1 (17),10 (18),11,13,15-pentaen-12-yl]-4-(trifluoromethyl)aniline (96.0 mg, 0.192 mmol, 52% yield) as yellow oil; LCMS: (M+1=500.2).

Ex. 44 was synthesized following General Method D, using intermediates I-7-44 and I-5-1 in Step 4, followed by Step 5.

### Preparation of 7-bromo-5,8-difluoro-2-(methylthio)quinazolin-4 (3H)-one (I-1-2)

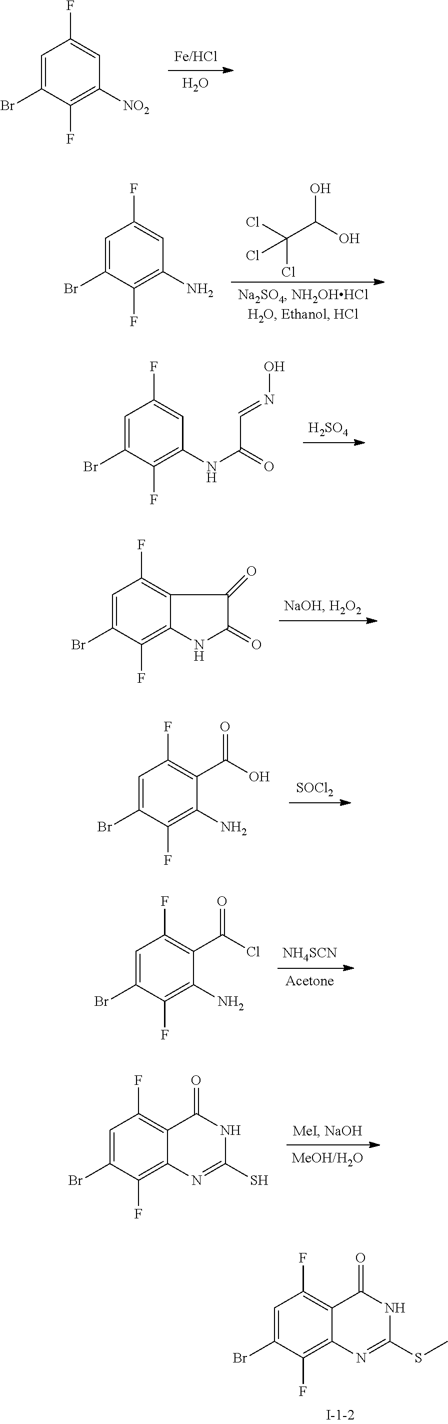

Ex. 45 was synthesized following General Method D, using above intermediate I-1-2 and intermediate I-2-35 in Step 1. Dichloromethane was used as the solvent in Step 2.

Ex. 50 was synthesized following General Method D, using I-2-16 in Step 1, and using XPhos Pd G4 and Cs₂CO₃ (instead of cataCXium® A Pd G3 and K₃PO₄) in Step 3.

Ex. 51 was synthesized following General Method D, using I-2-17 in Step 1, and using XPhos Pd G4 and Cs₂CO₃ (instead of cataCXium® A Pd G3 and K₃PO₄) in Step 3.

Ex. 52, Ex. 76, and Ex. 79 were synthesized following General Method D, using XPhos Pd G4 and Cs₂CO₃ (instead of cataCXium® A Pd G3 and K₃PO₄) in Step 3, and using intermediate I-9-2 in Step 5.

Ex. 53 and Ex. 113 were synthesized following General Method D, using I-2-18 in Step 1, and using XPhos Pd G4 and Cs₂CO₃ (instead of cataCXium® A Pd G3 and K₃PO₄) in Step 3.

Ex. 54 was synthesized following General Method D, using intermediate I-2-19 in Step 1, and using XPhos Pd G4 and Cs₂CO₃ (instead of cataCXium® A Pd G3 and K₃PO₄) in Step 3.

### Preparation of 5-ethynyl-6-fluoro-4-[(9S)-4-fluoro-2-{1[(2R,7aS)-2-fluorotetrahydro-1H-pyrrolizin-7a(5H)-yl]methoxy}-9-methyl-10-(propan-2-yl)-9,10-dihydro-8H-7-oxa-1,3,6,10-tetraazacyclohepta[1,2,3-de]naphthalen-5-yl]naphthalen-2-ol (Ex. 56)

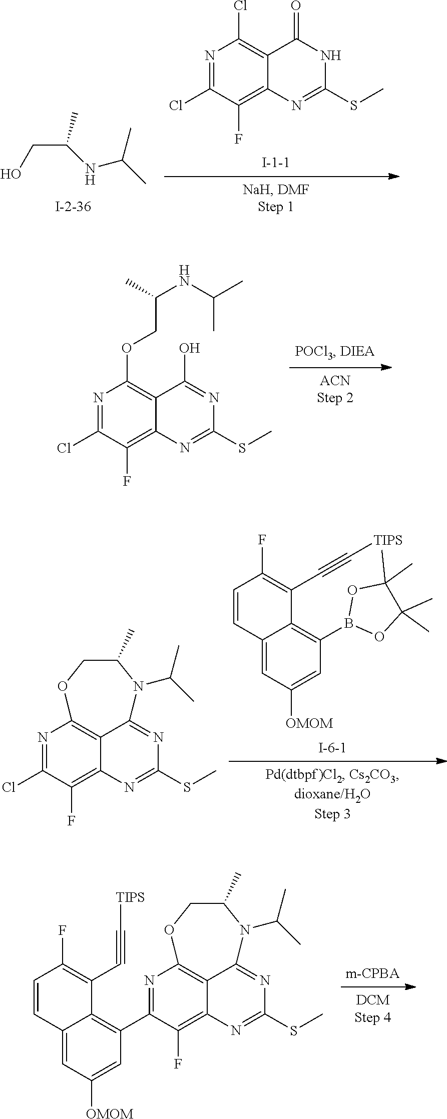

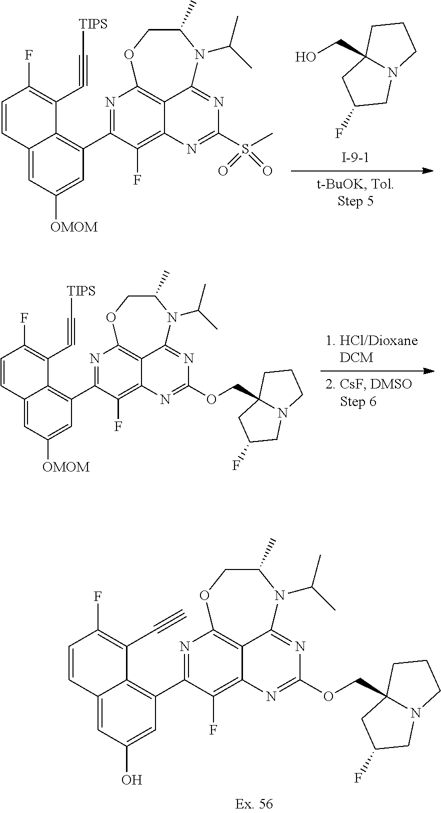

Step 1. To a solution of (2S)-2-(isopropylamino)propan-1-ol (600 mg, 5.12 mmol, 1 eq, I-2-36) in DMF (15 mL) was added NaH (409 mg, 10.2 mmol, 60% purity, 2 eq) at 0° C. and stirred at 0° C. for 0.5 h. Then the mixture was added 5,7-dichloro-8-fluoro-2-methylsulfanyl-3H-pyrido[4,3-d]pyrimidin-4-one (1.00 g, 3.58 mmol, 0.7 eq, I-1-1) at 0° C. The mixture was stirred at 25° C. for 2 hr. On completion, the mixture was quenched by water (50 mL) at 0° C. The mixture was filtered and the filter cake was dried over vacuum to give 7-chloro-8-fluoro-5-[(2S)-2-(isopropylamino)propoxy]-2-methylsulfanyl-pyrido[4,3-d]pyrimidin-4-ol (700 mg, 1.94 mmol, 38% yield) as yellow solid. ¹H NMR (400 MHz, MeOD-d₄) δ=4.75-4.61 (m, 1H), 4.43-4.30 (m, 1H), 3.86-3.72 (m, 1H), 3.63-3.46 (m, 1H), 2.57-2.49 (m, 3H), 1.46-1.32 (m, 9H); LCMS: (M+1:361.0).

Step 2. To a solution of 7-chloro-8-fluoro-5-[(2S)-2-(isopropylamino)propoxy]-2-methylsulfanyl-pyrido[4,3-d]pyrimidin-4-ol (650 mg, 1.80 mmol, 1 eq), DIEA (2.10 g, 16.2 mmol, 2.82 mL, 9 eq) in ACN (8 mL) was added POCl₃ (828 mg, 5.40 mmol, 0.503 mL, 3 eq). The mixture was stirred at 25° C. for 2 hr. On completion, the mixture was concentrated to give a residue. The residue was purified by column chromatography (SiO₂, Petroleum ether/Ethyl acetate=1:0 to 1:1) to give (12S)-7-chloro-6-fluoro-13-isopropyl-12-methyl-3-methylsulfanyl-10-oxa-2,4,8,13-tetrazatricyclo[7.4.1.05,14]tetradeca-1,3,5,7,9 (14)-pentaene (480 mg, 1.40 mmol, 78% yield) as yellow solid. LCMS: (M+1:343.1).

Step 3. A mixture of (12S)-7-chloro-6-fluoro-13-isopropyl-12-methyl-3-methylsulfanyl-10-oxa-2,4,8,13-tetrazatricyclo[7.4.1.05,14]tetradeca-1,3,5,7,9 (14)-pentaene (440 mg, 1.28 mmol, 1 eq), 2-[2-fluoro-6-(methoxymethoxy)-8-(4,4,5,5-tetramethyl-1,3,2-dioxaborolan-2-yl)-1-naphthyl]ethynyl-triisopropyl-silane (986 mg, 1.93 mmol, 1.5 eq, I-6-1), Cs₂CO₃ (1.25 g, 3.85 mmol, 3 eq), di-tert-butyl(cyclopentyl)phosphane; dichloropalladium; iron (83.6 mg, 0.128 mmol, 0.1 eq) in dioxane (5 mL) and H₂O (1 mL) was degassed and purged with N₂ 3 times, and then the mixture was stirred at 80° C. for 2 h under N₂ atmosphere. On completion, the mixture was filtered and concentrated to give a residue. The residue was purified by column chromatography (SiO₂, Petroleum ether/Ethyl acetate=1:0 to 3:1) to give 2-[2-fluoro-8-[(12S)-6-fluoro-13-isopropyl-12-methyl-3-methylsulfanyl-10-oxa-2,4,8,13-tetrazatricyclo[7.4.1.05,14]tetradeca-1,3,5,7,9 (14)-pentaen-7-yl]-6-(methoxymethoxy)-1-naphthyl]ethynyl-triisopropyl-silane (850 mg, 1.23 mmol, 96% yield) as yellow oil. LCMS: (M+1:693.2).

Step 4. To a solution of 2-[2-fluoro-8-[(12S)-6-fluoro-13-isopropyl-12-methyl-3-methylsulfanyl-10-oxa-2,4,8,13-tetrazatricyclo[7.4.1.05,14]tetradeca-1,3,5,7,9 (14)-pentaen-7-yl]-6-(methoxymethoxy)-1-naphthyl]ethynyl-triisopropyl-silane (800 mg, 1.15 mmol, 1 eq) in DCM (8 mL) was added m-CPBA (703 mg, 3.46 mmol, 85% purity, 3 eq) at 0° C. The mixture was stirred at 25° C. for 2 hr. On completion, the mixture was quenched with sat. Na₂SO₃ (50 mL) at 0° C. and extracted with ethyl acetate (25 mL×3), the combined organic phase was dried over anhydrous Na₂SO₄, filtered and the filtrate was concentrated to give 2-[2-fluoro-8-[(12S)-6-fluoro-13-isopropyl-12-methyl-3-methylsulfonyl-10-oxa-2,4,8,13-tetrazatricyclo[7.4.1.05,14]tetradeca-1,3,5,7,9 (14)-pentaen-7-yl]-6-(methoxymethoxy)-1-naphthyl]ethynyl-triisopropyl-silane (830 mg, crude) as yellow solid. LCMS: (M+1:725.3).

Step 5. To a solution of [(2R,8S)-2-fluoro-1,2,3,5,6,7-hexahydropyrrolizin-8-yl]methanol (546 mg, 3.43 mmol, 3 eq, I-9-1), t-BuOK (385 mg, 3.43 mmol, 3 eq) in Toluene (10 mL) was added 2-[2-fluoro-8-[(12S)-6-fluoro-13-isopropyl-12-methyl-3-methylsulfonyl-10-oxa-2,4,8,13-tetrazatricyclo[7.4.1.05,14]tetradeca-1,3,5,7,9 (14)-pentaen-7-yl]-6-(methoxymethoxy)-1-naphthyl]ethynyl-triisopropyl-silane (830 mg, 1.14 mmol, 1 eq). The mixture was stirred at 25° C. for 1 hr. On completion, the mixture was diluted with water (50 mL) and extracted with ethyl acetate (30 mL×3). The combined organic phase was washed with water (50 mL×2) and dried over Na₂SO₄, filtered, and the filtrate was concentrated to give a residue. The residue was purified by column chromatography (SiO₂, Petroleum ether/Ethyl acetate=1:0 to 0:1) to give 2-[2-fluoro-8-[(12S)-6-fluoro-3-[[(2R,8S)-2-fluoro-1,2,3,5,6,7-hexahydropyrrolizin-8-yl]methoxy]-13-isopropyl-12-methyl-10-oxa-2,4,8,13-tetrazatricyclo [7.4.1.05,14]tetradeca-1,3,5,7,9 (14)-pentaen-7-yl]-6-(methoxymethoxy)-1-naphthyl]ethynyl-triisopropyl-silane (350 mg, 0.435 mmol, 38% yield) as yellow solid. ¹H NMR (400 MHz, MeOD-d₄) δ=7.98-7.91 (m, 1H), 7.67-7.62 (m, 1H), 7.44-7.27 (m, 2H), 5.79-5.64 (m, 1H), 5.35-5.30 (m, 2H), 4.77-4.63 (m, 2H), 4.39-4.30 (m, 2H), 4.21-4.06 (m, 2H), 3.49-3.48 (m, 1H), 3.51-3.48 (m, 2H), 2.34-2.08 (m, 4H), 2.05-1.83 (m, 4H), 1.45-1.30 (m, 11H), 0.99-0.86 (m, 21H); LCMS: (M+1:804.4).

Step 6-1. To a solution of 2-[2-fluoro-8-[(12S)-6-fluoro-3-[[(2R,8S)-2-fluoro-1,2,3,5,6,7-hexahydropyrrolizin-8-yl]methoxy]-13-isopropyl-12-methyl-10-oxa-2,4,8,13-tetrazatricyclo[7.4.1.05,14]tetradeca-1,3,5,7,9 (14)-pentaen-7-yl]-6-(methoxymethoxy)-1-naphthyl]ethynyl-triisopropyl-silane (50.0 mg, 0.062 mmol, 1 eq) in DCM (0.5 ml) was added HCl/Dioxane (4 M, 0.1 mL, 6.43 eq). The mixture was stirred at 25° C. for 1 hr. On completion, the mixture was concentrated to give 6-fluoro-4-[(12S)-6-fluoro-3-[[(2R,8S)-2-fluoro-1,2,3,5,6,7-hexahydropyrrolizin-8-yl]methoxy]-13-isopropyl-12-methyl-10-oxa-2,4,8,13-tetrazatricyclo[7.4.1.05,14]tetradeca-1,3,5,7,9 (14)-pentaen-7-yl]-5-(2-triisopropylsilylethynyl)naphthalen-2-ol (47.0 mg, 0.062 mmol, 99% yield) as yellow oil. LCMS: (M+1:760.4).

Step 6-2. To a solution of the above 6-fluoro-4-[(12S)-6-fluoro-3-[[(2R,8S)-2-fluoro-1,2,3,5,6,7-hexahydropyrrolizin-8-yl]methoxy]-13-isopropyl-12-methyl-10-oxa-2,4,8,13-tetrazatricyclo[7.4.1.05,14]tetradeca-1,3,5,7,9 (14)-pentaen-7-yl]-5-(2-triisopropylsilylethynyl)naphthalen-2-ol (47.0 mg, 0.062 mmol, 1 eq) in DMSO (0.5 mL) was added CsF (28.1 mg, 0.185 mmol, 3 eq). The mixture was stirred at 25° C. for 1 hr. On completion, the mixture was filtered and concentrated to give a residue. The residue was purified by Prep-HPLC purification (Phenomenex luna C18 150*25 mm*10 um; mobile phase: [water(FA)-ACN]; gradient: 19%-49% B over 10 min) to give 5-ethynyl-6-fluoro-4-[(12S)-6-fluoro-3-[[(2R,8S)-2-fluoro-1,2,3,5,6,7-hexahydropyrrolizin-8-yl]methoxy]-13-isopropyl-12-methyl-10-oxa-2,4,8,13-tetrazatricyclo [7.4.1.05,14]tetradeca-1,3,5,7,9 (14)-pentaen-7-yl]naphthalen-2-ol (18.28 mg, 0.030 mmol, 48.97% yield, FA) as white solid (Ex. 56). ¹H NMR (400 MHz, MeOD-d₄) δ=7.89-7.80 (m, 1H), 7.36-7.28 (m, 2H), 7.26-7.14 (m, 1H), 5.79-5.62 (m, 1H), 5.49-5.27 (m, 1H), 4.70-4.66 (m, 1H), 4.60-4.54 (m, 1H), 4.44-4.27 (m, 4H), 3.53-3.42 (m, 3H), 3.19-3.12 (m, 1H), 2.51-2.30 (m, 2H), 2.28-2.18 (m, 1H), 2.17-2.06 (m, 2H), 2.02-1.91 (m, 1H), 1.45-1.40 (m, 3H), 1.33 (br s, 6H); LCMS: (M+1: 604.2).

### Preparation of 4-[(9S)-10-benzyl-4-fluoro-2-{[(2R,7aS)-2-fluorotetrahydro-1H-pyrrolizin-7a(5H)-yl]methoxy}-9-methyl-9,10-dihydro-8H-7-oxa-1,3,6,10-tetraazacyclohepta[1,2,3-de]naphthalen-5-yl]-5-ethynyl-6-fluoronaphthalen-2-ol (Ex. 74)

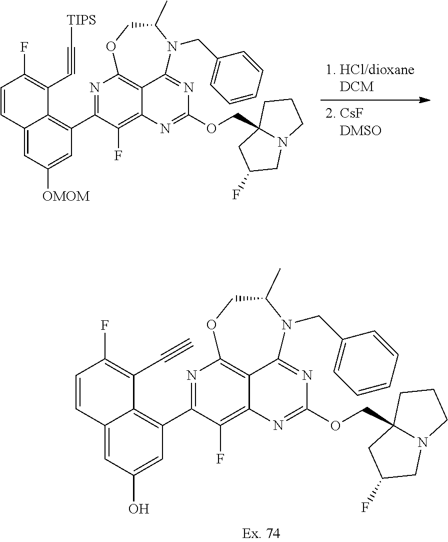

To a solution of 2-[8-[(12S)-13-benzyl-6-fluoro-3-[[(2R,8S)-2-fluoro-1,2,3,5,6,7-hexahydropyrrolizin-8-yl]methoxy]-12-methyl-10-oxa-2,4,8,13-tetrazatricyclo [7.4.1.05,14]tetradeca-1,3,5,7,9 (14)-pentaen-7-yl]-2-fluoro-6-(methoxymethoxy)-1-naphthyl]ethynyl-triisopropyl-silane (22 mg, 0.0258 mmol, 1 eq, from the synthesis of Ex. 46) in DCM (1 mL) was added HCl/dioxane (4 M, 0.55 mL, 85.2 eq). The mixture was stirred at 25° C. for 1 h. On completion, the reaction mixture was concentrated under reduced pressure to give 4-[(12S)-13-benzyl-6-fluoro-3-[[(2R,8S)-2-fluoro-1,2,3,5,6,7-hexahydropyrrolizin-8-yl]methoxy]-12-methyl-10-oxa-2,4,8,13-tetrazatricyclo[7.4.1.05,14]tetradeca-1,3,5,7,9 (14)-pentaen-7-yl]-6-fluoro-5-(2-triisopropylsilylethynyl)naphthalen-2-ol (20 mg, 0.025 mmol, 96% yield) as yellow oil. LCMS: (M+1=808.4).

To a solution of 4-[(12S)-13-benzyl-6-fluoro-3-[[(2R,8S)-2-fluoro-1,2,3,5,6,7-hexahydropyrrolizin-8-yl]methoxy]-12-methyl-10-oxa-2,4,8,13-tetrazatricyclo[7.4.1.05,14]tetradeca-1,3,5,7,9 (14)-pentaen-7-yl]-6-fluoro-5-(2-triisopropylsilylethynyl) naphthalen-2-ol (20 mg, 0.025 mmol, 1 eq) in DMSO (0.5 mL) was added CsF (45.1 mg, 0.297 mmol, 12 eq). The mixture was stirred at 25° C. for 1 h. On completion, the mixture was filtrated to remove CsF. The filtrate was purified by prep-HPLC (column: Phenomenex luna C18 150*25 mm*10 um; mobile phase: [water(FA)-ACN]; B %: 20%-50%, 10 min) to give 4-[(12S)-13-benzyl-6-fluoro-3-[[(2R,8S)-2-fluoro-1,2,3,5,6,7-hexahydropyrrolizin-8-yl]methoxy]-12-methyl-10-oxa-2,4,13-triazatricyclo[7.4.1.05, 14]tetradeca-1,3,5,7,9 (14)-pentaen-7-yl]-5-ethynyl-6-fluoro-naphthalen-2-ol (1.25 mg, 0.0017 mmol, 7.03% yield, 96.97% purity, FA) as off-white solid (Ex. 74). ¹H NMR (400 MHz, MeOD-d₄) δ=7.89-7.81 (m, 1H), 7.42-7.16 (m, 8H), 5.67-5.50 (m, 1H), 5.38-5.18 (m, 2H), 4.68-4.49 (m, 4H), 4.26-4.15 (m, 2H), 4.11-4.02 (m, 1H), 3.49-3.45 (m, 1H), 3.10-3.01 (m, 1H), 2.35-1.94 (m, 6H), 1.87-1.73 (m, 1H), 1.47-1.40 (m, 3H); LCMS: (M+1=652.3).

Ex. 75 was synthesized following General Method D, using intermediate I-2-12 in Step 1.

### Preparation of (2R,7aS)-7a-({[(9S)-5-(8-ethynyl-7-fluoro-3-hydroxynaphthalen-1-yl)-4-fluoro-9-methyl-9,10-dihydro-8H-7-oxa-1,3,6,10-tetraazacyclohepta[1,2,3-de]naphthalen-2-yl]oxy}methyl)-2-fluoro-4-methylhexahydro-1H-pyrrolizin-4-ium (Ex. 77)

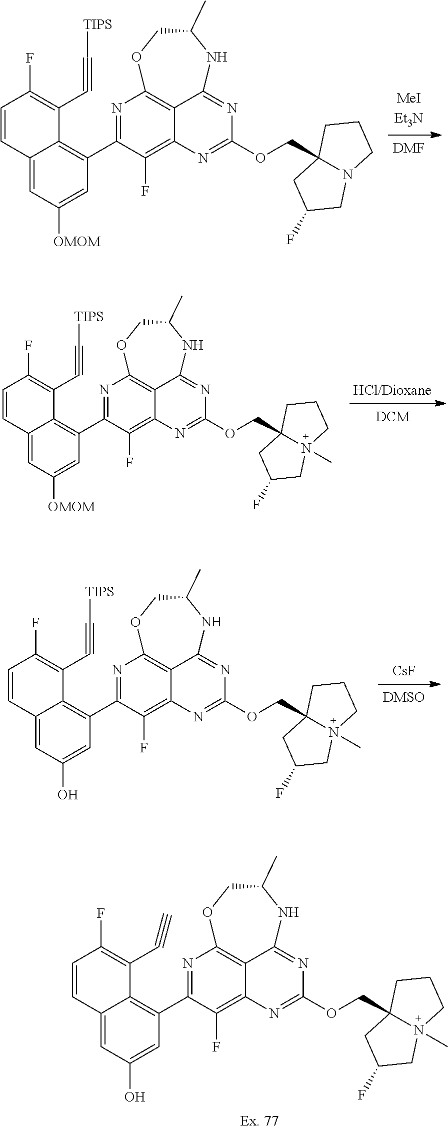

Step 1. To a solution of 2-[2-fluoro-8-[(12S)-6-fluoro-3-[[(2R,8S)-2-fluoro-1,2,3,5,6,7-hexahydropyrrolizin-8-yl]methoxy]-12-methyl-10-oxa-2,4,8,13-tetrazatricyclo [7.4.1.05,14]tetradeca-1,3,5,7,9 (14)-pentaen-7-yl]-6-(methoxymethoxy)-1-naphthyl]ethynyl-triisopropyl-silane (20 mg, 0.0263 mmol, 1 eq, from the synthesis of Ex. 46) in DMF (1 ml) was added Mel (11.1 mg, 0.079 mmol, 0.005 mL, 3 eq) and Triethylamine (15.9 mg, 0.157 mmol, 0.022 mL, 6 eq). The mixture was stirred at 25° C. for 1 h. On completion, the reaction mixture was diluted with H₂O (15 mL) and extracted with EtOAc (10 mL*3). The combined organic layers were dried over Na₂SO₄, filtered and concentrated under reduced pressure to give (2R,7aS)-2-fluoro-7a-[({(9S)-4-fluoro-5-[7-fluoro-3-(methoxymethoxy)-8-{[tri(propan-2-yl)silyl]ethynyl}naphthalen-1-yl]-9,10-dimethyl-9,10-dihydro-8H-7-oxa-1,3,6,10-tetraazacyclohepta[1,2,3-de]naphthalen-2-yl}oxy)methyl]-4-methylhexahydro-1H-pyrrolizin-4-ium (20 mg, 0.0258 mmol, 98% yield) as brown oil. LCMS: (M+1=776.5).

Step 2. To a solution of 2-[2-fluoro-8-[(12S)-6-fluoro-3-[[(2R,8S)-2-fluoro-1,2,3,5,6,7-hexahydropyrrolizin-8-yl]methoxy]-12,13-dimethyl-10-oxa-2,4,8,13-tetrazatricyclo [7.4.1.05,14]tetradeca-1,3,5,7,9 (14)-pentaen-7-yl]-6-(methoxymethoxy)-1-naphthyl]ethynyl-triisopropyl-silane (20 mg, 0.0258 mmol, 1 eq) in DCM (1 mL) was added HCl/dioxane (4 M, 0.2 mL, 31.0 eq). The mixture was stirred at 25° C. for 1 h. On completion, the reaction mixture was concentrated under reduced pressure to remove solvent to give (2R,7aS)-2-fluoro-7a-({[(9S)-4-fluoro-5-(7-fluoro-3-hydroxy-8-{[tri(propan-2-yl)silyl]ethynyl}naphthalen-1-yl)-9,10-dimethyl-9,10-dihydro-8H-7-oxa-1,3,6,10-tetraazacyclohepta[1,2,3-de]naphthalen-2-yl]oxy}methyl)-4-methylhexahydro-1H-pyrrolizin-4-ium (18 mg, 0.0246 mmol, 95% yield) as brown solid. LCMS: (M+1=732.2).

Step 3. To a solution of 6-fluoro-4-[(12S)-6-fluoro-3-[[(2R,8S)-2-fluoro-1,2,3,5,6,7-hexahydropyrrolizin-8-yl]methoxy]-12,13-dimethyl-10-oxa-2,4,8,13-tetrazatricyclo [7.4.1.05,14]tetradeca-1,3,5,7,9 (14)-pentaen-7-yl]-5-(2-triisopropylsilylethynyl) naphthalen-2-ol (18 mg, 0.0246 mmol, 1 eq) in DMSO (1 mL) was added CsF (44.8 mg, 0.295 mmol, 12 eq). The mixture was stirred at 25° C. for 1 h. On completion, the mixture was filtered to remove CsF. The filtrate was purified by prep-HPLC (column: Phenomenex luna C18 150*25 mm*10 um; mobile phase: [water(FA)-ACN]; gradient: 11%-41% B over 10 min) to give (2R,7aS)-7a-({[(9S)-5-(8-ethynyl-7-fluoro-3-hydroxynaphthalen-1-yl)-4-fluoro-9-methyl-9,10-dihydro-8H-7-oxa-1,3,6,10-tetraazacyclohepta[1,2,3-de]naphthalen-2-yl]oxy}methyl)-2-fluoro-4-methylhexahydro-1H-pyrrolizin-4-ium (0.91 mg, 0.0015 mmol, 5.95% yield, 100% purity, FA) as an off-white solid (Ex. 77). ¹H NMR (400 MHz, MeOD-d₄) δ=7.88-7.83 (m, 1H), 7.35-7.30 (m, 2H), 7.22-7.19 (m, 1H), 5.70-5.49 (m, 1H), 4.61-4.59 (m, 4H), 4.53-4.43 (m, 2H), 4.34-4.16 (m, 2H), 4.11-4.01 (m, 2H), 3.92-3.83 (m, 1H), 3.54-3.47 (m, 1H), 3.36-3.35 (m, 1H), 3.05-2.89 (m, 1H), 2.70-2.58 (m, 1H), 2.32 (br s, 4H), 1.46-1.41 (m, 3H); LCMS: (M+1=576.1).

### Preparation of 5-chloro-1-(tetrahydro-2H-pyran-2-yl)-4-(4,4,5,5-tetramethyl-1,3,2-dioxaborolan-2-yl)-1H-indazole (I-6-4)

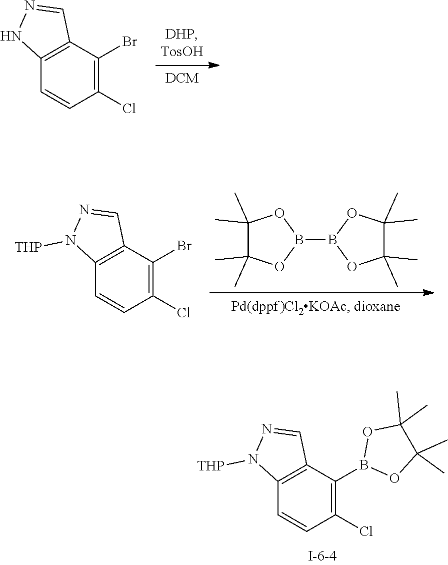

Step 1. To a solution of 4-bromo-5-chloro-1H-indazole (1 g, 4.32 mmol, 1 eq) in DCM (10 mL) was added PTSA (37.2 mg, 0.216 mmol, 0.05 eq) and 3,4-Dihydropyran (1.09 g, 12.9 mmol, 3 eq). The mixture was stirred at 25° C. for 1 h. On completion, the reaction mixture was diluted with H₂O (10 mL) and extracted with DCM (10 mL×3). The combined organic layers were washed with brine 10 mL (5 mL×2), dried over anhydrous sodium sulfate, filtered, and concentrated to give a residue. The residue was purified by prep-HPLC (column: Phenomenex luna C18 250*50 mm*15 um; mobile phase: [water (NH₄HCO₃)-ACN]; gradient: 70%-90% B over 25 min) to give 4-bromo-5-chloro-1-tetrahydropyran-2-yl-indazole (1.30 g, 3.95 mmol, 92% yield) as yellow oil. ¹H NMR (400 MHz, DMSO-d₆) δ=8.11 (s, 1H), 7.84-7.80 (m, 1H), 7.60 (d, J=8.8 Hz, 1H), 5.94-5.85 (m, 1H), 3.90-3.84 (m, 1H), 3.79-3.71 (m, 1H), 2.42-2.30 (m, 1H), 2.05-1.96 (m, 2H), 1.78-1.69 (m, 1H), 1.60-1.55 (m, 2H); LCMS: (M+23: 337.3).

Step 2. A mixture of 4,4,5,5-tetramethyl-2-(4,4,5,5-tetramethyl-1,3,2-dioxaborolan-2-yl)-1,3,2-dioxaborolane (1.57 g, 6.18 mmol, 1.5 eq), 4-bromo-5-chloro-1-tetrahydropyran-2-yl-indazole (1.30 g, 4.12 mmol, 1 eq), Pd(dppf)Cl₂ (301 mg, 0.411 mmol, 0.1 eq), KOAc (1.21 g, 12.3 mmol, 3 eq) in dioxane (15 mL) was degassed and purged with N₂ 3 times, and then the mixture was stirred at 100° C. for 16 h under N₂ atmosphere. On completion, the reaction mixture was filtered and concentrated under reduced pressure to give a residue. The residue was purified by column chromatography (SiO₂, Petroleum ether/Ethyl acetate=100/1 to 5/1) to give 5-chloro-1-tetrahydropyran-2-yl-4-(4,4,5,5-tetramethyl-1,3,2-dioxaborolan-2-yl)indazole (1.50 g, 3.35 mmol, 81% yield) as yellow oil (I-6-4). ¹H NMR (400 MHz, DMSO-d₆) δ=8.15 (s, 1H), 7.87-7.82 (m, 1H), 7.42 (d, J=8.8 Hz, 1H), 5.88-5.84 (m, 1H), 3.90-3.83 (m, 1H), 3.78-3.70 (m, 1H), 2.41-2.32 (m, 1H), 2.06-2.00 (m, 1H), 1.98-1.91 (m, 1H), 1.79-1.69 (m, 1H), 1.60-1.55 (m, 2H), 1.38 (s, 12H); LCMS: (M+1: 363.1).

Ex. 78 was prepared following General Method D using the above intermediate I-6-4 and intermediate I-5-1 in Step 3.

Ex. 83 and Ex. 84 were prepared from Ex. 78 as shown below:

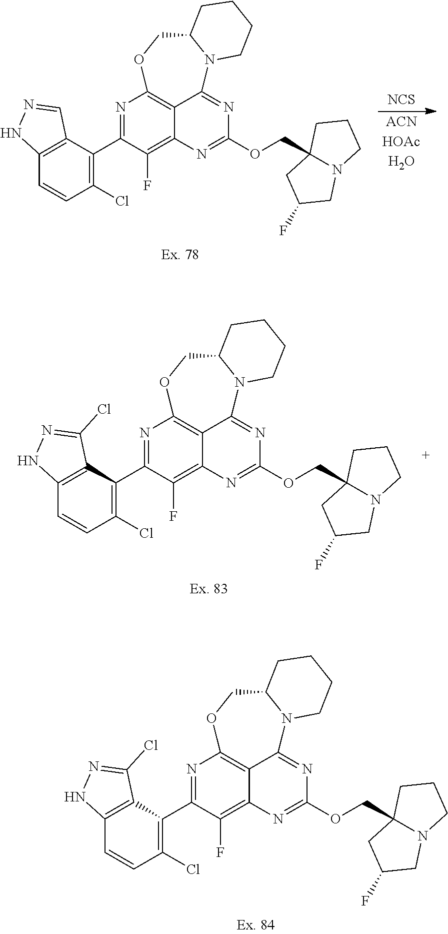

To a solution of (7S)-12-(5-chloro-1H-indazol-4-yl)-13-fluoro-16-[[(2R,8S)-2-fluoro-1,2,3,5,6,7-hexahydropyrrolizin-8-yl]methoxy]-9-oxa-2,11,15,17-tetrazatetracyclo [8.7.1.02,7.014,18]octadeca-1 (17),10 (18),11,13,15-pentaene (10.0 mg, 0.018 mmol, 1 eq, Ex. 78) in Acetonitrile (0.5 mL). H₂O (0.01 mL) and HOAc (0.05 mL) was added NCS (7.05 mg, 0.053 mmol, 3 eq). The mixture was stirred at 40° C. for 16 hours. On completion, the mixture was filtered. to give a residue. The residue was purified by prep-HPLC (column: Phenomenex C18 150*30 mm*5 um; mobile phase: [water(NH₄HCO₃)-ACN]; gradient: 30%-60% B over 15 min) to give (7S)-12-(3,5-dichloro-1H-indazol-4-yl)-13-fluoro-16-[[(2R,8S)-2-fluoro-1,2,3,5,6,6-4hexahydropyrrolizin-8-yl]methoxy]-9-oxa-2,11,15,17-tetrazatetracyclo[8.7.1.02,7.014,18]octadeca-1 (17),10 (18),11,13,15-pentaene (1.50 mg, 0.0024 mmol, 14% yield, 99% purity, Ex. 83) as an off-white solid and (7S)-12-(3,5-dichloro-1H-indazol-4-yl)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02, 7.014,18]octadeca-1 (17),10 (18),11,13,15-pentaene (1.84 mg, 0.003 mmol, 17% yield, 98% purity, Ex. 84) as an off-white solid. Analytical data for Ex. 83: ¹H NMR (400 MHz, DMSO-d₆) δ=13.83-13.23 (m, 1H), 7.74 (d, J=9.2 Hz, 1H), 7.62 (d, J=9.2 Hz, 1H), 5.35-5.20 (m, 1H), 5.12 (d, J=12.8 Hz, 1H), 4.55-4.43 (m, 2H), 4.13 (d, J=10.0 Hz, 1H), 4.00 (d, J=10.4 Hz, 1H), 3.95 (d, J=9.6 Hz, 1H), 3.09 (d, J=7.2 Hz, 2H), 3.01 (s, 2H), 2.86-2.79 (m, 1H), 2.15-2.11 (m, 1H), 2.05 (s, 1H), 2.00 (d, J=6.8 Hz, 1H), 1.92-1.81 (m, 4H), 1.80-1.75 (m, 3H), 1.71-1.64 (m, 1H), 1.54-1.45 (m, 1H); LCMS: (M+1: 602.2). Analytical data for Ex. 84: ¹H NMR (400 MHz, DMSO-d₆) δ=14.05-13.09 (m, 1H), 7.74 (d, J=9.2 Hz, 1H), 7.64-7.59 (m, 1H), 5.35-5.20 (m, 1H), 5.12 (d, J=13.2 Hz, 1H), 4.53-4.45 (m, 2H), 4.13 (d, J=10.4 Hz, 1H), 4.00 (d, J=10.4 Hz, 1H), 3.94 (d, J=10.4 Hz, 1H), 3.11-3.07 (m, 2H), 3.05-3.00 (m, 2H), 2.86-2.79 (m, 1H), 2.15-2.11 (m, 1H), 2.05 (d, J=2.0 Hz, 1H), 2.00 (d, J=7.6 Hz, 1H), 1.92-1.81 (m, 4H), 1.80-1.75 (m, 3H), 1.71-1.65 (m, 1H), 1.54-1.45 (m, 1H); LCMS: (M+1: 602.2).

### Preparation of tert-butyl (3-cyano-7-fluoro-4-(4,4,5,5-tetramethyl-1,3,2-dioxaborolan-2-yl)benzo[b]thiophen-2-yl)carbamate (I-6-5)

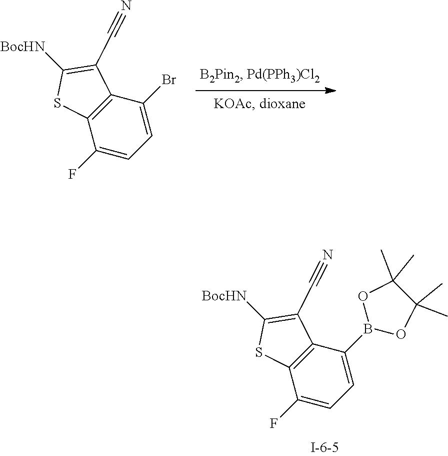 [SMILES: CC(C)(C)OC(=O)Nc1sc2c(F)ccc(Br)c2c1C#N ; CC(C)(C)OC(=O)Nc1sc2c(F)ccc(B3OC(C)(C)C(C)(C)O3)c2c1C#N]

A mixture of tert-butyl N-(4-bromo-3-cyano-7-fluoro-benzothiophen-2-yl)carbamate (1.00 g, 3.00 mmol, 1 eq), 4,4,5,5-tetramethyl-2-(4,4,5,5-tetramethyl-1,3,2-dioxaborolan-2-yl)-1,3,2-dioxaborolane (1.03 g, 4.00 mmol, 1.5 eq), KOAc (793 mg, 8.08 mmol, 3 eq), Pd(PPh₃)₂Cl₂ (189 mg, 0.269 mmol, 0.1 eq) in dioxane (10 mL) was degassed and purged with N₂ 3 times, and then the mixture was stirred at 80° C. for 2 h under N₂ atmosphere. On completion, the mixture was quenched with water (20 mL) and extracted with ethyl acetate (25 mL×3), the combined organic phase was dried over anhydrous sodium sulfate, filtered, and concentrated to give a residue. The residue was purified by column chromatography (SiO₂, Petroleum ether/Ethyl acetate=1:0 to 0:1) to give tert-butyl N-[3-cyano-7-fluoro-4-(4,4,5,5-tetramethyl-1,3,2-dioxaborolan-2-yl)benzothiophen-2-yl]carbamate (1.13 g, 2.00 mmol, 75% yield, 75% purity) as white solid (I-6-5). LCMS: (M+23=441.1).

Ex. 85 was prepared following General Method D, using intermediates I-5-1 and I-6-5 in Step 3.

Ex. 89 was prepared from Intermediate I-10-1 as shown below:

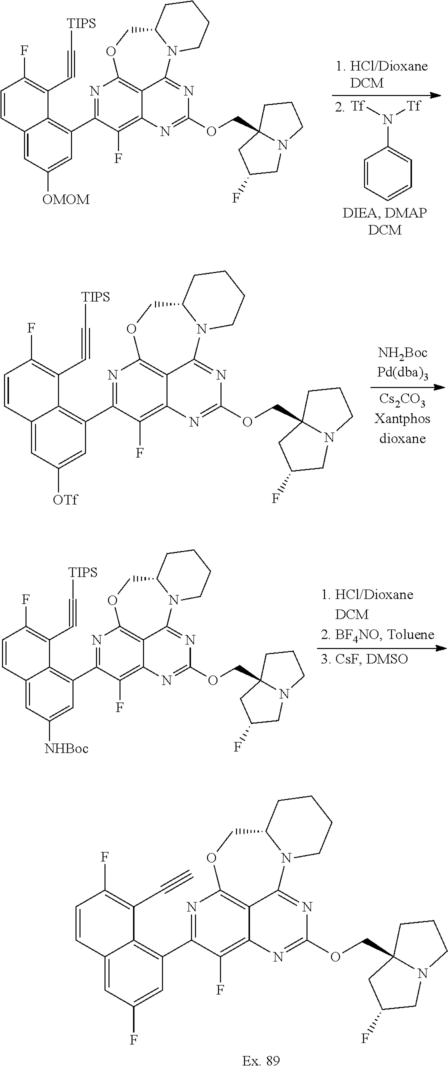

Step 1. To a solution of 2-[2-fluoro-8-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02, 7.014,18]octadeca-1 (17),10 (18),11,13,15-pentaen-12-yl]-6-(methoxymethoxy)-1-naphthyl]ethynyl-triisopropyl-silane (730 mg, 0.910 mmol, 1.00 eq) in DCM (7 mL) was added HCl/dioxane (4 M, 1.4 mL, 6.00 eq). The mixture was stirred at 25° C. for 10 min. On completion, the reaction mixture was concentrated to give 6-fluoro-4-[(7S)-13-fluoro-16-[[(2R,8S)-2-fluoro-1,2,3,5,6,7-hexahydropyrrolizin-8-yl]meth oxy]-9-oxa-2,11,15,17-tetrazatetracyclo [8.7.1.02,7.014,18]octadeca-1(17), 10 (18),11,13,15-pentaen-12-yl]-5-(2-triisopropylsilylethynyl)naphthalen-2-ol (770 mg, 0.792 mmol, 87% yield, 78% purity) as yellow solid. LCMS: (M+1=758.4).

To a solution of 6-fluoro-4-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02, 7.014,18]octadeca-1 (17),10 (18),11,13,15-pentaen-12-yl]-5-(2-triisopropylsilylethynyl) naphthalen-2-ol (400 mg, 0.528 mmol, 1.00 eq) in DCM (5 mL) was added DMAP (12.8 mg, 0.106 mmol, 0.20 eq), DIPEA (136 mg, 1 mmol, 0.183 mL, 2.00 eq) and 1,1,1-trifluoro-N-phenyl-N-(trifluoromethylsulfonyl)methanesulfonamide (282 mg, 0.792 mmol, 1.50 eq). The mixture was stirred at 25° C. for 2 h. On completion, the mixture was quenched with water (20 mL) and extracted with DCM (15 mL×3), the combined organic phase was dried over anhydrous sodium sulfate, filtered, and concentrated to give a residue. The residue was purified by column chromatography (SiO₂, Petroleum ether/THF=1:0 to 0:1) to give [6-fluoro-4-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo [8.7.1.02, 7.014,18]octadeca-1 (17),10 (18),11,13,15-pentaen-12-yl]-5-(2-triisopropylsilylethynyl)-2-naphthyl]trifluoromethanesulfonate (390 mg, 0.438 mmol, 83% yield) as yellow oil. LCMS: (M+1=890.4).

Step 2. A mixture of [6-fluoro-4-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo [8.7.1.02,7.014,18]octadeca-1 (17),10 (18),11,13,15-pentaen-12-yl]-5-(2-triisopropylsilylethynyl)-2-naphthyl]trifluoromethanesulfonate (340 mg, 0.382 mmol, 1.00 eq), Pd₂(dba)₃ (34.9 mg, 0.038 mmol, 0.10 eq), Cs₂CO₃ (348 mg, 1.07 mmol, 3.00 eq), Xantphos (66.3 mg, 0.114 mmol, 0.30 eq) and tert-butyl carbamate (107 mg, 0.917 mmol, 2.00 eq) in dioxane (3 mL) was degassed and purged with N₂ 3 times, and then the mixture was stirred at 100° C. for 2 h under N₂ atmosphere. On completion, the mixture was quenched with water (20 mL) and extracted with ethyl acetate (25 mL×3), the combined organic phase was dried over anhydrous sodium sulfate, filtered, and concentrated to give a residue. The residue was purified by column chromatography (SiO₂, Petroleum ether/THF=1:0 to 0:1) to give tert-butyl N-[6-fluoro-4-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaen-12-yl]-5-(2-triisopropylsilylethynyl)-2-naphthyl]carbamate (280 mg, 0.327 mmol, 86% yield) as yellow oil. LCMS: (M+1=857.5).

Step 3. To a solution of tert-butyl N-[6-fluoro-4-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaen-12-yl]-5-(2-triisopropylsilylethynyl)-2-naphthyl]carbamate (260 mg, 0.303 mmol, 1.00 eq) in DCM (2 mL) was added HCl/dioxane (4 M, 0.4 mL, 5.00 eq). The mixture was stirred at 25° C. for 1h. On completion, the reaction mixture was concentrated to give 6-fluoro-4-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaen-12-yl]-5-(2-triisopropylsilylethynyl)naphthalen-2-amine (200 mg, 0.264 mmol, 87% yield) as yellow solid.

To a solution of 6-fluoro-4-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaen-12-yl]-5-(2-triisopropylsilylethynyl)naphthalen-2-amine (160 mg, 0.211 mmol, 1.00 eq) in Toluene (2 mL) was added nitridooxonium; tetrafluoroborate (29.6 mg, 0.253 mmol, 1.20 eq). The mixture was stirred at 25° C. for 16 h, and then stirred at 100° C. for 1 h. On completion, the mixture was quenched with water (20 mL) and extracted with ethyl acetate (15 ml×3), the combined organic phase was dried over anhydrous sodium sulfate, filtered and concentrated to give 2-[2,6-difluoro-8-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014.18]octadeca-1 (17),10 (18),11,13,15-pentaen-12-yl]-1-naphthyl]ethynyl-triisopropyl-silane (100 mg, 0.131 mmol, 62% yield) as brown solid. LCMS: (M+1=760.4).

To a solution of 2-[2,6-difluoro-8-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaen-12-yl]-1-naphthyl]ethynyl-triisopropyl-silane (100 mg, 0.132 mmol, 1.00 eq) in DMSO (0.5 mL) was added CsF (239 mg, 1.58 mmol, 12.0 eq). The mixture was stirred at 25° C. for 1h. On completion, the mixture was filtrated. The residue was purified by Prep-HPLC (column: Phenomenex luna C18 150*25 mm*10 um; mobile phase: [water(FA)-ACN]; gradient: 20%-50% B over 8 min) to give (7S)-12-(8-ethynyl-3,7-difluoro-1-naphthyl)-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.02,7.014,18]octadeca-1 (17),10 (18),11,13,15-pentaene (30.77 mg, 0.046 mmol, 35.16% yield, 97.69% purity, FA) as yellow solid (Ex. 89). ¹H NMR (400 MHz, DMSO-d₆) δ=8.23-8.15 (m, 1H), 8.04 (dd, J=2.8, 9.6 Hz, 1H), 7.66 (s, 2H), 5.36-5.19 (m, 1H), 4.49 (d, J=13.6 Hz, 2H), 4.26-4.18 (m, 1H), 4.11 (s, 1H), 4.01-3.91 (m, 2H), 3.13-3.07 (m, 2H), 3.06-2.98 (m, 2H), 2.85-2.79 (m, 1H), 2.14-2.05 (m, 2H), 2.00-1.68 (m, 11H); LCMS: (M+1=604.2).

Ex. 93 was prepared following General Method D, using Intermediate I-2-23 in Step 1, and using XPhos Pd G4 (instead of cataCXium® A Pd G3) in Step 3.

### Preparation of (9S)-9-ethyl-4-fluoro-5-[7-fluoro-3-(methoxymethoxy)-8-{[tri(propan-2-yl)silyl]ethynyl}naphthalen-1-yl]-10-methyl-2-(methylsulfanyl)-9,10-dihydro-8H-7-oxa-1,3,6,10-tetraazacyclohepta[1,2,3-de]naphthalene (I-7-96)

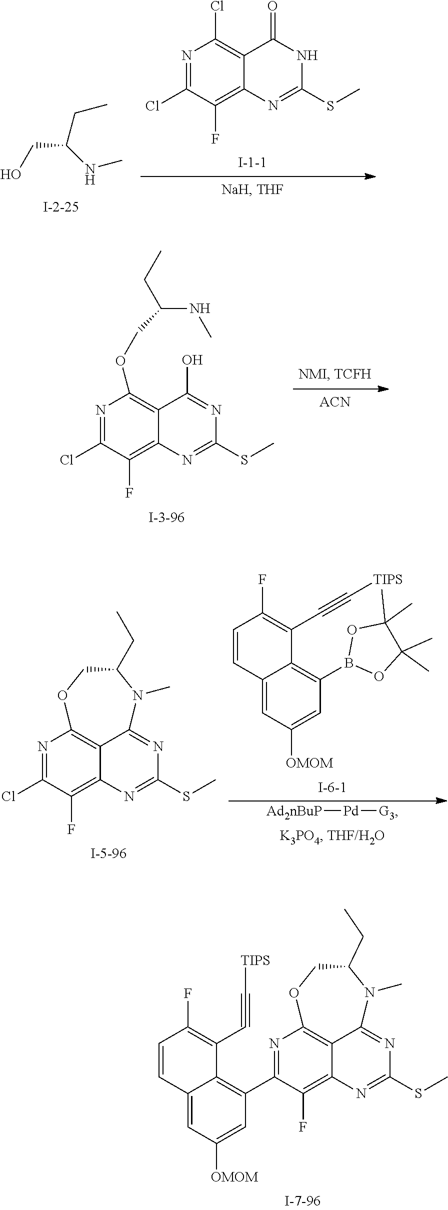

Step 1. To a solution of (2S)-2-(methylamino)butan-1-ol (500 mg, 4.85 mmol, 1 eq) in THF (20 mL) was added NaH (484 mg, 12.1 mmol, 60% purity, 2.5 eq) and 5,7-dichloro-8-fluoro-2-methylsulfanyl-3H-pyrido[4,3-d]pyrimidin-4-one (1.36 g, 4.85 mmol, 1 eq). The mixture was stirred at 0° C. for 2 hours. On completion, the mixture was quenched with water (20 mL) and extracted with ethyl acetate (20 mL×3), the combined organic phase was dried over anhydrous sodium sulfate, filtered, and concentrated to give a residue. The residue was purified by column chromatography (SiO₂, DCM/MeOH=1/0 to 0/1) to give 7-chloro-8-fluoro-5-[(2S)-2-(methylamino)butoxy]-2-methylsulfanyl-pyrido [4,3-d]pyrimidin-4-ol (1.07 g, 3.09 mmol, 63% yield) as yellow solid. ¹H NMR (400 MHz, DMSO-d₆) δ=9.32-8.97 (m, 1H), 4.82-4.40 (m, 2H), 3.55-3.49 (m, 1H), 3.42 (br s, 1H), 2.65-2.55 (m, 3H), 2.50 (s, 3H), 1.90-1.64 (m, 2H), 1.05 (t, J=7.2 Hz, 3H).

Step 2. To a solution of 7-chloro-8-fluoro-5-[(2S)-2-(methylamino)butoxy]-2-methylsulfanyl-pyrido[4,3-d]pyrimidin-4-ol (500 mg, 1.44 mmol, 1 eq) in Acetonitrile (40 mL) was added NMI (1.42 g, 17.3 mmol, 12 eq) and TCFH (1.21 g, 4.33 mmol, 3 eq). The mixture was stirred at 25° C. for 14 hours. On completion, the reaction mixture was partitioned between ethyl acetate (20 mL×3) and water (20 mL), the combined organic phase was dried over anhydrous sodium sulfate, filtered, and concentrated to give a residue. The residue was purified by column chromatography (SiO₂, Petroleum ether/Ethyl acetate=1:0 to 0:1) to give (12S)-7-chloro-12-ethyl-6-fluoro-13-methyl-3-methylsulfanyl-10-oxa-2,4,8,13-tetrazatricyclo[7.4.1.0^{5,14}]tetradeca-1,3,5,7,9 (14)-pentaene (470 mg, 1.43 mmol, 99% yield) as yellow solid. ¹H NMR (400 MHz, CDCl₃) δ=4.64 (dd, J=4.8, 13.2 Hz, 1H), 4.22 (d, J=13.2 Hz, 1H), 3.58 (dt, J=4.8, 7.6 Hz, 1H), 3.36 (s, 3H), 2.54 (s, 3H), 1.70 (quin, J=7.6 Hz, 2H), 1.06-0.96 (m, 3H).

Step 3. A mixture of (12S)-7-chloro-12-ethyl-6-fluoro-13-methyl-3-methylsulfanyl-10-oxa-2,4,8,13-tetrazatricyclo[7.4.1.0^{5,14}]tetradeca-1,3,5,7,9 (14)-pentaene (430 mg, 1.31 mmol, 1 eq), 2-[2-fluoro-6-(methoxymethoxy)-8-(4,4,5,5-tetramethyl-1,3,2-dioxaborolan-2-yl)-1-naphthyl]ethynyl-triisopropyl-silane (737 mg, 1.44 mmol, 1.1 eq), [2-(2-aminophenyl)phenyl]palladium(1+); bis(1-adamantyl)-butyl-phosphane; methanesulfonate (95.2 mg, 0.130 mmol, 0.1 eq), K₃PO₄ (832 mg, 3.92 mmol, 3 eq) in THF (20 mL) and H₂O (2 mL) was degassed and purged with N₂ 3 times, and then the mixture was stirred at 80° C. for 12 hours under N₂ atmosphere. On completion, the reaction mixture was partitioned between ethyl acetate (20 mL×3) and water (20 mL), the combined organic phase was dried over anhydrous sodium sulfate, filtered, and concentrated to give a residue. The residue was purified by column chromatography (SiO₂, Petroleum ether/Ethyl acetate=1:0 to 0:1) to give 2-[8-[(12S)-12-ethyl-6-fluoro-13-methyl-3-methylsulfanyl-10-oxa-2,4,8,13-tetrazatricyclo[7.4.1.0^{5,14}]tetradeca-1,3,5,7,9 (14)-pentaen-7-yl]-2-fluoro-6-(methoxymethoxy)-1-naphthyl]ethynyl-triisopropyl-silane (410 mg, 0.603 mmol, 46% yield) as a yellow solid (I-7-96). ¹H NMR (400 MHz, CDCl₃) δ=7.67 (br d, J=3.2 Hz, 1H), 7.40-7.37 (m, 2H), 7.23 (d, J=2.4 Hz, 1H), 5.26-5.21 (m, 2H), 4.62 (br d, J=4.4 Hz, 2H), 4.27-4.21 (m, 2H), 4.20-4.18 (m, 1H), 3.56 (dt, J=3.2, 7.6 Hz, 3H), 1.83 (br s, 3H), 1.72-1.68 (m, 3H), 0.85-0.82 (m, 21H), 0.63-0.58 (m, 3H).

Ex. 96 was prepared following General Method F (Steps 1-3), followed by General Method D (Steps 4-6), using above intermediate I-7-96.

### Preparation of ((2R,7aS)-2-((tert-butyldiphenylsilyl)oxy)tetrahydro-1H-pyrrolizin-7a(5H)-yl)methanol (I-9-3)

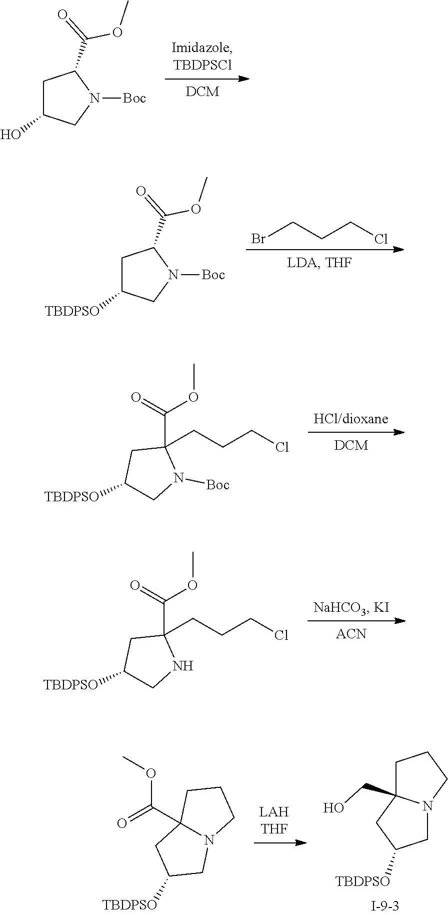

Step 1. To a solution of 01-tert-butyl 02-methyl (2R,4R)-4-hydroxypyrrolidine-1,2-dicarboxylate (12.0 g, 48.9 mmol, 1 eq) in DCM (120 mL) was added Imidazole (6.66 g, 97.9 mmol, 2 eq) and TBDPSCl (20.2 g, 73.4 mmol, 1.5 eq). The mixture was stirred at 25° C. for 1 h. On completion, the mixture was diluted with dilute citric acid (300 mL) and extracted with DCM (120 mL×3). The combined organic phase was washed with water (200 mL×2) and dried over Na₂SO₄, filtered and the filtrate was concentrated to give a residue. The residue was purified by column chromatography (SiO₂, PE:THF=1:1) to give O1-tert-butyl O2-methyl (2R,4R)-4-[tert-butyl(diphenyl)silyl]oxypyrrolidine-1,2-dicarboxylate (23.2 g, crude) as a white solid. LCMS: (M+23: 506.1).

Step 2. The mixture of O1-tert-butyl O2-methyl (2R,4R)-4-[tert-butyl(diphenyl)silyl]oxypyrrolidine-1,2-dicarboxylate (22.2 g, 45.9 mmol, 1 eq) in THE (220 mL) was added LDA (2 M, 30 mL, 1.3 eq) at −70° C., then the mixture was stirred at −70° C. for 1 h, then 1-bromo-3-chloro-propane (36.1 g, 229 mmol, 5 eq) was added at −70° C. The mixture was stirred at 20° C. for 15 h. On completion, the mixture was quenched by NH₄Cl (600 mL) at 0° C. and extracted with ethyl acetate (300 mL×3). The combined organic phase was washed with water (300 mL*2) and dried over Na₂SO₄, filtered and the filtrate was concentrated to give a residue. The residue was purified by column chromatography (SiO₂, PE:THF=10:1) to give O1-tert-butyl O2-methyl (4R)-4-[tert-butyl(diphenyl) silyl]oxy-2-(3-chloropropyl)pyrrolidine-1,2-dicarboxylate (23.8 g, crude) as a colorless oil. LCMS: (M+23: 582.2).

Step 3. To a solution of 01-tert-butyl 02-methyl (4R)-4-[tert-butyl(diphenyl)silyl]oxy-2-(3-chloropropyl)pyrrolidine-1,2-dicarboxylate (12.0 g, 21.4 mmol, 1 eq) in DCM (120 mL) was added HCl/dioxane (4 M, 56 mL, 10.4 eq). The mixture was stirred at 25° C. for 1 h. On completion, the mixture was concentrated to give methyl (4R)-4-[tert-butyl(diphenyl)silyl]oxy-2-(3-chloropropyl)pyrrolidine-2-carboxylate (11.8 g, crude, HCl) as a yellow solid. LCMS: (M+1: 460.1).

Step 4. To a solution of methyl (4R)-4-[tert-butyl(diphenyl)silyl]oxy-2-(3-chloropropyl)pyrrolidine-2-carboxylate (22.8 g, 46.0 mmol, 1 eq, HCl) in Acetonitrile (220 mL) was added NaHCO₃ (19.3 g, 230 mmol, 5 eq) and KI (763 mg, 4.60 mmol, 0.1 eq). The mixture was stirred at 50° C. for 16 h. On completion, the mixture was filtered and concentrated to give a residue. The residue was purified by prep-HPLC (neutral condition; about 20 grams of sample dissolved in 60 ml of ACN) to give methyl (2R)-2-[tert-butyl(diphenyl)silyl]oxy-1,2,3,5,6,7-hexahydropyrrolizine-8-carboxylate (9.00 g, crude) as a yellow oil. LCMS: (M+1: 424.1).

Step 5. To a solution of methyl (2R)-2-[tert-butyl(diphenyl)silyl]oxy-1,2,3,5,6,7-hexahydropyrrolizine-8-carboxylate (4.23 g, 9.99 mmol, 1 eq) in THF (45 mL) was added LAH (2.5 M, 12 mL, 3 eq) at −40° C. The mixture was stirred at −40° C. for 1 h. On completion, the mixture was quenched with water (2 mL), 15% NaOH (2 mL) and water (4 mL). The combined organic phase was dried over Na₂SO₄, filtered and the filtrate was concentrated to give a residue. The residue was purified by column chromatography (SiO₂, DCM:MeOH=10:1) to give [(2R,8S)-2-[tert-butyl(diphenyl) silyl]oxy-1,2,3,5,6,7-hexahydropyrrolizin-8-yl]methanol (4.01 g, crude) as a yellow oil (I-9-3). LCMS: (M+1: 396.1).

Ex. 97 was prepared following General Method D, using above intermediate I-9-3 in Step 5.

Ex. 99 was prepared following General Method F using Intermediate I-6-33 in Step 3, followed by General Method D (Steps 4-6).

### Preparation of 5-ethyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-9,10-dihydro-8H-7-oxa-1,3,6,10-tetraazacyclohepta[1,2,3-de]naphthalen-5-yl]naphthalen-2-ol (Ex. 100)

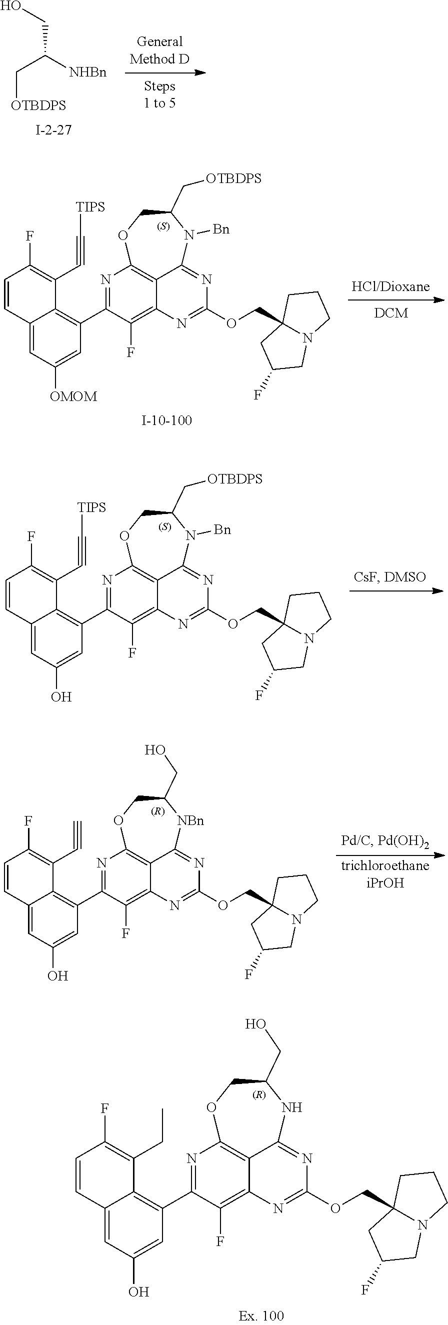

Step 1. Intermediate I-10-100 was prepared following General Method D (steps 1-5), using intermediate I-2-27 in Step 1.

Step 2. To a solution of 2-[8-[(12R)-13-benzyl-12-[[tert-butyl(diphenyl)silyl]oxymethyl]-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.0⁵⁴]tetradeca-1,3,5,7,9 (14)-pentaen-7-yl]-2-fluoro-6-(methoxymethoxy)-1-naphthyl]ethynyl-triisopropyl-silane (63.0 mg, 0.057 mmol, 1 eq, I-10-100) in DCM (1 mL) was added HCl/dioxane (4 M, 0.142 mL, 10 eq). The mixture was stirred at 25° C. for 1 h. On completion, the mixture was concentrated to give 4-[(12R)-13-benzyl-12-[[tert-butyl(diphenyl)silyl]oxymethyl]-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.0^{5,14}]tetradeca-1,3,5,7,9 (14)-pentaen-7-yl]-6-fluoro-5-(2-triisopropylsilylethynyl)naphthalen-2-ol (61 mg, crude) as a yellow solid. LCMS: (M+1: 1062.3).

Step 3. To a solution of 4-[(12R)-13-benzyl-12-[[tert-butyl(diphenyl)silyl]oxymethyl]-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.0⁵,4]tetradeca-1,3,5,7,9 (14)-pentaen-7-yl]-6-fluoro-5-(2-triisopropylsilylethynyl)naphthalen-2-ol (61.0 mg, 0.057 mmol, 1 eq) in DMSO (1 mL) was added CsF (61.0 mg, 0.401 mmol, 7 eq). The mixture was stirred at 25° C. for 2 h. On completion, the mixture was quenched with sat. NaCl (5 mL) at 0° C. and extracted with 2-MeTHF (2 mL×3), the combined organic phase was dried over anhydrous Na₂SO₄, filtered and the filtrate was concentrated to give a residue. The residue was purified by column chromatography (SiO₂, DCM:MeOH=10:1) to give 4-[(12S)-13-benzyl-6-fluoro-3-[[(2R,8S)-2-fluoro-1,2,3,5,6,7-hexahydropyrrolizin-8-yl]methoxy]-12-(hydroxymethyl)-10-oxa-2,4,8,13-tetrazatricyclo[7.4.1.0^{5,14}]tetradeca-1,3,5,7,9 (14)-pentaen-7-yl]-5-ethynyl-6-fluoro-naphthalen-2-ol (4.00 mg, crude) as a yellow solid. LCMS: (M+1: 668.1).

Step 4. To a solution of 4-[(12S)-13-benzyl-6-fluoro-3-[[(2R,8S)-2-fluoro-1,2,3,5,6,7-hexahydropyrrolizin-8-yl]methoxy]-12-(hydroxymethyl)-10-oxa-2,4,8,13-tetrazatricyclo[7.4.1.0^{5,14}]tetradeca-1,3,5,7,9 (14)-pentaen-7-yl]-5-ethynyl-6-fluoro-naphthalen-2-ol (4.00 mg, 0.0060 mmol, 1 eq), 1,1,2-trichloroethane (0.879 mg, 0.0066 mmol, 1.1 eq) in i-PrOH (1 mL) was added Pd/C (1.00 mg, 0.0009 mmol, 10% purity, 0.016 eq) and Pd(OH)₂ (1.00 mg, 0.0014 mmol, 20% purity, 0.238 eq). The mixture was stirred at 25° C. for 16 h. On completion, the mixture was filtered and concentrated to give a residue. The residue was purified by prep-HPLC (neutral condition; column: Waters Xbridge 150*25 mm*5 um; mobile phase: [water (NH₄HCO₃)-ACN]; gradient: 24%-54% B over 10 min) to give 5-ethyl-6-fluoro-4-[(12S)-6-fluoro-3-[[(2R,8S)-2-fluoro-1,2,3,5,6,7-hexahydropyrrolizin-8-yl]methoxy]-12-(hydroxymethyl)-10-oxa-2,4,8,13-tetrazatricyclo[7.4.1.0^{5,14}]tetradeca-1,3,5,7,9 (14)-pentaen-7-yl]naphthalen-2-ol (2.09 mg, 0.0036 mmol, 59.98% yield) as an off-white solid (Ex. 100). ¹H NMR (400 MHz, MeOD-d₄) δ=7.71-7.60 (m, 1H), 7.32-7.18 (m, 2H), 7.10-7.03 (m, 1H), 5.40-5.19 (m, 1H), 4.63-4.51 (m, 2H), 4.32-4.18 (m, 2H), 3.97-3.87 (m, 1H), 3.86-3.70 (m, 2H), 3.07-2.96 (m, 1H), 2.64-2.42 (m, 1H), 2.40-2.16 (m, 3H), 2.15-2.06 (m, 1H), 2.05-1.82 (m, 3H), 1.31 (br d, J=16.8 Hz, 3H), 0.95-0.80 (m, 3H); LCMS: (M+1: 582.2).

### Preparation of ((2S,7aS)-2-((tert-butyldiphenylsilyl)oxy)tetrahydro-1H-pyrrolizin-7a(5H)-yl)methanol (I-9-4)

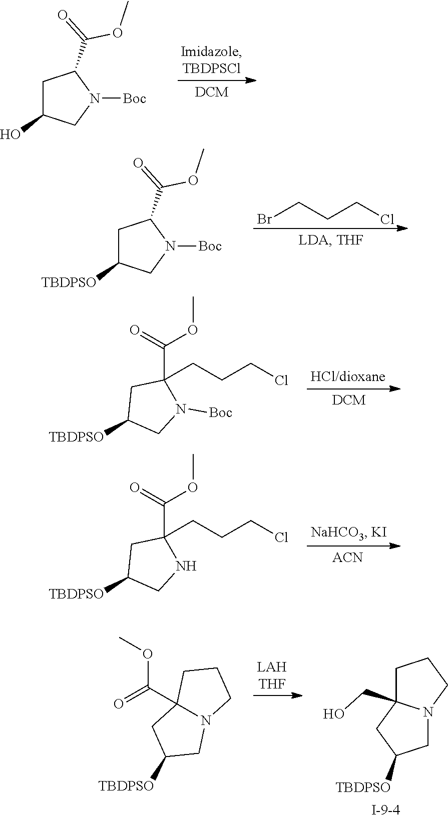

Intermediate I-9-4 was prepared following the same procedure as the preparation of I-9-3.

Ex. 101 was prepared following General Method D, using above intermediate I-9-4 in Step 5.

Ex. 104 was prepared following General Method D, using intermediate I-3-104 (from the synthesis of Ex. 25) in Step 2.

Ex. 105, Ex. 106, and Ex. 107 were synthesized using Intermediate I-11-29 (from the synthesis of Ex. 29) as shown below.

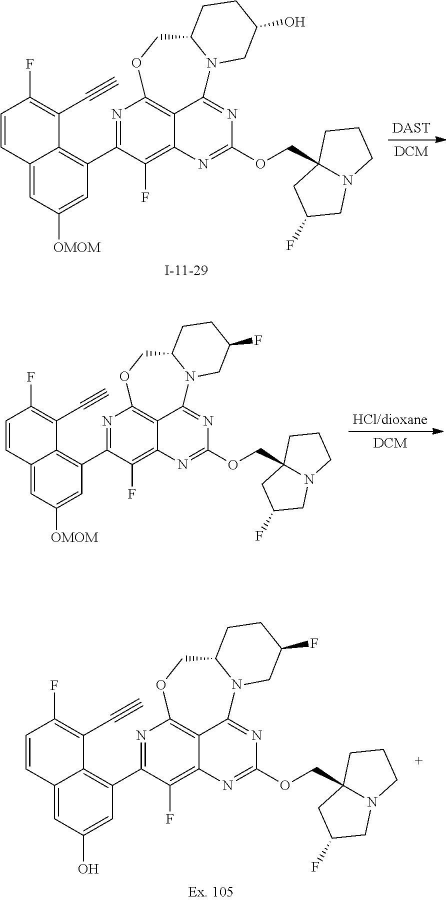

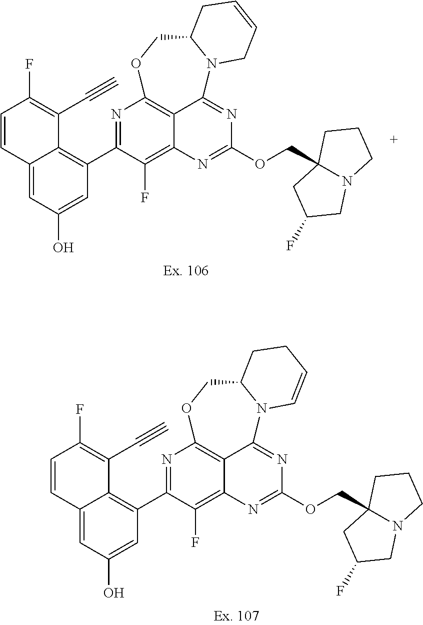 [SMILES: C#Cc1c(F)ccc2cc(O)cc(-c3nc4c5c(nc(OC[C@@]67CCCN6C[C@H](F)C7)nc5c3F)N3CC=CC[C@H]3CO4)c12 ; C#Cc1c(F)ccc2cc(O)cc(-c3nc4c5c(nc(OC[C@@]67CCCN6C[C@H](F)C7)nc5c3F)N3C=CCC[C@H]3CO4)c12]

Step 1. To a solution of (4S,7S)-12-[8-ethynyl-7-fluoro-3-(methoxymethoxy)-1-naphthyl]-13-fluoro-16-[[(2R,8S)-2-fluoro-1,2,3,5,6,7-hexahydropyrrolizin-8-yl]methoxy]-9-oxa-2,11,15,17-tetrazatetracyclo[8.7.1.0^{2,7}.0^{14,18}]octadeca-1 (17),10 (18),11,13,15-pentaen-4-ol (20.0 mg, 0.030 mmol, 1 eq, I-11-29) in DCM (0.5 ml) was added DAST (19.4 mg, 0.12 mmol, 4 eq). The mixture was stirred at 25° C. for 4 h. On completion, the reaction mixture was quenched by addition H₂O (0.2 mL) at 25° C., and then diluted with H₂O (5 mL) and extracted with DCM (3×5 mL). The combined organic layers were washed with brine (5 mL), dried over Na₂SO₄, filtered and concentrated under reduced pressure to give (4S,7S)-12-[8-ethynyl-7-fluoro-3-(methoxymethoxy)-1-naphthyl]-4,13-difluoro-16-[[(2R,8S)-2-fluoro-1,2,3,5,6,7-hexahydropyrrolizin-8-yl]methoxy]-9-oxa-2,11,15,17-tetrazatetracyclo[8.7.1.0^{2,7}.0^{14,18}]octadeca-1 (17),10 (18),11,13,15-pentaene (19.0 mg, 0.029 mmol, 95% yield) as yellow oil. LCMS: (M+1: 644.1).

Step 2. To a solution of (4R,7S)-12-[8-ethynyl-7-fluoro-3-(methoxymethoxy)-1-naphthyl]-4,13-difluoro-16-[[(2R,8S)-2-fluoro-1,2,3,5,6,7-hexahydropyrrolizin-8-yl]methoxy]-9-oxa-2,11,15,17-tetrazatetracyclo[8.7.1.0^{2,7}.0^{14,18}]octadeca-1 (17),10 (18),11,13,15-pentaene (19.0 mg, 0.029 mmol, 1 eq) in DCM (0.5 mL) was added HCl/dioxane (4 M, 0.008 mL, 1.1 eq). The mixture was stirred at 25° C. for 0.5 h. On completion, the reaction mixture was filtered and concentrated under reduced pressure to give a residue. The residue was purified by prep-HPLC (column: Phenomenex luna C18 150*25 mm*10 um; mobile phase: [water(FA)-ACN]; gradient: 14%-44% B over 10 min) and SFC (column: DAICEL CHIRALCEL OX (250 mm*30 mm, 10 um); mobile phase: [CO₂-MeOH]; B %: 40%, isocratic elution mode) to give 4-[(4R,7S)-4,13-difluoro-16-[[(2R,8S)-2-fluoro-1,2,3,5,6,7-hexahydropyrrolizin-8-yl]methoxy]-9-oxa-2,11,15,17-tetrazatetracyclo[8.7.1.0^{2,7}.0^{14,18}]octadeca-1 (17),10 (18),11,13,15-pentaen-12-yl]-5-ethynyl-6-fluoro-naphthalen-2-ol (1.47 mg, 0.0022 mmol, 7% yield, 91% purity) as a brown solid (Ex. 105), 5-ethynyl-6-fluoro-4-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo [8.7.1.0^{2,7}.0^{14,18}]octadeca-1 (17),4,10 (18),11,13,15-hexaen-12-yl]naphthalen-2-ol (2 mg, 0.0031 mmol, 10.29% yield, 93% purity) as a brown solid (Ex. 106) and 5-ethynyl-6-fluoro-4-[(7S)-13-fluoro-16-[[(2R,8S)-2-fluoro-1,2,3,5,6,7-hexahydropyrrolizin-8-yl]methoxy]-9-oxa-2,11,15,17-tetrazatetracyclo [8.7.1.0^{2,7}.0^{14,18}]octadeca-1 (17),3,10 (18),11,13,15-hexaen-12-yl]naphthalen-2-ol (1.92 mg, 0.0029 mmol, 9.67% yield, 91% purity) as a brown solid (Ex. 107). Analytical data for Ex. 105: ¹H NMR (400 MHz, DMSO-d₆) δ=10.18-10.10 (m, 1H), 7.99-7.91 (m, 1H), 7.49-7.43 (m, 1H), 7.37 (d, J=2.4 Hz, 1H), 7.21-7.08 (m, 1H), 5.36-5.10 (m, 2H), 4.59-4.42 (m, 2H), 4.17-3.97 (m, 4H), 3.10 (d, J=9.6 Hz, 2H), 3.03 (s, 1H), 2.88-2.80 (m, 1H), 2.37-2.23 (m, 2H), 2.15-2.06 (m, 2H), 2.02-1.93 (m, 2H), 1.88-1.82 (m, 2H), 1.81-1.76 (m, 2H), 1.47 (s, 2H), 1.40 (s, 2H), 1.23 (s, 2H); LCMS: (M+1: 620.1). Analytical data for Ex. 106: ¹H NMR (400 MHz, DMSO-d₆) δ=10.22-10.07 (m, 1H), 8.00-7.92 (m, 1H), 7.50-7.42 (m, 1H), 7.37 (d, J=2.0 Hz, 1H), 7.21-7.09 (m, 1H), 5.98-5.78 (m, 2H), 5.38-5.19 (m, 1H), 5.09-4.97 (m, 1H), 4.64-4.51 (m, 2H), 4.13-4.11 (m, 1H), 4.02-3.99 (m, 1H), 3.10 (d, J=9.2 Hz, 2H), 3.02 (s, 1H), 2.87-2.79 (m, 1H), 2.34-2.27 (m, 1H), 2.14-2.04 (m, 2H), 2.02-1.97 (m, 1H), 1.86-1.81 (m, 1H), 1.80-1.70 (m, 2H), 1.47 (s, 2H), 1.23 (s, 2H); LCMS: (M+1: 600.3). Analytical data for Ex. 107: ¹H NMR (400 MHz, DMSO-d₆) δ=10.28-9.96 (m, 1H), 7.99-7.94 (m, 1H), 7.49-7.43 (m, 1H), 7.37 (s, 1H), 7.22-7.10 (m, 1H), 5.44 (d, J=3.2 Hz, 1H), 5.37-5.18 (m, 1H), 4.69-4.47 (m, 2H), 4.16-3.99 (m, 4H), 3.09 (d, J=8.4 Hz, 211), 3.02 (s, 111), 2.86-2.80 (m, 1H), 2.29-2.20 (m, 1H), 2.13 (d, J=1.2 Hz, 1H), 2.03 (d, J=15.2 Hz, 2H), 1.86-1.80 (m, 2H), 1.79-1.76 (m, 1H), 1.47 (s, 2H), 1.25-1.22 (m, 2H); LCMS: (M+1: 600.3).

### Preparation of 4-(10-ethyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl)-5-ethynyl-6-fluoronaphthalen-2-ol (Ex. 108)

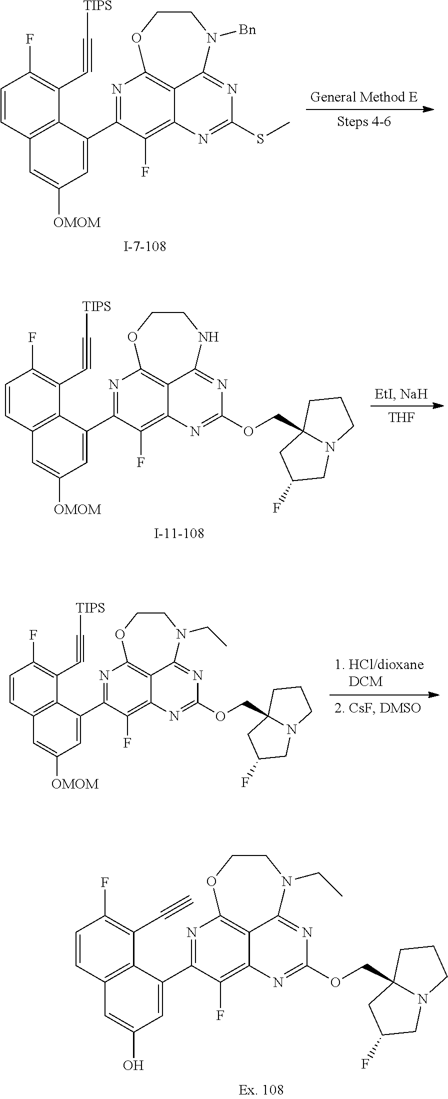

I-7-108 was synthesized following General Method F, using intermediate I-2-29.

Step 1. Intermediate I-7-108 was converted to I-11-108 following General Method E, Step 4 to Step 6.

Step 2. To a solution of 2-[2-fluoro-8-[6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.0^{5,14}]tetradeca-1,3,5,7,9 (14)-pentaen-7-yl]-6-(methoxymethoxy)-1-naphthyl]ethynyl-triisopropyl-silane (100 mg, 0.133 mmol, 1 eq) in THF (1 mL) was added iodoethane (62.5 mg, 0.401 mmol, 3 eq) and NaH (10.7 mg, 0.267 mmol, 60% purity, 2 eq) at 0° C. The mixture was stirred at 25° C. for 1 hour. On completion, the mixture was quenched with water (10 mL) and extracted with ethyl acetate (10 mL*3), the combined organic phase was dried over anhydrous sodium sulfate, filtered, and concentrated to give a residue. The residue was purified by column chromatography (SiO₂, Petroleum ether/Ethyl acetate=1:0 to 0:1) to give 2-[8-[13-ethyl-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.0^{5,14}]tetradeca-1,3,5,7,9 (14)-pentaen-7-yl]-2-fluoro-6-(methoxymethoxy)-1-naphthyl]ethynyl-triisopropyl-silane (20.0 mg, 0.026 mmol, 19% yield) as yellow solid. ¹H NMR (400 MHz, CDCl₃) δ=7.85-7.66 (m, 1H), 7.54-7.33 (m, 1H), 6.99 (s, 2H), 5.44-5.18 (m, 2H), 5.02 (s, 1H), 4.66-4.40 (m, 1H), 4.17-3.70 (m, 3H), 3.50 (s, 2H), 2.28 (s, 3H), 1.44 (s, 18H), 1.38-1.24 (m, 3H), 1.10-0.96 (m, 2H), 0.92 (t, J=6.4 Hz, 10H), 0.73-0.60 (m, 3H); LCMS: (M+1: 776.3).

Step 3. To a solution of 2-[8-[13-ethyl-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.0^{5,14}]tetradeca-1,3,5,7,9 (14)-pentaen-7-yl]-2-fluoro-6-(methoxymethoxy)-1-naphthyl]ethynyl-triisopropyl-silane (45.0 mg, 0.058 mmol, 1 eq) in DCM (0.5 mL) was added HCl/dioxane (4 M, 0.015 mL, 1 eq). The mixture was stirred at 25° C. for 0.5 hours. On completion, the mixture was concentrated to give 4-[13-ethyl-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.0^{5,14}]tetradeca-1,3,5,7,9 (14)-pentaen-7-yl]-6-fluoro-5-(2-triisopropylsilylethynyl)naphthalen-2-ol (40.0 mg, 0.055 mmol, 94% yield) as yellow oil. LCMS: (M+1: 732.1).

To a solution of 4-[13-ethyl-6-fluoro-3-[[(2R,8S)-2-fluoro-1,2,3,5,6,7-hexahydropyrrolizin-8-yl]methoxy]-10-oxa-2,4,8,13-tetrazatricyclo[7.4.1.0^{5,14}]tetradeca-1,3,5,7,9 (14)-pentaen-7-yl]-6-fluoro-5-(2-triisopropylsilylethynyl)naphthalen-2-ol (35.0 mg, 0.048 mmol, 1 eq) in DMSO (0.5 mL) was added CsF (43.5 mg, 0.286 mmol, 6 eq). The mixture was stirred at 25° C. for 0.5 hours. On completion, the crude product was purified by reversed-phase HPLC ((column: Phenomenex luna C18 150*25 mm*10 um; mobile phase: [water(FA)-Acetonitrile]; gradient: 12%-42% B over 10 min) to give 4-(10-ethyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl)-5-ethynyl-6-fluoronaphthalen-2-ol (11.1 mg, 0.018 mmol, 37% yield, 99% purity, FA) as brown gum (Ex. 108). ¹H NMR (400 MHz, DMSO-d₆) δ=10.39-9.94 (m, 1H), 7.95 (dd, J=5.6, 9.2 Hz, 1H), 7.45 (t, J=9.2 Hz, 1H), 7.36 (d, J=2.4 Hz, 1H), 7.13 (d, J=2.4 Hz, 1H), 5.39-5.15 (m, 1H), 4.52 (s, 2H), 4.15-4.07 (m, 2H), 4.02 (dd, J=7.6, 10.4 Hz, 1H), 3.98-3.91 (m, 2H), 3.78 (qd, J=6.4, 13.2 Hz, 1H), 3.14-3.04 (m, 2H), 3.01 (s, 1H), 2.88-2.76 (m, 2H), 2.13 (s, 1H), 2.05 (s, 1H), 2.02-1.96 (m, 1H), 1.88-1.73 (m, 3H), 1.25 (t, J=7.2 Hz, 3H); LCMS: (M+1: 576.1).

Ex. 109 was synthesized following General method D, using intermediate I-2-30 in Step 1. The amide functionality was transformed to nitrile under the conditions in Step 2.

Ex. 111 was synthesized following general method D (Step 3 to Step 6), using intermediate I-5-96 and commercially available I-6-111 in Step 3 as shown below.

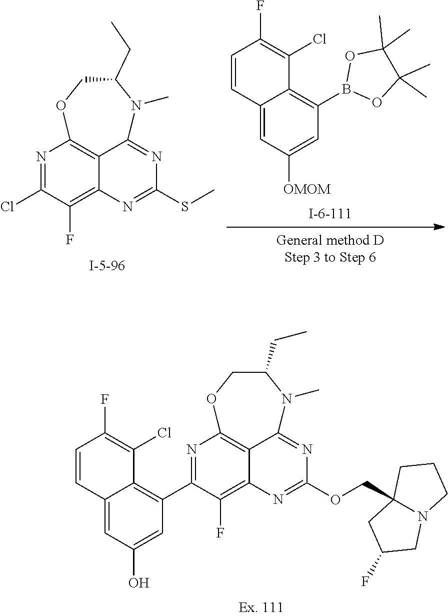

### Preparation of 5-(3-(benzylamino)propyl)-7-chloro-8-fluoro-2-(methylthio)pyrido[4,3-d]pyrimidin-4 (3H)-one (I-4-112)

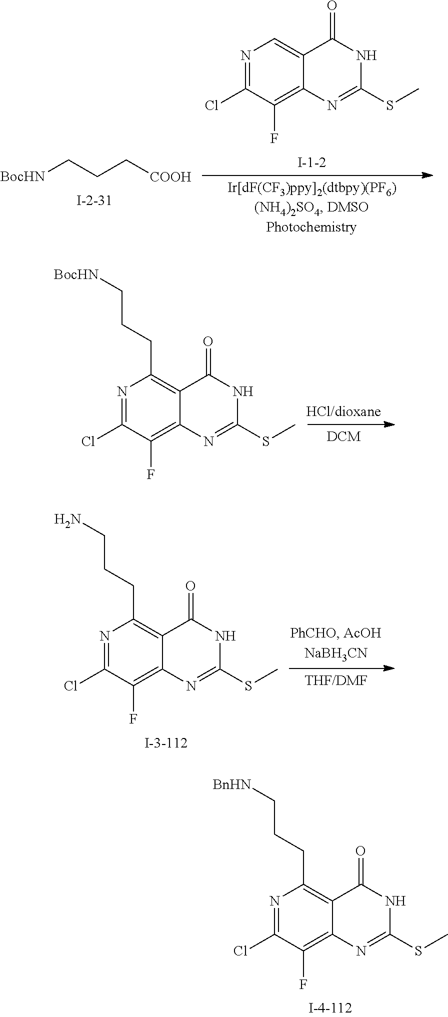

Step 1. To a 15 mL-vial equipped with a stir bar was added I-1-2 (0.200 mmol, 1 eq), I-2-31 (2.00 mmol, 10 eq), Ir[dF(CF₃) ppy]₂(dtbpy)(PF₆) (4.48 mg, 0.02 eq), (NH₄)₂S₂08 (182 mg, 4 eq) in DMSO (2 mL). The vial was sealed and placed under nitrogen. The reaction was stirred and irradiated with a 10W [455 nm] blue LED lamp (3 cm away), with cooling water to keep the reaction temperature at 25° C. for 14 hours. On completion, the mixture was concentrated to give a residue. The residue was purified by reversed-phase HPLC (0.1% FA condition) to give tert-butyl N-[3-(7-chloro-8-fluoro-2-methylsulfanyl-4-oxo-3H-pyrido[4,3-d]pyrimidin-5-yl) propyl]carbamate (500 mg, 1.24 mmol, 15% yield) as a white solid. LCMS: (M+1: 403.0).

Step 2. To a solution of tert-butyl N-[3-(7-chloro-8-fluoro-2-methylsulfanyl-4-oxo-3H-pyrido[4,3-d]pyrimidin-5-yl) propyl]carbamate (500 mg, 1.24 mmol, 1 eq) in DCM (6 mL) was added HCl/dioxane (4 M, 3.1 mL, 10 eq). The mixture was stirred at 25° C. for 0.1 hours. On completion, the mixture was filtered and concentrated to give 5-(3-aminopropyl)-7-chloro-8-fluoro-2-methylsulfanyl-3H-pyrido[4,3-d]pyrimidin-4-one (400 mg, 1.18 mmol, 95% yield, HCl) as a white solid (I-3-112). LCMS: (M+1-17: 285.9).

Step 3. To a solution of 5-(3-aminopropyl)-7-chloro-8-fluoro-2-methylsulfanyl-3H-pyrido[4,3-d]pyrimidin-4-one (400 mg, 1.18 mmol, 1 eq, HCl) and benzaldehyde (112 mg, 1.06 mmol, 0.9 eq) in THF (10 mL) and DMF (2 mL), was added AcOH (3.54 mmol, 0.2 mL, 3 eq) dropwise until pH 5-6 at 25° C. over 0.5 hours. NaBH₃CN (111 mg, 1.77 mmol, 1.5 eq) was then added in portions at 25° C. over 0.5 hours, and the mixture was stirred at 25° C. for 1 hour. On completion, the mixture was quenched with water (10 mL) and extracted with ethyl acetate (5 mL*3), the combined organic phase was dried over anhydrous sodium sulfate, filtered, and concentrated to give a residue. The residue was purified by column chromatography (SiO₂, Petroleum ether/Ethyl acetate=1:0 to 1:1) to give 5-[3-(benzylamino) propyl]-7-chloro-8-fluoro-2-methylsulfanyl-3H-pyrido[4,3-d]pyrimidin-4-one (135 mg, 0.343 mmol, 29% yield) as a white solid (I-4-112). LCMS: (M+1: 393.0).

Ex. 112 was synthesized following General Method F (Steps 2 and 3), using the above intermediate I-4-112 in Step 2, followed by General Method D (Step 4 to Step 6). Alternatively, it will be appreciated by the person skilled in the art that Ex. 112 can be prepared using General Method H followed by General Method D.

### Preparation of (8aS,9S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7-oxa-1,3,6,12a-tetraazabenzo[4,5]cyclohepta[1,2,3-de]naphthalen-9-ol (Ex. 114) and (8aR,9R)-5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7-oxa-1,3,6,12a-tetraazabenzo[4,5]cyclohepta[1,2,3-de]naphthalen-9-ol (Ex. 3)

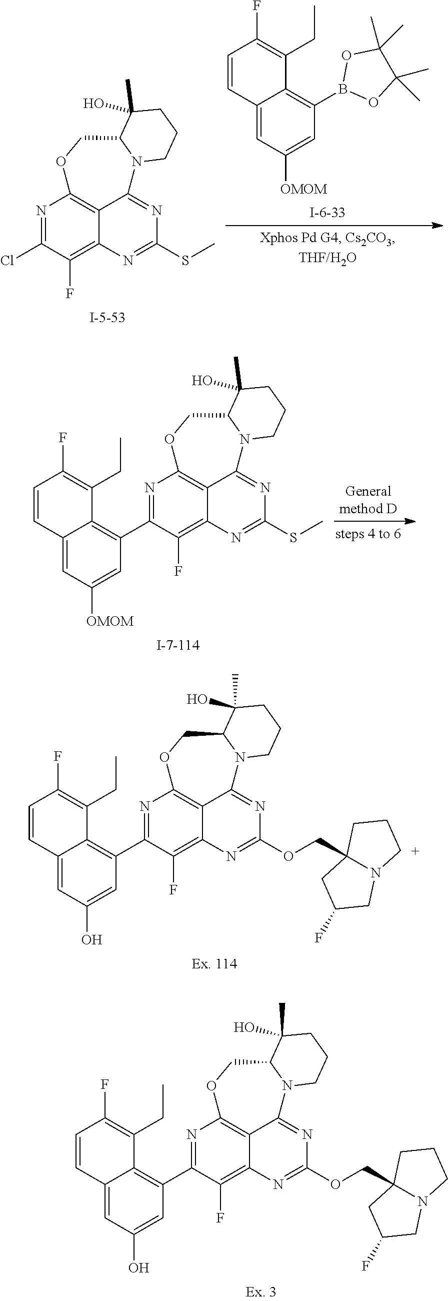

Step 1. A mixture of 2-[8-ethyl-7-fluoro-3-(methoxymethoxy)-1-naphthyl]-4,4,5,5-tetramethyl-1,3,2-dioxaborolane (145 mg, 0.404 mmol, 1.5 eq, I-6-33), (5aR,6R)-2-chloro-1-fluoro-6-methyl-11-(methylthio)-5,5a,6,7,8,9-hexahydro-4-oxa-3,9a,10,12-tetraazabenzo[4,5]cyclohepta[1,2,3-de]naphthalen-6-ol (100 mg, 0269 mmol, 1 eq, I-5-53), XPhos Pd G4 (46.4 mg, 0.054 mmol, 0.2 eq), Cs₂CO₃ (175 mg, 0.539 mmol, 2 eq) in THF (2 mL) and H₂O (0.4 mL) was degassed and purged with N₂ 3 times, and then the mixture was stirred at 80° C. for 2 h under N₂ atmosphere. On completion, the reaction mixture was filtered and concentrated under reduced pressure to give a residue. The residue was purified by prep-TLC (SiO₂, PE:EtOAc=0:1) to give (5aR,6R)-2-(8-ethyl-7-fluoro-3-(methoxymethoxy)naphthalen-1-yl)-1-fluoro-6-methyl-11-(methylthio)-5,5a,6,7,8,9-hexahydro-4-oxa-3,9a,10,12-tetraazabenzo[4,5]cyclohepta[1,2,3-de]naphthalen-6-ol (70.0 mg, 0.116 mmol, 44% yield, 95% purity) as a yellow solid (I-7-114). LCMS: (M+1: 569.0).

Step 2. I-7-114 was converted to Ex. 114 and Ex. 3 following General Method D, Steps 4-6.

### Preparation of tert-butyl N-[3-cyano-4-(4,4,5,5-tetramethyl-1,3,2-dioxaborolan-2-yl) benzothiophen-2-yl]carbamate (I-6-115)

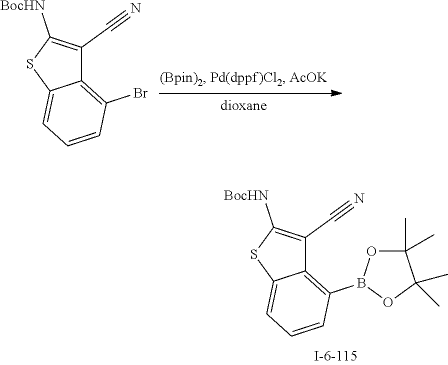 [SMILES: CC(C)(C)OC(=O)Nc1sc2cccc(Br)c2c1C#N ; CC(C)(C)OC(=O)Nc1sc2cccc(B3OC(C)(C)C(C)(C)O3)c2c1C#N]

A mixture of tert-butyl N-(4-bromo-3-cyano-benzothiophen-2-yl) carbamate (120 mg, 0.339 mmol, 1 eq), 4,4,5,5-tetramethyl-2-(4,4,5,5-tetramethyl-1,3,2-dioxaborolan-2-yl)-1,3,2-dioxaborolane (120 mg, 0.475 mmol, 1.4 eq), KOAc (100 mg, 1.02 mmol, 3 eq), Pd(dppf)Cl₂ (24.8 mg, 0.033 mmol, 0.1 eq) in dioxane (3 mL) was degassed and purged with N₂ 3 times, and then the mixture was stirred at 80° C. for 3 hours under N₂ atmosphere. On completion, the mixture was filtered and concentrated to give a residue. The residue was purified by column chromatography (SiO₂, Petroleum ether/Ethyl acetate=1:0 to 5:1) to give tert-butyl N-[3-cyano-4-(4,4,5,5-tetramethyl-1,3,2-dioxaborolan-2-yl) benzothiophen-2-yl]carbamate (80.0 mg, 0.199 mmol, 58% yield) as a white solid (I-6-115). LCMS: (M+1-56: 344.8).

### Preparation of 4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-5,6-difluoronaphthalen-2-ol (Ex. 115)

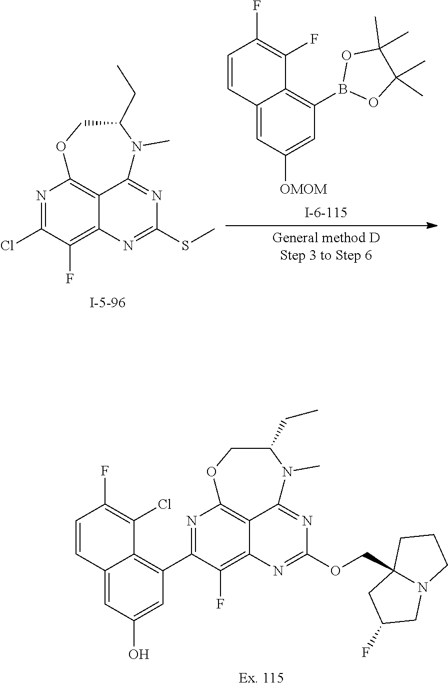

Ex. 115 was synthesized following general method D (Step 3 to Step 6), using intermediate I-5-96 and commercially available I-6-115 in Step 3.

### Preparation of 2-amino-4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-1-benzothiophene-3-carbonitrile (Ex. 116)

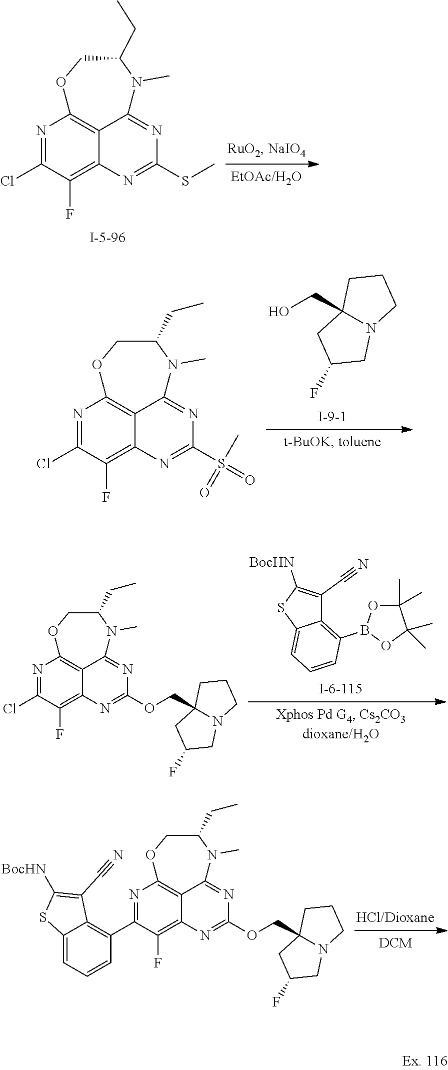 [SMILES: CC[C@H]1COc2nc(Cl)c(F)c3nc(SC)nc(c23)N1C ; CC[C@H]1COc2nc(Cl)c(F)c3nc(S(C)(=O)=O)nc(c23)N1C ; OC[C@@]12CCCN1C[C@H](F)C2 ; CC[C@H]1COc2nc(Cl)c(F)c3nc(OC[C@@]45CCCN4C[C@H](F)C5)nc(c23)N1C ; CC(C)(C)OC(=O)Nc1sc2cccc(B3OC(C)(C)C(C)(C)O3)c2c1C#N ; CC[C@H]1COc2nc(-c3cccc4sc(NC(=O)OC(C)(C)C)c(C#N)c34)c(F)c3nc(OC[C@@]45CCCN4C[C@H](F)C5)nc(c23)N1C]

Step 1. To a solution of (12S)-7-chloro-12-ethyl-6-fluoro-13-methyl-3-methylsulfanyl-10-oxa-2,4,8,13-tetrazatricyclo [7.4.1.05,14]tetradeca-1,3,5,7,9 (14)-pentaene (280 mg, 0.851 mmol, 1 eq, I-5-96) in EtOAc (5 mL) and H₂O (5 mL) was added NaIO₄ (1.82 g, 8.52 mmol, 10 eq) and RuO₂ (226 mg, 1.70 mmol, 2 eq). The mixture was stirred at 25° C. for 1 hour. On completion, the mixture was quenched with the saturated solution of Na₂SO₃ (20 mL) and extracted with ethyl acetate (25 mL*3), the combined organic phase was dried over anhydrous sodium sulfate, filtered, and concentrated to give a residue. The residue was purified by column chromatography (SiO₂, Petroleum ether/THF=1:0 to 1:1) to give (12S)-7-chloro-12-ethyl-6-fluoro-13-methyl-3-methylsulfonyl-10-oxa-2,4,8,13-tetrazatricyclo[7.4.1.05,14]tetradeca-1,3,5,7,9 (14)-pentaene (120 mg, 0.332 mmol, 39% yield) as a white solid. LCMS: (M+1: 361.0).

Step 2. To a solution of [(2R,8S)-2-fluoro-1,2,3,5,6,7-hexahydropyrrolizin-8-yl]methanol (132 mg, 0.831 mmol, 3 eq, I-9-1) in toluene (3 mL) was added t-BuOK (93.3 mg, 0.831 mmol, 3 eq), degassed and purged with N₂ 3 times, and then the mixture was stirred at 25° C. under N₂ atmosphere. (12S)-7-chloro-12-ethyl-6-fluoro-13-methyl-3-methylsulfonyl-10-oxa-2,4,8,13-tetrazatricyclo [7.4.1.05,14]tetradeca-1,3,5,7,9 (14)-pentaene (100 mg, 0.277 mmol, 1 eq) was then added to reaction mixture and stirred at 25° C. for 0.5 hours. On completion, the mixture was quenched with water (10 mL) and extracted with ethyl acetate (5 mL*3), the combined organic phase was dried over anhydrous sodium sulfate, filtered, and concentrated to give a residue. The residue was purified by column chromatography (SiO₂, Petroleum ether/Ethyl acetate=1:0 to 5:1) to give (12S)-7-chloro-12-ethyl-6-fluoro-3-[[(2R,8S)-2-fluoro-1,2,3,5,6,7-hexahydropyrrolizin-8-yl]methoxy]-13-methyl-10-oxa-2,4,8,13-tetrazatricyclo [7.4.1.05,14]tetradeca-1,3,5,7,9 (14)-pentaene (120 mg, 0.272 mmol, 98% yield) as a white solid. LCMS: (M+1: 440.0).

Step 3. A mixture of (12S)-7-chloro-12-ethyl-6-fluoro-3-[[(2R,8S)-2-fluoro-1,2,3,5,6,7-hexahydropyrrolizin-8-yl]methoxy]-13-methyl-10-oxa-2,4,8,13-tetrazatricyclo [7.4.1.05,14]tetradeca-1,3,5,7,9 (14)-pentaene (40.0 mg, 0.091 mmol, 1 eq), tert-butyl N-[3-cyano-4-(4,4,5,5-tetramethyl-1,3,2-dioxaborolan-2-yl)benzothiophen-2-yl]carbamate (40.0 mg, 0.100 mmol, 1.1 eq), Cs₂CO₃ (88.8 mg, 0.272 mmol, 3 eq), XPhos Pd G₄ (7.82 mg, 0.009 mmol, 0.1 eq) in dioxane (2 mL) and H₂O (0.4 mL) was degassed and purged with N₂ 3 times, and then the mixture was stirred at 80° C. for 1 hour under N₂ atmosphere. On completion, the mixture was filtered and concentrated to give a residue. The residue was purified by column chromatography (SiO₂, DCM/MeOH=1:0 to 5:1) to give tert-butyl N-[3-cyano-4-[(12S)-12-ethyl-6-fluoro-3-[[(2R,8S)-2-fluoro-1,2,3,5,6,7-hexahydropyrrolizin-8-yl]methoxy]-13-methyl-10-oxa-2,4,8,13-tetrazatricyclo [7.4.1.05,14]tetradeca-1,3,5,7,9 (14)-pentaen-7-yl]benzothiophen-2-yl]carbamate (12.0 mg, 0.018 mmol, 19% yield) as a white solid. LCMS: (M+1: 678.3).

Step 4. To a solution of tert-butyl N-[3-cyano-4-[(12S)-12-ethyl-6-fluoro-3-[[(2R,8S)-2-fluoro-1,2,3,5,6,7-hexahydropyrrolizin-8-yl]methoxy]-13-methyl-10-oxa-2,4,8,13-tetrazatricyclo [7.4.1.05,14]tetradeca-1,3,5,7,9 (14)-pentaen-7-yl]benzothiophen-2-yl]carbamate (12.0 mg, 0.018 mmol, 1 eq) in DCM (0.5 mL) was added HCl/dioxane (4 M, 10 eq). The mixture was stirred at 25° C. for 0.1 hour. On completion, the mixture was concentrated to give a residue. The residue was purified by prep-HPLC (column: Phenomenex luna C18 150*25 mm*10 um; mobile phase: [water(FA)-ACN]; gradient: 10%-40% B over 11 min) to give 2-amino-4-[(12S)-12-ethyl-6-fluoro-3-[[(2R,8S)-2-fluoro-1,2,3,5,6,7-hexahydropyrrolizin-8-yl]methoxy]-13-methyl-10-oxa-2,4,8,13-tetrazatricyclo [7.4.1.05,14]tetradeca-1,3,5,7,9 (14)-pentaen-7-yl]benzothiophene-3-carbonitrile (1.82 mg, 0.0029 mmol, 16.25% yield, 98.62% purity, FA) as a pink solid (Ex. 116). ¹H NMR (400 MHz, DMSO-d₆) δ=7.84-7.70 (m, 3H), 7.32 (d, J=7.6 Hz, 1H), 7.25-7.16 (m, 1H), 5.38-5.17 (m, 1H), 4.71-4.60 (m, 1H), 4.37 (d, J=13.6 Hz, 1H), 4.18-3.98 (m, 2H), 3.95-3.83 (m, 1H), 3.36 (s, 3H), 3.09 (d, J=7.6 Hz, 2H), 3.02 (s, 1H), 2.87-2.77 (m, 1H), 2.13 (d, J=6.4 Hz, 1H), 2.07-1.99 (m, 2H), 1.86-1.64 (m, 5H), 1.03-0.94 (m, 3H); LCMS: (M+1: 578.0).

### Preparation of 5-ethyl-6-fluoro-4-(4-fluoro-2-{[(2R,7aS)-2-fluorotetrahydro-1H-pyrrolizin-7a(5H)-yl]methoxy}-7,8,9,10-tetrahydro-1,3,6,10-tetraazacyclohepta[1,2,3-de]naphthalen-5-yl)naphthalen-2-ol (Ex. 117)

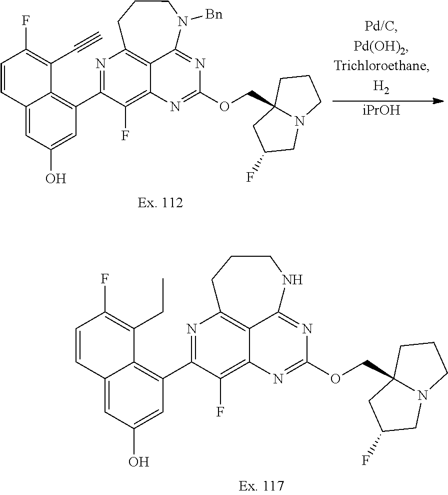 [SMILES: "iPrOH" ; C#Cc1c(F)ccc2cc(O)cc(-c3nc4c5c(nc(OC[C@@]67CCCN6C[C@H](F)C7)nc5c3F)N(Cc3ccccc3)CCC4)c12 ; CCc1c(F)ccc2cc(O)cc(-c3nc4c5c(nc(OC[C@@]67CCCN6C[C@H](F)C7)nc5c3F)NCCC4)c12]

To a solution of 4-[13-benzyl-6-fluoro-3-[[(2R,8S)-2-fluoro-1,2,3,5,6,7-hexahydropyrrolizin-8-yl]methoxy]-2,4,8,13-tetrazatricyclo [7.4.1.05,14]tetradeca-1,3,5,7,9 (14)-pentaen-7-yl]-5-ethynyl-6-fluoro-naphthalen-2-ol (18.0 mg, 0.028 mmol, 1 eq, Ex. 112) in i-PrOH (1 ml) was added Pd/C (50.0 mg, 10% purity), Pd(OH)₂ (50.0 mg, 20% purity) and 1,1,2-Trichloroethane (4.16 mg, 0.031 mmol, 1.1 eq) under N₂ atmosphere. The suspension was degassed and purged with H₂ 3 times. The mixture was stirred under H₂ (15 psi) at 25° C. for 48 hours. On completion, the mixture was concentrated to give a residue. The residue was purified by prep-HPLC (column: Phenomenex luna C18 150*25 mm*10 um; mobile phase: [water(FA)-ACN]; gradient: 13%-43% B over 10 min) to give 5-ethyl-6-fluoro-4-[6-fluoro-3-[[(2R,8S)-2-fluoro-1,2,3,5,6,7-hexahydropyrrolizin-8-yl]methoxy]-2,4,8,13-tetrazatricyclo[7.4.1.05,14]tetradeca-1,3,5,7,9 (14)-pentaen-7-yl]naphthalen-2-ol (1.08 mg, 0.0018 mmol, 6.38% yield, 99.64% purity, FA) as an off-white solid (Ex. 117). ¹H NMR (400 MHz, DMSO-d₆) δ=9.07-8.71 (m, 1H), 8.39 (s, 1H), 7.80-7.66 (m, 1H), 7.39-7.28 (m, 2H), 7.00 (d, J=2.4 Hz, 1H), 5.40-5.14 (m, 1H), 4.13-3.98 (m, 2H), 3.25-3.20 (m, 3H), 3.11-3.06 (m, 2H), 3.01 (s, 1H), 2.87-2.77 (m, 1H), 2.30-2.18 (m, 2H), 2.13-2.08 (m, 2H), 2.04-1.96 (m, 2H), 1.87-1.73 (m, 3H), 1.28-1.19 (m, 2H), 0.81-0.73 (m, 3H); LCMS: (M+1: 550.2).

### Preparation of (8aS,11R)-5-chloro-4-fluoro-2-(methylthio)-8,8a,9,10,11,12-hexahydro-7-oxa-1,3,6,12a-tetraazabenzo[4,5]cyclohepta[1,2,3-de]naphthalene-11-carbonitrile (I-5-118)

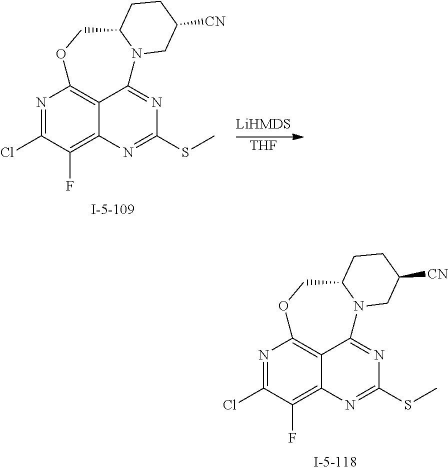 [SMILES: CSc1nc2c3c(nc(Cl)c(F)c3n1)OC[C@@H]1CC[C@H](C#N)CN21 ; CSc1nc2c3c(nc(Cl)c(F)c3n1)OC[C@@H]1CC[C@@H](C#N)CN21 ; "THF"]

To a solution of (4S,7S)-12-chloro-13-fluoro-16-methylsulfanyl-9-oxa-2,11,15,17-tetrazatetracyclo[8.7.1.02,7.014,18]octadeca-1 (17),10 (18),11,13,15-pentaene-4-carbonitrile (105 mg, 0.287 mmol, 1 eq, from the synthesis of Ex. 109, mixture of cis isomers) in THE (2 mL) was added LiHMDS (1.0 M, 0.287 mL, 1 eq). The mixture was stirred at 25° C. for 1 h. On completion, the mixture was quenched with water (20 mL) and extracted with ethyl acetate (25 mL×3), the combined organic phase was dried over anhydrous sodium sulfate, filtered, and concentrated to give a residue. The residue was purified by column chromatography (SiO₂, Petroleum ether/Ethyl acetate=1:0 to 0:1) to give (4R,7S)-12-chloro-13-fluoro-16-methylsulfanyl-9-oxa-2,11,15,17-tetrazatetracyclo[8.7.1.02,7.014,18]octadeca-1 (17),10 (18),11,13,15-pentaene-4-carbonitrile (100 mg, 0.273 mmol, 95% yield) as white solid (I-5-118) as a mixture trans isomers. ¹H NMR (400 MHz, DMSO-d₆) δ=4.60-4.43 (m, 2H), 3.76 (br s, 1H), 3.66-3.57 (m, 1H), 2.55-2.52 (m, 1H), 2.43 (br s, 2H), 1.84-1.74 (m, 2H), 1.29-1.23 (m, 2H); LCMS: (M+1=365.9).

Ex. 118 was synthesized following General method D (Steps 3-6), using above intermediate I-5-118 in Step 3.

Ex. 119 was prepared following General Method D, using intermediate I-2-4 in Step 1, and using intermediate I-6-33 in Step 3.

Ex. 121 was synthesized following General Method H, followed by General Method D (Step 3 to Step 6).

### Preparation of 5-ethynyl-6-fluoro-4-(4-fluoro-2-{[(2R,7aS)-2-fluorotetrahydro-1H-pyrrolizin-7a(5H)-yl]methoxy}-7,8,9,10-tetrahydro-1,3,6,10-tetraazacyclohepta[1,2,3-de]naphthalen-5-yl)naphthalen-2-ol (Ex. 122)

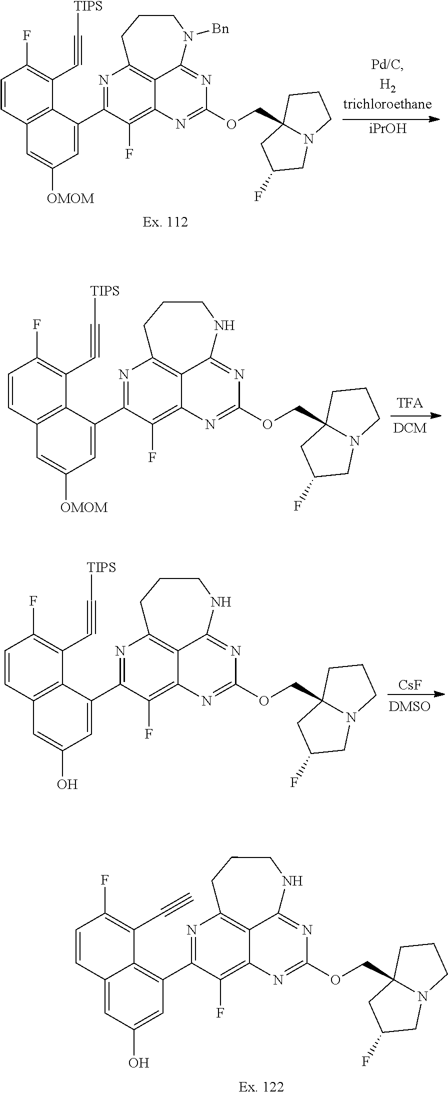

Step 1. To a solution of 2-[8-[13-benzyl-6-fluoro-3-[[(2R,8S)-2-fluoro-1,2,3,5,6,7-hexahydropyrrolizin-8-yl]methoxy]-2,4,8,13-tetrazatricyclo [7.4.1.05,14]tetradeca-1,3,5,7,9 (14)-pentaen-7-yl]-2-fluoro-6-(methoxymethoxy)-1-naphthyl]ethynyl-triisopropyl-silane (40.0 mg, 0.048 mmol, 1 eq, Ex. 112) in i-PrOH (1 mL) was added Pd/C (50.0 mg, 10% purity) and 1,1,2-trichloroethane (7.02 mg, 0.053 mmol, 1.1 eq) under N₂ atmosphere. The suspension was degassed and purged with H₂ 3 times. The mixture was stirred under H2 (15 psi) at 25° C. for 14 hours. On completion, the mixture was concentrated to give 2-[2-fluoro-8-[6-fluoro-3-[[(2R,8S)-2-fluoro-1,2,3,5,6,7-hexahydropyrrolizin-8-yl]methoxy]-2,4,8,13-tetrazatricyclo [7.4.1.05,14]tetradeca-1,3,5,7,9 (14)-pentaen-7-yl]-6-(methoxymethoxy)-1-naphthyl]ethynyl-triisopropyl-silane (35.0 mg, 0.047 mmol, 98% yield) as a yellow solid. LCMS: (M+1: 746.4).

Step 2. To a solution of 2-[2-fluoro-8-[6-fluoro-3-[[(2R,8S)-2-fluoro-1,2,3,5,6,7-hexahydropyrrolizin-8-yl]methoxy]-2,4,8,13-tetrazatricyclo [7.4.1.05,14]tetradeca-1,3,5,7,9 (14)-pentaen-7-yl]-6-(methoxymethoxy)-1-naphthyl]ethynyl-triisopropyl-silane (31.2 mg, 0.042 mmol, 1 eq) in DCM (0.5 mL) was added TFA (47.7 mg, 0.418 mmol, 10 eq). The mixture was stirred at 25° C. for 0.1 hours. On completion, the mixture was concentrated to give a residue. The residue was purified by prep-HPLC (column: Phenomenex luna C18 150*25 mm*10 um; mobile phase: [water(FA)-ACN]; gradient: 34%-64% B over 11 min) to give 6-fluoro-4-[6-fluoro-3-[[(2R,8S)-2-fluoro-1,2,3,5,6,7-hexahydropyrrolizin-8-yl]methoxy]-2,4,8,13-tetrazatricyclo [7.4.1.05,14]tetradeca-1,3,5,7,9 (14)-pentaen-7-yl]-5-(2-triisopropylsilylethynyl) naphthalen-2-ol (8.00 mg, 0.011 mmol, 27% yield) as a white solid. LCMS: (M+1: 702.3).

Step 3. To a solution of 6-fluoro-4-[6-fluoro-3-[[(2R,8S)-2-fluoro-1,2,3,5,6,7-hexahydropyrrolizin-8-yl]methoxy]-2,4,8,13-tetrazatricyclo [7.4.1.05,14]tetradeca-1,3,5,7,9 (14)-pentaen-7-yl]-5-(2-triisopropylsilylethynyl) naphthalen-2-ol (8.00 mg, 0.011 mmol, 1 eq) in DMSO (0.5 mL) was added CsF (10.3 mg, 0.068 mmol, 6 eq). The mixture was stirred at 25° C. for 0.5 hours. On completion, the mixture was concentrated to give a residue. The residue was purified by prep-HPLC (column: Phenomenex luna C18 150*25 mm*10 um; mobile phase: [water(FA)-ACN]; gradient: 18%-38% B over 10 min) to give 5-ethynyl-6-fluoro-4-[6-fluoro-3-[[(2R,8S)-2-fluoro-1,2,3,5,6,7-hexahydropyrrolizin-8-yl]methoxy]-2,4,8,13-tetrazatricyclo [7.4.1.05,14]tetradeca-1,3,5,7,9 (14)-pentaen-7-yl]naphthalen-2-ol (0.750 mg, 0.0011 mmol, 9.93% yield, 99.53% purity, TFA) as a yellow solid (Ex. 122). ¹H NMR (400 MHz, DMSO-d₆) δ=10.17 (s, 1H), 9.17-8.88 (m, 1H), 8.05-7.89 (m, 1H), 7.49-7.43 (m, 1H), 7.38 (d, J=2.4 Hz, 1H), 7.23-7.16 (m, 2H), 6.68-6.61 (m, 1H), 5.69-5.44 (m, 1H), 5.34-5.30 (m, 2H), 4.64-4.45 (m, 2H), 3.83-3.64 (m, 3H), 3.21 (s, 2H), 2.20-2.09 (m, 5H), 1.48-1.43 (m, 3H); LCMS: (M+1: 546.2).

### Preparation of 5-ethyl-6-fluoro-4-[(9S)-4-fluoro-2-{[(2R,7aS)-2-fluorotetrahydro-1H-pyrrolizin-7a(5H)-yl]methoxy}-9,10-dimethyl-9,10-dihydro-8H-7-oxa-1,3,6,10-tetraazacyclohepta[1,2,3-de]naphthalen-5-yl]naphthalen-2-ol (Ex. 123)

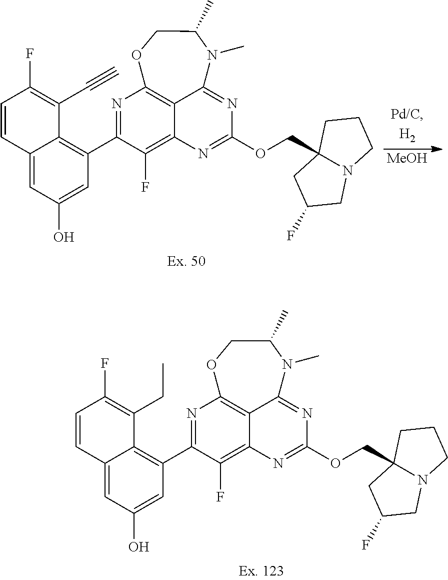 [SMILES: "MeOH" ; C#Cc1c(F)ccc2cc(O)cc(-c3nc4c5c(nc(OC[C@@]67CCCN6C[C@H](F)C7)nc5c3F)N(C)[C@@H](C)CO4)c12 ; CCc1c(F)ccc2cc(O)cc(-c3nc4c5c(nc(OC[C@@]67CCCN6C[C@H](F)C7)nc5c3F)N(C)[C@@H](C)CO4)c12]

To a solution of 5-ethynyl-6-fluoro-4-[(12S)-6-fluoro-3-[[(2R,8S)-2-fluoro-1,2,3,5,6,7-hexahydropyrrolizin-8-yl]methoxy]-12,13-dimethyl-10-oxa-2,4,8,13-tetrazatricyclo [7.4.1.05,14]tetradeca-1,3,5,7,9 (14)-pentaen-7-yl]naphthalen-2-ol (50.0 mg, 0.087 mmol, 1 eq) in MeOH (1 mL) was added Pd/C (125 mg, 0.117 mmol, 10% purity, 1.35 eq) under N₂ atmosphere. The suspension was degassed and purged with H₂ 3 times. The mixture was stirred under H₂ (15 psi) at 25° C. for 12 h. On completion, the mixture was filtered to remove Pd/C. The filtrate was purified by prep-HPLC (column: Welch Ultimate C_{18 150*25} mm*5 um; mobile phase: [water(FA)-ACN]; gradient: 15%-45% B over 10 min) to give 5-ethyl-6-fluoro-4-[(12S)-6-fluoro-3-[[(2R,8S)-2-fluoro-1,2,3,5,6,7-hexahydropyrrolizin-8-yl]methoxy]-12,13-dimethyl-10-oxa-2,4,8,13-tetrazatricyclo[7.4.1.05,14]tetradeca-1,3,5,7,9 (14)-pentaen-7-yl]naphthalen-2-ol (4.67 mg, 0.0074 mmol, 8.56% yield, 99.663% purity, FA) as a white solid (Ex. 123). ¹H NMR (400 MHz, DMSO-d₆) δ=10.10-9.76 (m, 1H), 7.78-7.69 (m, 1H), 7.40-7.25 (m, 2H), 6.96 (d, J=2.4 Hz, 1H), 5.41-5.15 (m, 1H), 4.58-4.40 (m, 2H), 4.16-4.02 (m, 2H), 3.36 (br s, 3H), 3.14-2.97 (m, 3H), 2.89-2.77 (m, 1H), 2.30-2.10 (m, 2H), 2.06 (br s, 2H), 1.88-1.70 (m, 3H), 1.28 (d, J=6.8 Hz, 3H), 0.89-0.72 (m, 3H); LCMS: (M+1=580.2).

### Preparation of 5-ethyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-9,10-dihydro-8H-7-oxa-1,3,6,10-tetraazacyclohepta[1,2,3-de]naphthalen-5-yl]naphthalen-2-ol (Ex. 124)

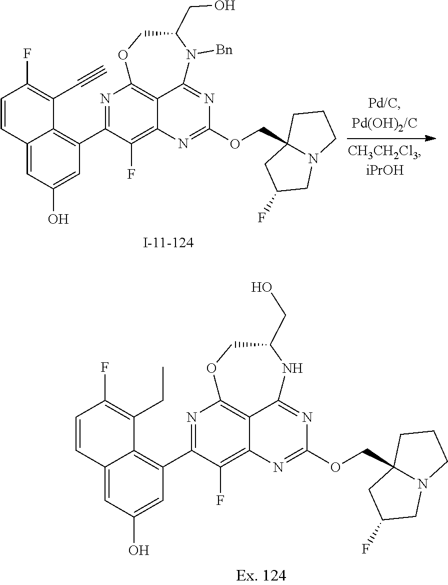 [SMILES: C#Cc1c(F)ccc2cc(O)cc(-c3nc4c5c(nc(OC[C@@]67CCCN6C[C@H](F)C7)nc5c3F)N(Cc3ccccc3)[C@@H](CO)CO4)c12 ; CCc1c(F)ccc2cc(O)cc(-c3nc4c5c(nc(OC[C@@]67CCCN6C[C@H](F)C7)nc5c3F)N[C@@H](CO)CO4)c12]

I-11-124 was synthesized following General Method D, using I-2-33 in Step 1. To a solution of I-11-124 (200 mg, 0.300 mmol, 1 eq) in i-PrOH (4 mL) was added Pd/C (80.0 mg, 0.075 mmol, 10% purity, 0.25 eq) and Pd(OH)₂ (79.9 mg, 0.114 mmol, 20% purity, 0.38 eq). The mixture was stirred at 25° C. for 18 h. On completion, the mixture was filtered and concentrated to give a residue. The residue was purified by prep-HPLC (neutral condition; column: Waters Xbridge 150*25 mm*5 um; mobile phase: [water (NH₄HCO₃)-ACN]; gradient: 24%-54% B over 10 min) to give 5-ethyl-6-fluoro-4-[(12S)-6-fluoro-3-[[(2R,8S)-2-fluoro-1,2,3,5,6,7-hexahydropyrrolizin-8-yl]methoxy]-12-(hydroxymethyl)-10-oxa-2,4,8,13-tetrazatricyclo[7.4.1.0^{5,14}]tetradeca-1,3,5,7, 9(14)-pentaen-7-yl]naphthalen-2-ol (8.97 mg, 0.0154 mmol, 5.14% yield) as an off white solid (Ex. 124). ¹H NMR (400 MHz, MeOD-d₄) δ=7.70-7.62 (m, 1H), 7.31-7.20 (m, 2H), 7.07 (br d, J=11.2 Hz, 1H), 5.46-5.29 (m, 1H), 4.75-4.66 (m, 1H), 4.66-4.53 (m, 2H), 4.43-4.31 (m, 2H), 3.98-3.89 (m, 1H), 3.87-3.72 (m, 2H), 3.56-3.37 (m, 3H), 3.20-3.10 (m, 1H), 2.58-2.45 (m, 1H), 2.40-2.24 (m, 3H), 2.22-2.14 (m, 1H), 2.14-1.90 (m, 3H), 0.91-0.83 (m, 3H); LCMS: (M+1: 582.3).

Ex. 125 was synthesized following General Method D, using I-2-34 in Step 1. Ex. 125 was obtained as the major product in Step 6.

### Preparation of 4-[(9S)-10-ethyl-4-fluoro-2-{[(2R,7aS)-2-fluorotetrahydro-1H-pyrrolizin-7a(5H)-yl]methoxy}-9-methyl-9,10-dihydro-8H-7-oxa-1,3,6,10-tetraazacyclohepta[1,2,3-de]naphthalen-5-yl]-5-ethynyl-6-fluoronaphthalen-2-ol (Ex. 127)

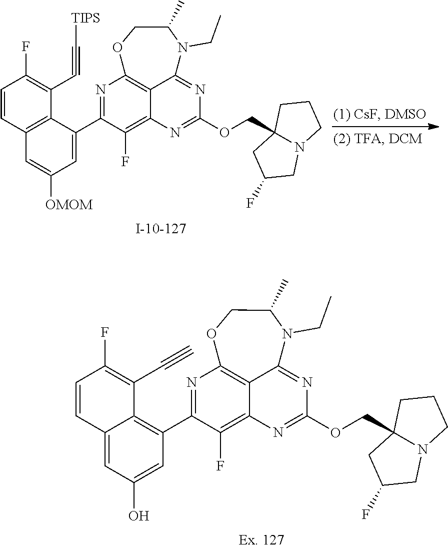

Intermediate I-10-127 was prepared following General Method F using I-2-36, followed by General Method D (Steps 4 and 5). To a solution of I-10-127 (40.0 mg, 0.051 mmol, 1 eq) in DMSO (3 mL) was added CsF (38.4 mg, 0.253 mmol, 5 eq). The mixture was stirred at 0° C. for 1 hour. On completion, the reaction mixture was quenched by addition of H₂O (10 mL) and extracted with EA (5 mL*3). The combined organic layers were washed with saturated solution of NaCl (10 mL), filtered and concentrated under reduced pressure to give the compound (12S)-13-ethyl-7-[8-ethynyl-7-fluoro-3-(methoxymethoxy)-1-naphthyl]-6-fluoro-3-[[(2R,8S)-2-fluoro-1,2,3,5,6,7-hexahydropyrrolizin-8-yl]methoxy]-12-methyl-10-oxa-2,4,8,13-tetrazatricyclo[7.4.1.0^{5,14}]tetradeca-1,3,5,7,9 (14)-pentaene (30.0 mg, 0.047 mmol, 94% yield) as a white solid. LCMS: (M+1:634.3).

To a solution of above (12S)-13-ethyl-7-[8-ethynyl-7-fluoro-3-(methoxymethoxy)-1-naphthyl]-6-fluoro-3-[[(2R,8S)-2-fluoro-1,2,3,5,6,7-hexahydropyrrolizin-8-yl]methoxy]-12-methyl-10-oxa-2,4,8,13-tetrazatricyclo[7.4.1.0^{5,14}]tetradeca-1,3,5,7,9 (14)-pentaene (30.0 mg, 0.047 mmol, 1 eq) in DCM (2 mL) was added 2,2,2-trifluoroacetic acid (26.9 mg, 0.236 mmol, 5 eq). The mixture was stirred at 0° C. for 1 hour. On completion, the reaction mixture was filtered, and the filtrate was concentrated under reduced pressure to remove solvent. The crude product was purified by reversed-phase column: Waters Xbridge BEH C18 150*25 mm*5 um; mobile phase: [water(NH₄HCO₃)-ACN]; gradient: 33%-63% B over 10 min to give compound 4-[(12S)-13-ethyl-6-fluoro-3-[[(2R,8S)-2-fluoro-1,2,3,5,6,7-hexahydropyrrolizin-8-yl]methoxy]-12-methyl-10-oxa-2,4,8,13-tetrazatricyclo[7.4.1.0^{5,14}]tetradeca-1,3,5,7,9 (14)-pentaen-7-yl]-5-ethynyl-6-fluoro-naphthalen-2-ol (11.53 mg, 0.018 mmol, 38.41% yield, 93% purity) as a white solid (Ex. 127). ¹H NMR (400 MHz, DMSO-d6) δ=10.18-10.10 (m, 1H), 7.96 (dd, J=5.6, 9.2 Hz, 1H), 7.50-7.42 (m, 1H), 7.40-7.34 (m, 1H), 7.22-7.08 (m, 1H), 5.39-5.19 (m, 1H), 4.61-4.50 (m, 1H), 4.34 (d, J=12.4 Hz, 1H), 4.20-4.08 (m, 3H), 4.08-3.98 (m, 2H), 3.65-3.47 (m, 1H), 3.14-2.99 (m, 3H), 2.88-2.79 (m, 1H), 2.17-1.97 (m, 3H), 1.91-1.73 (m, 3H), 1.34-1.25 (m, 6H). LCMS: (M+1:590.2).

### Preparation of 4-[(9S)-10-(2,2-difluoroethyl)-4-fluoro-2-{[(2R,7aS)-2-fluorotetrahydro-1H-pyrrolizin-7a(5H)-yl]methoxy}-9-methyl-9,10-dihydro-8H-7-oxa-1,3,6,10-tetraazacyclohepta[1,2,3-de]naphthalen-5-yl]-5-ethynyl-6-fluoronaphthalen-2-ol (Ex. 128)

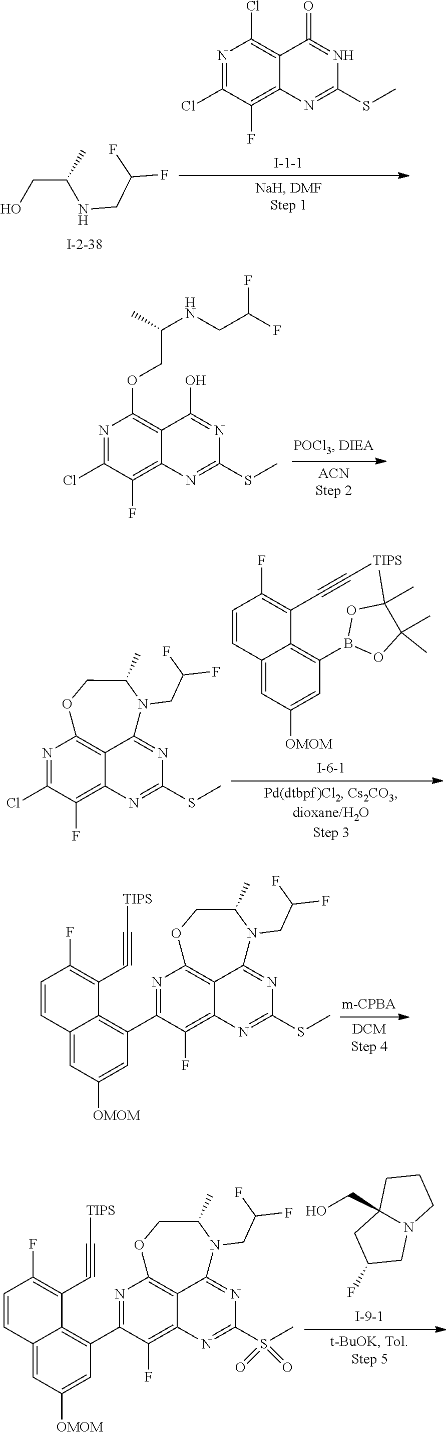

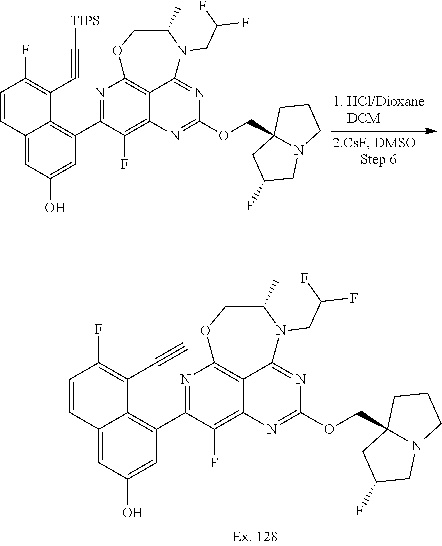

Step 1. To a solution of (2S)-2-(2,2-difluoroethylamino)propan-1-ol (1.27 g, 9.13 mmol, 1 eq, I-2-38) in THF (15 mL) was added NaH (1.10 g, 27.4 mmol, 60% purity, 3 eq). The mixture was stirred at 0° C. for 0.5 hr. Then 5,7-dichloro-8-fluoro-2-methylsulfanyl-3H-pyrido[4,3-d]pyrimidin-4-one (1.28 g, 4.56 mmol, 0.5 eq, I-1-1) was added. The mixture was stirred at 25° C. for 1.5 h. On completion, the reaction mixture was quenched by addition H₂O (5 mL) at 0° C. The crude product was purified by reversed-phase HPLC (neutral condition) to give 7-chloro-5-[(2S)-2-(2,2-difluoroethylamino)propoxy]-8-fluoro-2-methylsulfanyl-pyrido[4,3-d]pyrimidin-4-ol (700 mg, 1.55 mmol, 17% yield, 85% purity) as a white solid. LCMS: (M+23=405.0).

Step 2. To a solution of 7-chloro-5-[(2S)-2-(2,2-difluoroethylamino)propoxy]-8-fluoro-2-methylsulfanyl-pyrido[4,3-d]pyrimidin-4-ol (350 mg, 0.914 mmol, 1 eq) in ACN (3.5 mL) was added POCl₃ (420 mg, 2.74 mmol, 3 eq) and DIEA (1.06 g, 8.23 mmol, 9 eq). The mixture was stirred at 70° C. for 8 h. On completion, the mixture was concentrated in vacuum to give crude. The residue was purified by column chromatography (SiO₂, Petroleum ether/Ethyl acetate=4:1) to give (12S)-7-chloro-13-(2,2-difluoroethyl)-6-fluoro-12-methyl-3-methylsulfanyl-10-oxa-2,4,8,13-tetrazatricyclo[7.4.1.05,14]tetradeca-1,3,5,7,9 (14)-pentaene (250 mg, 0.609 mmol, 66% yield, 89% purity) as a white solid. LCMS: (M+1=365.0).

Step 3. A mixture of (12S)-7-chloro-13-(2,2-difluoroethyl)-6-fluoro-12-methyl-3-methylsulfanyl-10-oxa-2,4,8,13-tetrazatricyclo[7.4.1.05,14]tetradeca-1,3,5,7,9 (14)-pentaene (230 mg, 0.630 mmol, 1 eq), 2-[2-fluoro-6-(methoxymethoxy)-8-(4,4,5,5-tetramethyl-1,3,2-dioxaborolan-2-yl)-1-naphthyl]ethynyl-triisopropyl-silane (484 mg, 0.945 mmol, 1.5 eq, I-6-1), Cs₂CO₃ (616 mg, 1.89 mmol, 3 eq), di-tert-butyl(cyclopentyl)phosphane; dichloropalladium; iron (41.1 mg, 0.063 mmol, 0.1 eq) in dioxane (6 mL) and H₂O (1.2 mL) was degassed and purged with N₂ 3 times, and then the mixture was stirred at 80° C. for 2 h under N₂ atmosphere. On completion, the mixture was filtered and concentrated to give a residue. The residue was purified by column chromatography (SiO₂, Petroleum ether/Ethyl acetate=3:1) to give 2-[8-[(12S)-13-(2,2-difluoroethyl)-6-fluoro-12-methyl-3-methylsulfanyl-10-oxa-2,4,8,13-tetrazatricyclo[7.4.1.05,14]tetradeca-1,3,5,7,9 (14)-pentaen-7-yl]-2-fluoro-6-(methoxymethoxy)-1-naphthyl]ethynyl-triisopropyl-silane (400 mg, 0.553 mmol, 87% yield, 99% purity) as a yellow solid. LCMS: (M+1=715.3).

Step 4. To a solution of 2-[8-[(12S)-13-(2,2-difluoroethyl)-6-fluoro-12-methyl-3-methylsulfanyl-10-oxa-2,4,8,13-tetrazatricyclo [7.4.1.05,14]tetradeca-1,3,5,7,9 (14)-pentaen-7-yl]-2-fluoro-6-(methoxymethoxy)-1-naphthyl]ethynyl-triisopropyl-silane (360 mg, 0.503 mmol, 1 eq) in DCM (3.6 mL) was added m-CPBA (255 mg, 1.26 mmol, 85% purity, 2.5 eq) at 0° C. Then the mixture was stirred at 25° C. for 2 hr. On completion, the reaction mixture was quenched by addition sat. Na₂SO₃ (5 mL) at 0° C., and then diluted with H₂O (5 mL) and extracted with DCM (5 mL×3). The combined organic layers were filtered and concentrated under reduced pressure to give 2-[8-[(12S)-13-(2,2-difluoroethyl)-6-fluoro-12-methyl-3-methylsulfonyl-10-oxa-2,4,8,13-tetrazatricyclo[7.4.1.05,14]tetradeca-1,3,5,7,9 (14)-pentaen-7-yl]-2-fluoro-6-(methoxymethoxy)-1-naphthyl]ethynyl-triisopropyl-silane (350 mg, 0.398 mmol, 79% yield, 85% purity) as a brown solid. LCMS: (M+1=747.3).

Step 5. A mixture of [(2R,8S)-2-fluoro-1,2,3,5,6,7-hexahydropyrrolizin-8-yl]methanol (204 mg, 1.29 mmol, 3 eq, I-9-1), t-BuOK (144 mg, 1.29 mmol, 3 eq) in Toluene (3.5 mL) was degassed and purged with N₂ 3 times, and then the mixture was stirred at 0° C. for 0.5 h under N₂ atmosphere. Then 2-[8-[(12S)-13-(2,2-difluoroethyl)-6-fluoro-12-methyl-3-methylsulfonyl-10-oxa-2,4,8,13-tetrazatricyclo [7.4.1.05,14]tetradeca-1,3,5,7,9 (14)-pentaen-7-yl]-2-fluoro-6-(methoxymethoxy)-1-naphthyl]ethynyl-triisopropyl-silane (320 mg, 0.428 mmol, 1 eq) was added. The mixture was stirred at 25° C. for 1.5 h under N₂ atmosphere. On completion, the mixture was diluted with water (10 mL) and extracted with ethyl acetate (10 mL×3), the combined organic phase was dried over anhydrous sodium sulfate, filtered, and concentrated to give a residue. The residue was purified by column chromatography (SiO₂, Petroleum ether/THF=1:1) to give 2-[8-[(12S)-13-(2,2-difluoroethyl)-6-fluoro-3-[[(2R,8S)-2-fluoro-1,2,3,5,6,7-hexahydropyrrolizin-8-yl]methoxy]-12-methyl-10-oxa-2,4,8,13-tetrazatricyclo [7.4.1.05,14]tetradeca-1,3,5,7,9 (14)-pentaen-7-yl]-2-fluoro-6-(methoxymethoxy)-1-naphthyl]ethynyl-triisopropyl-silane (200 mg, 0.256 mmol, 60% yield, 67% purity) as a white solid. LCMS: (M+1=826.4).

Step 6-1. To a solution of 2-[8-[(12S)-13-(2,2-difluoroethyl)-6-fluoro-3-[[(2R,8S)-2-fluoro-1,2,3,5,6,7-hexahydropyrrolizin-8-yl]methoxy]-12-methyl-10-oxa-2,4,8,13-tetrazatricyclo[7.4.1.05,14]tetradeca-1,3,5,7,9 (14)-pentaen-7-yl]-2-fluoro-6-(methoxymethoxy)-1-naphthyl]ethynyl-triisopropyl-silane (170 mg, 0.205 mmol, 1 eq) in DCM (1.5 mL) was added HCl/dioxane (4 M, 0.051 mL, 1 eq). The mixture was stirred at 25° C. for 2 hr. On completion, the mixture was filtered and concentrated to give 4-[(12S)-13-(2,2-difluoroethyl)-6-fluoro-3-[[(2R,8S)-2-fluoro-1,2,3,5,6,7-hexahydropyrrolizin-8-yl]methoxy]-12-methyl-10-oxa-2,4,8,13-tetrazatricyclo[7.4.1.05,14]tetradeca-1,3,5,7,9 (14)-pentaen-7-yl]-6-fluoro-5-(2-triisopropylsilylethynyl)naphthalen-2-ol (160 mg, 0.204 mmol, 99% yield, crude) as yellow solid. LCMS: (M+1=782.4).

Step 6-2. To a solution of the above 4-[(12S)-13-(2,2-difluoroethyl)-6-fluoro-3-[[(2R,8S)-2-fluoro-1,2,3,5,6,7-hexahydropyrrolizin-8-yl]methoxy]-12-methyl-10-oxa-2,4,8,13-tetrazatricyclo[7.4.1.05,14]tetradeca-1,3,5,7,9 (14)-pentaen-7-yl]-6-fluoro-5-(2-triisopropylsilylethynyl)naphthalen-2-ol (160 mg, 0.204 mmol, 1 eq) in DMSO (1.5 mL) was added Cesium fluoride (186 mg, 1.23 mmol, 6 eq). The mixture was stirred at 25° C. for 2 hr. On completion, the mixture was diluted with water (30 mL) and extracted with ethyl acetate (15 mL×3), the combined organic phase was dried over anhydrous sodium sulfate, filtered, and concentrated to give a residue. The residue purified by prep-HPLC (column: Waters Xbridge BEH C18 150*25 mm*5 um; mobile phase: [water(NH₄HCO₃)-ACN]; gradient: 35%-65% B over 10 min) to furnish 4-[(12S)-13-(2,2-difluoroethyl)-6-fluoro-3-[[(2R,8S)-2-fluoro-1,2,3,5,6,7-hexahydropyrrolizin-8-yl]methoxy]-12-methyl-10-oxa-2,4,8,13-tetrazatricyclo[7.4.1.05,14]tetradeca-1,3,5,7,9 (14)-pentaen-7-yl]-5-ethynyl-6-fluoro-naphthalen-2-ol (18.51 mg, 0.029 mmol, 14.03% yield, 97% purity) as an off-white solid (Ex. 128). ¹H NMR (400 MHz, MeOD-d₄) δ=7.92-7.79 (m, 1H), 7.37-7.28 (m, 2H), 7.27-7.14 (m, 1H), 6.61-6.25 (m, 1H), 5.45-5.18 (m, 1H), 4.76-4.61 (m, 1H), 4.61-4.43 (m, 2H), 4.35-4.20 (m, 3H), 4.15-3.98 (m, 1H), 3.54-3.41 (m, 1H), 3.30-3.17 (m, 3H), 3.11-2.95 (m, 1H), 2.33 (d, J=4.4 Hz, 2H), 2.16 (s, 1H), 2.06-1.96 (m, 2H), 1.95-1.83 (m, 1H), 1.42 (d, J=6.8 Hz, 3H); LCMS: (M+1=626.1).

Ex. 129 was prepared following General Method D, using I-2-39 in Step 1, and using PdCl₂(dtbpf) and Cs₂CO₃ (instead of cataCXium® A Pd G3 and K₃PO₄) in Step 3.

### Preparation of 4-[(9S)-10-(2,2-difluoroethyl)-4-fluoro-2-{[(2R,7aS)-2-fluorotetrahydro-1H-pyrrolizin-7a(5H)-yl]methoxy}-9-methyl-9,10-dihydro-8H-7-oxa-1,3,6,10-tetraazacyclohepta[1,2,3-de]naphthalen-5-yl]-5-ethyl-6-fluoronaphthalen-2-ol (Ex. 131)

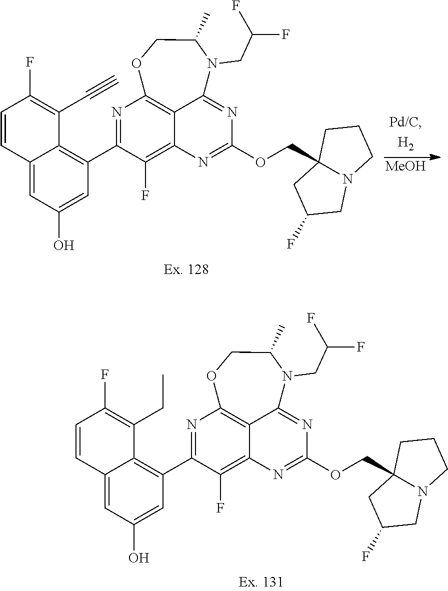 [SMILES: "MeOH" ; C#Cc1c(F)ccc2cc(O)cc(-c3nc4c5c(nc(OC[C@@]67CCCN6C[C@H](F)C7)nc5c3F)N(CC(F)F)[C@@H](C)CO4)c12 ; CCc1c(F)ccc2cc(O)cc(-c3nc4c5c(nc(OC[C@@]67CCCN6C[C@H](F)C7)nc5c3F)N(CC(F)F)[C@@H](C)CO4)c12]

To a solution of 4-[(12S)-13-(2,2-difluoroethyl)-6-fluoro-3-[[(2R,8S)-2-fluoro-1,2,3,5,6,7-hexahydropyrrolizin-8-yl]methoxy]-12-methyl-10-oxa-2,4,8,13-tetrazatricyclo[7.4.1.05,14]tetradeca-1,3,5,7,9 (14)-pentaen-7-yl]-5-ethynyl-6-fluoro-naphthalen-2-ol (80.0 mg, 0.127 mmol, 1 eq, Ex. 128) in MeOH (1 mL) was added Pd/C (10%, 100 mg) under N₂ atmosphere. The suspension was degassed and purged with H₂ thrice. The mixture was stirred under H₂ (15 psi) at 25° C. for 12 hr. On completion, the mixture was filtered to remove Pd/C. The residue purified by prep-HPLC (column: Waters Xbridge 150*25 mm*5 um; mobile phase: [water(NH₄HCO₃)-ACN]; gradient: 43%-73% B over 11 min) to give 4-[(12S)-13-(2,2-difluoroethyl)-6-fluoro-3-[[(2R,8S)-2-fluoro-1,2,3,5,6,7-hexahydropyrrolizin-8-yl]methoxy]-12-methyl-10-oxa-2,4,8,13-tetrazatricyclo[7.4.1.05,14]tetradeca-1,3,5,7,9 (14)-pentaen-7-yl]-5-ethyl-6-fluoro-naphthalen-2-ol (4.25 mg, 0.0067 mmol, 5.25% yield, 99.44% purity) as a white solid (Ex. 131). ¹H NMR (400 MHz, MeOD-d₄) δ=7.71-7.60 (m, 1H), 7.33-7.16 (m, 2H), 7.13-6.97 (m, 1H), 6.63-6.23 (m, 1H), 5.41-5.18 (m, 1H), 4.72-4.61 (m, 1H), 4.61-4.42 (m, 3H), 4.36-4.23 (m, 3H), 4.19-4.04 (m, 1H), 3.26-3.18 (m, 2H), 3.09-2.95 (m, 1H), 2.62-2.32 (m, 2H), 2.31-2.19 (m, 2H), 2.16-2.05 (m, 1H), 2.05-1.96 (m, 2H), 1.93-1.82 (m, 1H), 1.40 (d, J=6.8 Hz, 3H), 0.92-0.85 (m, 3H); LCMS: (M+1=630.2).

### Preparation of 5-ethyl-6-fluoro-4-(4-fluoro-2-{[(2R,7aS)-2-fluorotetrahydro-1H-pyrrolizin-7a(5H)-yl]methoxy}-10-methyl-8H,10H-spiro[7-oxa-1,3,6,10-tetraazacyclohepta[1,2,3-de]naphthalene-9,3′-oxolan]-5-yl)naphthalen-2-ol (Ex. 132)

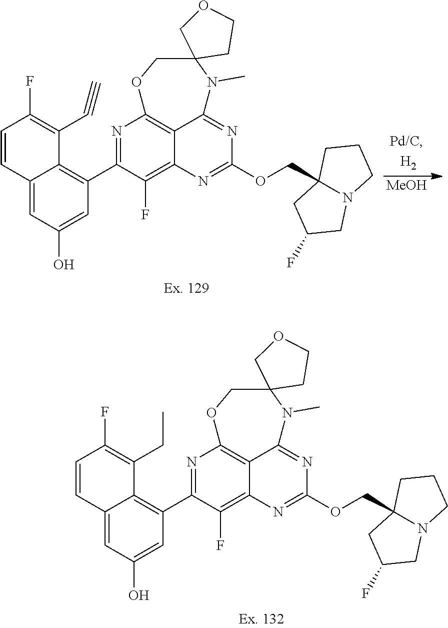 [SMILES: "MeOH" ; C#Cc1c(F)ccc2cc(O)cc(-c3nc4c5c(nc(OC[C@@]67CCCN6C[C@H](F)C7)nc5c3F)N(C)C3(CCOC3)CO4)c12 ; CCc1c(F)ccc2cc(O)cc(-c3nc4c5c(nc(OC[C@@]67CCCN6C[C@H](F)C7)nc5c3F)N(C)C3(CCOC3)CO4)c12]

A mixture of 5-ethynyl-6-fluoro-4-[6-fluoro-3-[[(2R,8S)-2-fluoro-1,2,3,5,6,7-hexahydropyrrolizin-8-yl]methoxy]-13-methyl-spiro[10-oxa-2,4,8,13-tetrazatricyclo[7.4.1.05,14]tetradeca-1,3,5,7,9 (14)-pentaene-12,3′-tetrahydrofuran]-7-yl]naphthalen-2-ol (100 mg, 0.161 mmol, 1 eq, Ex. 129), Pd/C (50.0 mg, 0.469 mmol, 10% purity, 2.90 eq) in MeOH (2 ml) was degassed and purged with H₂ 3 times, and then the mixture was stirred at 25° C. for 16 h under H₂ atmosphere. On completion, the mixture was filtered, and the filtrate was concentrated in vacuum to give a crude residue. The residue was purified by prep-HPLC (column: Waters Xbridge BEH C18 150*25 mm*5 um; mobile phase: [water(NH₄HCO₃)-ACN]; gradient: 37%-67% B over 10 min) to give 5-ethyl-6-fluoro-4-[6-fluoro-3-[[(2R,8S)-2-fluoro-1,2,3,5,6,7-hexahydropyrrolizin-8-yl]methoxy]-13-methyl-spiro[10-oxa-2,4,8,13-tetrazatricyclo[7.4.1.05,14]tetradeca-1,3,5,7,9 (14)-pentaene-12,3′-tetrahydrofuran]-7-yl]naphthalen-2-ol (1.6 mg, 0.0026 mmol, 1.59% yield, Ex. 132) as yellow solid. ¹H NMR (400 MHz, MeOD-d₄) δ=7.65 (dd, J=5.6, 9.2 Hz, 1H), 7.30-7.20 (m, 2H), 7.06 (t, J=2.0 Hz, 1H), 5.39-5.22 (m, 1H), 4.58 (s, 2H), 4.37-4.25 (m, 2H), 4.23-4.11 (m, 2H), 4.01-3.79 (m, 2H), 3.41 (d, J=1.6 Hz, 3H), 3.28-3.17 (m, 3H), 3.07-2.97 (m, 1H), 2.54-2.40 (m, 2H), 2.40-2.32 (m, 1H), 2.29-2.19 (m, 3H), 2.18-2.09 (m, 1H), 2.04-1.96 (m, 2H), 1.95-1.84 (m, 1H), 0.86 (q, J=6.8 Hz, 3H); LCMS: (M+1=622.2).

Ex. 133 was prepared following General Method D, using I-2-41 in Step 1, and using PdCl₂(dtbpf) and Cs₂CO₃ (instead of cataCXium® A Pd G3 and K₃PO₄) in Step 3.

The following examples were synthesized as indicated in the general methods and table above:

| Ex. # | Structure | ¹H NMR |
|---|---|---|
| 1 | 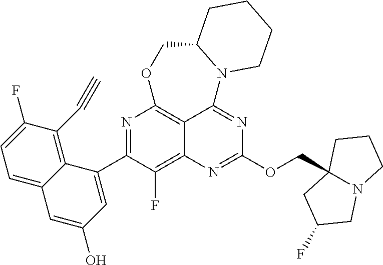 [SMILES: C#Cc1c(F)ccc2cc(O)cc(-c3nc4c5c(nc(OC[C@@]67CCCN6C[C@H](F)C7)nc5c3F)N3CCCC[C@H]3CO4)c12] | 1H NMR (400 MHz, DMSO-d6 ) δ = 10.29- 9.99-(m, 1H), 7.99-7.93 (m, 1H), 7.46 (t, J = 8.8 Hz, 1H), 7.37 (d, J = 2.4 Hz, 1H), 7.18-7.12 (m, 1H), 5.38-5.19 (m, 1H), 4.52- 4.38 (m, 2H), 4.06 (s, 2H), 4.01-3.91 (m, 2H), 3.13- 3.07 (m, 2H), 3.03 (br s, 2H), 2.87-2.79 (m, 1H), 2.15-2.05 (m, 2H), 2.03- 1.98 (m, 1H), 1.95-1.66 (m, 10H) |
| 2 | 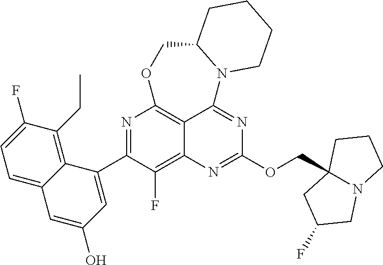 [SMILES: CCc1c(F)ccc2cc(O)cc(-c3nc4c5c(nc(OC[C@@]67CCCN6C[C@H](F)C7)nc5c3F)N3CCCC[C@H]3CO4)c12] | 1H NMR (400 MHz, DMSO-d6 ) δ = 7.74 (dd, J = 6.0, 9.0 Hz, 1H), 7.40-7.24 (m, 2H), 7.00 (dd, J = 2.6, 18.1 Hz, 1H), 5.40-5.05 (m, 2H), 4.57-4.40 (m, 2H), 4.14 (d, J = 10.4 Hz, 1H), 4.03-3.90 (m, 2H), 3.16- 2.95 (m, 5H), 2.90-2.77 (m, 1H), 2.35-2.22 (m, 2H), 2.16-2.11 (m, 1H), 2.00 (br s, 1H), 1.92-1.60 (m, 8H), 1.57-1.43 (m, 1H), 0.87- 0.73 (m, 3H) |
| 3 | 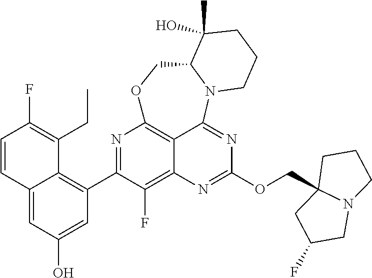 [SMILES: CCc1c(F)ccc2cc(O)cc(-c3nc4c5c(nc(OC[C@@]67CCCN6C[C@H](F)C7)nc5c3F)N3CCC[C@@](C)(O)[C@H]3CO4)c12] | 1H NMR (400 MHz, DMSO-d6 ) δ = 9.90 ( s, 1H), 7.77-7.70 (m, 1H), 7.33 ( t, J = 9.2 Hz, 1H), 7.29 (d, J = 2.4 Hz, 1H), 7.02-6.95 (m, 1H), 5.38- 5.19 (m, 1H), 5.10 (d, J = 11.6 Hz, 1H), 4.74-4.61 (m, 1H), 4.57-4.48 (m, 1H), 4.45 ( d, J = 19.2 Hz, 1H), 4.19-4.12 (m, 1H), 4.05- 3.98 (m, 1H), 3.87 ( s, 1H), 3.10 ( d, J = 7.6 Hz, 2H), 3.06-2.98 (m, 2H), 2.87- 2.78 (m, 1H), 2.45-2.35 (m, 1H), 2.33-2.23 (m, 1H), 2.17-2.10 (m, 1H), 2.07 ( s, 1H), 2.03-1.94 (m, 2H), 1.84 (d, J = 15.6 Hz, 1H), 1.80-1.73 (m, 2H), 1.72- 1.62 (m, 2H), 1.57 ( d, J = 12.4 Hz, 1H), 1.28-1.23 (m, 3H), 0.81-0.74 (m, 3H) |
| 5a/5b | 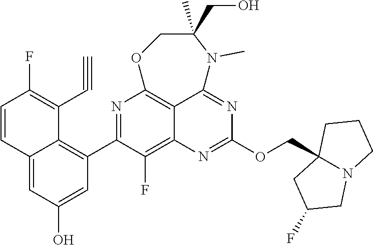 [SMILES: C#Cc1c(F)ccc2cc(O)cc(-c3nc4c5c(nc(OC[C@@]67CCCN6C[C@H](F)C7)nc5c3F)N(C)[C@](C)(CO)CO4)c12] 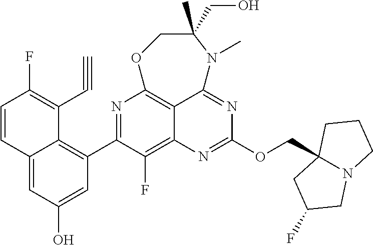 [SMILES: C#Cc1c(F)ccc2cc(O)cc(-c3nc4c5c(nc(OC[C@@]67CCCN6C[C@H](F)C7)nc5c3F)N(C)[C@@](C)(CO)CO4)c12] | ¹H NMR (400 MHz, MeOD- d₄) δ = 7.85 (dd, J = 5.6, 9.2 Hz, 1H), 7.36-7.28 (m, 2H), 7.25-7.18 (m, 1H), 5.65- 5.44 (m, 1H), 4.71 (d, J = 8.4 Hz, 1H), 4.67-4.63 (m, 2H), 4.53 (d, J = 4.0 Hz, 1H), 4.07-3.91 (m, 2H), 3.86 (dd, J = 2.4, 15.2 Hz, 2H), 3.77 (dd, J = 7.6, 12.0 Hz, 1H), 3.50-3.44 (m, 2H), 3.41 (s, 3H), 2.75-2.53 (m, 2H), 2.47-2.27 (m, 3H), 2.20-2.03 (m, 1H), 1.49 (d, J = 10.0 Hz, 3H) |
| 25 | 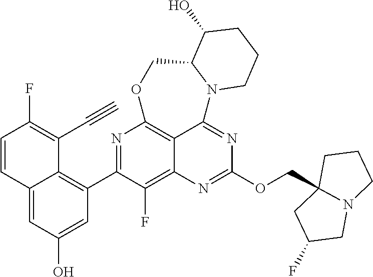 [SMILES: C#Cc1c(F)ccc2cc(O)cc(-c3nc4c5c(nc(OC[C@@]67CCCN6C[C@H](F)C7)nc5c3F)N3CCC[C@@H](O)[C@H]3CO4)c12] | 1H NMR (400 MHz, METHANOL-d4 ) δ = 7.92- 7.79 (m, 1H), 7.40-7.29 (m, 2H), 7.18 (br d, J = 5.4 Hz, 1H), 5.55-5.17 (m, 2H), 4.71-4.56 (m, 3H), 4.55- 4.45 (m, 1H), 4.44-4.34 (m, 1H), 4.23-4.08 (m, 2H), 3.69-3.43 (m, 4H), 3.26- 3.11 (m, 2H), 2.57-2.09 (m, 6H), 2.04-1.89 (m, 3H), 1.63 (s, 1H) |
| 27 | 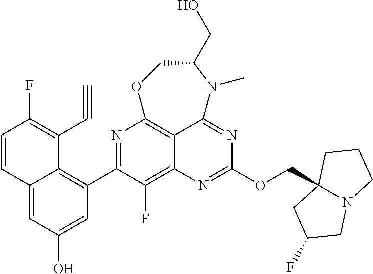 [SMILES: C#Cc1c(F)ccc2cc(O)cc(-c3nc4c5c(nc(OC[C@@]67CCCN6C[C@H](F)C7)nc5c3F)N(C)[C@@H](CO)CO4)c12] | 1H NMR (400 MHz, METHANOL-d4 ) δ = 7.87- 7.81 (m, 1H), 7.35-7.18 (m, 3H), 5.50-5.30 (m, 1H), 4.53-4.36 (m, 3H), 4.14- 4.04 (m, 1H), 3.98-3.89 (m, 1H), 3.87-3.78 (m, 1H), 3.58 (br d, J = 2.8 Hz, 1H), 3.56-3.43 (m, 6H), 3.41 (s, 1H), 3.22-3.13 (m, 1H), 2.53-2.32 (m, 2H), 2.32- 2.19 (m, 1H), 2.18-2.06 (m, 2H), 2.03-1.92 (m, 1H) |
| 28 | 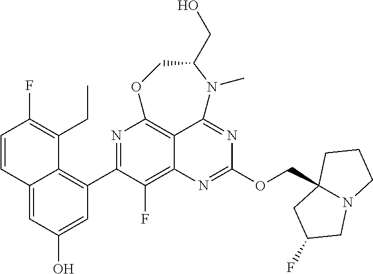 [SMILES: CCc1c(F)ccc2cc(O)cc(-c3nc4c5c(nc(OC[C@@]67CCCN6C[C@H](F)C7)nc5c3F)N(C)[C@@H](CO)CO4)c12] | 1H NMR (400 MHz, METHANOL-d4 ) δ = 7.70- 7.61 (m, 1H), 7.31-7.20 (m, 2H), 7.14-6.99 (m, 1H), 5.52-5.34 (m, 1H), 4.58- 4.42 (m, 3H), 4.15-4.07 (m, 1H), 3.96-3.87 (m, 1H), 3.87-3.76 (m, 1H), 3.73- 3.47 (m, 6H), 3.28-3.19 (m, 1H), 2.67-2.33 (m, 4H), 2.32-2.22 (m, 2H), 2.21- 2.10 (m, 2H), 2.09-1.97 (m, 1H), 0.93-0.80 (m, 3H) |
| 29 | 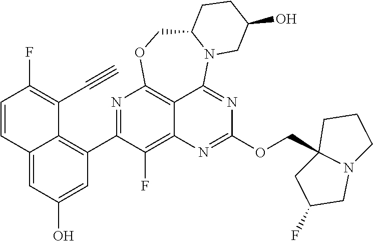 [SMILES: C#Cc1c(F)ccc2cc(O)cc(-c3nc4c5c(nc(OC[C@@]67CCCN6C[C@H](F)C7)nc5c3F)N3C[C@H](O)CC[C@H]3CO4)c12] | 1H NMR (400 MHz, DMSO-d6 ) δ = 10.13 (s, 1H), 7.99-7.92 (m, 1H), 7.45 (t, J = 9.2 Hz, 1H), 7.36 (d, J = 2.0 Hz, 1H), 7.18- 7.10 (m, 1H), 5.38-5.09 (m, 3H), 4.56-4.38 (m, 2H), 4.16-4.05 (m, 2H), 4.04- 3.97 (m, 1H), 3.94-3.83 (m, 1H), 3.64-3.46 (m, 1H), 3.10 (d, J = 9.2 Hz, 2H), 3.02 (s, 1H), 2.88-2.81 (m, 1H), 2.76 (t, J = 12.0 Hz, 1H), 2.13 (s, 1H), 2.10-2.08 (m, 1H), 2.06 (s, 1H), 2.01 (s, 1H), 1.87-1.82 (m, 2H), 1.81-1.76 (m, 2H), 1.73- 1.66 (m, 1H), 1.56 (d, J = 10.0 Hz, 1H) |
| 30 | 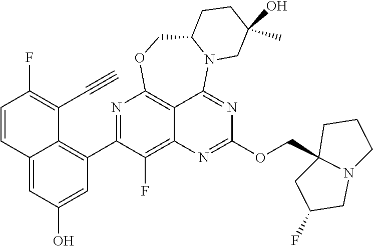 [SMILES: C#Cc1c(F)ccc2cc(O)cc(-c3nc4c5c(nc(OC[C@@]67CCCN6C[C@H](F)C7)nc5c3F)N3C[C@](C)(O)CC[C@H]3CO4)c12] | 1H NMR (400 MHz, DMSO-d6 ) δ = 10.16 (br d, J = 11.2 Hz, 1H), 7.99-7.93 (m, 1H), 7.48-7.42 (m, 1H), 7.37 (t, J = 2.4 Hz, 1H), 7.26- 7.11 (m, 1H), 5.40-5.16 (m, 1H), 5.06-4.83 (m, 1H), 4.53 (br s, 1H), 4.46 (d, J = 17.6 Hz, 1H), 4.17-4.12 (m, 1H), 3.93 (d, J = 10.4 Hz, 1H), 3.85-3.78 (m, 1H), 3.12-3.07 (m, 2H), 3.02 (br s, 2H), 2.87-2.81 (m, 1H), 2.14 (br d, J = 4.0 Hz, 1H), 2.05 (br s, 1H), 2.04-1.97 (m, 2H), 1.89-1.80 (m, 2H), 1.80-1.72 (m, 4H), 1.70- 1.59 (m, 2H), 1.18 (d, J = 10.0 Hz, 3H) |
| 31 | 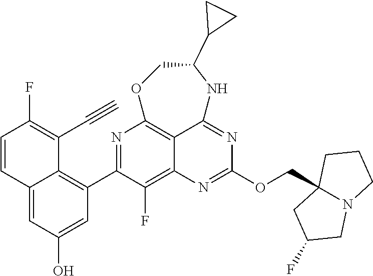 [SMILES: C#Cc1c(F)ccc2cc(O)cc(-c3nc4c5c(nc(OC[C@@]67CCCN6C[C@H](F)C7)nc5c3F)N[C@@H](C3CC3)CO4)c12] | 1H NMR (400 MHz, DMSO-d6 ) δ = 10.14 (d, J = 4.9 Hz, 1H), 9.41-9.25 (m, 1H), 7.96 (dd, J = 5.9, 9.1 Hz, 1H), 7.53-7.43 (m, 1H), 7.37 (d, J = 2.5 Hz, 1H), 7.16 (dd, J = 2.3, 13.2 Hz, 1H), 5.40-5.18 (m, 1H), 4.62-4.50 (m, 1H), 4.43 (dd, J = 7.1, 12.2 Hz, 1H), 4.15-4.09 (m, 1H), 4.08- 4.03 (m, 1H), 3.31 (s, 2H), 3.19 (br s, 4H), 2.92-2.81 (m, 1H), 2.62 (br s, 1H), 2.08-1.97 (m, 2H), 1.86- 1.78 (m, 2H), 1.17-1.05 (m, 1H), 0.62-0.51 (m, 2H), 0.50-0.38 (m, 2H) |
| 32 | 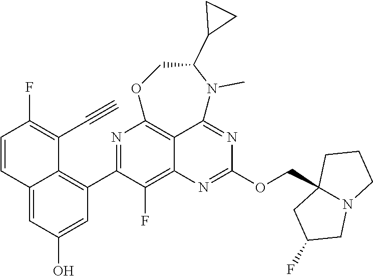 [SMILES: C#Cc1c(F)ccc2cc(O)cc(-c3nc4c5c(nc(OC[C@@]67CCCN6C[C@H](F)C7)nc5c3F)N(C)[C@@H](C3CC3)CO4)c12] | 1H NMR (400 MHz, DMSO-d6 ) δ = 10.13 (br d, J = 12.9 Hz, 1H), 8.00-7.93 (m, 1H), 7.46 (dt, J = 4.5, 9.0 Hz, 1H), 7.38 (d, J = 2.4 Hz, 1H), 7.12 (d, J = 2.5 Hz, 1H), 5.43-5.23 (m, 1H), 4.69-4.57 (m, 1H), 4.44- 4.37 (m, 1H), 4.28-4.17 (m, 1H), 4.16-4.07 (m, 1H), 4.07-3.99 (m, 1H), 3.42 (s, 3H), 3.40-3.38 (m, 1H), 3.24-3.11 (m, 3H), 2.97- 2.84 (m, 1H), 2.26-2.01 (m, 3H), 1.97-1.76 (m, 3H), 1.10-0.98 (m, 1H), 0.74- 0.52 (m, 3H), 0.39 (qd, J = 4.7, 9.4 Hz, 1H) |
| 33 | 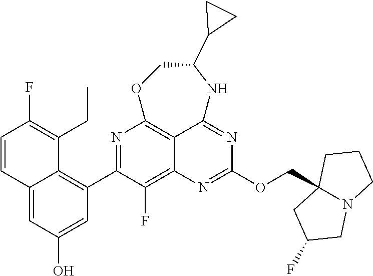 [SMILES: CCc1c(F)ccc2cc(O)cc(-c3nc4c5c(nc(OC[C@@]67CCCN6C[C@H](F)C7)nc5c3F)N[C@@H](C3CC3)CO4)c12] | 1H NMR (400 MHz, METHANOL-d4 ) δ = 7.66 (dd, J = 5.8, 9.1 Hz, 1H), 7.33-7.17 (m, 2H), 7.14- 6.98 (m, 1H), 5.53-5.36 (m, 1H), 4.69-4.57 (m, 2H), 4.53-4.43 (m, 2H), 3.76- 3.63 (m, 1H), 3.62-3.52 (m, 2H), 3.21-3.12 (m, 1H), 2.59-2.38 (m, 3H), 2.36- 2.16 (m, 4H), 2.09-1.98 (m, 1H), 1.23-1.12 (m, 1H), 0.90-0.83 (m, 3H), 0.78- 0.62 (m, 2H), 0.59-0.42 (m, 2H) |
| 35 | 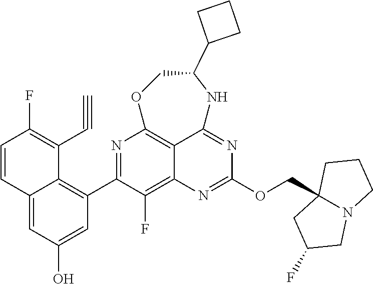 [SMILES: C#Cc1c(F)ccc2cc(O)cc(-c3nc4c5c(nc(OC[C@@]67CCCN6C[C@H](F)C7)nc5c3F)N[C@@H](C3CCC3)CO4)c12] | 1H NMR (400 MHz, DMSO-d6 ) δ = 10.26-9.98 (m, 1H), 9.36 (t, J = 5.2 Hz, 1H), 7.95 (dd, J = 5.6, 9.2 Hz, 1H), 7.45 (t, J = 9.2 Hz, 1H), 7.36 (d, J = 2.4 Hz, 1H), 7.14 (dd, J = 2.4, 14.4 Hz, 1H), 5.16 (s, 1H), 4.48 (dd, J = 4.0, 12.4 Hz, 1H), 4.31 (dd, J = 3.2, 12.4 Hz, 1H), 4.16-3.96 (m, 3H), 3.71-3.59 (m, 1H), 3.14- 3.07 (m, 2H), 3.06-3.00 (m, 1H), 2.88-2.79 (m, 1H), 2.14-2.08 (m, 1H), 2.06- 1.74 (m, 12H) |
| 39 | 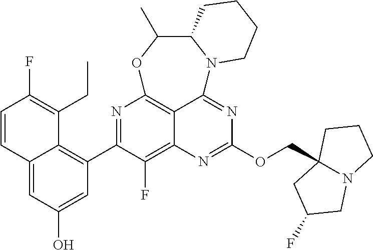 [SMILES: CCc1c(F)ccc2cc(O)cc(-c3nc4c5c(nc(OC[C@@]67CCCN6C[C@H](F)C7)nc5c3F)N3CCCC[C@H]3C(C)O4)c12] | 1H NMR (400 MHz, METHANOL-d4 ) δ = 7.71- 7.60 (m, 1H), 7.34-7.18 (m, 2H), 7.09-6.99 (m, 1H), 5.48-5.32 (m, 1H), 5.30- 5.21 (m, 1H), 4.63-4.51 (m, 2H), 4.46-4.41 (m, 1H), 4.36-4.31 (m, 1H), 3.96- 3.89 (m, 1H), 3.47-3.40 (m, 2H), 3.19-3.11 (m, 2H), 2.54-2.43 (m, 2H), 2.40- 2.30 (m, 2H), 2.29-2.18 (m, 2H), 2.14-2.07 (m, 2H), 2.01-1.95 (m, 2H), 1.88- 1.81 (m, 2H), 1.65-1.57 (m, 1H), 1.52-1.43 (m, 3H), 0.95-0.80 (m, 3H) |
| 40 | 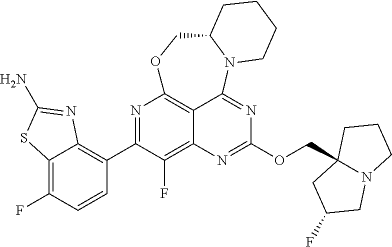 [SMILES: Nc1nc2c(-c3nc4c5c(nc(OC[C@@]67CCCN6C[C@H](F)C7)nc5c3F)N3CCCC[C@H]3CO4)ccc(F)c2s1] | 1H NMR (400 MHz, DMSO-d6 ) δ = 7.94-7.86 (m, 2H), 7.34 (dd, J = 5.6, 8.4 Hz, 1H), 7.03 (t, J = 8.8 Hz, 1H), 5.37-5.19 (m, 1H), 5.16-5.07 (m, 1H), 4.52- 4.39 (m, 2H), 4.16-3.95 (m, 2H), 3.93-3.85 (m, 1H), 3.11-3.06 (m, 2H), 3.05- 2.99 (m, 2H), 2.85-2.79 (m, 1H), 2.15-2.10 (m, 1H), 2.05 (br s, 2H), 1.91-1.66 (m, 8H), 1.53-1.39 (m, 1H) |
| 41 | 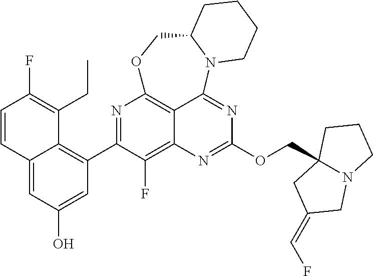 [SMILES: CCc1c(F)ccc2cc(O)cc(-c3nc4c5c(nc(OC[C@@]67CCCN6C/C(=C\F)C7)nc5c3F)N3CCCC[C@H]3CO4)c12] | 1H NMR (400 MHz, DMSO-d6 ) δ = 7.67 (s, 1H), 7.37-7.33 (m, 1H), 7.30 (br s, 1H), 7.06-6.90 (m, 1H), 5.21-5.03 (m, 1H), 4.59- 4.40 (m, 2H), 4.16-4.00 (m, 2H), 3.98-3.86 (m, 1H), 3.04-2.92 (m, 2H), 2.42- 2.25 (m, 4H), 2.10-1.95 (m, 2H), 1.93-1.58 (m, 10H), 1.54-1.16 (m, 3H), 0.87- 0.73 (m, 3H) |
| 43 | 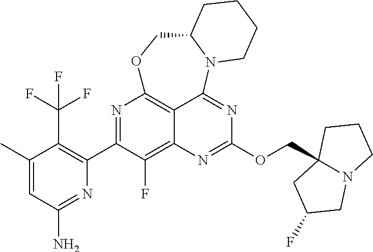 [SMILES: Cc1cc(N)nc(-c2nc3c4c(nc(OC[C@@]56CCCN5C[C@H](F)C6)nc4c2F)N2CCCC[C@H]2CO3)c1C(F)(F)F] | 1H NMR (400 MHz, DMSO-d6 ) δ = 6.80-6.76 (m, 2H), 6.47 (s, 1H), 5.29 (s, 1H), 5.14-5.06 (m, 1H), 4.46-4.41 (m, 2H), 4.14- 4.09 (m, 1H), 4.00-3.95 (m, 1H), 3.90 (br d, J = 9.2 Hz, 1H), 3.12-3.06 (m, 3H), 3.01 (s, 2H), 2.86-2.77 (m, 2H), 2.68-2.66 (m, 1H), 2.36-2.34 (m, 3H), 2.14- 2.10 (m, 1H), 2.04 (br d, J = 2.4 Hz, 1H), 2.00 (br d, J = 4.4 Hz, 1H), 1.86-1.82 (m, 2H), 1.79-1.74 (m, 3H), 1.53-1.42 (m, 1H) |
| 44 | 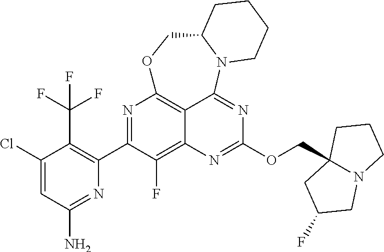 [SMILES: Nc1cc(Cl)c(C(F)(F)F)c(-c2nc3c4c(nc(OC[C@@]56CCCN5C[C@H](F)C6)nc4c2F)N2CCCC[C@H]2CO3)n1] | 1H NMR (400 MHz, DMSO-d6 ) δ = 9.61 (s, 1H), 8.18-8.13 (m, 1H), 7.89- 7.85 (m, 1H), 7.76-7.72 (m, 1H), 5.21 (br s, 1H), 4.52- 4.39 (m, 2H), 4.14-3.97 (m, 1H), 3.99-3.93 (m, 1H), 3.91-3.82 (m, 1H), 3.12- 3.06 (m, 2H), 3.03-2.93 (m, 2H), 2.67 (s, 2H), 2.33 (s, 2H), 2.06-1.98 (m, 2H), 1.81 (br s, 2H), 1.79-1.65 (m, 4H), 1.42-1.22 (m, 2H) |
| 45 | 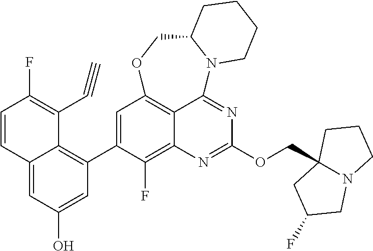 [SMILES: C#Cc1c(F)ccc2cc(O)cc(-c3cc4c5c(nc(OC[C@@]67CCCN6C[C@H](F)C7)nc5c3F)N3CCCC[C@H]3CO4)c12] | 1H NMR (400 MHz, METHANOL-d4 ) δ = 7.82 (dd, J = 5.6, 9.2 Hz, 1H), 7.35-7.25 (m, 2H), 7.08 (t, J = 3.0 Hz, 1H), 6.81 (dd, J = 1.8, 6.4 Hz, 1H), 5.53-5.24 (m, 2H), 4.58-4.29 (m, 4H), 3.93 (br d, J = 6.0 Hz, 1H), 3.75-3.49 (m, 3H), 3.39 (d, J = 3.6 Hz, 1H), 3.26 (br d, J = 5.2 Hz, 1H), 3.06 (br t, J = 12.4 Hz, 1H), 2.58-2.38 (m, 2H), 2.36-2.25 (m, 1H), 2.24-2.09 (m, 2H), 2.07- 1.94 (m, 2H), 1.93-1.73 (m, 4H), 1.69-1.58 (m, 1H) |
| 46 | 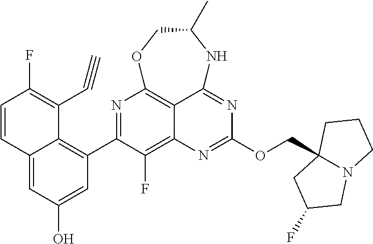 [SMILES: C#Cc1c(F)ccc2cc(O)cc(-c3nc4c5c(nc(OC[C@@]67CCCN6C[C@H](F)C7)nc5c3F)N[C@@H](C)CO4)c12] | 1H NMR (400 MHz, METHANOL-d4 ) δ = 7.87- 7.81 (m, 1H), 7.34-7.28 (m, 2H), 7.21-7.18 (m, 1H), 5.31 (br s, 1H), 4.61-4.55 (m, 2H), 4.51-4.33 (m, 4H), 4.10-4.01 (m, 1H), 3.52- 3.47 (m, 2H), 3.15 (br d, J = 4.4 Hz, 1H), 2.46-2.28 (m, 2H), 2.20-1.93 (m, 4H), 1.41 (d, J = 6.8 Hz, 3H) |
| 47 | 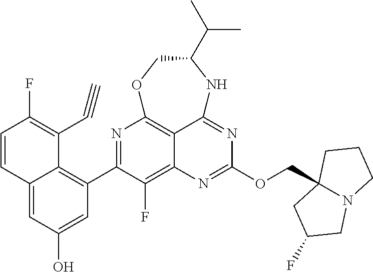 [SMILES: C#Cc1c(F)ccc2cc(O)cc(-c3nc4c5c(nc(OC[C@@]67CCCN6C[C@H](F)C7)nc5c3F)N[C@@H](C(C)C)CO4)c12] | 1H NMR (400 MHz, DMSO-d6 ) δ = 10.28-9.99 (m, 1H), 9.42-9.37 (m, 1H), 7.98-7.92 (m, 1H), 7.50- 7.41 (m, 1H), 7.39-7.32 (m, 1H), 7.20-7.10 (m, 1H), 5.39-5.16 (m, 1H), 4.64- 4.52 (m, 1H), 4.41-4.34 (m, 1H), 4.16-3.94 (m, 3H), 3.20-2.96 (m, 4H), 2.88- 2.80 (m, 1H), 2.15-1.67 (m, 7H), 1.09-0.93 (m, 6H) |
| 48 | 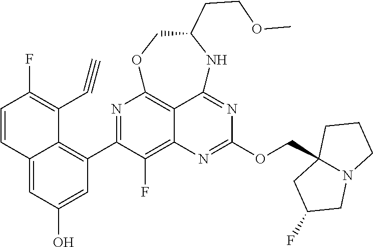 [SMILES: C#Cc1c(F)ccc2cc(O)cc(-c3nc4c5c(nc(OC[C@@]67CCCN6C[C@H](F)C7)nc5c3F)N[C@@H](CCOC)CO4)c12] | 1H NMR (400 MHz, METHANOL-d4 ) δ = 7.87- 7.81 (m, 1H), 7.34 (d, J = 2.4 Hz, 1H), 7.32-7.28 (m, 1H), 7.26-7.21 (m, 1H), 5.53- 5.32 (m, 1H), 4.66-4.53 (m, 3H), 4.51-4.39 (m, 2H), 4.07 (br s, 1H), 3.63 (br t, J = 5.6 Hz, 2H), 3.60-3.52 (m, 3H), 3.48 (s, 1H), 3.40 (d, J = 1.2 Hz, 3H), 3.27- 3.23 (m, 1H), 2.56-2.36 (m, 2H), 2.27-2.22 (m, 1H), 2.21-2.13 (m, 2H), 2.07- 1.96 (m, 3H) |
| 50 | 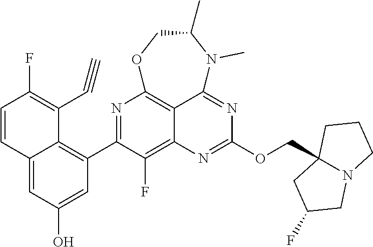 [SMILES: C#Cc1c(F)ccc2cc(O)cc(-c3nc4c5c(nc(OC[C@@]67CCCN6C[C@H](F)C7)nc5c3F)N(C)[C@@H](C)CO4)c12] | 1H NMR (400 MHz, METHANOL-d4 ) δ = 7.87- 7.82 (m, 1H), 7.88-7.79 (m, 1H), 7.37-7.28 (m, 2H), 7.28-7.15 (m, 1H), 5.53- 5.31 (m, 1H), 4.67-4.58 (m, 1H), 4.57-4.42 (m, 3H), 4.22-4.11 (m, 1H), 3.72- 3.50 (m, 4H), 3.50-3.47 (m, 3H), 3.27-3.19 (m, 1H), 2.59-2.35 (m, 2H), 2.31- 2.23 (m, 1H), 2.22-2.13 (m, 2H), 2.07-1.93 (m, 1H), 1.46-1.40 (m, 3H) |
| 51 | 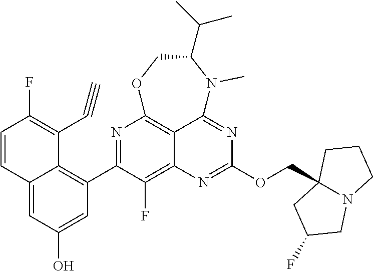 [SMILES: C#Cc1c(F)ccc2cc(O)cc(-c3nc4c5c(nc(OC[C@@]67CCCN6C[C@H](F)C7)nc5c3F)N(C)[C@@H](C(C)C)CO4)c12] | 1H NMR (400 MHz, METHANOL-d4 ) δ = 7.88- 7.81 (m, 1H), 7.35-7.27 (m, 2H), 7.26-7.13 (m, 1H), 5.43-5.28 (m, 1H), 4.61- 4.56 (m, 1H), 4.48-4.38 (m, 2H), 4.35-4.27 (m, 1H), 3.72 (br dd, J = 4.8, 10.1 Hz, 1H), 3.53 (s, 3H), 3.47-3.45 (m, 1H), 3.38-3.35 (m, 2H), 3.26 (s, 1H), 3.15-3.06 (m, 1H), 2.38-2.25 (m, 2H), 2.25-2.14 (m, 2H), 2.11- 2.05 (m, 2H), 1.99-1.87 (m, 1H), 1.16-1.06 (m, 6H) |
| 52 | 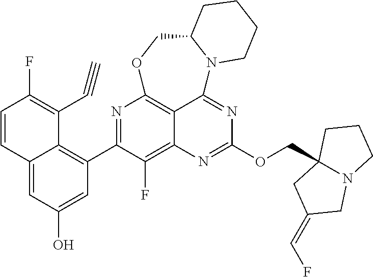 [SMILES: C#Cc1c(F)ccc2cc(O)cc(-c3nc4c5c(nc(OC[C@@]67CCCN6C/C(=C\F)C7)nc5c3F)N3CCCC[C@H]3CO4)c12] | 1H NMR (400 MHz, DMSO-d6 ) δ = 10.16- 10.09 (m, 1H), 8.01-7.91 (m, 1H), 7.49-7.42 (m, 1H), 7.40-7.34 (m, 1H), 7.19- 7.08 (m, 1H), 6.89-6.60 (m, 1H), 5.21-5.07 (m, 1H), 4.56-4.37 (m, 2H), 4.12- 4.07 (m, 1H), 4.02 (br s, 2H), 4.01-3.89 (m, 2H), 3.75-3.67 (m, 1H), 3.07- 2.97 (m, 2H), 2.37-2.28 (m, 1H), 2.02-1.63 (m, 10H), 1.60-1.27 (m, 2H) |
| 53 | 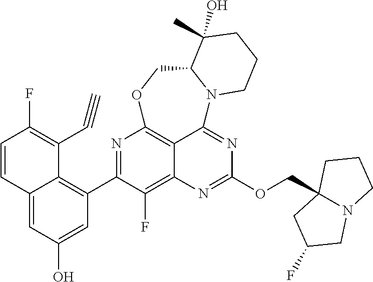 [SMILES: C#Cc1c(F)ccc2cc(O)cc(-c3nc4c5c(nc(OC[C@@]67CCCN6C[C@H](F)C7)nc5c3F)N3CCC[C@@](C)(O)[C@H]3CO4)c12] | 1H NMR (400 MHz, DMSO-d6 ) δ = 10.15- 10.07 (m, 1H), 7.99-7.90 (m, 1H), 7.45 (t, J = 8.8 Hz, 1H), 7.36 (s, 1H), 7.17-7.08 (m, 1H), 5.38-5.20 (m, 1H), 5.18-5.04 (m, 1H), 4.74- 4.60 (m, 1H), 4.59-4.45 (m, 1H), 4.44-4.36 (m, 1H), 4.13 ( t, J = 10.0 Hz, 1H), 4.06-3.93 (m, 2H), 3.91- 3.83 (m, 1H), 3.10 ( d, J = 8.4 Hz, 2H), 3.05-2.98 (m, 2H), 2.88-2.79 (m, 1H), 2.16-2.05 (m, 2H), 2.02- 1.93 (m, 2H), 1.84 ( d, J = 15.2 Hz, 1H), 1.80-1.74 (m, 2H), 1.73-1.64 (m, 2H), 1.57 ( d, J = 12.0 Hz, 1H), 1.28 ( d, J = 3.2 Hz, 3H) |
| 56 | 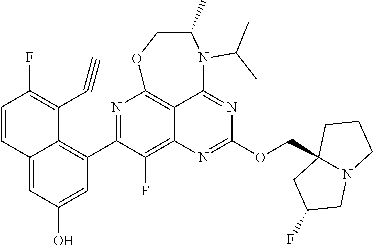 [SMILES: C#Cc1c(F)ccc2cc(O)cc(-c3nc4c5c(nc(OC[C@@]67CCCN6C[C@H](F)C7)nc5c3F)N(C(C)C)[C@@H](C)CO4)c12] | ¹H NMR (400 MHz, MeOD- d₄) δ = 7.89-7.80 (m, 1H), 7.36-7.28 (m, 2H), 7.26- 7.14 (m, 1H), 5.79-5.62 (m, 1H), 5.49-5.27 (m, 1H), 4.70-4.66 (m, 1H), 4.60- 4.54 (m, 1H), 4.44-4.27 (m, 4H), 3.53-3.42 (m, 3H), 3.19-3.12 (m, 1H), 2.51- 2.30 (m, 2H), 2.28-2.18 (m, 1H), 2.17-2.06 (m, 2H), 2.02-1.91 (m, 1H), 1.45- 1.40 (m, 3H), 1.33 (br s, 6H). |
| 58 | 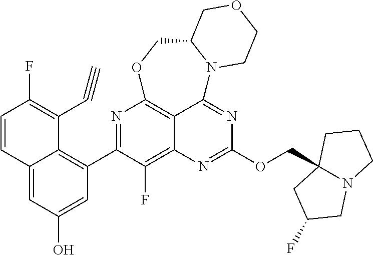 [SMILES: C#Cc1c(F)ccc2cc(O)cc(-c3nc4c5c(nc(OC[C@@]67CCCN6C[C@H](F)C7)nc5c3F)N3CCOC[C@H]3CO4)c12] | 1H NMR (400 MHz, METHANOL-d4 ) δ = 8.50- 8.37 (m, 1H), 7.85 (dd, J = 6.0, 9.0 Hz, 1H), 7.37-7.29 (m, 2H), 7.20 (br s, 1H), 5.56-5.39 (m, 1H), 5.26- 5.15 (m, 2H), 4.61-4.49 (m, 4H), 4.26-4.18 (m, 1H), 4.13-4.01 (m, 2H), 3.66 (br t, J = 11.2 Hz, 4H), 3.47- 3.36 (m, 2H), 2.66 (s, 1H), 2.62-2.43 (m, 2H), 2.37- 2.28 (m, 1H), 2.26-2.16 (m, 2H), 2.12-2.00 (m, 1H) |
| 59 | 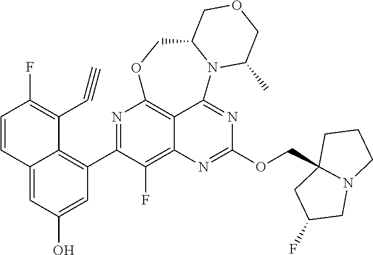 [SMILES: C#Cc1c(F)ccc2cc(O)cc(-c3nc4c5c(nc(OC[C@@]67CCCN6C[C@H](F)C7)nc5c3F)N3[C@@H](COC[C@@H]3C)CO4)c12] | 1H NMR (400 MHz, DMSO-d6 ) δ = 10.30-9.98 (m, 1H), 7.99-7.92 (m, 1H), 7.49-7.42 (m, 1H), 7.37 (d, J = 2.0 Hz, 1H), 7.24-7.05 (m, 1H), 5.43-5.14 (m, 1H), 4.75-4.54 (m, 1H), 4.53- 4.34 (m, 2H), 4.30-4.22 (m, 1H), 4.17-4.07 (m, 3H), 4.07-4.00 (m, 2H), 3.99- 3.85 (m, 1H), 3.83-3.74 (m, 1H), 3.10 (d, J = 5.6 Hz, 2H), 3.02 (s, 1H), 2.89-2.80 (m, 1H), 2.54 (s, 1H), 2.13 (s, 1H), 2.06 (s, 1H), 2.03- 1.95 (m, 1H), 1.85 (d, J = 14.5 Hz, 1H), 1.81-1.77 (m, 1H), 1.59-1.43 (m, 3H) |
| 74 | 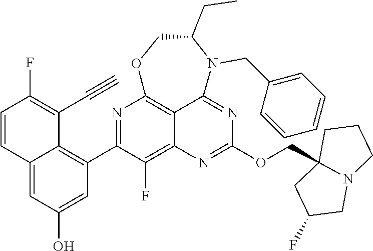 [SMILES: C#Cc1c(F)ccc2cc(O)cc(-c3nc4c5c(nc(OC[C@@]67CCCN6C[C@H](F)C7)nc5c3F)N(Cc3ccccc3)[C@@H](CC)CO4)c12] | 1H NMR (400 MHz, METHANOL-d4 ) δ = 7.90- 7.80 (m, 1H), 7.43-7.16 (m, 8H), 5.67-5.49 (m, 1H), 5.38-5.18 (m, 2H), 4.67- 4.48 (m, 4H), 4.24-4.15 (m, 2H), 4.11-4.02 (m, 1H), 3.62-3.45 (m, 2H), 3.12- 2.99 (m, 1H), 2.36-1.92 (m, 6H), 1.87-1.72 (m, 1H), 1.48-1.40 (m, 3H) |
| 75 | 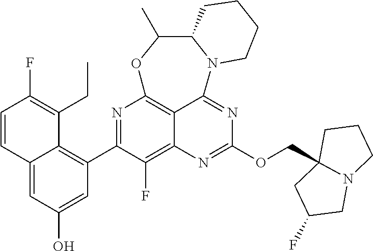 [SMILES: CCc1c(F)ccc2cc(O)cc(-c3nc4c5c(nc(OC[C@@]67CCCN6C[C@H](F)C7)nc5c3F)N3CCCC[C@H]3C(C)O4)c12] | 1H NMR (400 MHz, METHANOL-d4 ) δ = 7.71- 7.60 (m, 1H), 7.34-7.18 (m, 2H), 7.09-6.99 (m, 1H), 5.48-5.32 (m, 1H), 5.30- 5.21 (m, 1H), 4.63-4.51 (m, 2H), 4.46-4.41 (m, 1H), 4.36-4.31 (m, 1H), 3.96- 3.89 (m, 1H), 3.47-3.40 (m, 2H), 3.19-3.11 (m, 2H), 2.54-2.43 (m, 2H), 2.40- 2.30 (m, 2H), 2.29-2.18 (m, 2H), 2.14-2.07 (m, 2H), 2.01-1.95 (m, 2H), 1.88- 1.81 (m, 2H), 1.65-1.57 (m, 1H), 1.52-1.43 (m, 3H), 0.95-0.80 (m, 3H) |
| 76 | 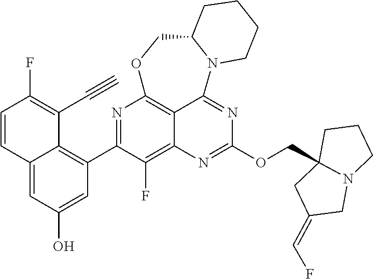 [SMILES: C#Cc1c(F)ccc2cc(O)cc(-c3nc4c5c(nc(OC[C@@]67CCCN6C/C(=C\F)C7)nc5c3F)N3CCCC[C@H]3CO4)c12] | 1H NMR (400 MHz, DMSO-d6 ) δ = 7.98-7.91 (m, 1H), 7.49-7.41 (m, 1H), 7.36 (d, J = 2.4 Hz, 1H), 7.14 (dd, J = 2.4, 13.8 Hz, 1H), 6.77-6.67 (m, 1H), 4.12-4.06 (m, 2H), 4.06- 3.99 (m, 2H), 3.99-3.90 (m, 2H), 3.20-3.16 (m, 2H), 3.09-3.00 (m, 2H), 2.69- 2.65 (m, 1H), 2.33 (s, 2H), 2.02-1.97 (m, 1H), 1.96- 1.64 (m, 10H) |
| 77 | 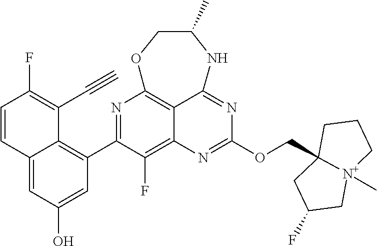 [SMILES: C#Cc1c(F)ccc2cc(O)cc(-c3nc4c5c(nc(OC[C@@]67CCC[N+]6(C)C[C@H](F)C7)nc5c3F)N[C@@H](C)CO4)c12] | 1H NMR (400 MHz, METHANOL-d4 ) δ = 7.88- 7.83 (m, 1H), 7.35-7.30 (m, 2H), 7.22-7.19 (m, 1H), 5.70-5.49 (m, 1H), 4.61- 4.59 (m, 4H), 4.53-4.43 (m, 2H), 4.34-4.16 (m, 2H), 4.11-4.01 (m, 2H), 3.92- 3.83 (m, 1H), 3.54-3.47 (m, 1H), 3.36-3.35 (m, 1H), 3.05-2.89 (m, 1H), 2.70- 2.58 (m, 1H), 2.32 (br s, 4H), 1.46-1.41 (m, 3H) |
| 78 | 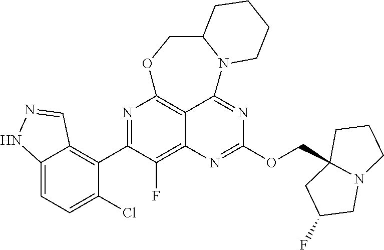 [SMILES: Fc1c(-c2c(Cl)ccc3[nH]ncc23)nc2c3c(nc(OC[C@@]45CCCN4C[C@H](F)C5)nc13)N1CCCCC1CO2] | 1H NMR (400 MHz, DMSO-d6 ) δ = 13.43 ( s, 1H), 7.82 ( s, 1H), 7.70 (d, J = 8.8 Hz, 1H), 7.51 (d, J = 8.8 Hz, 1H), 5.38-5.19 (m, 1H), 5.16-5.10 (m, 1H), 4.56-4.44 (m, 2H), 4.15 (d, J = 10.4 Hz, 1H), 4.01 (d, J = 10.4 Hz, 1H), 3.96-3.89 (m, 1H), 3.12-3.07 (m, 2H), 3.05-3.01 (m, 2H), 2.87- 2.79 (m, 1H), 2.13 (d, J = 7.2 Hz, 1H), 2.06 (br d, J = 2.0 Hz, 1H), 2.00 ( s, 1H), 1.93- 1.81 (m, 4H), 1.80-1.68 (m, 5H) |
| 79 | 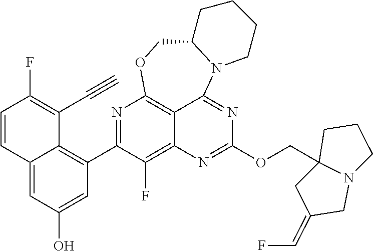 [SMILES: C#Cc1c(F)ccc2cc(O)cc(-c3nc4c5c(nc(OCC67CCCN6C/C(=C/F)C7)nc5c3F)N3CCCC[C@H]3CO4)c12] | 1H NMR (400 MHz, DMSO-d6 ) δ = 10.17- 10.09 (m, 1H), 7.96 (dd, J = 6.4, 9.1 Hz, 1H), 7.50-7.42 (m, 1H), 7.40-7.34 (m, 1H), 7.18-7.10 (m, 1H), 6.95- 6.70 (m, 1H), 4.49 (br d, J = 6.0 Hz, 2H), 4.12-4.08 (m, 1H), 4.07-4.03 (m, 1H), 4.02-3.88 (m, 2H), 3.05- 2.94 (m, 2H), 2.67 (br d, J = 1.2 Hz, 2H), 2.42-2.32 (m, 2H), 2.06-1.65 (m, 10H), 1.64-1.33 (m, 2H) |
| 80 | 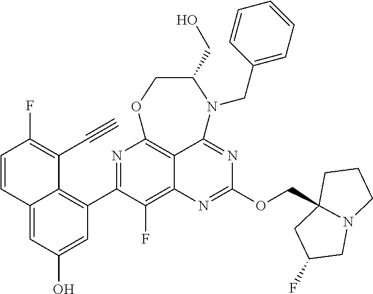 [SMILES: C#Cc1c(F)ccc2cc(O)cc(-c3nc4c5c(nc(OC[C@@]67CCCN6C[C@H](F)C7)nc5c3F)N(Cc3ccccc3)[C@@H](CO)CO4)c12] | 1H NMR (400 MHz, DMSO-d6 ) δ = 7.99-7.91 (m, 1H), 7.50-7.42 (m, 1H), 7.41-7.26 (m, 6H), 7.22- 7.09 (m, 1H), 5.79-5.51 (m, 1H), 5.35-5.04 (m, 2H), 4.86-4.61 (m, 2H), 4.49- 4.37 (m, 1H), 4.02-3.86 (m, 4H), 3.77-3.69 (m, 2H), 3.03-2.73 (m, 5H), 2.04- 1.85 (m, 3H), 1.82-1.57 (m, 3H) |
| 81 | 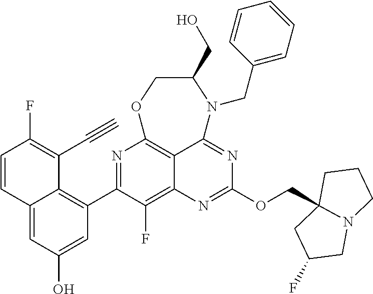 [SMILES: C#Cc1c(F)ccc2cc(O)cc(-c3nc4c5c(nc(OC[C@@]67CCCN6C[C@H](F)C7)nc5c3F)N(Cc3ccccc3)[C@H](CO)CO4)c12] | 1H NMR (400 MHz, DMSO-d6 ) δ = 8.01-7.91 (m, 1H), 7.46 (br t, J = 9.0 Hz, 1H), 7.42-7.27 (m, 6H), 7.23-7.09 (m, 1H), 5.78- 5.49 (m, 1H), 5.38-5.04 (m, 2H), 4.86-4.72 (m, 1H), 4.66-4.42 (m, 1H), 4.00- 3.85 (m, 4H), 3.79-3.70 (m, 2H), 3.02-2.74 (m, 5H), 2.08-1.83 (m, 3H), 1.81- 1.55 (m, 3H) |
| 82 | 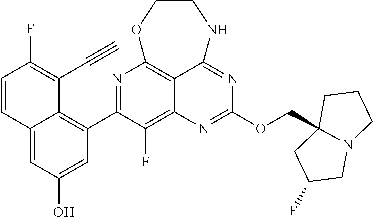 [SMILES: C#Cc1c(F)ccc2cc(O)cc(-c3nc4c5c(nc(OC[C@@]67CCCN6C[C@H](F)C7)nc5c3F)NCCO4)c12] | 1H NMR (400 MHz, DMSO-d6 ) δ = 9.21-9.13 (m, 1H), 8.18 (s, 1H), 7.95 (dd, J = 6.0, 9.2 Hz, 1H), 7.45 (t, J = 9.2 Hz, 1H), 7.36 (d, J = 2.4 Hz, 1H), 7.14 (d, J = 2.4 Hz, 1H), 5.37-5.16 (m, 1H), 4.49 (br s, 2H), 4.12-4.05 (m, 2H), 4.02- 3.95 (m, 1H), 3.69 (br d, J = 4.0 Hz, 2H), 3.11-3.07 (m, 2H), 3.04-2.97 (m, 2H), 2.86-2.79 (m, 1H), 2.13- 2.09 (m, 1H), 2.03 (br d, J = 2.0 Hz, 1H), 2.00-1.94 (m, 1H), 1.86-1.81 (m, 1H), 1.79-1.74 (m, 1H) |
| 83 | 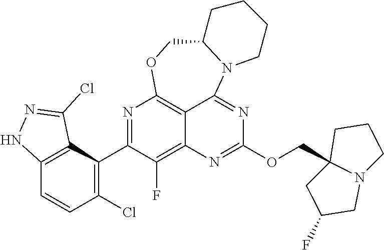 | 1H NMR (400 MHz, DMSO-d6 ) δ = 13.87- 13.49 (m, 1H), 7.74 (d, J = 9.0 Hz, 1H), 7.65-7.59 (m, 1H), 5.41-5.19 (m, 1H), 5.14-5.08 (m, 1H), 4.57- 4.39 (m, 2H), 4.13 (d, J = 10.3 Hz, 1H), 4.00 (d, J = 10.4 Hz, 1H), 3.97-3.92 (m, 1H), 3.13-3.06 (m, 2H), 3.06-3.00 (m, 2H), 2.86- 2.79 (m, 1H), 2.15-2.11 (m, 1H), 2.05 ( d, J = 2.4 Hz, 1H), 2.02-1.97 (m, 1H), 1.94-1.73 (m, 7H), 1.71- 1.64 (m, 1H), 1.57-1.44 (m, 1H) |
| 84 | 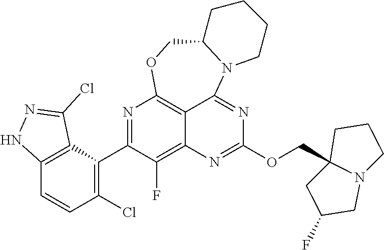 | 1H NMR (400 MHz, DMSO-d6 ) δ = 13.87- 13.53 (m, 1H), 7.74 (d, J = 9.2 Hz, 1H), 7.65-7.58 (m, 1H), 5.40-5.19 (m, 1H), 5.12 (d, J = 12.6 Hz, 1H), 4.57-4.43 (m, 2H), 4.13 (d, J = 10.4 Hz, 1H), 4.00 (d, J = 10.4 Hz, 1H), 3.97-3.91 (m, 1H), 3.12-3.06 (m, 2H), 3.06-3.00 (m, 2H), 2.87- 2.78 (m, 1H), 2.15-2.11 (m, 1H), 2.06 (s, 1H), 2.01 ( d, J = 4.4 Hz, 1H), 1.96-1.80 (m, 4H), 1.80-1.73 (m, 3H), 1.72-1.65 (m, 1H), 1.57- 1.45 (m, 1H) |
| 85 | 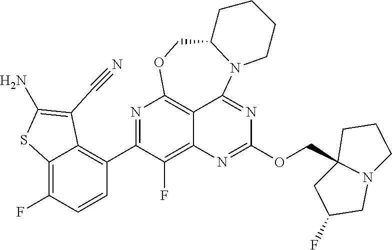 [SMILES: N#Cc1c(N)sc2c(F)ccc(-c3nc4c5c(nc(OC[C@@]67CCCN6C[C@H](F)C7)nc5c3F)N3CCCC[C@H]3CO4)c12] | 1H NMR (400 MHz, METHANOL-d4 ) δ = 7.42- 7.34 (m, 1H), 7.07-6.99 (m, 1H), 5.50-5.37 (m, 1H), 5.37-5.31 (m, 1H), 5.15- 5.05 (m, 1H), 4.54-4.51 (m, 2H), 4.45-4.34 (m, 2H), 4.03-3.93 (m, 1H), 3.49- 3.44 (m, 2H), 3.21-3.10 (m, 2H), 2.43-2.30 (m, 2H), 2.26-2.20 (m, 1H), 2.16- 2.09 (m, 2H), 2.06-1.91 (m, 4H), 1.88-1.83 (m, 2H), 1.70-1.54 (m, 1H) |
| 86 | 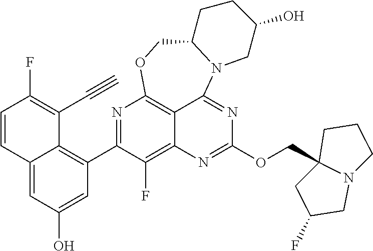 [SMILES: C#Cc1c(F)ccc2cc(O)cc(-c3nc4c5c(nc(OC[C@@]67CCCN6C[C@H](F)C7)nc5c3F)N3C[C@@H](O)CC[C@H]3CO4)c12] | 1H NMR (400 MHz, DMSO-d6 ) δ = 10.15 (d, J = 10.4 Hz, 1H), 8.03-7.90 (m, 1H), 7.49-7.41 (m, 1H), 7.37 (t, J = 2.8 Hz, 1H), 7.26- 7.11 (m, 1H), 5.38-5.00 (m, 2H), 4.79-4.66 (m, 1H), 4.51 ( s, 2H), 4.09-3.99 (m, 2H), 3.98-3.64 (m, 3H), 3.22 (br t, J = 13.2 Hz, 1H), 3.10 (d, J = 7.6 Hz, 2H), 3.02 ( s, 1H), 2.88-2.78 (m, 1H), 2.12 ( s, 2H), 2.06- 1.99 (m, 2H), 1.88-1.81 (m, 3H), 1.80-1.73 (m, 2H), 1.68-1.60 (m, 1H) |
| 87 | 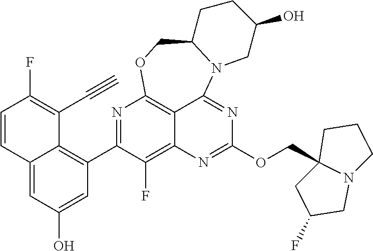 [SMILES: C#Cc1c(F)ccc2cc(O)cc(-c3nc4c5c(nc(OC[C@@]67CCCN6C[C@H](F)C7)nc5c3F)N3C[C@H](O)CC[C@@H]3CO4)c12] | 1H NMR (400 MHz, DMSO-d6 ) δ = 10.26- 10.03 (m, 1H), 7.99-7.93 (m, 1H), 7.49-7.42 (m, 1H), 7.37 (t, J = 2.8 Hz, 1H), 7.25- 7.12 (m, 1H), 5.36-5.04 (m, 2H), 4.78-4.67 (m, 1H), 4.58-4.46 (m, 2H), 4.12- 4.08 (m, 1H), 3.98-3.92 (m, 2H), 3.86 (d, J = 12.4 Hz, 1H), 3.82-3.65 (m, 1H), 3.22 ( t, J = 13.6 Hz, 1H), 3.09 ( d, J = 11.6 Hz, 2H), 3.02 (s, 1H), 2.87-2.78 (m, 1H), 2.13 ( d, J = 4.4 Hz, 1H), 2.09-1.95 (m, 3H), 1.88-1.74 (m, 5H), 1.64 ( d, J = 12.0 Hz, 1H) |
| 89 | 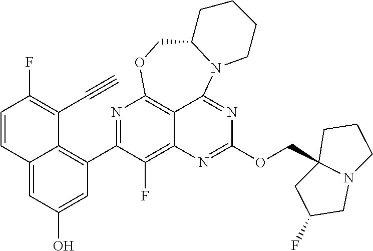 [SMILES: C#Cc1c(F)ccc2cc(O)cc(-c3nc4c5c(nc(OC[C@@]67CCCN6C[C@H](F)C7)nc5c3F)N3CCCC[C@H]3CO4)c12] | 1H NMR (400 MHz, DMSO-d6 ) δ = 8.23-8.15 (m, 1H), 8.04 (dd, J = 2.8, 9.4 Hz, 1H), 7.66 (s, 2H), 5.36-5.19 (m, 1H), 4.49 (br d, J = 13.6 Hz, 2H), 4.26- 4.18 (m, 1H), 4.11 (s, 1H), 4.01-3.91 (m, 2H), 3.13- 3.07 (m, 2H), 3.06-2.98 (m, 2H), 2.85-2.79 (m, 1H), 2.14-2.05 (m, 2H), 2.00- 1.68 (m, 11H) |
| 90 | 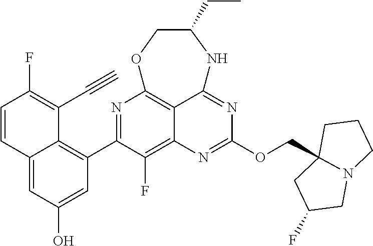 [SMILES: C#Cc1c(F)ccc2cc(O)cc(-c3nc4c5c(nc(OC[C@@]67CCCN6C[C@H](F)C7)nc5c3F)N[C@@H](CC)CO4)c12] | 1H NMR (400 MHz, DMSO-d6 ) δ = 10.56-9.82 (m, 1H), 9.32-9.22 (m, 1H), 7.95 (dd, J = 6.0, 9.2 Hz, 1H), 7.45 (t, J = 8.8 Hz, 1H), 7.36 (d, J = 2.4 Hz, 1H), 7.15 (dd, J = 2.4, 9.2 Hz, 1H), 5.38-5.17 (m, 1H), 4.54-4.35 (m, 2H), 4.12- 3.94 (m, 3H), 3.65 (br d, J = 0.8 Hz, 1H), 3.09 (br d, J = 10.4 Hz, 2H), 3.05-2.98 (m, 1H), 2.88-2.77 (m, 1H), 2.14-1.93 (m, 3H), 1.88- 1.74 (m, 3H), 1.70 (br s, 2H), 0.99 (t, J = 7.2 Hz, 3H) |
| 91 | 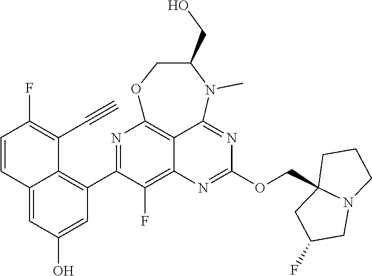 [SMILES: C#Cc1c(F)ccc2cc(O)cc(-c3nc4c5c(nc(OC[C@@]67CCCN6C[C@H](F)C7)nc5c3F)N(C)[C@H](CO)CO4)c12] | 1H NMR (400 MHz, METHANOL-d4 ) δ = 7.88- 7.81 (m, 1H), 7.37-7.18 (m, 3H), 5.51-5.31 (m, 1H), 4.54-4.37 (m, 3H), 4.16- 4.05 (m, 1H), 3.97-3.89 (m, 1H), 3.87-3.79 (m, 1H), 3.61 (br d, J = 3.2 Hz, 1H), 3.56-3.44 (m, 6H), 3.42 (s, 1H), 3.26-3.16 (m, 1H), 2.57-2.34 (m, 2H), 2.33- 2.21 (m, 1H), 2.19-2.10 (m, 2H), 2.03-1.95 (m, 1H) |
| 92 | 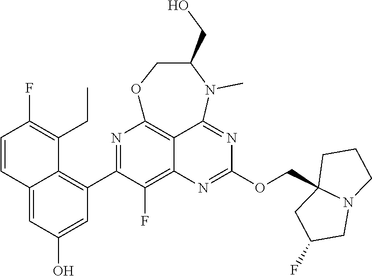 [SMILES: CCc1c(F)ccc2cc(O)cc(-c3nc4c5c(nc(OC[C@@]67CCCN6C[C@H](F)C7)nc5c3F)N(C)[C@H](CO)CO4)c12] | 1H NMR (400 MHz, METHANOL-d4 ) δ = 7.69- 7.62 (m, 1H), 7.31-7.19 (m, 2H), 7.14-6.98 (m, 1H), 5.46-5.26 (m, 1H), 4.52- 4.41 (m, 2H), 4.38-4.31 (m, 1H), 4.13-4.07 (m, 1H), 3.96-3.87 (m, 1H), 3.85- 3.77 (m, 1H), 3.58-3.34 (m, 6H), 3.17-3.09 (m, 1H), 2.64-2.29 (m, 4H), 2.28- 2.16 (m, 2H), 2.14-2.02 (m, 2H), 2.02-1.90 (m, 1H), 0.93-0.80 (m, 3H) |
| 93 | 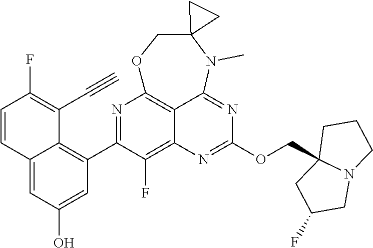 [SMILES: C#Cc1c(F)ccc2cc(O)cc(-c3nc4c5c(nc(OC[C@@]67CCCN6C[C@H](F)C7)nc5c3F)N(C)C3(CC3)CO4)c12] | 1H NMR (400 MHz, METHANOL-d4 ) δ = 7.84 (dd, J = 5.6, 9.1 Hz, 1H), 7.36-7.29 (m, 2H), 7.24 (d, J = 2.4 Hz, 1H), 5.51-5.29 (m, 1H), 4.78-4.71 (m, 1H), 4.51-4.44 (m, 1H), 4.43- 4.36 (m, 2H), 3.68-3.55 (m, 1H), 3.50 (br s, 3H), 3.42 (s, 3H), 3.24-3.13 (m, 1H), 2.52-2.30 (m, 2H), 2.23 (br d, J = 9.6 Hz, 1H), 2.18- 2.07 (m, 2H), 1.97 (br d, J = 5.6 Hz, 1H), 1.50-1.40 (m, 1H), 1.38-1.23 (m, 2H), 1.18 (br d, J = 4.8 Hz, 1H) |
| 94 | 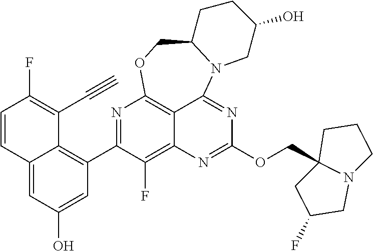 [SMILES: C#Cc1c(F)ccc2cc(O)cc(-c3nc4c5c(nc(OC[C@@]67CCCN6C[C@H](F)C7)nc5c3F)N3C[C@@H](O)CC[C@@H]3CO4)c12] | 1H NMR (400 MHz, DMSO-d6 ) δ = 10.13 (s, 1H), 8.00-7.91 (m, 1H), 7.46 (t, J = 9.1 Hz, 1H), 7.37 (d, J = 2.1 Hz, 1H), 7.14 (br d, J = 13.1 Hz, 1H), 5.45- 4.96 (m, 3H), 4.57-4.37 (m, 2H), 4.18-3.98 (m, 3H), 3.93-3.84 (m, 1H), 3.64- 3.45 (m, 1H), 3.13 (br d, J = 2.6 Hz, 2H), 3.08-3.00 (m, 1H), 2.92-2.82 (m, 1H), 2.76 (br t, J = 11.3 Hz, 1H), 2.15-2.07 (m, 2H), 2.06- 1.97 (m, 2H), 1.86 (br d, J = 12.6 Hz, 2H), 1.83-1.76 (m, 2H), 1.75-1.65 (m, 1H), 1.57 (br d, J = 9.6 Hz, 1H) |
| 95 | 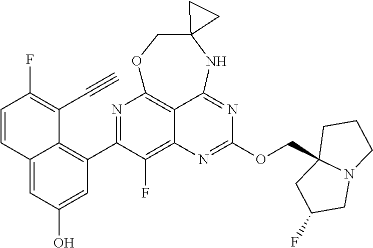 [SMILES: C#Cc1c(F)ccc2cc(O)cc(-c3nc4c5c(nc(OC[C@@]67CCCN6C[C@H](F)C7)nc5c3F)NC3(CC3)CO4)c12] | 1H NMR (400 MHz, DMSO-d6 ) δ = 10.41-9.89 (m, 1H), 9.45-9.34 (m, 1H), 8.01-7.89 (m, 1H), 7.51- 7.42 (m, 1H), 7.41-7.31 (m, 1H), 7.22-7.11 (m, 1H), 5.37-5.16 (m, 1H), 4.45- 4.30 (m, 2H), 4.12-3.96 (m, 3H), 3.10-2.99 (m, 3H), 2.89-2.76 (m, 1H), 2.12- 1.92 (m, 3H), 1.87-1.72 (m, 3H), 1.17-1.00 (m, 4H) |
| 96 | 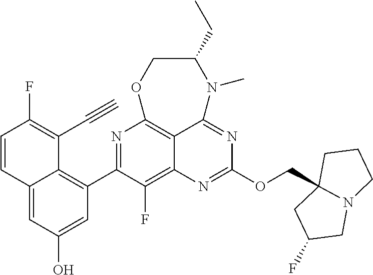 [SMILES: C#Cc1c(F)ccc2cc(O)cc(-c3nc4c5c(nc(OC[C@@]67CCCN6C[C@H](F)C7)nc5c3F)N(C)[C@@H](CC)CO4)c12] | 1H NMR (400 MHz, DMSO-d6 ) δ = 10.18- 10.09 (m, 1H), 7.96 (dd, J = 6.0, 9.2 Hz, 1H), 7.46 (dt, J = 2.8, 9.2 Hz, 1H), 7.37 (d, J = 2.4 Hz, 1H), 7.23-7.07 (m, 1H), 5.43-5.17 (m, 1H), 4.74-4.55 (m, 1H), 4.48- 4.32 (m, 1H), 4.21-3.83 (m, 4H), 3.38 (s, 3H), 3.18-2.97 (m, 3H), 2.89-2.77 (m, 1H), 2.14 (br s, 1H), 2.11-1.95 (m, 2H), 1.88-1.66 (m, 5H), 1.08-0.92 (m, 3H) |
| 97 | 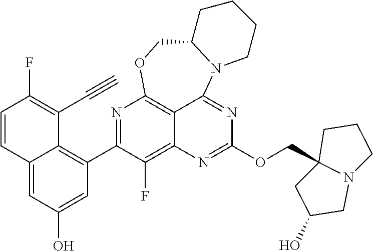 [SMILES: C#Cc1c(F)ccc2cc(O)cc(-c3nc4c5c(nc(OC[C@@]67CCCN6C[C@H](O)C7)nc5c3F)N3CCCC[C@H]3CO4)c12] | 1H NMR (400 MHz, METHANOL-d4 ) δ = 7.89- 7.82 (m, 1H), 7.36-7.28 (m, 2H), 7.26-7.18 (m, 1H), 5.40 (br d, J = 12.4 Hz, 1H), 4.67-4.46 (m, 6H), 4.01 (br d, J = 9.2 Hz, 1H), 3.68 (br s, 2H), 3.62-3.48 (m, 2H), 3.18-3.07 (m, 1H), 2.53- 2.43 (m, 1H), 2.42-2.24 (m, 3H), 2.15 (br d, J = 14.0 Hz, 1H), 2.11-2.00 (m, 2H), 1.96 (br d, J = 8.0 Hz, 1H), 1.92-1.77 (m, 3H), 1.72- 1.58 (m, 1H) |
| 98 | 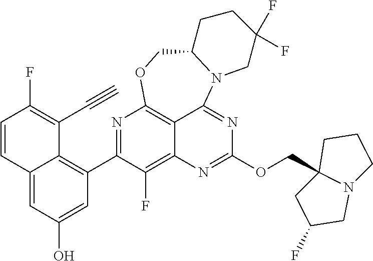 [SMILES: C#Cc1c(F)ccc2cc(O)cc(-c3nc4c5c(nc(OC[C@@]67CCCN6C[C@H](F)C7)nc5c3F)N3CC(F)(F)CC[C@H]3CO4)c12] | 1H NMR (400 MHz, DMSO-d6 ) δ = 10.16 (s, 1H), 8.00-7.93 (m, 1H), 7.46 (t, J = 9.2 Hz, 1H), 7.39- 7.35 (m, 1H), 7.20-7.13 (m, 1H), 5.47-5.37 (m, 1H), 5.36-5.21 (m, 1H), 4.67- 4.55 (m, 2H), 4.18-4.13 (m, 1H), 4.11-4.08 (m, 1H), 4.05-4.00 (m, 1H), 3.70- 3.52 (m, 1H), 3.11 (d, J = 8.4 Hz, 2H), 3.03 ( s, 1H), 2.89- 2.79 (m, 1H), 2.37-2.27 (m, 2H), 2.15 ( s, 1H), 2.10- 1.94 (m, 4H), 1.89-1.74 (m, 4H) |
| 99 | 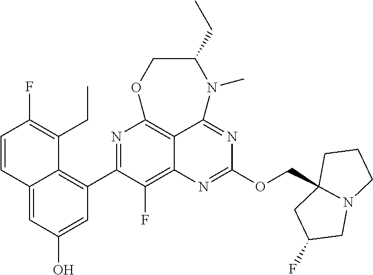 [SMILES: CCc1c(F)ccc2cc(O)cc(-c3nc4c5c(nc(OC[C@@]67CCCN6C[C@H](F)C7)nc5c3F)N(C)[C@@H](CC)CO4)c12] | 1H NMR (400 MHz, DMSO-d6 ) δ = 10.01-9.81 (m, 1H), 8.17 (s, 1H), 7.75 (dd, J = 6.4, 8.8 Hz, 1H), 7.38-7.29 (m, 2H), 7.10- 6.95 (m, 1H), 5.40-5.17 (m, 1H), 4.72-4.63 (m, 1H), 4.45 (br d, J = 12.4 Hz, 1H), 4.22-4.13 (m, 1H), 4.03 (br d, J = 10.8 Hz, 1H), 3.97- 3.90 (m, 1H), 3.39 (d, J = 2.4 Hz, 3H), 3.14-3.01 (m, 3H), 2.89-2.79 (m, 1H), 2.27- 2.11 (m, 2H), 2.10-1.97 (m, 2H), 1.93-1.54 (m, 5H), 0.99 (q, J = 6.8 Hz, 3H), 0.88-0.73 (m, 3H) |
| 100 | 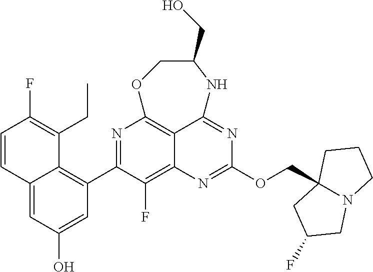 [SMILES: CCc1c(F)ccc2cc(O)cc(-c3nc4c5c(nc(OC[C@@]67CCCN6C[C@H](F)C7)nc5c3F)N[C@H](CO)CO4)c12] | 1H NMR (400 MHz, METHANOLd4 ) δ = 7.71- 7.60 (m, 1H), 7.32-7.18 (m, 2H), 7.10-7.03 (m, 1H), 5.40-5.19 (m, 1H), 4.63- 4.51 (m, 2H), 4.32-4.18 (m, 2H), 3.97-3.87 (m, 1H), 3.86-3.70 (m, 2H), 3.07- 2.96 (m, 1H), 2.64-2.42 (m, 1H), 2.40-2.16 (m, 3H), 2.15-2.06 (m, 1H), 2.05- 1.82 (m, 3H), 1.31 (br d, J = 16.8 Hz, 3H), 0.95-0.80 (m, 3H) |
| 101 | 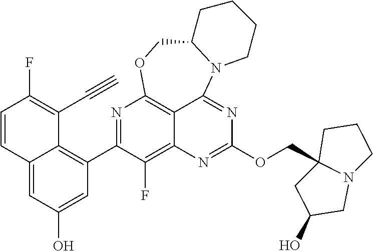 [SMILES: C#Cc1c(F)ccc2cc(O)cc(-c3nc4c5c(nc(OC[C@@]67CCCN6C[C@@H](O)C7)nc5c3F)N3CCCC[C@H]3CO4)c12] | 1H NMR (400 MHz, METHANOL-d4 ) δ = 7.85 (dd, J = 6.0, 9.2 Hz, 1H), 7.36-7.29 (m, 2H), 7.21 (dd, J = 2.0, 19.6 Hz, 1H), 5.40 (br d, J = 13.6 Hz, 1H), 4.97 (br d, J = 12.0 Hz, 1H), 4.71-4.61 (m, 1H), 4.58 (br s, 1H), 4.55 (br s, 2H), 4.00 (br d, J = 8.8 Hz, 1H), 3.64- 3.54 (m, 2H), 3.52 (d, J = 5.3 Hz, 1H), 3.25 (br dd, J = 2.4, 12.4 Hz, 1H), 3.19-3.08 (m, 1H), 2.47-2.35 (m, 1H), 2.34-2.24 (m, 2H), 2.21 (br dd, J = 4.0, 10.8 Hz, 2H), 2.04 (br d, J = 12.4 Hz, 2H), 1.96 (br d, J = 4.4 Hz, 1H), 1.92-1.74 (m, 4H), 1.66 (br d, J = 12.8 Hz, 1H) |
| 102 | 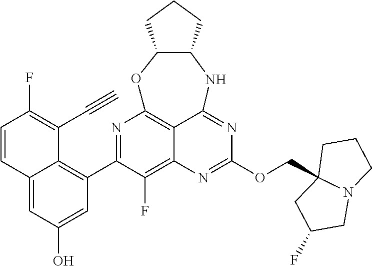 [SMILES: C#Cc1c(F)ccc2cc(O)cc(-c3nc4c5c(nc(OC[C@@]67CCCN6C[C@H](F)C7)nc5c3F)N[C@H]3CCC[C@H]3O4)c12] | 1H NMR (400 MHz, DMSO-d6 ) δ ppm 1.67- 1.90 (m, 3 H) 1.93-2.24 (m, 7 H) 2.25-2.35 (m, 1 H) 2.46 (br s, 1 H) 3.66-3.82 (m, 3 H) 3.85-3.94 (m, 2 H) 4.17 (d, J = 0.82 Hz, 1 H) 4.49-4.64 (m, 2 H) 4.72 (br s, 1 H) 5.49-5.64 (m, 1 H) 7.09-7.21 (m, 1 H) 7.38 (d, J = 2.46 Hz, 1 H) 7.47 (td, J = 8.97, 3.15 Hz, 1 H) 7.97 (ddd, J = 9.24, 5.82, 3.29 Hz, 1 H) 9.33 (dd, J = 12.59, 5.20 Hz, 1 H) 10.18 (br s, 1 H) 10.76-10.98 (m, 1 H) |
| 103 | 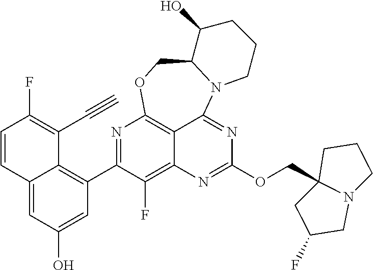 [SMILES: C#Cc1c(F)ccc2cc(O)cc(-c3nc4c5c(nc(OC[C@@]67CCCN6C[C@H](F)C7)nc5c3F)N3CCC[C@H](O)[C@@H]3CO4)c12] | 1H NMR (400 MHz, METHANOL-d4 ) δ = 7.86 (br dd, J = 6.0, 8.9 Hz, 1H), 7.42-7.28 (m, 2H), 7.25- 7.15 (m, 1H), 5.58-5.36 (m, 1H), 5.35-5.19 (m, 1H), 4.69-4.41 (m, 5H), 4.30- 4.05 (m, 2H), 3.83-3.45 (m, 4H), 3.29-3.11 (m, 2H), 2.59-1.94 (m, 9H), 1.80- 1.58 (m, 1H) |
| 104 | 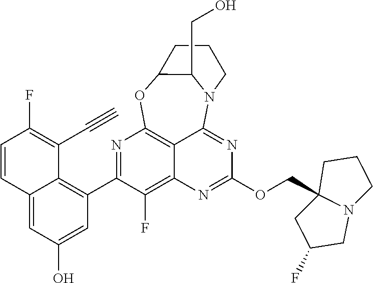 [SMILES: C#Cc1c(F)ccc2cc(O)cc(-c3nc4c5c(nc(OC[C@@]67CCCN6C[C@H](F)C7)nc5c3F)N3CCCC(O4)C3CO)c12] | 1H NMR (400 MHz, METHANOL-d4 ) δ = 7.89- 7.80 (m, 1H), 7.39-7.27 (m, 2H), 7.25-7.16 (m, 1H), 5.28 (br s, 1H), 4.64-4.61 (m, 3H), 4.44-4.22 (m, 2H), 4.21-4.03 (m, 2H), 3.51- 3.46 (m, 1H), 3.28-2.99 (m, 3H), 2.45-2.13 (m, 4H), 1.90 (br s, 1H), 1.77-1.62 (m, 1H) |
| 105 | 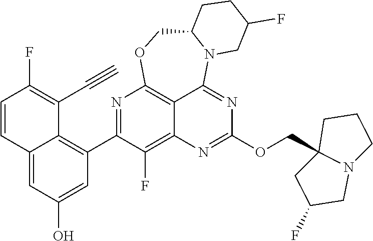 [SMILES: C#Cc1c(F)ccc2cc(O)cc(-c3nc4c5c(nc(OC[C@@]67CCCN6C[C@H](F)C7)nc5c3F)N3CC(F)CC[C@H]3CO4)c12] | 1H NMR (400 MHz, DMSO-d6 ) δ = 10.18- 10.10 (m, 1H), 7.99-7.91 (m, 1H), 7.49-7.43 (m, 1H), 7.37 (d, J = 2.4 Hz, 1H), 7.21-7.08 (m, 1H), 5.36- 5.10 (m, 2H), 4.59-4.42 (m, 2H), 4.17-3.97 (m, 4H), 3.10 (d, J = 9.6 Hz, 2H), 3.03 ( s, 1H), 2.88-2.80 (m, 1H), 2.37-2.23 (m, 2H), 2.15-2.06 (m, 2H), 2.02- 1.93 (m, 2H), 1.88-1.82 (m, 2H), 1.81-1.76 (m, 2H), 1.47 (s, 2H), 1.40 (s, 2H), 1.23 (s, 2H) |
| 106 | 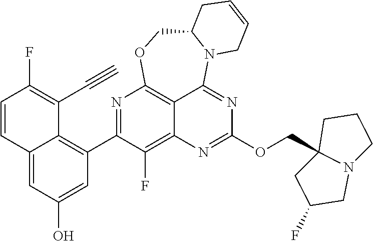 [SMILES: C#Cc1c(F)ccc2cc(O)cc(-c3nc4c5c(nc(OC[C@@]67CCCN6C[C@H](F)C7)nc5c3F)N3CC=CC[C@H]3CO4)c12] | 1H NMR (400 MHz, DMSO-d6 ) δ = 10.22- 10.07 (m, 1H), 8.00-7.92 (m, 1H), 7.50-7.42 (m, 1H), 7.37 (d, J = 2.0 Hz, 1H), 7.21-7.09 (m, 1H), 5.98- 5.78 (m, 2H), 5.38-5.19 (m, 1H), 5.09-4.97 (m, 1H), 4.64-4.51 (m, 2H), 4.13- 4.11 (m, 1H), 4.02-3.99 (m, 1H), 3.10 (d, J = 9.2 Hz, 2H), 3.02 (s, 1H), 2.87-2.79 (m, 1H), 2.34-2.27 (m, 1H), 2.14-2.04 (m, 2H), 2.02- 1.97 (m, 1H), 1.86-1.81 (m, 1H), 1.80-1.70 (m, 2H), 1.47 (s, 2H), 1.23 (s, 2H) |
| 107 | 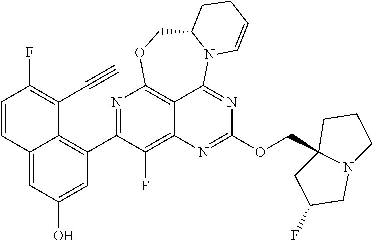 [SMILES: C#Cc1c(F)ccc2cc(O)cc(-c3nc4c5c(nc(OC[C@@]67CCCN6C[C@H](F)C7)nc5c3F)N3C=CCC[C@H]3CO4)c12] | 1H NMR (400 MHz, DMSO-d6 ) δ = 10.28-9.96 (m, 1H), 7.99-7.94 (m, 1H), 7.49-7.43 (m, 1H), 7.37 (s, 1H), 7.22-7.10 (m, 1H), 5.44 (d, J= 3.2 Hz, 1H), 5.37- 5.18 (m, 1H), 4.69-4.47 (m, 2H), 4.16-3.99 (m, 4H), 3.09 (d, J = 8.4 Hz, 2H), 3.02 (s, 1H), 2.86-2.80 (m, 1H), 2.29-2.20 (m, 1H), 2.13 (d, J = 1.2 Hz, 1H), 2.03 (d, J = 15.2 Hz, 2H), 1.86-1.80 (m, 2H), 1.79- 1.76 (m, 1H), 1.47 (s, 2H), 1.25-1.22 (m, 2H) |
| 108 | 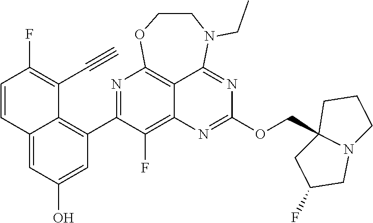 [SMILES: C#Cc1c(F)ccc2cc(O)cc(-c3nc4c5c(nc(OC[C@@]67CCCN6C[C@H](F)C7)nc5c3F)N(CC)CCO4)c12] | 1H NMR (400 MHz, DMSO-d6 ) δ = 10.39-9.94 (m, 1H), 7.95 (dd, J = 5.6, 9.2 Hz, 1H), 7.45 (t, J = 9.2 Hz, 1H), 7.36 (d, J = 2.4 Hz, 1H), 7.13 (d, J = 2.4 Hz, 1H), 5.39-5.15 (m, 1H), 4.52 (s, 2H), 4.15-4.07 (m, 2H), 4.02 (dd, J = 7.6, 10.4 H7, 1H), 3.98-3.91 (m, 2H), 3.78 (qd, J = 6.4, 13.2 Hz, 1H), 3.14-3.04 (m, 2H), 3.01 (s, 1H), 2.88-2.76 (m, 2H), 2.13 (s, 1H), 2.05 (s, 1H), 2.02-1.96 (m, 1H), 1.88-1.73 (m, 3H), 1.25 (t, J = 7.2 Hz, 3H) |
| 109 | 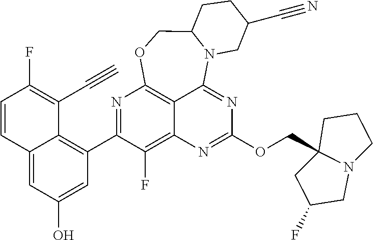 [SMILES: C#Cc1c(F)ccc2cc(O)cc(-c3nc4c5c(nc(OC[C@@]67CCCN6C[C@H](F)C7)nc5c3F)N3CC(C#N)CCC3CO4)c12] | 1H NMR (400 MHz, METHANOL-d4 ) δ = 7.92- 7.80 (m, 1H), 7.40-7.29 (m, 2H), 7.25-7.12 (m, 1H), 5.64-5.38 (m, 1H), 5.32- 5.19 (m, 1H), 4.59-4.48 (m, 3H), 4.41-4.22 (m, 3H), 4.12-3.96 (m, 2H), 3.54- 3.51 (m, 1H), 3.11-3.03 (m, 2H), 2.49-2.04 (m, 10H) |
| 110 | 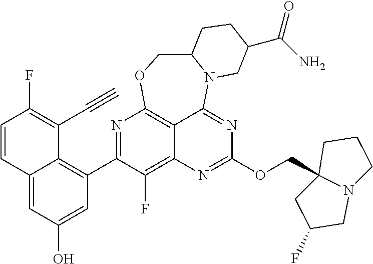 [SMILES: C#Cc1c(F)ccc2cc(O)cc(-c3nc4c5c(nc(OC[C@@]67CCCN6C[C@H](F)C7)nc5c3F)N3CC(C(N)=O)CCC3CO4)c12] | 1H NMR (400 MHz, DMSO-d6 ) δ = 10.21- 10.09 (m, 1H), 7.90 (s, 2H), 7.53-7.40 (m, 1H), 7.34- 7.24 (m, 1H), 5.40-5.19 (m, 1H), 4.83-4.42 (m, 2H), 4.25-3.99 (m, 2H), 3.19- 2.98 (m, 6H), 2.91-2.79 (m, 1H), 2.54-2.53 (m, 2H), 2.29-1.97 (m, 6H), 1.91- 1.67 (m, 6H) |
| 111 | 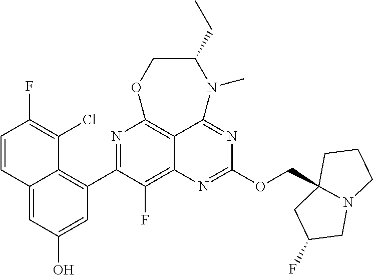 [SMILES: CC[C@H]1COc2nc(-c3cc(O)cc4ccc(F)c(Cl)c34)c(F)c3nc(OC[C@@]45CCCN4C[C@H](F)C5)nc(c23)N1C] | 1H NMR (400 MHz, DMSO-d6 ) δ = 8.29 (s, 1H), 7.98-7.88 (m, 1H), 7.56 (dt, J = 5.2, 8.8 Hz, 1H), 7.45- 7.37 (m, 1H), 7.29-7.11 (m, 1H), 5.39-5.17 (m, 1H), 4.72-4.60 (m, 1H), 4.49- 4.37 (m, 1H), 4.17-4.10 (m, 1H), 4.06-3.98 (m, 1H), 3.97-3.88 (m, 1H), 3.37 (s, 3H), 3.13-3.06 (m, 2H), 3.01 (s, 1H), 2.87-2.79 (m, 1H), 2.15-2.11 (m, 1H), 2.07-1.97 (m, 2H), 1.88- 1.61 (m, 5H), 1.05-0.94 (m, 3H) |
| 112 | 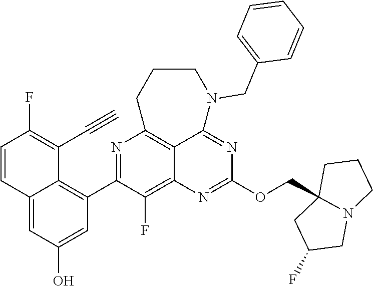 [SMILES: C#Cc1c(F)ccc2cc(O)cc(-c3nc4c5c(nc(OC[C@@]67CCCN6C[C@H](F)C7)nc5c3F)N(Cc3ccccc3)CCC4)c12] | 1H NMR (400 MHz, DMSO-d6 ) δ = 7.99-7.92 (m, 1H), 7.50-7.37 (m, 6H), 7.35-7.30 (m, 1H), 7.19 (d, J = 2.4 Hz, 1H), 5.34-5.14 (m, 2H), 4.80 (d, J = 14.8 Hz, 1H), 4.12 (s, 1H), 4.11- 3.95 (m, 2H), 3.60-3.45 (m, 3H), 3.11-3.04 (m, 3H), 3.00-2.91 (m, 2H), 2.84- 2.77 (m, 1H), 2.11-2.05 (m, 3H), 2.02-1.95 (m, 2H), 1.85-1.71 (m, 3H) |
| 113 | 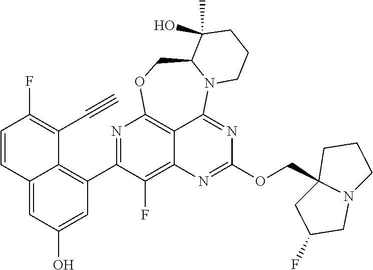 [SMILES: C#Cc1c(F)ccc2cc(O)cc(-c3nc4c5c(nc(OC[C@@]67CCCN6C[C@H](F)C7)nc5c3F)N3CCC[C@](C)(O)[C@@H]3CO4)c12] | 1H NMR (400 MHz, DMSO-d6 ) δ = 10.15- 10.10 (m, 1H), 7.99-7.91 (m, 1H), 7.45 (t, J = 9.1 Hz, 1H), 7.36 (d, J = 2.0 Hz, 1H), 7.17-7.07 (m, 1H), 5.40-5.20 (m, 1H), 5.19- 5.04 (m, 1H), 4.75-4.59 (m, 1H), 4.59-4.46 (m, 1H), 4.44-4.36 (m, 1H), 4.20- 3.93 (m, 3H), 3.93-3.81 (m, 1H), 3.18-3.06 (m, 2H), 3.06-2.98 (m, 2H), 2.89- 2.79 (m, 1H), 2.17-2.06 (m, 2H), 2.05-1.94 (m, 2H), 1.91-1.84 (m, 1H), 1.83- 1.75 (m, 2H), 1.73-1.64 (m, 2H), 1.60-1.54 (m, 1H), 1.28 (d, J = 2.8 Hz, 3H) |
| 114 | 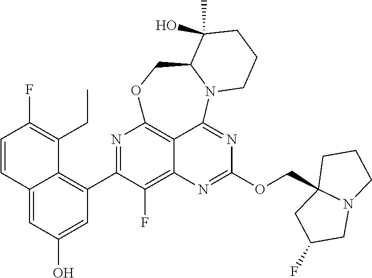 [SMILES: CCc1c(F)ccc2cc(O)cc(-c3nc4c5c(nc(OC[C@@]67CCCN6C[C@H](F)C7)nc5c3F)N3CCC[C@](C)(O)[C@@H]3CO4)c12] | 1H NMR (400 MHz, DMSO-d6 ) δ = 9.90 ( s, 1H), 7.78-7.71 (m, 1H), 7.33 ( t, J = 9.6 Hz, 1H), 7.30 ( s, 1H), 7.02-6.95 (m, 1H), 5.38-5.19 (m, 1H), 5.10 (d, J = 12.1 H7, 1H), 4.76-4.61 (m, 1H), 4.56- 4.49 (m, 1H), 4.45 ( d, J = 18.8 Hz, 1H), 4.15-4.03 (m, 2H), 3.87 ( d, J = 2.8 Hz, 1H), 3.10 (d, J = 7.6 Hz, 2H), 3.06-2.98 (m, 2H), 2.88-2.78 (m, 1H), 2.44- 2.35 (m, 1H), 2.34-2.23 (m, 1H), 2.17-2.12 (m, 1H), 2.06 ( s, 1H), 2.04-1.99 (m, 1H), 1.99-1.93 (m, 1H), 1.84 ( d, J = 16.0 Hz, 1H), 1.81-1.75 (m, 2H), 1.73- 1.63 (m, 2H), 1.57 (d, J = 12.0 Hz, 1H), 1.27 ( d, J = 4.8 Hz, 3H), 0.82-0.75 (m, 3H) |
| 115 | 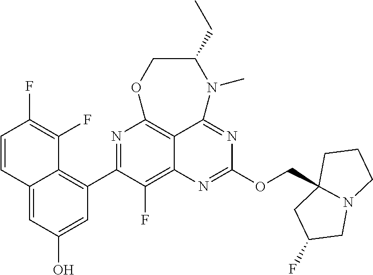 [SMILES: CC[C@H]1COc2nc(-c3cc(O)cc4ccc(F)c(F)c34)c(F)c3nc(OC[C@@]45CCCN4C[C@H](F)C5)nc(c23)N1C] | 1H NMR (400 MHz, DMSO-d6 ) δ = 7.76-7.69 (m, 1H), 7.61-7.52 (m, 1H), 7.36 (s, 1H), 7.29-7.14 (m, 1H), 5.39-5.19 (m, 1H), 4.66 (dd, J = 4.4, 13.2 Hz, 1H), 4.44 (dd, J = 4.0, 13.6 Hz, 1H), 4.19-4.11 (m, 1H), 4.08-3.99 (m, 1H), 3.92 (d, J = 4.0 Hz, 1H), 3.37 (s, 3H), 3.09 (d, J = 8.4 Hz, 2H), 3.01 (s, 1H), 2.87-2.77 (m, 1H), 2.16-2.09 (m, 1H), 2.05 (s, 1H), 2.03-1.96 (m, 1H), 1.89-1.57 (m, 5H), 1.06-0.95 (m, 3H) |
| 116 | 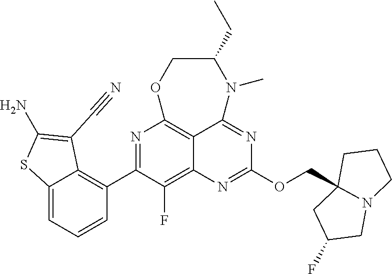 [SMILES: CC[C@H]1COc2nc(-c3cccc4sc(N)c(C#N)c34)c(F)c3nc(OC[C@@]45CCCN4C[C@H](F)C5)nc(c23)N1C] | 1H NMR (400 MHz, DMSO-d6 ) δ = 7.84-7.70 (m, 3H), 7.32 (d, J = 7.6 Hz, 1H), 7.25-7.16 (m, 1H), 5.38-5.17 (m, 1H), 4.71- 4.60 (m, 1H), 4.37 (d, J = 13.2 Hz, 1H), 4.18-3.98 (m, 2H), 3.95-3.83 (m, 1H), 3.36 (s, 3H), 3.09 (d, J = 7.6 Hz, 2H), 3.02 (s, 1H), 2.87- 2.77 (m, 1H), 2.13 (d, J = 6.4 Hz, 1H), 2.07-1.99 (m, 2H), 1.86-1.64 (m, 5H), 1.03- 0.94 (m, 3H) |
| 117 | 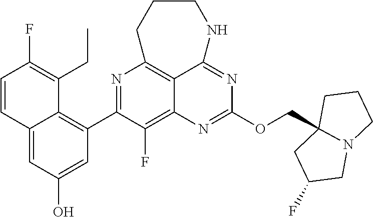 [SMILES: CCc1c(F)ccc2cc(O)cc(-c3nc4c5c(nc(OC[C@@]67CCCN6C[C@H](F)C7)nc5c3F)NCCC4)c12] | 1H NMR (400 MHz, DMSO-d6 ) δ = 9.07-8.71 (m, 1H), 8.39 (s, 1H), 7.80- 7.66 (m, 1H), 7.39-7.28 (m, 2H), 7.00 (d, J = 2.4 Hz, 1H), 5.40-5.14 (m, 1H), 4.13-3.98 (m, 2H), 3.25- 3.20 (m, 3H), 3.11-3.06 (m, 2H), 3.01 (s, 1H), 2.87-2.77 (m, 1H), 2.30-2.18 (m, 2H), 2.13-2.08 (m, 2H), 2.04- 1.96 (m, 2H), 1.87-1.73 (m, 3H), 1.28-1.19 (m, 2H), 0.81-0.73 (m, 3H) |
| 118 | 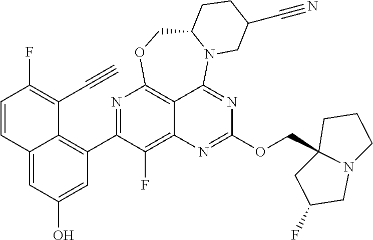 [SMILES: C#Cc1c(F)ccc2cc(O)cc(-c3nc4c5c(nc(OC[C@@]67CCCN6C[C@H](F)C7)nc5c3F)N3CC(C#N)CC[C@H]3CO4)c12] | 1H NMR (400 MHz, METHANOL-d4 ) δ = 7.88- 7.79 (m, 1H), 7.36-7.28 (m, 2H), 7.24-7.16 (m, 1H), 5.42-5.32 (m, 1H), 4.41- 3.78 (m, 6H), 3.25-3.16 (m, 2H), 3.07-2.96 (m, 1H), 2.64-2.49 (m, 1H), 2.34- 2.14 (m, 2H), 2.00 (br d, J = 8.0 Hz, 4H), 1.34-1.30 (m, 4H), 0.94-0.86 (m, 3H) |
| 119 | 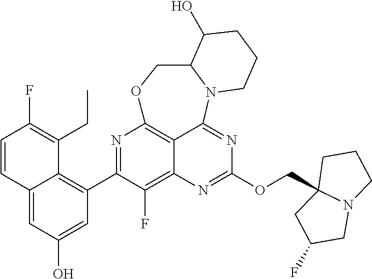 [SMILES: CCc1c(F)ccc2cc(O)cc(-c3nc4c5c(nc(OC[C@@]67CCCN6C[C@H](F)C7)nc5c3F)N3CCCC(O)C3CO4)c12] | 1H NMR (400 MHz, METHANOL-d4 ) δ = 7.65 (dd, J = 6.0, 9.0 Hz, 1H), 7.29-7.19 (m, 2H), 7.05 (dd, J = 2.4, 14.3 Hz, 1H), 5.41-5.16 (m, 2H), 4.70- 4.55 (m, 3H), 4.39-4.01 (m, 4H), 3.26-3.13 (m, 3H), 3.06-2.96 (m, 1H), 2.60- 2.44 (m, 1H), 2.35 (br t, J = 4.4 Hz, 1H), 2.30-2.20 (m, 2H), 2.19-2.07 (m, 2H), 2.06-1.89 (m, 5H), 1.73- 1.53 (m, 1H), 0.91-0.79 (m, 3H) |
| 120 | 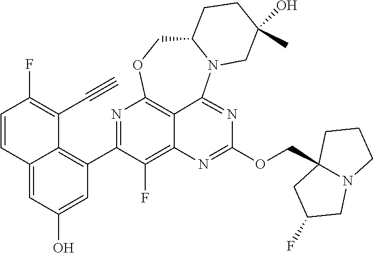 [SMILES: C#Cc1c(F)ccc2cc(O)cc(-c3nc4c5c(nc(OC[C@@]67CCCN6C[C@H](F)C7)nc5c3F)N3C[C@@](C)(O)CC[C@H]3CO4)c12] | 1H NMR (400 MHz, DMSO-d6 ) δ = 10.14 (br s, 1H), 7.99-7.90 (m, 1H), 7.46 (br d, J = 1.6 Hz, 1H), 7.36 (d, J = 2.4 Hz, 1H), 7.19-7.13 (m, 1H), 5.40- 5.15 (m, 1H), 5.03 (s, 1H), 4.50 (br s, 1H), 4.14-4.08 (m, 1H), 4.04-3.98 (m, 1H), 3.85 (s, 1H), 3.13-3.07 (m, 2H), 3.04-3.00 (m, 1H), 2.91 (br d, J = 12.4 Hz, 1H), 2.88-2.78 (m, 2H), 2.17- 2.10 (m, 2H), 2.05-1.96 (m, 4H), 1.87-1.81 (m, 2H), 1.78 (br d, J = 5.2 Hz, 4H), 1.09 (d, J = 18.8 Hz, 3H) |
| 121 | 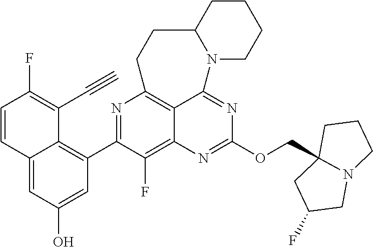 [SMILES: C#Cc1c(F)ccc2cc(O)cc(-c3nc4c5c(nc(OC[C@@]67CCCN6C[C@H](F)C7)nc5c3F)N3CCCCC3CC4)c12] | 1H NMR (400 MHz, METHANOL-d4 ) δ = 8.52 (s, 1H), 7.88-7.82 (m, 1H), 7.35-7.28 (m, 2H), 7.22- 7.16 (m, 1H), 5.46-5.29 (m, 1H), 4.78-4.63 (m, 1H), 4.47-4.31 (m, 2H), 3.94- 3.80 (m, 1H), 3.49-3.36 (m, 4H), 3.25-3.10 (m, 3H), 2.56-2.29 (m, 3H), 2.26- 2.17 (m, 1H), 2.13-2.05 (m, 2H), 2.01-1.84 (m, 6H), 1.80-1.69 (m, 1H), 1.58- 1.44 (m, 1H) |
| 122 | 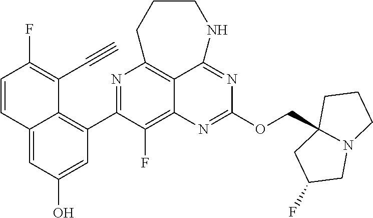 [SMILES: C#Cc1c(F)ccc2cc(O)cc(-c3nc4c5c(nc(OC[C@@]67CCCN6C[C@H](F)C7)nc5c3F)NCCC4)c12] | 1H NMR (400 MHz, DMSO-d6 ) δ = 10.17 (s, 1H), 9.17-8.88 (m, 1H), 8.05-7.89 (m, 1H), 7.49- 7.43 (m, 1H), 7.38 (d, J = 2.4 Hz, 1H), 7.23-7.16 (m, 2H), 6.68-6.61 (m, 1H), 5.69- 5.44 (m, 1H), 5.34-5.30 (m, 2H), 4.64-4.45 (m, 2H), 3.83-3.64 (m, 3H), 3.21 (s, 2H), 2.20-2.09 (m, 5H), 1.48-1.43 (m, 3H) |
| 123 | 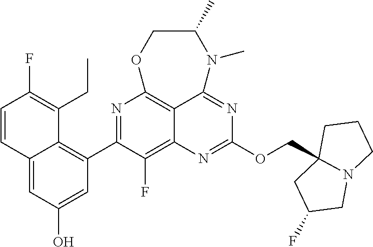 [SMILES: CCc1c(F)ccc2cc(O)cc(-c3nc4c5c(nc(OC[C@@]67CCCN6C[C@H](F)C7)nc5c3F)N(C)[C@@H](C)CO4)c12] | 1H NMR (400 MHz, DMSO-d6 ) δ = 10.07-9.83 (m, 1H), 7.79-7.70 (m, 1H), 7.40-7.27 (m, 2H), 7.10- 6.94 (m, 1H), 5.24 (s, 1H), 4.59 (s, 2H), 4.18-3.99 (m, 3H), 3.14-3.01 (m, 3H), 2.88-2.77 (m, 1H), 2.53- 2.51 (m, 2H), 2.35-2.16 (m, 2H), 2.15-2.02 (m, 2H), 1.87-1.72 (m, 3H), 1.31- 1.24 (m, 3H), 0.87-0.74 (m, 3H) |
| 124 | 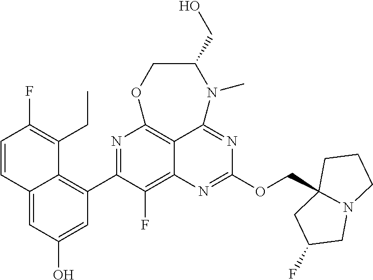 [SMILES: CCc1c(F)ccc2cc(O)cc(-c3nc4c5c(nc(OC[C@@]67CCCN6C[C@H](F)C7)nc5c3F)N(C)[C@@H](CO)CO4)c12] | 1H NMR (400 MHz, METHANOL-d4 ) δ = 7.70- 7.62 (m, 1H), 7.31-7.20 (m, 2H), 7.07 (br d, J = 11.2 Hz, 1H), 5.46-5.29 (m, 1H), 4.75-4.66 (m, 1H), 4.66- 4.53 (m, 2H), 4.43-4.31 (m, 2H), 3.98-3.89 (m, 1H), 3.87-3.72 (m, 2H), 3.56- 3.37 (m, 3H), 3.20-3.10 (m, 1H), 2.58-2.45 (m, 1H), 2.40-2.24 (m, 3H), 2.22- 2.14 (m, 1H), 2.14-1.90 (m, 3H), 0.91-0.83 (m, 3H) |
| 125 | 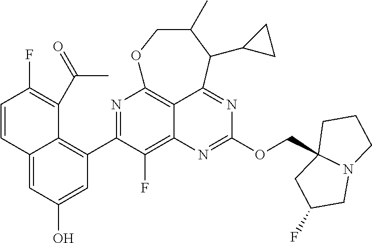 [SMILES: CC(=O)c1c(F)ccc2cc(O)cc(-c3nc4c5c(nc(OC[C@@]67CCCN6C[C@H](F)C7)nc5c3F)C(C3CC3)C(C)CO4)c12] | 1H NMR (400 MHz, METHANOL-d4 ) δ = 8.06 (s, 1H), 7.85-7.80 (m, 1H), 7.34-7.27 (m, 1H), 7.21 (d, J = 2.0 Hz, 1H), 5.41-5.23 (m, 1H), 4.74-4.66 (m, 1H), 4.59-4.53 (m, 1H), 4.38- 4.24 (m, 2H), 4.16-4.08 (m, 1H), 3.22 (br s, 1H), 3.06- 2.96 (m, 2H), 2.64-2.60 (m, 3H), 2.31-2.22 (m, 2H), 2.19-2.12 (m, 1H), 2.04- 1.98 (m, 2H), 1.96-1.87 (m, 1H), 1.37 (d, J = 6.4 Hz, 3H), 1.35-1.28 (m, 2H), 1.05-0.99 (m, 1H), 0.95- 0.89 (m, 1H), 0.81-0.75 (m, 1H), 0.56-0.48 (m, 1H) |
| 126 | 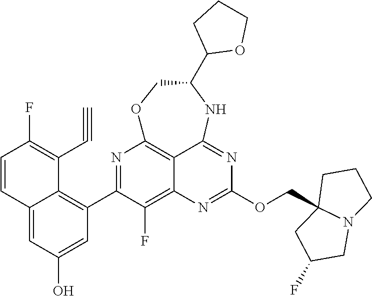 [SMILES: C#Cc1c(F)ccc2cc(O)cc(-c3nc4c5c(nc(OC[C@@]67CCCN6C[C@H](F)C7)nc5c3F)N[C@@H](C3CCCO3)CO4)c12] | 1H NMR (400 MHz, METHANOL-d4 ) δ = 7.88- 7.80 (m, 1H), 7.36-7.27- (m, 2H), 7.24-7.17 (m, 1H), 5.48-5.28 (m, 1H), 4.52 (t, J = 12.8 Hz, 1H), 4.46-4.31 (m, 2H), 4.22-4.11 (m, 1H), 3.97-3.89 (m, 1H), 3.88- 3.84 (m, 1H), 3.83-3.76 (m, 1H), 3.59-3.51 (m, 1H), 3.51-3.40 (m, 3H), 3.22- 3.12 (m, 1H), 2.50-2.26 (m, 2H), 2.24-1.80 (m, 9H) |
| 127 | 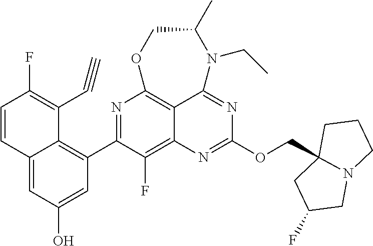 [SMILES: C#Cc1c(F)ccc2cc(O)cc(-c3nc4c5c(nc(OC[C@@]67CCCN6C[C@H](F)C7)nc5c3F)N(CC)[C@@H](C)CO4)c12] | ¹H NMR (400 MHz, DMSO- d₆) δ = 10.18-10.10 (m, 1H), 7.96 (dd, J = 5.6, 9.2 Hz, 1H), 7.50-7.42 (m, 1H), 7.40-7.34 (m, 1H), 7.22- 7.08 (m, 1H), 5.39-5.19 (m, 1H), 4.61-4.50 (m, 1H), 4.34 (d, J = 12.4 Hz, 1H), 4.20-4.08 (m, 3H), 4.08- 3.98 (m, 2H), 3.65-3.47 (m, 1H), 3.14-2.99 (m, 3H), 2.88-2.79 (m, 1H), 2.17- 1.97 (m, 3H), 1.91-1.73 (m, 3H), 1.34-1.25 (m, 6H). |
| 128 | 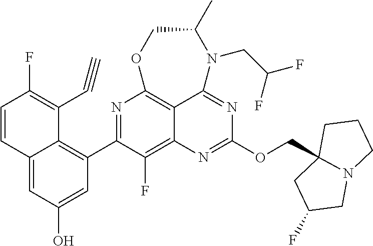 [SMILES: C#Cc1c(F)ccc2cc(O)cc(-c3nc4c5c(nc(OC[C@@]67CCCN6C[C@H](F)C7)nc5c3F)N(CC(F)F)[C@@H](C)CO4)c12] | ¹H NMR (400 MHz, MeOD- d₄) δ = 7.92-7.79 (m, 1H), 7.37-7.28 (m, 2H), 7.27- 7.14 (m, 1H), 6.61-6.25 (m, 1H),5.45-5.18 (m, 1H), 4.76-4.61 (m, 1H), 4.61- 4.43 (m, 2H), 4.35-4.20 (m, 3H), 4.15-3.98 (m, 1H), 3.54-3.41 (m, 1H), 3.30- 3.17 (m, 3H), 3.11-2.95 (m, 1H), 2.33 (d, J = 4.4 Hz, 2H), 2.16 (s, 1H), 2.06-1.96 (m, 2H), 1.95-1.83 (m, 1H), 1.42 (d, J = 6.8 Hz, 3H). |
| 129 | 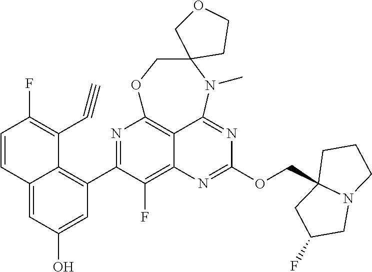 [SMILES: C#Cc1c(F)ccc2cc(O)cc(-c3nc4c5c(nc(OC[C@@]67CCCN6C[C@H](F)C7)nc5c3F)N(C)C3(CCOC3)CO4)c12] | ¹H NMR (400 MHz, MeOD- d₄) δ = 7.83 (dd, J = 5.6, 9.2 Hz, 1H), 7.34-7.28 (m, 2H), 7.20 (t, J = 2.8 Hz, 1H), 5.39- 5.22 (m, 1H), 4.57 (s, 2H), 4.35-4.23 (m, 2H), 4.21- 4.10 (m, 2H), 3.99-3.88 (m, 2H), 3.49 (dd, J = 2.8, 10.8 Hz, 1H), 3.41 (d, J = 1.4 Hz, 3H), 3.28-3.22 (m, 2H), 3.22-3.17 (m,1H), 3.02 (dt, J = 5.6, 9.2 Hz, 1H), 2.52- 2.36 (m, 1H), 2.36-2.19 (m, 3H), 2.18-2.08 (m, 1H), 2.05-1.95 (m, 2H), 1.94- 1.84 (m, 1H). |
| 130 | 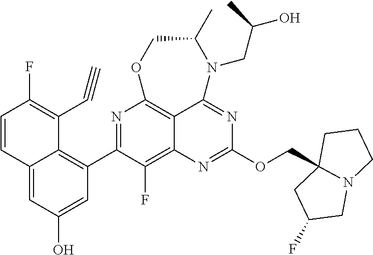 [SMILES: C#Cc1c(F)ccc2cc(O)cc(-c3nc4c5c(nc(OC[C@@]67CCCN6C[C@H](F)C7)nc5c3F)N(C[C@@H](C)O)[C@@H](C)CO4)c12] | ¹H NMR (400 MHz, MeOD- d₄) δ = 7.89-7.80 (m, 1H), 7.37-7.14 (m, 3H), 5.51- 5.32 (m, 1H), 4.69-4.61 (m, 1H),4.59-4.52 (m, 1H), 4.52-4.39 (m, 3H), 4.38- 4.31 (m, 2H), 3.54 (br d, J = 1.2 Hz, 2H), 3.46 (br d, J = 17.2 Hz, 2H), 3.29-3.16 (m, 2H), 2.56-2.30 (m, 2H), 2.28-2.10 (m, 3H), 2.08- 1.92 (m, 1H), 1.42-1.35 (m, 3H), 1.31 (br d, J = 6.4 Hz, 3H). |
| 131 | 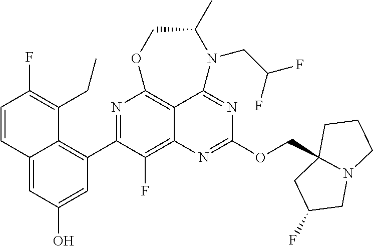 [SMILES: CCc1c(F)ccc2cc(O)cc(-c3nc4c5c(nc(OC[C@@]67CCCN6C[C@H](F)C7)nc5c3F)N(CC(F)F)[C@@H](C)CO4)c12] | ¹H NMR (400 MHz, MeOD- d₄) δ = 7.71-7.60 (m, 1H), 7.33-7.16 (m, 2H), 7.13- 6.97 (m, 1H), 6.63-6.23 (m, 1H),5.41-5.18 (m, 1H), 4.72-4.61 (m, 1H), 4.61- 4.42 (m, 3H), 4.36-4.23 (m, 3H), 4.19-4.04 (m, 1H), 3.26-3.18 (m, 2H), 3.09- 2.95 (m, 1H), 2.62-2.32 (m, 2H), 2.31-2.19 (m, 2H), 2.16-2.05 (m, 1H), 2.05- 1.96 (m, 2H), 1.93-1.82 (m, 1H), 1.40 (d, J = 6.8 Hz, 3H), 0.92-0.85 (m, 3H). |
| 132 | 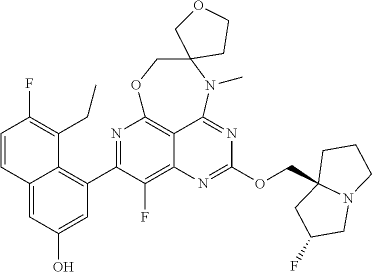 [SMILES: CCc1c(F)ccc2cc(O)cc(-c3nc4c5c(nc(OC[C@@]67CCCN6C[C@H](F)C7)nc5c3F)N(C)C3(CCOC3)CO4)c12] | ¹H NMR (400 MHz, MeOD- d₄) δ = 7.65 (dd, J = 5.6, 9.2 Hz, 1H), 7.30-7.20 (m, 2H), 7.06 (t, J = 2.0 Hz, 1H), 5.39- 5.22 (m, 1H), 4.58 (s, 2H), 4.37-4.25 (m, 2H), 4.23- 4.11 (m, 2H), 4.01-3.79 (m, 2H), 3.41 (d, J = 1.6 Hz, 3H), 3.28-3.17 (m, 3H), 3.07-2.97 (m, 1H), 2.54- 2.40 (m, 2H), 2.40-2.32 (m, 1H), 2.29-2.19 (m, 3H), 2.18-2.09 (m, 1H), 2.04- 1.96 (m, 2H), 1.95-1.84 (m, 1H), 0.86 (q, J = 6.8 Hz, 3H). |
| 133 | 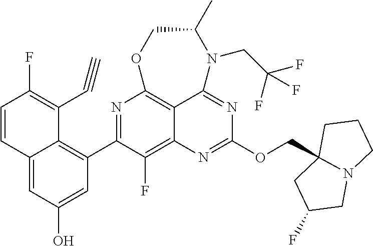 [SMILES: C#Cc1c(F)ccc2cc(O)cc(-c3nc4c5c(nc(OC[C@@]67CCCN6C[C@H](F)C7)nc5c3F)N(CC(F)(F)F)[C@@H](C)CO4)c12] | ¹H NMR (400 MHz, MeOH- d₄) δ = 7.90-7.81 (m, 1H), 7.37-7.15 (m, 1H), 5.50- 5.33 (m, 1H), 5.51-5.32 (m, 1H), 5.23-5.06 (m, 1H), 4.75-4.67 (m, 1H), 4.63- 4.45 (m, 4H), 4.43-4.31 (m, 2H), 3.64-3.48 (m, 3H), 3.44 (br s, 1H), 3.26-3.16 (m, 1H), 2.54-2.31 (m, 2H), 2.28-2.09 (m, 3H), 2.07- 1.92 (m, 1H), 1.45-1.38 (m, 3H). |

### Screen Assays

### HTRF KRAS Mutation Nucleotide Exchange Assays:

The HTRF KRAS nucleotide exchange assays are performed at Reaction Biology. Briefly, purified GST tagged KRAS G12C, KRAS G12D, KRAS G12V, KRAS G12R, KRAS G12S, KRAS G12D/T35S or KRAS WT proteins are mixed with a-GST Tb antibody in reaction buffer (20 mM Hepes, pH 7.4, 150 mM NaCl, 5 mM MgCl₂, 1 M DTT, 0.05% BSA, 0.0025% NP40). Serial dilutions of indicated compounds are prepared in DMSO and added to the KRAS/a-GST Th antibody mixture using acoustic dispenser (ECHO, Beckman). Compounds are incubated with KRAS/a-GST Tb antibody at room temperature for one hour. Purified SOS1 proteins and GTP-DY-647P1 mix is introduced to initiate the exchange reaction. HTRF signals are quantified 20-60 minutes later. The background subtracted signals are converted to % activity relative to DMSO controls. Data is analyzed using GraphPad Prism 4 with “sigmoidal dose-response (variable slope) equation to obtain compound IC₅₀ values.

| | IC₅₀ (nM) | | | | | | |
|---|---|---|---|---|---|---|---|
| Ex. | KRAS | KRAS | KRAS | KRAS | KRAS | KRAS | KRAS |
| # | G12C | G12D | G12V | G12R | G12S | G12D/T35S | WT |
| 1 | 0.57 | 2.88 | 1.79 | 0.51 | 0.88 | 0.67 | 1.30 |
| 2 | | 3.96 | 1.83 | | | | |
| 3 | | 1.66 | 1.85 | | | | |
| 25 | | 2.03 | 2.16 | | | | |
| 27 | | 1.17 | 1.00 | | | | |
| 28 | | 107.50 | 80.68 | | | | |
| 29 | | 1.60 | 1.78 | | | | |
| 30 | | 4.26 | 5.72 | | | | |
| 32 | | 3.20 | 2.68 | | | | |
| 33 | | 3.79 | 3.65 | | | | |
| 35 | | 21.77 | 8.87 | | | | |
| 40 | | 30.14 | 28.22 | | | | |
| 41 | | 2.84 | 2.58 | | | | |
| 43 | | 16.29 | 12.67 | | | | |
| 44 | | >10000 | >10000 | | | | |
| 45 | | 3.28 | 2.33 | | | | |
| 46 | | 2.10 | 1.71 | | | | |
| 47 | | 2.33 | 2.40 | | | | |
| 48 | | 1.40 | 2.10 | | | | |
| 50 | | 1.71 | 1.99 | | | | |
| 51 | | 1.86 | 1.87 | | | | |
| 52 | | 2.26 | 2.22 | | | | |
| 52 | | 2.26 | 2.22 | | | | |
| 53 | | 2.78 | 3.07 | | | | |
| 58 | | 1.79 | 1.82 | | | | |
| 59 | | 2.66 | 3.08 | | | | |
| 74 | | 4.00 | 2.81 | | | | |
| 75 | | 1.76 | 1.60 | | | | |
| 78 | | 40.17 | 51.94 | | | | |
| 79 | | 1.65 | 1.69 | | | | |
| 80 | | 8.30 | 3.75 | | | | |
| 81 | | 224.30 | 85.50 | | | | |
| 82 | | 2.64 | 2.79 | | | | |
| 83 | | 60.43 | 59.26 | | | | |
| 83 | | 60.43 | 59.26 | | | | |
| 84 | | 54.53 | 48.05 | | | | |
| 84 | | 54.53 | 48.05 | | | | |
| 85 | | 2.35 | 2.17 | | | | |
| 86 | | 3.88 | 3.24 | | | | |
| 87 | | 4.40 | 2.85 | | | | |
| 89 | | 3.93 | 4.20 | | | | |
| 90 | | 1.91 | 1.75 | | | | |
| 91 | | 14.94 | 14.98 | | | | |
| 92 | | 5.09 | 4.14 | | | | |
| 93 | | 1.99 | 1.55 | | | | |
| 94 | | 4.97 | 5.90 | | | | |
| 95 | | 3.79 | 3.15 | | | | |
| 96 | | 2.39 | 2.46 | | | | |
| 97 | | 1.51 | 1.73 | | | | |
| 98 | | 2.54 | 2.89 | | | | |
| 98 | | 2.54 | 2.89 | | | | |
| 99 | | 0.85 | 1.02 | | | | |
| 100 | | 12.05 | 9.93 | | | | |
| 101 | | 1.38 | 1.31 | | | | |
| 102 | | 379.00 | 364.40 | | | | |
| 103 | | 22.37 | 15.93 | | | | |
| 104 | | 7.51 | 9.01 | | | | |
| 105 | | 3.85 | 4.76 | | | | |
| 106 | | 3.26 | 3.83 | | | | |
| 107 | | 10.47 | 6.97 | | | | |
| 108 | | 1.50 | 1.92 | | | | |
| 109 | | 15.88 | 27.05 | | | | |
| 110 | | >10000 | >10000 | | | | |
| 111 | | 1.28 | 1.41 | | | | |
| 112 | | 321.10 | 40.06 | | | | |
| 113 | | 3827.00 | 4336.00 | | | | |
| 114 | | 929.00 | 1204.00 | | | | |
| 115 | | 2.61 | 2.40 | | | | |
| 116 | | 14.21 | 6.41 | | | | |
| 117 | | 28.78 | 28.46 | | | | |
| 118 | | 52.51 | 58.87 | | | | |
| 119 | | 2.48 | 2.16 | | | | |
| 120 | | 3.09 | 3.91 | | | | |
| 121 | | 1.74 | 1.99 | | | | |
| 122 | | 14.81 | 16.90 | | | | |
| 123 | | 2.13 | 2.34 | | | | |
| 124 | | 4.00 | 3.24 | | | | |
| 126 | | 110.80 | 30.19 | | | | |
| 127 | | 2.61 | 3.20 | | | | |

### Cell Proliferation Assays:

Two thousand KRAS mutant cells per well were seeded in 384-well white plate and then treated with indicated compounds for 72 hours at 37° C. and 5% CO₂. Cell proliferation was measured using CellTiter-Glo 2.0 luciferase-based ATP detection assay (Promega, Madison, WI) following the manufacturer's protocol. IC₅₀ values were determined using Prism software (GraphPad Software, San Diego, CA).

| | GP2D | SW620 | |
|---|---|---|---|
| | (KRAS | (KRAS | AGS |
| Ex. # | G12D) | G12V) | (G12D) |
| 1 | 2.51 | 10.43 | 6.90 |
| 2 | 17.17 | 41.37 | 52.31 |
| 3 | 15.53 | | 57.50 |
| 25 | 76.41 | 654.20 | 165.60 |
| 27 | >3000 | | >3000 |
| 28 | >3000 | >3000 | >3000 |
| 29 | 363.70 | >3000 | 760.40 |
| 30 | 50.51 | 139.40 | 97.27 |
| 31 | 1884.00 | >3000 | >3000 |
| 32 | 292.90 | 693.40 | 622.70 |
| 33 | 962.00 | 1532.00 | 2838.00 |
| 35 | >3000 | >3000 | >3000 |
| 40 | >3000 | >3000 | >3000 |
| 41 | 20.71 | 41.98 | 43.06 |
| 43 | 2123.00 | >3000 | >3000 |
| 44 | >3000 | >3000 | >3000 |
| 45 | 361.10 | 320.80 | 620.70 |
| 46 | 74.82 | 414.15 | 192.53 |
| 47 | 6000.00 | >3000 | 788.80 |
| 48 | 2195.21 | >3000 | >3000 |
| 50 | 8.94 | 25.73 | 28.45 |
| 51 | 215.80 | 2269.00 | >3000 |
| 52 | 1.75 | 9.20 | 7.70 |
| 52 | 1.75 | 9.20 | 7.70 |
| 53 | 34.54 | | 113.20 |
| 56 | 2.45 | 3.51 | 5.14 |
| 58 | 36.70 | 43.15 | 59.44 |
| 59 | 12.44 | 77.23 | 70.62 |
| 74 | 480.30 | 348.50 | 1484.00 |
| 75 | 349.03 | 702.09 | 1617.34 |
| 76 | 3.35 | 11.62 | 12.30 |
| 77 | 1354.00 | >3000 | >3000 |
| 78 | >3000 | >3000 | >3000 |
| 79 | 2.26 | 11.15 | 3.33 |
| 80 | >3000 | >3000 | >3000 |
| 81 | >3000 | >3000 | >3000 |
| 82 | 470.50 | >3000 | 1441.00 |
| 83 | 1083.00 | >3000 | >3000 |
| 83 | 1083.00 | >3000 | >3000 |
| 84 | >3000 | >3000 | >3000 |
| 84 | >3000 | >3000 | >3000 |
| 85 | 143.40 | 820.00 | 631.40 |
| 86 | 9.10 | 104.70 | 28.31 |
| 87 | 64.54 | 686.50 | 132.50 |
| 89 | 1171.00 | >3000 | >3000 |
| 90 | 188.70 | 1953.00 | 754.40 |
| 91 | 542.95 | >3000 | 923.39 |
| 92 | 347.67 | 579.07 | 392.64 |
| 93 | 140.30 | 981.40 | 560.20 |
| 94 | 1103.00 | >3000 | >3000 |
| 95 | 992.60 | >3000 | >3000 |
| 96 | 16.83 | 92.56 | 82.92 |
| 97 | 915.70 | 1175.00 | 385.10 |
| 98 | 114.19 | | |
| 98 | 114.19 | | |
| 99 | 22.35 | 96.76 | 86.32 |
| 100 | >3000 | >3000 | >3000 |
| 101 | 880.10 | >3000 | >3000 |
| 102 | >3000 | >3000 | >3000 |
| 103 | 580.10 | >3000 | 924.50 |
| 104 | 188.50 | 902.90 | 665.50 |
| 105 | 21.58 | 86.15 | 81.01 |
| 106 | 14.61 | 64.55 | 48.84 |
| 107 | 749.20 | 1039.79 | 1128.00 |
| 108 | 13.77 | 195.49 | 42.70 |
| 109 | 1996.00 | >3000 | >3000 |
| 110 | >3000 | >3000 | >3000 |
| 111 | 59.66 | 499.30 | 360.60 |
| 112 | >3000 | | >3000 |
| 113 | >3000 | | >3000 |
| 114 | >3000 | | >3000 |
| 115 | 106.50 | | 352.30 |
| 116 | 760.30 | | >3000 |
| 117 | >3000 | >3000 | >3000 |
| 118 | >3000 | >3000 | >3000 |
| 119 | 102.95 | 307.28 | 257.76 |
| 120 | >3000 | >3000 | >3000 |
| 121 | 3.67 | 16.63 | 21.51 |
| 122 | 2412.00 | 4896.00 | 5491.00 |
| 123 | 40.55 | 149.98 | 374.57 |
| 124 | 1755.00 | 3321.00 | 3314.00 |
| 126 | >3000 | 109.30 | >3000 |
| 127 | 4.54 | 9.86 | 8.80 |
| 128 | 5.805 | 6.307 | 14.6 |
| 129 | >3000 | >3000 | >3000 |
| 130 | 115.1 | 131.4 | 196.7 |
| 131 | 23.92 | 42.67 | 65.34 |
| 132 | >3000 | 1377 | >3000 |
| 133 | 21.29 | 25.75 | 49.86 |

Method B: Two thousand KRAS mutant cells per well were seeded in 96-well black plate and then treated with indicated compounds for 5 days at 37° C. and 5% CO₂. Cell proliferation was measured using CellTiter-Glo 2.0 luciferase-based ATP detection assay (Promega, Madison, WI) following the manufacturer's protocol. IC₅₀ values were determined using Prism software (GraphPad Software, San Diego, CA).

Anti-Cell
Proliferation
IC₅₀ (nM)

| Cell Line | KRAS Mutation | Ex. 1 | Ex. 2 |
|---|---|---|---|
| GP2D | G12D | 2.80 | 11.30 |
| AGS | G12D | 14.83 | 86.19 |
| HPAC | G12D | 18.9 | 94.5 |
| AsPC1 | G12D | 108.4 | 212.7 |
| PANC0403 | G12D | 27.7 | 76.7 |
| PANC0504 | G12D | 9.0 | 44.0 |
| H23 | G12C | 47.4 | 144.7 |
| H358 | G12C | 49.2 | 222.3 |
| MiaPaCa2 | G12C | 15.2 | 68.1 |
| SW620 | G12V | 9.6 | 43.8 |
| CAPAN-2 | G12V | 35.2 | 60.2 |
| H2291 | G12F | 5.70 | 37.24 |
| HCT116 | G13D | 473.6 | >3000 |

### Kinase Phosphorylation Assays:

KRAS mutant cells were plated in clear bottom 96 well plates at a density of 50,000-120,000 cells per well. Cells were allowed to attach overnight and then treated with compounds for 3 hours. After treatment, cells were fixed with 10% buffered formalin for 20 minutes at room temperature, washed with PBS, and then permeabilized with ice cold 100% methanol for 10 minutes to overnight at −20° C. Odyssey Blocking Buffer (LiCOR Biosciences: 927-60001) was added to each well for 1 hour at room temperature prior to incubation with primary antibodies overnight at 4° C. The primary antibodies used were as follows: Phospho-ERK (Cell Signaling: CS-9101) diluted to 1:250 and p-actin (Cell Signaling: CS-3700) diluted to 1:2000 in Odyssey Blocking Buffer+0.05% Tween20. Plates were washed 3× with Wash Buffer (PBS+0.1% Tween20) and incubated with secondary antibodies for 2 hours at room temperature. The following secondary antibodies were used: goat anti-rabbit-800 (LI-COR: 926-32211) and goat anti-mouse-680 (LICOR: 926-68070) both diluted to 1:2000 in Odyssey Blocking Buffer+0.05% Tween20. The plates were washed 3× with Wash Buffer before imaged on LiCOR Odyssey M Imaging System. Phospho-ERK signal was normalized to β-actin signal for each well and the percent of DMSO control values were calculated. IC₅₀ values were calculated using the sigmoidal dose-response (variable slope) equation in GraphPad Prism 4.

pERK ½
IC₅₀ (nM)

| Cell Line | KRAS Mutation | Ex. 1 | Ex. 2 |
|---|---|---|---|
| AGS | G12D | 3.46 | 19.58 |
| SW620 | G12V | 3.24 | 22.08 |
| H2291 | G12F | 4.29 | 65.03 |
| HCT116 | G13D | 14.06 | 93.42 |

What is claimed is:

1. A compound of the formula I, or a pharmaceutically acceptable salt thereof, 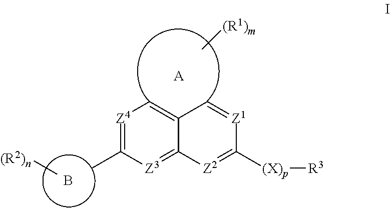
wherein
X is a —O—, —S—, or —NR¹—;
Z¹ is N or C(R⁵);
Z² is N or C(R⁶);
Z³ is N or C(R⁷);
Z⁴ is N or C(R⁸);
provided that at least two of Z¹-Z⁴ are N;
ring A is a 5- to 8-membered heterocycloalkyl or a C₅-C₅ cycloalkyl;
ring B is a C₆-C₁₀ aryl or 5- to 10-membered heteroaryl;
each R¹ is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R² is independently deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{c}, —OC(O)R^{c}, —OC(O)NR^{c}R^{d}, —OC(═NR^{c})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{c})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{c})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂, or two of R² taken together with the atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆ haloalkyl, —R^{e}, —R^{f}, —OR^{e}, —OC(O)R^{c}, —OC(O)NR^{c}R^{f}, —OS(O)R^{c}, —OS(O)₂R^{c}, —OS(O)NR^{c}R^{f}, —OS(O)₂NR^{c}R^{f}, —SR^{c}, —S(O)R^{e}, —S(O)₂R^{e}, —S(O)NR^{e}R^{f}, —S(O)₂NR^{e}R^{f}, —NR^{c}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R³ is —C₁-C₆ alkyl, —C₂-C₆ alkenyl, —C₂-C₆ alkynyl, —C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, or —C₁-C₆alkylene-(4- to 10-membered heterocycloalkyl), wherein each hydrogen atom in C₁-C₆ alkyl, C₂-C₆ alkenyl, C₂-C₆ alkynyl, C₃-C₆ cycloalkyl, 4- to 10-membered heterocycloalkyl, —C₁-C₆ alkylene-(4- to 10-membered heterocycloalkyl), C₆-C₁₀ aryl, —C₁-C₆ alkylene-(C₆-C₁₀ aryl), 5- to 10-membered heteroaryl, and —C₁-C₆ alkylene-(5- to 10-membered heteroaryl), is independently optionally substituted by deuterium, halogen, —R^{e}, —R^{f}, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁵, R⁶, R⁷, and R⁸ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R⁸, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
each R^{a}, R^{b}, R^{c}, R^{d}, R^{e}, R^{f}, R^{g}, and R^{h} is independently selected from the group consisting of H, deuterium, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl; or two of R^{a} and R^{b}, or R^{c} and R^{d}, or R^{e} and R^{f}, or R^{g} and R^{h}, taken together with the atom or atoms to which they are attached, combine to form a C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, C₁-C₆ alkylene-C₆-C₁₀ aryl, and 5- to 10-membered heteroaryl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or —R^{c} and —R^{f} taken together with the carbon atom to which they are attached form an oxo groups or an alkenyl;
m is 0, 1, 2, 3, 4, 5, 6, or 7;
n is 0, 1, 2, 3, 4, 5, 6, or 7; and
p is 0 or 1.

2. The compound of claim 1, or a pharmaceutically acceptable salt thereof, wherein the compound is of the formula II, 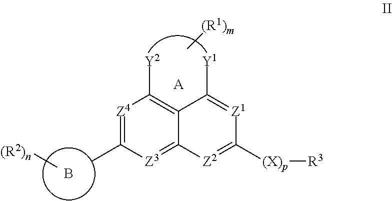
wherein
Y¹ is —O—, —S—, —S(O)—, —S(O)₂—, —NR^{g}— or —CR¹¹R¹²;
Y² is —O—, —S—, —S(O)—, —S(O)₂—, —NR¹⁰—, or —CR¹³R¹⁴—;
each R⁹ and R¹⁰ is independently H, deuterium, —C(O)R^{g}, —C(O)NR^{g}R^{h}, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{c}S(O)R^{f}, —NR^{c}S(O)₂R^{f}, —NR'S(O)NR^{c}R^{f}, —NR^{c}S(O)₂NR^{c}R^{f}, —C(O)R^{c}, —C(O)OR^{c}, —C(O)NR^{e}R^{f}, —PR^{c}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e′}, —CN, or —NO₂; or R^{g} and a R¹ or R¹⁰ and a R¹, taken together with the atoms to which they are attached, combine to form a 4- to 10-membered heterocycloalkyl, wherein each hydrogen atom in the 4- to 10-membered heterocycloalkyl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or two hydrogen atoms on a single carbon atom of the 4- to 10-membered heterocycloalkyl combine to form an oxo group or an alkenyl group; and
each of R¹¹, R¹², R¹³, and R¹⁴ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR¹, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{a}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or a R¹ and R¹¹, a R¹ and R¹², a R¹ and R¹³, a R¹ and R¹⁴, two of R¹ and R¹², or two of R¹³ and R¹⁴ taken together with the carbon or carbons to which they are attached, combine to form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, or two of R¹ and R¹² or R¹³ and R¹⁴ taken together with the carbon atom to which they are attached form an oxo group or an C₂-C₆ alkenyl group.

3. The compound of claim 1, or a pharmaceutically acceptable salt thereof, wherein the compound is of the formula XXXVII, XXXVIII, XXXIX, XXXX, XXXXI, XXXXII, or XXXXIII 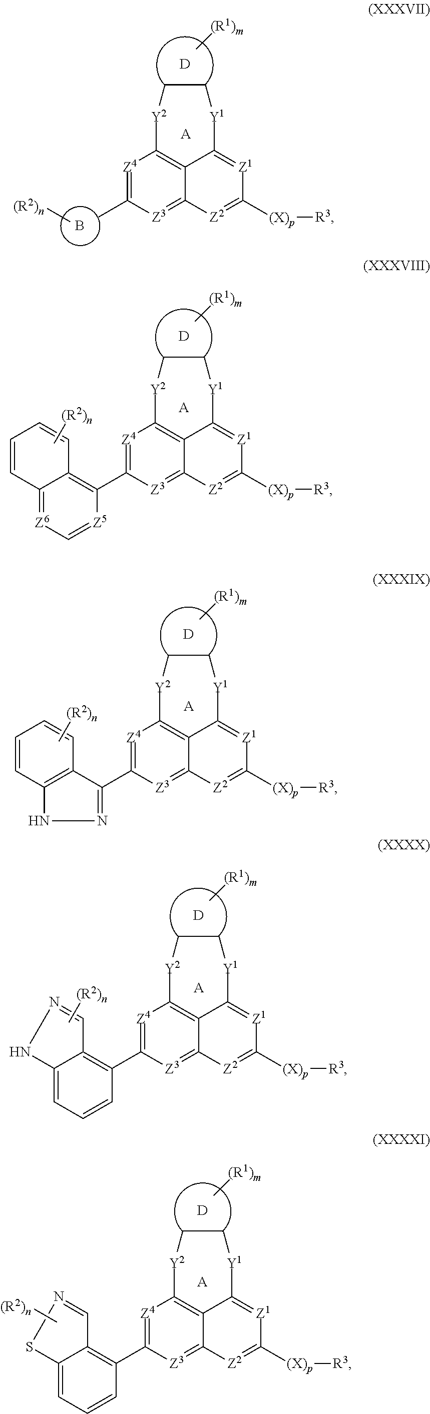 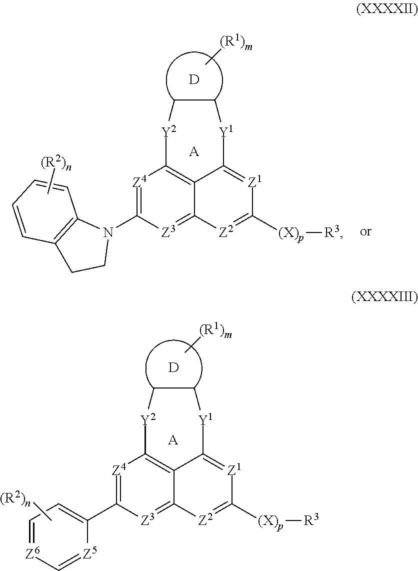
wherein
Y¹ is —O—, —S—, —S(O)—, —S(O)₂—, —NR^{g}— or —CR¹¹R¹²—;
Y² is —O—, —S—, —S(O)—, —S(O)₂—, —NR¹⁰—, or —CR¹³R¹⁴—;
Z⁵ is N or C(R¹⁵);
Z⁶ is N or C(R¹⁶) ring D is a C₃-C₆ cycloalkyl or a 4- to 8-membered heterocycloalkyl;
each R⁹ and R¹⁰ is independently H, deuterium, —C(O)R^{g}, —C(O)NR^{g}R^{h}, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, and 5- to 10-membered heteroaryl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R⁹ and a R¹ or R¹⁰ and a R¹, taken together with the atoms to which they are attached, combine to form a 4- to 10-membered heterocycloalkyl, wherein each hydrogen atom in the 4- to 10-membered heterocycloalkyl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂, or two hydrogen atoms on a single carbon atom of the 4- to 10-membered heterocycloalkyl combine to form an oxo group or a C₂-C₆ alkenyl group;
each of R¹¹, R¹², R¹³, and R¹⁴ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{a}, —OC(O)R^{a}, —OC(O)NR^{a}R^{b}, —OC(═NR^{a})NR^{a}R^{b}, —OS(O)R^{a}, —OS(O)₂R^{a}, —OS(O)NR^{a}R^{b}, —OS(O)₂NR^{a}R^{b}, —SR^{a}, —S(O)R^{a}, —S(O)₂R^{a}, —S(O)NR^{a}R^{b}, —S(O)₂NR^{a}R^{b}, —NR^{a}R^{b}, —NR^{a}C(O)R^{b}, —N(C(O)R^{a})(C(O)R^{b}), —NR^{a}C(O)OR^{b}, —NR^{a}C(O)NR^{a}R^{b}, —NR^{c}C(═NR^{a})NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(═NR^{a})NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, or a R¹ and R¹¹, a R¹ and R¹², a R¹ and R¹³, a R¹ and R¹⁴, two of R^{1′} and R¹², or two of R¹³ and R¹⁴ taken together with the carbon or carbons to which they are attached, combine to form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, or two of R¹ and R¹² or R¹³ and R¹⁴ taken together with the carbon atom to which they are attached form an oxo group or an C₂-C₆ alkenyl group; and
each of R¹⁵ and R¹⁶ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{e}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂.

4. The compound of claim 1 or 2, wherein the compound is of the formula III 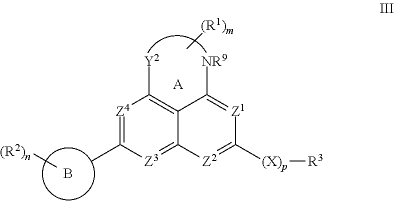
or a pharmaceutically acceptable salt thereof.

5. The compound of claim 1 or 2, or a pharmaceutically acceptable salt thereof, wherein the compound is of the formula XVI, XXIII, or XXX, 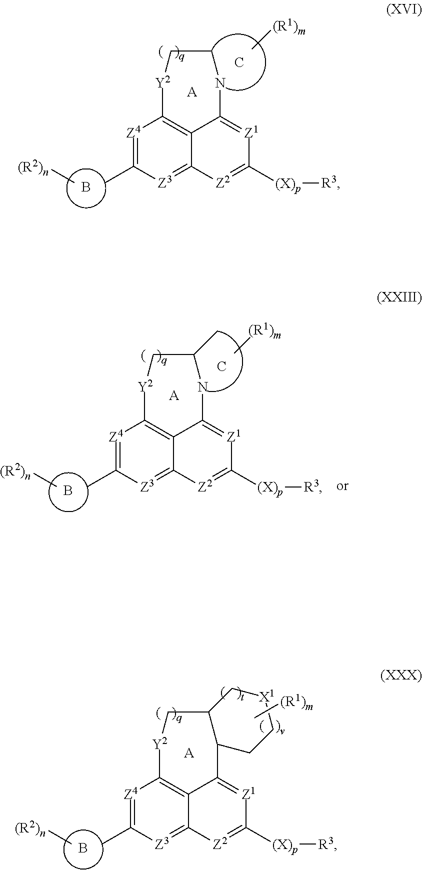
wherein ring C is a 4- to 8-membered heterocycloalkyl, X¹ is —NH—, —O— or —CH₂—, q is 0, 1, or 2, t is 1 or 2, and v is 1 or 2.

6. The compound of any one of claims 1 to 5, or a pharmaceutically acceptable salt thereof, wherein the compound is of the formula IV, XIV, XVII, XXII, XXIV, XXIX, XXXI, or XXXVI, 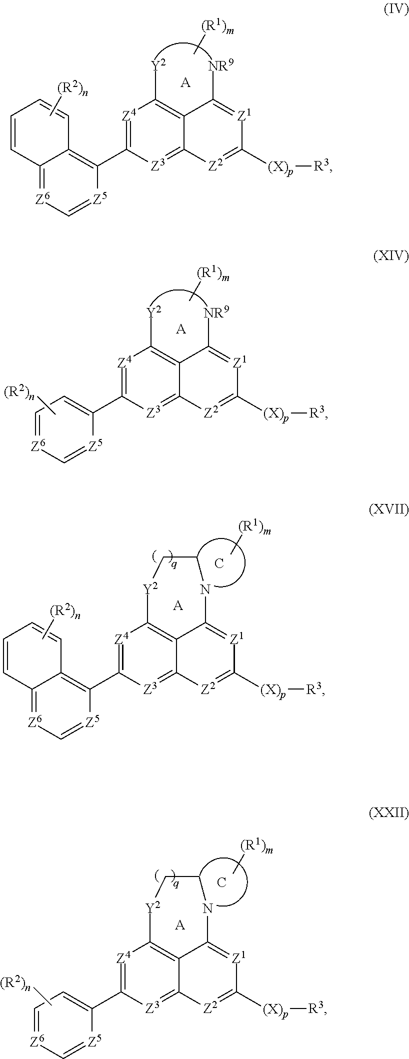 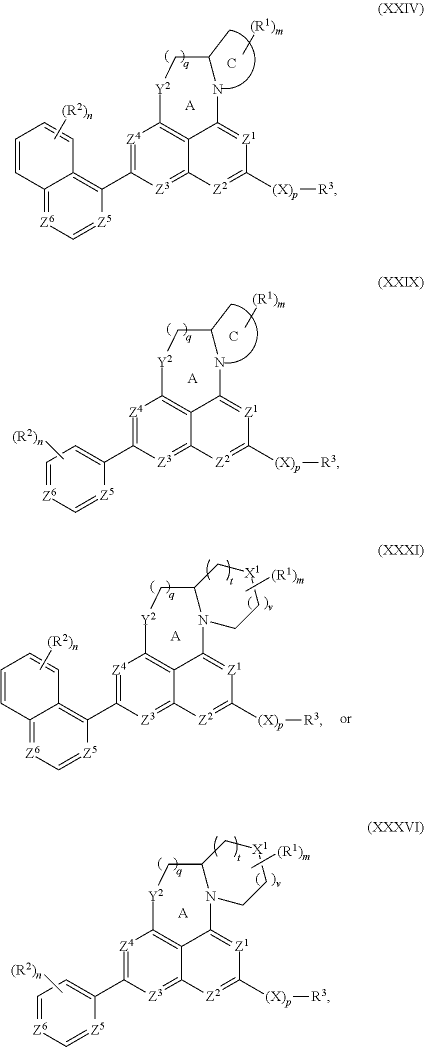
Z⁵ is N or C(R¹⁵);
Z⁶ is N or C(R¹⁶);
each of R¹⁵ and R¹⁶ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂;
ring C is a 4- to 8-membered heterocycloalkyl;
X¹ is —O—, —NH—, or —CH₂—;
q is 0, 1, or 2;
t is 1 or 2, and
v is 1 or 2.

7. The compound of any one of claims 1 to 5, or a pharmaceutically acceptable salt thereof, wherein the compound is of the formula V, XI, XVIII, XXI, XXV, XXVIII, XXXII, or XXXV, 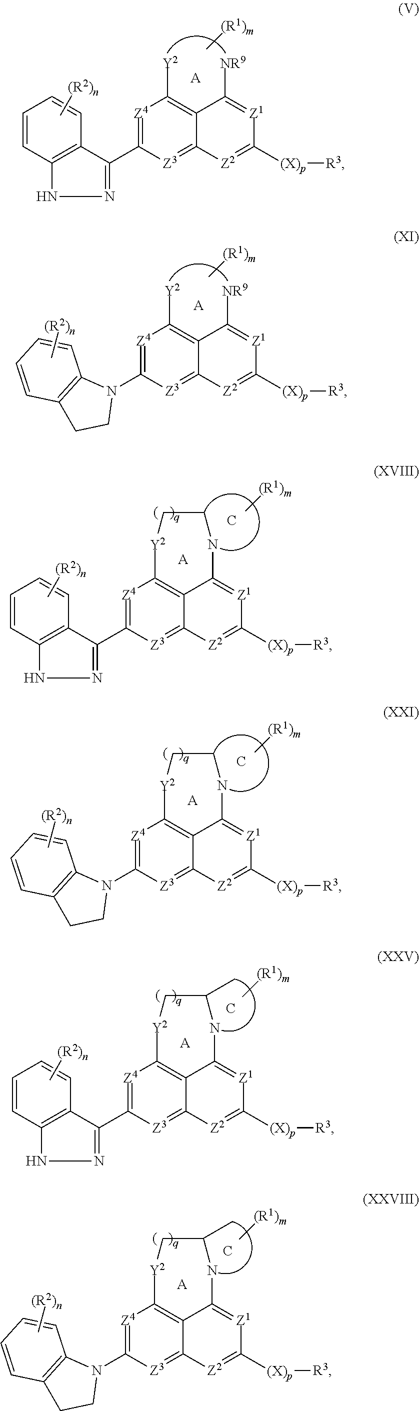 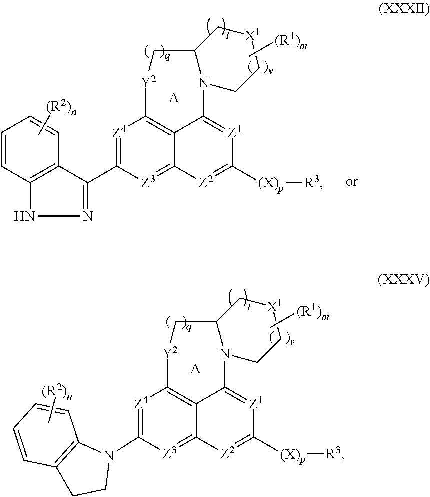
wherein ring C is a 4- to 8-membered heterocycloalkyl; X¹ is —O—, —NH—, or —CH₂—; q is 0, 1, or 2; t is 1 or 2; and v is 1 or 2.

8. The compound of any one of claims 1 to 5, or a pharmaceutically acceptable salt thereof, wherein the compound is of the formula VI, X, XIX, XX, XXVI, XXVII, XXXIII, XXXIV, XXXXIII, XXXXIV, XXXXV, or XXXXVI, 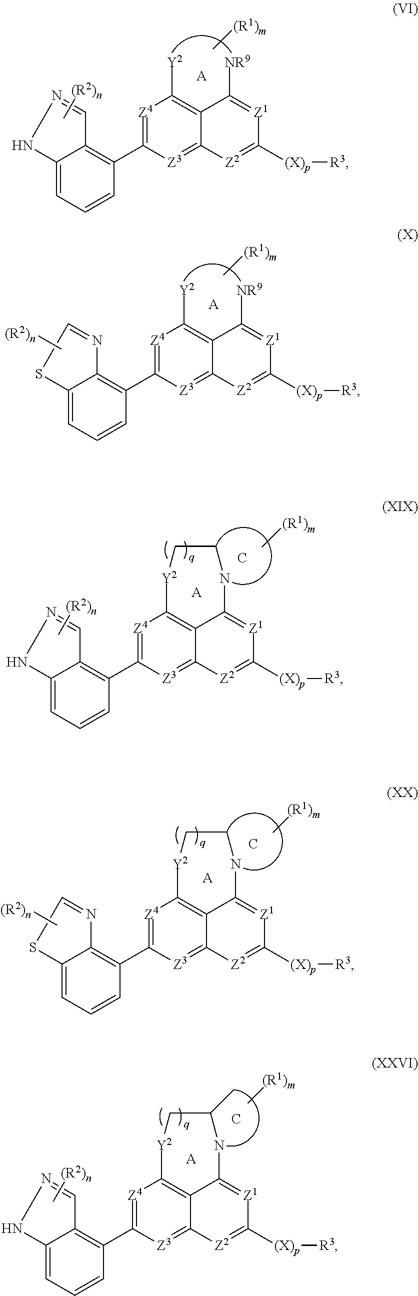 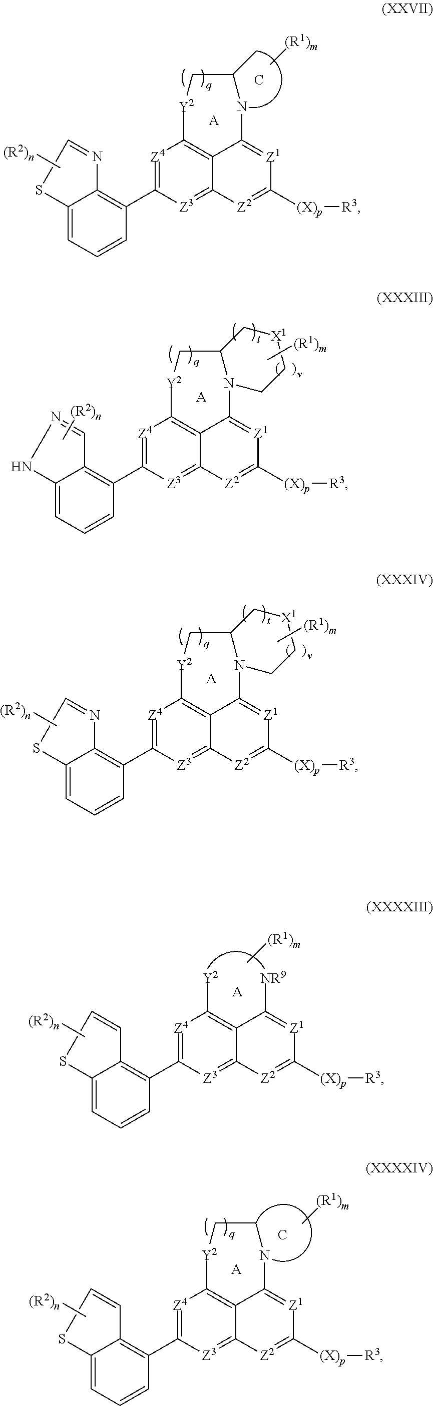 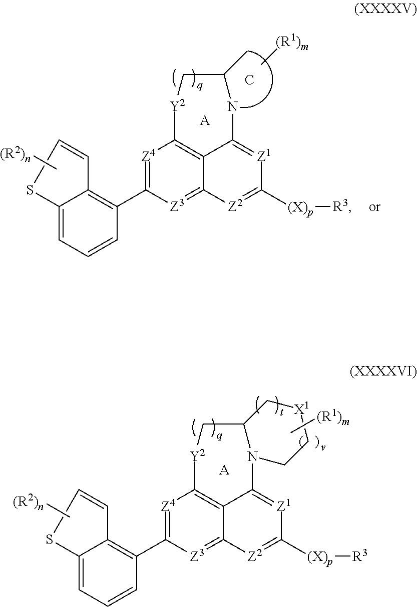
wherein ring C is a 4- to 8-membered heterocycloalkyl; X¹ is —O—, —NH— or —CH₂—; q is 0, 1, or 2; t is 1 or 2; and v is 1 or 2.

9. The compound of any one of claims 1 to 5, or a pharmaceutically acceptable salt thereof, wherein the compound is of the formula VII or XV, 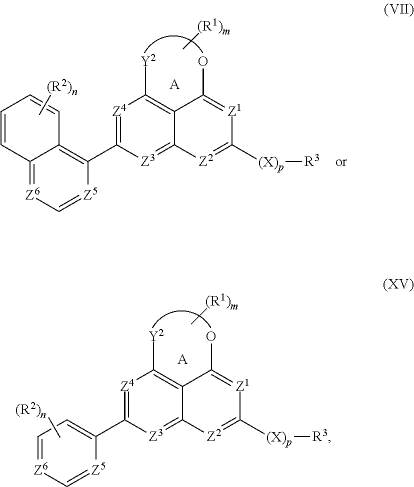
Z⁵ is N or C(R¹⁵);
Z⁶ is N or C(R¹⁶); and
each of R¹⁵ and R¹⁶ is independently H, deuterium, halogen, C₁-C₆ alkyl, C₂-C₆ alkenyl, C₂-C₆ alkynyl, C₃-C₆ cycloalkyl, 4- to 10-membered heterocycloalkyl, C₆-C₁₀ aryl, 5- to 10-membered heteroaryl, —OR^{g}, —OC(O)R^{g}, —OC(O)NR^{g}R^{h}, —OS(O)R^{g}, —OS(O)₂R^{g}, —SR^{g}, —S(O)R^{g}, —S(O)₂R^{g}, —S(O)NR^{g}R^{h}, —S(O)₂NR^{g}R^{h}, —OS(O)NR^{g}R^{h}, —OS(O)₂NR^{g}R^{h}, —NR^{g}R^{h}, —NR^{g}C(O)R^{h}, —NR^{g}C(O)OR^{h}, —NR^{g}C(O)NR^{g}R^{h}, —NR^{g}S(O)R^{h}, —NR^{g}S(O)₂R^{h}, —NR^{g}S(O)NR^{g}R^{h}, —NR^{g}S(O)₂NR^{g}R^{h}, —C(O)R^{g}, —C(O)OR^{g}, —C(O)NR^{g}R^{h}, —PR^{g}R^{h}, —P(O)R^{g}R^{h}, —P(O)₂R^{g}R^{h}, —P(O)NR^{g}R^{h}, —P(O)₂NR^{g}R^{h}, —P(O)OR^{g}, —P(O)₂OR^{g}, —CN, or —NO₂.

10. The compound of any one of claims 1 to 5, or a pharmaceutically acceptable salt thereof, wherein the compound is of the formula VIII or XIII 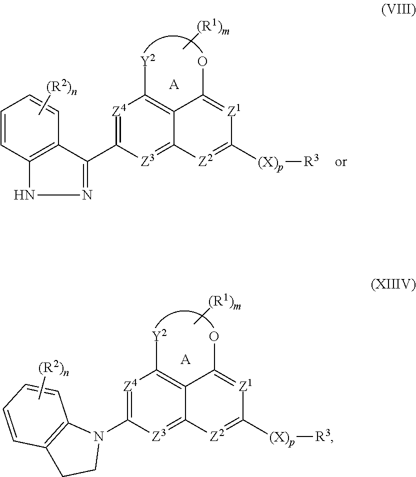
or a pharmaceutically acceptable salt thereof.

11. The compound of any one of claims 1 to 5, or a pharmaceutically acceptable salt thereof, wherein the compound is of the formula IX or XII 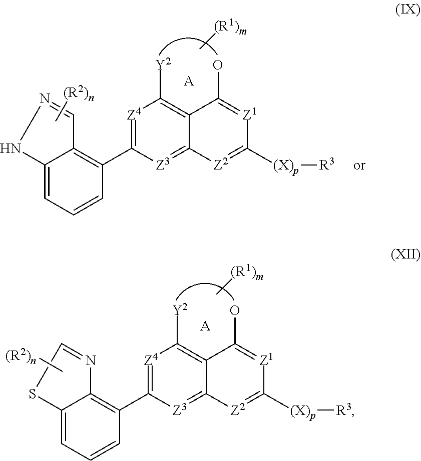
or a pharmaceutically acceptable salt thereof.

12. The compound of any one of the preceding claims, or a pharmaceutically acceptable salt thereof, wherein R¹ is deuterium, C₁-C₆ alkyl, C₂-C₆ alkenyl, or C₂-C₆ alkynyl, wherein each hydrogen atom in C₁-C₆ alkyl, C₂-C₆ alkenyl, or C₂-C₆ alkynyl is independently optionally substituted by deuterium, halogen, —R^{e}, —R^{f}, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; R^{g} and a R¹ or R¹⁰ and a R¹, taken together with the atoms to which they are attached, combine to form a 4- to 10-membered heterocycloalkyl, wherein each hydrogen atom in the 4- to 10-membered heterocycloalkyl is independently optionally substituted by deuterium, halogen, C₁-C₆ alkyl, C₁-C₆ haloalkyl, —OH, —OC₁-C₆ alkyl, —OC(O)C₁-C₆ alkyl, —OC(O)N(H or C₁-C₆ alkyl)₂, —OS(O)C₁-C₆ alkyl, —OS(O)₂C₁-C₆ alkyl, —OS(O)N(H or C₁-C₆ alkyl)₂, —OS(O)₂N(H or C₁-C₆ alkyl)₂, —SC₁-C₆ alkyl, —S(O)C₁-C₆ alkyl, —S(O)₂C₁-C₆ alkyl, —S(O)N(H or C₁-C₆ alkyl)₂, —S(O)₂N(H or C₁-C₆ alkyl)₂, —N(H or C₁-C₆ alkyl)₂, —N(C₁-C₆ alkyl)C(O)—C₁-C₆ alkyl, —N(C₁-C₆ alkyl)C(O)OC₁-C₆ alkyl, —N(C₁-C₆ alkyl)C(O)N(H or C₁-C₆ alkyl)₂, —N(C₁-C₆ alkyl)S(O)C₁-C₆ alkyl, —N(C₁-C₆ alkyl)S(O)₂C₁-C₆ alkyl, —N(C₁-C₆ alkyl)S(O)N(H or C₁-C₆ alkyl)₂, —N(C₁-C₆ alkyl)S(O)₂N(H or C₁-C₆ alkyl)₂, —C(O)C₁-C₆ alkyl, —C(O)OC₁-C₆ alkyl, —C(O)N(H or C₁-C₆ alkyl)₂, —P(H or C₁-C₆ alkyl)₂, —P(O)(H or C₁-C₆ alkyl)₂, —P(O)₂ (H or C₁-C₆ alkyl)₂, —P(O)N(H or C₁-C₆ alkyl)₂, —P(O)₂N(H or C₁-C₆ alkyl)₂, —P(O)OC₁-C₆ alkyl, —P(O)₂OC₁-C₆ alkyl, —CN, or —NO₂; or two hydrogen atoms on a single carbon atom of the 4- to 10-membered heterocycloalkyl combine to form an oxo group or an C₂-C₆ alkenyl group; or two of R¹ taken together with the atom or atoms to which they are attached form a C₃-C₆ cycloalkyl or a 4- to 10-membered heterocycloalkyl, wherein each hydrogen atom in C₁-C₆ alkyl, C₂-C₆ alkenyl, C₂-C₆ alkynyl, C₃-C₆ cycloalkyl, C₆-C₁₀ aryl, 5- to 10-membered heteroaryl, and 4- to 10-membered heterocycloalkyl is independently optionally substituted by deuterium, halogen, C₁-C₆ alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

13. The compound of any one of the preceding claims, or a pharmaceutically acceptable salt thereof, wherein ring A is optionally taken together with ring C or ring D to form a core of the compound of the formula I, the core having the formula 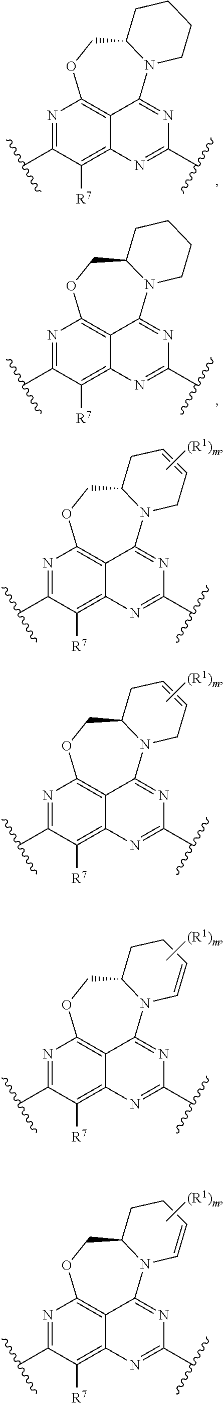 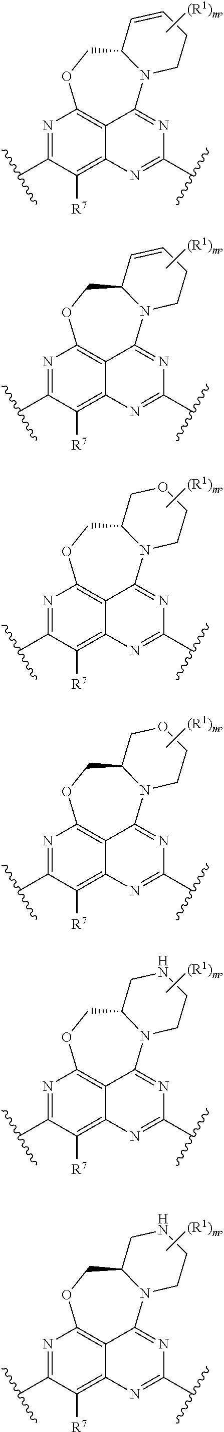 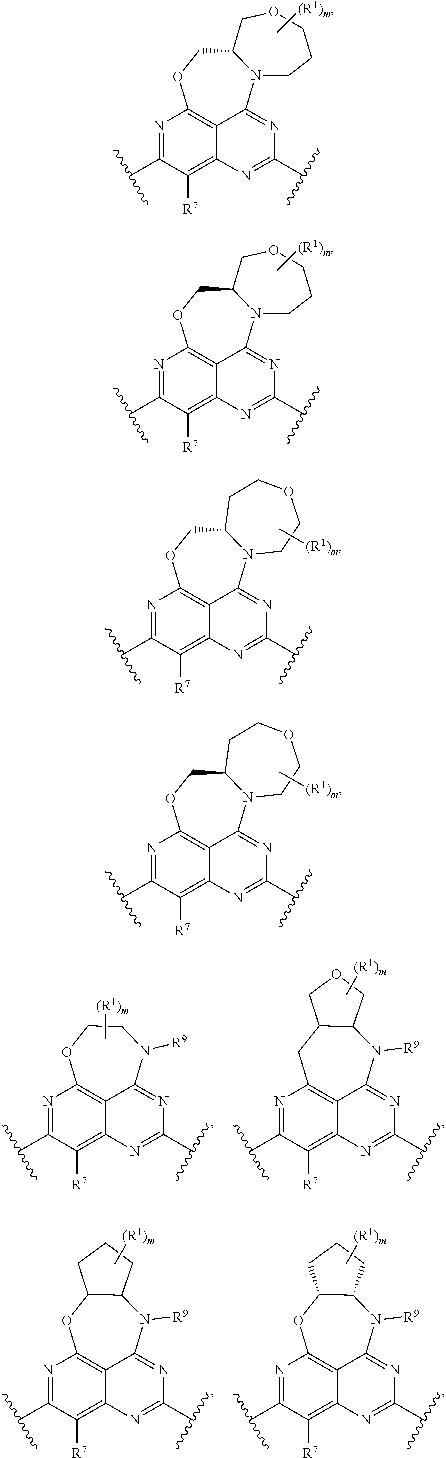 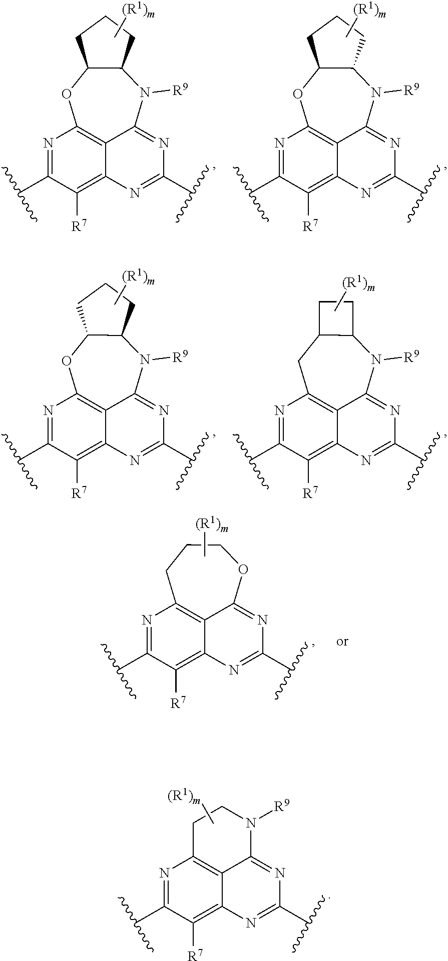
wherein each “ [symbol] ” is a point of covalent attachment to either ring B or —(X)ₚ—R³, and each hydrogen atom in ring A, ring C, if present, and ring D, if present, is independently optionally substituted with an R¹ selected from the group consisting of deuterium, halogen, C₁-C₆ alkyl, C₁-C₆haloalkyl, —R^{e}, —R^{f}, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

14. The compound of any one of the preceding claims, or a pharmaceutically acceptable salt thereof, wherein ring A is optionally taken together with ring C or ring D to form core of the compound of the formula I, the core having the formula 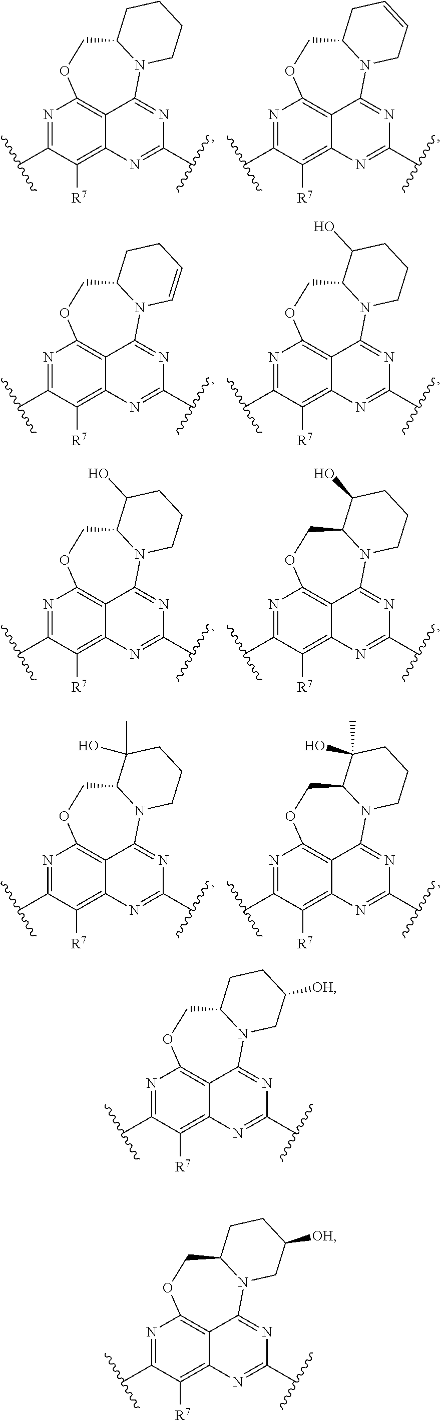 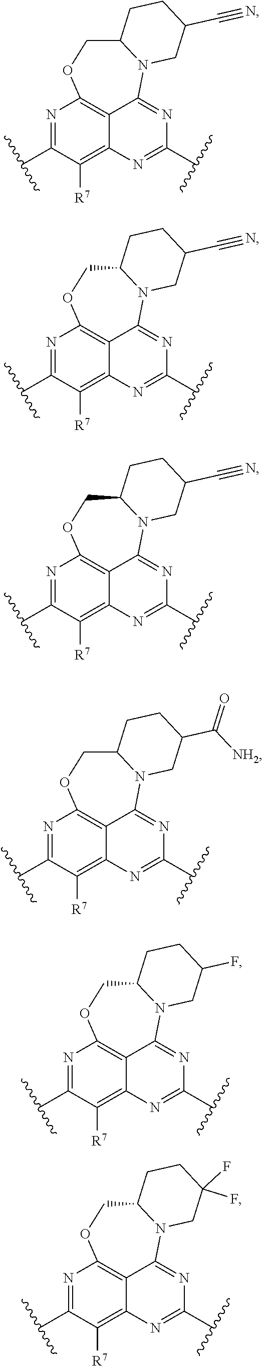 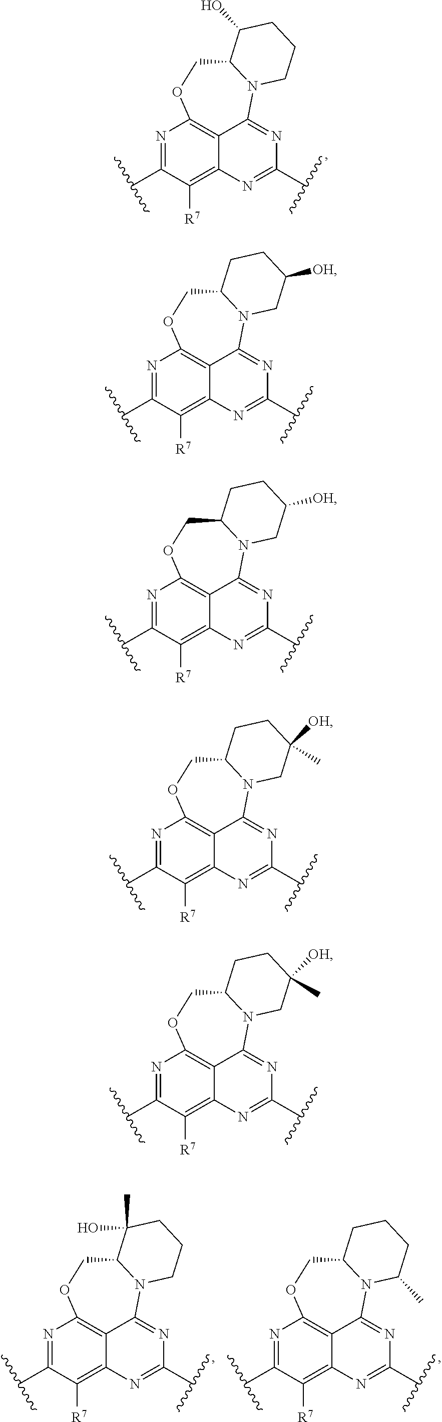 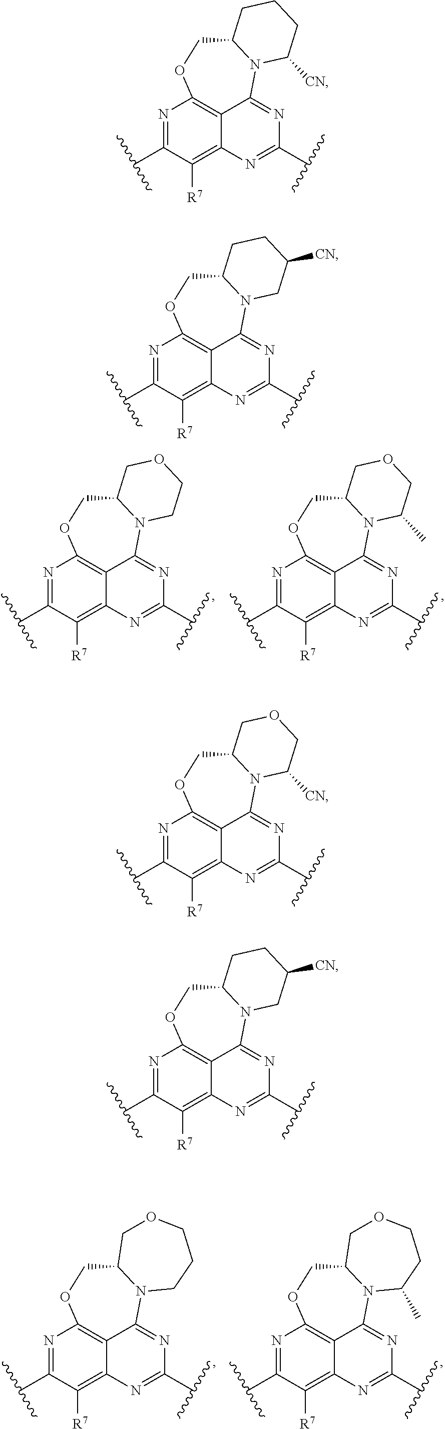 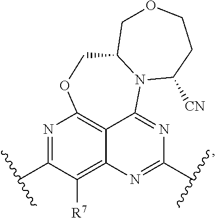 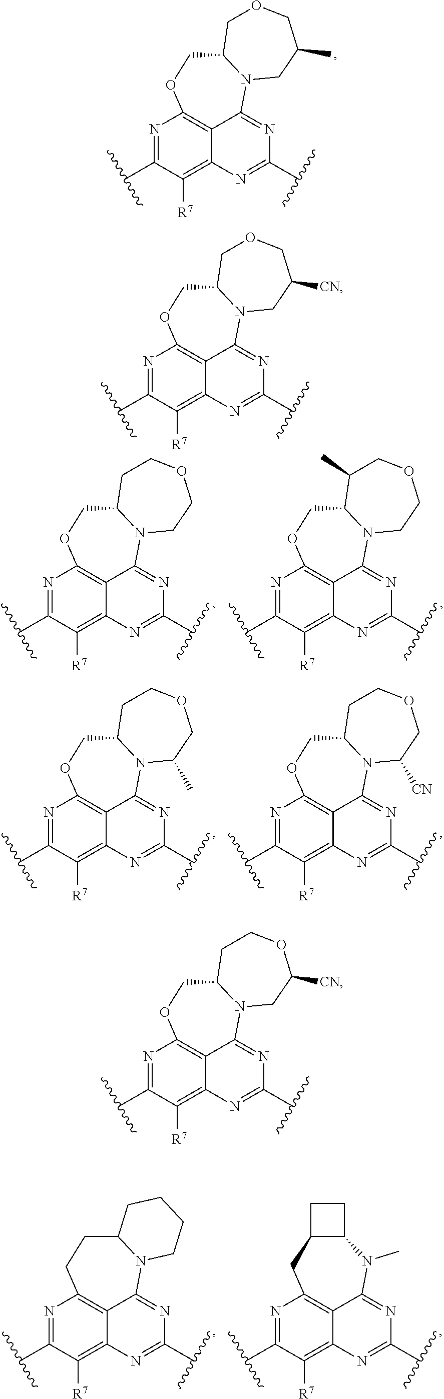 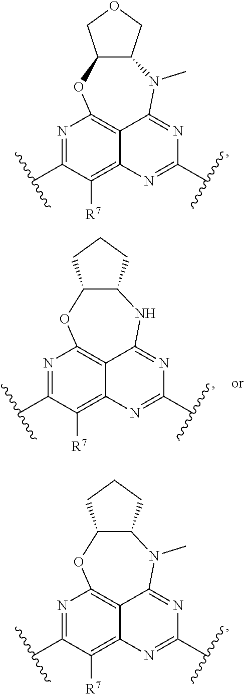
wherein each wherein “ [symbol] ” is a point of covalent attachment to either ring B or —(X)ₚ—R³.

15. The compound of any one of claims 1 to 13, or a pharmaceutically acceptable salt thereof, wherein ring A is a fused 5- to 8-membered heterocycloalkyl that forms a core of the compound of the formula I, the core having the formula 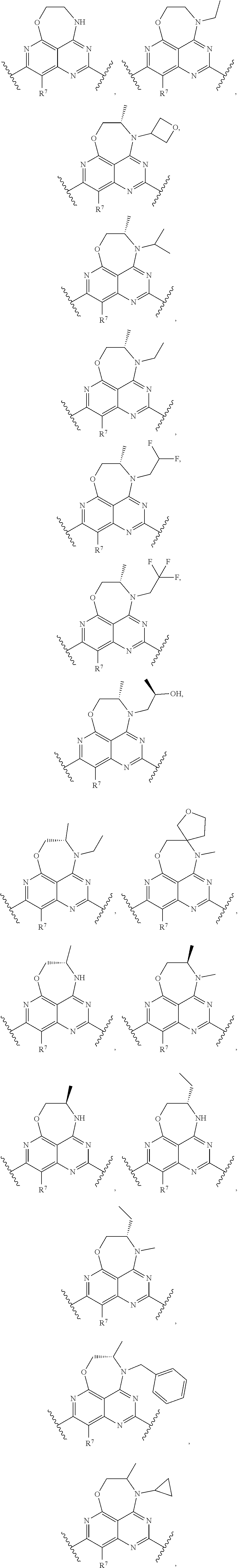 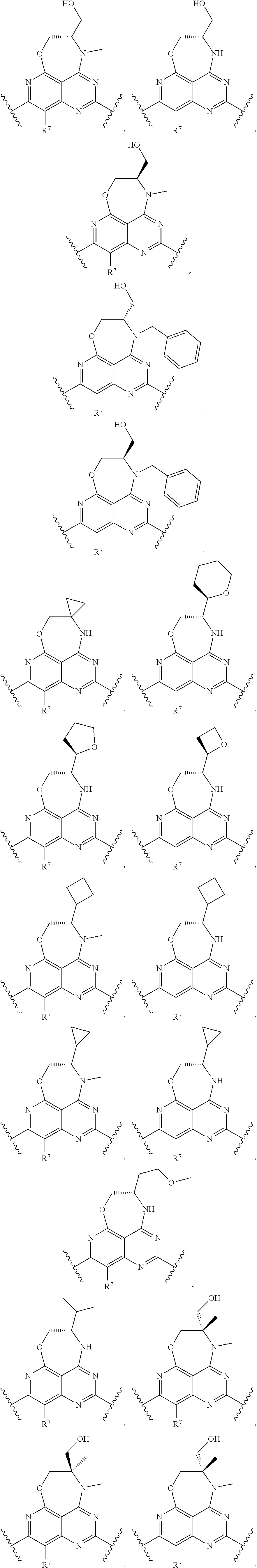 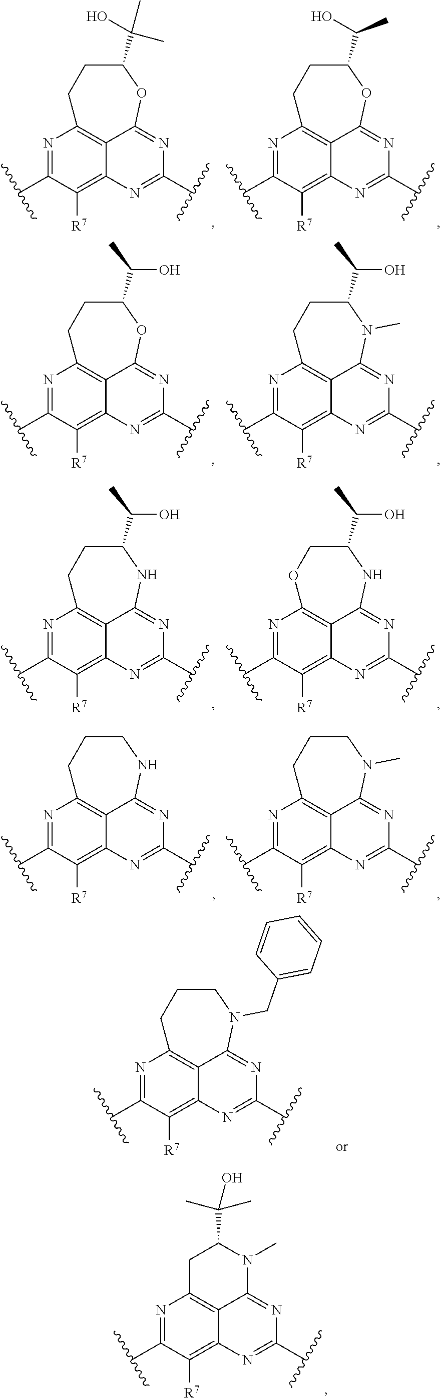
wherein each wherein “ [symbol] ” is a point of covalent attachment to either ring B or —(X)ₚ—R³.

16. The compound of any one of the preceding claims, or a pharmaceutically acceptable salt thereof, wherein Y² is —O—.

17. The compound of any one of the preceding claims, or a pharmaceutically acceptable salt thereof, wherein Y² is —CH₂—.

18. The compound of any one of the preceding claims, or a pharmaceutically acceptable salt thereof, wherein R³ is —C₁-C₆ alkyl, 4- to 10-membered heterocycloalkyl, or —C₁-C₆ alkylene-(4- to 10-membered heterocycloalkyl), wherein each hydrogen atom in —C₁-C₆ alkyl, 4- to 10-membered heterocycloalkyl, or —C₁-C₆ alkylene-(4- to 10-membered heterocycloalkyl), is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

19. The compound of any one of the preceding claims, or a pharmaceutically acceptable salt thereof, wherein R³ is —C₁-C₆ alkylene-(4- to 10-membered heterocycloalkyl), wherein each hydrogen atom in —C₁-C₆ alkylene-(4- to 10-membered heterocycloalkyl), is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

20. The compound of any one of the preceding claims, or a pharmaceutically acceptable salt thereof, wherein R³ is 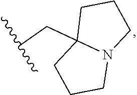 [SMILES: *CC12CCCN1CCC2]
wherein each hydrogen atom is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, and “-” is a point of covalent attachment.

21. The compound of any one of the preceding claims, or a pharmaceutically acceptable salt thereof, wherein R³ is 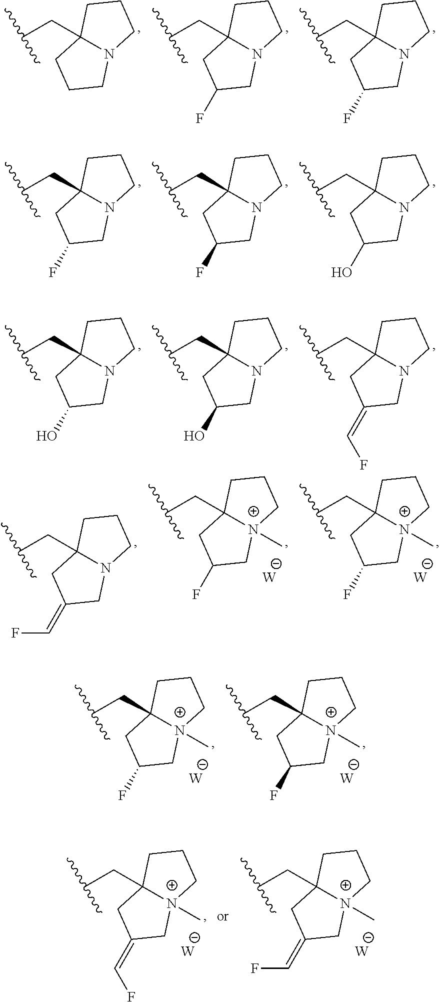
wherein W^{⊖} is an inorganic counter ion or an organic counter ion.

22. The compound of any one of claims 1 to 18, or a pharmaceutically acceptable salt thereof, wherein R³ is 4- to 10-membered heterocycloalkyl, wherein each hydrogen atom in 4- to 10-membered heterocycloalkyl is independently optionally substituted by deuterium, halogen, C₁-C₆ alkyl, —C₁-C₆ alkylene-O—C₁-C₆ alkyl, —OC₁-C₆ alkylene-O—C₁-C₆ alkyl, —C₁-C₆ alkylene-O—R^{a}, C₆-C₁₀ aryl, —C₁-C₆ alkylene-(C₆-C₁₀ aryl), haloalkyl, C₃-C₆ cycloalkyl, 5- to 10-membered heteroaryl, —C₁-C₆ alkylene-(5- to 10-membered heterocycloalkyl), —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}C(═NR^{f})NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

23. The compound of any one of claims 1 to 18, or 22, or a pharmaceutically acceptable salt thereof, wherein R³ is 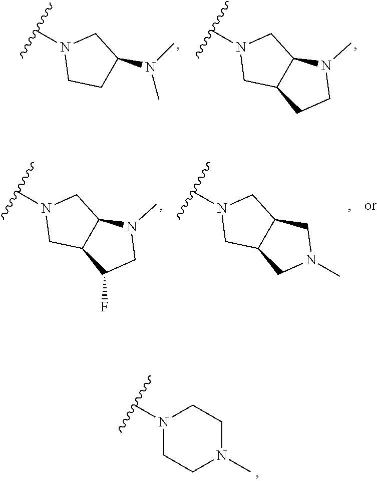
wherein “ [symbol] ” is a point of covalent attachment.

24. The compound of any one of the preceding claims, or a pharmaceutically acceptable salt thereof, wherein m is 1, 2, 3, or 4.

25. The compound of any one of the preceding claims, or a pharmaceutically acceptable salt thereof, wherein n is 0, 1, 2, or 3.

26. The compound of any one of the preceding claims, or a pharmaceutically acceptable salt thereof, wherein Z¹ is N.

27. The compound of any one of the preceding claims, or a pharmaceutically acceptable salt thereof, wherein Z² is N.

28. The compound of any one of the preceding claims, or a pharmaceutically acceptable salt thereof, wherein Z³ is CR⁷ and Z⁴ is N.

29. The compound of any one of claims 1 to 27, or a pharmaceutically acceptable salt thereof, wherein Z³ is CR⁷ and Z⁴ is CR⁸.

30. The compound of any one of claims 1 to 27, or a pharmaceutically acceptable salt thereof, wherein Z³ is N and Z⁴ is CR⁸.

31. The compound of any one of claims 1 to 27, or a pharmaceutically acceptable salt thereof, wherein Z³ is N and Z⁴ is N.

32. The compound of any one of claims 1 to 27, or a pharmaceutically acceptable salt thereof, wherein Z³ is N, Z⁴ is CR⁸, Z⁵ is CR¹⁵, and Z⁶ is CR¹⁶.

33. The compound of any one of claims 1 to 27, or a pharmaceutically acceptable salt thereof, wherein Z³ is CR⁷, Z⁴ is N, Z⁵ is CR¹⁵, and Z⁶ is CR¹⁶.

34. The compound of any one of claims 1 to 27, or a pharmaceutically acceptable salt thereof, wherein Z³ is N, Z⁴ is N, Z⁵ is CR¹⁵, and Z⁶ is CR¹⁶.

35. The compound of any one of the preceding claims, or a pharmaceutically acceptable salt thereof, wherein p is 0.

36. The compound of any one of claims 1 to 34, or a pharmaceutically acceptable salt thereof, wherein p is 1.

37. The compound of any one of claims 1 to 34, or 36, or a pharmaceutically acceptable salt thereof, wherein X is —O—.

38. The compound of any one of claims 1 to 34, or 36, or a pharmaceutically acceptable salt thereof, wherein X is —NR⁴—.

39. The compound of any one of claims 1 to 34, or 36, or a pharmaceutically acceptable salt thereof, wherein X is —S—.

40. The compound of any one of the preceding claims, or a pharmaceutically acceptable salt thereof, wherein each R² is independently deuterium, halogen, C₁-C₆ alkyl, C₂-C₆ alkenyl, C₂-C₆ alkynyl, —OR^{c}, —C(O)R^{c}, —NR^{c}R^{d}, or —CN, wherein each hydrogen atom in C₁-C₆ alkyl, C₂-C₆ alkenyl, and C₂-C₆ alkynyl is independently optionally substituted with a deuterium or halogen.

41. The compound of any one of the preceding claims, or a pharmaceutically acceptable salt thereof, wherein Ring B is 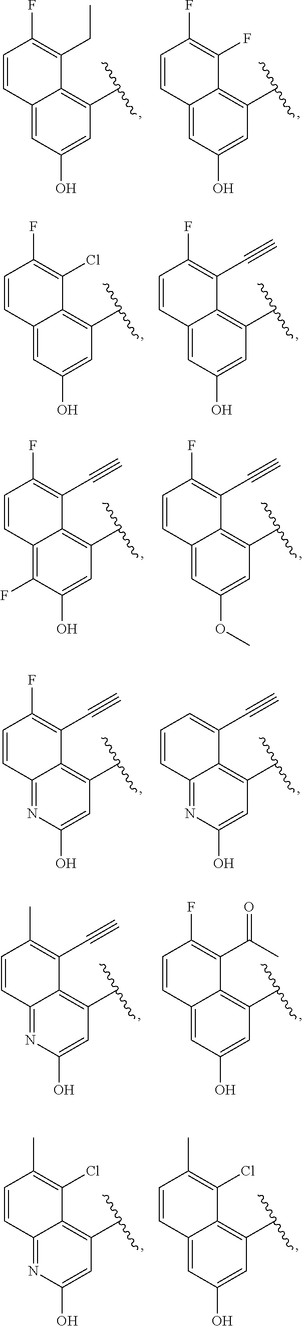 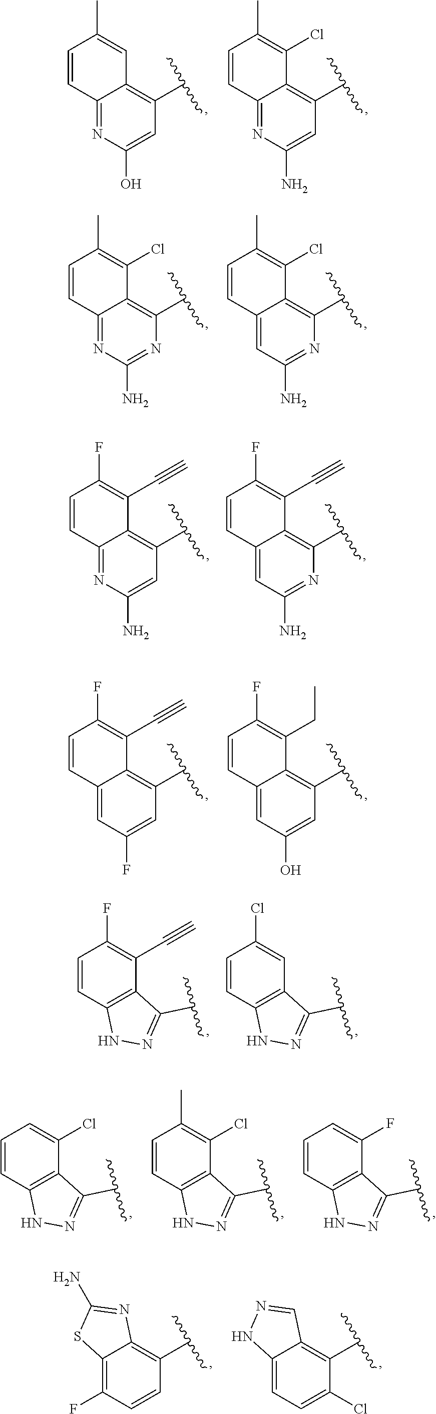 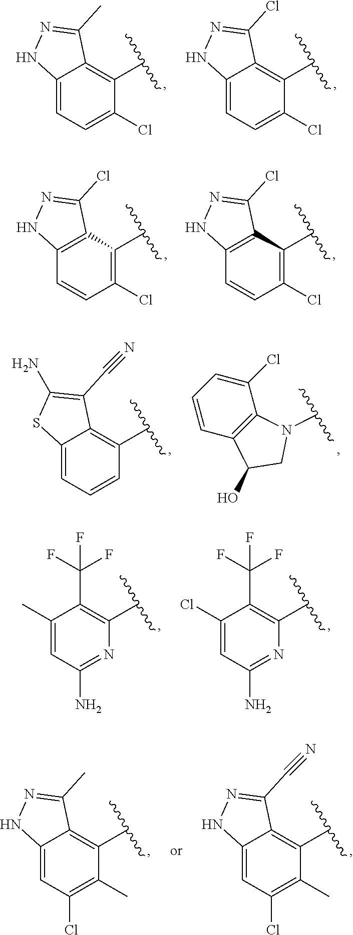
wherein “ [symbol] ” is a point of covalent attachment.

42. The compound of any one of the preceding claims, or a pharmaceutically acceptable salt thereof, wherein R⁴, when present, is H or methyl.

43. The compound of any one of the preceding claims, or a pharmaceutically acceptable salt thereof, wherein R⁵, when present, is H.

44. The compound of any one of the preceding claims, or a pharmaceutically acceptable salt thereof, wherein R⁶, when present, is H.

45. The compound of any one of the preceding claims, or a pharmaceutically acceptable salt thereof, wherein R⁷, when present, is H or F.

46. The compound of any one of the preceding claims, or a pharmaceutically acceptable salt thereof, wherein R⁸, when present, is H.

47. The compound of any one of the preceding claims, or a pharmaceutically acceptable salt thereof, wherein R⁹, when present, is H or methyl.

48. The compound of any one of the preceding claims, or a pharmaceutically acceptable salt thereof, wherein R¹⁰, when present, is H or methyl.

49. The compound of any one of the preceding claims, or a pharmaceutically acceptable salt thereof, wherein R¹¹, when present, is H.

50. The compound of any one of the preceding claims, or a pharmaceutically acceptable salt thereof, wherein R¹², when present, is H.

51. The compound of any one of the preceding claims, or a pharmaceutically acceptable salt thereof, wherein R¹³, when present, is H.

52. The compound of any one of the preceding claims, or a pharmaceutically acceptable salt thereof, wherein R¹⁴, when present, is H.

53. The compound of any one of the preceding claims, or a pharmaceutically acceptable salt thereof, wherein Z⁵, when present, is N.

54. The compound of any one of the preceding claims, or a pharmaceutically acceptable salt thereof, wherein Z⁶, when present, is N.

55. The compound of any one of claims 1 to 51, or a pharmaceutically acceptable salt thereof, wherein Z⁵, when present, is CR¹⁵.

56. The compound of any one of the preceding claims, or a pharmaceutically acceptable salt thereof, wherein R¹⁵, when present, is H.

57. The compound of any one of claims 1 to 53, or a pharmaceutically acceptable salt thereof, wherein Z⁶, when present, is CR¹⁶.

58. The compound of any one of the preceding claims, or a pharmaceutically acceptable salt thereof, wherein R¹⁶, when present, is H.

59. The compound of claim 1, selected from the group consisting of 5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
5-ethyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
(8aR,9R)-5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7-oxa-1,3,6,12a-tetraazabenzo[4,5]cyclohepta[1,2,3-de]naphthalen-9-ol;
(8aR,9R)-5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7H-1,3,6,12a-tetraazabenzo[4,5]cyclohepta[1,2,3-de]naphthalen-9-ol;
(8aR,9R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol;
(8aR,9R)-5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol;
(8aS,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-11-ol;
(8aS,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-11-methyl-8,8a,9,10,11,12-hexahydro-7-oxa-1,3,6,12a-tetraazabenzo[4,5]cyclohepta[1,2,3-de]naphthalen-11-ol;
5-ethyl-6-fluoro-4-[(8S,8aS)-4-fluoro-2-{[(2R,7aS)-2-fluorotetrahydro-1H-pyrrolizin-7a(5H)-yl]methoxy}-8-methyl-8,8a,9,10,11,12-hexahydro-7-oxa-1,3,6,12a-tetraazabenzo[4,5]cyclohepta[1,2,3-de]naphthalen-5-yl]naphthalen-2-ol;
7-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-1,3-benzothiazol-2-amine;
5-ethyl-6-fluoro-4-[(8aS)-4-fluoro-2-{[(2Z,7aS)-2-(fluoromethylidene)tetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
(3S)-7-chloro-1-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-2,3-dihydro-1H-indol-3-ol;
6-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-4-methyl-5-(trifluoromethyl)pyridin-2-amine;
3-chloro-5-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-4-(trifluoromethyl)aniline;
5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydropyrido[2′,1′:3,4][1,4]oxazepino[5,6,7-de]quinazolin-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-(propan-2-yl)-9,10-dihydro-8H-7-oxa-1,3,6,10-tetraazacyclohepta[1,2,3-de]naphthalen-5-yl]naphthalen-2-ol;
5-ethynyl-6-fluoro-4-[(8aS)-4-fluoro-2-{[(2Z,7aS)-2-(fluoromethylidene)tetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
(8aR,9R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7-oxa-1,3,6,12a-tetraazabenzo[4,5]cyclohepta[1,2,3-de]naphthalen-9-ol;
5-ethynyl-6-fluoro-4-[(7aR,10aS)-4-fluoro-2-{1[(2R,7aS)-2-fluorotetrahydro-1H-pyrrolizin-7a(5H)-yl]methoxy}-11-methyl-7a,8,10a,11-tetrahydro-10H-7,9-dioxa-1,3,6,11-tetraazanaphtho[1,8-fg]azulen-5-yl]naphthalen-2-ol;
5-ethynyl-6-fluoro-4-[(7aS,9aS)-4-fluoro-2-{1[(2R,7aS)-2-fluorotetrahydro-1H-pyrrolizin-7a(5H)-yl]methoxy}-10-methyl-8,9,9a,10-tetrahydro-7aH-7-oxa-1,3,6,10-tetraazacyclobuta[5,6]cyclohepta[1,2,3-de]naphthalen-5-yl]naphthalen-2-ol;
5-ethynyl-6-fluoro-4-[(8aS)-4-fluoro-2-{[(2R,7aS)-2-fluorotetrahydro-1H-pyrrolizin-7a(5H)-yl]methoxy}-8a,9,11,12-tetrahydro-8H-7,10-dioxa-1,3,6,12a-tetraazabenzo[4,5]cyclohepta[1,2,3-de]naphthalen-5-yl]naphthalen-2-ol;
5-ethynyl-6-fluoro-4-[(8aS,12S)-4-fluoro-2-{[(2R,7aS)-2-fluorotetrahydro-1H-pyrrolizin-7a(5H)-yl]methoxy}-12-methyl-8a,9,11,12-tetrahydro-8H-7,10-dioxa-1,3,6,12a-tetraazabenzo[4,5]cyclohepta[1,2,3-de]naphthalen-5-yl]naphthalen-2-ol;
(8aS,12S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8a,9,11,12-tetrahydro-8H-7,10-dioxa-1,3,6,12a-tetraazabenzo[4,5]cyclohepta[1,2,3-de]naphthalene-12-carbonitrile;
(8aS,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8a,9,11,12-tetrahydro-8H-7,10-dioxa-1,3,6,12a-tetraazabenzo[4,5]cyclohepta[1,2,3-de]naphthalene-11-carbonitrile;
(8aS,12R)-5-(8-ethynyl-7-fluoro-3-hydroxynaphthalen-1-yl)-4-fluoro-2-{1[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-12-carbonitrile;
(8aS,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-11-carbonitrile;
5-ethynyl-6-fluoro-4-[(8aS)-4-fluoro-2-{[(2R,7aS)-2-fluorotetrahydro-1H-pyrrolizin-7a(5H)-yl]methoxy}-8a,9,12,13-tetrahydro-8H,11H-7,10-dioxa-1,3,6,13a-tetraazanaphtho[1,8-ab]heptalen-5-yl]naphthalen-2-ol;
5-ethynyl-6-fluoro-4-[(8aS,13S)-4-fluoro-2-{[(2R,7aS)-2-fluorotetrahydro-1H-pyrrolizin-7a(5H)-yl]methoxy}-13-methyl-8a,9,12,13-tetrahydro-8H,11H-7,10-dioxa-1,3,6,13a-tetraazanaphtho[1,8-ab]heptalen-5-yl]naphthalen-2-ol;
(8aS,13R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8a,9,12,13-tetrahydro-8H,11H-7,10-dioxa-1,3,6,13a-tetraazanaphtho[1,8-ab]heptalene-13-carbonitrile;
5-ethynyl-6-fluoro-4-[(8aS,12S)-4-fluoro-2-{[(2R,7aS)-2-fluorotetrahydro-1H-pyrrolizin-7a(5H)-yl]methoxy}-12-methyl-8a,9,12,13-tetrahydro-8H,11H-7,10-dioxa-1,3,6,13a-tetraazanaphtho[1,8-ab]heptalen-5-yl]naphthalen-2-ol;
(8aS,12R)-5-(8-ethynyl-7-fluoro-3-hydroxynaphthalen-1-yl)-4-fluoro-2-{1[(2R,7aS)-2-fluorotetrahydro-1H-pyrrolizin-7a(5H)-yl]methoxy}-8a,9,12,13-tetrahydro-8H,11H-7,10-dioxa-1,3,6,13a-tetraazanaphtho[1,8-ab]heptalene-12-carbonitrile;
5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10,12,13-hexahydro-7,11-dioxa-1,3,6,13a-tetraazanaphtho[1,8-ab]heptalen-5-yl]naphthalen-2-ol;
5-ethynyl-6-fluoro-4-{[(8aS,9R)-4-fluoro-2-{1[(2R,7aS)-2-fluorotetrahydro-1H-pyrrolizin-7a(5H)-yl]methoxy}-9-methyl-8,8a,9,10,12,13-hexahydro-7,11-dioxa-1,3,6,13a-tetraazanaphtho[1,8-ab]heptalen-5-yl]naphthalen-2-ol;
5-ethynyl-6-fluoro-4-{[(8aS,13S)-4-fluoro-2-{[(2R,7aS)-2-fluorotetrahydro-1H-pyrrolizin-7a(5H)-yl]methoxy}-13-methyl-8,8a,9,10,12,13-hexahydro-7,11-dioxa-1,3,6,13a-tetraazanaphtho[1,8-ab]heptalen-5-yl]naphthalen-2-ol;
(8aS,13S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,12,13-hexahydro-7,11-dioxa-1,3,6,13a-tetraazanaphtho[1,8-ab]heptalene-13-carbonitrile;
(8aS,12R)-5-(8-ethynyl-7-fluoro-3-hydroxynaphthalen-1-yl)-4-fluoro-2-{1[(2R,7aS)-2-fluorotetrahydro-1H-pyrrolizin-7a(5H)-yl]methoxy}-8,8a,9,10,12,13-hexahydro-7,11-dioxa-1,3,6,13a-tetraazanaphtho[1,8-ab]heptalene-12-carbonitrile;
(8aS)-5-(5-chloro-1H-indazol-4-yl)-4-fluoro-2-{1[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene;
5-ethynyl-6-fluoro-4-[(8aS)-4-fluoro-2-{[(2E)-2-(fluoromethylidene)tetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
(5P,8aS)-5-(3,5-dichloro-1H-indazol-4-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene;
(5M,8aS)-5-(3,5-dichloro-1H-indazol-4-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene;
2-amino-7-fluoro-4-[(8aS)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-1-benzothiophene-3-carbonitrile;
(8aS,11S)-5-(8-ethynyl-7-fluoro-3-hydroxynaphthalen-1-yl)-4-fluoro-2-{1[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-11-ol;
(8aR,11R)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-11-ol;
6-((S)-1-fluoro-11-(((2R,7aS)-2-fluorotetrahydro-1H-pyrrolizin-7a(5H)-yl)methoxy)-5,5a,6,7,8,9-hexahydro-4-oxa-3,9a,10,12-tetraazabenzo[4,5]cyclohepta[1,2,3-de]naphthalen-2-yl)-4-methyl-5-(trifluoromethyl)pyridin-2-amine;
(8aS)-5-(8-ethynyl-3,7-difluoronaphthalen-1-yl)-4-fluoro-2-{1[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene;
(8aR,11S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-11-ol;
(2R,7aS)-7a-({[(8aS)-5-(8-ethynyl-7-fluoro-3-hydroxynaphthalen-1-yl)-4-fluoro-8,8a,9,10,11,12-hexahydro-7-oxa-1,3,6,12a-tetraazabenzo[4,5]cyclohepta[1,2,3-de]naphthalen-2-yl]oxy}methyl)hexahydro-1H-pyrrolizin-2-ol;
5-ethynyl-6-fluoro-4-[(8aS)-4,11,11-trifluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]naphthalen-2-ol;
(2S,7aS)-7a-({[(8aS)-5-(8-ethynyl-7-fluoro-3-hydroxynaphthalen-1-yl)-4-fluoro-8,8a,9,10,11,12-hexahydro-7-oxa-1,3,6,12a-tetraazabenzo[4,5]cyclohepta[1,2,3-de]naphthalen-2-yl]oxy}methyl)hexahydro-1H-pyrrolizin-2-ol;
5-ethynyl-6-fluoro-4-[(7aR,10aS)-4-fluoro-2-{[(2R,7aS)-2-fluorotetrahydro-1H-pyrrolizin-7a(5H)-yl]methoxy}-7a,8,9,10,10a,11-hexahydro-7-oxa-1,3,6,11-tetraazanaphtho[1,8-fg]azulen-5-yl]naphthalen-2-ol;
(8aS,9S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol;
5-ethynyl-6-fluoro-4-[4-fluoro-2-{[(2R,7aS)-2-fluorotetrahydro-1H-pyrrolizin-7a(5H)-yl]methoxy}-13-(hydroxymethyl)-8,9,10,11-tetrahydro-8,12-methano-7-oxa-1,3,6,12-tetraazacyclonona[1,2,3-de]naphthalen-5-yl]naphthalen-2-ol;
4-[(8aS)-4,11-difluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-5-yl]-5-ethynyl-6-fluoronaphthalen-2-ol;
5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,12-tetrahydro-7-oxa-1,3,6,12a-tetraazabenzo[4,5]cyclohepta[1,2,3-de]naphthalen-5-yl]naphthalen-2-ol;
5-ethynyl-6-fluoro-4-[(8aS)-4-fluoro-2-{[(2R,7aS)-2-fluorotetrahydro-1H-pyrrolizin-7a(5H)-yl]methoxy}-8,8a,9,10-tetrahydro-7-oxa-1,3,6,12a-tetraazabenzo[4,5]cyclohepta[1,2,3-de]naphthalen-5-yl]naphthalen-2-ol;
5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-11-carbonitrile;
5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-11-carboxamide;
(8aS,9S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7-oxa-1,3,6,12a-tetraazabenzo[4,5]cyclohepta[1,2,3-de]naphthalen-9-ol;
(8aS,9S)-5-(8-ethyl-7-fluoro-3-hydroxynaphthalen-1-yl)-4-fluoro-2-{[(2R,7aS)-2-fluorotetrahydro-1H-pyrrolizin-7a(5H)-yl]methoxy}-9-methyl-8,8a,9,10,11,12-hexahydro-7-oxa-1,3,6,12a-tetraazabenzo[4,5]cyclohepta[1,2,3-de]naphthalen-9-ol;
(8aS)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalene-11-carbonitrile;
5-(8-ethyl-7-fluoro-3-hydroxynaphthalen-1-yl)-4-fluoro-2-{1[(2R,7aS)-2-fluorotetrahydro-1H-pyrrolizin-7a(5H)-yl]methoxy}-8,8a,9,10,11,12-hexahydro-7-oxa-1,3,6,12a-tetraazabenzo[4,5]cyclohepta[1,2,3-de]naphthalen-9-ol;
(8aS,11S)-5-(8-ethynyl-7-fluoro-3-hydroxynaphthalen-1-yl)-4-fluoro-2-{[(2R,7aS)-2-fluorotetrahydro-1H-pyrrolizin-7a(5H)-yl]methoxy}-11-methyl-8,8a,9,10,11,12-hexahydro-7-oxa-1,3,6,12a-tetraazabenzo[4,5]cyclohepta[1,2,3-de]naphthalen-11-ol; and
5-ethynyl-6-fluoro-4-(4-fluoro-2-{1[(2R,7aS)-2-fluorotetrahydro-1H-pyrrolizin-7a(5H)-yl]methoxy}-8,8a,9,10,11,12-hexahydro-7H-1,3,6,12a-tetraazabenzo[4,5]cyclohepta[1,2,3-de]naphthalen-5-yl)naphthalen-2-ol;
or a pharmaceutically acceptable salt thereof.

60. The compound of claim 1, selected from the group consisting of:
5-ethynyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-9,10-dimethyl-9,10-dihydro-8H-7-oxa-1,3,6,10-tetraazacyclohepta[1,2,3-de]naphthalen-5-yl]naphthalen-2-ol;
5-ethynyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-9,10-dimethyl-9,10-dihydro-8H-7-oxa-1,3,6,10-tetraazacyclohepta[1,2,3-de]naphthalen-5-yl]naphthalen-2-ol;
5-ethyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-9,10-dimethyl-9,10-dihydro-8H-7-oxa-1,3,6,10-tetraazacyclohepta[1,2,3-de]naphthalen-5-yl]naphthalen-2-ol;
5-ethynyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-9,10-dimethyl-7,8,9,10-tetrahydro-1,3,6,10-tetraazacyclohepta[1,2,3-de]naphthalen-5-yl]naphthalen-2-ol;
5-ethyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-9,10-dimethyl-7,8,9,10-tetrahydro-1,3,6,10-tetraazacyclohepta[1,2,3-de]naphthalen-5-yl]naphthalen-2-ol;
5-ethynyl-6-fluoro-4-[(9R)-4-fluoro-2-{[(2R,7aS)-2-fluorotetrahydro-1H-pyrrolizin-7a(5H)-yl]methoxy}-9-(2-hydroxypropan-2-yl)-8,9-dihydro-7H-10-oxa-1,3,6-triazacyclohepta[1,2,3-de]naphthalen-5-yl]naphthalen-2-ol;
5-ethyl-6-fluoro-4-[(9R)-4-fluoro-2-{[(2R,7aS)-2-fluorotetrahydro-1H-pyrrolizin-7a(5H)-yl]methoxy}-9-(2-hydroxypropan-2-yl)-8,9-dihydro-7H-10-oxa-1,3,6-triazacyclohepta[1,2,3-de]naphthalen-5-yl]naphthalen-2-ol;
5-ethynyl-6-fluoro-4-{(9R)-4-fluoro-2-{1[(2R,7aS)-2-fluorotetrahydro-1H-pyrrolizin-7a(5H)-yl]methoxy}-9-[(1S)-1-hydroxyethyl]-8,9-dihydro-7H-10-oxa-1,3,6-triazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1S)-1-hydroxyethyl]-8,9-dihydro-7H-10-oxa-1,3,6-triazacyclohepta[1,2,3-de]naphthalen-5-yl}naphthalen-2-ol;
5-ethynyl-6-fluoro-4-{(9R)-4-fluoro-2-{1[(2R,7aS)-2-fluorotetrahydro-1H-pyrrolizin-7a(5H)-yl]methoxy}-9-[(1R)-1-hydroxyethyl]-8,9-dihydro-7H-10-oxa-1,3,6-triazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-8,9-dihydro-7H-10-oxa-1,3,6-triazacyclohepta[1,2,3-de]naphthalen-5-yl}naphthalen-2-ol;
5-ethynyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-10-methyl-7,8,9,10-tetrahydro-1,3,6,10-tetraazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-10-methyl-7,8,9,10-tetrahydro-1,3,6,10-tetraazacyclohepta[1,2,3-de]naphthalen-5-yl}naphthalen-2-ol;
5-ethynyl-6-fluoro-4-{(9R)-4-fluoro-2-{1[(2R,7aS)-2-fluorotetrahydro-1H-pyrrolizin-7a(5H)-yl]methoxy}-9-[(1R)-1-hydroxyethyl]-7,8,9,10-tetrahydro-1,3,6,10-tetraazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-7,8,9,10-tetrahydro-1,3,6,10-tetraazacyclohepta[1,2,3-de]naphthalen-5-yl}naphthalen-2-ol;
5-ethynyl-6-fluoro-4-{(9R)-4-fluoro-2-{1[(2R,7aS)-2-fluorotetrahydro-1H-pyrrolizin-7a(5H)-yl]methoxy}-9-[(1R)-1-hydroxyethyl]-9,10-dihydro-8H-7-oxa-1,3,6,10-tetraazacyclohepta[1,2,3-de]naphthalen-5-yl}naphthalen-2-ol;
5-ethyl-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-9,10-dihydro-8H-7-oxa-1,3,6,10-tetraazacyclohepta[1,2,3-de]naphthalen-5-yl}naphthalen-2-ol;
5-ethynyl-6-fluoro-4-[(5R)-9-fluoro-2-{[(2R,7aS)-2-fluorotetrahydro-1H-pyrrolizin-7a(5H)-yl]methoxy}-5-(2-hydroxypropan-2-yl)-4-methyl-5,6-dihydro-4H-pyrimido[4,5,6-de][1,6]naphthyridin-8-yl]naphthalen-2-ol;
5-ethyl-6-fluoro-4-[(5R)-9-fluoro-2-{[(2R,7aS)-2-fluorotetrahydro-1H-pyrrolizin-7a(5H)-yl]methoxy}-5-(2-hydroxypropan-2-yl)-4-methyl-5,6-dihydro-4H-pyrimido[4,5,6-de][1,6]naphthyridin-8-yl]naphthalen-2-ol;
5-chloro-6-fluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-7,8,9,10-tetrahydro-1,3,6,10-tetraazacyclohepta[1,2,3-de]naphthalen-5-yl}naphthalen-2-ol;
5,6-difluoro-4-{(9R)-4-fluoro-2-{[(2R,7aS)-2-fluorotetrahydro-1H-pyrrolizin-7a(5H)-yl]methoxy}-9-[(1R)-1-hydroxyethyl]-7,8,9,10-tetrahydro-1,3,6,10-tetraazacyclohepta[1,2,3-de]naphthalen-5-yl}naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-10-methyl-9,10-dihydro-8H-7-oxa-1,3,6,10-tetraazacyclohepta[1,2,3-de]naphthalen-5-yl]naphthalen-2-ol;
5-ethyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-10-methyl-9,10-dihydro-8H-7-oxa-1,3,6,10-tetraazacyclohepta[1,2,3-de]naphthalen-5-yl]naphthalen-2-ol;
4-[(9S)-9-cyclopropyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethynyl-6-fluoronaphthalen-2-ol;
4-[(9S)-9-cyclopropyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-5-ethynyl-6-fluoronaphthalen-2-ol;
4-[(9S)-9-cyclopropyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethyl-6-fluoronaphthalen-2-ol;
4-[(9S)-9-cyclopropyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-5-ethyl-6-fluoronaphthalen-2-ol;
4-[(9S)-9-cyclobutyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethynyl-6-fluoronaphthalen-2-ol;
5-ethynyl-6-fluoro-4-{(9R)-4-fluoro-2-{1[(2R,7aS)-2-fluorotetrahydro-1H-pyrrolizin-7a(5H)-yl]methoxy}-9-[(2R)-oxolan-2-yl]-9,10-dihydro-8H-7-oxa-1,3,6,10-tetraazacyclohepta[1,2,3-de]naphthalen-5-yl}naphthalen-2-ol;
5-ethynyl-6-fluoro-4-{(9R)-4-fluoro-2-{1[(2R,7aS)-2-fluorotetrahydro-1H-pyrrolizin-7a(5H)-yl]methoxy}-9-[(2R)-oxan-2-yl]-9,10-dihydro-8H-7-oxa-1,3,6,10-tetraazacyclohepta[1,2,3-de]naphthalen-5-yl}naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-methyl-9,10-dihydro-8H-7-oxa-1,3,6,10-tetraazacyclohepta[1,2,3-de]naphthalen-5-yl]naphthalen-2-ol;
5-ethynyl-6-fluoro-4-{(9R)-4-fluoro-2-{1[(2R,7aS)-2-fluorotetrahydro-1H-pyrrolizin-7a(5H)-yl]methoxy}-9-[(2R)-oxetan-2-yl]-9,10-dihydro-8H-7-oxa-1,3,6,10-tetraazacyclohepta[1,2,3-de]naphthalen-5-yl}naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-methyl-10-(propan-2-yl)-9,10-dihydro-8H-7-oxa-1,3,6,10-tetraazacyclohepta[1,2,3-de]naphthalen-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-methyl-10-(oxetan-3-yl)-9,10-dihydro-8H-7-oxa-1,3,6,10-tetraazacyclohepta[1,2,3-de]naphthalen-5-yl]naphthalen-2-ol;
5-ethynyl-6-fluoro-4-[(9S)-4-fluoro-2-{[(2R)-2-fluorotetrahydro-1H-pyrrolizin-7a(5H)-yl]methoxy}-9-(propan-2-yl)-9,10-dihydro-8H-7-oxa-1,3,6,10-tetraazacyclohepta[1,2,3-de]naphthalen-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9-(2-methoxyethyl)-9,10-dihydro-8H-7-oxa-1,3,6,10-tetraazacyclohepta[1,2,3-de]naphthalen-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-9,10-dimethyl-9,10-dihydro-8H-7-oxa-1,3,6,10-tetraazacyclohepta[1,2,3-de]naphthalen-5-yl]naphthalen-2-ol;
5-ethynyl-6-fluoro-4-[(9S)-4-fluoro-2-{[(2R,7aS)-2-fluorotetrahydro-1H-pyrrolizin-7a(5H)-yl]methoxy}-10-methyl-9-(propan-2-yl)-9,10-dihydro-8H-7-oxa-1,3,6,10-tetraazacyclohepta[1,2,3-de]naphthalen-5-yl]naphthalen-2-ol;
4-[(9S)-10-benzyl-4-fluoro-2-{[(2R,7aS)-2-fluorotetrahydro-1H-pyrrolizin-7a(5H)-yl]methoxy}-9-methyl-9,10-dihydro-8H-7-oxa-1,3,6,10-tetraazacyclohepta[1,2,3-de]naphthalen-5-yl]-5-ethynyl-6-fluoronaphthalen-2-ol;
(2R,7aS)-7a-({[(9S)-5-(8-ethynyl-7-fluoro-3-hydroxynaphthalen-1-yl)-4-fluoro-9-methyl-9,10-dihydro-8H-7-oxa-1,3,6,10-tetraazacyclohepta[1,2,3-de]naphthalen-2-yl]oxy}methyl)-2-fluoro-4-methylhexahydro-1H-pyrrolizin-4-ium;
4-[(9S)-10-benzyl-4-fluoro-2-{[(2R,7aS)-2-fluorotetrahydro-1H-pyrrolizin-7a(5H)-yl]methoxy}-9-(hydroxymethyl)-9,10-dihydro-8H-7-oxa-1,3,6,10-tetraazacyclohepta[1,2,3-de]naphthalen-5-yl]-5-ethynyl-6-fluoronaphthalen-2-ol;
4-[(9R)-10-benzyl-4-fluoro-2-{[(2R,7aS)-2-fluorotetrahydro-1H-pyrrolizin-7a(5H)-yl]methoxy}-9-(hydroxymethyl)-9,10-dihydro-8H-7-oxa-1,3,6,10-tetraazacyclohepta[1,2,3-de]naphthalen-5-yl]-5-ethynyl-6-fluoronaphthalen-2-ol;
5-ethynyl-6-fluoro-4-(4-fluoro-2-{1[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl)naphthalen-2-ol;
4-[(9S)-9-ethyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl]-5-ethynyl-6-fluoronaphthalen-2-ol;
5-ethynyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-10-methyl-9,10-dihydro-8H-7-oxa-1,3,6,10-tetraazacyclohepta[1,2,3-de]naphthalen-5-yl]naphthalen-2-ol;
5-ethyl-6-fluoro-4-[(9R)-4-fluoro-2-{[(2R,7aS)-2-fluorotetrahydro-1H-pyrrolizin-7a(5H)-yl]methoxy}-9-(hydroxymethyl)-10-methyl-9,10-dihydro-8H-7-oxa-1,3,6,10-tetraazacyclohepta[1,2,3-de]naphthalen-5-yl]naphthalen-2-ol;
5-ethynyl-6-fluoro-4-(4′-fluoro-2′-{[(2R,7aS)-2-fluorotetrahydro-1H-pyrrolizin-7a(5H)-yl]methoxy}-10′-methyl-8′H,10′H-spiro[cyclopropane-1,9′-[7]oxa[1,3,6,10]tetraazacyclohepta[1,2,3-de]naphthalen]-5′-yl)naphthalen-2-ol;
5-ethynyl-6-fluoro-4-(4′-fluoro-2′-{[(2R,7aS)-2-fluorotetrahydro-1H-pyrrolizin-7a(5H)-yl]methoxy}-8′H,10′H-spiro[cyclopropane-1,9′-[7]oxa[1,3,6,10]tetraazacyclohepta[1,2,3-de]naphthalen]-5′-yl)naphthalen-2-ol;
4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-5-ethynyl-6-fluoronaphthalen-2-ol;
5-ethyl-4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-6-fluoronaphthalen-2-ol;
4-(10-ethyl-4-fluoro-2-{[(2R,7aS)-2-fluorotetrahydro-1H-pyrrolizin-7a(5H)-yl]methoxy}-9,10-dihydro-8H-7-oxa-1,3,6,10-tetraazacyclohepta[1,2,3-de]naphthalen-5-yl)-5-ethynyl-6-fluoronaphthalen-2-ol;
5-chloro-4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-6-fluoronaphthalen-2-ol;
4-(10-benzyl-4-fluoro-2-{[(2R,7aS)-2-fluorotetrahydro-1H-pyrrolizin-7a(5H)-yl]methoxy}-7,8,9,10-tetrahydro-1,3,6,10-tetraazacyclohepta[1,2,3-de]naphthalen-5-yl)-5-ethynyl-6-fluoronaphthalen-2-ol;
4-[(9S)-9-ethyl-4-fluoro-2-{[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-5,6-difluoronaphthalen-2-ol;
2-amino-4-[(9S)-9-ethyl-4-fluoro-2-{1[(2R,7aS)-2-fluorotetrahydro-1H-pyrrolizin-7a(5H)-yl]methoxy}-10-methyl-9,10-dihydro-8H-7-oxa-1,3,6,10-tetraazacyclohepta[1,2,3-de]naphthalen-5-yl]-1-benzothiophene-3-carbonitrile;
5-ethyl-6-fluoro-4-(4-fluoro-2-{[(2R,7aS)-2-fluorotetrahydro-1H-pyrrolizin-7a(5H)-yl]methoxy}-7,8,9,10-tetrahydro-1,3,6,10-tetraazacyclohepta[1,2,3-de]naphthalen-5-yl)naphthalen-2-ol;
5-ethynyl-6-fluoro-4-(4-fluoro-2-{1[(2R,7aS)-2-fluorotetrahydro-1H-pyrrolizin-7a(5H)-yl]methoxy}-7,8,9,10-tetrahydro-1,3,6,10-tetraazacyclohepta[1,2,3-de]naphthalen-5-yl)naphthalen-2-ol;
5-ethyl-6-fluoro-4-[(9S)-4-fluoro-2-{[(2R,7aS)-2-fluorotetrahydro-1H-pyrrolizin-7a(5H)-yl]methoxy}-9,10-dimethyl-9,10-dihydro-8H-7-oxa-1,3,6,10-tetraazacyclohepta[1,2,3-de]naphthalen-5-yl]naphthalen-2-ol;
5-ethyl-6-fluoro-4-[(9S)-4-fluoro-2-{[(2R,7aS)-2-fluorotetrahydro-1H-pyrrolizin-7a(5H)-yl]methoxy}-9-(hydroxymethyl)-9,10-dihydro-8H-7-oxa-1,3,6,10-tetraazacyclohepta[1,2,3-de]naphthalen-5-yl]naphthalen-2-ol;
1-[8-(10-cyclopropyl-4-fluoro-2-{[(2R,7aS)-2-fluorotetrahydro-1H-pyrrolizin-7a(5H)-yl]methoxy}-9-methyl-9,10-dihydro-8H-7-oxa-1,3,6,10-tetraazacyclohepta[1,2,3-de]naphthalen-5-yl)-2-fluoro-6-hydroxynaphthalen-1-yl]ethan-1-one;
4-(9-cyclopropyl-4-fluoro-2-(((2R,7aS)-2-fluorotetrahydro-1H-pyrrolizin-7a(5H)-yl)methoxy)-10-methyl-9,10-dihydro-8H-7-oxa-1,3,6,10-tetraazacyclohepta[de]naphthalen-5-yl)-5-ethynyl-6-fluoronaphthalen-2-ol;
4-(9-cyclopropyl-4-fluoro-2-(((2R,7aS)-2-fluorotetrahydro-1H-pyrrolizin-7a(5H)-yl)methoxy)-10-methyl-9,10-dihydro-8H-7-oxa-1,3,6,10-tetraazacyclohepta[de]naphthalen-5-yl)-5-ethynyl-6-fluoronaphthalen-2-ol;
5-ethyl-6-fluoro-4-((5aS)-1-fluoro-11-(((2R,7aS)-2-fluorotetrahydro-1H-pyrrolizin-7a(5H)-yl)methoxy)-5-methyl-5,5a,6,7,8,9-hexahydro-4-oxa-3,9a,10,12-tetraazabenzo[4,5]cyclohepta[1,2,3-de]naphthalen-2-yl)naphthalen-2-ol;
4-((S)-10-ethyl-4-fluoro-2-(((2R,7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-5-ethynyl-6-fluoronaphthalen-2-ol;
4-((S)-10-(2,2-difluoroethyl)-4-fluoro-2-(((2R,7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-5-ethynyl-6-fluoronaphthalen-2-ol; 5-ethynyl-6-fluoro-4-(4-fluoro-2-(((2R,7aS)-2-fluorotetrahydro-1H-pyrrolizin-7a(5H)-yl)methoxy)-10-methyl-4′,5′-dihydro-2′H,8H,10H-7-oxa-1,3,6,10-tetraazaspiro[cyclohepta[de]naphthalene-9,3′-furan]-1 (10a),2,3a,3a1 (6a),5-pentaen-5-yl)naphthalen-2-ol;
5-ethynyl-6-fluoro-4-((S)-4-fluoro-2-(((2R,7aS)-2-fluorotetrahydro-1H-pyrrolizin-7a(5H)-yl)methoxy)-10-((R)-2-hydroxypropyl)-9-methyl-9,10-dihydro-8H-7-oxa-1,3,6,10-tetraazacyclohepta[de]naphthalen-5-yl)naphthalen-2-ol;
4-((S)-10-(2,2-difluoroethyl)-4-fluoro-2-(((2R,7aS)-2-fluorotetrahydro-1H-pyrrolizin-7a(5H)-yl)methoxy)-9-methyl-9,10-dihydro-8H-7-oxa-1,3,6,10-tetraazacyclohepta[de]naphthalen-5-yl)-5-ethyl-6-fluoronaphthalen-2-ol;
5-ethyl-6-fluoro-4-(4-fluoro-2-(((2R,7aS)-2-fluorotetrahydro-1H-pyrrolizin-7a(5H)-yl)methoxy)-10-methyl-4′,5′-dihydro-2′H,8H,10H-7-oxa-1,3,6,10-tetraazaspiro[cyclohepta[de]naphthalene-9,3′-furan]-1 (10a),2,3a,3a1 (6a),5-pentaen-5-yl)naphthalen-2-ol; and
5-ethynyl-6-fluoro-4-((S)-4-fluoro-2-(((2R,7aS)-2-fluorotetrahydro-1H-pyrrolizin-7a(5H)-yl)methoxy)-9-methyl-10-(2,2,2-trifluoroethyl)-9,10-dihydro-8H-7-oxa-1,3,6,10-tetraazacyclohepta[de]naphthalen-5-yl)naphthalen-2-ol;
or a pharmaceutically acceptable salt thereof.

61. A pharmaceutical composition comprising at least one compound of any one of claims 1 to 60, or a pharmaceutically acceptable salt thereof, and optionally one or more pharmaceutically acceptable excipients.

62. A method of treating disease, such as cancer, comprising administering to a subject in need of such treatment an effective amount of a compound of any one of claims 1 to 60, or a pharmaceutically acceptable salt thereof.

63. A compound of any one of claims 1 to 60, or a pharmaceutically acceptable salt thereof, for use in a method of treating cancer in a subject.

64. A compound of any one of claims 1 to 60, or a pharmaceutically acceptable salt thereof, for treating cancer in a subject.

65. Use of a compound of any one of claims 1 to 60, or a pharmaceutically acceptable salt thereof, in the manufacture of a medicament for treating cancer in a subject.
